(19) United States
(12) Patent Application Publication
(10) Pub. No.: US 2026/0068523 A1
(43) Pub. Date: Mar. 5, 2026

(54) ORGANIC COMPOUNDS AND ORGANIC LIGHT EMITTING DIODE COMPRISING THE SAME

(71) Applicant: Material Science Co., Ltd., Hwaseong-si (KR)

(72) Inventors: Tae Wan LEE, Hwaseong-si (KR); Ha Ni JEON, Hwaseong-si (KR); Da Hee PARK, Hwaseong-si (KR); Sang Mee KIM, Hwaseong-si (KR); Seung Uk CHO, Hwaseong-si (KR)

(21) Appl. No.: 19/319,296

(22) Filed: Sep. 4, 2025

(30) Foreign Application Priority Data
Sep. 5, 2024 (KR) 10-2024-0120850
Sep. 1, 2025 (KR) 10-2025-0123060

(51) Int. Cl. H10K 85/60; C07D 307/91; C07D 407/12; C07D 409/12; H10K 50/15

(52) U.S. Cl. CPC H10K 85/636; C07D 307/91; C07D 407/12; C07D 409/12; H10K 85/633; H10K 50/156; H10K 85/615; H10K 85/626; H10K 85/6574

(57) ABSTRACT

The organic compound represented by Chemical Formula 1 according to the present disclosure may exhibit excellent hole transport properties. Further, the hole transport layer or hole transport auxiliary layer of the organic light emitting diode according to the present disclosure includes the organic compound represented by Chemical Formula 1 according to the present disclosure, thereby improving the driving voltage, efficiency, and lifetime characteristics of the organic light emitting diode.

### CROSS-REFERENCE TO RELATED APPLICATION(S)

This application claims benefit of priority to Korean Patent Application No. 10-2024-0120850 filed on Sep. 5, 2024 and No. 10-2025-0123060 filed on Sep. 1, 2025 in the Korean Intellectual Property Office, the disclosure of which is incorporated herein by reference in its entirety.

### TECHNICAL FIELD

The present disclosure relates to an organic compound and an organic light emitting diode comprising the same.

### BACKGROUND

Organic light emitting diodes (OLEDs) have been actively developed and commercialized as light sources for flat panel displays such as wall-mountable televisions, backlights for displays, lighting devices, and signboards, because they have simplified structure, and various advantages in manufacturing processes, high luminance, excellent viewing angle characteristics, a fast response speed, and a low driving voltage, compared to other flat panel display devices such as conventional liquid crystal displays (LCDs), plasma display panels (PDPs), and field emission displays (FEDs).

The OLED comprises two electrodes and an organic layer disposed between the two electrodes. The OLED is an element that uses the principle that electrons and holes are injected into an emitting layer from the two electrodes, respectively, and are combined with each other in the emitting layer to generate excitons and light is generated when the generated excitons drop from an excited state to a ground state.

The OLED may comprise at least one emitting layer. In general, an OLED comprising a plurality of emitting layers may include emitting layer(s) that emit light having different peak wavelengths, and a specific color may be implemented through a combination of light having the different peak wavelengths.

Such an OLED may be categorized into a top-emission structure and a bottom-emission structure. The top-emission OLED emits light generated in an emitting layer toward a translucent anode using a reflective cathode. In contrast, the bottom-emission OLED emits light generated in an emitting layer and reflected by an anode toward a transparent cathode, which is a direction toward a driving thin film transistor, using the reflective anode.

### PATENT DOCUMENT

[Patent Document 1]WO 2020/111253 A1 (Published: Jun. 4, 2020)

### SUMMARY

An object of the present disclosure is to provide a novel organic compound and an organic light emitting diode comprising the same.

Embodiments according to the present disclosure may be used to achieve other problems not specifically mentioned, in addition to the above problems.

The object of the present disclosure is not limited to those described above, and other objects and advantages of the present disclosure not mentioned, can be understood from the following description and will be more clearly understood from the embodiments of the present disclosure.

Furthermore, it will be readily apparent that the objects and advantages of the present disclosure may be realized by means and combinations thereof set forth in the claims.

To solve the above problems, according to an embodiment of the present disclosure, there is provided an organic compound represented by the following Chemical Formula 1:

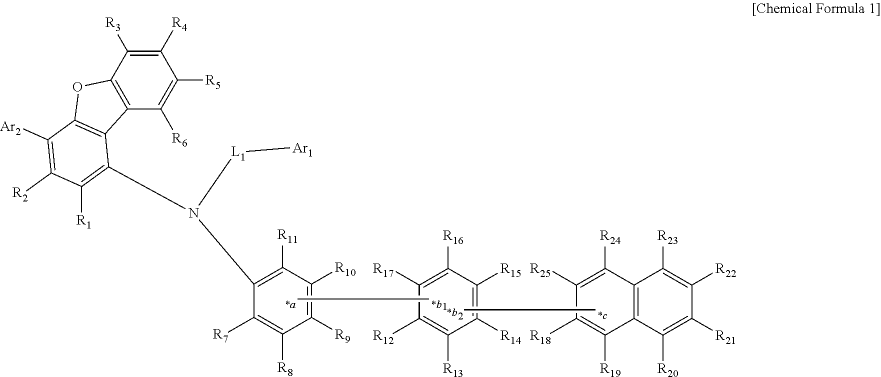

wherein in the chemical formula 1,
L₁ is selected from the group consisting of a single bond, a substituted or unsubstituted arylene group having 6 to 30 carbon atoms, and a substituted or unsubstituted heteroarylene group having 1 to 30 carbon atoms;
Ar₁ and Ar₂ are identical to or different from each other, and are each independently selected from the group consisting of a substituted or unsubstituted aryl group having 6 to 30 carbon atoms, and a substituted or unsubstituted heteroaryl group having 1 to 30 carbon atoms;
R₁ to R₂₅ are identical to or different from each other, and are each independently selected from hydrogen, deuterium, a substituted or unsubstituted aryl group having 6 to 30 carbon atoms, and a substituted or unsubstituted heteroaryl group having 1 to 30 carbon atoms;
When L₁, Ar₁, Ar₂, and R₁ to R₂₅ are substituted, the substituents are identical to or different from each other, and may each independently be one or more selected from the group consisting of deuterium, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, a heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms, wherein when a plurality of the substituents are present, the substituents are identical to or different from each other, and may, together with adjacent groups, form a substituted or unsubstituted ring structure;
*a, *b₁, and *b₂ are different from each other, and represent bonding positions on a phenylene moiety, and *c represents a bonding position on a naphthyl moiety;
one of R₇ to R₁₁ is a single bond that is bonded to *a;
one of R₁₂ to R₁₇ is a single bond that is bonded to *b₁;
one of R₁₂ to R₁₇ is a single bond that is bonded to *b₂; and
one of R₁₈ to R₂₅ is a single bond that is bonded to *c.

According to another embodiment of the present disclosure, there is provided an organic light emitting diode comprising: an anode; a cathode facing the anode; and one or more organic layers disposed between the anode and the cathode, wherein at least one of the organic layers includes the organic compound represented by Chemical Formula 1, and wherein the organic layer including the organic compound represented by Chemical Formula 1 is a hole transport layer or a hole transport auxiliary layer.

The organic compound represented by Chemical Formula 1 according to the present disclosure may exhibit excellent hole transport properties.

In addition, when the hole transport layer and/or a hole transport auxiliary layer incudes the organic compound represented by Chemical Formula 1 according to the present disclosure, the driving voltage, efficiency, and lifetime characteristics of the organic light emitting diode according to the present disclosure may be improved.

Furthermore, when the organic compound represented by Chemical Formula 1 according to the present disclosure is used as a material for the hole transport auxiliary layer, it may have an energy level suitable for serving as a hole transport auxiliary layer that transfers holes from the hole transport layer to the emitting layer and blocks electrons coming from the emitting layer.

Moreover, the organic light emitting diode according to the present disclosure may excellently realize a target color coordinates of the emitting layer, even when a hole transport layer and/or a hole transport auxiliary layer including the organic compound represented by Chemical Formula 1 according to the present disclosure is combined with an emitting layer of any color. The effects of the present disclosure are not limited to those described above, and other effects not mentioned will be clearly understood by those skilled in the art from the following description.

### DETAILED DESCRIPTION

The aforementioned objects, features, and advantages will be described in detail below, and accordingly, those skilled in the art to which the present disclosure pertains will be able to easily implement the technical spirit of the present disclosure.

In the description of the present disclosure, detailed descriptions of known technologies related to the present disclosure will be omitted if they are deemed to unnecessarily obscure the gist of the present disclosure.

As used herein, it is to be understood that when terms such as “comprises,” “has,” “consists of,” “arranges,” “provides,” etc. are used with respect to components, additional components may be present, unless the term “only” is used. Also, it is to be understood that, unless expressly stated otherwise, when a component is referred to in the singular, it is intended to include the plural.

In interpreting the components in the present specification, it is to be understood that the ranges include allowable tolerances even if not explicitly stated.

As used herein, it is to be understood that when any configuration is described as being disposed “on (or under)” a component or “on an upper portion (or lower portion)” of a component, any configuration may be disposed not only in contact with the top (or bottom) surface of the component, but also that other components may intervene between the component and any component disposed on (or under) the component.

As used herein, the term “halogen group” includes fluorine, chlorine, bromine, and iodine.

As used herein, the term “alkyl group” refers to both a straight-chain alkyl radical and a branched-chain alkyl radical. Unless otherwise specified, the alkyl group contains 1 to 10 carbon atoms and may include, but is not limited to, methyl, ethyl, propyl, isopropyl, butyl, sec-butyl, isobutyl, tert-butyl, pentyl, isoamyl, hexyl, etc. Additionally, the alkyl group may be optionally substituted.

As used herein, the term “cycloalkyl group” refers to a cyclic alkyl radical. Unless otherwise specified, the cycloalkyl group contains 3 to 10 carbon atoms and may include, but is not limited to, cyclopropyl, cyclobutyl, cyclopentyl, cyclohexyl, norbornyl, adamantyl, etc. Additionally, the cycloalkyl group may be optionally substituted.

As used herein, the term “alkenyl group” refers to both a straight-chain alkenyl radical and a branched-chain alkenyl radical having one or more carbon-carbon double bonds. Unless otherwise specified, the alkenyl group contains 2 to 30 carbon atoms and may include, but is not limited to, vinyl, allyl, isopropenyl, 2-butenyl, etc. Additionally, the alkenyl group may be optionally substituted.

As used herein, the term “cycloalkenyl group” refers to a cyclic alkenyl radical. Unless otherwise specified, the cycloalkenyl group contains 3 to 20 carbon atoms. Additionally, the cycloalkenyl group may be optionally substituted.

As used herein, the term “alkynyl group” refers to both a straight-chain alkynyl radical and a branched-chain alkynyl radical having one or more carbon-carbon triple bonds. Unless otherwise specified, the alkynyl group contains 2 to 30 carbon atoms and may include, but is not limited to, ethynyl, 2-propynyl, etc. Additionally, the alkynyl group may be optionally substituted.

As used herein, the term “cycloalkynyl group” refers to a cyclic alkynyl radical. Unless otherwise specified, the cycloalkynyl group contains 3 to 20 carbon atoms. Additionally, the cycloalkynyl group may be optionally substituted.

As used herein, the term “aralkyl group” or “arylalkyl group” is used interchangeably, and refers to an alkyl group having an aromatic group as a substituent. Additionally, the aralkyl (arylalkyl) group may be optionally substituted.

As used herein, the term “aryl group” or “aromatic group” is used interchangeably, and the aryl group includes both monocyclic and fused ring groups. The fused ring may include two or more rings, wherein two carbon atoms are shared between two adjacent rings. It may also include structures in which two or more rings are simply attached to each other or fused together. Unless otherwise specified, the aryl group contains 6 to 30 carbon atoms, and may include, but is not limited to, phenyl, naphthyl, anthracenyl, phenanthrenyl, fluorenyl, dimethylfluorenyl, diphenylfluorenyl, spirofluorenyl, etc. Additionally, the aryl group may be optionally substituted.

As used herein, the term “heteroaryl group” or “heteroaromatic group” is used interchangeably, and the heteroaryl group includes both monocyclic and fused ring groups. The fused ring may include two or more rings, wherein two carbon or heteroatom atoms are shared between two adjacent rings. It may also include structures in which two or more rings are simply attached to each other or fused together. Unless otherwise specified, the heteroaryl group may contain 1 to 30 carbon atoms, and when the number of carbon atoms is one or two, ring may be formed by including additional heteroatoms. In addition, the heteroaryl group may contain 1 to 30 carbon atoms, wherein one or more carbon atoms in the ring are substituted with heteroatoms such as oxygen (O), nitrogen (N), sulfur (S), or selenium (Se). The heteroaryl group may include, but is not limited to, a 6-membered monocyclic ring such as pyridinyl, pyrazinyl, pyrimidinyl, pyridazinyl, or triazinyl, a polycyclic ring such as phenoxathiinyl, indolizinyl, indolyl, purinyl, quinolyl, isoquinolyl, benzoxyzolyl, benzothiazolyl, dibenzoxyzolyl, dibenzothiazolyl, benzimidazolyl, benzofuranyl, dibenzofuranyl, benzothiophenyl, dibenzothiophenyl, phenylcarbazolyl, 9-phenylcarbazolyl, or carbazolyl, and 2-furanyl, N-imidazolyl, 2-isoxazolyl, 2-pyridyl, 2-pyrimidyl, etc. Additionally, the heteroaryl group may be optionally substituted.

As used herein, the term “heterocyclic group” refers to a group in which one or more carbon atoms of an aryl group, a cycloalkyl group, a cycloalkenyl group, a cycloalkynyl group, an arylalkyl group, or an arylamino group are substituted with heteroatoms such as oxygen (O), nitrogen (N), sulfur (S), or selenium (Se). Based on the above definition, the heterocyclic group includes a heteroaryl group, a heterocycloalkyl group, a heterocycloalkenyl group, a heterocycloalkynyl group, a heteroarylalkyl group, and a heteroarylamino group. Additionally, the heterocyclic group may be optionally substituted.

As used herein, the term “carbon ring” may refer to both a “cycloalkyl group,” which is an alicyclic ring group, and an “aryl group (aromatic group),” which is an aromatic ring group, unless otherwise specified.

As used herein, the terms “heteroalkyl group” and “heteroarylalkyl group” refer to an alkyl group or an arylalkyl group in which one or more carbon atoms are substituted with heteroatoms such as oxygen (O), nitrogen (N), sulfur (S), or selenium (Se). Additionally, the heteroalkyl group and the heteroarylalkyl group may be optionally substituted.

As used herein, the terms “alkylamino group,” “arylalkylamino group,” “arylamino group,” and “heteroarylamino group” refer to an amino group (or amine group) in which at least one hydrogen atom of is substituted with an alkyl group, an arylalkyl group, an aryl group, or an heteroaryl group, and include all primary, secondary, and tertiary amino (or amine) groups. Additionally, the alkylamino group, the arylalkylamino group, the arylamino group, and the heteroarylamino group may be optionally substituted.

As used herein, the terms “alkylsilyl group,” “arylsilyl group,” “alkoxy group,” “aryloxy group,” “alkylthio group,” and “arylthio group” refer to a silyl group, an oxy group, and a thio group substituted with the alkyl group and the aryl group as described above. Additionally, the alkylsilyl group, the arylsilyl group, the alkoxy group, the aryloxy group, the alkylthio group, and arylthio group may be optionally substituted.

As used herein, the terms “arylene group,” “arylalkylene group,” “heteroarylene group,” and “heteroarylalkylene group” refer to divalent substituents of the respective aryl group, arylalkyl group, heteroaryl group, and heteroarylalkyl group, each of which contains at least one additional substitution. Additionally, the arylene group, the arylalkylene group, the heteroarylene group, and the heteroarylalkylene group may be optionally substituted.

As used herein, the term “substitution” refers to the replacement of a hydrogen (H) atom bonded to a carbon atom, a nitrogen atom, etc., of the compound of the present disclosure with a substituent other than hydrogen. When a plurality of substituents are present, the substituents may be identical to or different from each other.

The substituents may each independently be selected from the group consisting of deuterium, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, a heteroaryl group having 1 to 30 carbon atoms, an amine group, an alkylamino group having 1 to 10 carbon atoms, an arylamino group having 6 to 30 carbon atoms, a heteroarylamino group having 1 to 30 carbon atoms, a silyl group, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms.

Each target and substituent defined herein may be identical to or different from each other, unless otherwise specified.

The units used herein are based on weight (wt), unless otherwise specified. For example, when “%” is indicated, it is to be interpreted as weight percent (wt %).

Hereinafter, an organic compound according to the present disclosure and an organic light emitting diode comprising the same will be described in detail.

According to an embodiment of the present disclosure, L₁ may be selected form a single bond and a substituted or unsubstituted arylene group having 6 to 15 carbon atoms. For example, L₁ may be selected form a single bond and a substituted or unsubstituted phenylene group.

According to an embodiment of the present disclosure, Ar₁ may be selected from a substituted or unsubstituted aryl group having 6 to 30 carbon atoms, and a substituted or unsubstituted heteroaryl group having 1 to 30 carbon atoms and containing at least one heteroatom selected from the group consisting of oxygen (O) and sulfur (S). For example, Ar₁ may be selected from the group consisting of a substituted or unsubstituted phenyl group, a substituted or unsubstituted biphenyl group, a substituted or unsubstituted naphthyl group, a substituted or unsubstituted phenanthrenyl group, a substituted or unsubstituted dibenzofuranyl group, a substituted or unsubstituted dibenzothiophenyl group, and a substituted or unsubstituted dimethylfluorenyl group.

According to an embodiment of the present disclosure, Ar₁ may be a substituted or unsubstituted aryl group having 6 to 30 carbon atoms. For example, Ar₁ may be a substituted or unsubstituted phenyl group, a substituted or unsubstituted biphenyl group, a substituted or unsubstituted naphthyl group, a substituted or unsubstituted phenanthrenyl group, or a substituted or unsubstituted dimethylfluorenyl group. When Ar₁ is selected from the above, it is more suitable as a material for the hole transport auxiliary layer.

According to an embodiment of the present disclosure, Ar₂ may be a substituted or unsubstituted aryl group having 6 to 15 carbon atoms. For example, Ar₂ may be a substituted or unsubstituted phenyl group, a substituted or unsubstituted naphthyl group, and a substituted or unsubstituted phenanthrenyl group.

According to an embodiment of the present disclosure, each substituent in L₁, Ar₁, Ar₂, and R₁ to R₂₅ is identical to or different from each other, and may each independently be one or more selected from the group consisting of deuterium, methyl, ethyl, propyl, butyl, pentyl, hexyl, cyclopropyl, cyclobutyl, cyclopentyl, cyclohexyl, adamantyl, phenyl, biphenyl, naphthyl, phenyl-naphthyl, anthracenyl, phenanthrenyl, dimethylfluorenyl, diphenylfluorenyl, spirofluorenyl, benzofuranyl, benzothiophenyl, dibenzofuranyl, dibenzothiophenyl, carbazolyl, phenylcarbazolyl, and 9-phenylcarbazolyl.

According to an embodiment of the present disclosure, Ar₂ may be any one of the following Chemical Formulas 2 to 5:

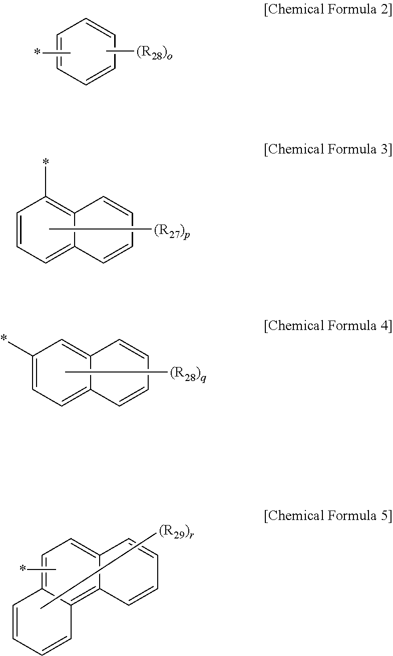

wherein in the chemical formulas 2 to 5,
R₂₆ to R₂₉ are identical to or different from each other, and are each independently selected from the group consisting of hydrogen, deuterium, a substituted or unsubstituted aryl group having 6 to 30 carbon atoms, and a substituted or unsubstituted heteroaryl group having 1 to 30 carbon atoms;
o is an integer from 1 to 5, each of p and q is an integer from 1 to 7, and r is an integer from 1 to 9; and
* denotes a bonding position.

According to an embodiment of the present disclosure, when Ar₂ is selected from one of Chemical Formulas 2 to 4, Ar₁ may be selected from a substituted or unsubstituted aryl group having 6 to 30 carbon atoms, and a substituted or unsubstituted aryl group having 6 to 15 carbon atoms. For example, Ar₁ may be selected from a substituted or unsubstituted phenyl group, a substituted or unsubstituted biphenyl group, a substituted or unsubstituted naphthyl group, a substituted or unsubstituted phenanthrenyl group, and a substituted or unsubstituted dimethylfluorenyl group.

According to an embodiment of the present disclosure, when Ar₂ is selected from Chemical Formula 5, Ar₁ may be selected from a substituted or unsubstituted aryl group having 6 to 30 carbon atoms, and a substituted or unsubstituted heteroaryl group having 1 to 30 carbon atoms. For example, Ar₁ may be selected from the group consisting of a substituted or unsubstituted phenyl group, a substituted or unsubstituted biphenyl group, a substituted or unsubstituted naphthyl group, a substituted or unsubstituted phenanthrenyl group, a substituted or unsubstituted dibenzofuranyl group, a substituted or unsubstituted dibenzothiophenyl group, and a substituted or unsubstituted dimethylfluorenyl group.

According to an embodiment of the present disclosure, the Chemical Formula 1 may be represented by any one of the following Chemical Formulas 6 to 8:

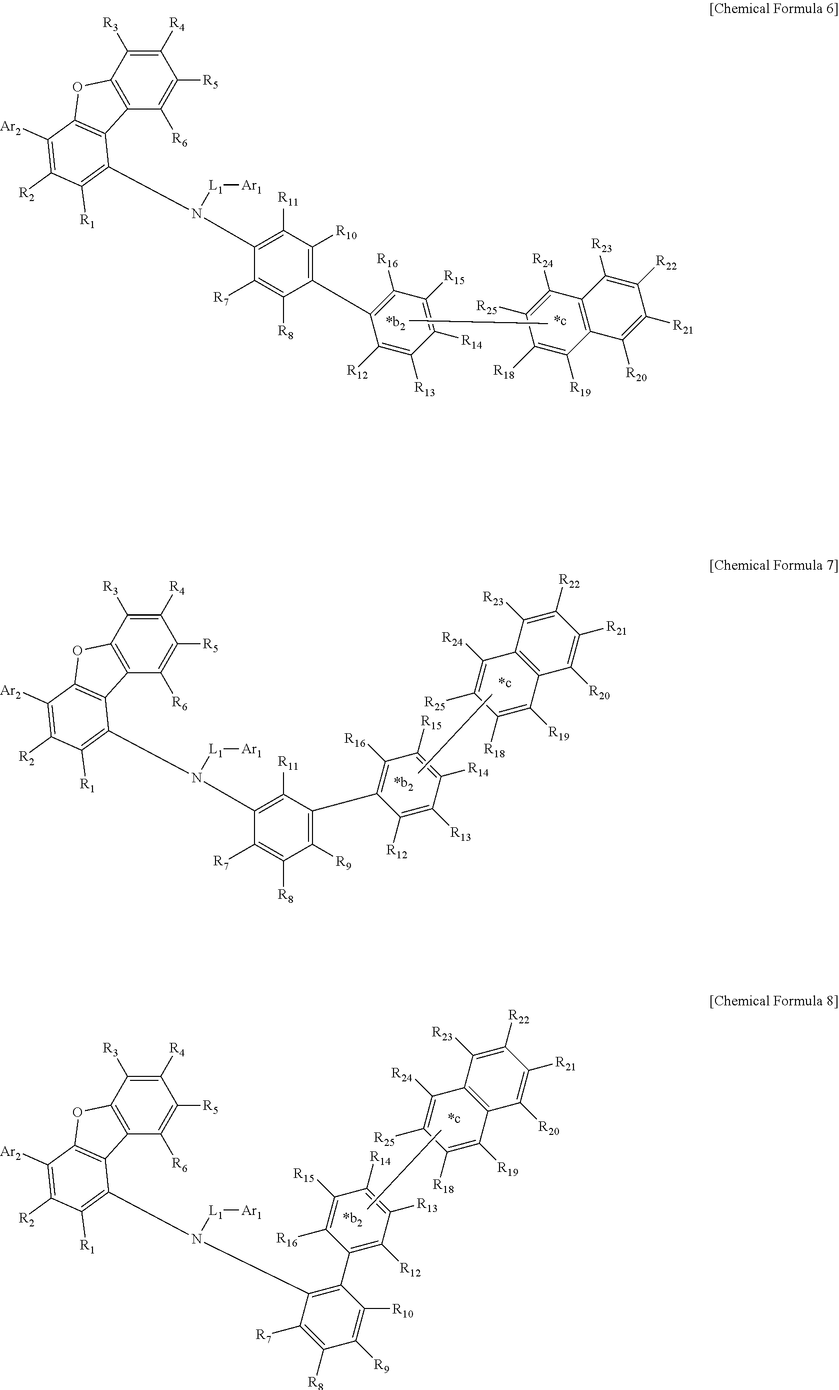

wherein in the chemical formulas 6 to 8,
L₁, Ar₁, Ar₂, and R₁ to R₂₅, and the definitions of the substituents thereof are as defined in the Chemical Formula 1.
*b₂ represents a bonding position on a phenylene moiety, and *c represents a bonding position on a naphthyl moiety;
one of R₁₂ to R₁₆ is a single bond that is boned to *b₂; and
one of R₁₈ to R₂₅ is a single bond that is boned to *c.

According to an embodiment of the present disclosure, the Chemical Formula 1 may be represented by any one of the following Chemical Formulas 9 to 17:

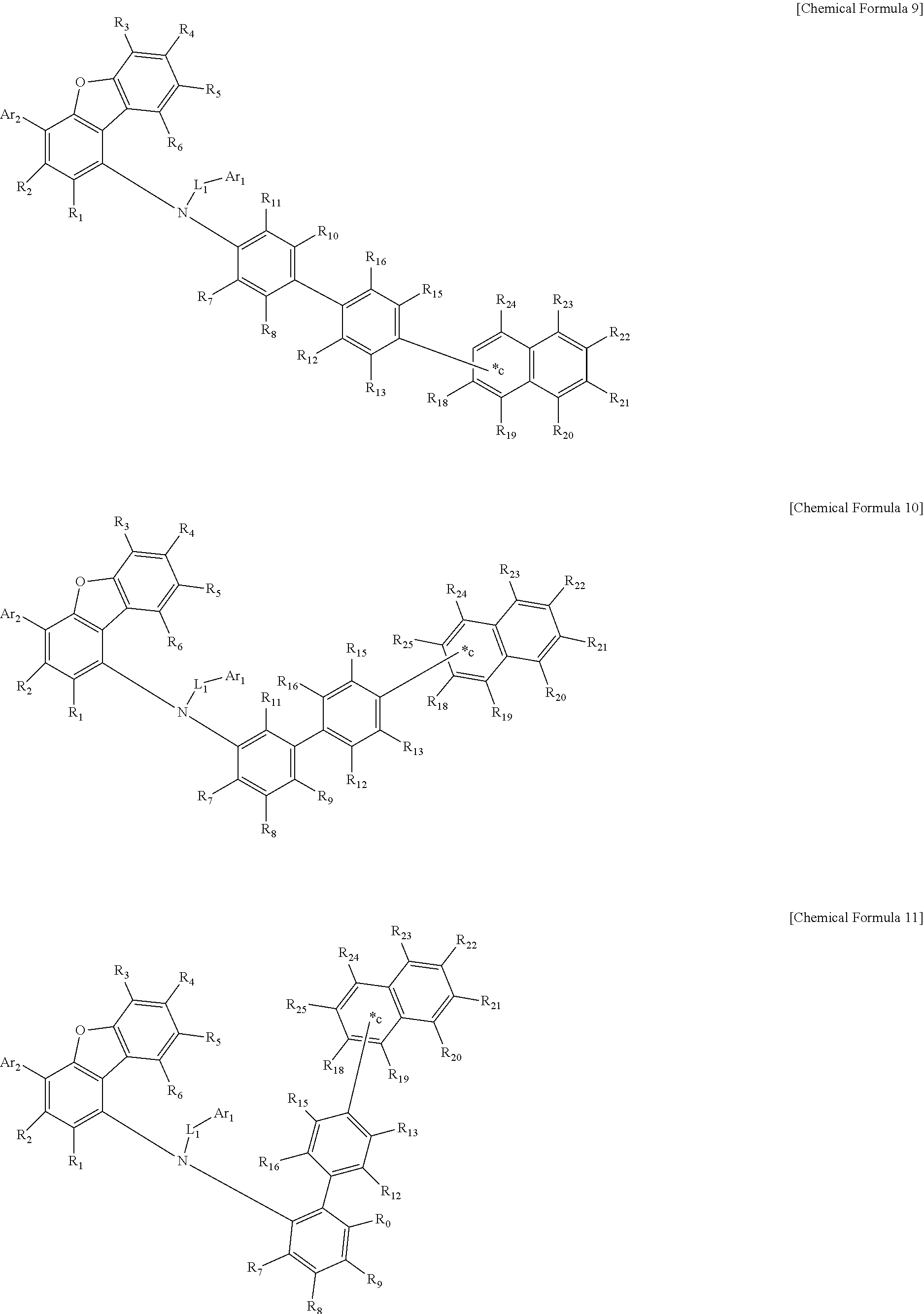

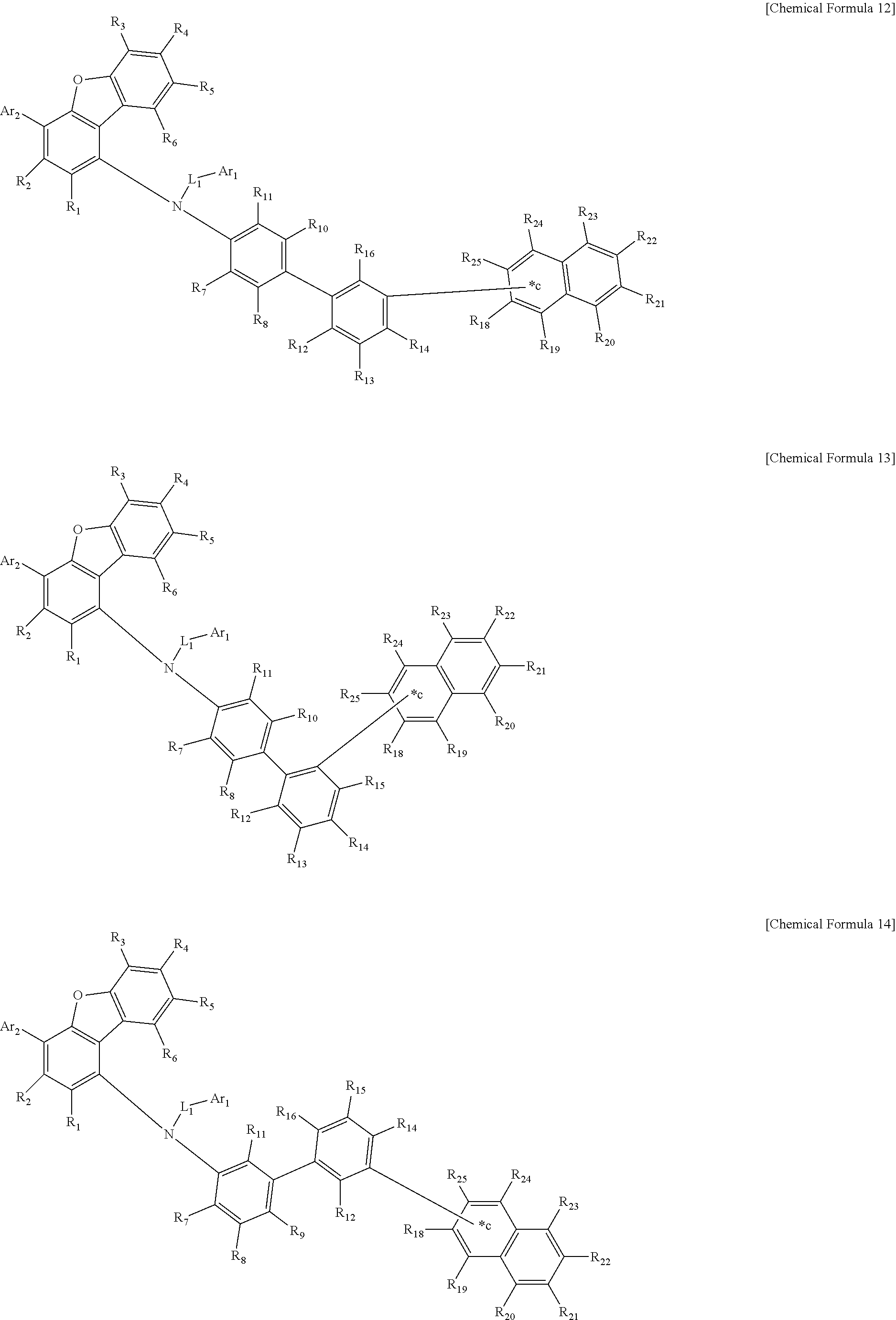

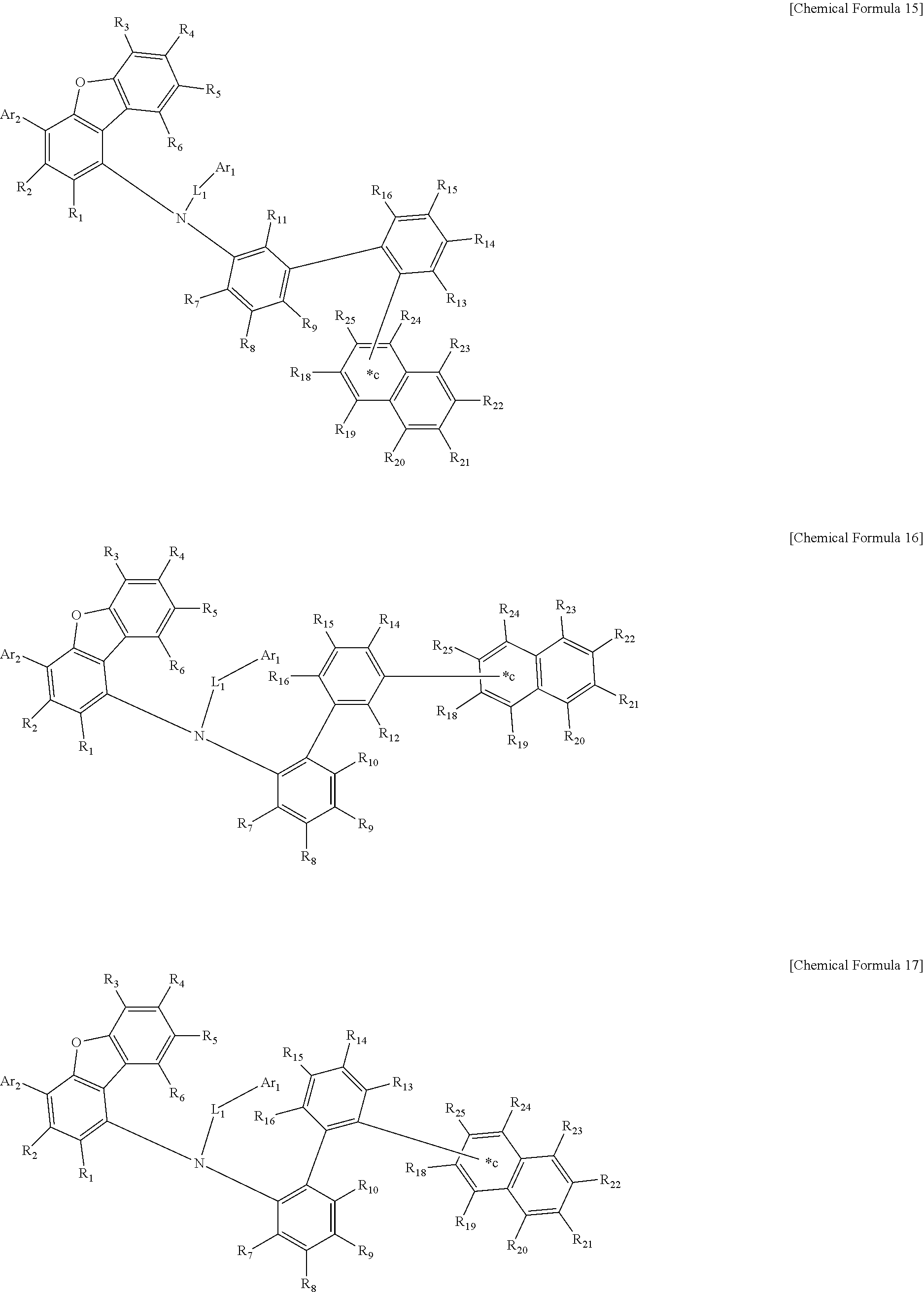

wherein in the chemical formulas 9 to 17,
L₁, Ar₁, Ar₂, and R₁ to R₂₅, and the definitions of the substituents thereof are as defined in the Chemical Formula 1.
*c represents a bonding position on a naphthyl moiety, and one of R₁₈ to R₂₅ is a single bond that is bonded to the *c.

According to an embodiment of the present disclosure, Ar₁ may be any one of the following Chemical Formulas 18 to 21:

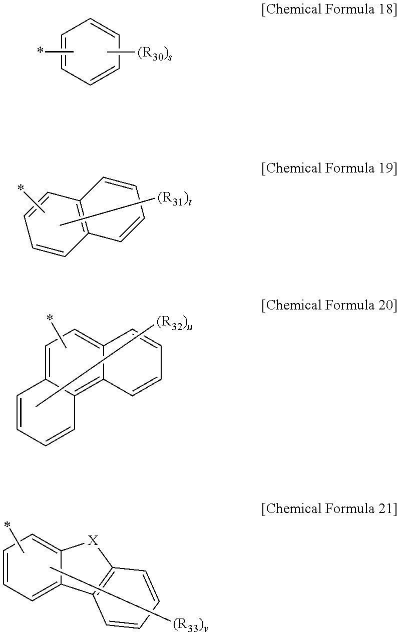

wherein R₃₀ to R₃₃ are identical to or different from each other, and are each independently selected from the group consisting of hydrogen, deuterium, a substituted or unsubstituted aryl group having 6 to 30 carbon atoms, and a substituted or unsubstituted heteroaryl group having 1 to 30 carbon atoms;
s is an integer from 1 to 5, each of t and v is integer from 1 to 7, and u is an integer from 1 to 9.

In the Chemical Formula 21, X may be selected from CR₃₄R₃₅, O, and S.

According to an embodiment of the present disclosure, when Ar₂ is selected from any one of Chemical Formulas 2 to 4, X may be CR₃₄R₃₅.

According to an embodiment of the present disclosure, when Ar₂ is selected form Chemical Formula 5, X may be selected from CR₃₄R₃₅, O, and S.

R₃₄ and R₃₅ are identical to or different from each other, and are each independently selected from the group consisting of hydrogen, deuterium, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms.

* denotes a bonding position.

According to an embodiment of the present disclosure, the organic compound represented by Chemical Formula 1 has a tertiary amine structure of the type NRR′R″, wherein R is a dibenzofuran group as defined below and is connected to nitrogen (N) at the 1-position, R′ is an aryl group in the form of a substituted or unsubstituted phenylene-phenylene-naphthyl, and R″ is L₁-Ar₁ connected to nitrogen (N). The organic compound represented by Chemical Formula 1 according to the present disclosure has a structure in which a substituted or unsubstituted aryl group is connected to the 4-position (Ar₂) of the dibenzofuran group of R, thereby increasing conjugation and expanding the electron cloud of the highest occupied molecular orbital (HOMO), which can lead to increased hole injection and hole transport properties. In addition, it may have an energy level suitable for serving as a hole transport auxiliary layer that transfers holes from the hole transport layer to the emitting layer and blocks electrons coming from the emitting layer, thereby exhibiting properties suitable as a material for the hole transport auxiliary layer.

In addition, by having an asymmetric structure centered on the arylamine moiety, the HOMO-LUMO bandgap may be readily adjusted, and the crystallinity of the molecule may be reduced due to the asymmetry. Such low crystallinity not only facilitates purification of the compound to obtain a high-purity product, but also lowers the risk of clogging caused by material condensation at the inlet of the container during OLED manufacturing processes such as deposition.

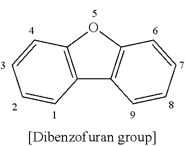 [SMILES: c1ccc2c(c1)oc1ccccc12]

According to an embodiment of the present disclosure, the organic compound represented by Chemical Formula 1 may be represented by any one of Chemical Formulas 9 to 12, wherein Ar₂ is selected from a substituted or unsubstituted phenyl group, a substituted or unsubstituted naphthyl group, and a substituted or unsubstituted phenanthrene group. When Ar₁ is connected to nitrogen (N) through L₁, the connection between L₁ and Ar₁ may be in a meta, para, or ortho position, or may be in a meta or para position, and when the connection is in the meta position, it is advantageous in that the performance of the organic light emitting diode may be further improved.

According to another embodiment of the present disclosure, the compound of the present disclosure is represented by any one of Chemical Formulas 9 to 12, wherein Ar₂ is selected from a substituted or unsubstituted phenyl group, a substituted or unsubstituted naphthyl group, and a substituted or unsubstituted phenanthrene group, and L₁ is present such that L₁ and Ar₁ are connected in a meta, para, or ortho position, or in a meta or para position, or in the meta position, and when Ar₁ is a substituted or unsubstituted aryl group having 6 to 30 carbon atoms, the performance of the organic light emitting diode may be further improved.

According to still another embodiment of the present disclosure, L₁ may be a single bond or may be selected from M1 to M3 below.

In M1 to M3 below, * denotes a bonding position, and Dn refers to a structure in which n hydrogen atoms are substituted with deuterium within the structures of M1 to M3 below, and n, which represents the number of deuterium atoms, is an integer of 0 or more, with the maximum value being limited by the number of hydrogen atoms in the structure that may be substituted. For example, in M1, when n is 0, it represents

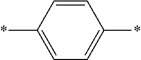 [SMILES: *c1ccc(*)cc1]

(denoted as D₀), and when n is 1, it represents

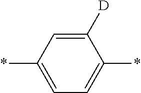 [SMILES: *c1ccc(*)c([2H])c1]

in which one of the four hydrogen atoms on the phenyl group is substituted with a deuterium (denoted as D₁).

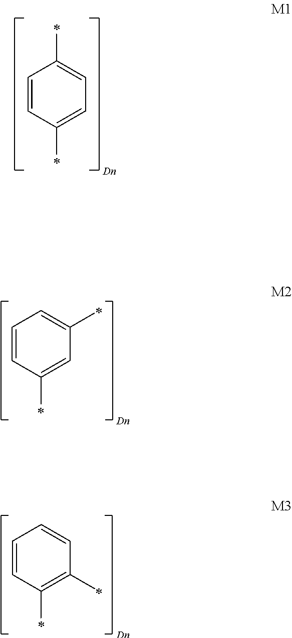

According to an embodiment of the present disclosure, the organic compound represented by Chemical Formula 1 may be selected from the group consisting of, but is not limited to, the following compounds.

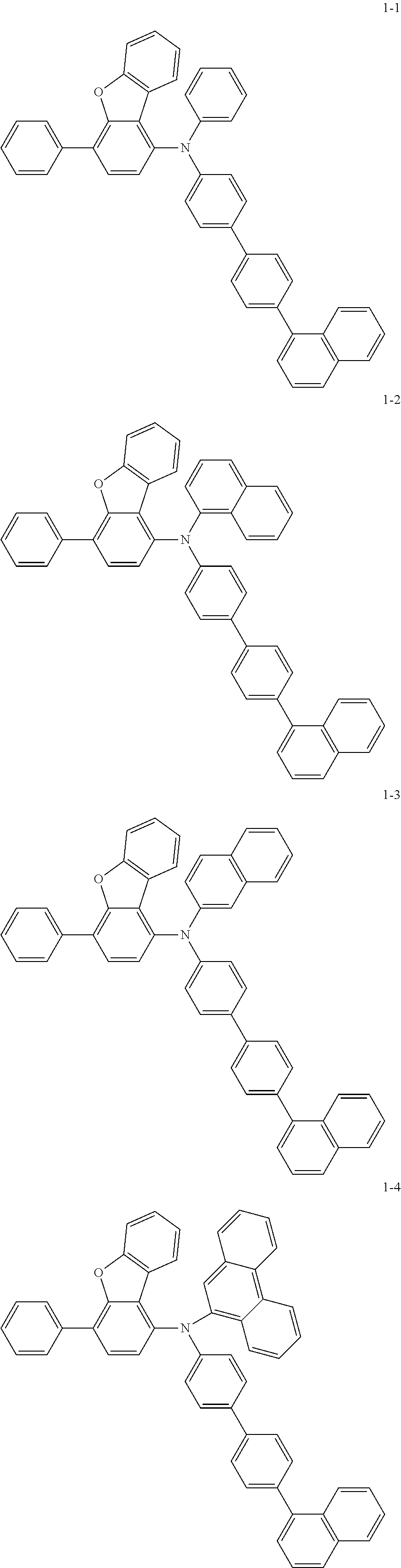 [SMILES: c1ccc(-c2ccc(N(c3ccccc3)c3ccc(-c4ccc(-c5cccc6ccccc56)cc4)cc3)c3c2oc2ccccc23)cc1 ; c1ccc(-c2ccc(N(c3ccc(-c4ccc(-c5cccc6ccccc56)cc4)cc3)c3cccc4ccccc34)c3c2oc2ccccc23)cc1 ; c1ccc(-c2ccc(N(c3ccc(-c4ccc(-c5cccc6ccccc56)cc4)cc3)c3ccc4ccccc4c3)c3c2oc2ccccc23)cc1 ; c1ccc(-c2ccc(N(c3ccc(-c4ccc(-c5cccc6ccccc56)cc4)cc3)c3cc4ccccc4c4ccccc34)c3c2oc2ccccc23)cc1]

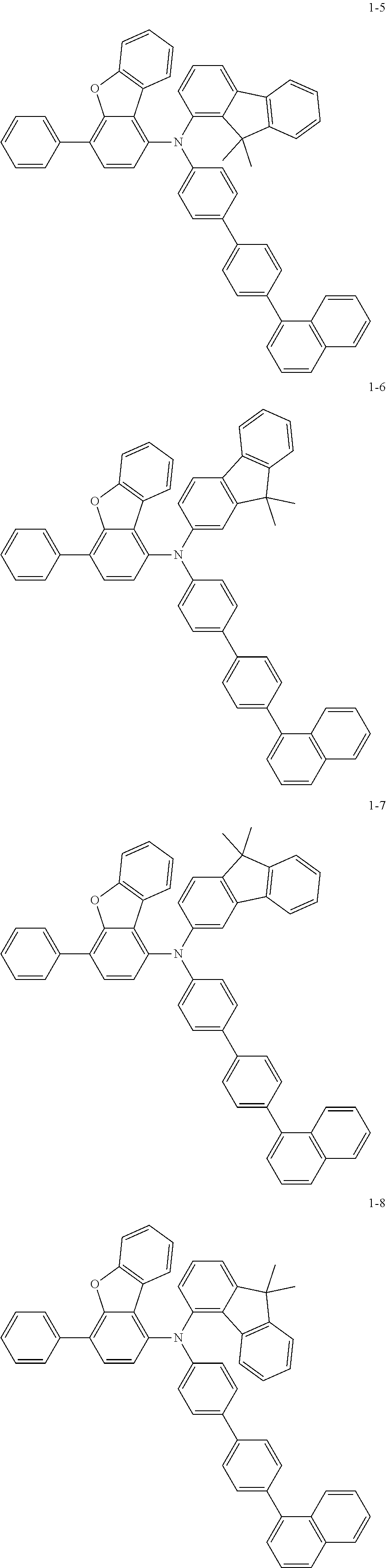 [SMILES: CC1(C)c2ccccc2-c2cccc(N(c3ccc(-c4ccc(-c5cccc6ccccc56)cc4)cc3)c3ccc(-c4ccccc4)c4oc5ccccc5c34)c21 ; CC1(C)c2ccccc2-c2ccc(N(c3ccc(-c4ccc(-c5cccc6ccccc56)cc4)cc3)c3ccc(-c4ccccc4)c4oc5ccccc5c34)cc21 ; CC1(C)c2ccccc2-c2cc(N(c3ccc(-c4ccc(-c5cccc6ccccc56)cc4)cc3)c3ccc(-c4ccccc4)c4oc5ccccc5c34)ccc21 ; CC1(C)c2ccccc2-c2c(N(c3ccc(-c4ccc(-c5cccc6ccccc56)cc4)cc3)c3ccc(-c4ccccc4)c4oc5ccccc5c34)cccc21]

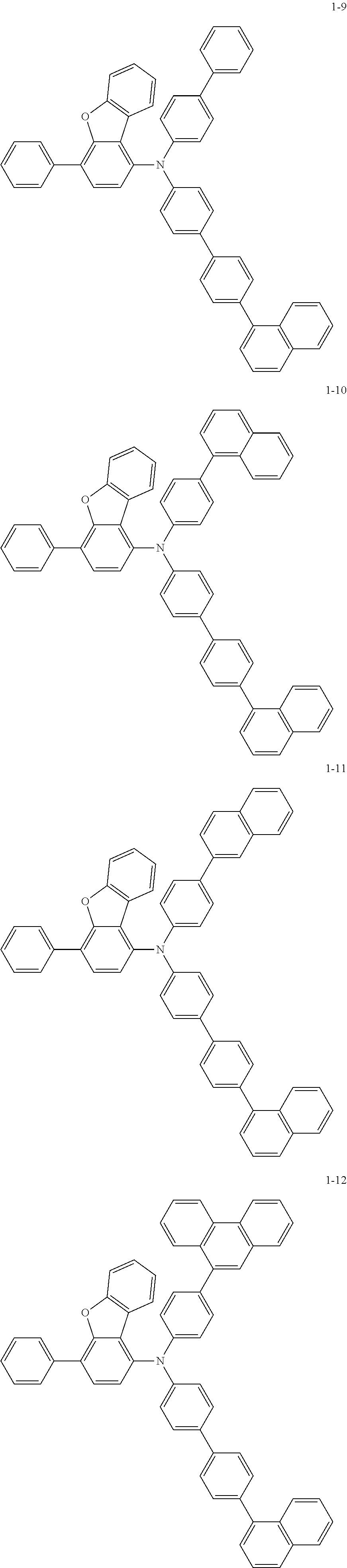

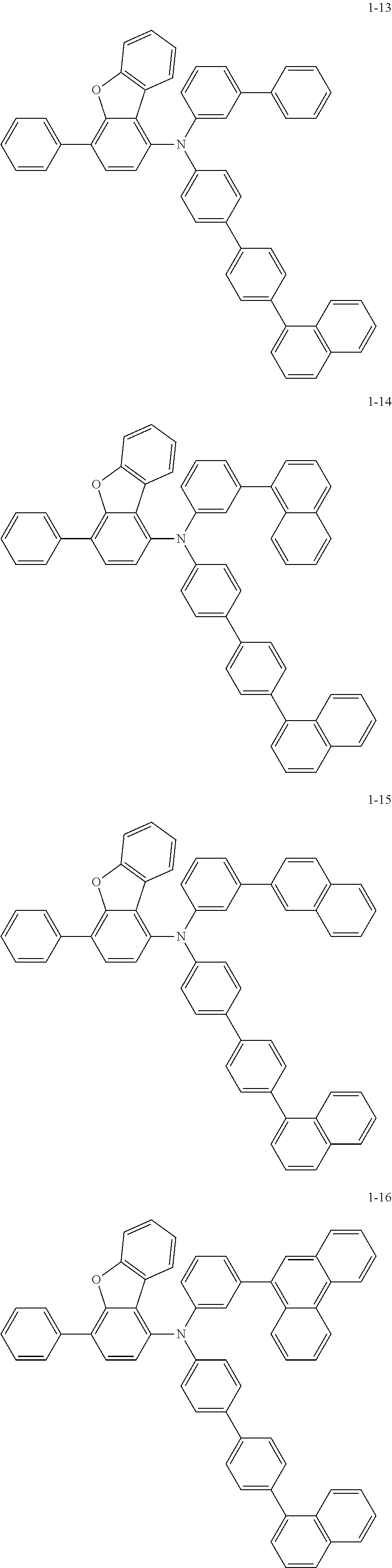 [SMILES: c1ccc(-c2cccc(N(c3ccc(-c4ccc(-c5cccc6ccccc56)cc4)cc3)c3ccc(-c4ccccc4)c4oc5ccccc5c34)c2)cc1 ; c1ccc(-c2ccc(N(c3ccc(-c4ccc(-c5cccc6ccccc56)cc4)cc3)c3cccc(-c4cccc5ccccc45)c3)c3c2oc2ccccc23)cc1 ; c1ccc(-c2ccc(N(c3ccc(-c4ccc(-c5cccc6ccccc56)cc4)cc3)c3cccc(-c4ccc5ccccc5c4)c3)c3c2oc2ccccc23)cc1 ; c1ccc(-c2ccc(N(c3ccc(-c4ccc(-c5cccc6ccccc56)cc4)cc3)c3cccc(-c4cc5ccccc5c5ccccc45)c3)c3c2oc2ccccc23)cc1]

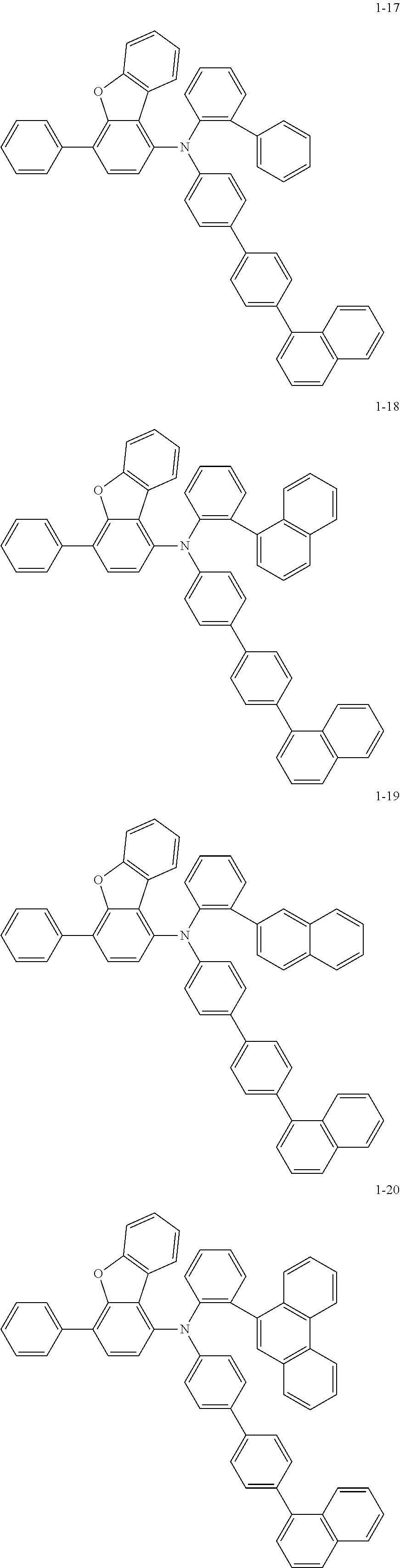 [SMILES: c1ccc(-c2ccccc2N(c2ccc(-c3ccc(-c4cccc5ccccc45)cc3)cc2)c2ccc(-c3ccccc3)c3oc4ccccc4c23)cc1 ; c1ccc(-c2ccc(N(c3ccc(-c4ccc(-c5cccc6ccccc56)cc4)cc3)c3ccccc3-c3cccc4ccccc34)c3c2oc2ccccc23)cc1 ; c1ccc(-c2ccc(N(c3ccc(-c4ccc(-c5cccc6ccccc56)cc4)cc3)c3ccccc3-c3ccc4ccccc4c3)c3c2oc2ccccc23)cc1 ; c1ccc(-c2ccc(N(c3ccc(-c4ccc(-c5cccc6ccccc56)cc4)cc3)c3ccccc3-c3cc4ccccc4c4ccccc34)c3c2oc2ccccc23)cc1]

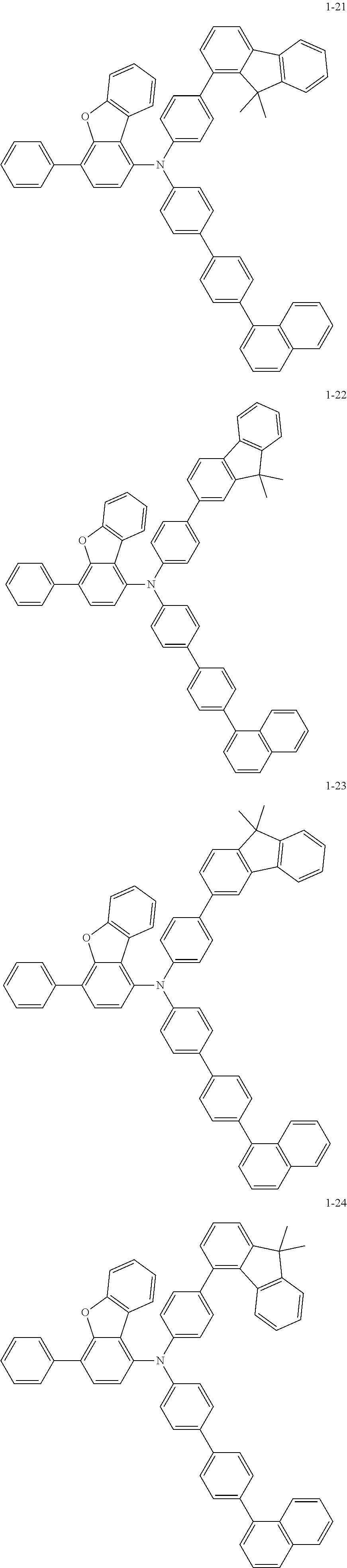

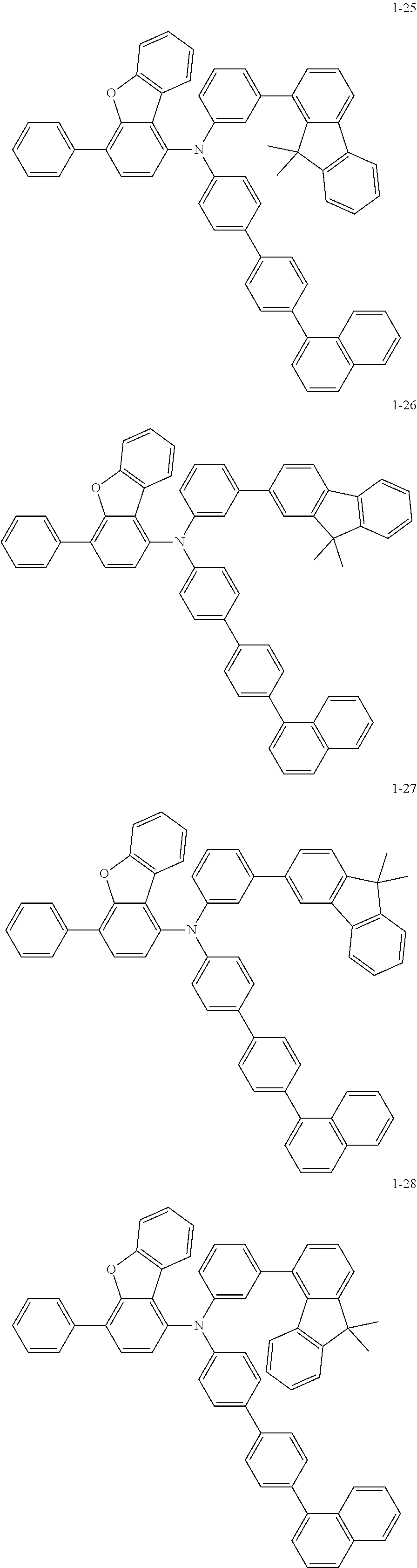 [SMILES: CC1(C)c2ccccc2-c2cccc(-c3cccc(N(c4ccc(-c5ccc(-c6cccc7ccccc67)cc5)cc4)c4ccc(-c5ccccc5)c5oc6ccccc6c45)c3)c21 ; CC1(C)c2ccccc2-c2ccc(-c3cccc(N(c4ccc(-c5ccc(-c6cccc7ccccc67)cc5)cc4)c4ccc(-c5ccccc5)c5oc6ccccc6c45)c3)cc21 ; CC1(C)c2ccccc2-c2cc(-c3cccc(N(c4ccc(-c5ccc(-c6cccc7ccccc67)cc5)cc4)c4ccc(-c5ccccc5)c5oc6ccccc6c45)c3)ccc21 ; CC1(C)c2ccccc2-c2c(-c3cccc(N(c4ccc(-c5ccc(-c6cccc7ccccc67)cc5)cc4)c4ccc(-c5ccccc5)c5oc6ccccc6c45)c3)cccc21]

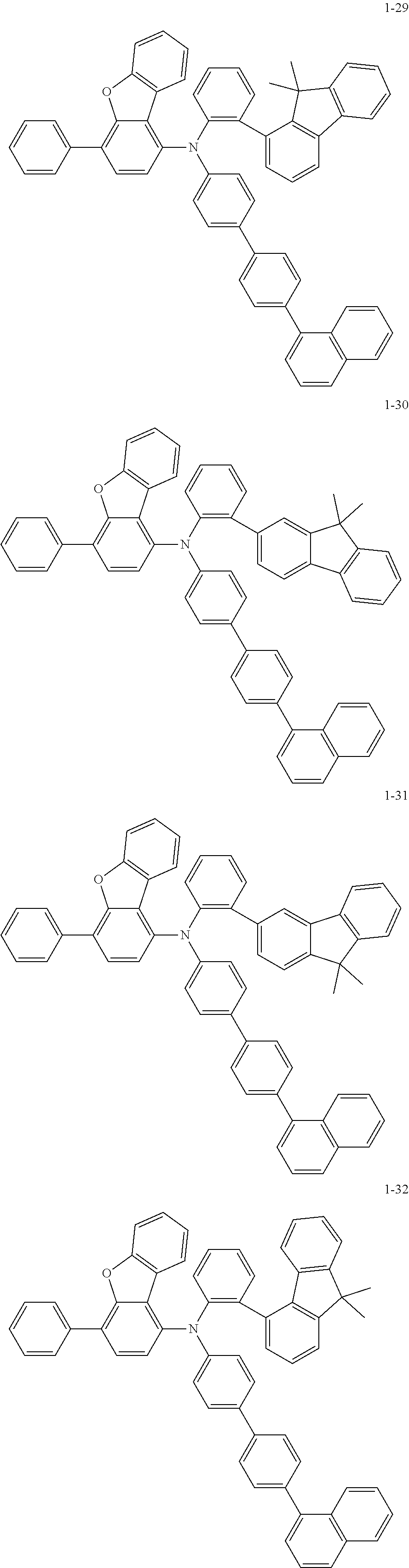 [SMILES: CC1(C)c2ccccc2-c2cccc(-c3ccccc3N(c3ccc(-c4ccc(-c5cccc6ccccc56)cc4)cc3)c3ccc(-c4ccccc4)c4oc5ccccc5c34)c21 ; CC1(C)c2ccccc2-c2ccc(-c3ccccc3N(c3ccc(-c4ccc(-c5cccc6ccccc56)cc4)cc3)c3ccc(-c4ccccc4)c4oc5ccccc5c34)cc21 ; CC1(C)c2ccccc2-c2cc(-c3ccccc3N(c3ccc(-c4ccc(-c5cccc6ccccc56)cc4)cc3)c3ccc(-c4ccccc4)c4oc5ccccc5c34)ccc21 ; CC1(C)c2ccccc2-c2c(-c3ccccc3N(c3ccc(-c4ccc(-c5cccc6ccccc56)cc4)cc3)c3ccc(-c4ccccc4)c4oc5ccccc5c34)cccc21]

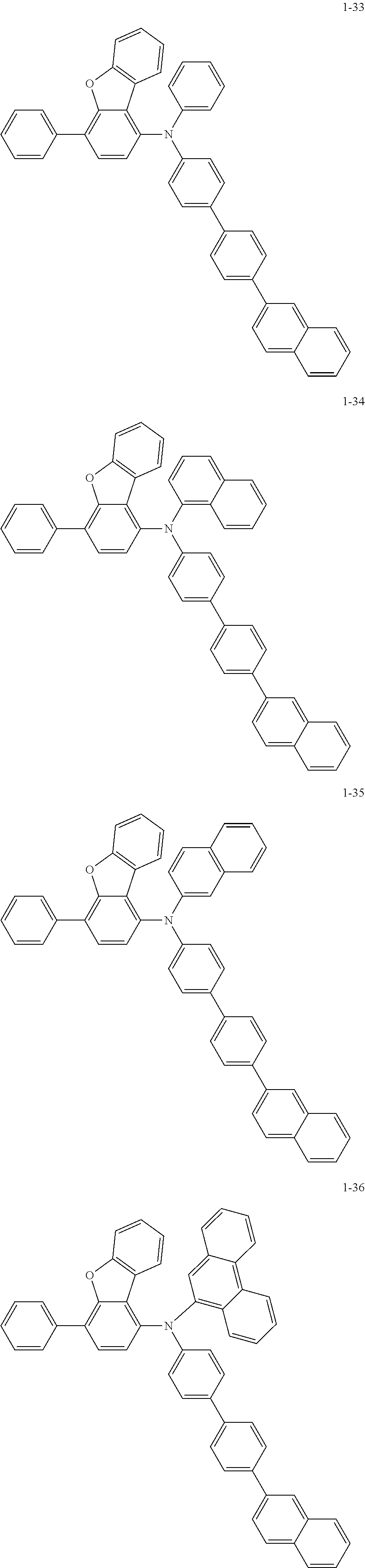 [SMILES: c1ccc(-c2ccc(N(c3ccccc3)c3ccc(-c4ccc(-c5ccc6ccccc6c5)cc4)cc3)c3c2oc2ccccc23)cc1 ; c1ccc(-c2ccc(N(c3ccc(-c4ccc(-c5ccc6ccccc6c5)cc4)cc3)c3cccc4ccccc34)c3c2oc2ccccc23)cc1 ; c1ccc(-c2ccc(N(c3ccc(-c4ccc(-c5ccc6ccccc6c5)cc4)cc3)c3ccc4ccccc4c3)c3c2oc2ccccc23)cc1 ; c1ccc(-c2ccc(N(c3ccc(-c4ccc(-c5ccc6ccccc6c5)cc4)cc3)c3cc4ccccc4c4ccccc34)c3c2oc2ccccc23)cc1]

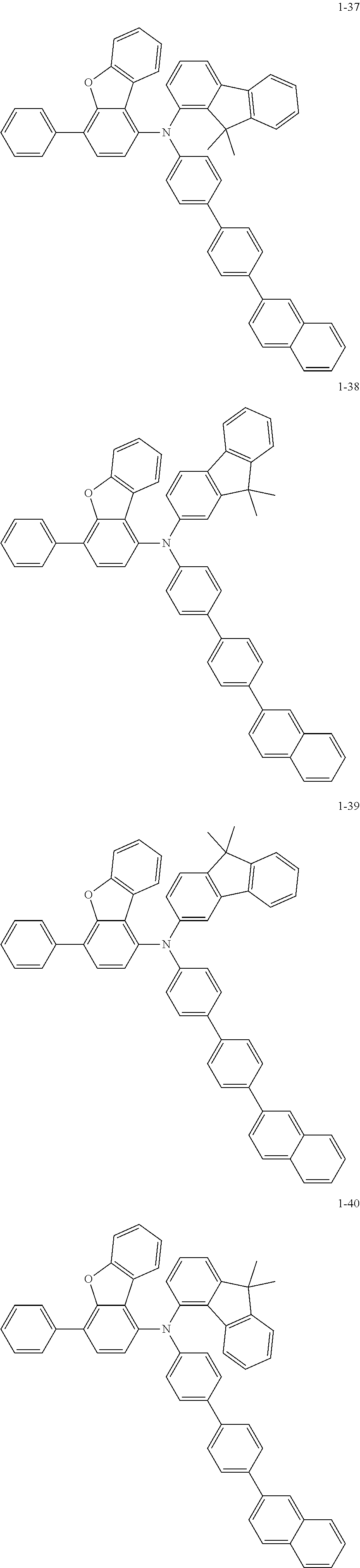 [SMILES: CC1(C)c2ccccc2-c2cccc(N(c3ccc(-c4ccc(-c5ccc6ccccc6c5)cc4)cc3)c3ccc(-c4ccccc4)c4oc5ccccc5c34)c21 ; CC1(C)c2ccccc2-c2ccc(N(c3ccc(-c4ccc(-c5ccc6ccccc6c5)cc4)cc3)c3ccc(-c4ccccc4)c4oc5ccccc5c34)cc21 ; CC1(C)c2ccccc2-c2cc(N(c3ccc(-c4ccc(-c5ccc6ccccc6c5)cc4)cc3)c3ccc(-c4ccccc4)c4oc5ccccc5c34)ccc21 ; CC1(C)c2ccccc2-c2c(N(c3ccc(-c4ccc(-c5ccc6ccccc6c5)cc4)cc3)c3ccc(-c4ccccc4)c4oc5ccccc5c34)cccc21]

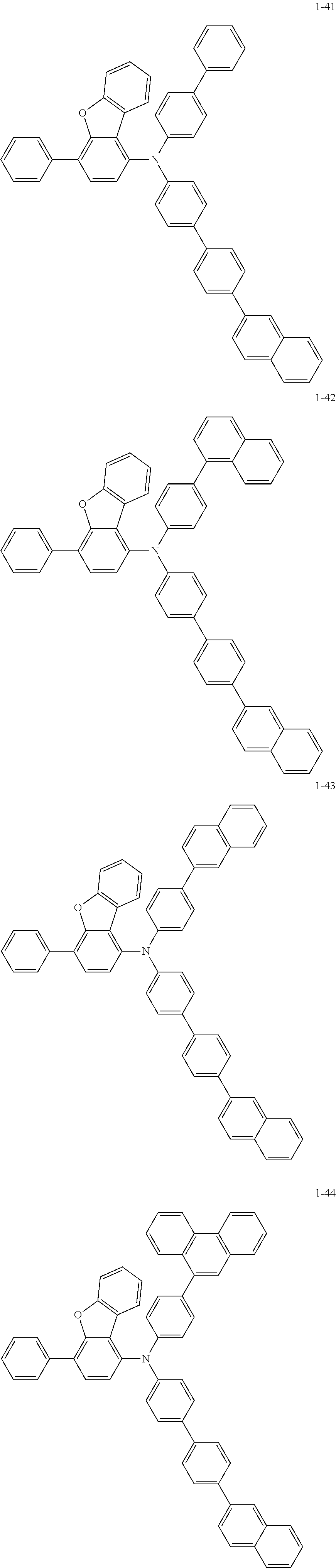 [SMILES: c1ccc(-c2ccc(N(c3ccc(-c4ccc(-c5ccc6ccccc6c5)cc4)cc3)c3ccc(-c4ccccc4)c4oc5ccccc5c34)cc2)cc1 ; c1ccc(-c2ccc(N(c3ccc(-c4ccc(-c5ccc6ccccc6c5)cc4)cc3)c3ccc(-c4cccc5ccccc45)cc3)c3c2oc2ccccc23)cc1 ; c1ccc(-c2ccc(N(c3ccc(-c4ccc(-c5ccc6ccccc6c5)cc4)cc3)c3ccc(-c4ccc5ccccc5c4)cc3)c3c2oc2ccccc23)cc1 ; c1ccc(-c2ccc(N(c3ccc(-c4ccc(-c5ccc6ccccc6c5)cc4)cc3)c3ccc(-c4cc5ccccc5c5ccccc45)cc3)c3c2oc2ccccc23)cc1]

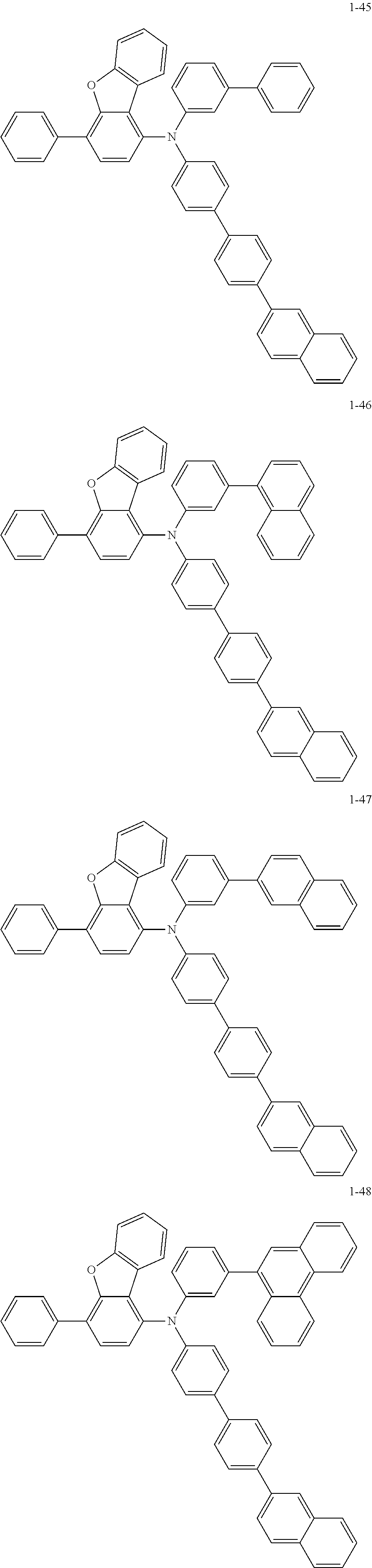 [SMILES: c1ccc(-c2cccc(N(c3ccc(-c4ccc(-c5ccc6ccccc6c5)cc4)cc3)c3ccc(-c4ccccc4)c4oc5ccccc5c34)c2)cc1 ; c1ccc(-c2ccc(N(c3ccc(-c4ccc(-c5ccc6ccccc6c5)cc4)cc3)c3cccc(-c4cccc5ccccc45)c3)c3c2oc2ccccc23)cc1 ; c1ccc(-c2ccc(N(c3ccc(-c4ccc(-c5ccc6ccccc6c5)cc4)cc3)c3cccc(-c4ccc5ccccc5c4)c3)c3c2oc2ccccc23)cc1 ; c1ccc(-c2ccc(N(c3ccc(-c4ccc(-c5ccc6ccccc6c5)cc4)cc3)c3cccc(-c4cc5ccccc5c5ccccc45)c3)c3c2oc2ccccc23)cc1]

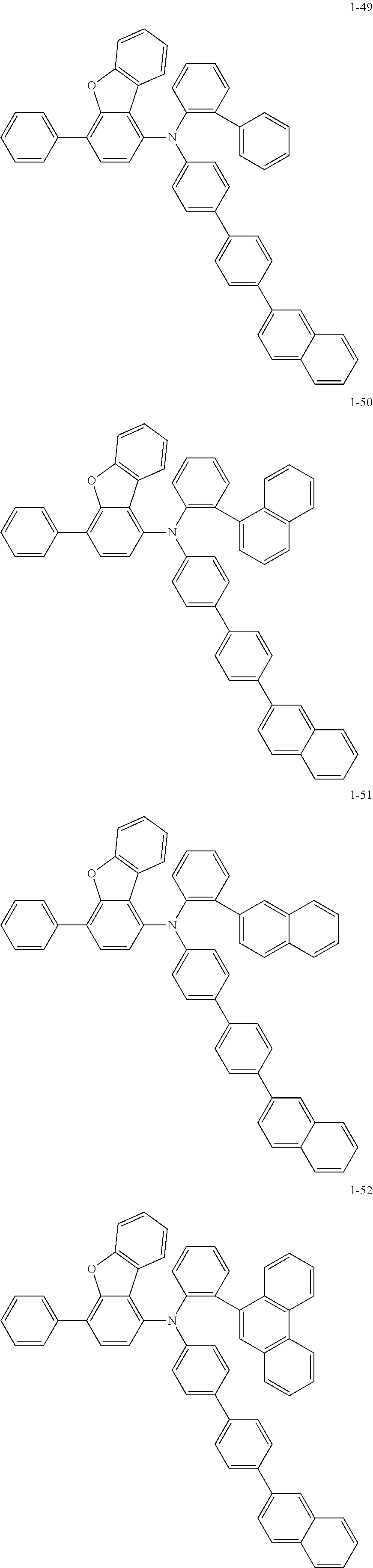 [SMILES: c1ccc(-c2ccccc2N(c2ccc(-c3ccc(-c4ccc5ccccc5c4)cc3)cc2)c2ccc(-c3ccccc3)c3oc4ccccc4c23)cc1 ; c1ccc(-c2ccc(N(c3ccc(-c4ccc(-c5ccc6ccccc6c5)cc4)cc3)c3ccccc3-c3cccc4ccccc34)c3c2oc2ccccc23)cc1 ; c1ccc(-c2ccc(N(c3ccc(-c4ccc(-c5ccc6ccccc6c5)cc4)cc3)c3ccccc3-c3ccc4ccccc4c3)c3c2oc2ccccc23)cc1 ; c1ccc(-c2ccc(N(c3ccc(-c4ccc(-c5ccc6ccccc6c5)cc4)cc3)c3ccccc3-c3cc4ccccc4c4ccccc34)c3c2oc2ccccc23)cc1]

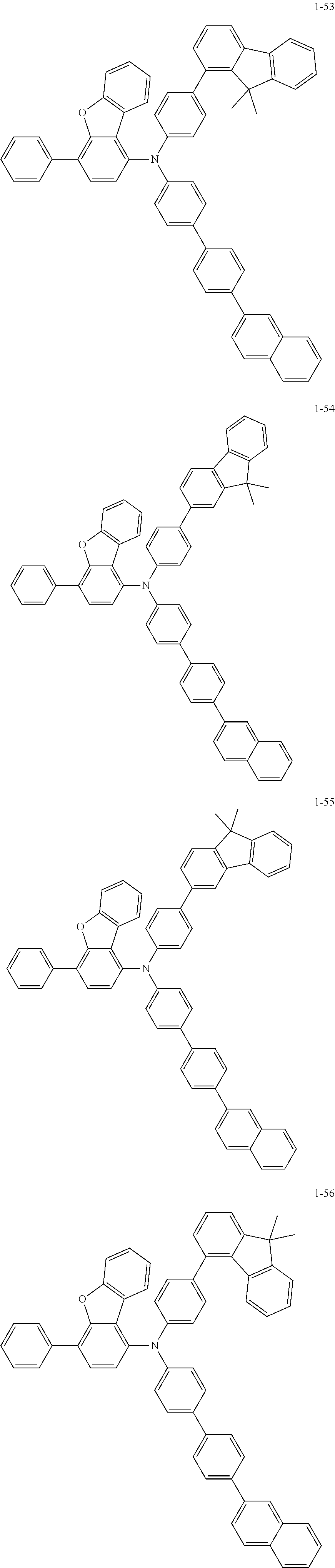 [SMILES: CC1(C)c2ccccc2-c2cccc(-c3ccc(N(c4ccc(-c5ccc(-c6ccc7ccccc7c6)cc5)cc4)c4ccc(-c5ccccc5)c5oc6ccccc6c45)cc3)c21 ; CC1(C)c2ccccc2-c2ccc(-c3ccc(N(c4ccc(-c5ccc(-c6ccc7ccccc7c6)cc5)cc4)c4ccc(-c5ccccc5)c5oc6ccccc6c45)cc3)cc21 ; CC1(C)c2ccccc2-c2cc(-c3ccc(N(c4ccc(-c5ccc(-c6ccc7ccccc7c6)cc5)cc4)c4ccc(-c5ccccc5)c5oc6ccccc6c45)cc3)ccc21 ; CC1(C)c2ccccc2-c2c(-c3ccc(N(c4ccc(-c5ccc(-c6ccc7ccccc7c6)cc5)cc4)c4ccc(-c5ccccc5)c5oc6ccccc6c45)cc3)cccc21]

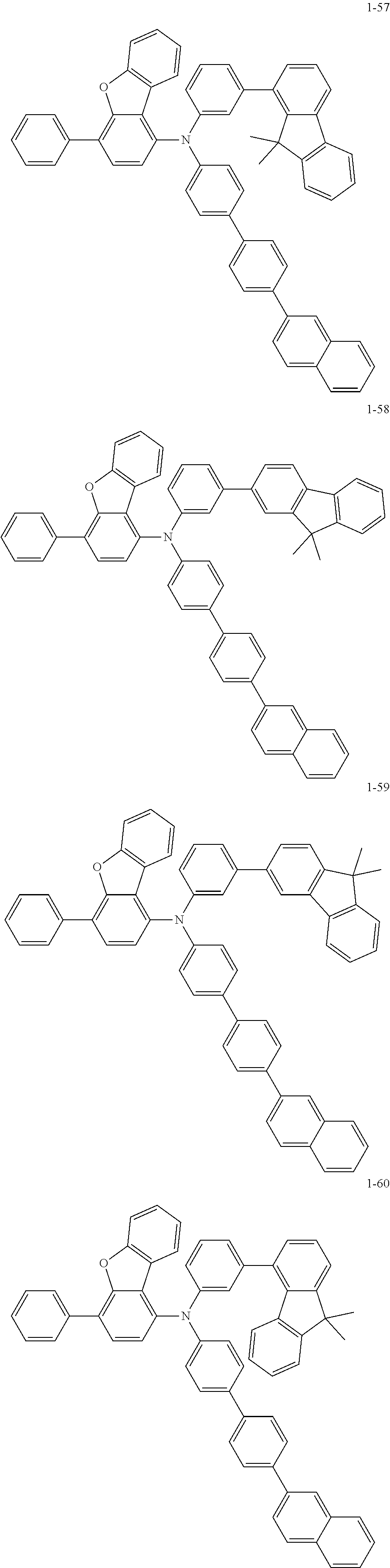 [SMILES: CC1(C)c2ccccc2-c2cccc(-c3cccc(N(c4ccc(-c5ccc(-c6ccc7ccccc7c6)cc5)cc4)c4ccc(-c5ccccc5)c5oc6ccccc6c45)c3)c21 ; CC1(C)c2ccccc2-c2ccc(-c3cccc(N(c4ccc(-c5ccc(-c6ccc7ccccc7c6)cc5)cc4)c4ccc(-c5ccccc5)c5oc6ccccc6c45)c3)cc21 ; CC1(C)c2ccccc2-c2cc(-c3cccc(N(c4ccc(-c5ccc(-c6ccc7ccccc7c6)cc5)cc4)c4ccc(-c5ccccc5)c5oc6ccccc6c45)c3)ccc21 ; CC1(C)c2ccccc2-c2c(-c3cccc(N(c4ccc(-c5ccc(-c6ccc7ccccc7c6)cc5)cc4)c4ccc(-c5ccccc5)c5oc6ccccc6c45)c3)cccc21]

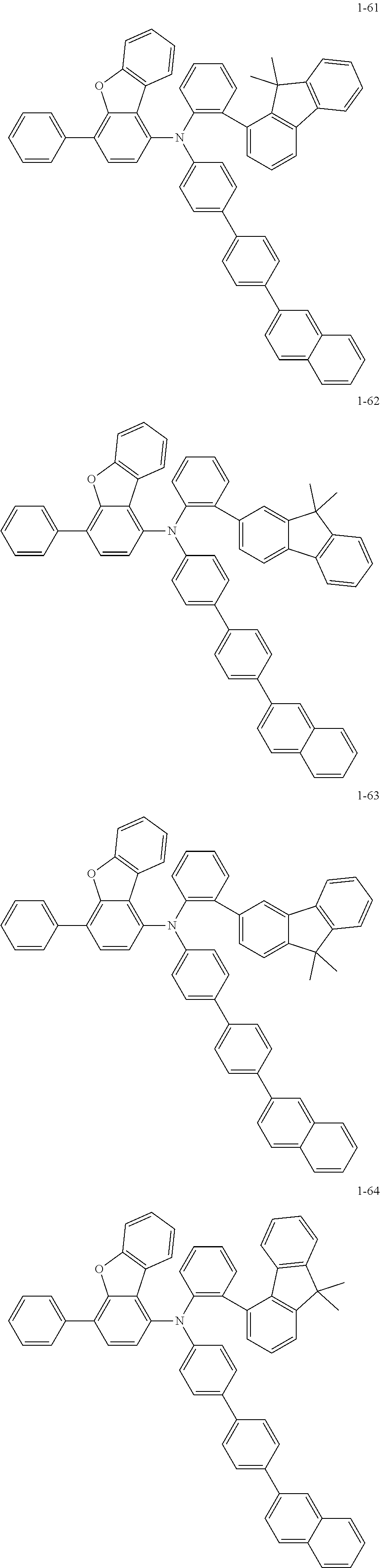 [SMILES: CC1(C)c2ccccc2-c2cccc(-c3ccccc3N(c3ccc(-c4ccc(-c5ccc6ccccc6c5)cc4)cc3)c3ccc(-c4ccccc4)c4oc5ccccc5c34)c21 ; CC1(C)c2ccccc2-c2ccc(-c3ccccc3N(c3ccc(-c4ccc(-c5ccc6ccccc6c5)cc4)cc3)c3ccc(-c4ccccc4)c4oc5ccccc5c34)cc21 ; CC1(C)c2ccccc2-c2cc(-c3ccccc3N(c3ccc(-c4ccc(-c5ccc6ccccc6c5)cc4)cc3)c3ccc(-c4ccccc4)c4oc5ccccc5c34)ccc21 ; CC1(C)c2ccccc2-c2c(-c3ccccc3N(c3ccc(-c4ccc(-c5ccc6ccccc6c5)cc4)cc3)c3ccc(-c4ccccc4)c4oc5ccccc5c34)cccc21]

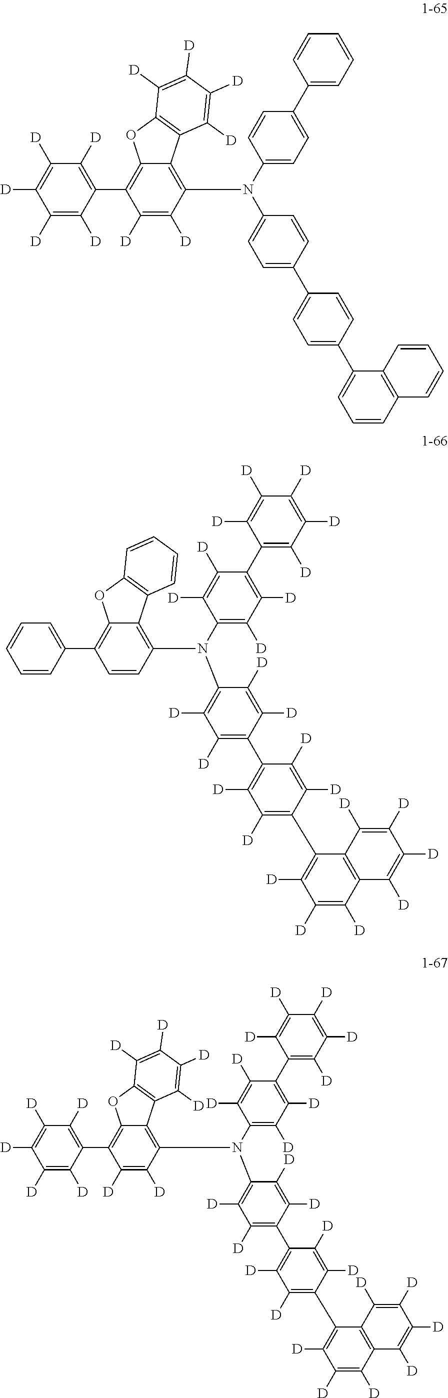

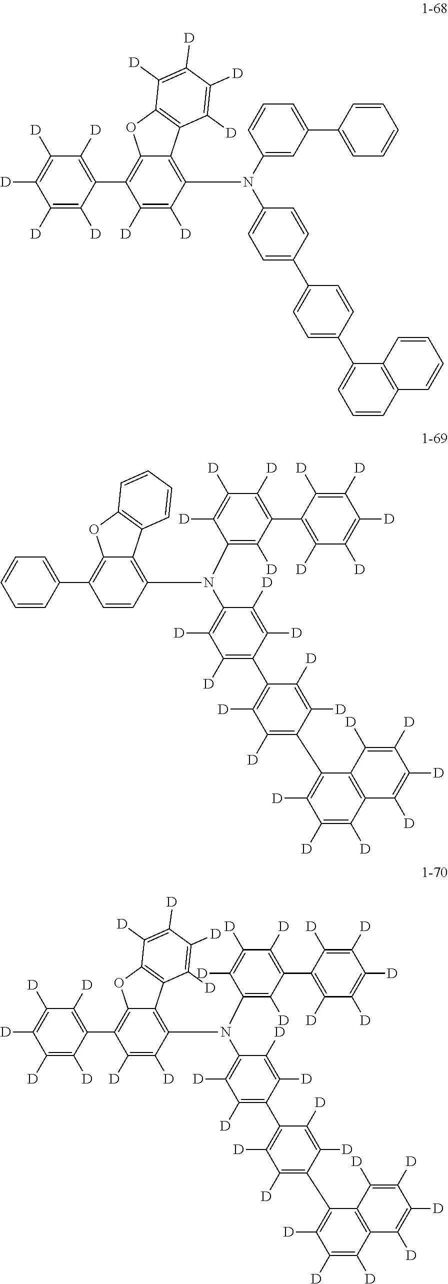

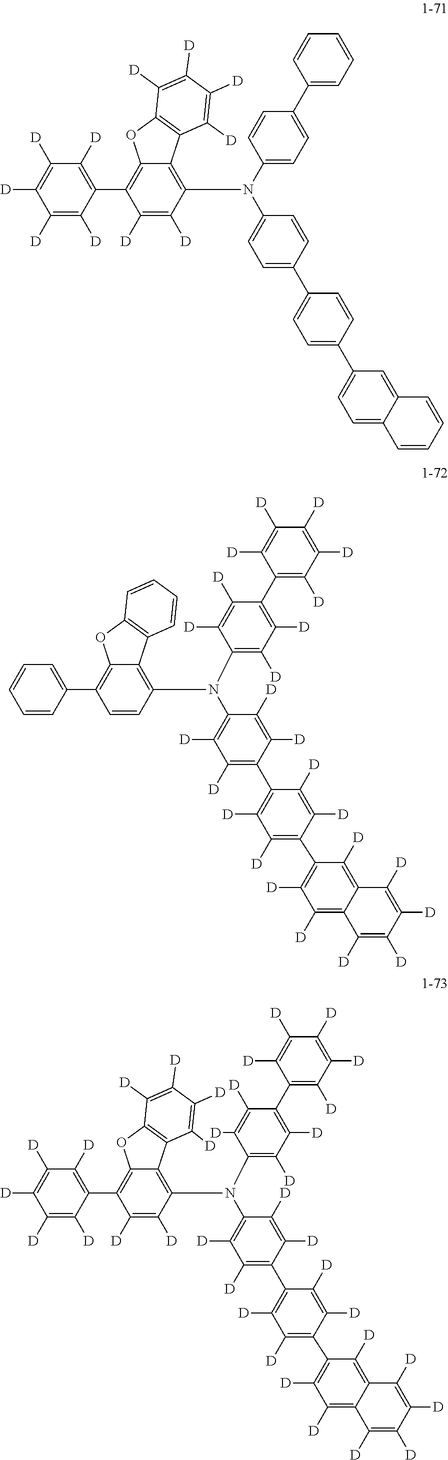

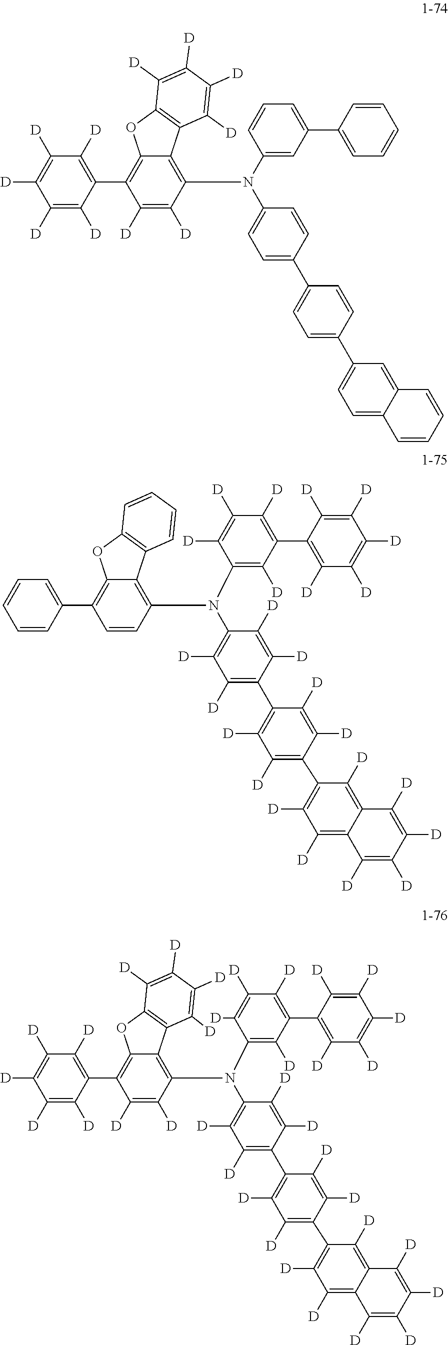

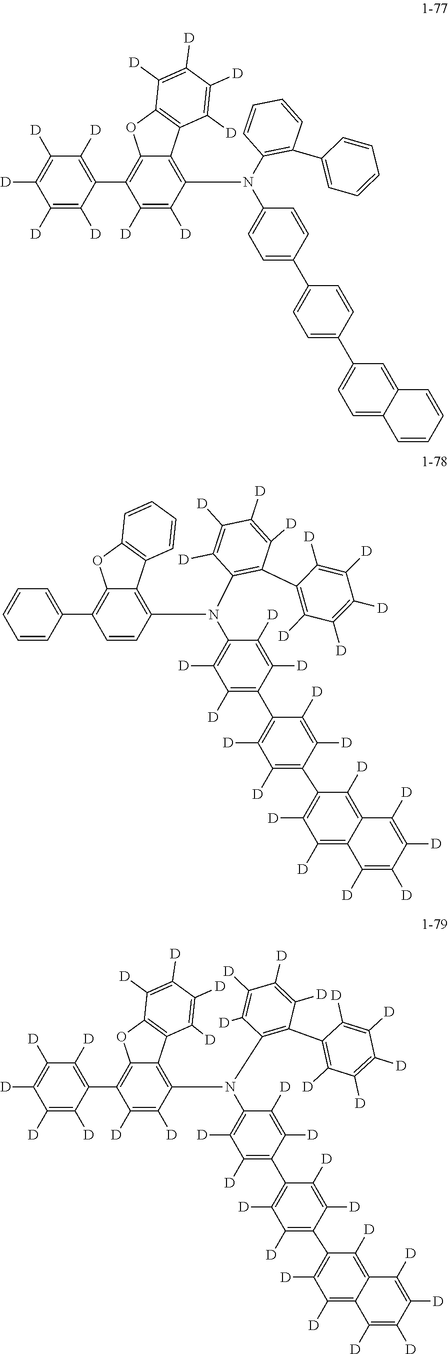

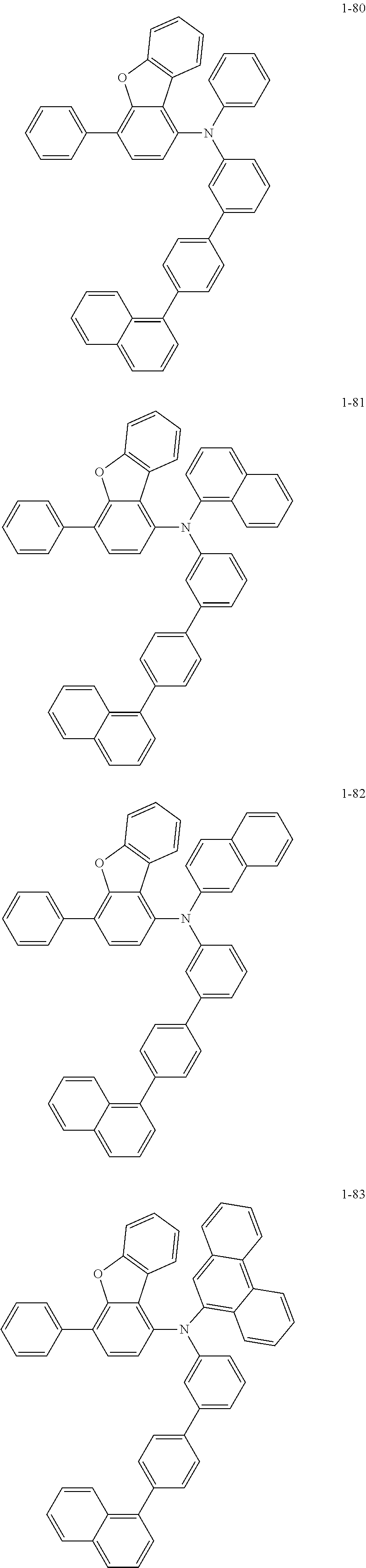

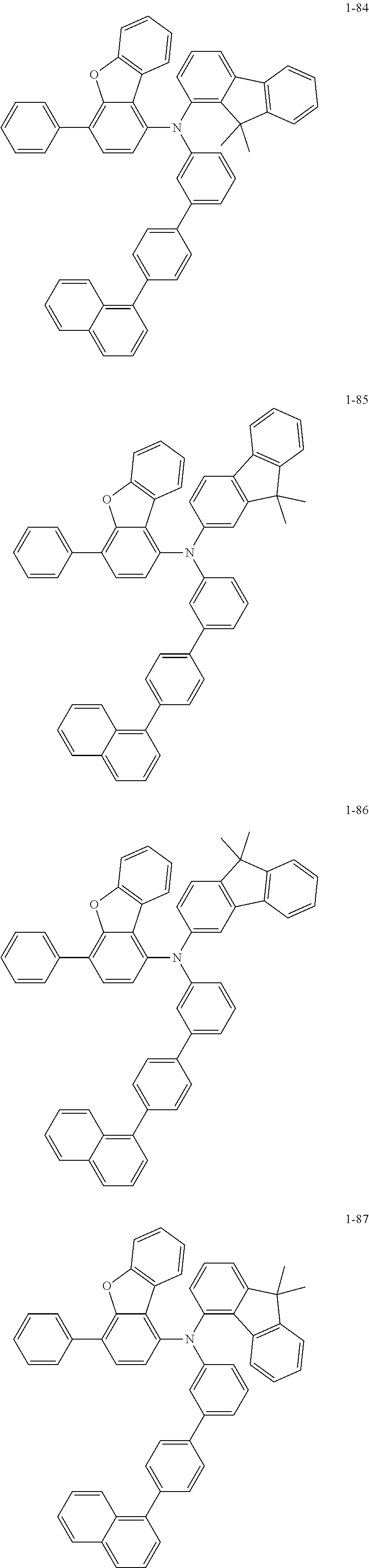 [SMILES: CC1(C)c2ccccc2-c2cccc(N(c3cccc(-c4ccc(-c5cccc6ccccc56)cc4)c3)c3ccc(-c4ccccc4)c4oc5ccccc5c34)c21 ; CC1(C)c2ccccc2-c2ccc(N(c3cccc(-c4ccc(-c5cccc6ccccc56)cc4)c3)c3ccc(-c4ccccc4)c4oc5ccccc5c34)cc21 ; CC1(C)c2ccccc2-c2cc(N(c3cccc(-c4ccc(-c5cccc6ccccc56)cc4)c3)c3ccc(-c4ccccc4)c4oc5ccccc5c34)ccc21 ; CC1(C)c2ccccc2-c2c(N(c3cccc(-c4ccc(-c5cccc6ccccc56)cc4)c3)c3ccc(-c4ccccc4)c4oc5ccccc5c34)cccc21]

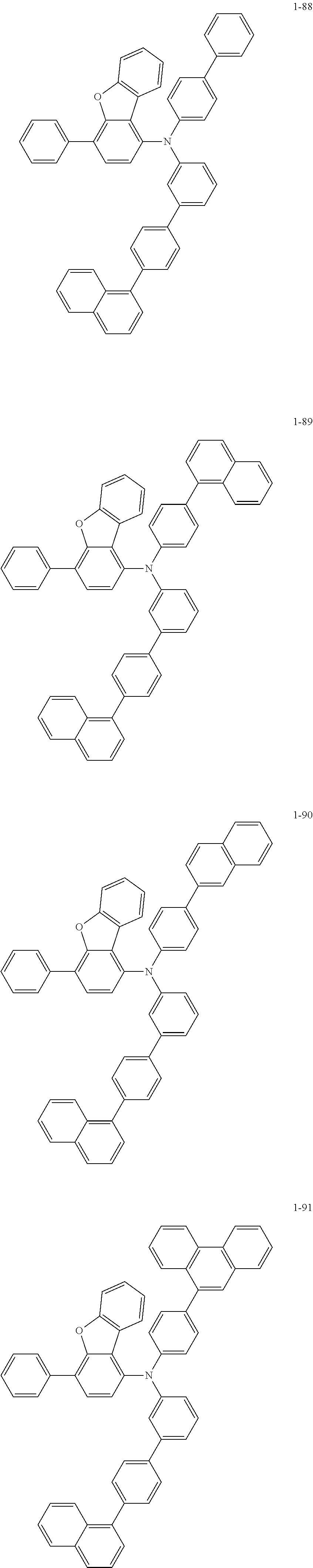 [SMILES: c1ccc(-c2ccc(N(c3cccc(-c4ccc(-c5cccc6ccccc56)cc4)c3)c3ccc(-c4ccccc4)c4oc5ccccc5c34)cc2)cc1 ; c1ccc(-c2ccc(N(c3ccc(-c4cccc5ccccc45)cc3)c3cccc(-c4ccc(-c5cccc6ccccc56)cc4)c3)c3c2oc2ccccc23)cc1 ; c1ccc(-c2ccc(N(c3ccc(-c4ccc5ccccc5c4)cc3)c3cccc(-c4ccc(-c5cccc6ccccc56)cc4)c3)c3c2oc2ccccc23)cc1 ; c1ccc(-c2ccc(N(c3ccc(-c4cc5ccccc5c5ccccc45)cc3)c3cccc(-c4ccc(-c5cccc6ccccc56)cc4)c3)c3c2oc2ccccc23)cc1]

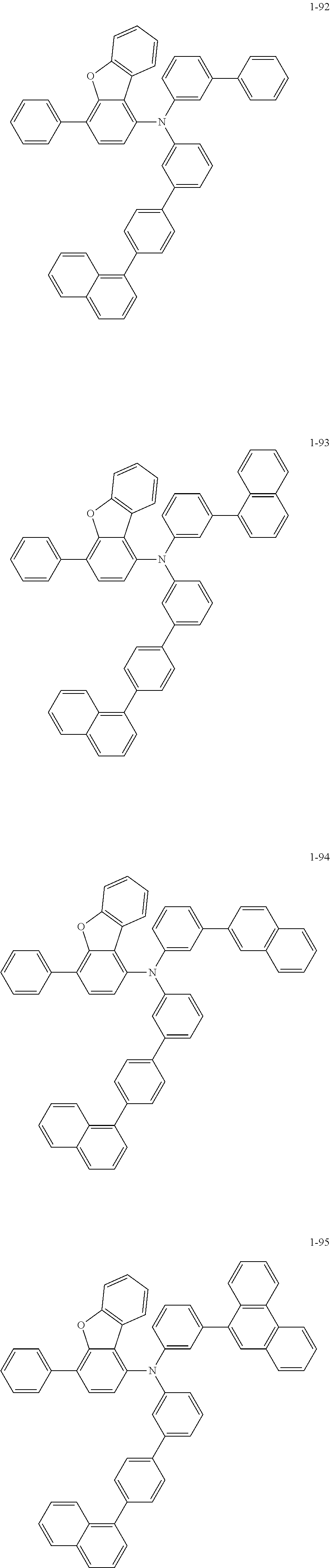 [SMILES: c1ccc(-c2cccc(N(c3cccc(-c4ccc(-c5cccc6ccccc56)cc4)c3)c3ccc(-c4ccccc4)c4oc5ccccc5c34)c2)cc1 ; c1ccc(-c2ccc(N(c3cccc(-c4ccc(-c5cccc6ccccc56)cc4)c3)c3cccc(-c4cccc5ccccc45)c3)c3c2oc2ccccc23)cc1 ; c1ccc(-c2ccc(N(c3cccc(-c4ccc(-c5cccc6ccccc56)cc4)c3)c3cccc(-c4ccc5ccccc5c4)c3)c3c2oc2ccccc23)cc1 ; c1ccc(-c2ccc(N(c3cccc(-c4ccc(-c5cccc6ccccc56)cc4)c3)c3cccc(-c4cc5ccccc5c5ccccc45)c3)c3c2oc2ccccc23)cc1]

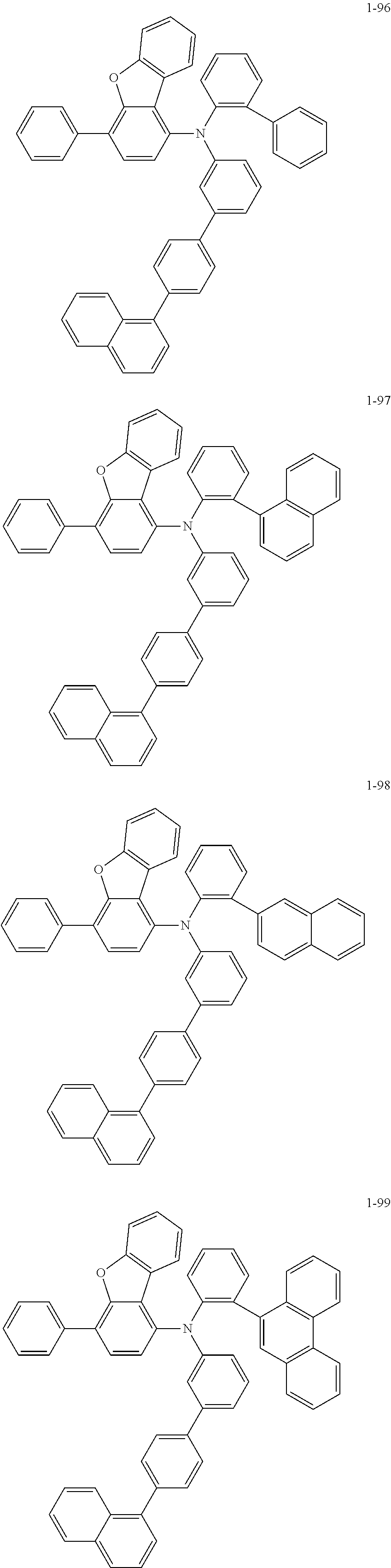

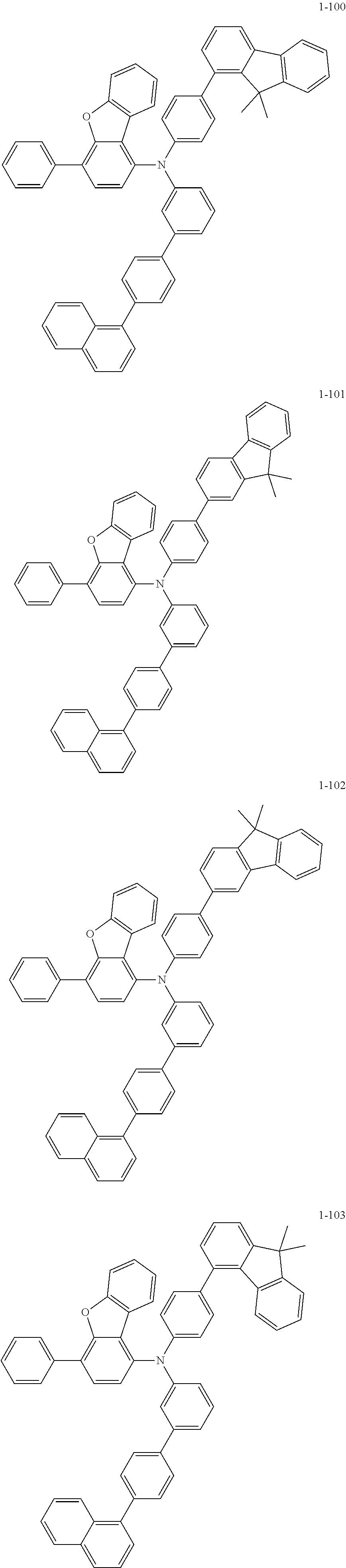 [SMILES: CC1(C)c2ccccc2-c2cccc(-c3ccc(N(c4cccc(-c5ccc(-c6cccc7ccccc67)cc5)c4)c4ccc(-c5ccccc5)c5oc6ccccc6c45)cc3)c21 ; CC1(C)c2ccccc2-c2ccc(-c3ccc(N(c4cccc(-c5ccc(-c6cccc7ccccc67)cc5)c4)c4ccc(-c5ccccc5)c5oc6ccccc6c45)cc3)cc21 ; CC1(C)c2ccccc2-c2cc(-c3ccc(N(c4cccc(-c5ccc(-c6cccc7ccccc67)cc5)c4)c4ccc(-c5ccccc5)c5oc6ccccc6c45)cc3)ccc21 ; CC1(C)c2ccccc2-c2c(-c3ccc(N(c4cccc(-c5ccc(-c6cccc7ccccc67)cc5)c4)c4ccc(-c5ccccc5)c5oc6ccccc6c45)cc3)cccc21]

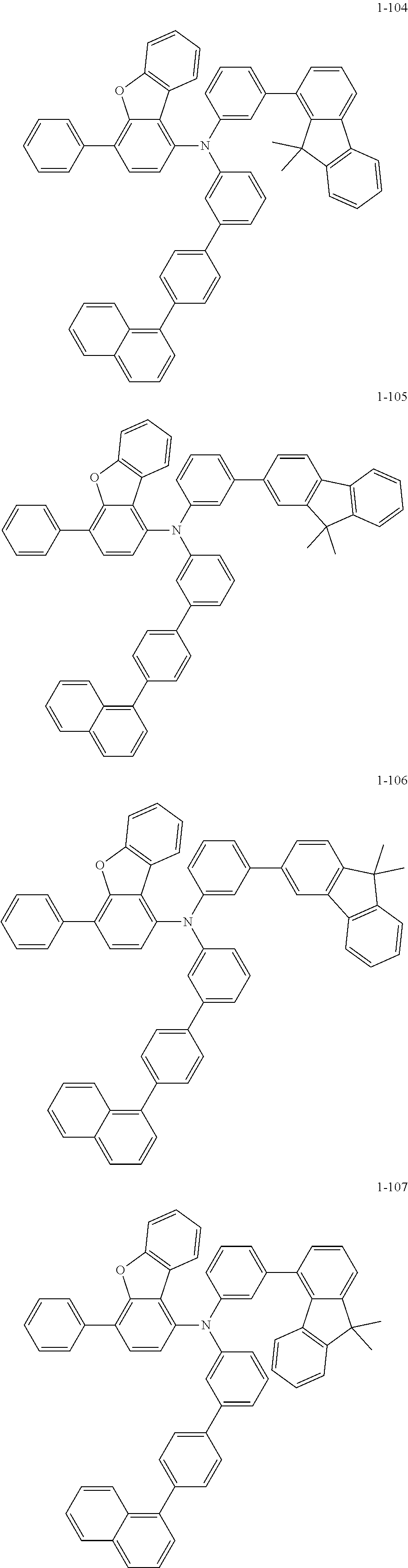

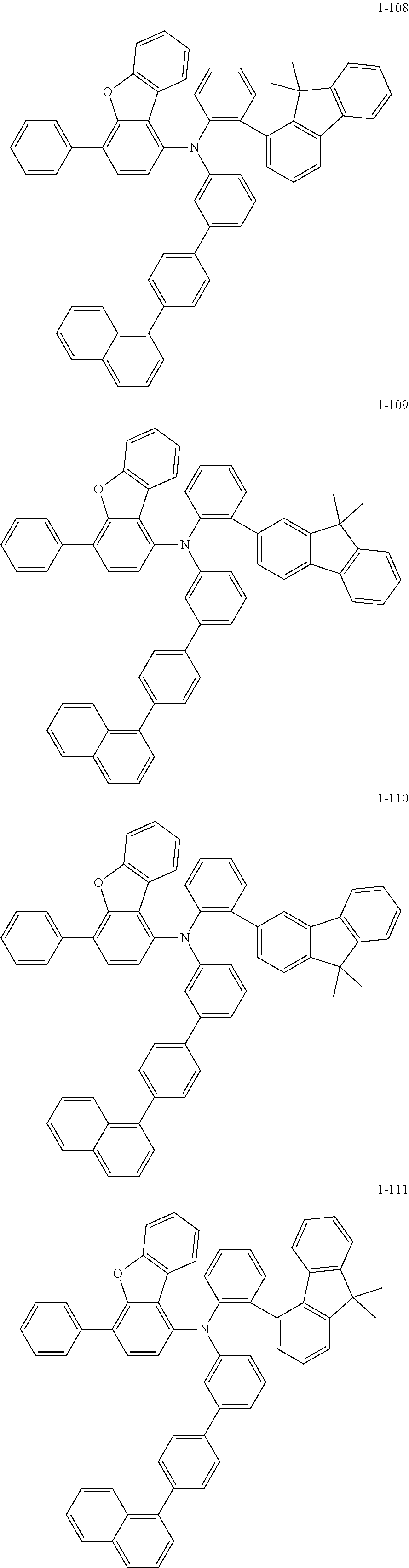

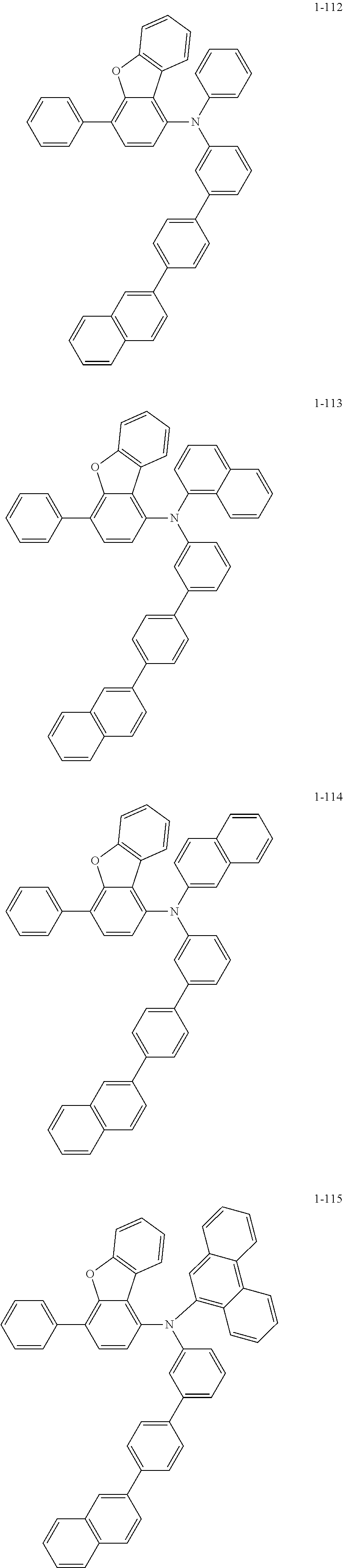 [SMILES: c1ccc(-c2ccc(N(c3ccccc3)c3cccc(-c4ccc(-c5ccc6ccccc6c5)cc4)c3)c3c2oc2ccccc23)cc1 ; c1ccc(-c2ccc(N(c3cccc(-c4ccc(-c5ccc6ccccc6c5)cc4)c3)c3cccc4ccccc34)c3c2oc2ccccc23)cc1 ; c1ccc(-c2ccc(N(c3cccc(-c4ccc(-c5ccc6ccccc6c5)cc4)c3)c3ccc4ccccc4c3)c3c2oc2ccccc23)cc1 ; c1ccc(-c2ccc(N(c3cccc(-c4ccc(-c5ccc6ccccc6c5)cc4)c3)c3cc4ccccc4c4ccccc34)c3c2oc2ccccc23)cc1]

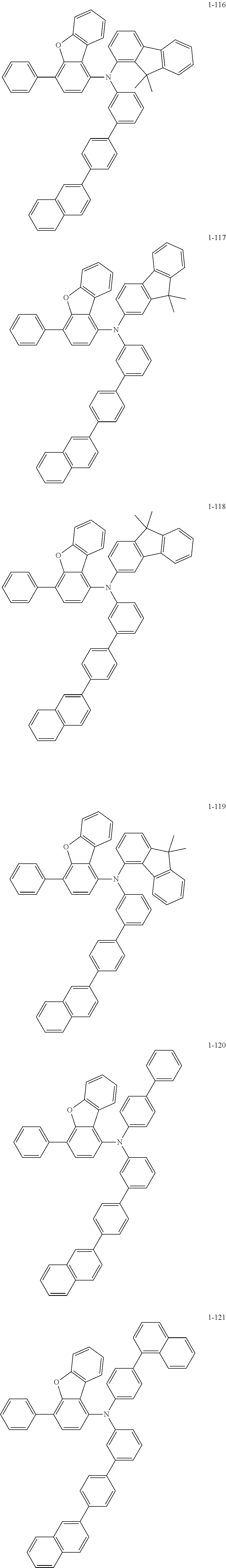

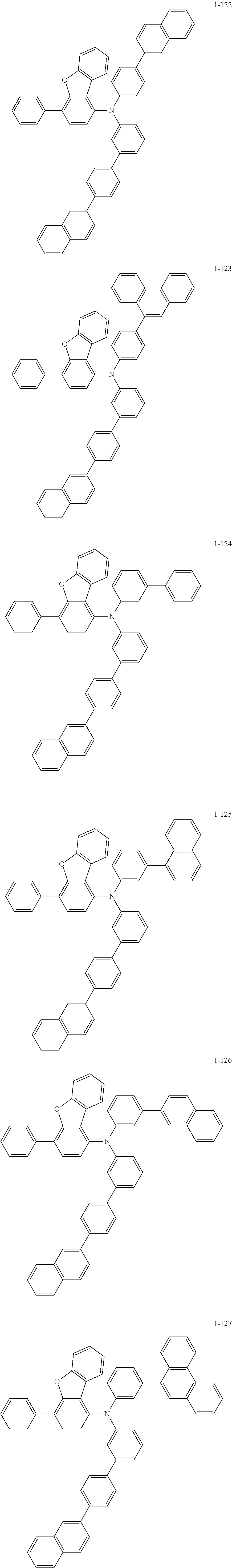

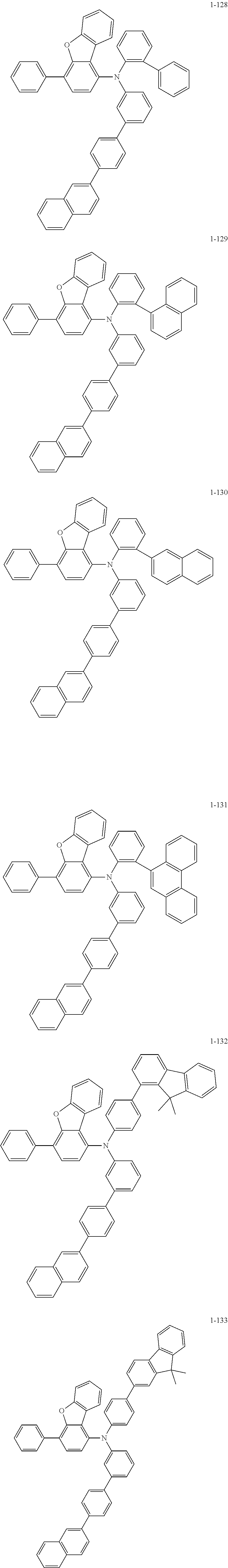

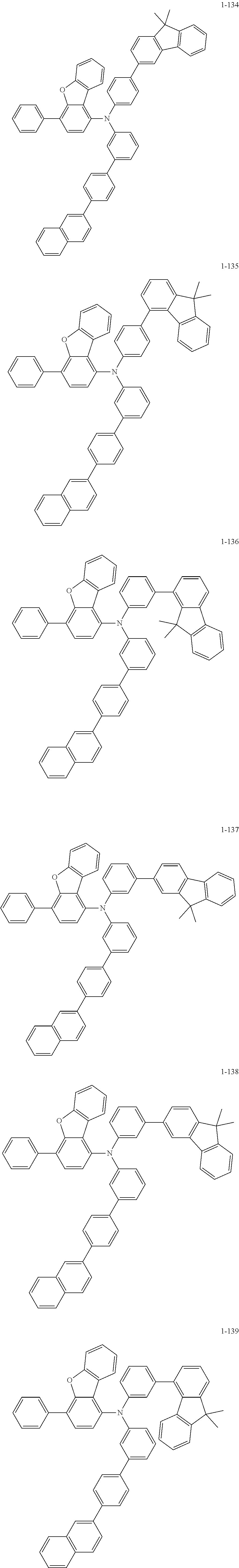

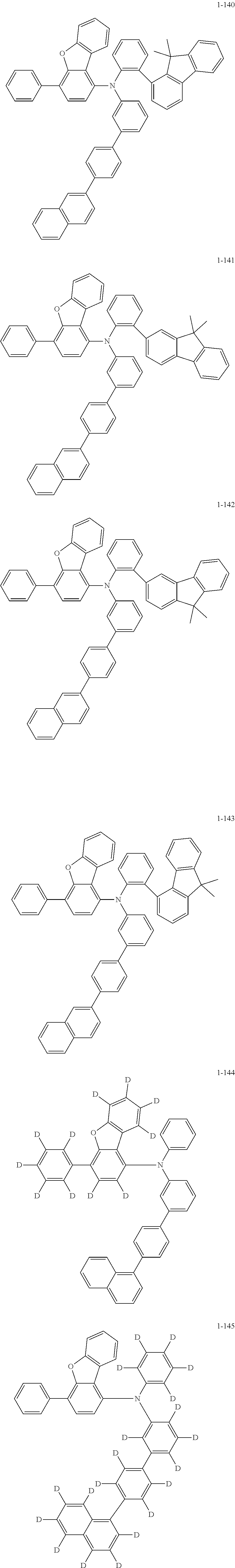

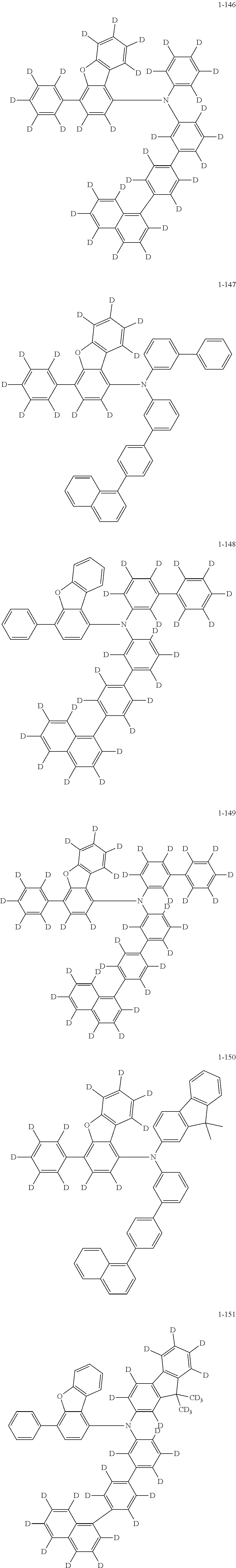

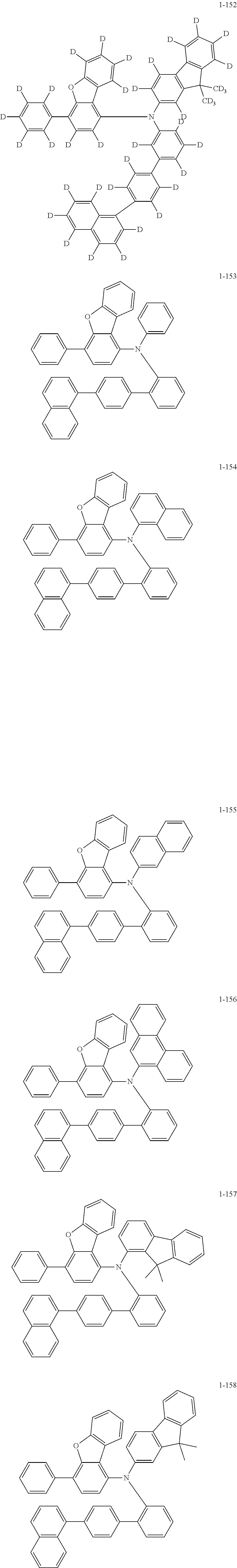

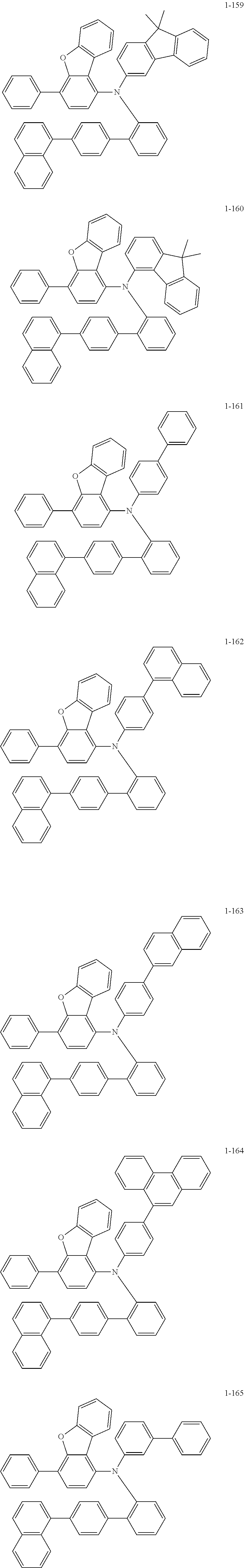

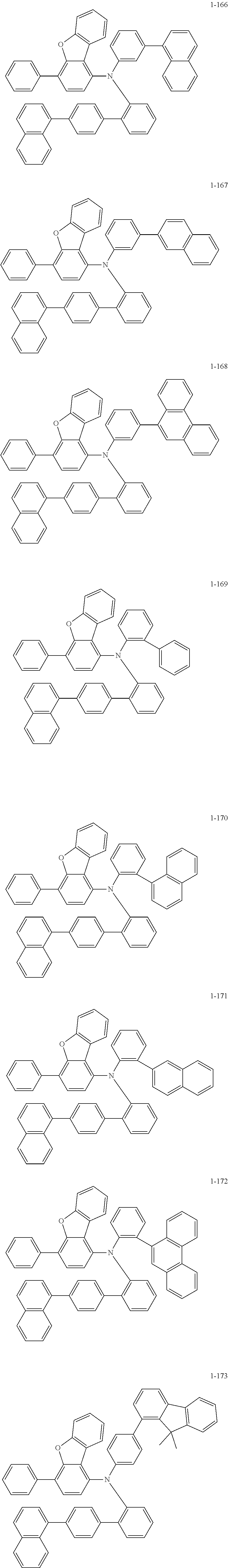

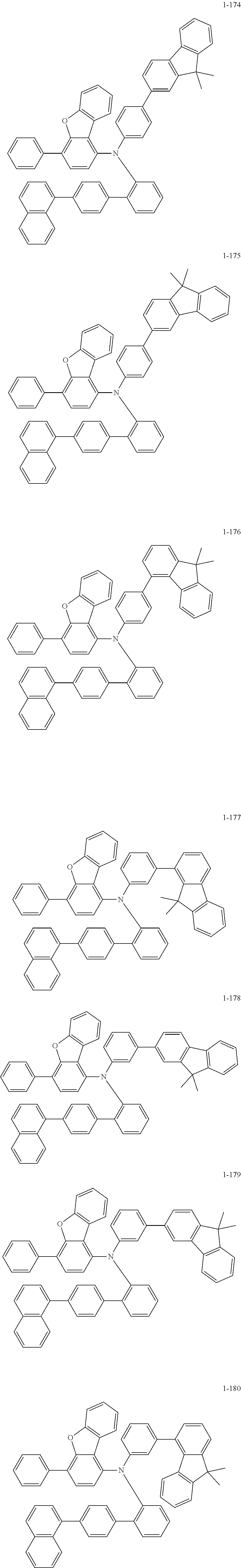

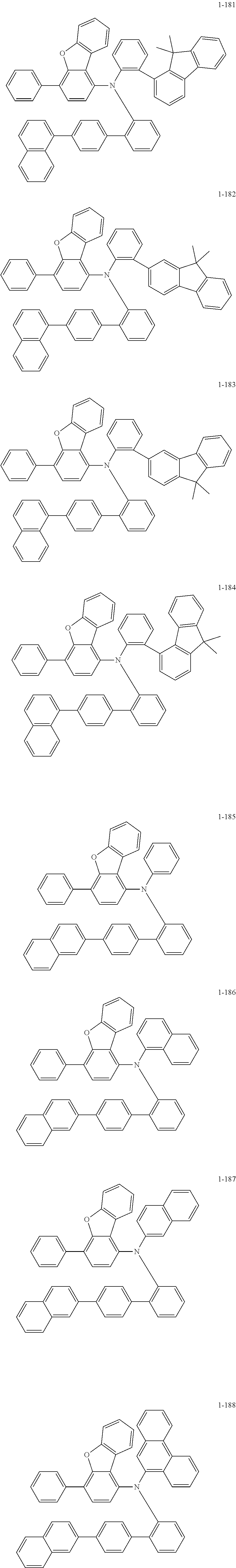

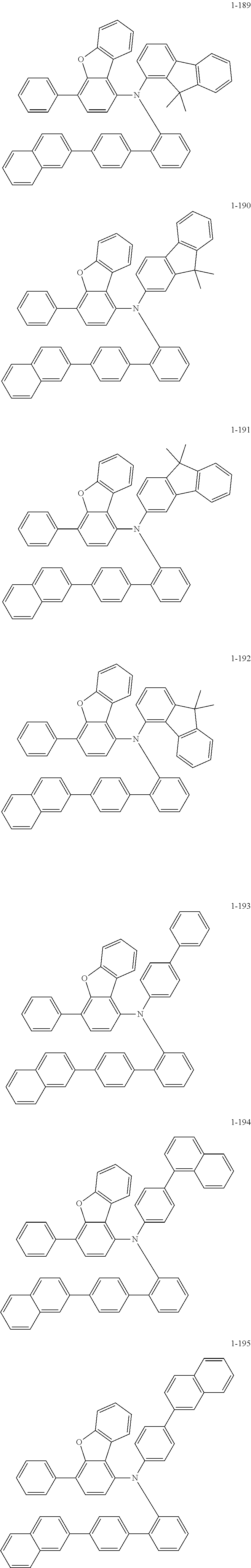

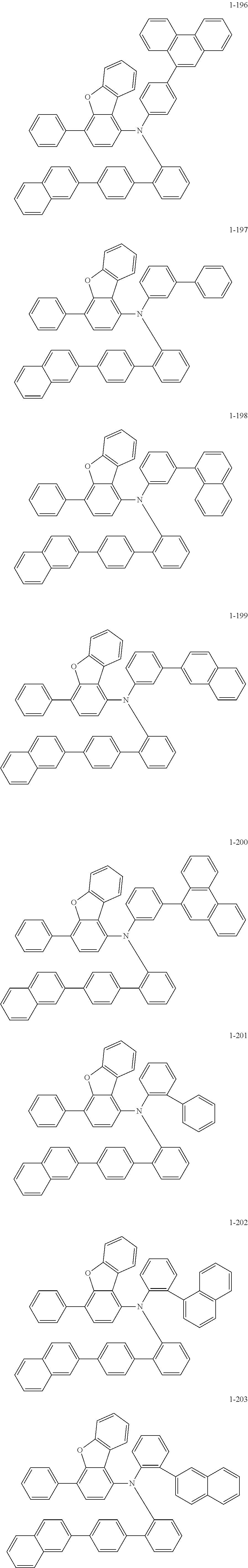

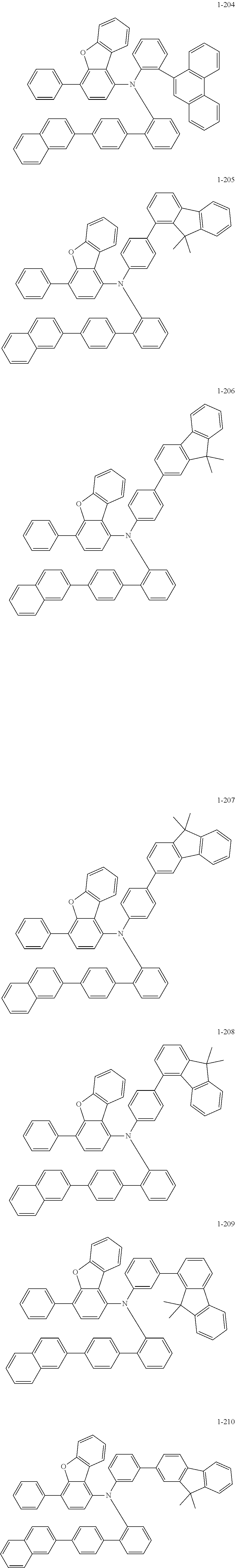

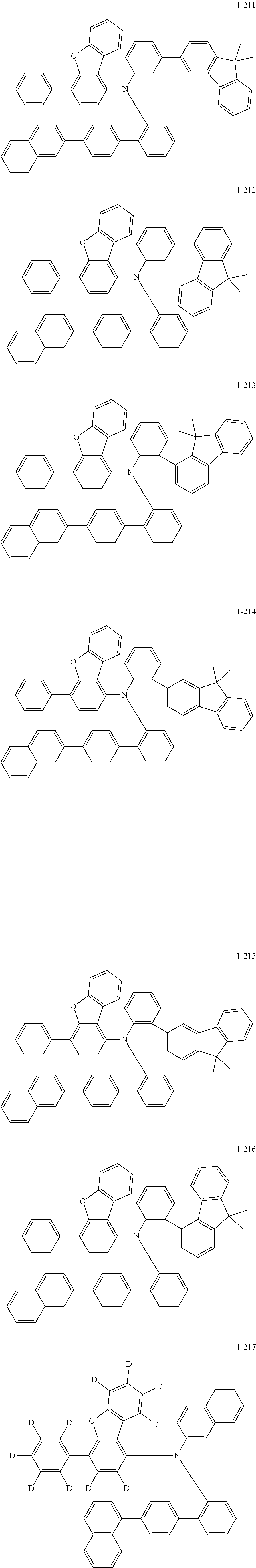

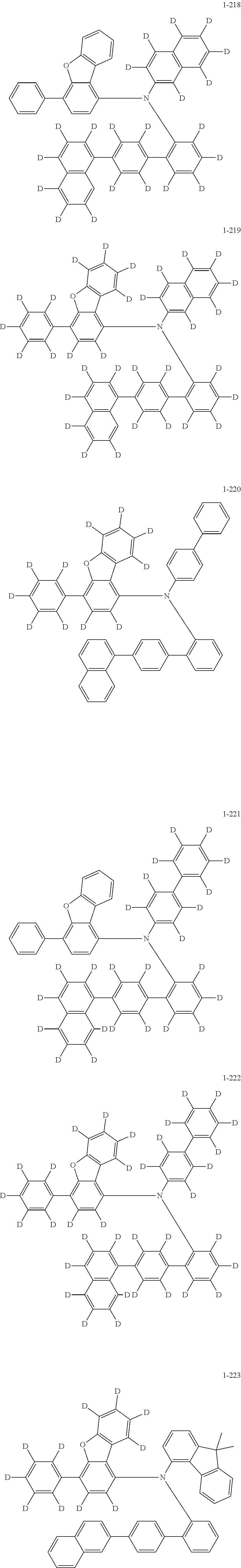

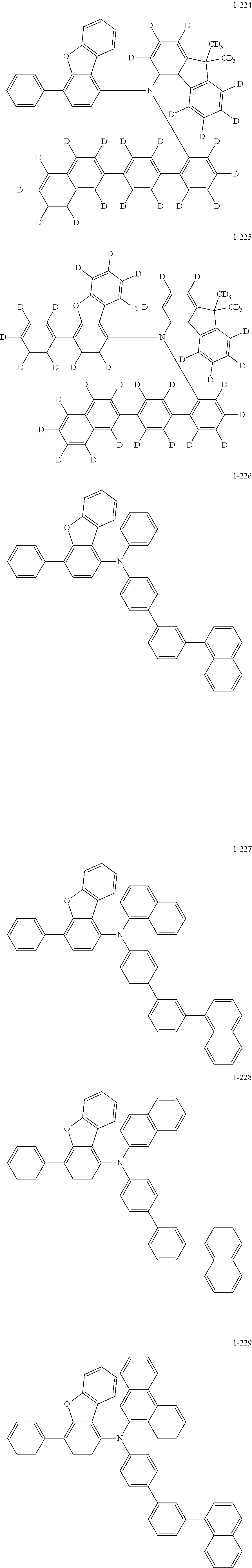

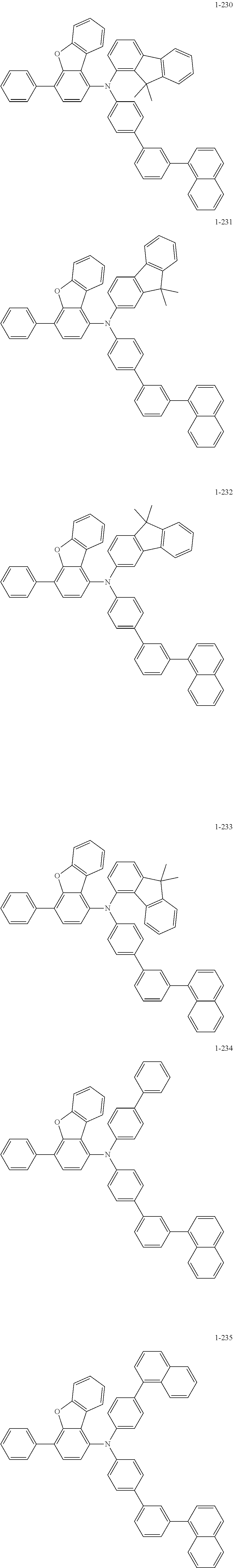

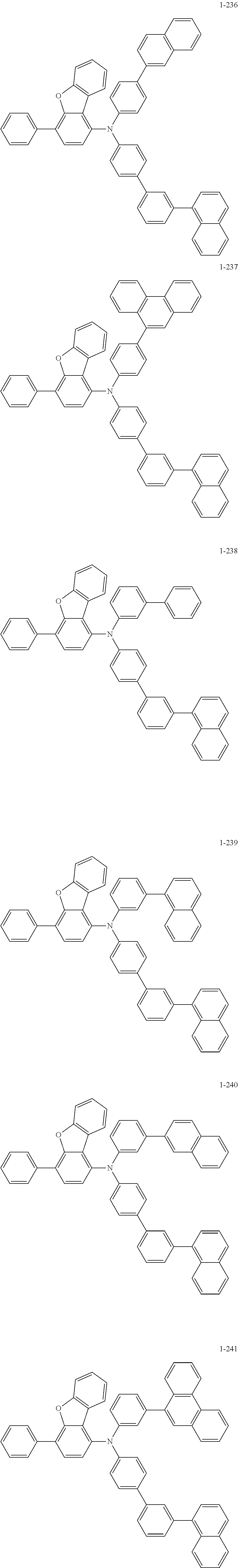

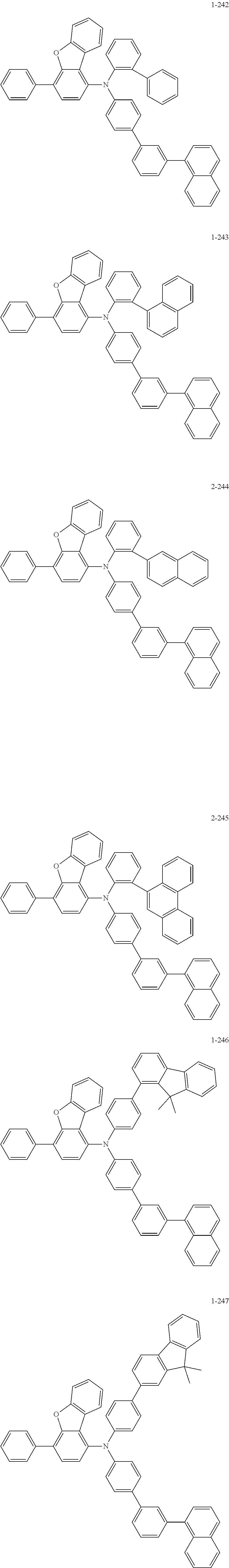

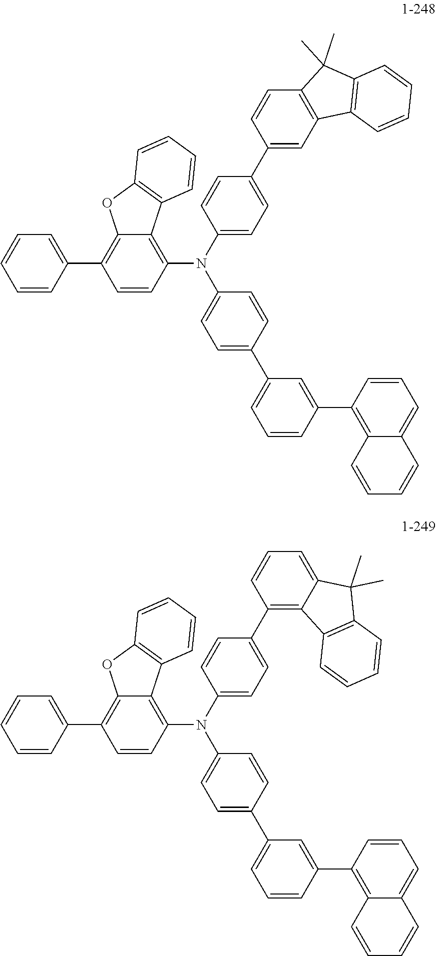 [SMILES: CC1(C)c2ccccc2-c2cc(-c3ccc(N(c4ccc(-c5cccc(-c6cccc7ccccc67)c5)cc4)c4ccc(-c5ccccc5)c5oc6ccccc6c45)cc3)ccc21 ; CC1(C)c2ccccc2-c2c(-c3ccc(N(c4ccc(-c5cccc(-c6cccc7ccccc67)c5)cc4)c4ccc(-c5ccccc5)c5oc6ccccc6c45)cc3)cccc21]

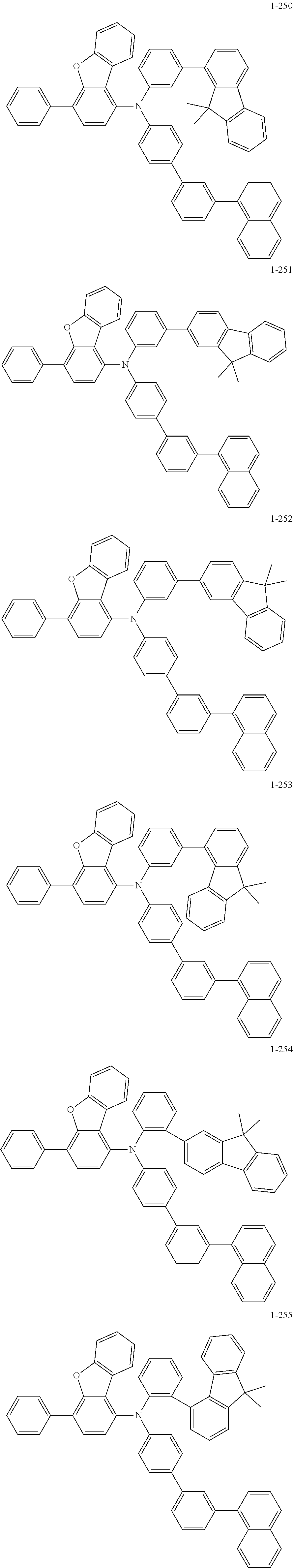

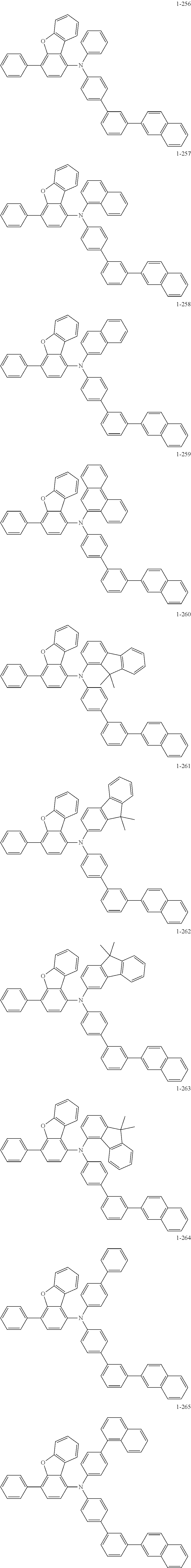

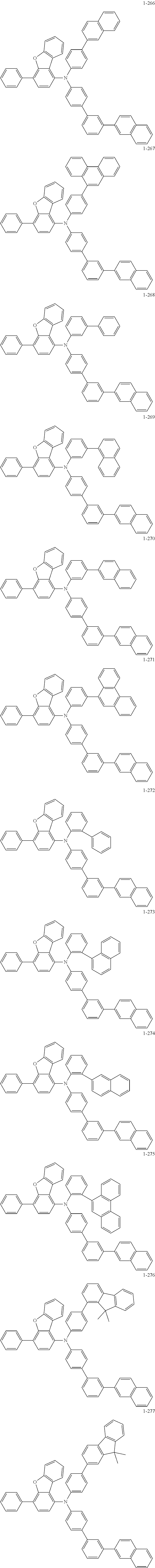

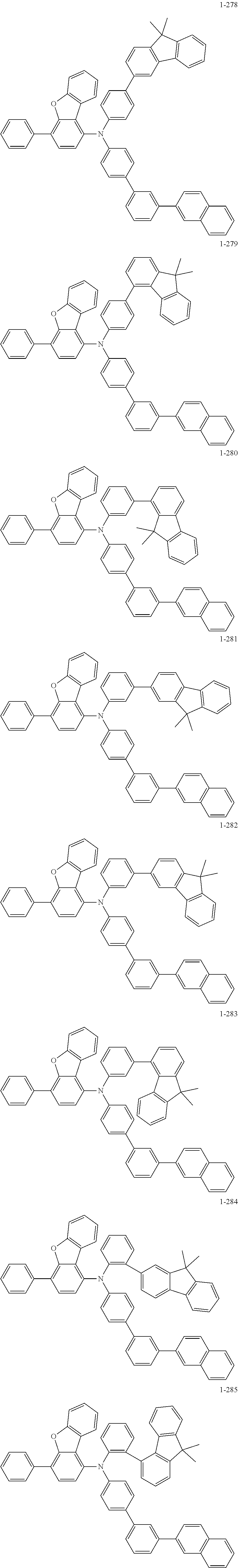

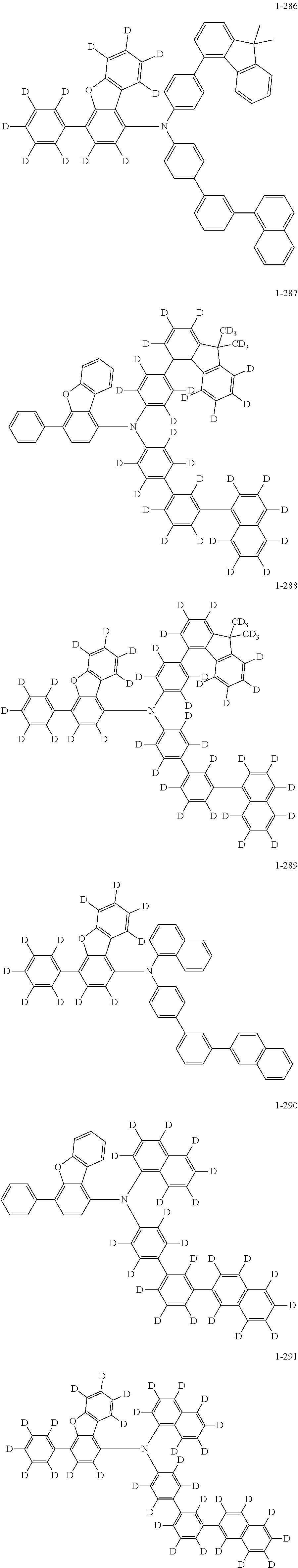

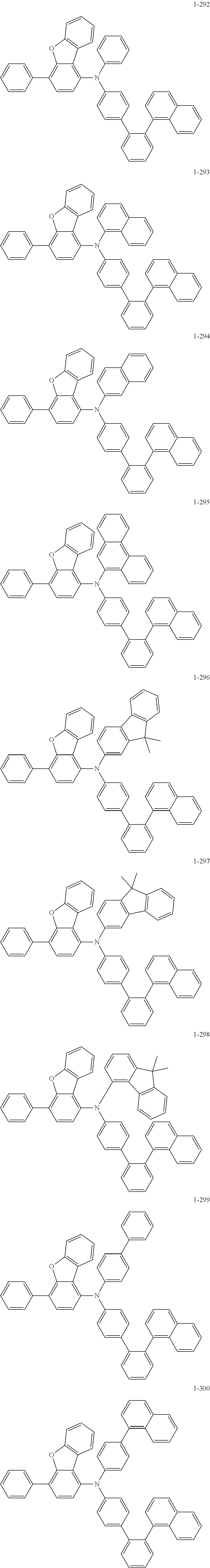

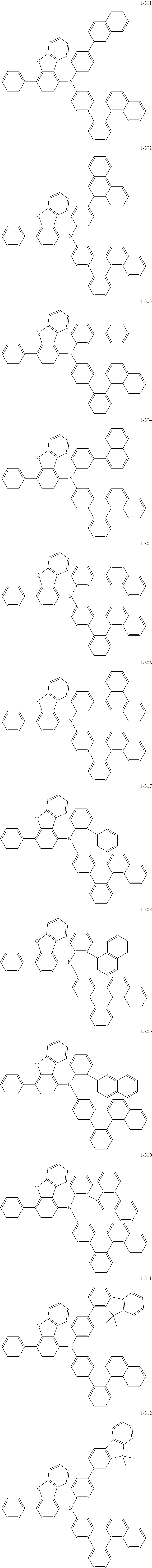

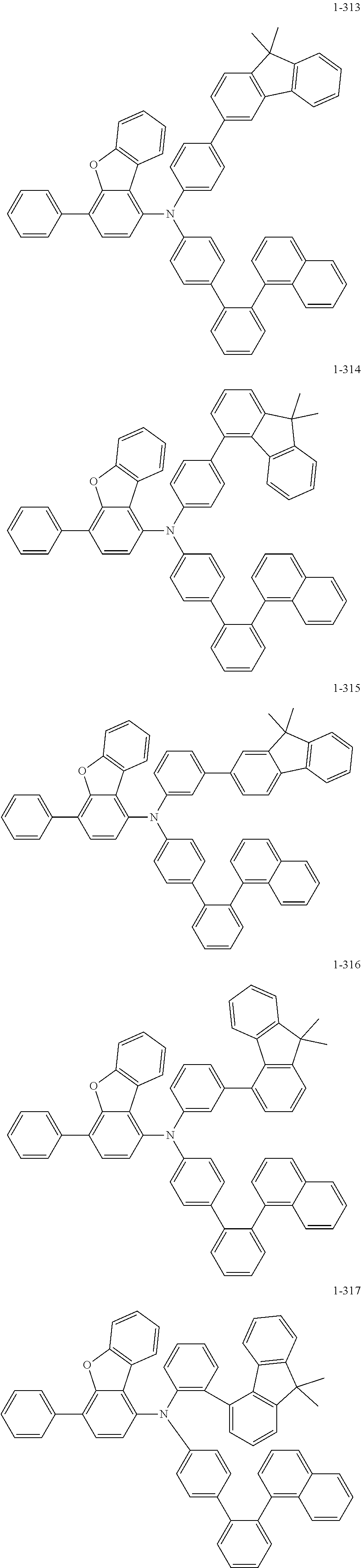

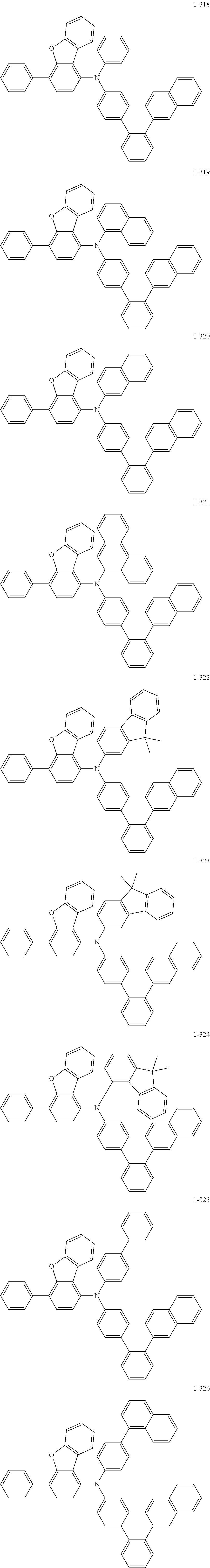

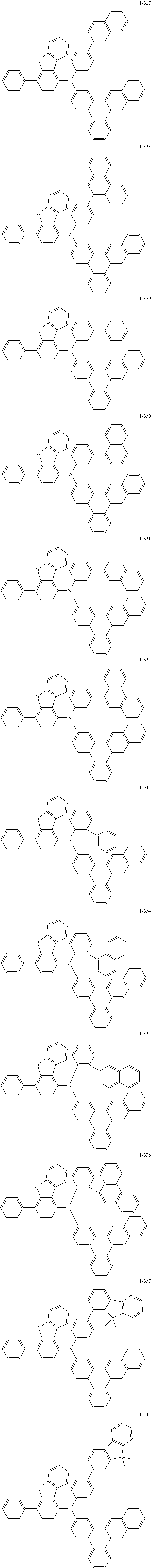

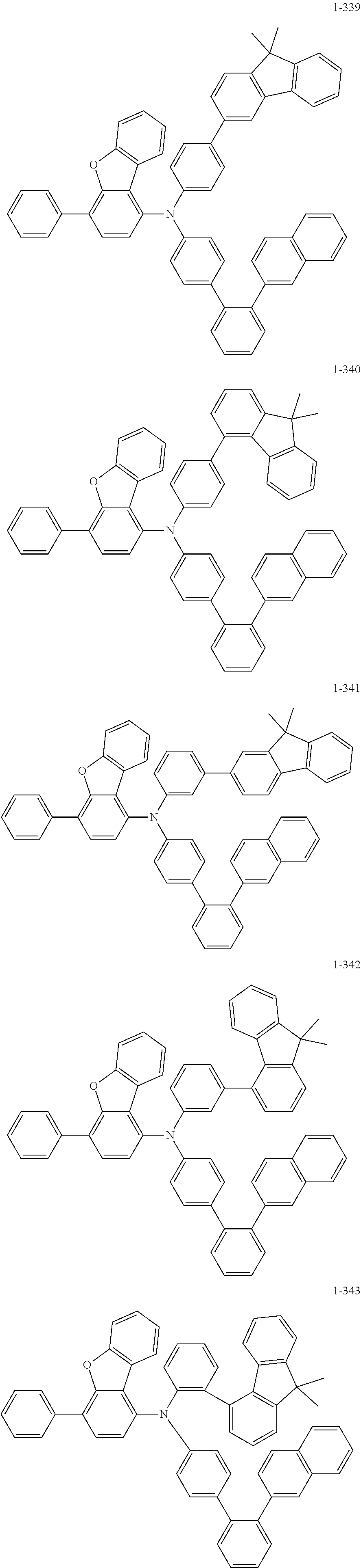

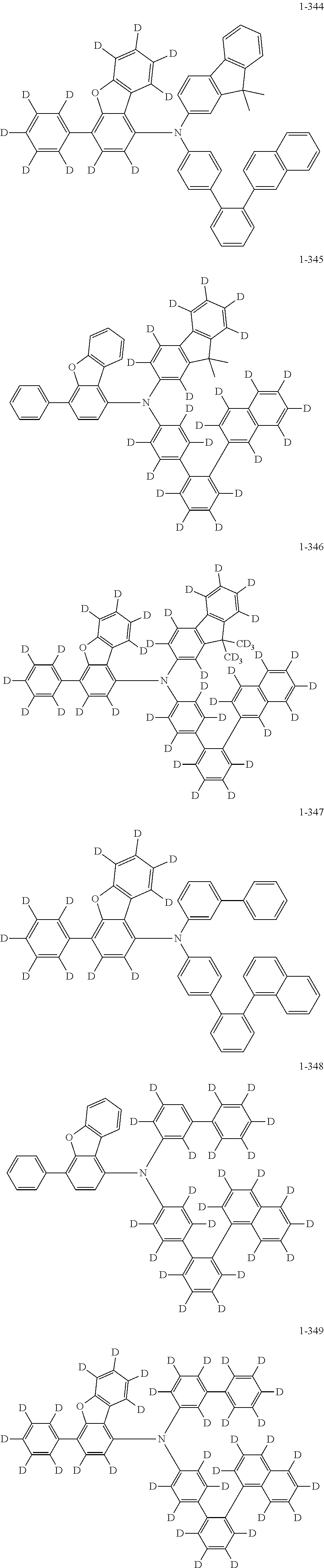

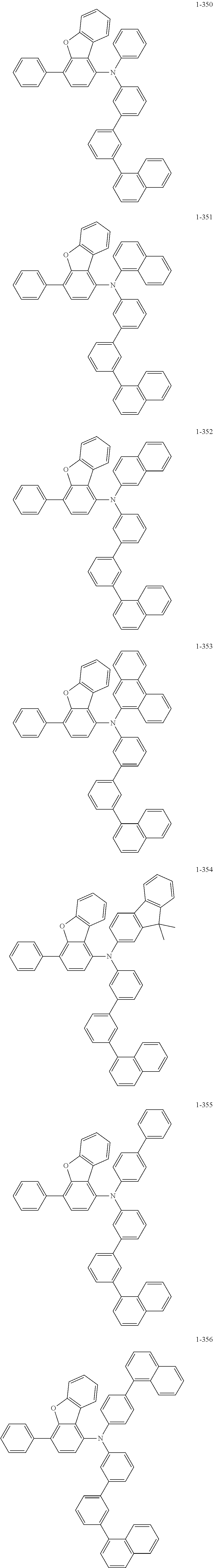

[chemical structure]

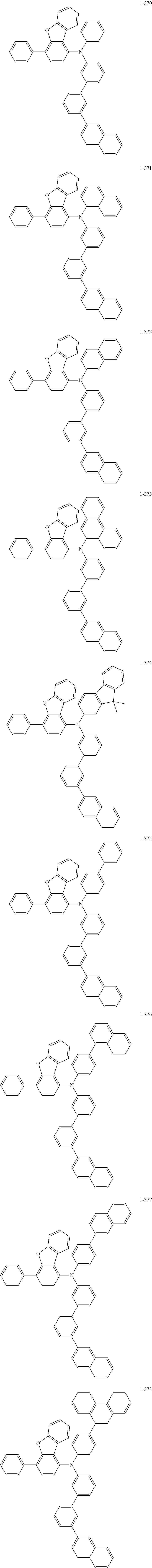

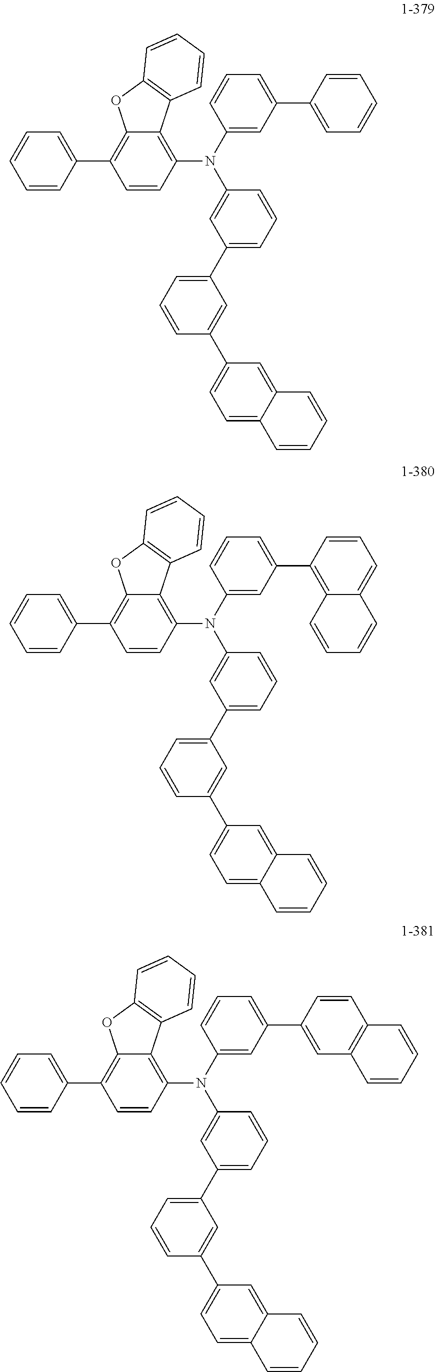 [SMILES: c1ccc(-c2cccc(N(c3cccc(-c4cccc(-c5ccc6ccccc6c5)c4)c3)c3ccc(-c4ccccc4)c4oc5ccccc5c34)c2)cc1 ; c1ccc(-c2ccc(N(c3cccc(-c4cccc(-c5ccc6ccccc6c5)c4)c3)c3cccc(-c4cccc5ccccc45)c3)c3c2oc2ccccc23)cc1 ; c1ccc(-c2ccc(N(c3cccc(-c4cccc(-c5ccc6ccccc6c5)c4)c3)c3cccc(-c4ccc5ccccc5c4)c3)c3c2oc2ccccc23)cc1]

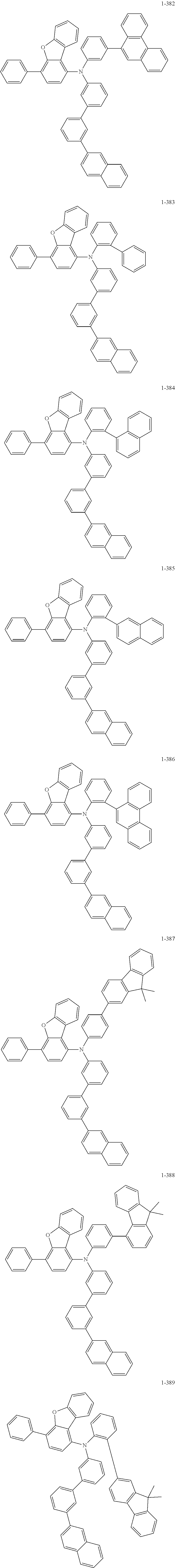

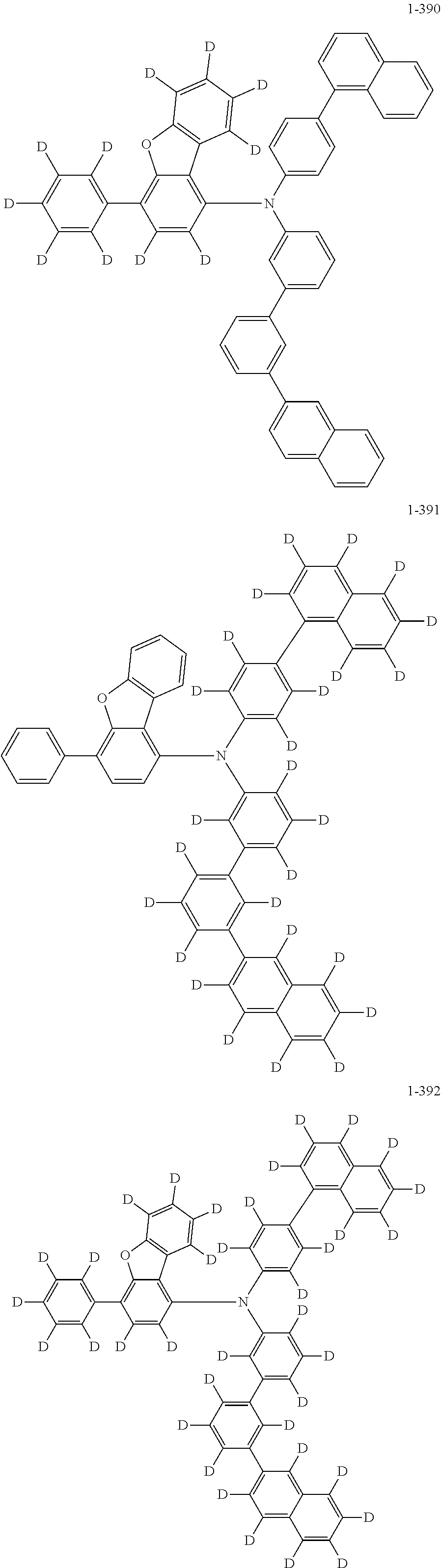

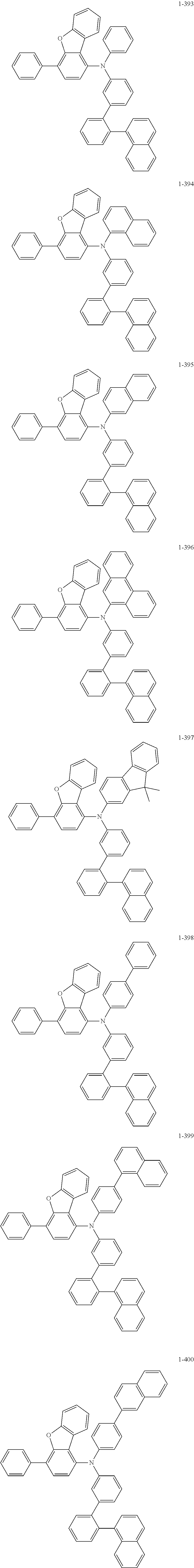

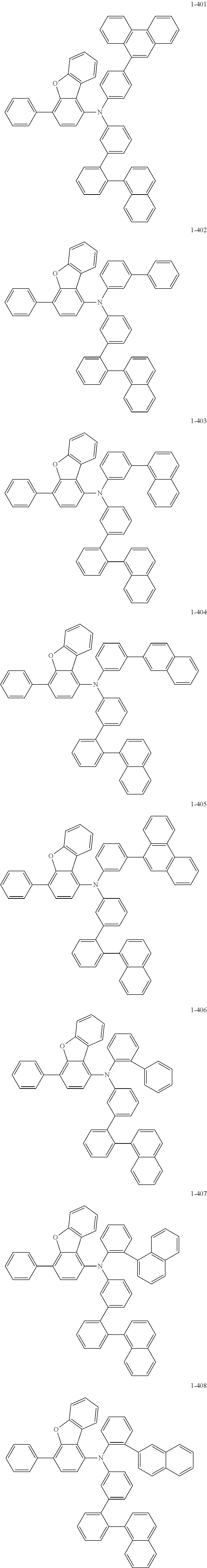

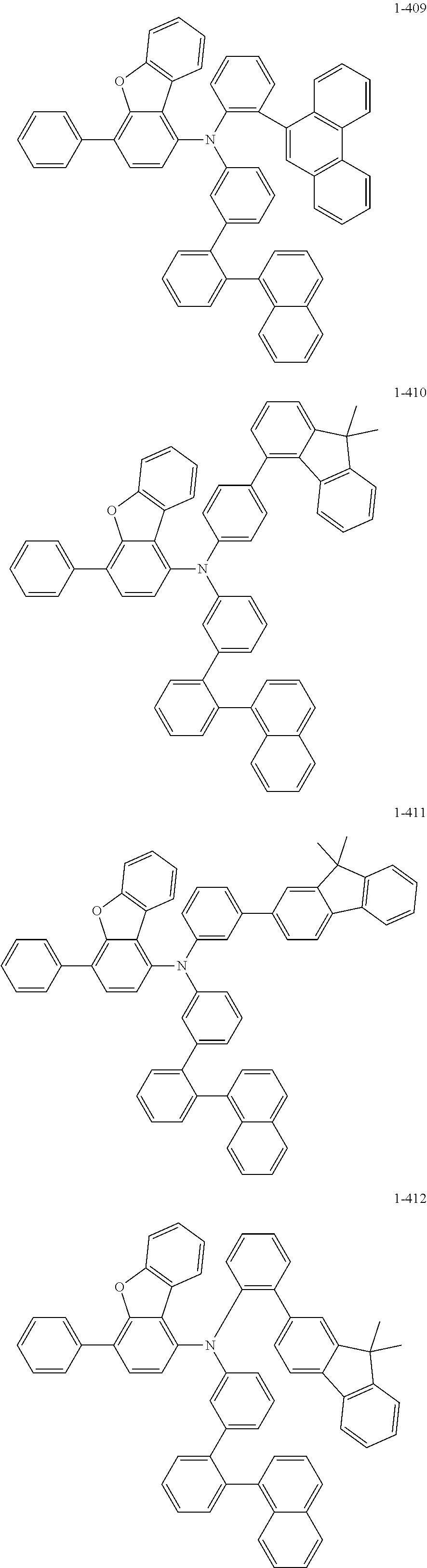

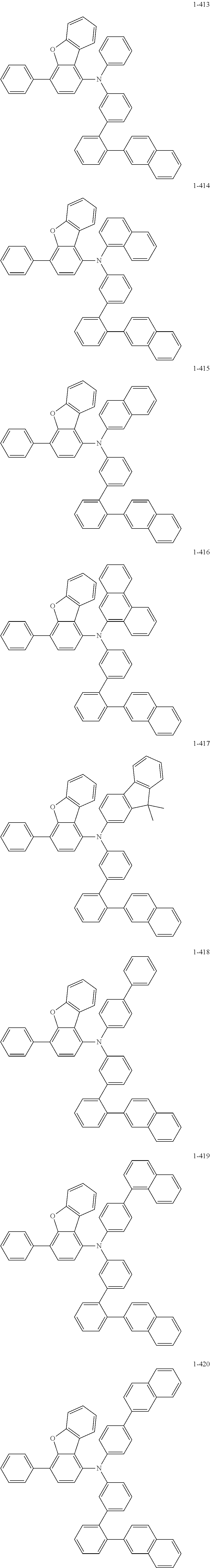

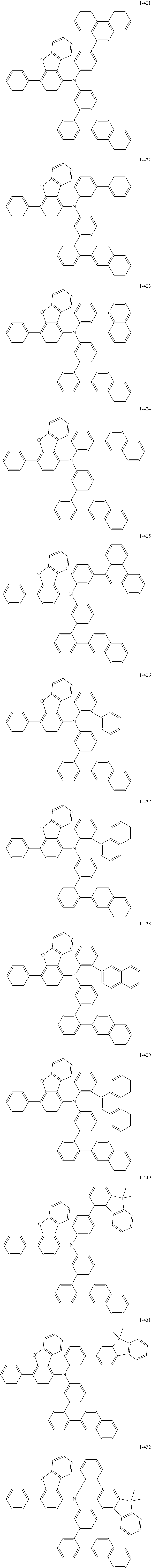

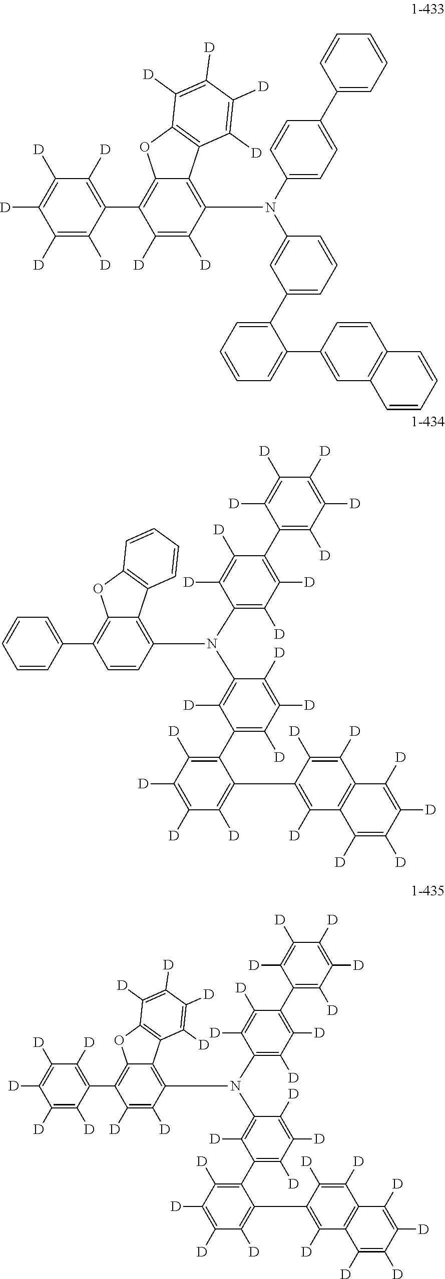

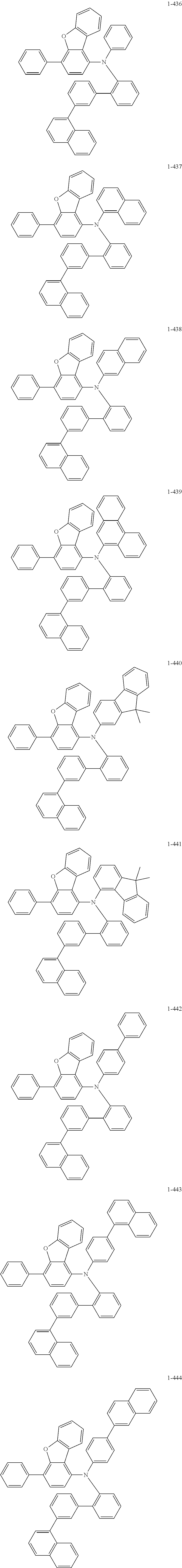

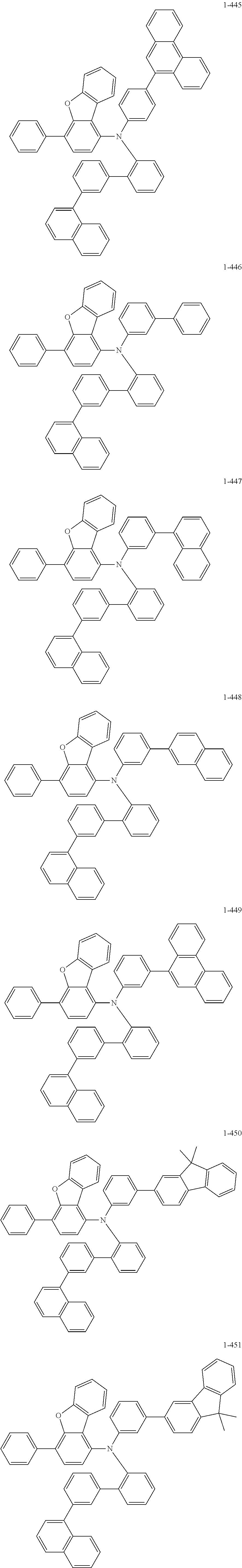

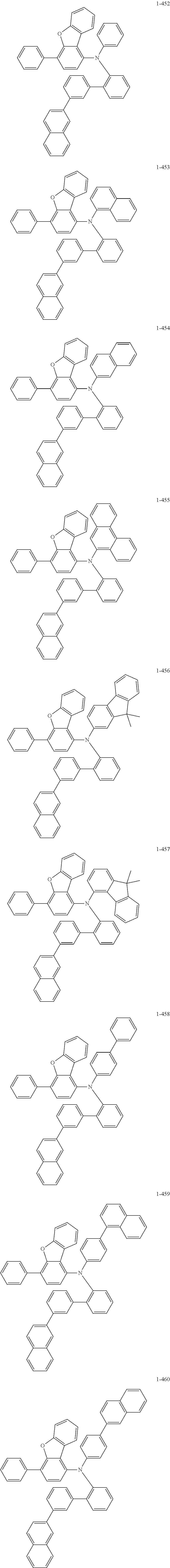

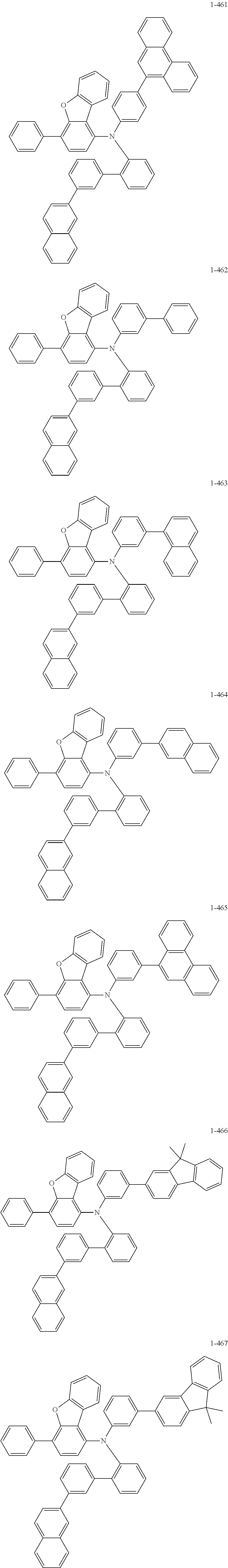

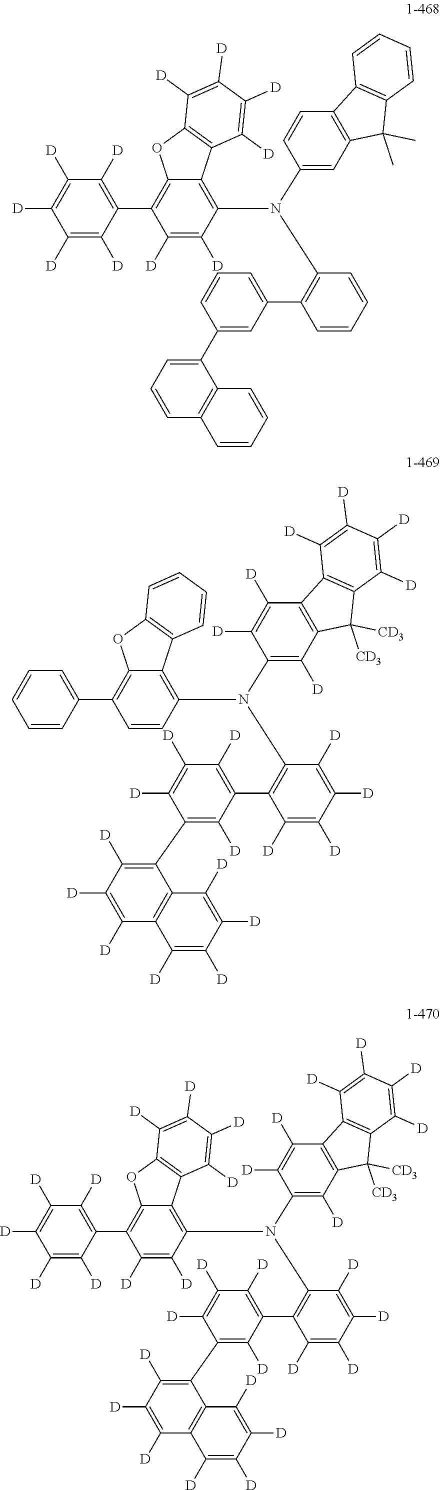

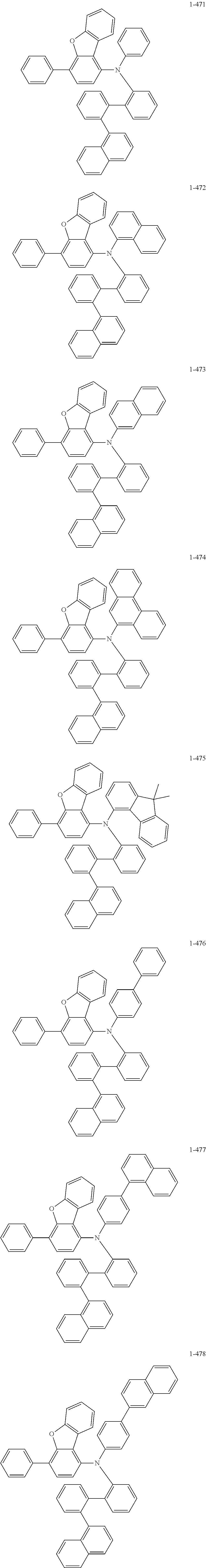

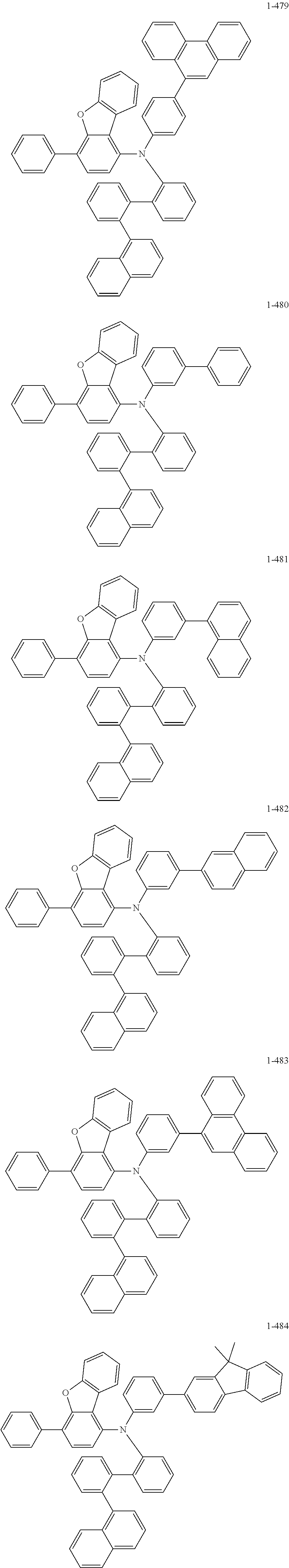

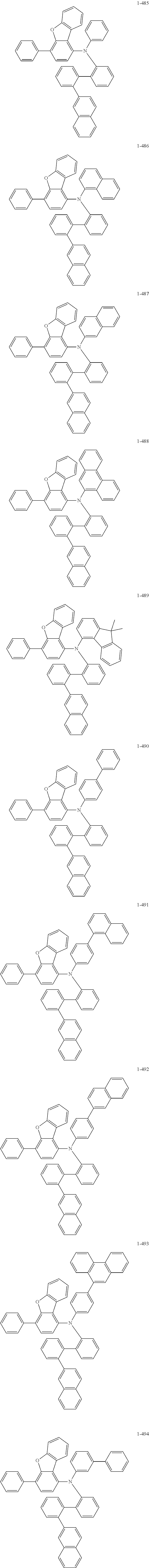

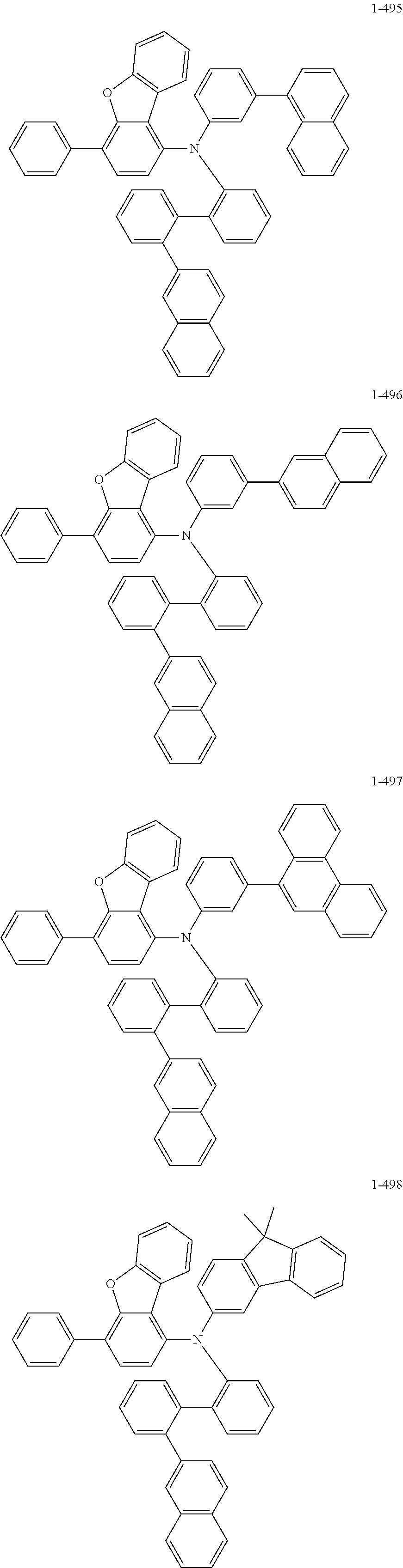 [SMILES: c1ccc(-c2ccc(N(c3cccc(-c4cccc5ccccc45)c3)c3ccccc3-c3ccccc3-c3ccc4ccccc4c3)c3c2oc2ccccc23)cc1 ; c1ccc(-c2ccc(N(c3cccc(-c4ccc5ccccc5c4)c3)c3ccccc3-c3ccccc3-c3ccc4ccccc4c3)c3c2oc2ccccc23)cc1 ; c1ccc(-c2ccc(N(c3cccc(-c4cc5ccccc5c5ccccc45)c3)c3ccccc3-c3ccccc3-c3ccc4ccccc4c3)c3c2oc2ccccc23)cc1 ; CC1(C)c2ccccc2-c2cc(N(c3ccccc3-c3ccccc3-c3ccc4ccccc4c3)c3ccc(-c4ccccc4)c4oc5ccccc5c34)ccc21]

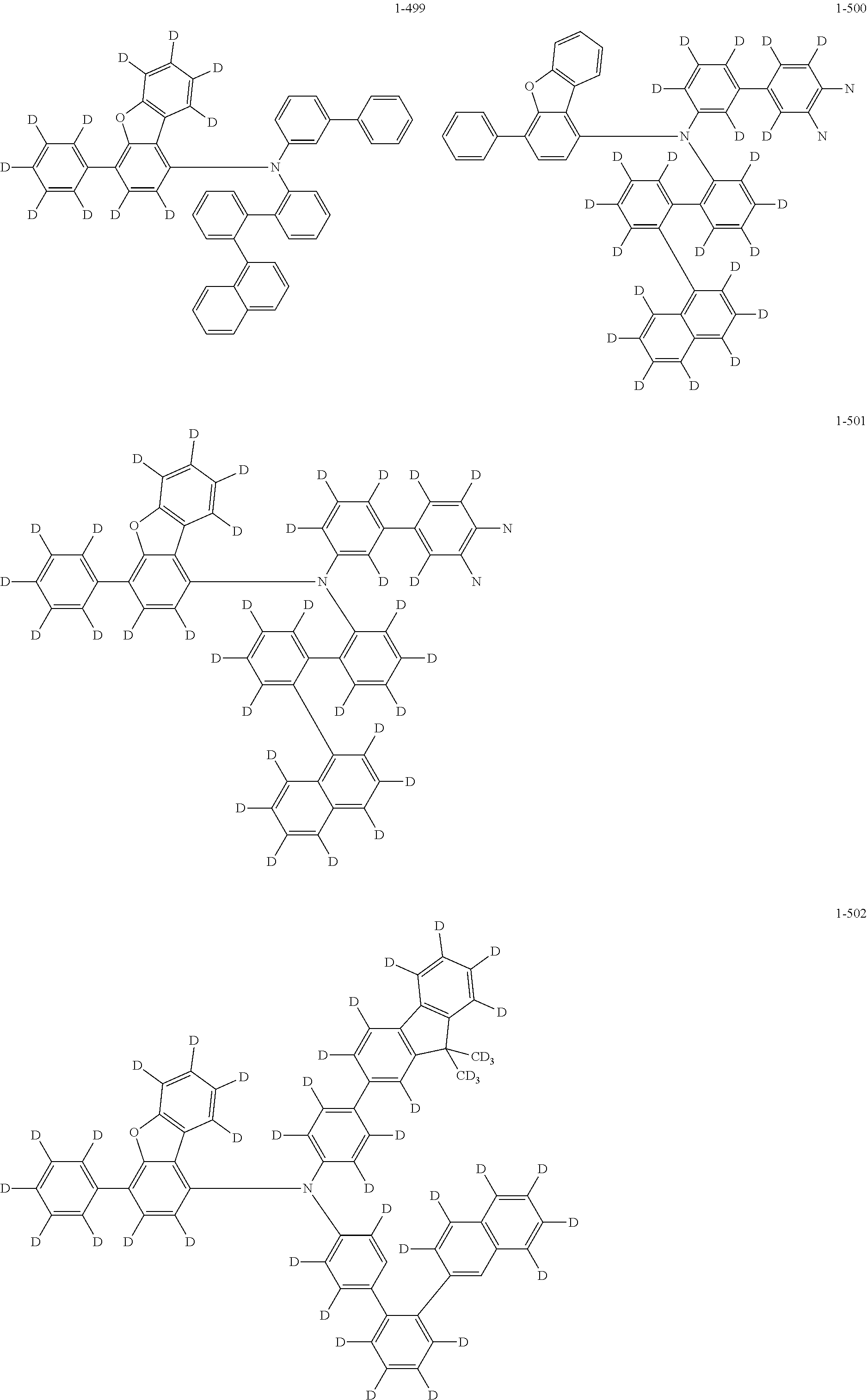

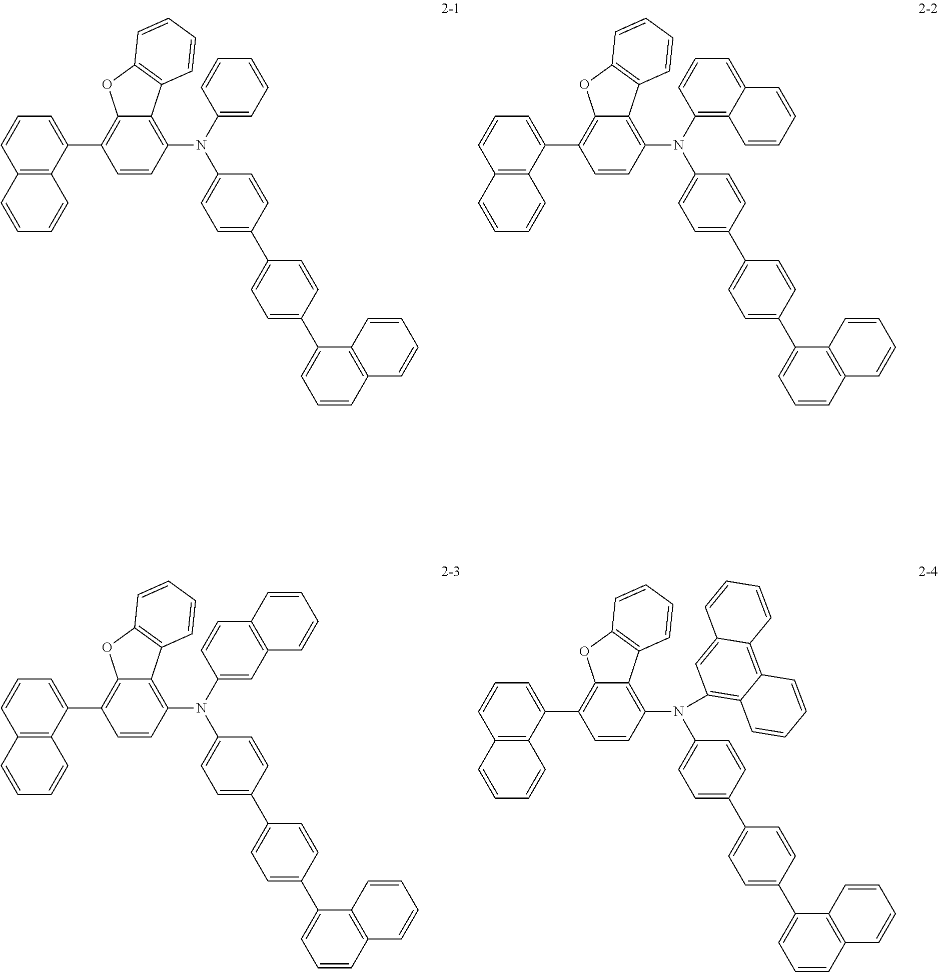 [SMILES: c1ccc(N(c2ccc(-c3ccc(-c4cccc5ccccc45)cc3)cc2)c2ccc(-c3cccc4ccccc34)c3oc4ccccc4c23)cc1 ; c1ccc2c(-c3ccc(-c4ccc(N(c5cccc6ccccc56)c5ccc(-c6cccc7ccccc67)c6oc7ccccc7c56)cc4)cc3)cccc2c1 ; c1ccc2cc(N(c3ccc(-c4ccc(-c5cccc6ccccc56)cc4)cc3)c3ccc(-c4cccc5ccccc45)c4oc5ccccc5c34)ccc2c1 ; c1ccc2c(-c3ccc(-c4ccc(N(c5cc6ccccc6c6ccccc56)c5ccc(-c6cccc7ccccc67)c6oc7ccccc7c56)cc4)cc3)cccc2c1]

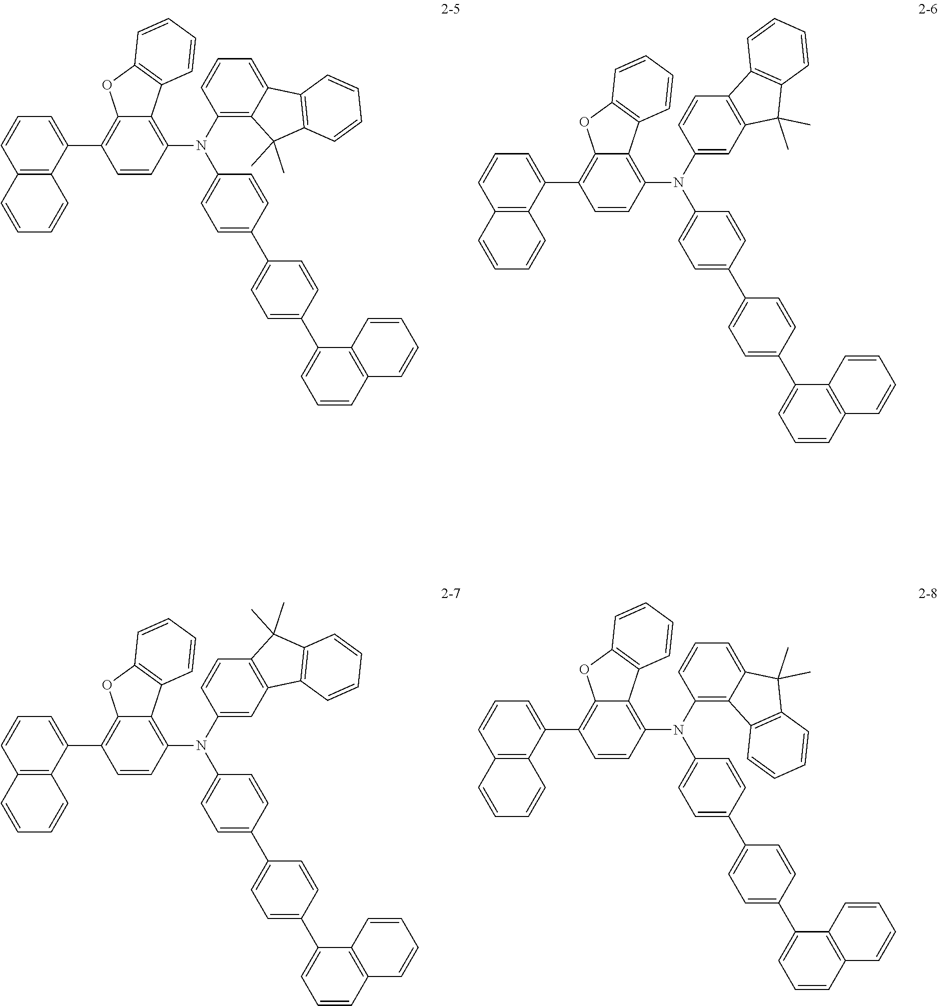 [SMILES: CC1(C)c2ccccc2-c2cccc(N(c3ccc(-c4ccc(-c5cccc6ccccc56)cc4)cc3)c3ccc(-c4cccc5ccccc45)c4oc5ccccc5c34)c21 ; CC1(C)c2ccccc2-c2ccc(N(c3ccc(-c4ccc(-c5cccc6ccccc56)cc4)cc3)c3ccc(-c4cccc5ccccc45)c4oc5ccccc5c34)cc21 ; CC1(C)c2ccccc2-c2cc(N(c3ccc(-c4ccc(-c5cccc6ccccc56)cc4)cc3)c3ccc(-c4cccc5ccccc45)c4oc5ccccc5c34)ccc21 ; CC1(C)c2ccccc2-c2c(N(c3ccc(-c4ccc(-c5cccc6ccccc56)cc4)cc3)c3ccc(-c4cccc5ccccc45)c4oc5ccccc5c34)cccc21]

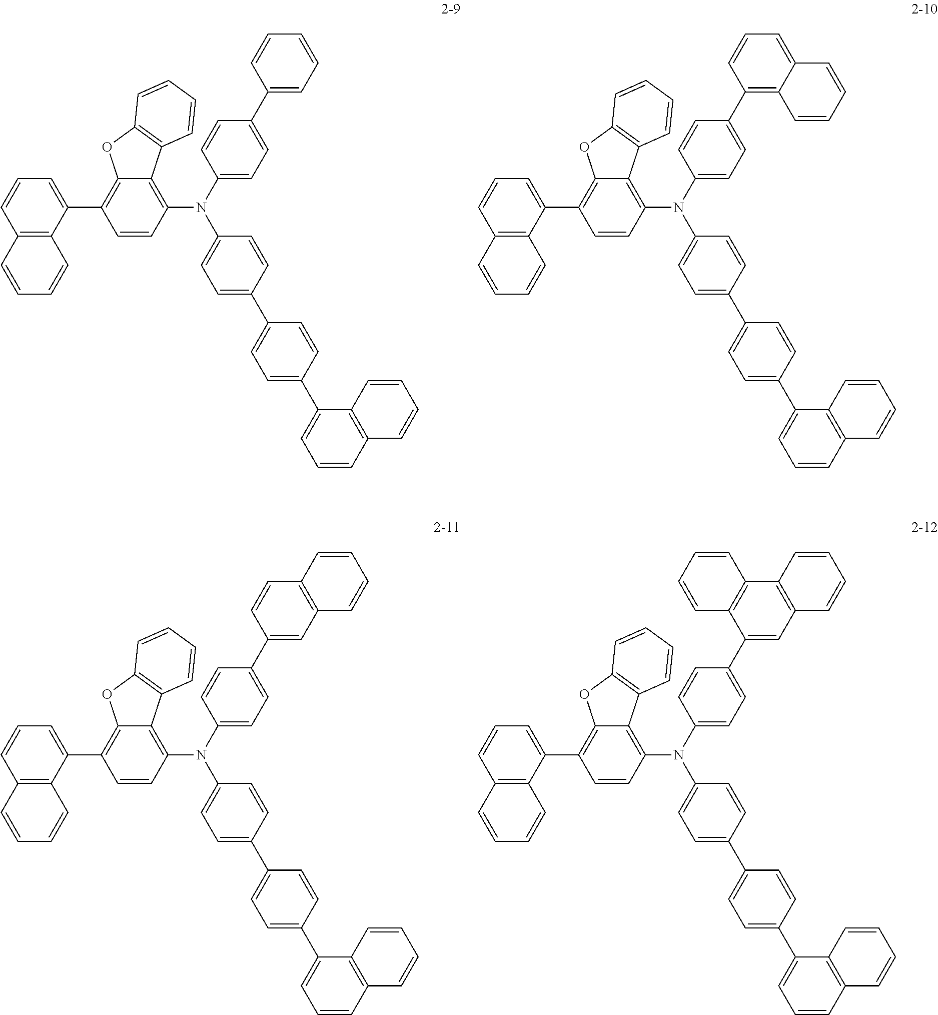 [SMILES: c1ccc(-c2ccc(N(c3ccc(-c4ccc(-c5cccc6ccccc56)cc4)cc3)c3ccc(-c4cccc5ccccc45)c4oc5ccccc5c34)cc2)cc1 ; c1ccc2c(-c3ccc(-c4ccc(N(c5ccc(-c6cccc7ccccc67)cc5)c5ccc(-c6cccc7ccccc67)c6oc7ccccc7c56)cc4)cc3)cccc2c1 ; c1ccc2cc(-c3ccc(N(c4ccc(-c5ccc(-c6cccc7ccccc67)cc5)cc4)c4ccc(-c5cccc6ccccc56)c5oc6ccccc6c45)cc3)ccc2c1 ; c1ccc2c(-c3ccc(-c4ccc(N(c5ccc(-c6cc7ccccc7c7ccccc67)cc5)c5ccc(-c6cccc7ccccc67)c6oc7ccccc7c56)cc4)cc3)cccc2c1]

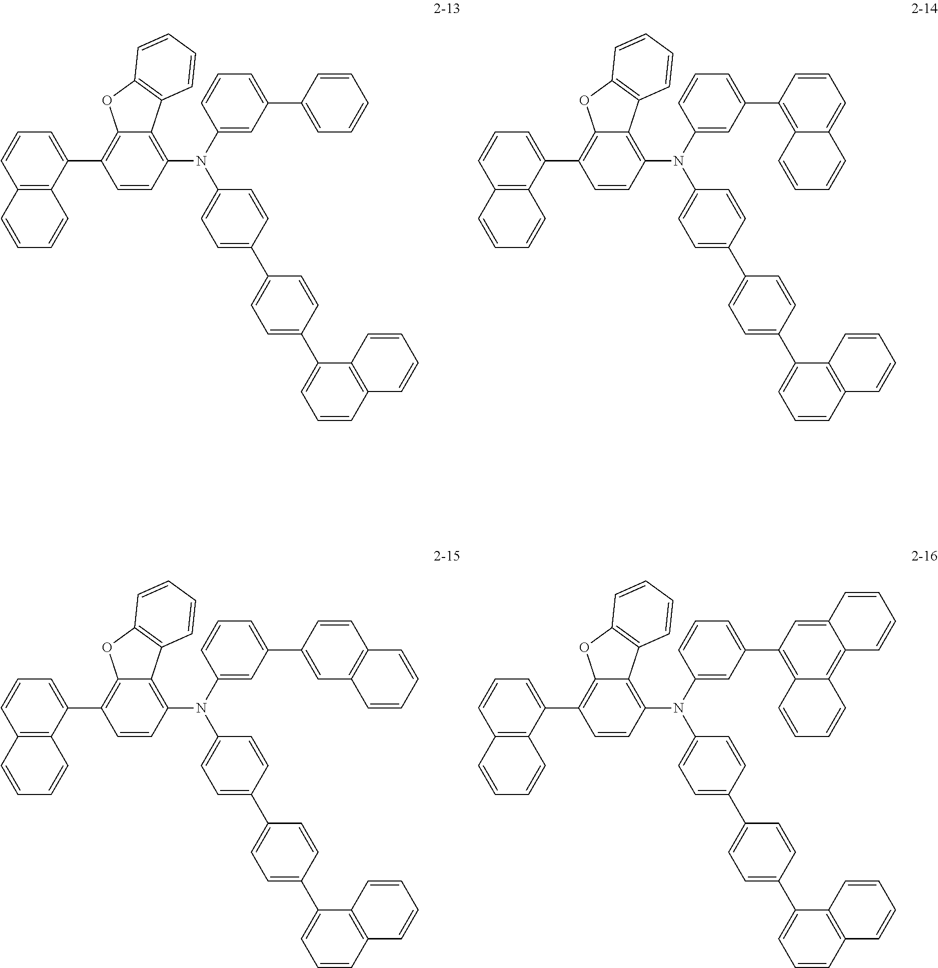 [SMILES: c1ccc(-c2cccc(N(c3ccc(-c4ccc(-c5cccc6ccccc56)cc4)cc3)c3ccc(-c4cccc5ccccc45)c4oc5ccccc5c34)c2)cc1 ; c1cc(-c2cccc3ccccc23)cc(N(c2ccc(-c3ccc(-c4cccc5ccccc45)cc3)cc2)c2ccc(-c3cccc4ccccc34)c3oc4ccccc4c23)c1 ; c1cc(-c2ccc3ccccc3c2)cc(N(c2ccc(-c3ccc(-c4cccc5ccccc45)cc3)cc2)c2ccc(-c3cccc4ccccc34)c3oc4ccccc4c23)c1 ; c1cc(-c2cc3ccccc3c3ccccc23)cc(N(c2ccc(-c3ccc(-c4cccc5ccccc45)cc3)cc2)c2ccc(-c3cccc4ccccc34)c3oc4ccccc4c23)c1]

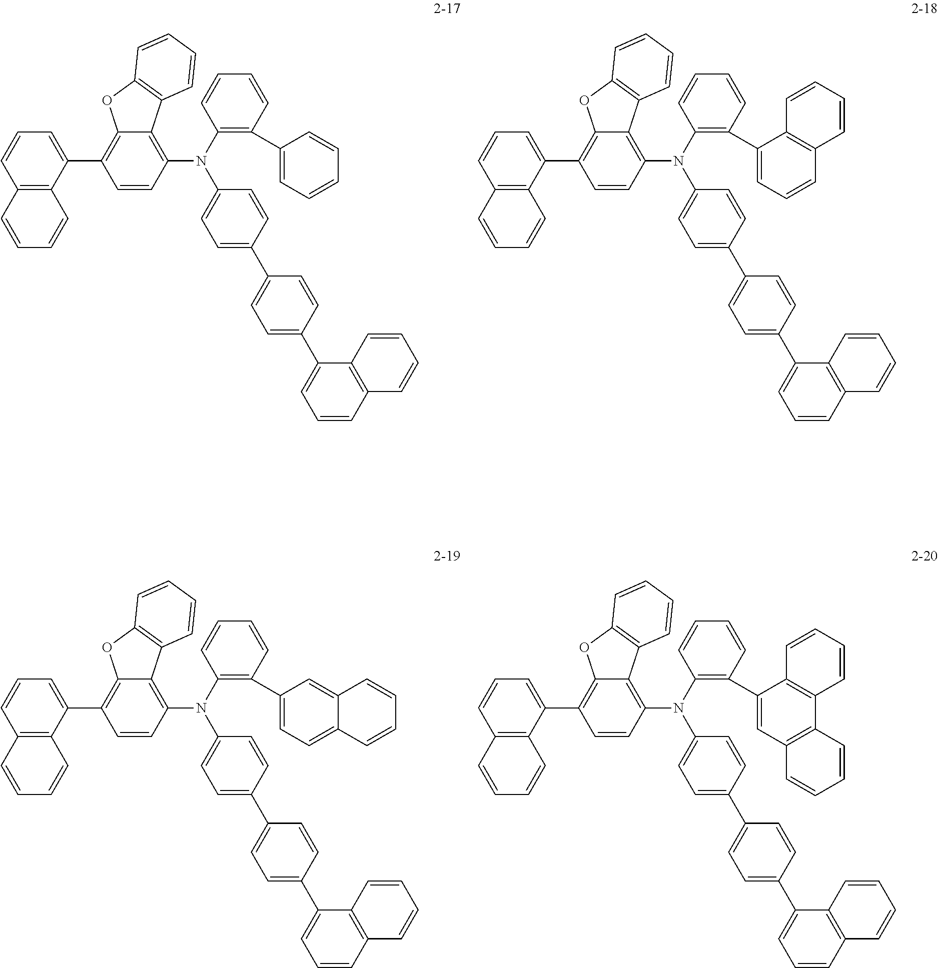 [SMILES: c1ccc(-c2ccccc2N(c2ccc(-c3ccc(-c4cccc5ccccc45)cc3)cc2)c2ccc(-c3cccc4ccccc34)c3oc4ccccc4c23)cc1 ; c1ccc(N(c2ccc(-c3ccc(-c4cccc5ccccc45)cc3)cc2)c2ccc(-c3cccc4ccccc34)c3oc4ccccc4c23)c(-c2cccc3ccccc23)c1 ; c1ccc(N(c2ccc(-c3ccc(-c4cccc5ccccc45)cc3)cc2)c2ccc(-c3cccc4ccccc34)c3oc4ccccc4c23)c(-c2ccc3ccccc3c2)c1 ; c1ccc(N(c2ccc(-c3ccc(-c4cccc5ccccc45)cc3)cc2)c2ccc(-c3cccc4ccccc34)c3oc4ccccc4c23)c(-c2cc3ccccc3c3ccccc23)c1]

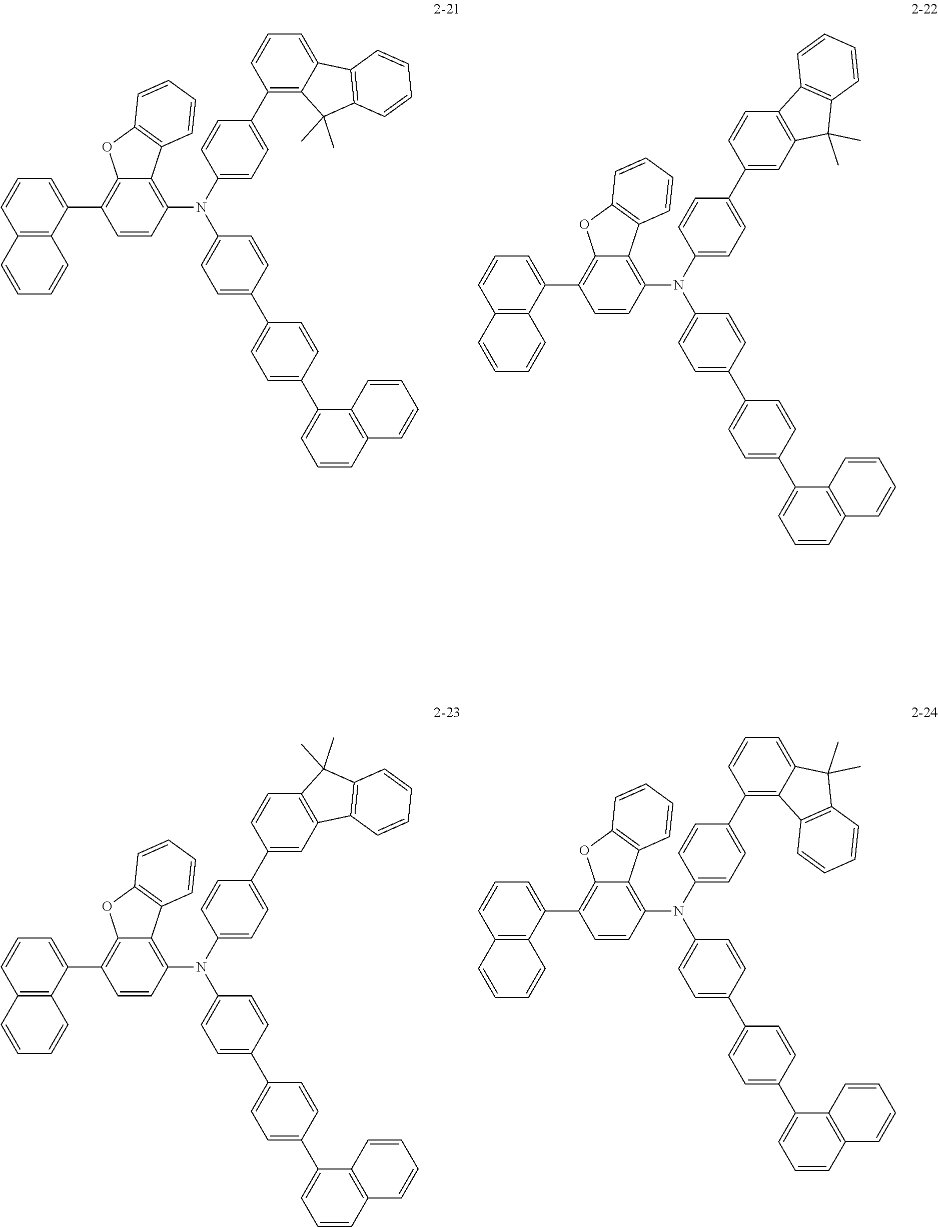 [SMILES: CC1(C)c2ccccc2-c2cccc(-c3ccc(N(c4ccc(-c5ccc(-c6cccc7ccccc67)cc5)cc4)c4ccc(-c5cccc6ccccc56)c5oc6ccccc6c45)cc3)c21 ; CC1(C)c2ccccc2-c2ccc(-c3ccc(N(c4ccc(-c5ccc(-c6cccc7ccccc67)cc5)cc4)c4ccc(-c5cccc6ccccc56)c5oc6ccccc6c45)cc3)cc21 ; CC1(C)c2ccccc2-c2cc(-c3ccc(N(c4ccc(-c5ccc(-c6cccc7ccccc67)cc5)cc4)c4ccc(-c5cccc6ccccc56)c5oc6ccccc6c45)cc3)ccc21 ; CC1(C)c2ccccc2-c2c(-c3ccc(N(c4ccc(-c5ccc(-c6cccc7ccccc67)cc5)cc4)c4ccc(-c5cccc6ccccc56)c5oc6ccccc6c45)cc3)cccc21]

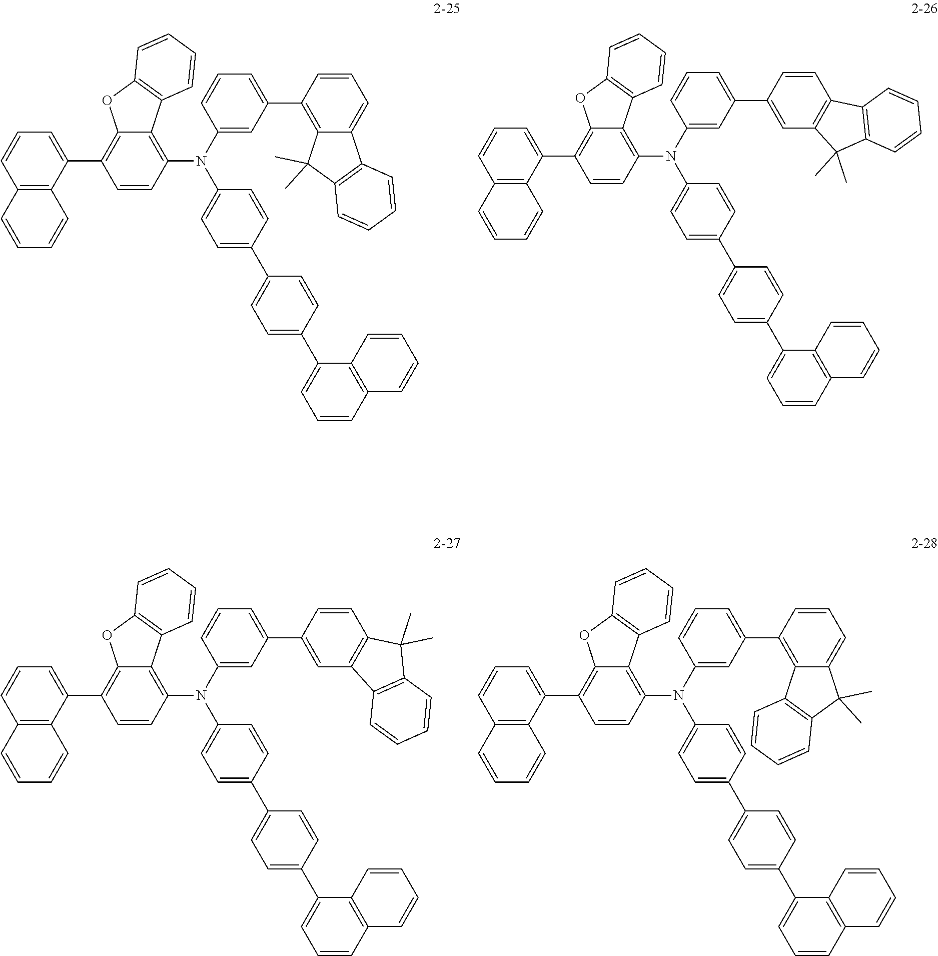 [SMILES: CC1(C)c2ccccc2-c2cccc(-c3cccc(N(c4ccc(-c5ccc(-c6cccc7ccccc67)cc5)cc4)c4ccc(-c5cccc6ccccc56)c5oc6ccccc6c45)c3)c21 ; CC1(C)c2ccccc2-c2ccc(-c3cccc(N(c4ccc(-c5ccc(-c6cccc7ccccc67)cc5)cc4)c4ccc(-c5cccc6ccccc56)c5oc6ccccc6c45)c3)cc21 ; CC1(C)c2ccccc2-c2cc(-c3cccc(N(c4ccc(-c5ccc(-c6cccc7ccccc67)cc5)cc4)c4ccc(-c5cccc6ccccc56)c5oc6ccccc6c45)c3)ccc21 ; CC1(C)c2ccccc2-c2c(-c3cccc(N(c4ccc(-c5ccc(-c6cccc7ccccc67)cc5)cc4)c4ccc(-c5cccc6ccccc56)c5oc6ccccc6c45)c3)cccc21]

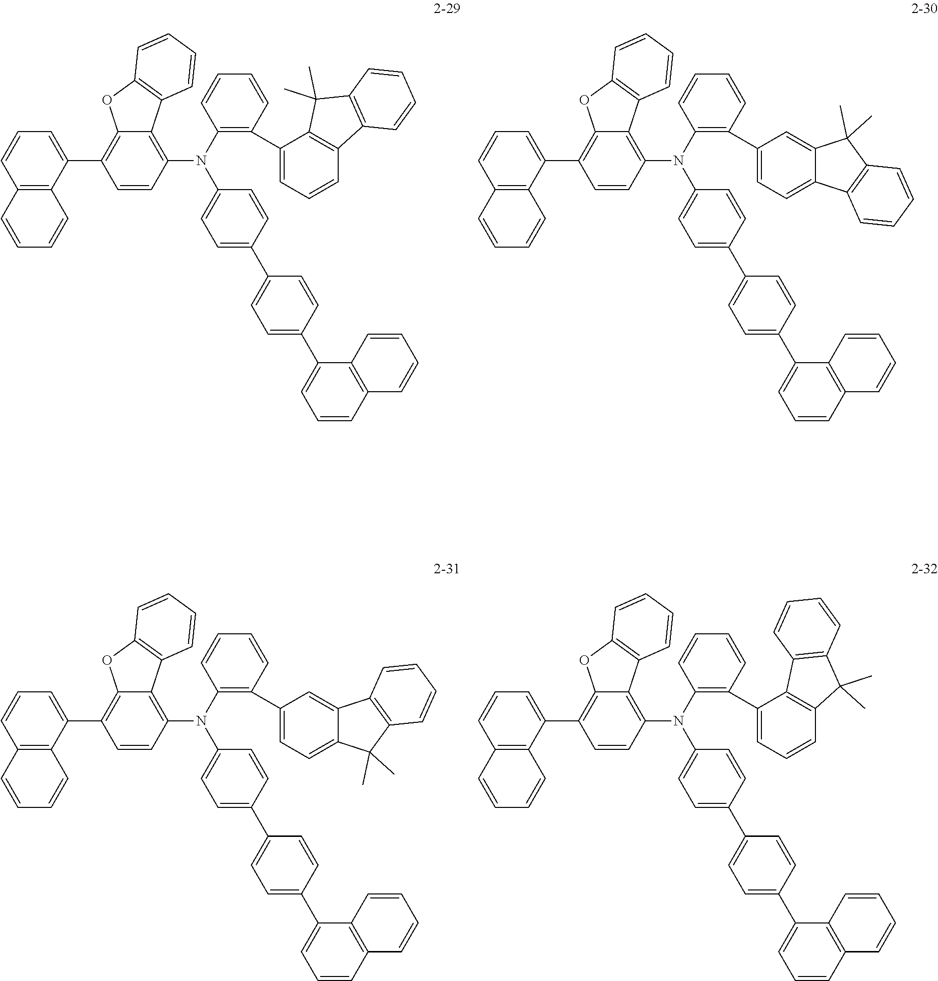 [SMILES: CC1(C)c2ccccc2-c2cccc(-c3ccccc3N(c3ccc(-c4ccc(-c5cccc6ccccc56)cc4)cc3)c3ccc(-c4cccc5ccccc45)c4oc5ccccc5c34)c21 ; CC1(C)c2ccccc2-c2ccc(-c3ccccc3N(c3ccc(-c4ccc(-c5cccc6ccccc56)cc4)cc3)c3ccc(-c4cccc5ccccc45)c4oc5ccccc5c34)cc21 ; CC1(C)c2ccccc2-c2cc(-c3ccccc3N(c3ccc(-c4ccc(-c5cccc6ccccc56)cc4)cc3)c3ccc(-c4cccc5ccccc45)c4oc5ccccc5c34)ccc21 ; CC1(C)c2ccccc2-c2c(-c3ccccc3N(c3ccc(-c4ccc(-c5cccc6ccccc56)cc4)cc3)c3ccc(-c4cccc5ccccc45)c4oc5ccccc5c34)cccc21]

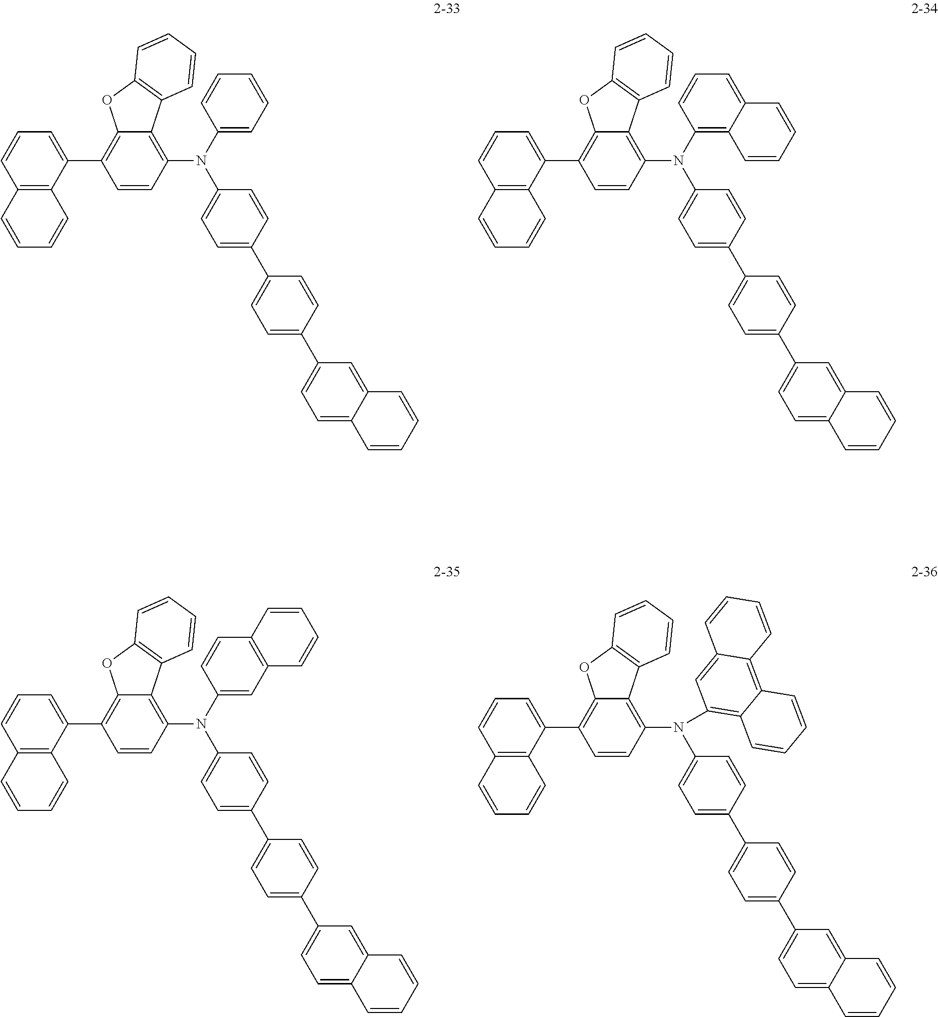 [SMILES: c1ccc(N(c2ccc(-c3ccc(-c4ccc5ccccc5c4)cc3)cc2)c2ccc(-c3cccc4ccccc34)c3oc4ccccc4c23)cc1 ; c1ccc2cc(-c3ccc(-c4ccc(N(c5cccc6ccccc56)c5ccc(-c6cccc7ccccc67)c6oc7ccccc7c56)cc4)cc3)ccc2c1 ; c1ccc2cc(-c3ccc(-c4ccc(N(c5ccc6ccccc6c5)c5ccc(-c6cccc7ccccc67)c6oc7ccccc7c56)cc4)cc3)ccc2c1 ; c1ccc2cc(-c3ccc(-c4ccc(N(c5cc6ccccc6c6ccccc56)c5ccc(-c6cccc7ccccc67)c6oc7ccccc7c56)cc4)cc3)ccc2c1]

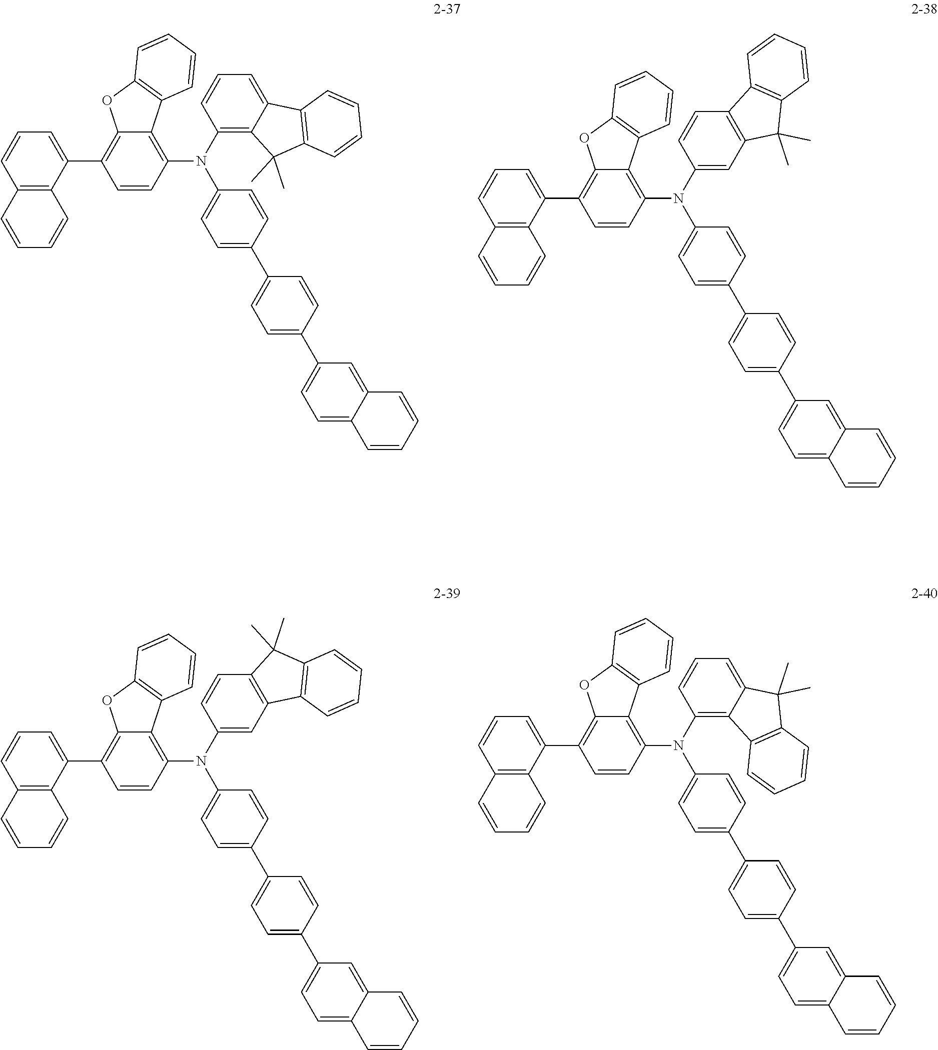 [SMILES: CC1(C)c2ccccc2-c2cccc(N(c3ccc(-c4ccc(-c5ccc6ccccc6c5)cc4)cc3)c3ccc(-c4cccc5ccccc45)c4oc5ccccc5c34)c21 ; CC1(C)c2ccccc2-c2ccc(N(c3ccc(-c4ccc(-c5ccc6ccccc6c5)cc4)cc3)c3ccc(-c4cccc5ccccc45)c4oc5ccccc5c34)cc21 ; CC1(C)c2ccccc2-c2cc(N(c3ccc(-c4ccc(-c5ccc6ccccc6c5)cc4)cc3)c3ccc(-c4cccc5ccccc45)c4oc5ccccc5c34)ccc21 ; CC1(C)c2ccccc2-c2c(N(c3ccc(-c4ccc(-c5ccc6ccccc6c5)cc4)cc3)c3ccc(-c4cccc5ccccc45)c4oc5ccccc5c34)cccc21]

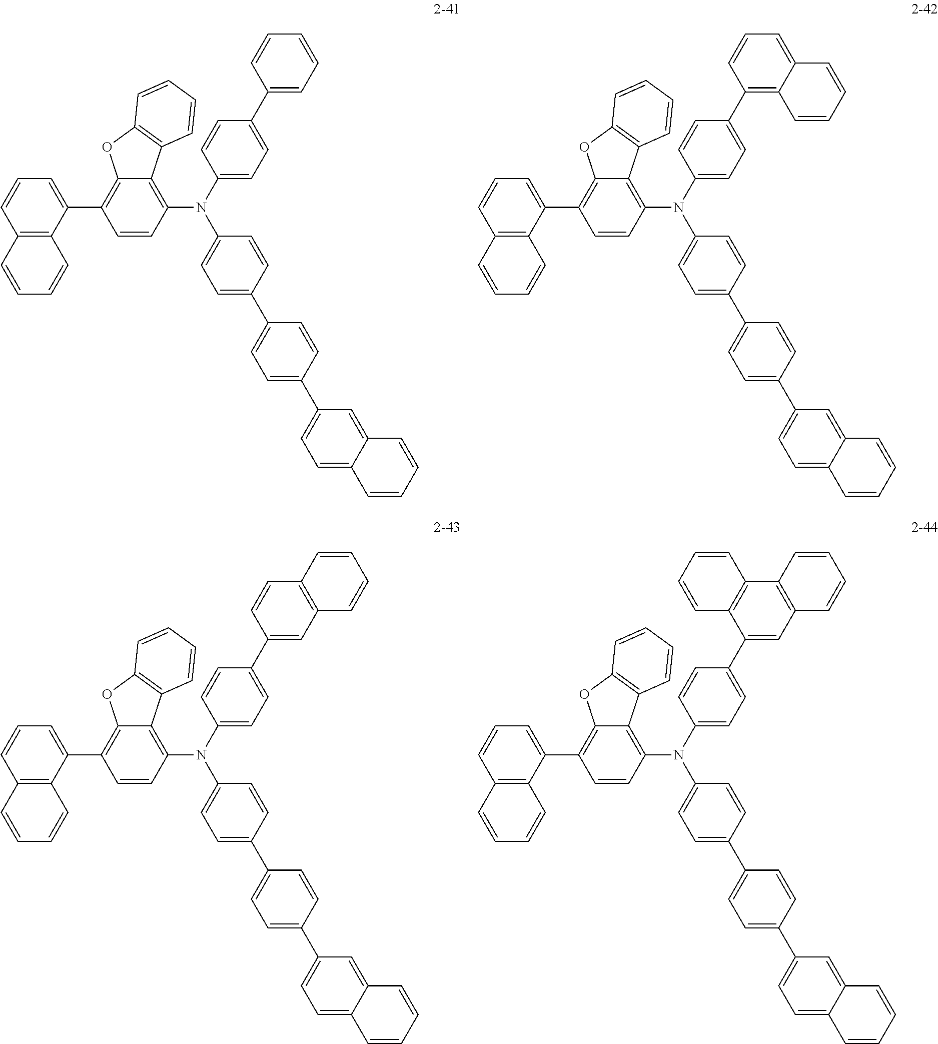 [SMILES: c1ccc(-c2ccc(N(c3ccc(-c4ccc(-c5ccc6ccccc6c5)cc4)cc3)c3ccc(-c4cccc5ccccc45)c4oc5ccccc5c34)cc2)cc1 ; c1ccc2cc(-c3ccc(-c4ccc(N(c5ccc(-c6cccc7ccccc67)cc5)c5ccc(-c6cccc7ccccc67)c6oc7ccccc7c56)cc4)cc3)ccc2c1 ; c1ccc2cc(-c3ccc(-c4ccc(N(c5ccc(-c6ccc7ccccc7c6)cc5)c5ccc(-c6cccc7ccccc67)c6oc7ccccc7c56)cc4)cc3)ccc2c1 ; c1ccc2cc(-c3ccc(-c4ccc(N(c5ccc(-c6cc7ccccc7c7ccccc67)cc5)c5ccc(-c6cccc7ccccc67)c6oc7ccccc7c56)cc4)cc3)ccc2c1]

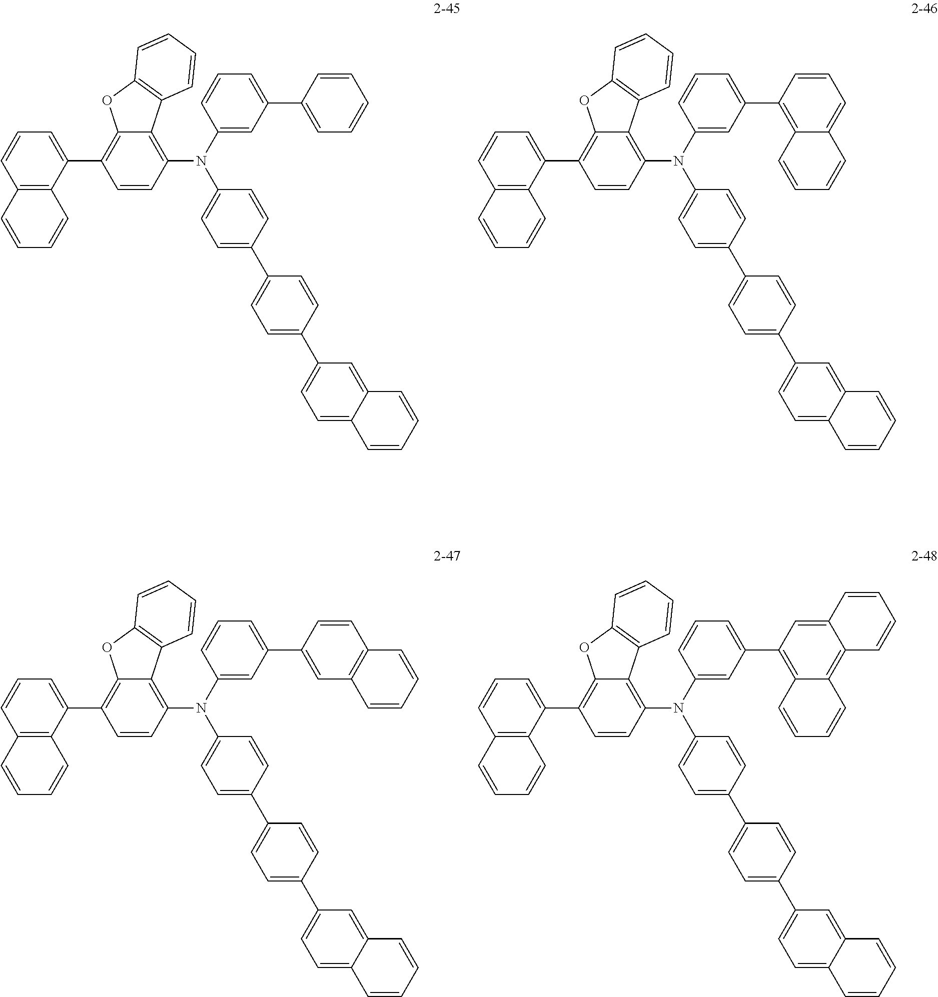 [SMILES: c1ccc(-c2cccc(N(c3ccc(-c4ccc(-c5ccc6ccccc6c5)cc4)cc3)c3ccc(-c4cccc5ccccc45)c4oc5ccccc5c34)c2)cc1 ; c1cc(-c2cccc3ccccc23)cc(N(c2ccc(-c3ccc(-c4ccc5ccccc5c4)cc3)cc2)c2ccc(-c3cccc4ccccc34)c3oc4ccccc4c23)c1 ; c1cc(-c2ccc3ccccc3c2)cc(N(c2ccc(-c3ccc(-c4ccc5ccccc5c4)cc3)cc2)c2ccc(-c3cccc4ccccc34)c3oc4ccccc4c23)c1 ; c1cc(-c2cc3ccccc3c3ccccc23)cc(N(c2ccc(-c3ccc(-c4ccc5ccccc5c4)cc3)cc2)c2ccc(-c3cccc4ccccc34)c3oc4ccccc4c23)c1]

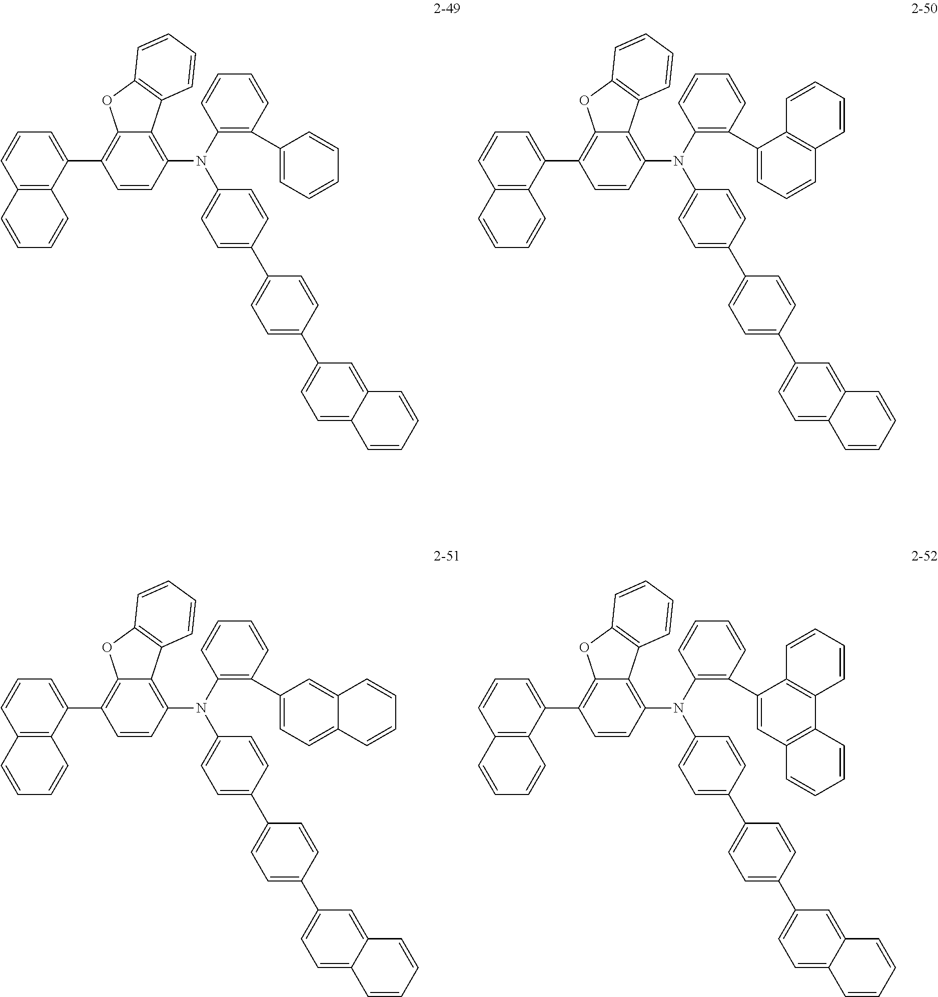 [SMILES: c1ccc(-c2ccccc2N(c2ccc(-c3ccc(-c4ccc5ccccc5c4)cc3)cc2)c2ccc(-c3cccc4ccccc34)c3oc4ccccc4c23)cc1 ; c1ccc(N(c2ccc(-c3ccc(-c4ccc5ccccc5c4)cc3)cc2)c2ccc(-c3cccc4ccccc34)c3oc4ccccc4c23)c(-c2cccc3ccccc23)c1 ; c1ccc(N(c2ccc(-c3ccc(-c4ccc5ccccc5c4)cc3)cc2)c2ccc(-c3cccc4ccccc34)c3oc4ccccc4c23)c(-c2ccc3ccccc3c2)c1 ; c1ccc(N(c2ccc(-c3ccc(-c4ccc5ccccc5c4)cc3)cc2)c2ccc(-c3cccc4ccccc34)c3oc4ccccc4c23)c(-c2cc3ccccc3c3ccccc23)c1]

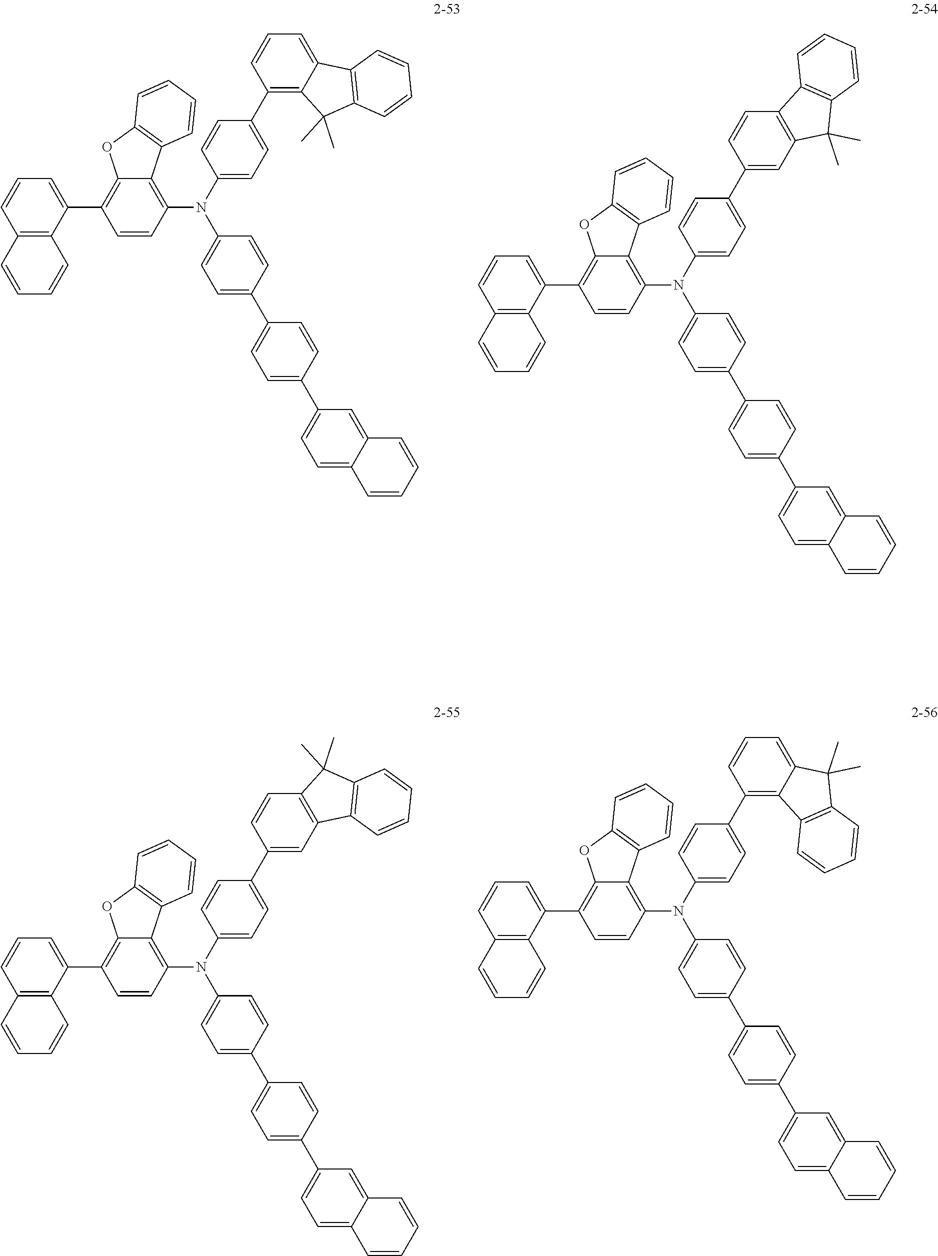 [SMILES: CC1(C)c2ccccc2-c2cccc(-c3ccc(N(c4ccc(-c5ccc(-c6ccc7ccccc7c6)cc5)cc4)c4ccc(-c5cccc6ccccc56)c5oc6ccccc6c45)cc3)c21 ; CC1(C)c2ccccc2-c2ccc(-c3ccc(N(c4ccc(-c5ccc(-c6ccc7ccccc7c6)cc5)cc4)c4ccc(-c5cccc6ccccc56)c5oc6ccccc6c45)cc3)cc21 ; CC1(C)c2ccccc2-c2cc(-c3ccc(N(c4ccc(-c5ccc(-c6ccc7ccccc7c6)cc5)cc4)c4ccc(-c5cccc6ccccc56)c5oc6ccccc6c45)cc3)ccc21 ; CC1(C)c2ccccc2-c2c(-c3ccc(N(c4ccc(-c5ccc(-c6ccc7ccccc7c6)cc5)cc4)c4ccc(-c5cccc6ccccc56)c5oc6ccccc6c45)cc3)cccc21]

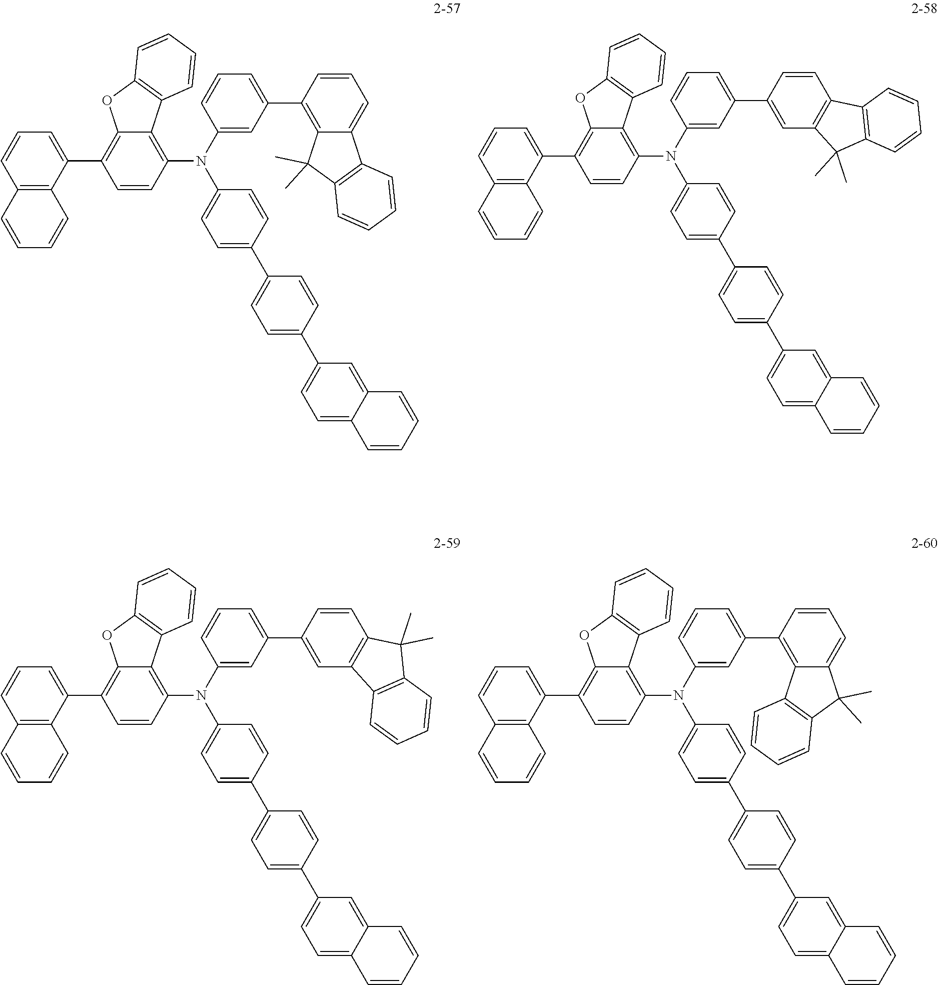 [SMILES: CC1(C)c2ccccc2-c2cccc(-c3cccc(N(c4ccc(-c5ccc(-c6ccc7ccccc7c6)cc5)cc4)c4ccc(-c5cccc6ccccc56)c5oc6ccccc6c45)c3)c21 ; CC1(C)c2ccccc2-c2ccc(-c3cccc(N(c4ccc(-c5ccc(-c6ccc7ccccc7c6)cc5)cc4)c4ccc(-c5cccc6ccccc56)c5oc6ccccc6c45)c3)cc21 ; CC1(C)c2ccccc2-c2cc(-c3cccc(N(c4ccc(-c5ccc(-c6ccc7ccccc7c6)cc5)cc4)c4ccc(-c5cccc6ccccc56)c5oc6ccccc6c45)c3)ccc21 ; CC1(C)c2ccccc2-c2c(-c3cccc(N(c4ccc(-c5ccc(-c6ccc7ccccc7c6)cc5)cc4)c4ccc(-c5cccc6ccccc56)c5oc6ccccc6c45)c3)cccc21]

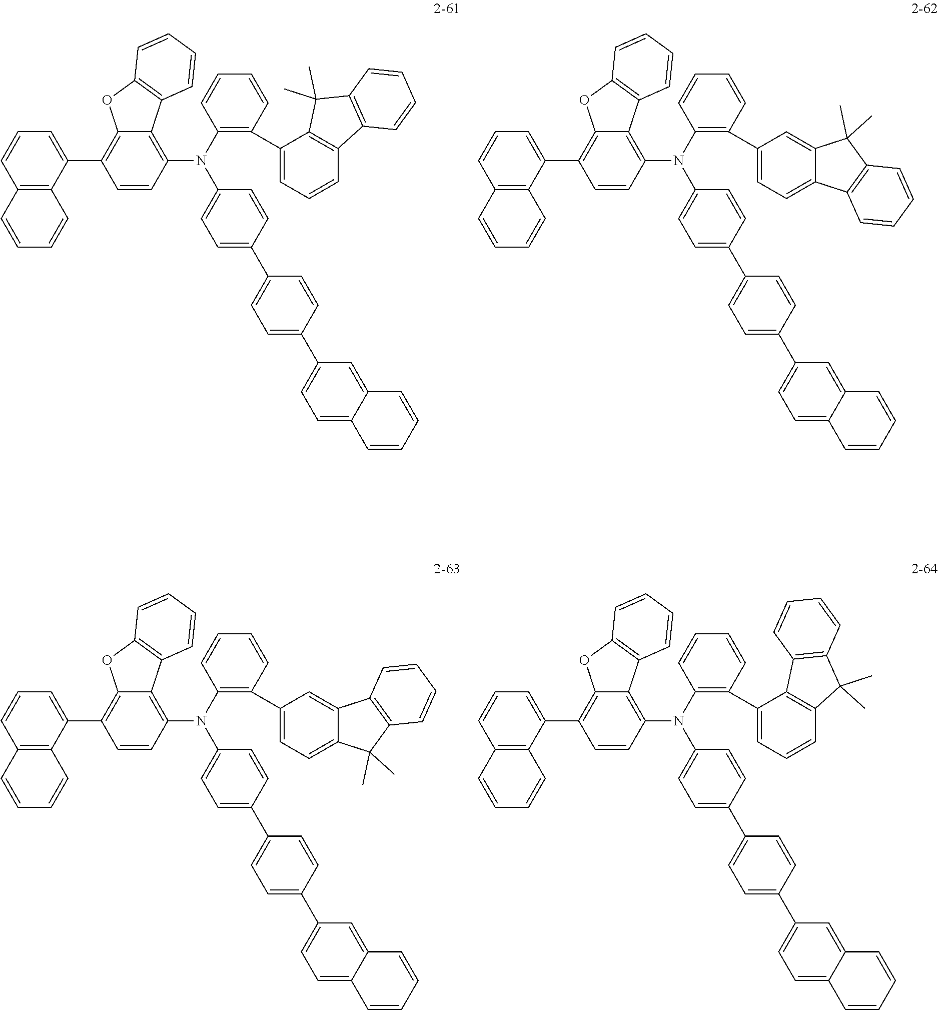 [SMILES: CC1(C)c2ccccc2-c2cccc(-c3ccccc3N(c3ccc(-c4ccc(-c5ccc6ccccc6c5)cc4)cc3)c3ccc(-c4cccc5ccccc45)c4oc5ccccc5c34)c21 ; CC1(C)c2ccccc2-c2ccc(-c3ccccc3N(c3ccc(-c4ccc(-c5ccc6ccccc6c5)cc4)cc3)c3ccc(-c4cccc5ccccc45)c4oc5ccccc5c34)cc21 ; CC1(C)c2ccccc2-c2cc(-c3ccccc3N(c3ccc(-c4ccc(-c5ccc6ccccc6c5)cc4)cc3)c3ccc(-c4cccc5ccccc45)c4oc5ccccc5c34)ccc21 ; CC1(C)c2ccccc2-c2c(-c3ccccc3N(c3ccc(-c4ccc(-c5ccc6ccccc6c5)cc4)cc3)c3ccc(-c4cccc5ccccc45)c4oc5ccccc5c34)cccc21]

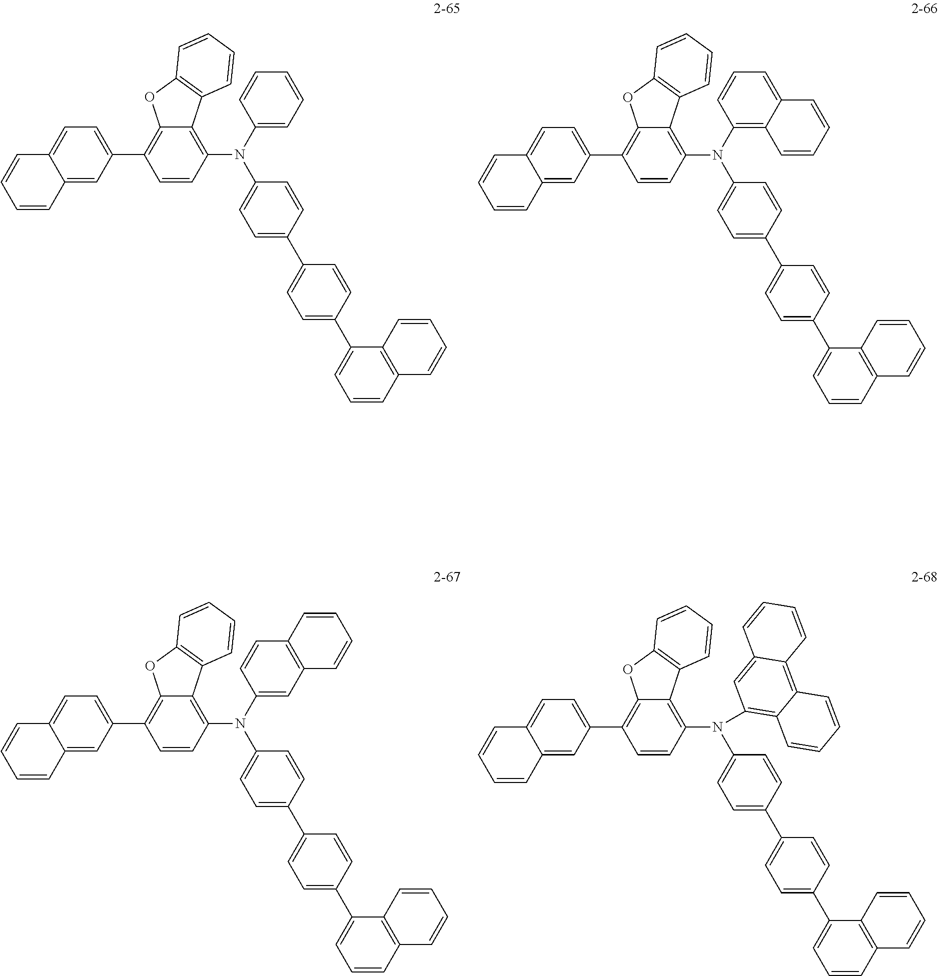 [SMILES: c1ccc(N(c2ccc(-c3ccc(-c4cccc5ccccc45)cc3)cc2)c2ccc(-c3ccc4ccccc4c3)c3oc4ccccc4c23)cc1 ; c1ccc2cc(-c3ccc(N(c4ccc(-c5ccc(-c6cccc7ccccc67)cc5)cc4)c4cccc5ccccc45)c4c3oc3ccccc34)ccc2c1 ; c1ccc2cc(-c3ccc(N(c4ccc(-c5ccc(-c6cccc7ccccc67)cc5)cc4)c4ccc5ccccc5c4)c4c3oc3ccccc34)ccc2c1 ; c1ccc2cc(-c3ccc(N(c4ccc(-c5ccc(-c6cccc7ccccc67)cc5)cc4)c4cc5ccccc5c5ccccc45)c4c3oc3ccccc34)ccc2c1]

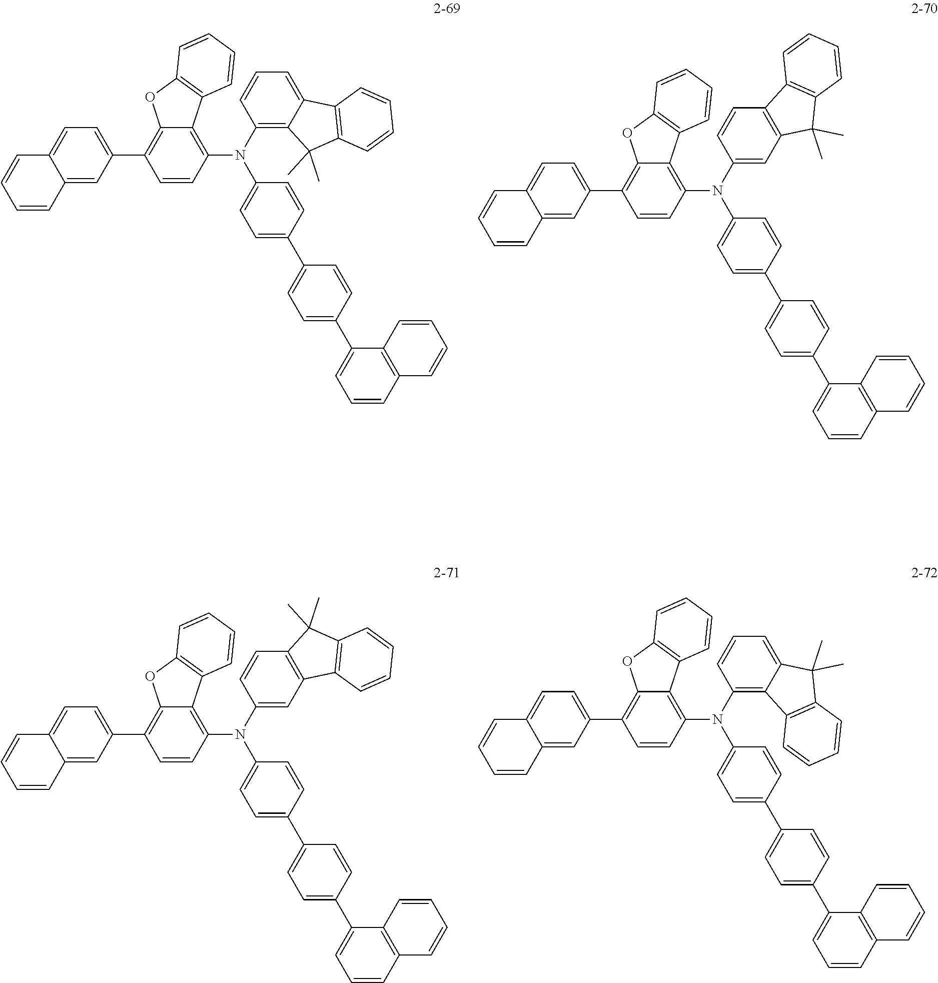 [SMILES: CC1(C)c2ccccc2-c2cccc(N(c3ccc(-c4ccc(-c5cccc6ccccc56)cc4)cc3)c3ccc(-c4ccc5ccccc5c4)c4oc5ccccc5c34)c21 ; CC1(C)c2ccccc2-c2ccc(N(c3ccc(-c4ccc(-c5cccc6ccccc56)cc4)cc3)c3ccc(-c4ccc5ccccc5c4)c4oc5ccccc5c34)cc21 ; CC1(C)c2ccccc2-c2cc(N(c3ccc(-c4ccc(-c5cccc6ccccc56)cc4)cc3)c3ccc(-c4ccc5ccccc5c4)c4oc5ccccc5c34)ccc21 ; CC1(C)c2ccccc2-c2c(N(c3ccc(-c4ccc(-c5cccc6ccccc56)cc4)cc3)c3ccc(-c4ccc5ccccc5c4)c4oc5ccccc5c34)cccc21]

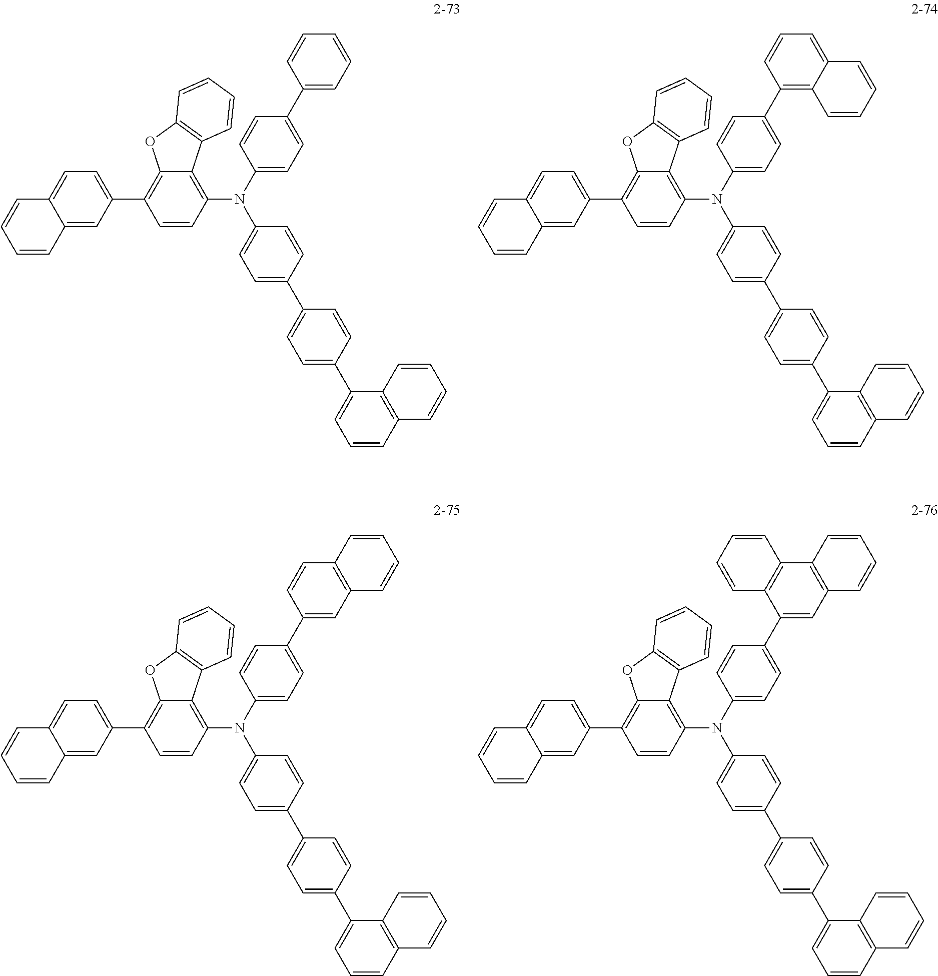 [SMILES: c1ccc(-c2ccc(N(c3ccc(-c4ccc(-c5cccc6ccccc56)cc4)cc3)c3ccc(-c4ccc5ccccc5c4)c4oc5ccccc5c34)cc2)cc1 ; c1ccc2cc(-c3ccc(N(c4ccc(-c5ccc(-c6cccc7ccccc67)cc5)cc4)c4ccc(-c5cccc6ccccc56)cc4)c4c3oc3ccccc34)ccc2c1 ; c1ccc2cc(-c3ccc(N(c4ccc(-c5ccc(-c6cccc7ccccc67)cc5)cc4)c4ccc(-c5ccc6ccccc6c5)c5oc6ccccc6c45)cc3)ccc2c1 ; c1ccc2cc(-c3ccc(N(c4ccc(-c5ccc(-c6cccc7ccccc67)cc5)cc4)c4ccc(-c5cc6ccccc6c6ccccc56)cc4)c4c3oc3ccccc34)ccc2c1]

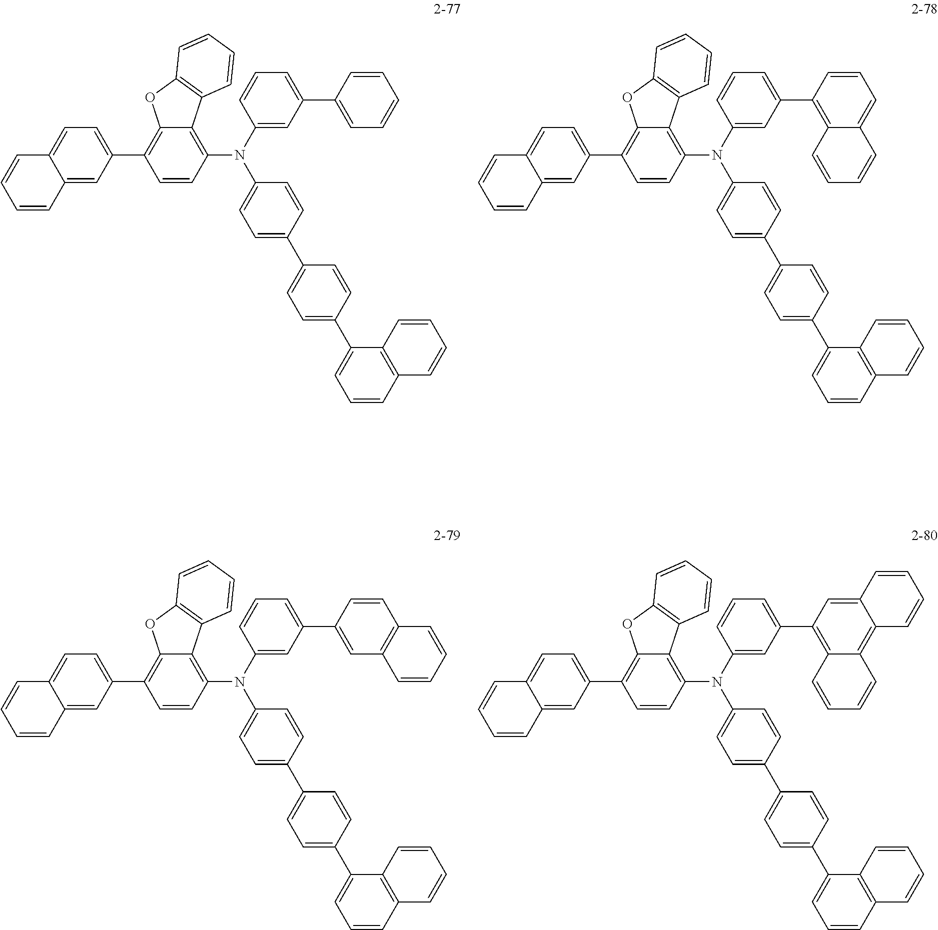 [SMILES: c1ccc(-c2cccc(N(c3ccc(-c4ccc(-c5cccc6ccccc56)cc4)cc3)c3ccc(-c4ccc5ccccc5c4)c4oc5ccccc5c34)c2)cc1 ; c1cc(-c2cccc3ccccc23)cc(N(c2ccc(-c3ccc(-c4cccc5ccccc45)cc3)cc2)c2ccc(-c3ccc4ccccc4c3)c3oc4ccccc4c23)c1 ; c1cc(-c2ccc3ccccc3c2)cc(N(c2ccc(-c3ccc(-c4cccc5ccccc45)cc3)cc2)c2ccc(-c3ccc4ccccc4c3)c3oc4ccccc4c23)c1 ; c1cc(-c2cc3ccccc3c3ccccc23)cc(N(c2ccc(-c3ccc(-c4cccc5ccccc45)cc3)cc2)c2ccc(-c3ccc4ccccc4c3)c3oc4ccccc4c23)c1]

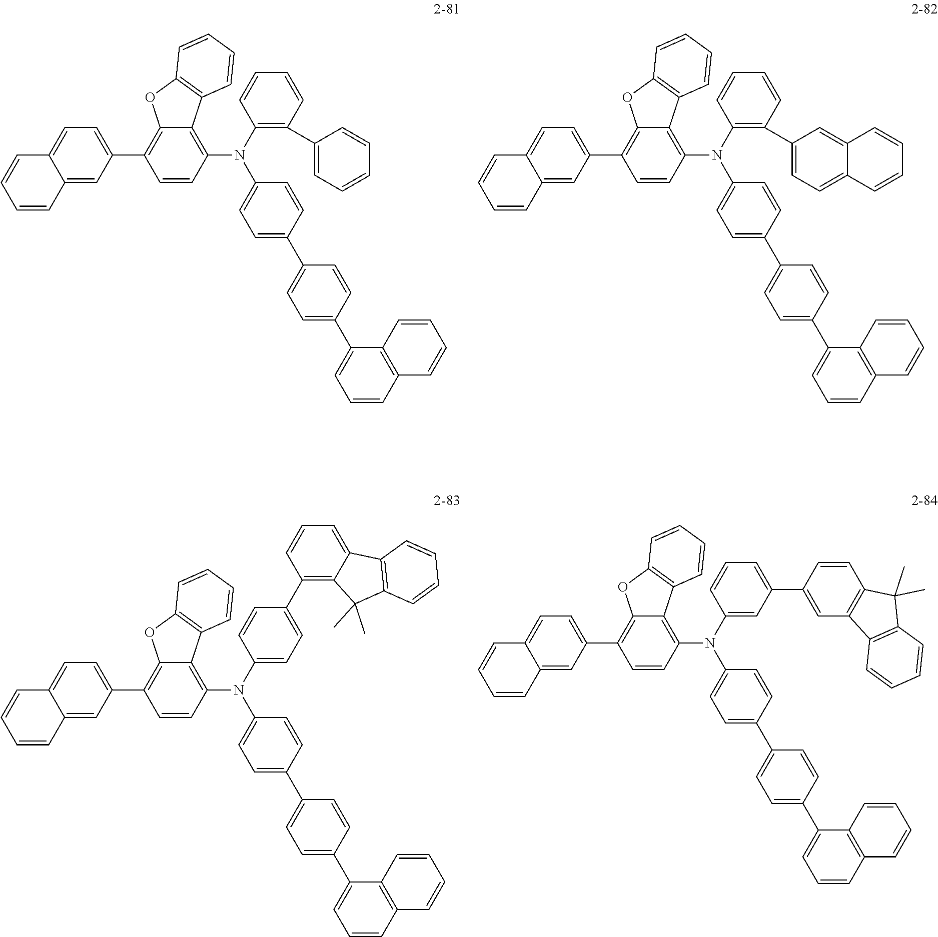 [SMILES: c1ccc(-c2ccccc2N(c2ccc(-c3ccc(-c4cccc5ccccc45)cc3)cc2)c2ccc(-c3ccc4ccccc4c3)c3oc4ccccc4c23)cc1 ; c1ccc(N(c2ccc(-c3ccc(-c4cccc5ccccc45)cc3)cc2)c2ccc(-c3ccc4ccccc4c3)c3oc4ccccc4c23)c(-c2ccc3ccccc3c2)c1 ; CC1(C)c2ccccc2-c2cccc(-c3ccc(N(c4ccc(-c5ccc(-c6cccc7ccccc67)cc5)cc4)c4ccc(-c5ccc6ccccc6c5)c5oc6ccccc6c45)cc3)c21 ; CC1(C)c2ccccc2-c2cc(-c3cccc(N(c4ccc(-c5ccc(-c6cccc7ccccc67)cc5)cc4)c4ccc(-c5ccc6ccccc6c5)c5oc6ccccc6c45)c3)ccc21]

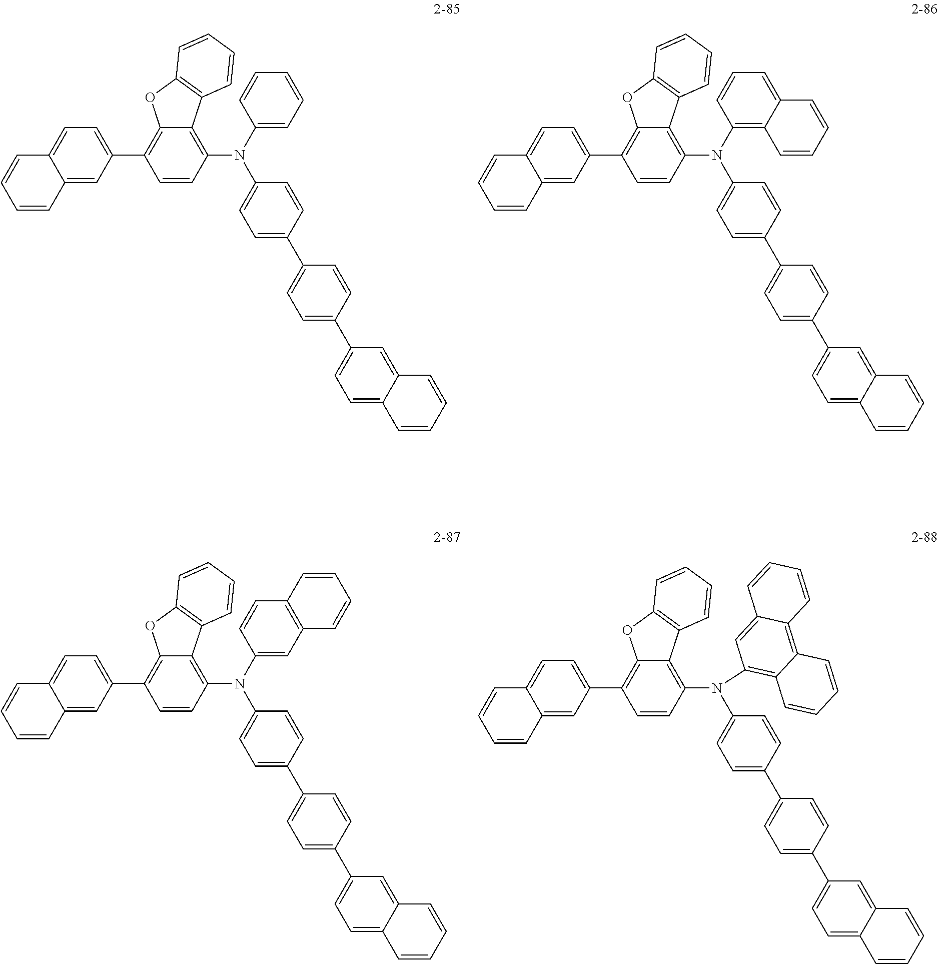 [SMILES: c1ccc(N(c2ccc(-c3ccc(-c4ccc5ccccc5c4)cc3)cc2)c2ccc(-c3ccc4ccccc4c3)c3oc4ccccc4c23)cc1 ; c1ccc2cc(-c3ccc(-c4ccc(N(c5cccc6ccccc56)c5ccc(-c6ccc7ccccc7c6)c6oc7ccccc7c56)cc4)cc3)ccc2c1 ; c1ccc2cc(-c3ccc(-c4ccc(N(c5ccc6ccccc6c5)c5ccc(-c6ccc7ccccc7c6)c6oc7ccccc7c56)cc4)cc3)ccc2c1 ; c1ccc2cc(-c3ccc(-c4ccc(N(c5cc6ccccc6c6ccccc56)c5ccc(-c6ccc7ccccc7c6)c6oc7ccccc7c56)cc4)cc3)ccc2c1]

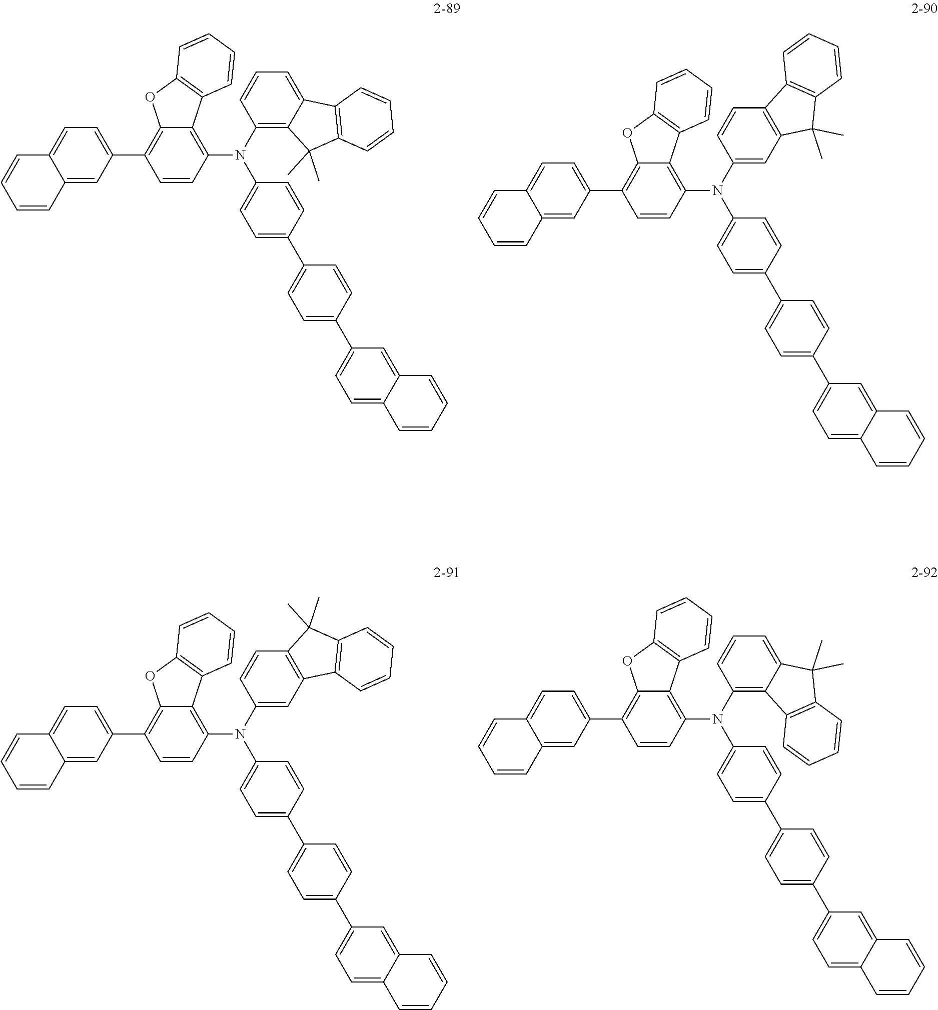 [SMILES: CC1(C)c2ccccc2-c2cccc(N(c3ccc(-c4ccc(-c5ccc6ccccc6c5)cc4)cc3)c3ccc(-c4ccc5ccccc5c4)c4oc5ccccc5c34)c21 ; CC1(C)c2ccccc2-c2ccc(N(c3ccc(-c4ccc(-c5ccc6ccccc6c5)cc4)cc3)c3ccc(-c4ccc5ccccc5c4)c4oc5ccccc5c34)cc21 ; CC1(C)c2ccccc2-c2cc(N(c3ccc(-c4ccc(-c5ccc6ccccc6c5)cc4)cc3)c3ccc(-c4ccc5ccccc5c4)c4oc5ccccc5c34)ccc21 ; CC1(C)c2ccccc2-c2c(N(c3ccc(-c4ccc(-c5ccc6ccccc6c5)cc4)cc3)c3ccc(-c4ccc5ccccc5c4)c4oc5ccccc5c34)cccc21]

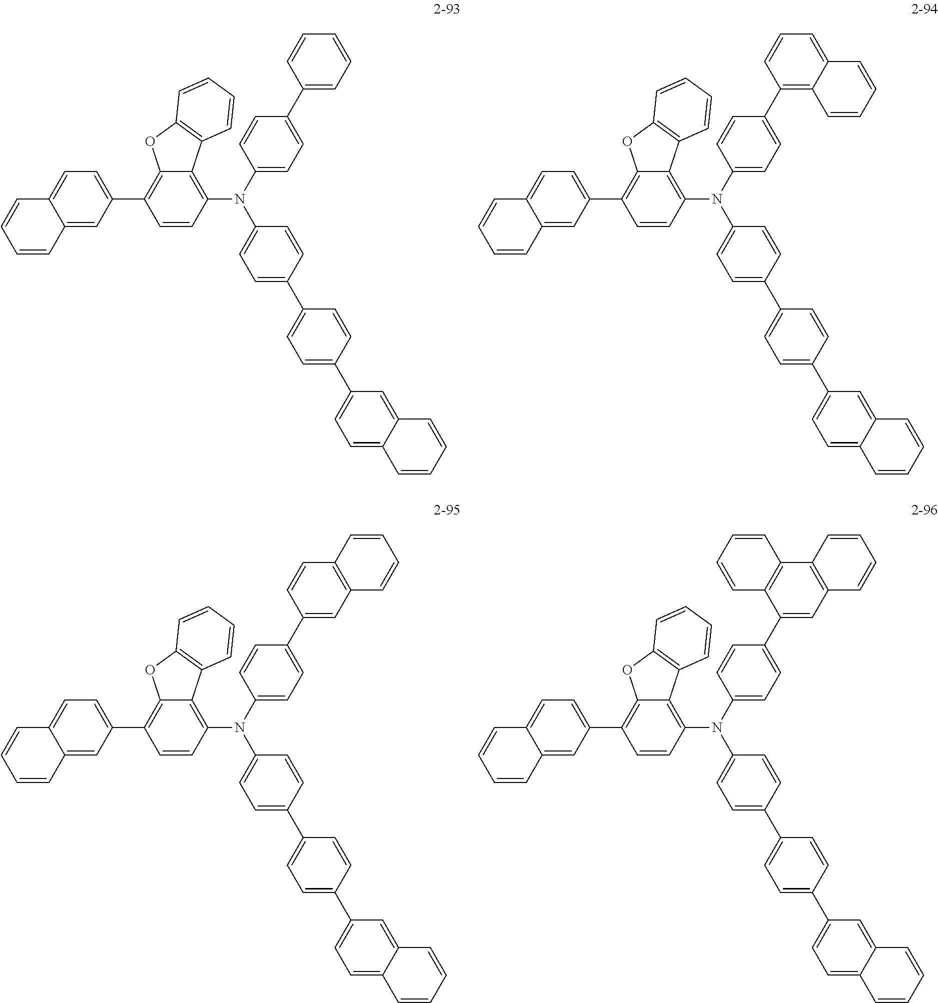 [SMILES: c1ccc(-c2ccc(N(c3ccc(-c4ccc(-c5ccc6ccccc6c5)cc4)cc3)c3ccc(-c4ccc5ccccc5c4)c4oc5ccccc5c34)cc2)cc1 ; c1ccc2cc(-c3ccc(-c4ccc(N(c5ccc(-c6cccc7ccccc67)cc5)c5ccc(-c6ccc7ccccc7c6)c6oc7ccccc7c56)cc4)cc3)ccc2c1 ; c1ccc2cc(-c3ccc(-c4ccc(N(c5ccc(-c6ccc7ccccc7c6)cc5)c5ccc(-c6ccc7ccccc7c6)c6oc7ccccc7c56)cc4)cc3)ccc2c1 ; c1ccc2cc(-c3ccc(-c4ccc(N(c5ccc(-c6cc7ccccc7c7ccccc67)cc5)c5ccc(-c6ccc7ccccc7c6)c6oc7ccccc7c56)cc4)cc3)ccc2c1]

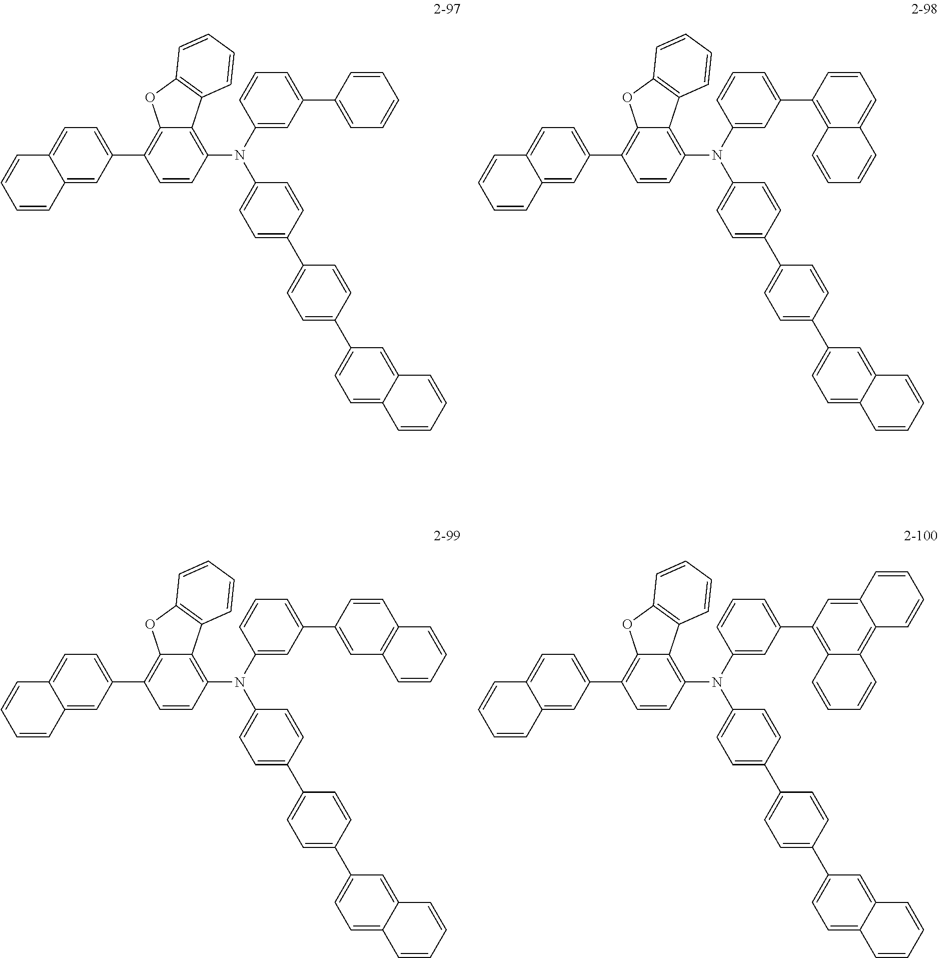 [SMILES: c1ccc(-c2cccc(N(c3ccc(-c4ccc(-c5ccc6ccccc6c5)cc4)cc3)c3ccc(-c4ccc5ccccc5c4)c4oc5ccccc5c34)c2)cc1 ; c1cc(-c2cccc3ccccc23)cc(N(c2ccc(-c3ccc(-c4ccc5ccccc5c4)cc3)cc2)c2ccc(-c3ccc4ccccc4c3)c3oc4ccccc4c23)c1 ; c1cc(-c2ccc3ccccc3c2)cc(N(c2ccc(-c3ccc(-c4ccc5ccccc5c4)cc3)cc2)c2ccc(-c3ccc4ccccc4c3)c3oc4ccccc4c23)c1 ; c1cc(-c2cc3ccccc3c3ccccc23)cc(N(c2ccc(-c3ccc(-c4ccc5ccccc5c4)cc3)cc2)c2ccc(-c3ccc4ccccc4c3)c3oc4ccccc4c23)c1]

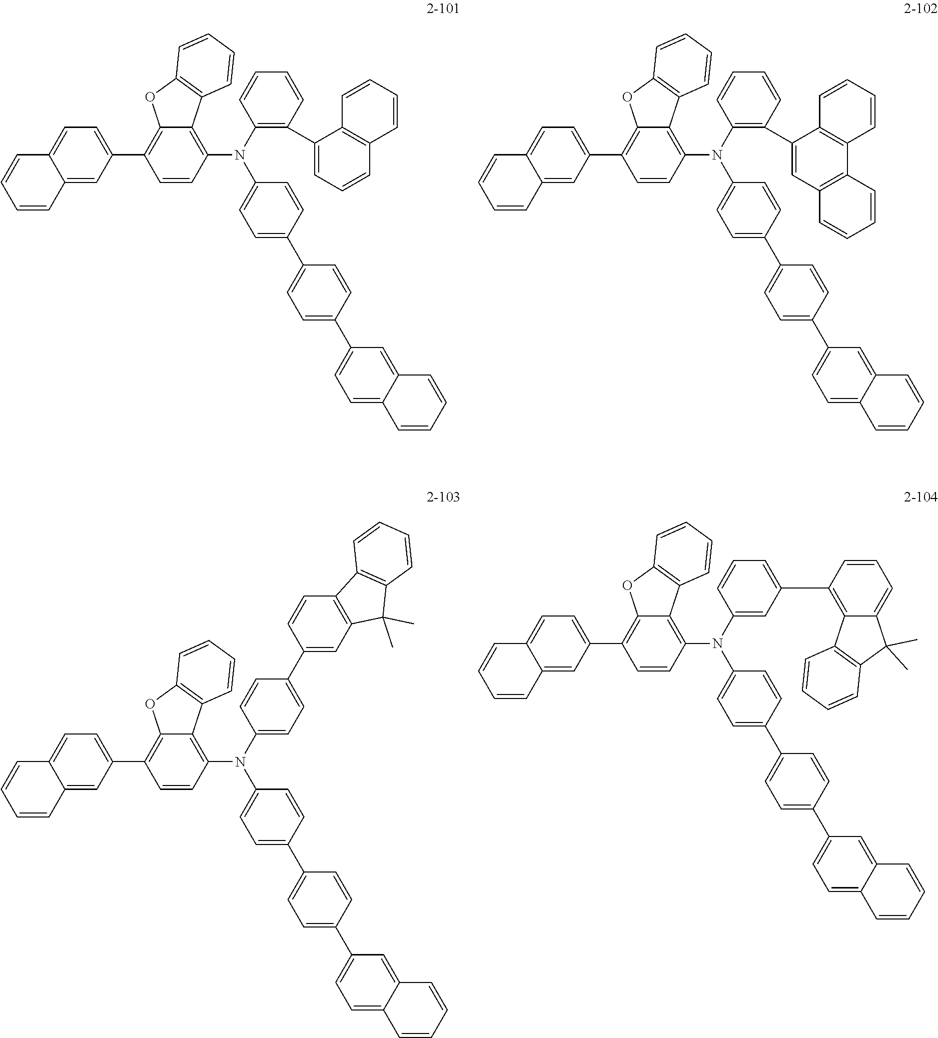 [SMILES: c1ccc(N(c2ccc(-c3ccc(-c4ccc5ccccc5c4)cc3)cc2)c2ccc(-c3ccc4ccccc4c3)c3oc4ccccc4c23)c(-c2cccc3ccccc23)c1 ; c1ccc(N(c2ccc(-c3ccc(-c4ccc5ccccc5c4)cc3)cc2)c2ccc(-c3ccc4ccccc4c3)c3oc4ccccc4c23)c(-c2cc3ccccc3c3ccccc23)c1 ; CC1(C)c2ccccc2-c2ccc(-c3ccc(N(c4ccc(-c5ccc(-c6ccc7ccccc7c6)cc5)cc4)c4ccc(-c5ccc6ccccc6c5)c5oc6ccccc6c45)cc3)cc21 ; CC1(C)c2ccccc2-c2c(-c3cccc(N(c4ccc(-c5ccc(-c6ccc7ccccc7c6)cc5)cc4)c4ccc(-c5ccc6ccccc6c5)c5oc6ccccc6c45)c3)cccc21]

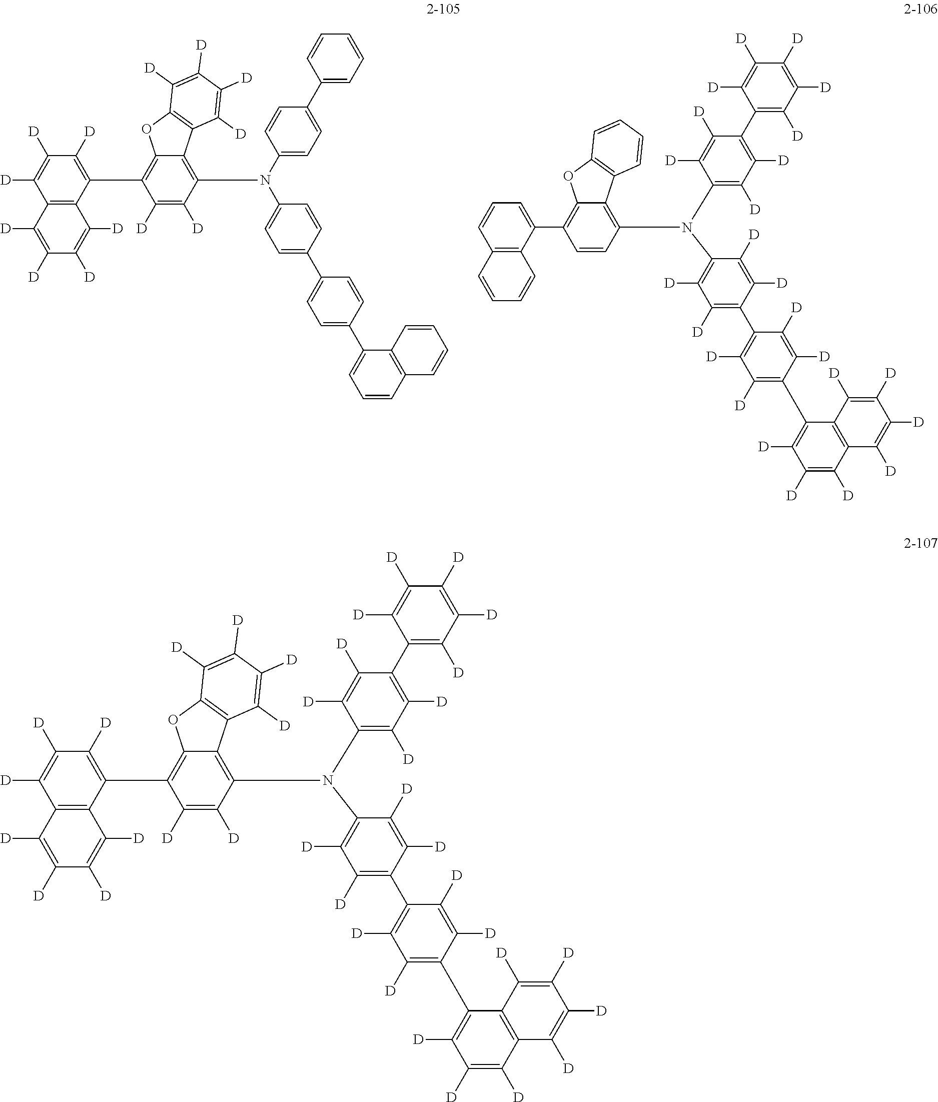

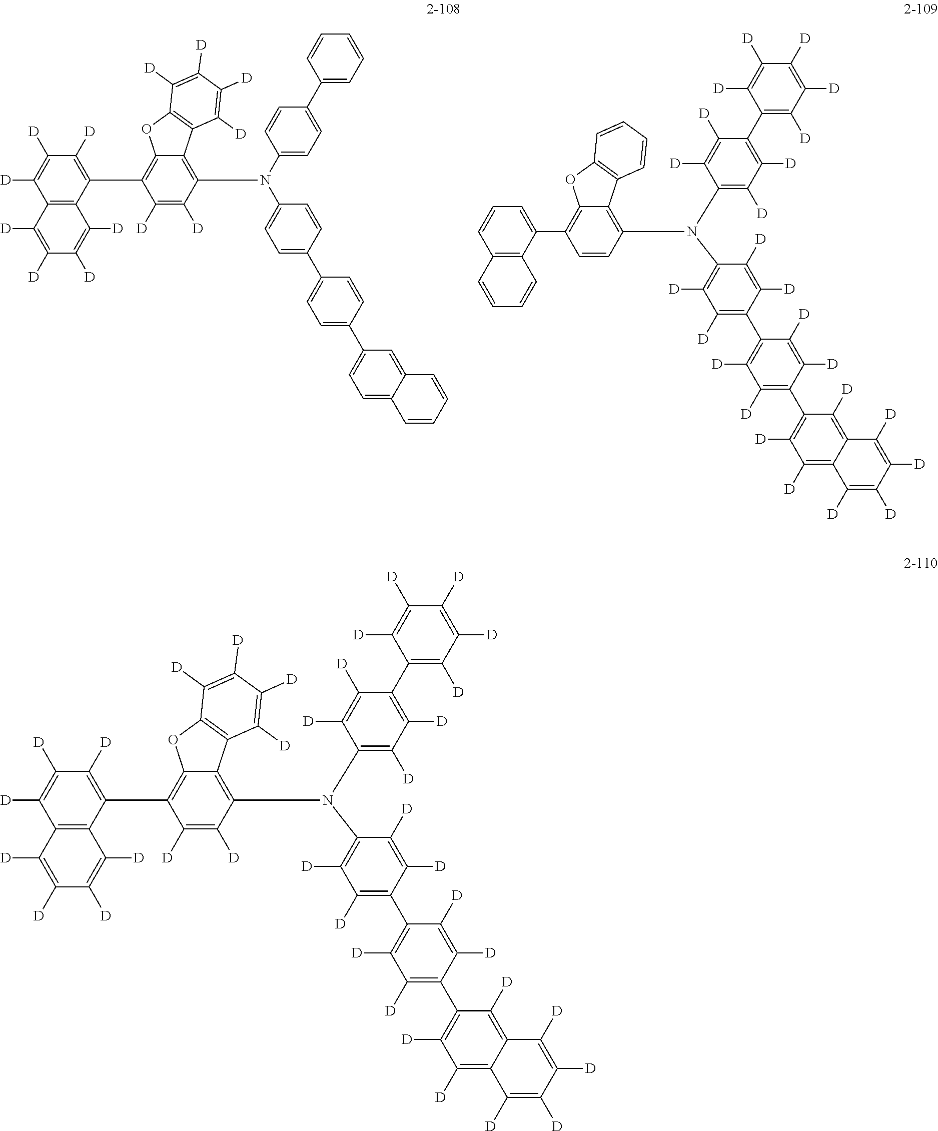

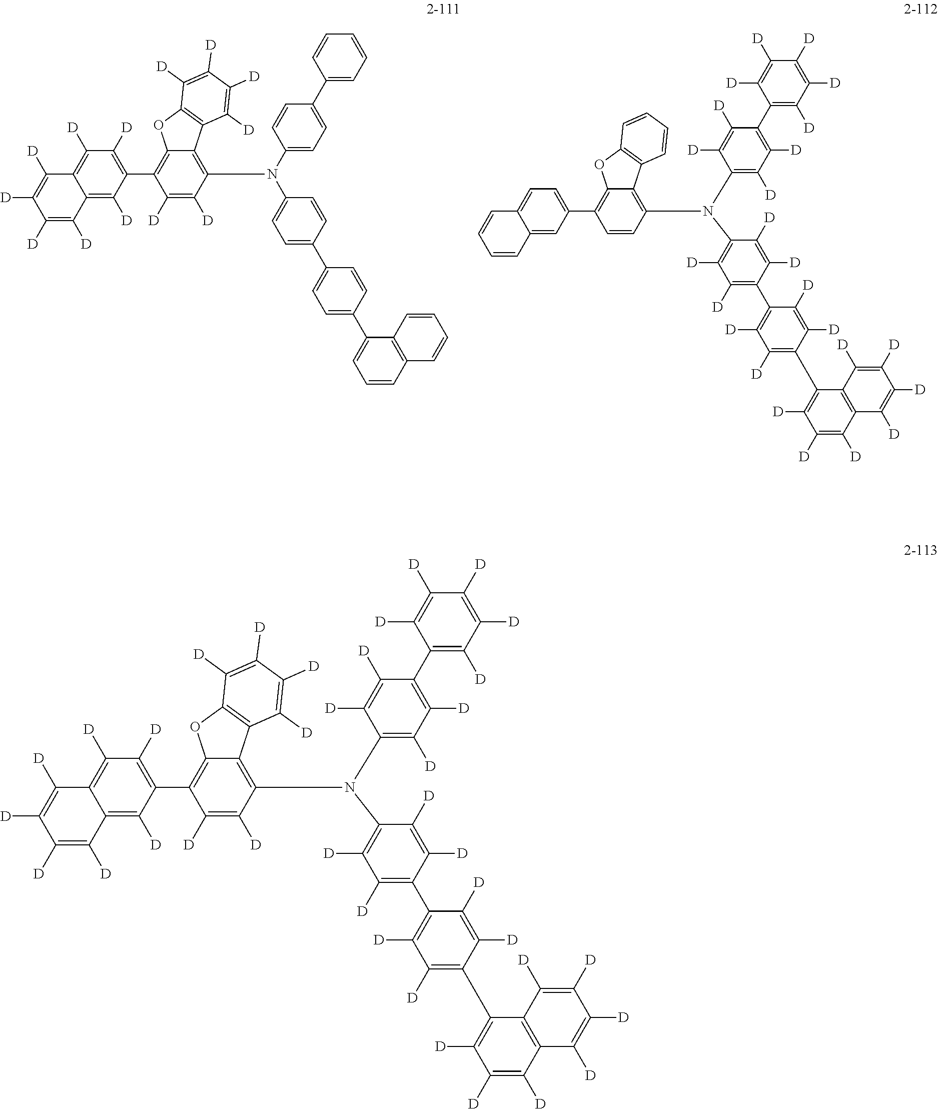

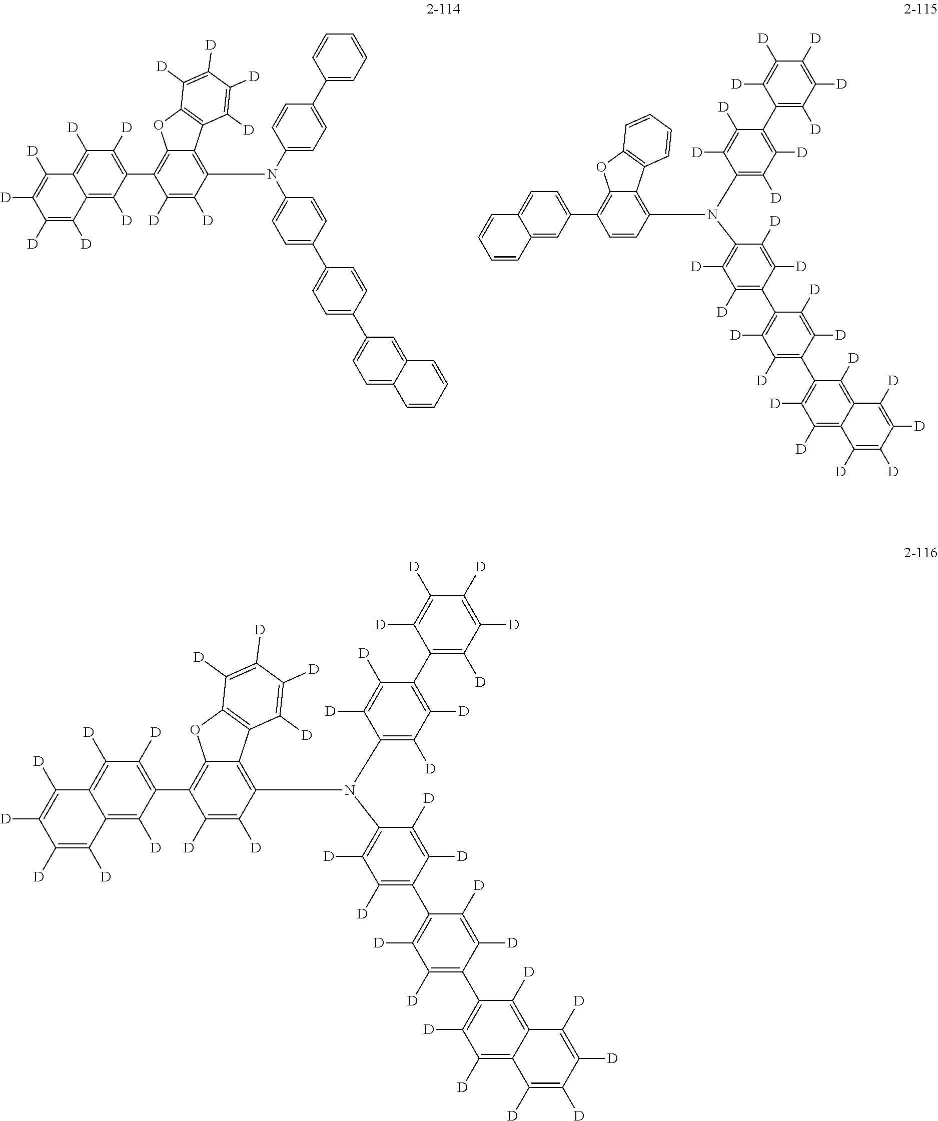

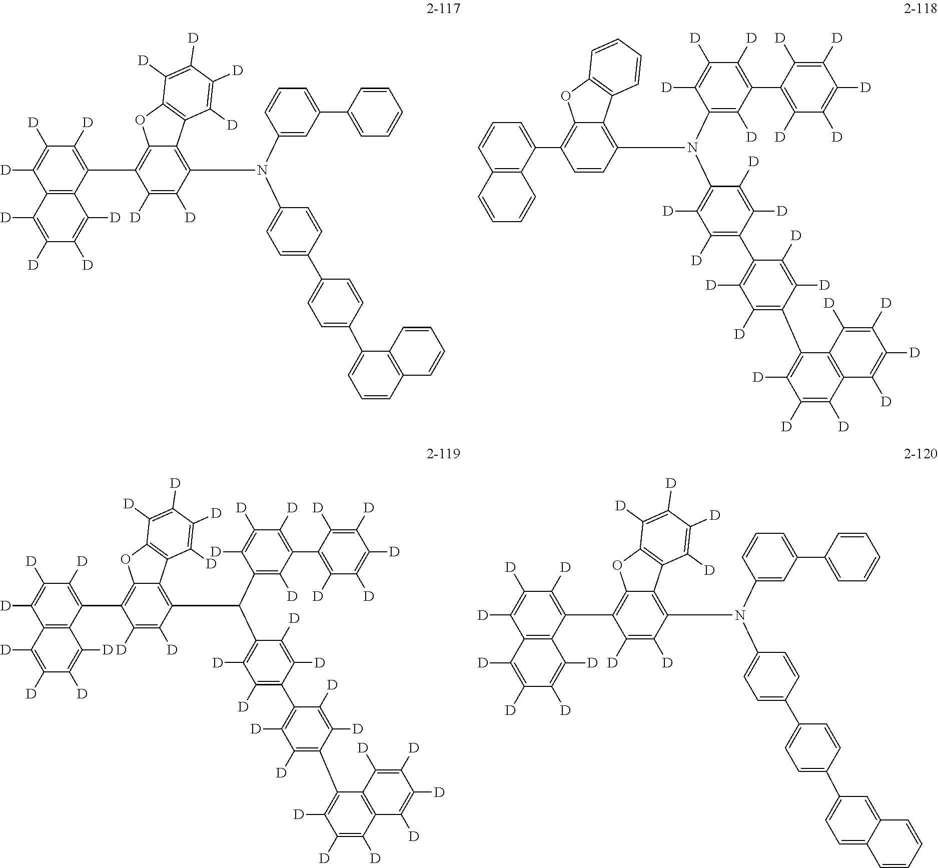

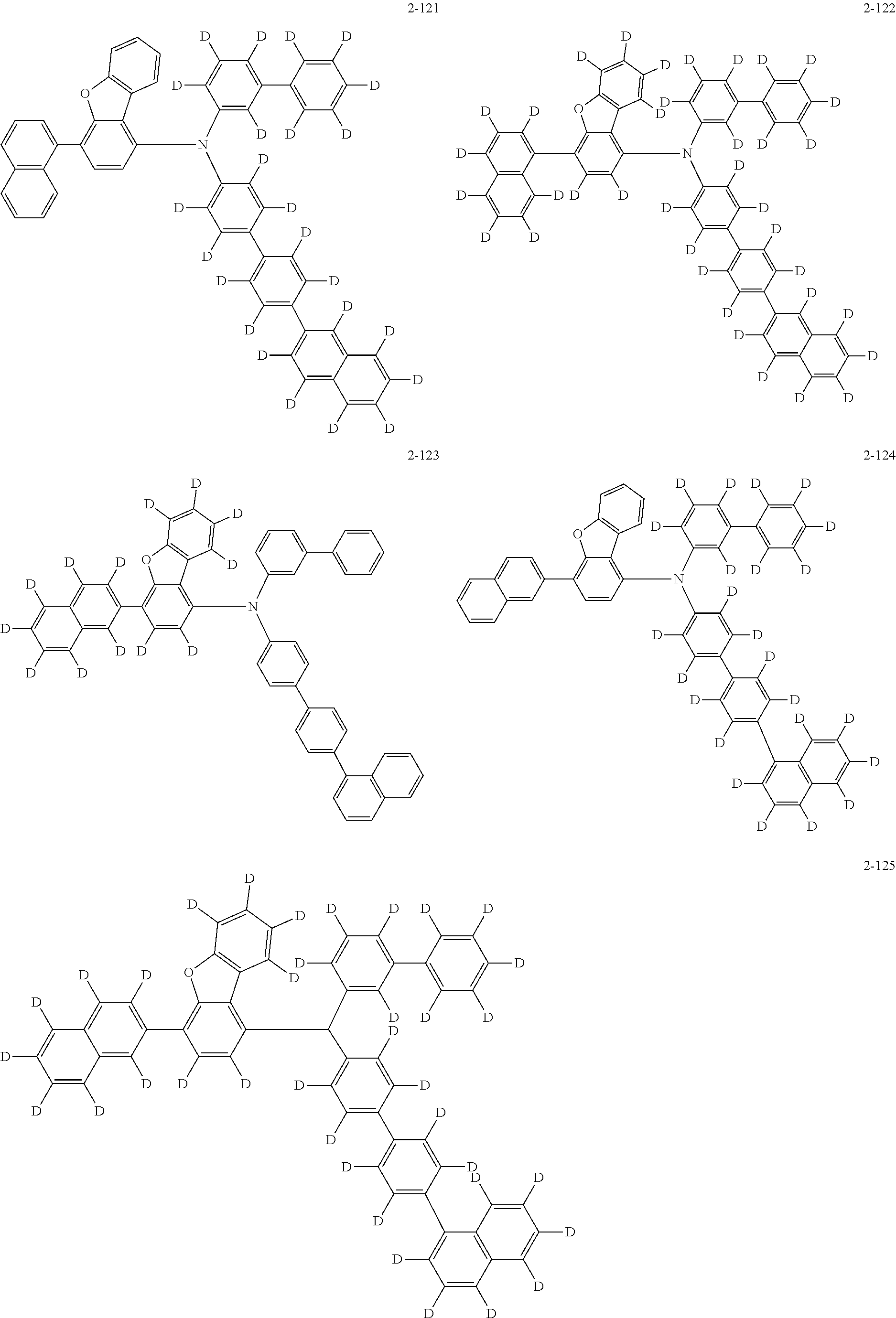

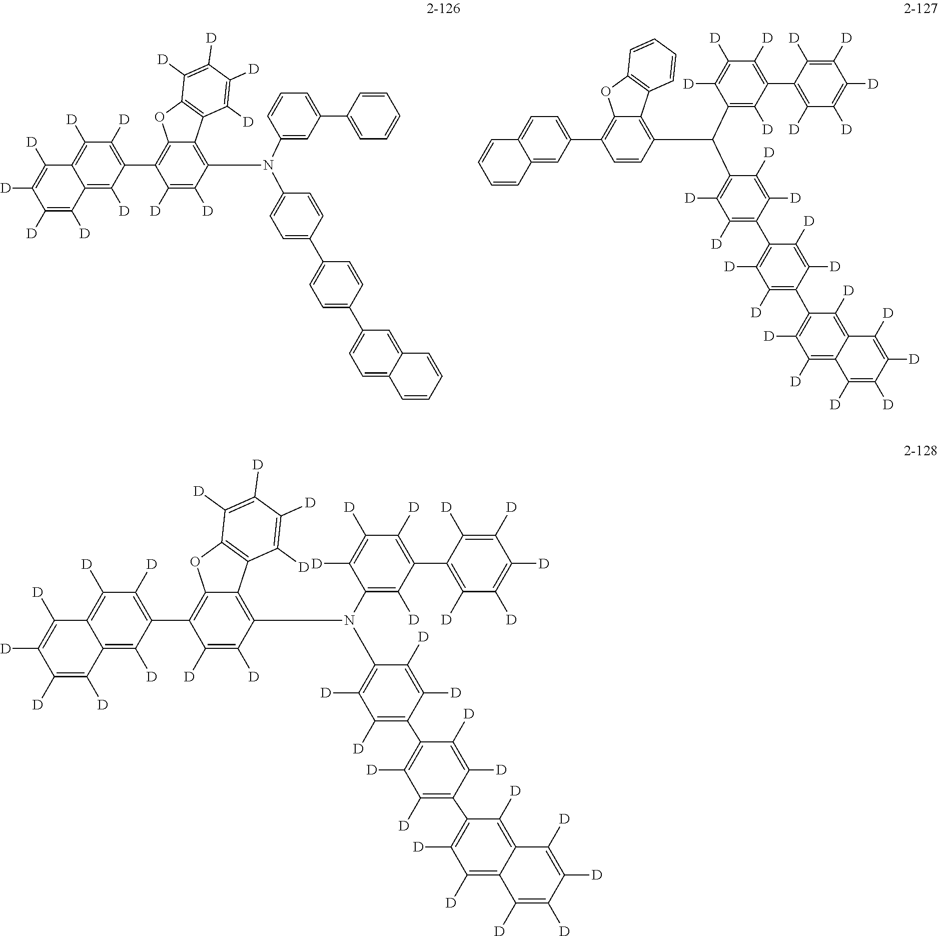

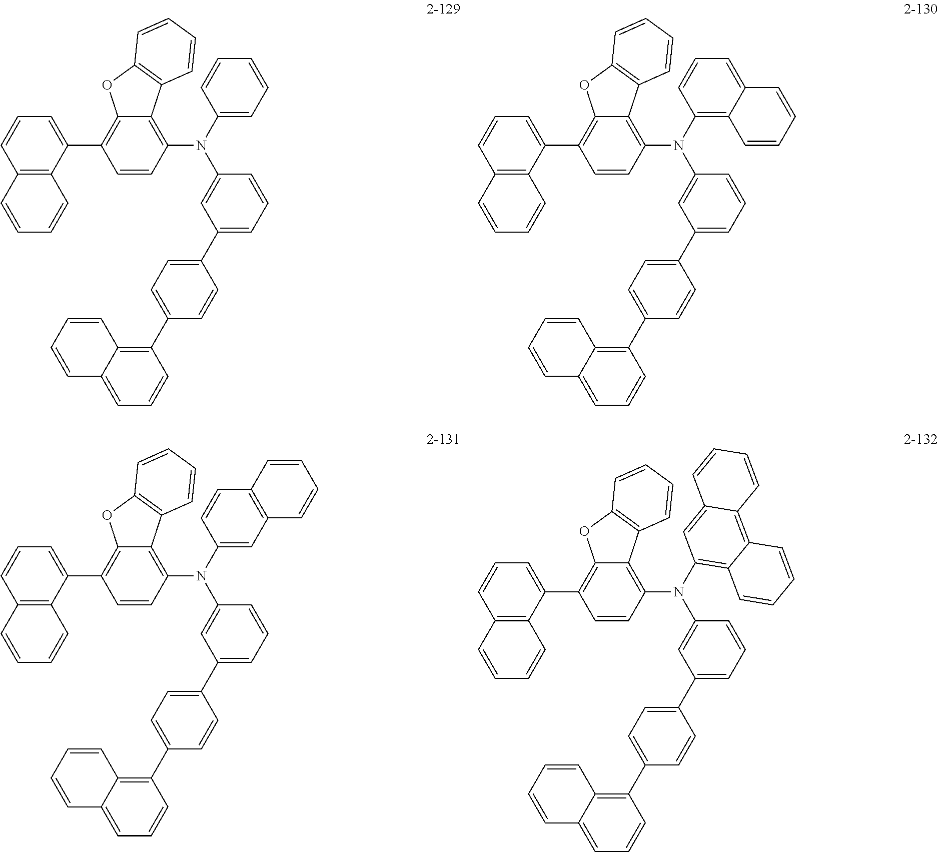 [SMILES: c1ccc(N(c2cccc(-c3ccc(-c4cccc5ccccc45)cc3)c2)c2ccc(-c3cccc4ccccc34)c3oc4ccccc4c23)cc1 ; c1cc(-c2ccc(-c3cccc4ccccc34)cc2)cc(N(c2cccc3ccccc23)c2ccc(-c3cccc4ccccc34)c3oc4ccccc4c23)c1 ; c1cc(-c2ccc(-c3cccc4ccccc34)cc2)cc(N(c2ccc3ccccc3c2)c2ccc(-c3cccc4ccccc34)c3oc4ccccc4c23)c1 ; c1cc(-c2ccc(-c3cccc4ccccc34)cc2)cc(N(c2cc3ccccc3c3ccccc23)c2ccc(-c3cccc4ccccc34)c3oc4ccccc4c23)c1]

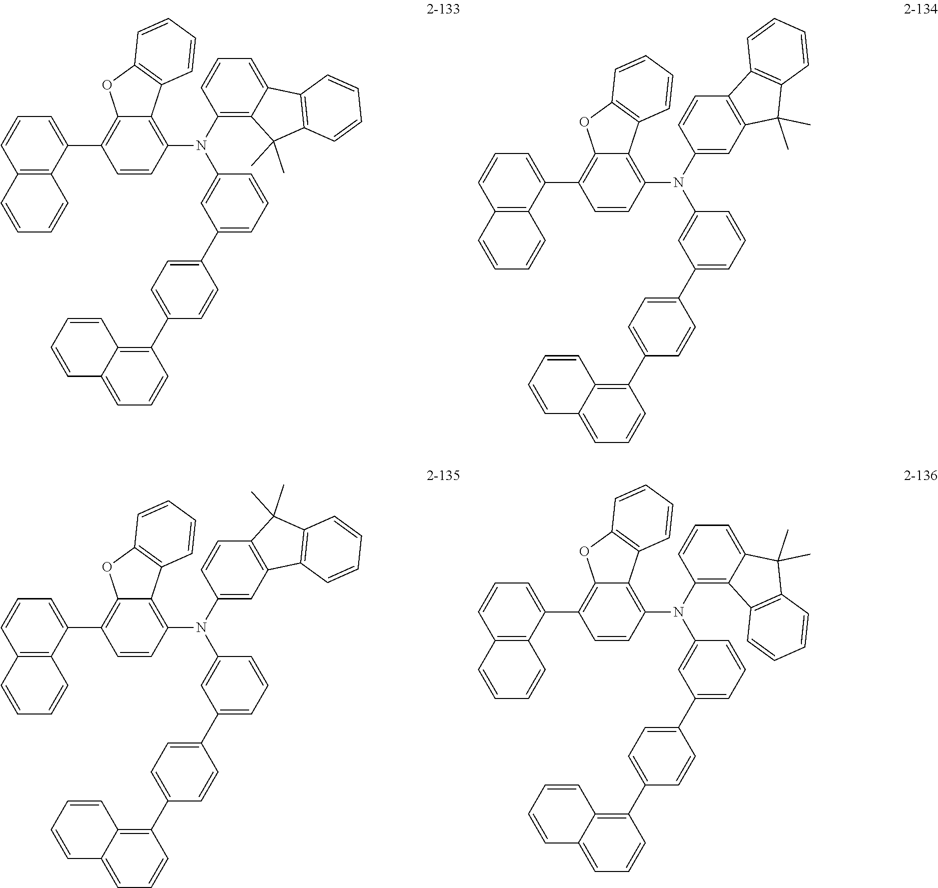

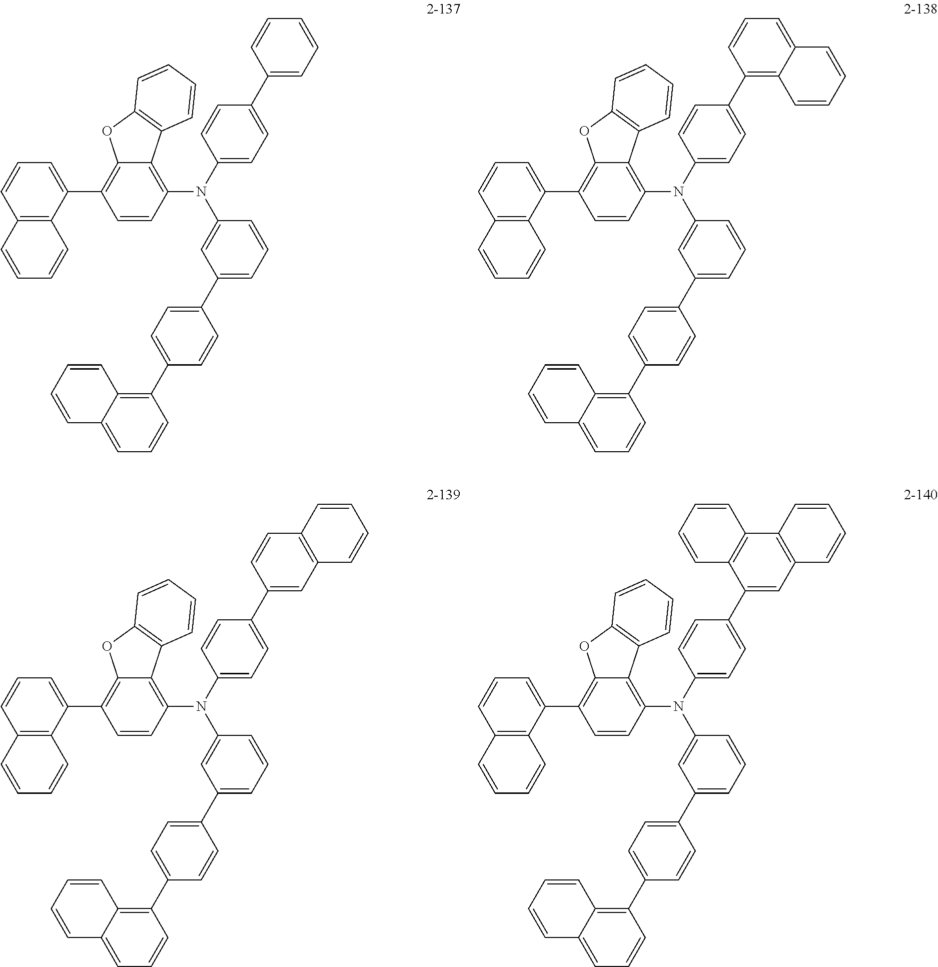 [SMILES: c1ccc(-c2ccc(N(c3cccc(-c4ccc(-c5cccc6ccccc56)cc4)c3)c3ccc(-c4cccc5ccccc45)c4oc5ccccc5c34)cc2)cc1 ; c1cc(-c2ccc(-c3cccc4ccccc34)cc2)cc(N(c2ccc(-c3cccc4ccccc34)cc2)c2ccc(-c3cccc4ccccc34)c3oc4ccccc4c23)c1 ; c1cc(-c2ccc(-c3cccc4ccccc34)cc2)cc(N(c2ccc(-c3ccc4ccccc4c3)cc2)c2ccc(-c3cccc4ccccc34)c3oc4ccccc4c23)c1 ; c1cc(-c2ccc(-c3cccc4ccccc34)cc2)cc(N(c2ccc(-c3cc4ccccc4c4ccccc34)cc2)c2ccc(-c3cccc4ccccc34)c3oc4ccccc4c23)c1]

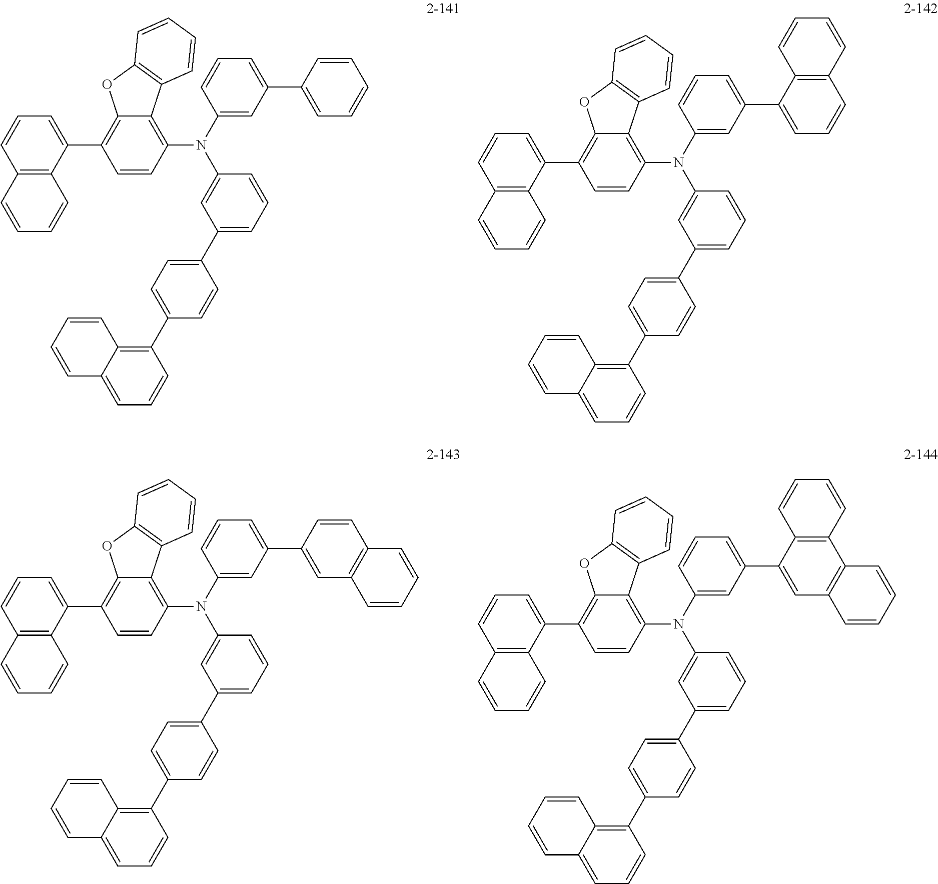 [SMILES: c1ccc(-c2cccc(N(c3cccc(-c4ccc(-c5cccc6ccccc56)cc4)c3)c3ccc(-c4cccc5ccccc45)c4oc5ccccc5c34)c2)cc1 ; c1cc(-c2ccc(-c3cccc4ccccc34)cc2)cc(N(c2cccc(-c3cccc4ccccc34)c2)c2ccc(-c3cccc4ccccc34)c3oc4ccccc4c23)c1 ; c1cc(-c2ccc(-c3cccc4ccccc34)cc2)cc(N(c2cccc(-c3ccc4ccccc4c3)c2)c2ccc(-c3cccc4ccccc34)c3oc4ccccc4c23)c1 ; c1cc(-c2ccc(-c3cccc4ccccc34)cc2)cc(N(c2cccc(-c3cc4ccccc4c4ccccc34)c2)c2ccc(-c3cccc4ccccc34)c3oc4ccccc4c23)c1]

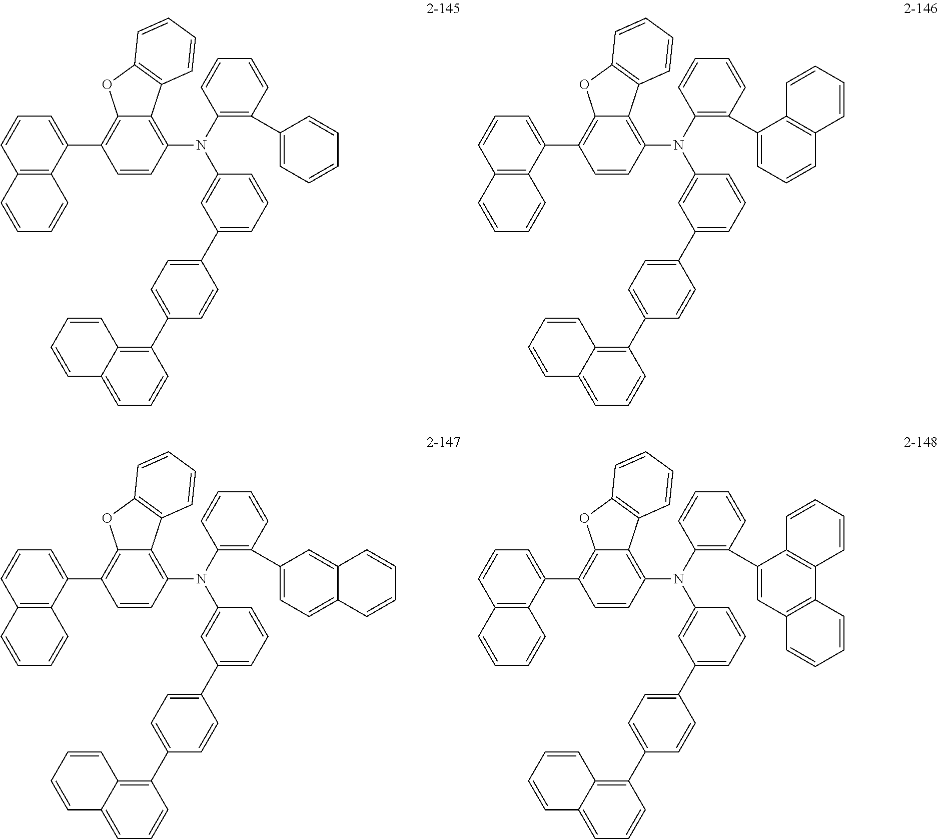 [SMILES: c1ccc(-c2ccccc2N(c2cccc(-c3ccc(-c4cccc5ccccc45)cc3)c2)c2ccc(-c3cccc4ccccc34)c3oc4ccccc4c23)cc1 ; c1cc(-c2ccc(-c3cccc4ccccc34)cc2)cc(N(c2ccccc2-c2cccc3ccccc23)c2ccc(-c3cccc4ccccc34)c3oc4ccccc4c23)c1 ; c1cc(-c2ccc(-c3cccc4ccccc34)cc2)cc(N(c2ccccc2-c2ccc3ccccc3c2)c2ccc(-c3cccc4ccccc34)c3oc4ccccc4c23)c1 ; c1cc(-c2ccc(-c3cccc4ccccc34)cc2)cc(N(c2ccccc2-c2cc3ccccc3c3ccccc23)c2ccc(-c3cccc4ccccc34)c3oc4ccccc4c23)c1]

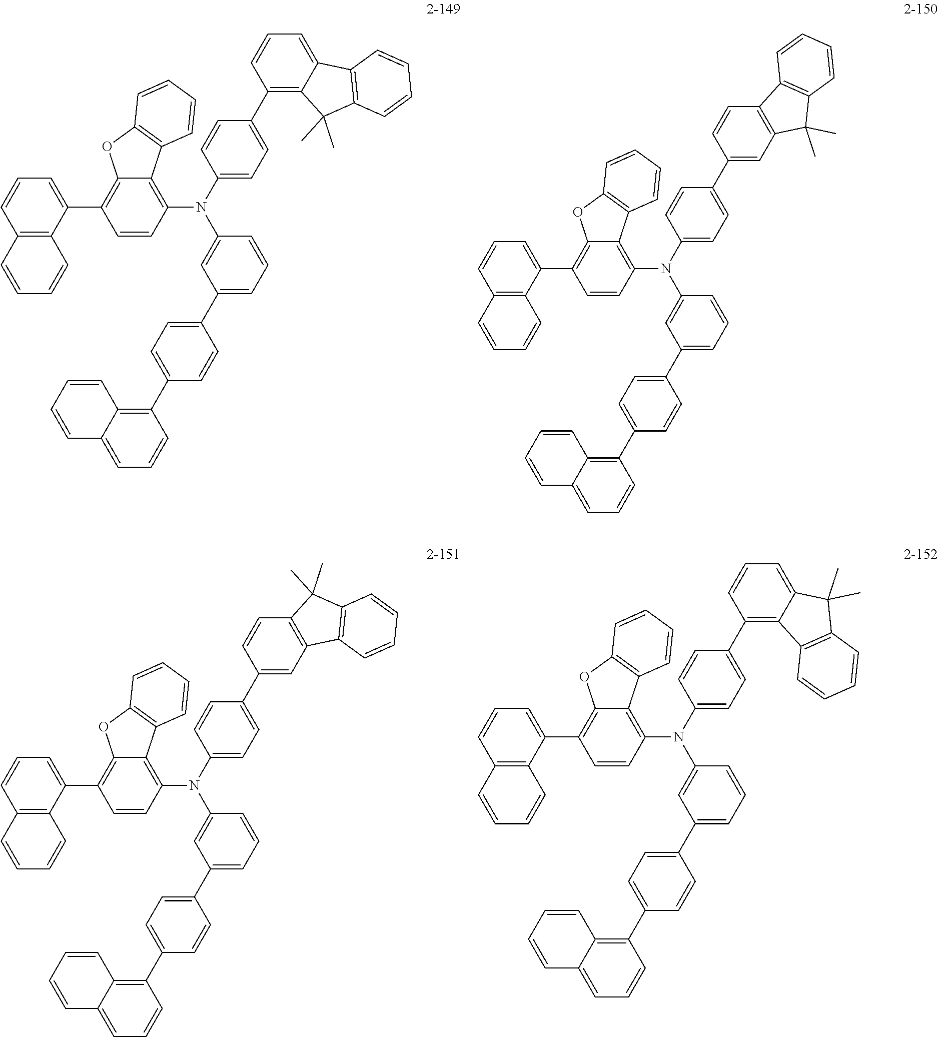 [SMILES: CC1(C)c2ccccc2-c2cccc(-c3ccc(N(c4cccc(-c5ccc(-c6cccc7ccccc67)cc5)c4)c4ccc(-c5cccc6ccccc56)c5oc6ccccc6c45)cc3)c21 ; CC1(C)c2ccccc2-c2ccc(-c3ccc(N(c4cccc(-c5ccc(-c6cccc7ccccc67)cc5)c4)c4ccc(-c5cccc6ccccc56)c5oc6ccccc6c45)cc3)cc21 ; CC1(C)c2ccccc2-c2cc(-c3ccc(N(c4cccc(-c5ccc(-c6cccc7ccccc67)cc5)c4)c4ccc(-c5cccc6ccccc56)c5oc6ccccc6c45)cc3)ccc21 ; CC1(C)c2ccccc2-c2c(-c3ccc(N(c4cccc(-c5ccc(-c6cccc7ccccc67)cc5)c4)c4ccc(-c5cccc6ccccc56)c5oc6ccccc6c45)cc3)cccc21]

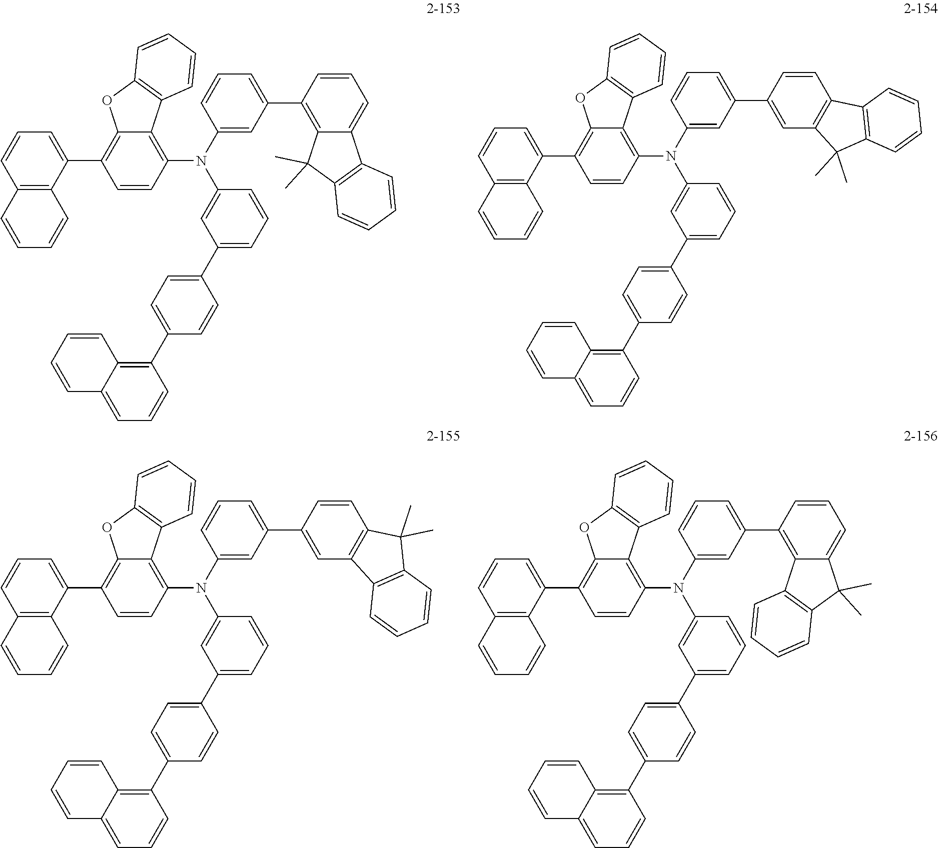

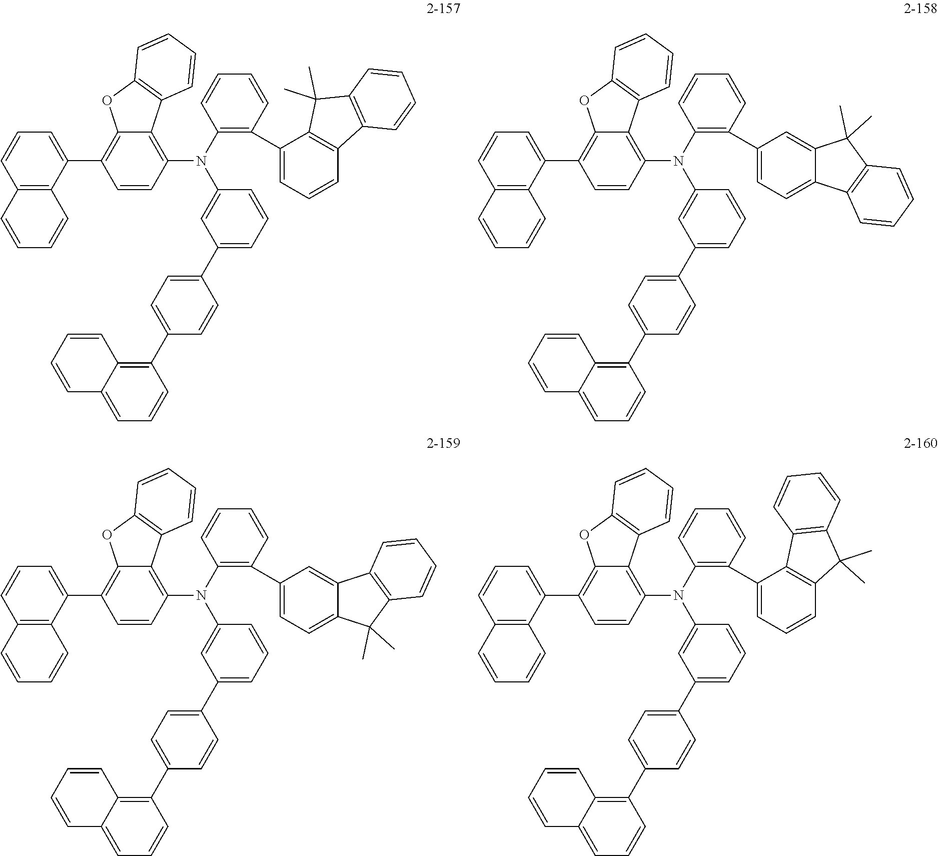 [SMILES: CC1(C)c2ccccc2-c2cccc(-c3ccccc3N(c3cccc(-c4ccc(-c5cccc6ccccc56)cc4)c3)c3ccc(-c4cccc5ccccc45)c4oc5ccccc5c34)c21 ; CC1(C)c2ccccc2-c2ccc(-c3ccccc3N(c3cccc(-c4ccc(-c5cccc6ccccc56)cc4)c3)c3ccc(-c4cccc5ccccc45)c4oc5ccccc5c34)cc21 ; CC1(C)c2ccccc2-c2cc(-c3ccccc3N(c3cccc(-c4ccc(-c5cccc6ccccc56)cc4)c3)c3ccc(-c4cccc5ccccc45)c4oc5ccccc5c34)ccc21 ; CC1(C)c2ccccc2-c2c(-c3ccccc3N(c3cccc(-c4ccc(-c5cccc6ccccc56)cc4)c3)c3ccc(-c4cccc5ccccc45)c4oc5ccccc5c34)cccc21]

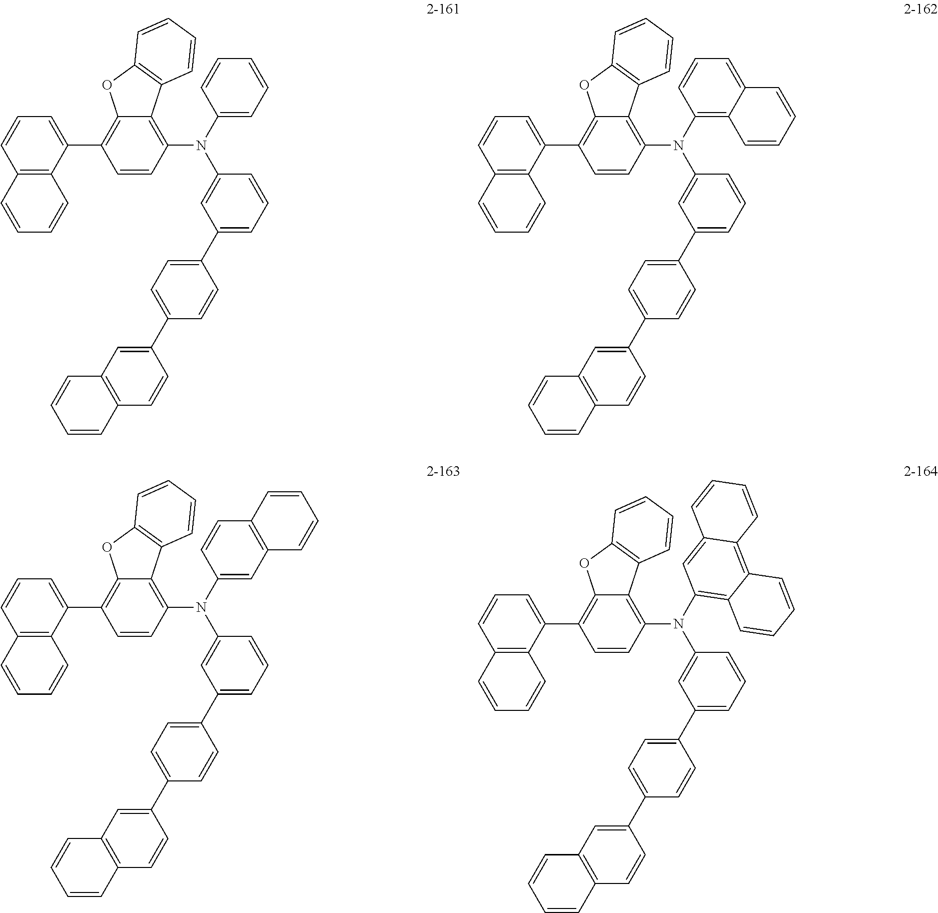 [SMILES: c1ccc(N(c2cccc(-c3ccc(-c4ccc5ccccc5c4)cc3)c2)c2ccc(-c3cccc4ccccc34)c3oc4ccccc4c23)cc1 ; c1cc(-c2ccc(-c3ccc4ccccc4c3)cc2)cc(N(c2cccc3ccccc23)c2ccc(-c3cccc4ccccc34)c3oc4ccccc4c23)c1 ; c1cc(-c2ccc(-c3ccc4ccccc4c3)cc2)cc(N(c2ccc3ccccc3c2)c2ccc(-c3cccc4ccccc34)c3oc4ccccc4c23)c1 ; c1cc(-c2ccc(-c3ccc4ccccc4c3)cc2)cc(N(c2cc3ccccc3c3ccccc23)c2ccc(-c3cccc4ccccc34)c3oc4ccccc4c23)c1]

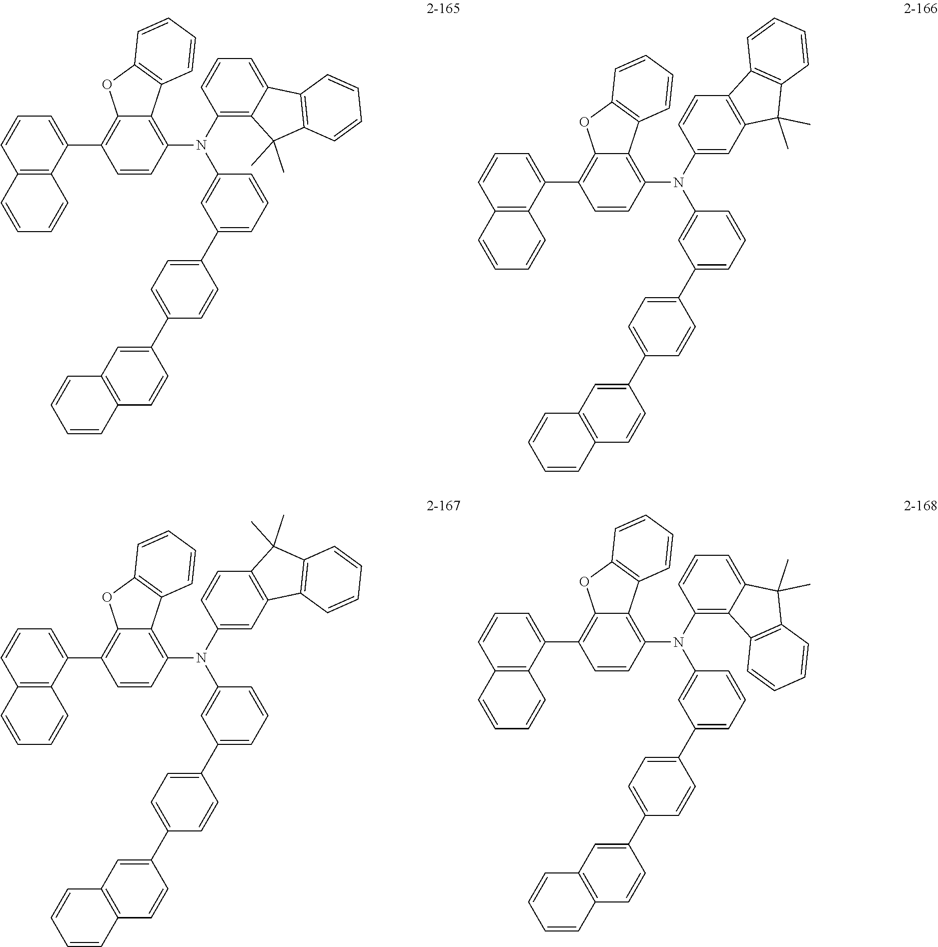 [SMILES: CC1(C)c2ccccc2-c2cccc(N(c3cccc(-c4ccc(-c5ccc6ccccc6c5)cc4)c3)c3ccc(-c4cccc5ccccc45)c4oc5ccccc5c34)c21 ; CC1(C)c2ccccc2-c2ccc(N(c3cccc(-c4ccc(-c5ccc6ccccc6c5)cc4)c3)c3ccc(-c4cccc5ccccc45)c4oc5ccccc5c34)cc21 ; CC1(C)c2ccccc2-c2cc(N(c3cccc(-c4ccc(-c5ccc6ccccc6c5)cc4)c3)c3ccc(-c4cccc5ccccc45)c4oc5ccccc5c34)ccc21 ; CC1(C)c2ccccc2-c2c(N(c3cccc(-c4ccc(-c5ccc6ccccc6c5)cc4)c3)c3ccc(-c4cccc5ccccc45)c4oc5ccccc5c34)cccc21]

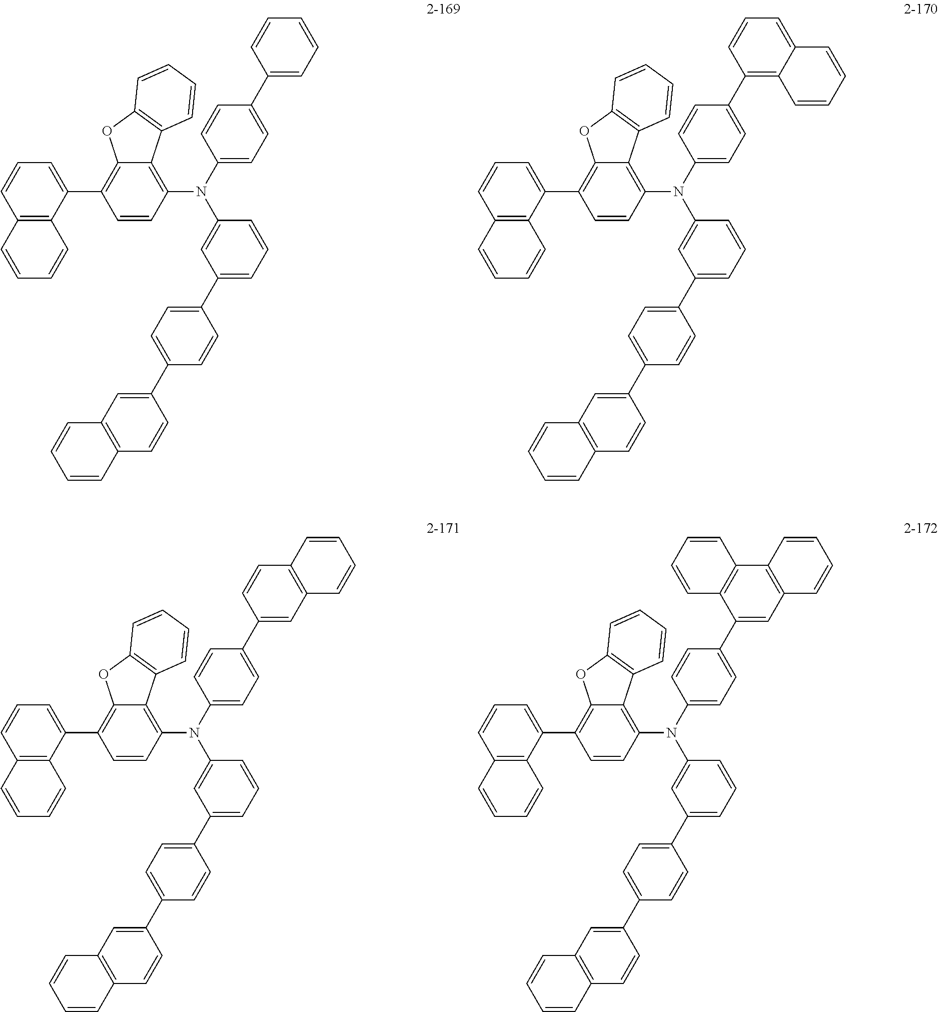 [SMILES: c1ccc(-c2ccc(N(c3cccc(-c4ccc(-c5ccc6ccccc6c5)cc4)c3)c3ccc(-c4cccc5ccccc45)c4oc5ccccc5c34)cc2)cc1 ; c1cc(-c2ccc(-c3ccc4ccccc4c3)cc2)cc(N(c2ccc(-c3cccc4ccccc34)cc2)c2ccc(-c3cccc4ccccc34)c3oc4ccccc4c23)c1 ; c1cc(-c2ccc(-c3ccc4ccccc4c3)cc2)cc(N(c2ccc(-c3ccc4ccccc4c3)cc2)c2ccc(-c3cccc4ccccc34)c3oc4ccccc4c23)c1 ; c1cc(-c2ccc(-c3ccc4ccccc4c3)cc2)cc(N(c2ccc(-c3cc4ccccc4c4ccccc34)cc2)c2ccc(-c3cccc4ccccc34)c3oc4ccccc4c23)c1]

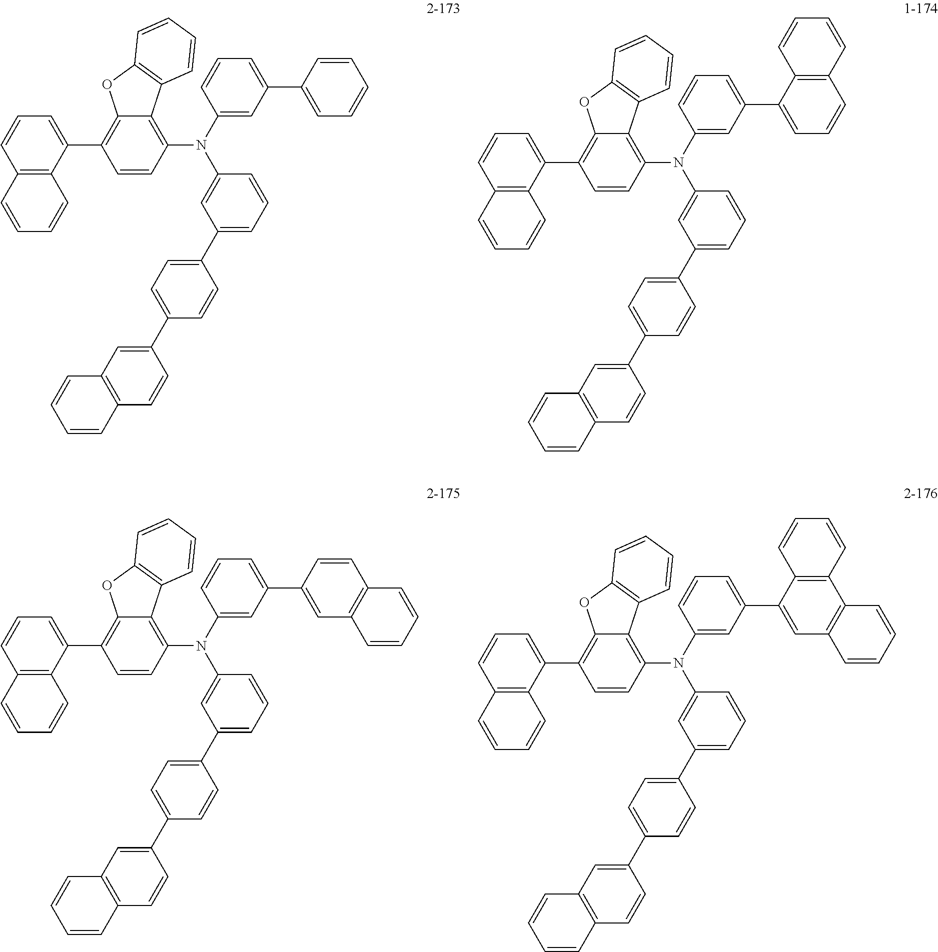 [SMILES: c1ccc(-c2cccc(N(c3cccc(-c4ccc(-c5ccc6ccccc6c5)cc4)c3)c3ccc(-c4cccc5ccccc45)c4oc5ccccc5c34)c2)cc1 ; c1cc(-c2ccc(-c3ccc4ccccc4c3)cc2)cc(N(c2cccc(-c3cccc4ccccc34)c2)c2ccc(-c3cccc4ccccc34)c3oc4ccccc4c23)c1 ; c1cc(-c2ccc(-c3ccc4ccccc4c3)cc2)cc(N(c2cccc(-c3ccc4ccccc4c3)c2)c2ccc(-c3cccc4ccccc34)c3oc4ccccc4c23)c1 ; c1cc(-c2ccc(-c3ccc4ccccc4c3)cc2)cc(N(c2cccc(-c3cc4ccccc4c4ccccc34)c2)c2ccc(-c3cccc4ccccc34)c3oc4ccccc4c23)c1]

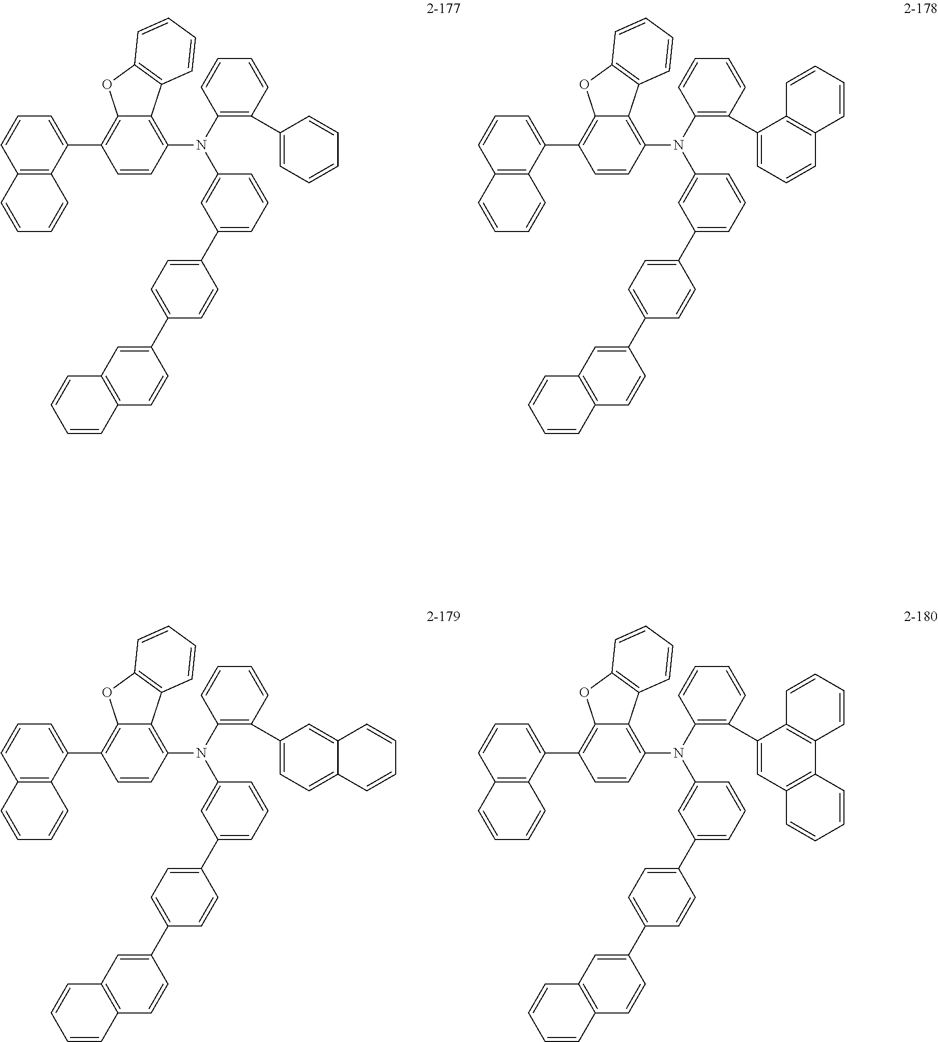 [SMILES: c1ccc(-c2ccccc2N(c2cccc(-c3ccc(-c4ccc5ccccc5c4)cc3)c2)c2ccc(-c3cccc4ccccc34)c3oc4ccccc4c23)cc1 ; c1cc(-c2ccc(-c3ccc4ccccc4c3)cc2)cc(N(c2ccccc2-c2cccc3ccccc23)c2ccc(-c3cccc4ccccc34)c3oc4ccccc4c23)c1 ; c1cc(-c2ccc(-c3ccc4ccccc4c3)cc2)cc(N(c2ccccc2-c2ccc3ccccc3c2)c2ccc(-c3cccc4ccccc34)c3oc4ccccc4c23)c1 ; c1cc(-c2ccc(-c3ccc4ccccc4c3)cc2)cc(N(c2ccccc2-c2cc3ccccc3c3ccccc23)c2ccc(-c3cccc4ccccc34)c3oc4ccccc4c23)c1]

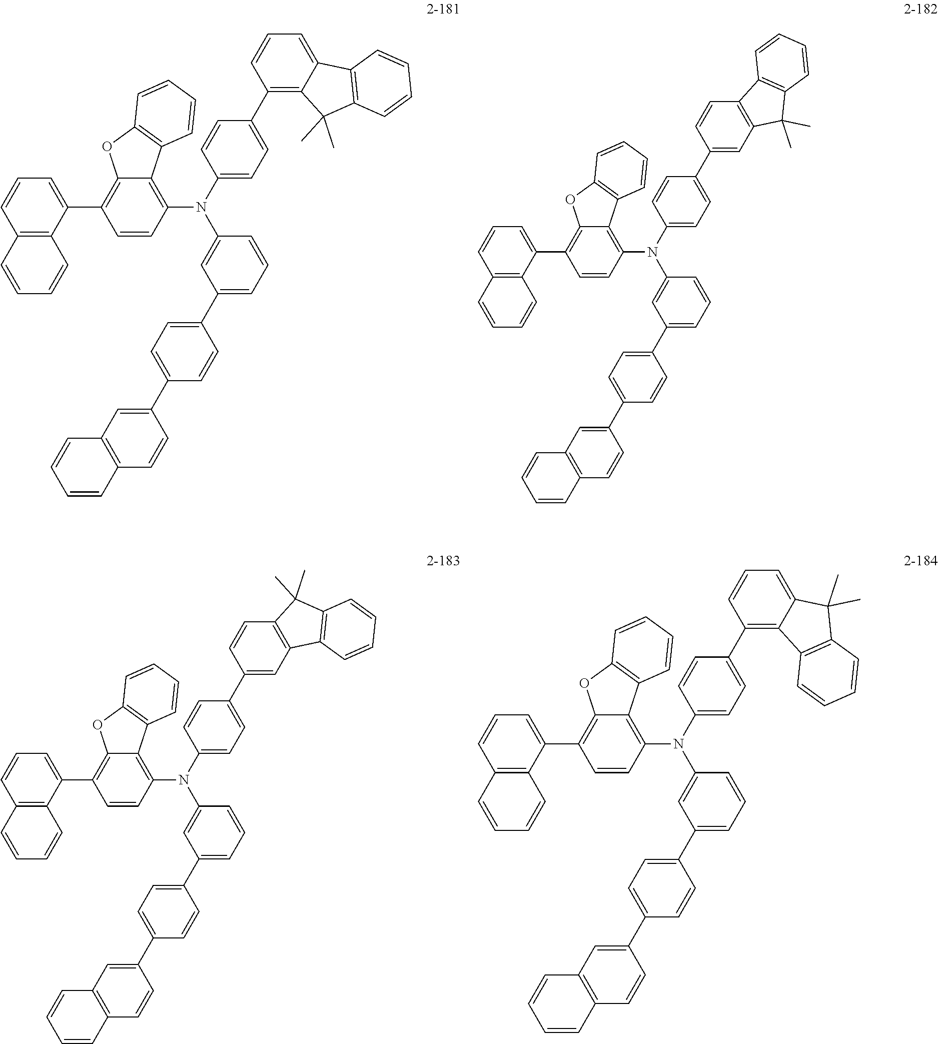 [SMILES: CC1(C)c2ccccc2-c2cccc(-c3ccc(N(c4cccc(-c5ccc(-c6ccc7ccccc7c6)cc5)c4)c4ccc(-c5cccc6ccccc56)c5oc6ccccc6c45)cc3)c21 ; CC1(C)c2ccccc2-c2ccc(-c3ccc(N(c4cccc(-c5ccc(-c6ccc7ccccc7c6)cc5)c4)c4ccc(-c5cccc6ccccc56)c5oc6ccccc6c45)cc3)cc21 ; CC1(C)c2ccccc2-c2cc(-c3ccc(N(c4cccc(-c5ccc(-c6ccc7ccccc7c6)cc5)c4)c4ccc(-c5cccc6ccccc56)c5oc6ccccc6c45)cc3)ccc21 ; CC1(C)c2ccccc2-c2c(-c3ccc(N(c4cccc(-c5ccc(-c6ccc7ccccc7c6)cc5)c4)c4ccc(-c5cccc6ccccc56)c5oc6ccccc6c45)cc3)cccc21]

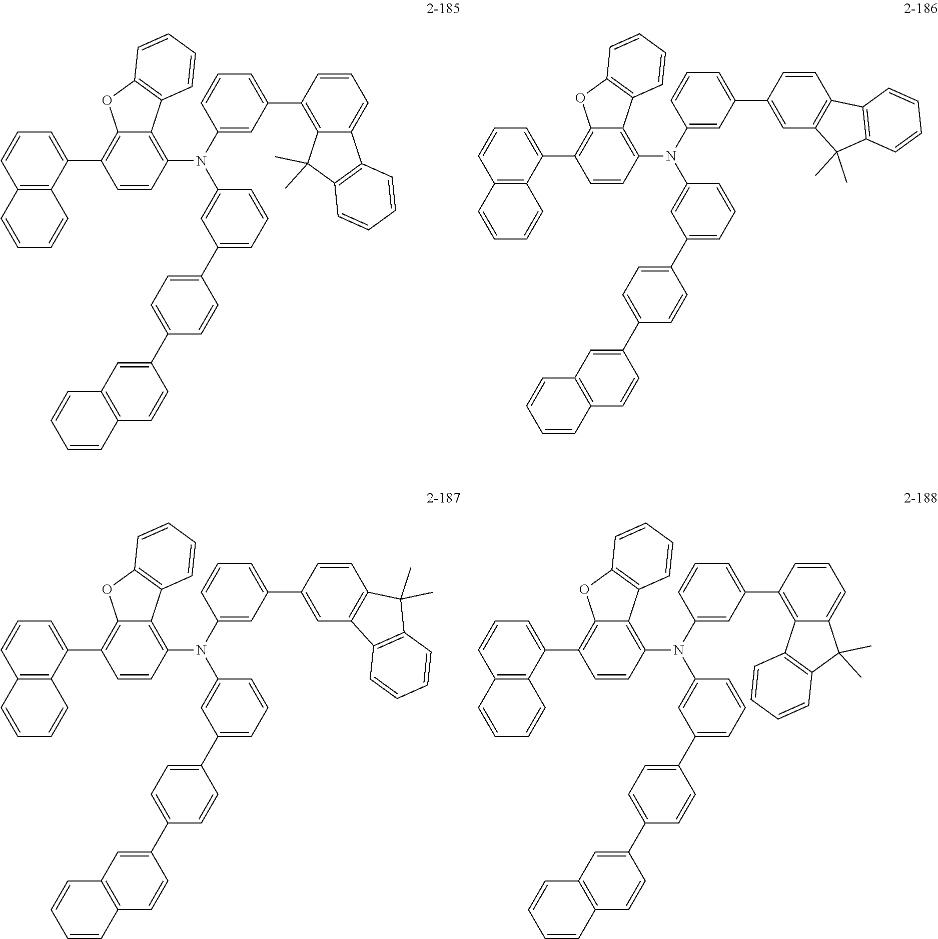 [SMILES: CC1(C)c2ccccc2-c2cccc(-c3cccc(N(c4cccc(-c5ccc(-c6ccc7ccccc7c6)cc5)c4)c4ccc(-c5cccc6ccccc56)c5oc6ccccc6c45)c3)c21 ; CC1(C)c2ccccc2-c2ccc(-c3cccc(N(c4cccc(-c5ccc(-c6ccc7ccccc7c6)cc5)c4)c4ccc(-c5cccc6ccccc56)c5oc6ccccc6c45)c3)cc21 ; CC1(C)c2ccccc2-c2cc(-c3cccc(N(c4cccc(-c5ccc(-c6ccc7ccccc7c6)cc5)c4)c4ccc(-c5cccc6ccccc56)c5oc6ccccc6c45)c3)ccc21 ; CC1(C)c2ccccc2-c2c(-c3cccc(N(c4cccc(-c5ccc(-c6ccc7ccccc7c6)cc5)c4)c4ccc(-c5cccc6ccccc56)c5oc6ccccc6c45)c3)cccc21]

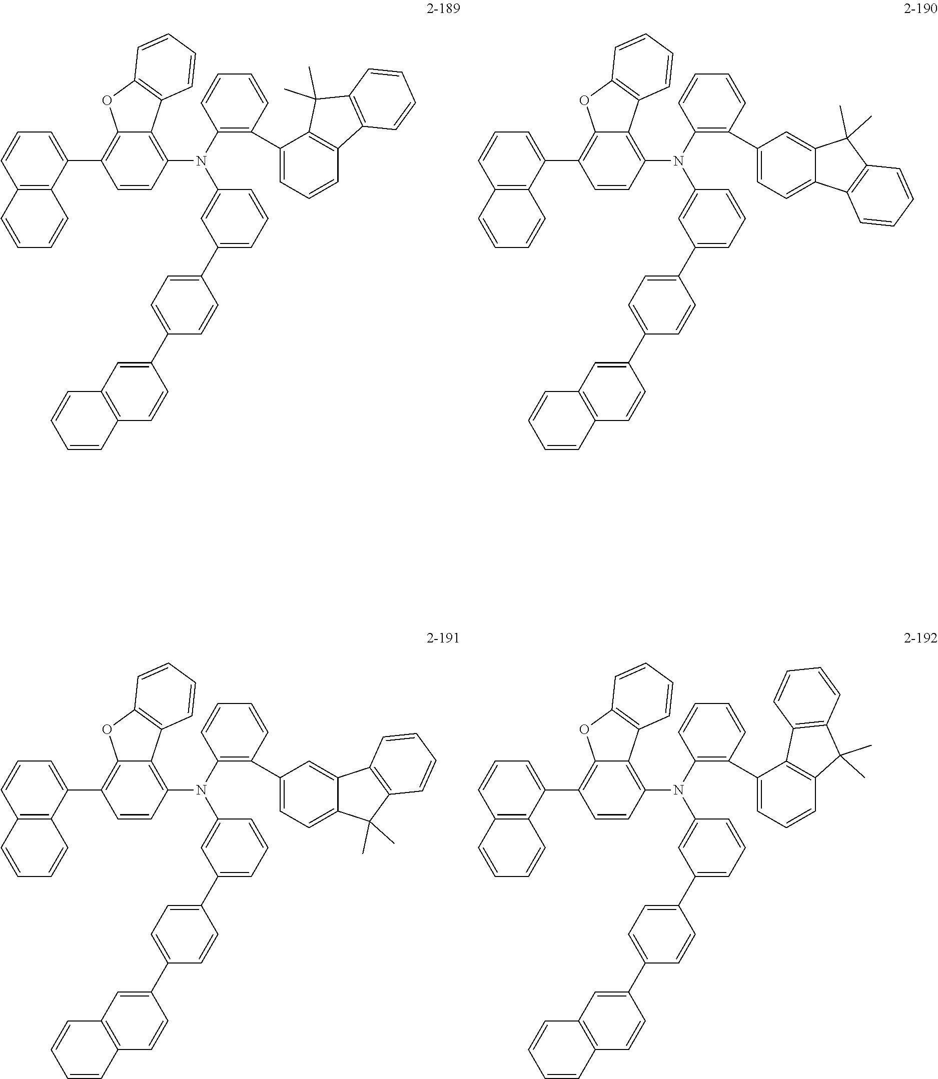 [SMILES: CC1(C)c2ccccc2-c2cccc(-c3ccccc3N(c3cccc(-c4ccc(-c5ccc6ccccc6c5)cc4)c3)c3ccc(-c4cccc5ccccc45)c4oc5ccccc5c34)c21 ; CC1(C)c2ccccc2-c2ccc(-c3ccccc3N(c3cccc(-c4ccc(-c5ccc6ccccc6c5)cc4)c3)c3ccc(-c4cccc5ccccc45)c4oc5ccccc5c34)cc21 ; CC1(C)c2ccccc2-c2cc(-c3ccccc3N(c3cccc(-c4ccc(-c5ccc6ccccc6c5)cc4)c3)c3ccc(-c4cccc5ccccc45)c4oc5ccccc5c34)ccc21 ; CC1(C)c2ccccc2-c2c(-c3ccccc3N(c3cccc(-c4ccc(-c5ccc6ccccc6c5)cc4)c3)c3ccc(-c4cccc5ccccc45)c4oc5ccccc5c34)cccc21]

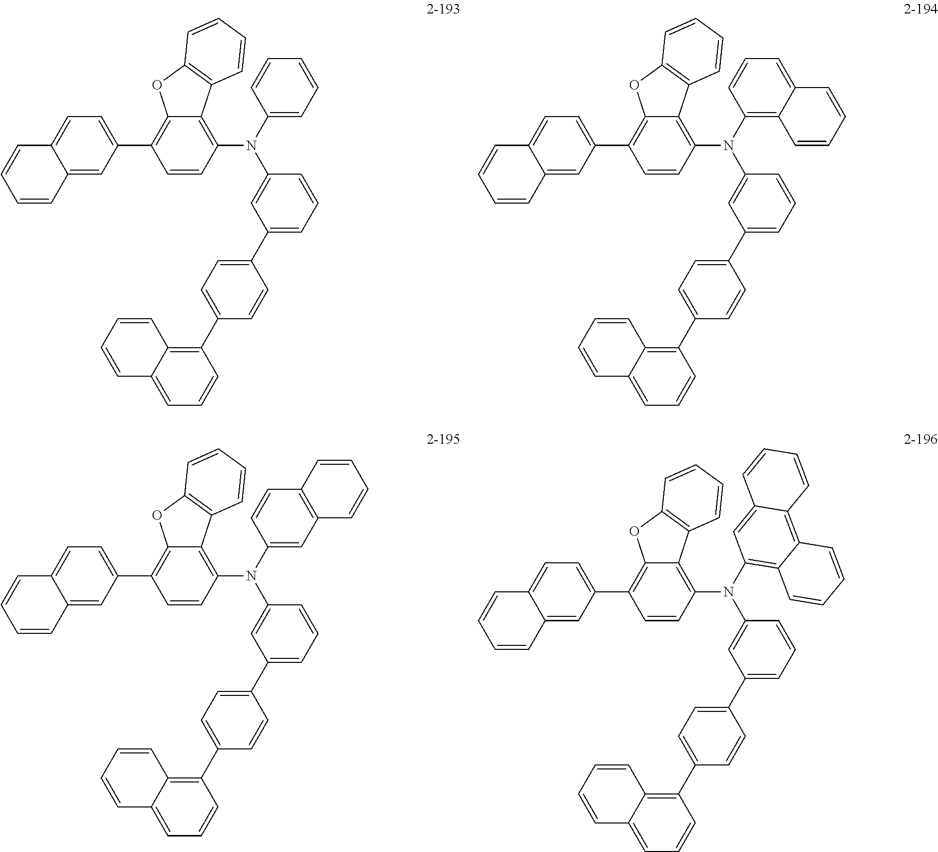 [SMILES: c1ccc(N(c2cccc(-c3ccc(-c4cccc5ccccc45)cc3)c2)c2ccc(-c3ccc4ccccc4c3)c3oc4ccccc4c23)cc1 ; c1cc(-c2ccc(-c3cccc4ccccc34)cc2)cc(N(c2cccc3ccccc23)c2ccc(-c3ccc4ccccc4c3)c3oc4ccccc4c23)c1 ; c1cc(-c2ccc(-c3cccc4ccccc34)cc2)cc(N(c2ccc3ccccc3c2)c2ccc(-c3ccc4ccccc4c3)c3oc4ccccc4c23)c1 ; c1cc(-c2ccc(-c3cccc4ccccc34)cc2)cc(N(c2cc3ccccc3c3ccccc23)c2ccc(-c3ccc4ccccc4c3)c3oc4ccccc4c23)c1]

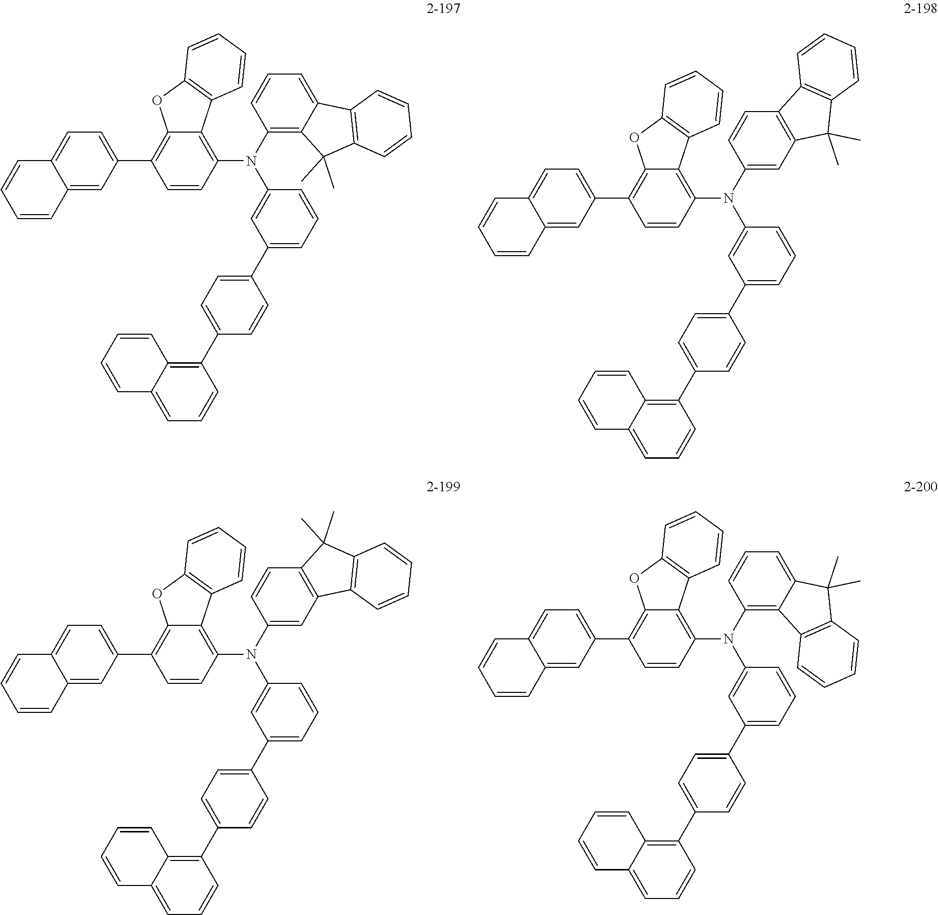 [SMILES: CC1(C)c2ccccc2-c2cccc(N(c3cccc(-c4ccc(-c5cccc6ccccc56)cc4)c3)c3ccc(-c4ccc5ccccc5c4)c4oc5ccccc5c34)c21 ; CC1(C)c2ccccc2-c2ccc(N(c3cccc(-c4ccc(-c5cccc6ccccc56)cc4)c3)c3ccc(-c4ccc5ccccc5c4)c4oc5ccccc5c34)cc21 ; CC1(C)c2ccccc2-c2cc(N(c3cccc(-c4ccc(-c5cccc6ccccc56)cc4)c3)c3ccc(-c4ccc5ccccc5c4)c4oc5ccccc5c34)ccc21 ; CC1(C)c2ccccc2-c2c(N(c3cccc(-c4ccc(-c5cccc6ccccc56)cc4)c3)c3ccc(-c4ccc5ccccc5c4)c4oc5ccccc5c34)cccc21]

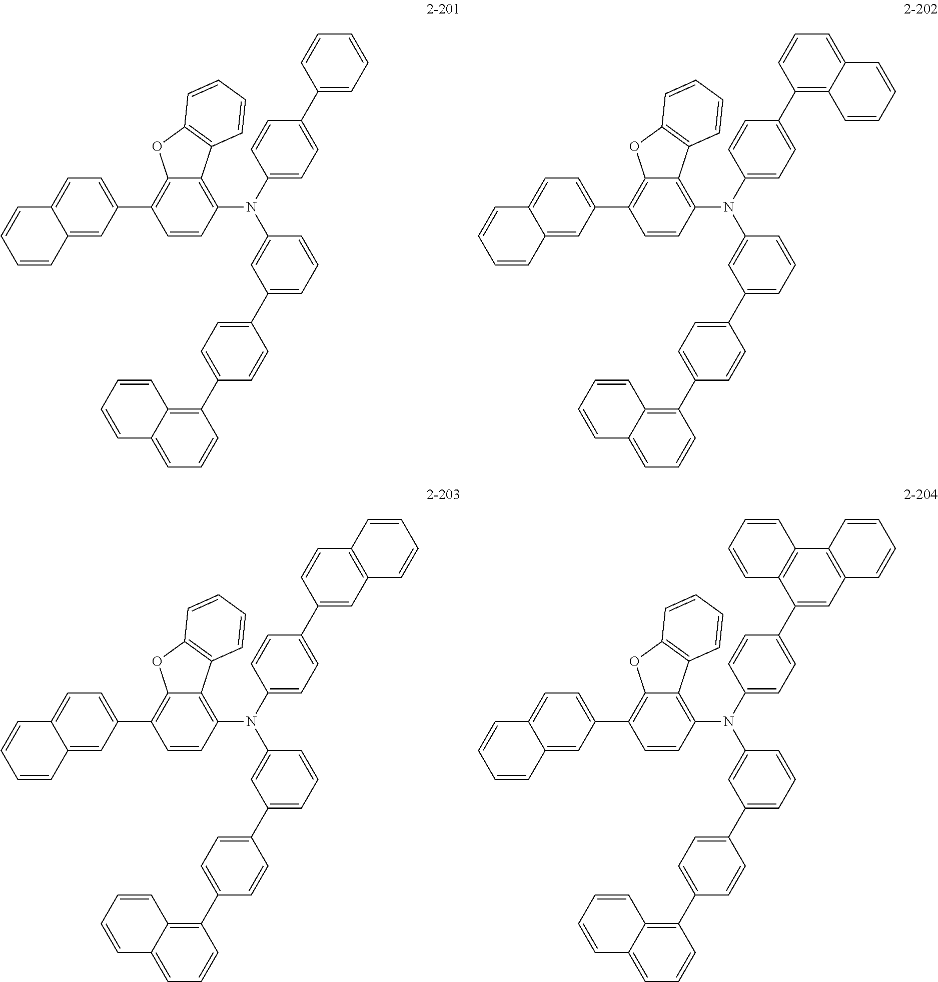 [SMILES: c1ccc(-c2ccc(N(c3cccc(-c4ccc(-c5cccc6ccccc56)cc4)c3)c3ccc(-c4ccc5ccccc5c4)c4oc5ccccc5c34)cc2)cc1 ; c1cc(-c2ccc(-c3cccc4ccccc34)cc2)cc(N(c2ccc(-c3cccc4ccccc34)cc2)c2ccc(-c3ccc4ccccc4c3)c3oc4ccccc4c23)c1 ; c1cc(-c2ccc(-c3cccc4ccccc34)cc2)cc(N(c2ccc(-c3ccc4ccccc4c3)cc2)c2ccc(-c3ccc4ccccc4c3)c3oc4ccccc4c23)c1 ; c1cc(-c2ccc(-c3cccc4ccccc34)cc2)cc(N(c2ccc(-c3cc4ccccc4c4ccccc34)cc2)c2ccc(-c3ccc4ccccc4c3)c3oc4ccccc4c23)c1]

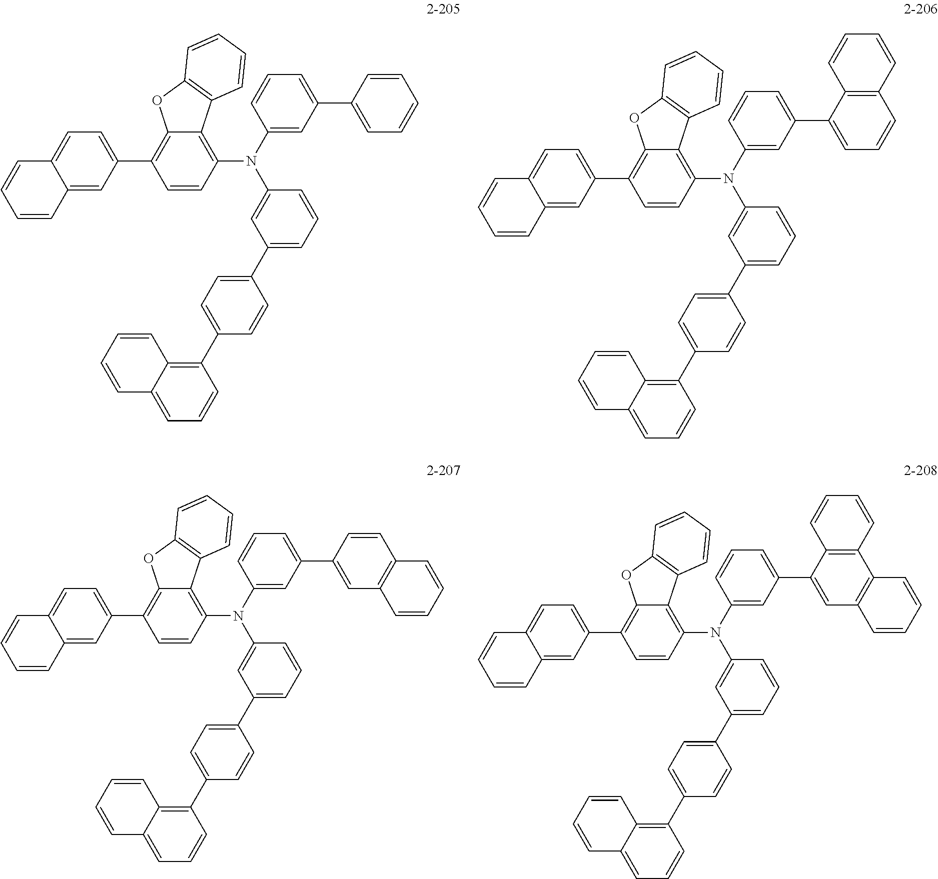 [SMILES: c1ccc(-c2cccc(N(c3cccc(-c4ccc(-c5cccc6ccccc56)cc4)c3)c3ccc(-c4ccc5ccccc5c4)c4oc5ccccc5c34)c2)cc1 ; c1cc(-c2ccc(-c3cccc4ccccc34)cc2)cc(N(c2cccc(-c3cccc4ccccc34)c2)c2ccc(-c3ccc4ccccc4c3)c3oc4ccccc4c23)c1 ; c1cc(-c2ccc(-c3cccc4ccccc34)cc2)cc(N(c2cccc(-c3ccc4ccccc4c3)c2)c2ccc(-c3ccc4ccccc4c3)c3oc4ccccc4c23)c1 ; c1cc(-c2ccc(-c3cccc4ccccc34)cc2)cc(N(c2cccc(-c3cc4ccccc4c4ccccc34)c2)c2ccc(-c3ccc4ccccc4c3)c3oc4ccccc4c23)c1]

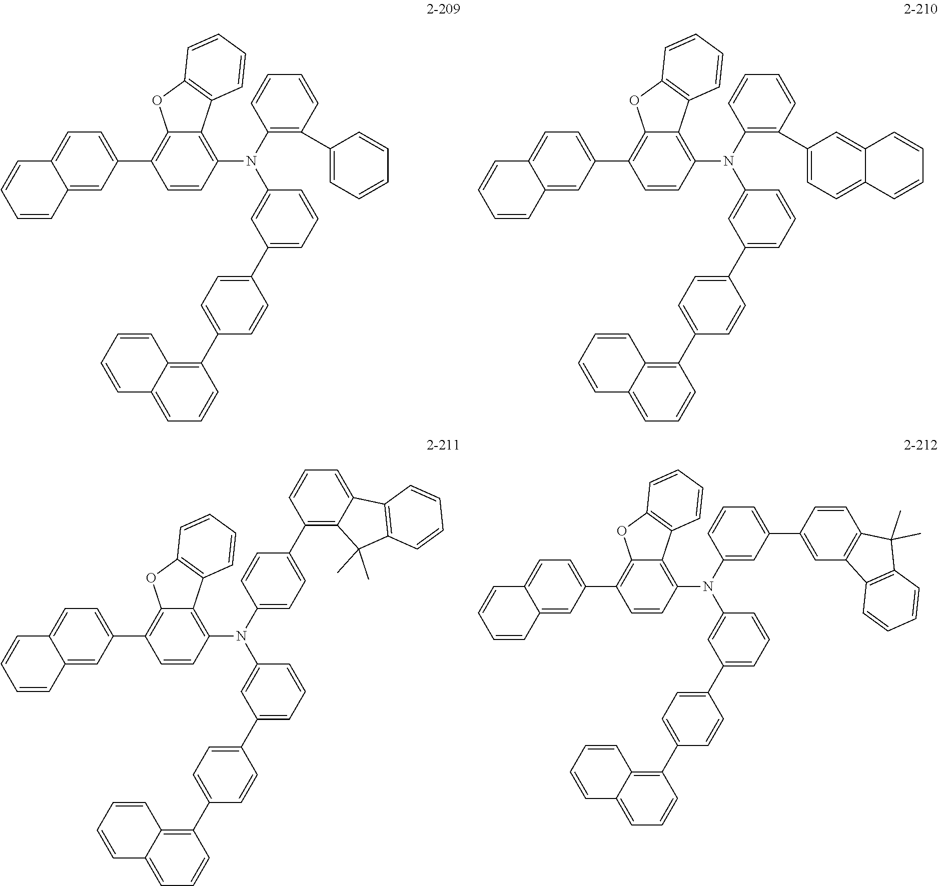 [SMILES: c1ccc(-c2ccccc2N(c2cccc(-c3ccc(-c4cccc5ccccc45)cc3)c2)c2ccc(-c3ccc4ccccc4c3)c3oc4ccccc4c23)cc1 ; c1cc(-c2ccc(-c3cccc4ccccc34)cc2)cc(N(c2ccccc2-c2ccc3ccccc3c2)c2ccc(-c3ccc4ccccc4c3)c3oc4ccccc4c23)c1 ; CC1(C)c2ccccc2-c2cccc(-c3ccc(N(c4cccc(-c5ccc(-c6cccc7ccccc67)cc5)c4)c4ccc(-c5ccc6ccccc6c5)c5oc6ccccc6c45)cc3)c21 ; CC1(C)c2ccccc2-c2cc(-c3cccc(N(c4cccc(-c5ccc(-c6cccc7ccccc67)cc5)c4)c4ccc(-c5ccc6ccccc6c5)c5oc6ccccc6c45)c3)ccc21]

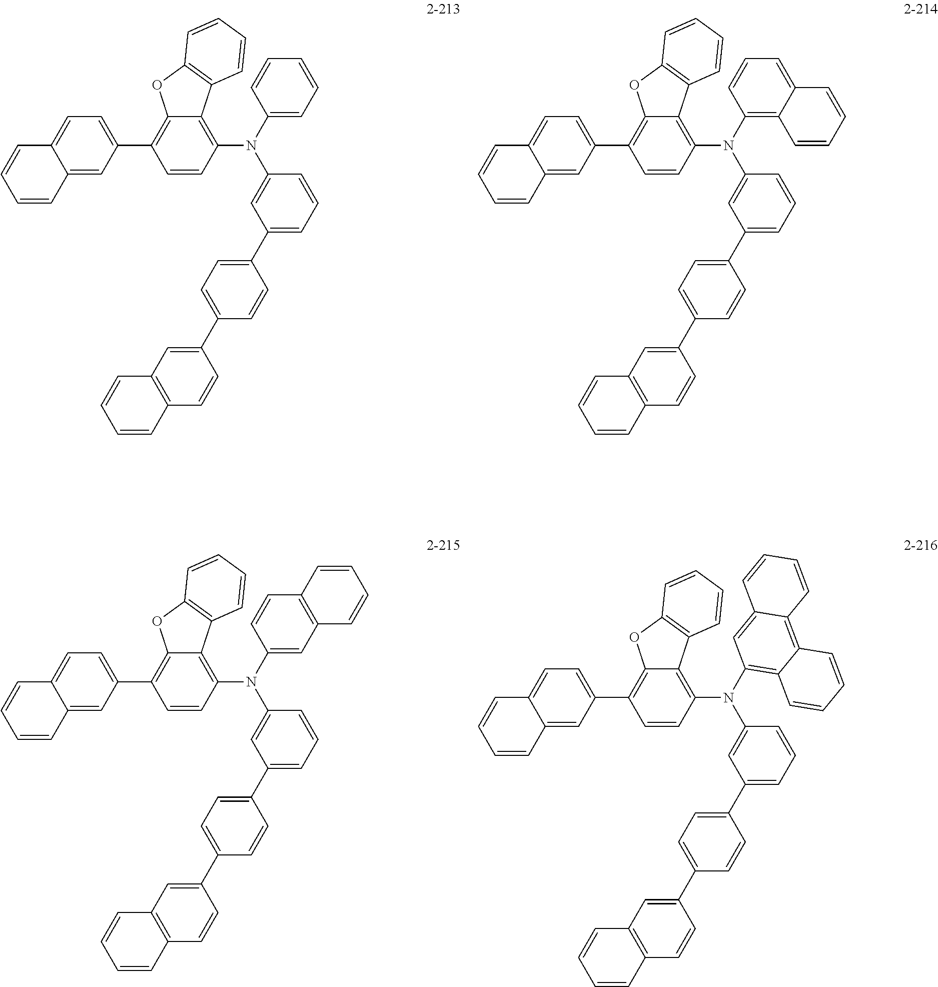 [SMILES: c1ccc(N(c2cccc(-c3ccc(-c4ccc5ccccc5c4)cc3)c2)c2ccc(-c3ccc4ccccc4c3)c3oc4ccccc4c23)cc1 ; c1cc(-c2ccc(-c3ccc4ccccc4c3)cc2)cc(N(c2cccc3ccccc23)c2ccc(-c3ccc4ccccc4c3)c3oc4ccccc4c23)c1 ; c1cc(-c2ccc(-c3ccc4ccccc4c3)cc2)cc(N(c2ccc3ccccc3c2)c2ccc(-c3ccc4ccccc4c3)c3oc4ccccc4c23)c1 ; c1cc(-c2ccc(-c3ccc4ccccc4c3)cc2)cc(N(c2cc3ccccc3c3ccccc23)c2ccc(-c3ccc4ccccc4c3)c3oc4ccccc4c23)c1]

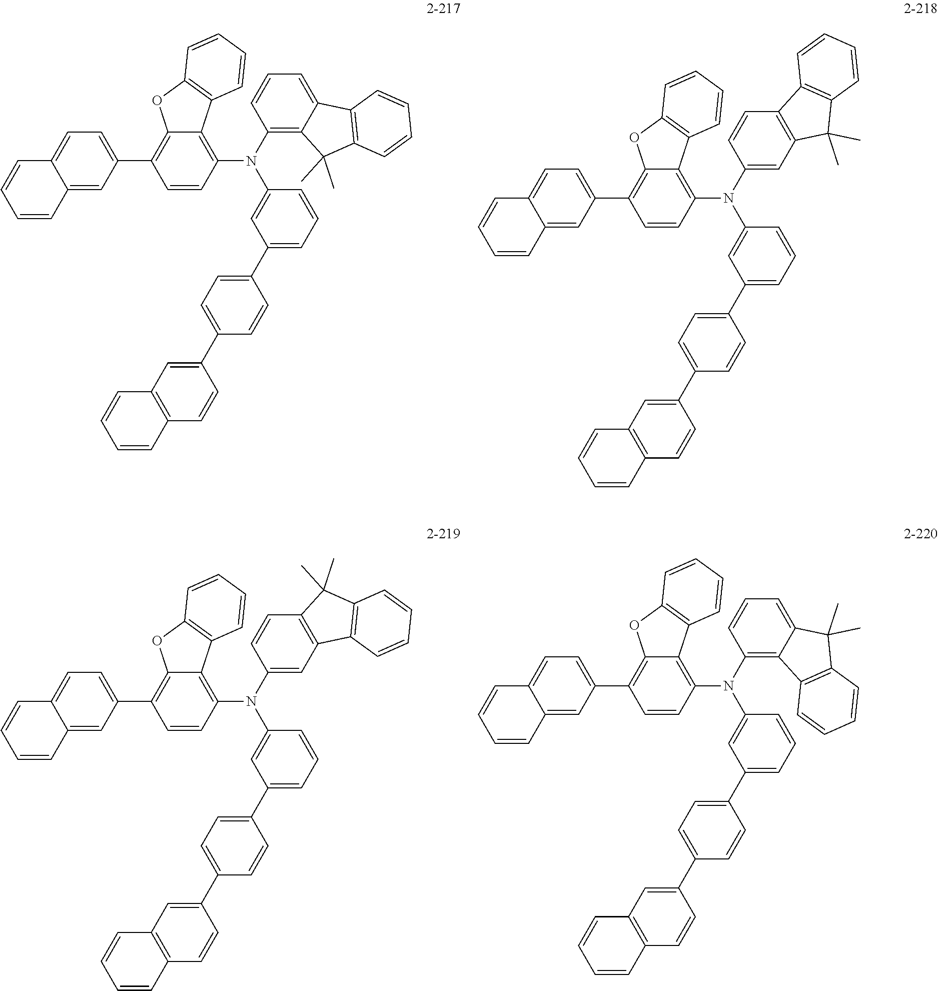 [SMILES: CC1(C)c2ccccc2-c2cccc(N(c3cccc(-c4ccc(-c5ccc6ccccc6c5)cc4)c3)c3ccc(-c4ccc5ccccc5c4)c4oc5ccccc5c34)c21 ; CC1(C)c2ccccc2-c2ccc(N(c3cccc(-c4ccc(-c5ccc6ccccc6c5)cc4)c3)c3ccc(-c4ccc5ccccc5c4)c4oc5ccccc5c34)cc21 ; CC1(C)c2ccccc2-c2cc(N(c3cccc(-c4ccc(-c5ccc6ccccc6c5)cc4)c3)c3ccc(-c4ccc5ccccc5c4)c4oc5ccccc5c34)ccc21 ; CC1(C)c2ccccc2-c2c(N(c3cccc(-c4ccc(-c5ccc6ccccc6c5)cc4)c3)c3ccc(-c4ccc5ccccc5c4)c4oc5ccccc5c34)cccc21]

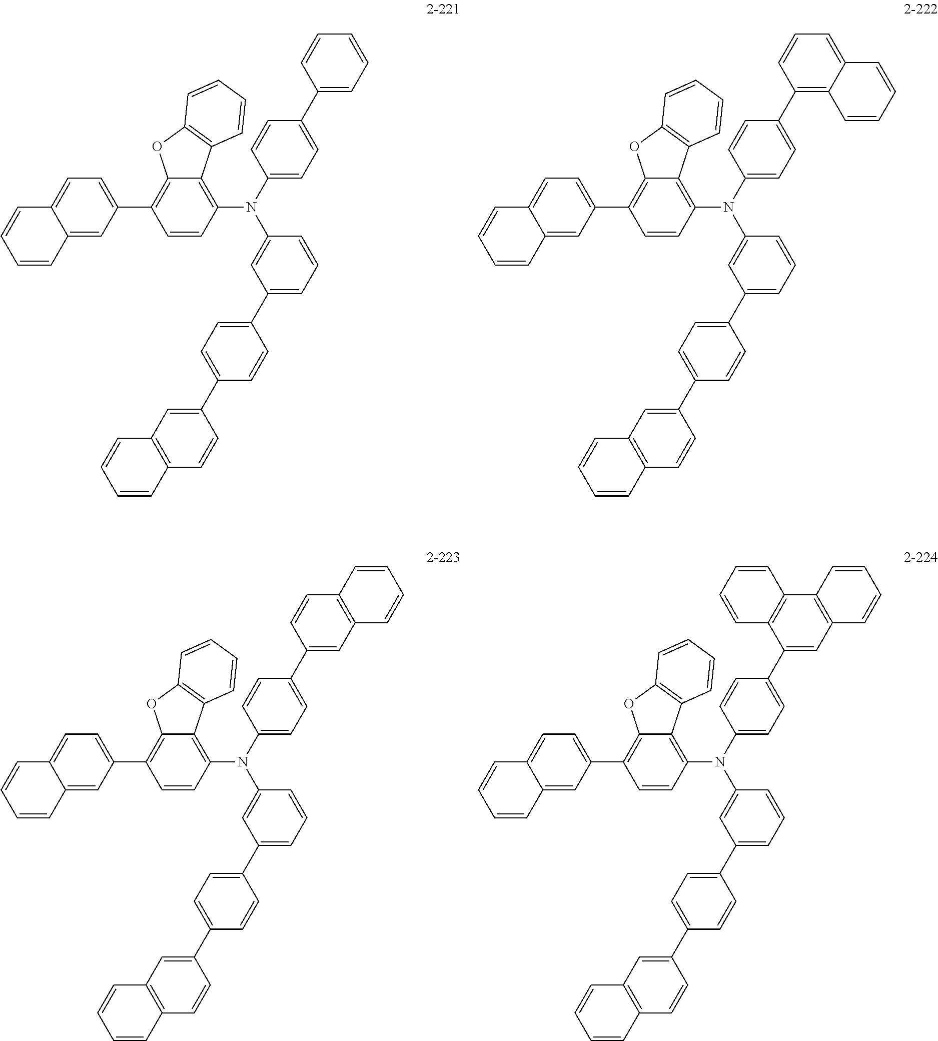 [SMILES: c1ccc(-c2ccc(N(c3cccc(-c4ccc(-c5ccc6ccccc6c5)cc4)c3)c3ccc(-c4ccc5ccccc5c4)c4oc5ccccc5c34)cc2)cc1 ; c1cc(-c2ccc(-c3ccc4ccccc4c3)cc2)cc(N(c2ccc(-c3cccc4ccccc34)cc2)c2ccc(-c3ccc4ccccc4c3)c3oc4ccccc4c23)c1 ; c1cc(-c2ccc(-c3ccc4ccccc4c3)cc2)cc(N(c2ccc(-c3ccc4ccccc4c3)cc2)c2ccc(-c3ccc4ccccc4c3)c3oc4ccccc4c23)c1 ; c1cc(-c2ccc(-c3ccc4ccccc4c3)cc2)cc(N(c2ccc(-c3cc4ccccc4c4ccccc34)cc2)c2ccc(-c3ccc4ccccc4c3)c3oc4ccccc4c23)c1]

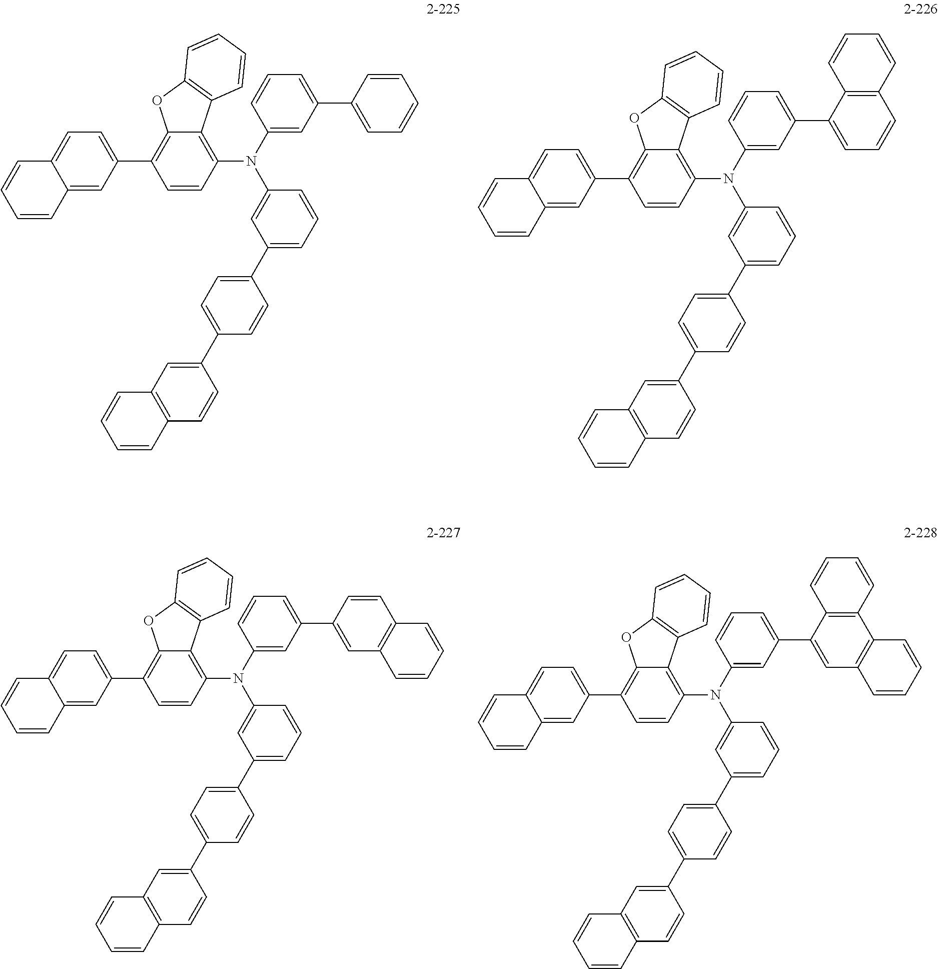 [SMILES: c1ccc(-c2cccc(N(c3cccc(-c4ccc(-c5ccc6ccccc6c5)cc4)c3)c3ccc(-c4ccc5ccccc5c4)c4oc5ccccc5c34)c2)cc1 ; c1cc(-c2ccc(-c3ccc4ccccc4c3)cc2)cc(N(c2cccc(-c3cccc4ccccc34)c2)c2ccc(-c3ccc4ccccc4c3)c3oc4ccccc4c23)c1 ; c1cc(-c2ccc(-c3ccc4ccccc4c3)cc2)cc(N(c2cccc(-c3ccc4ccccc4c3)c2)c2ccc(-c3ccc4ccccc4c3)c3oc4ccccc4c23)c1 ; c1cc(-c2ccc(-c3ccc4ccccc4c3)cc2)cc(N(c2cccc(-c3cc4ccccc4c4ccccc34)c2)c2ccc(-c3ccc4ccccc4c3)c3oc4ccccc4c23)c1]

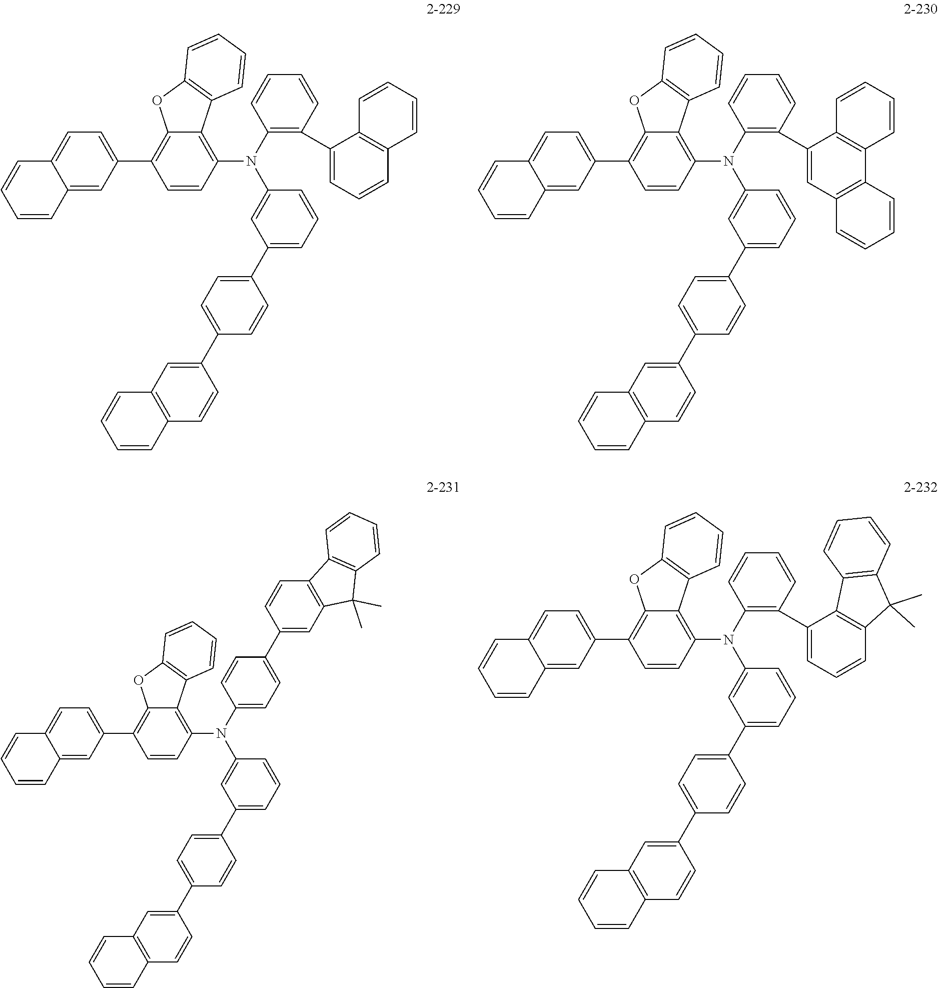 [SMILES: c1cc(-c2ccc(-c3ccc4ccccc4c3)cc2)cc(N(c2ccccc2-c2cccc3ccccc23)c2ccc(-c3ccc4ccccc4c3)c3oc4ccccc4c23)c1 ; c1cc(-c2ccc(-c3ccc4ccccc4c3)cc2)cc(N(c2ccccc2-c2cc3ccccc3c3ccccc23)c2ccc(-c3ccc4ccccc4c3)c3oc4ccccc4c23)c1 ; CC1(C)c2ccccc2-c2ccc(-c3ccc(N(c4cccc(-c5ccc(-c6ccc7ccccc7c6)cc5)c4)c4ccc(-c5ccc6ccccc6c5)c5oc6ccccc6c45)cc3)cc21 ; CC1(C)c2ccccc2-c2c(-c3ccccc3N(c3cccc(-c4ccc(-c5ccc6ccccc6c5)cc4)c3)c3ccc(-c4ccc5ccccc5c4)c4oc5ccccc5c34)cccc21]

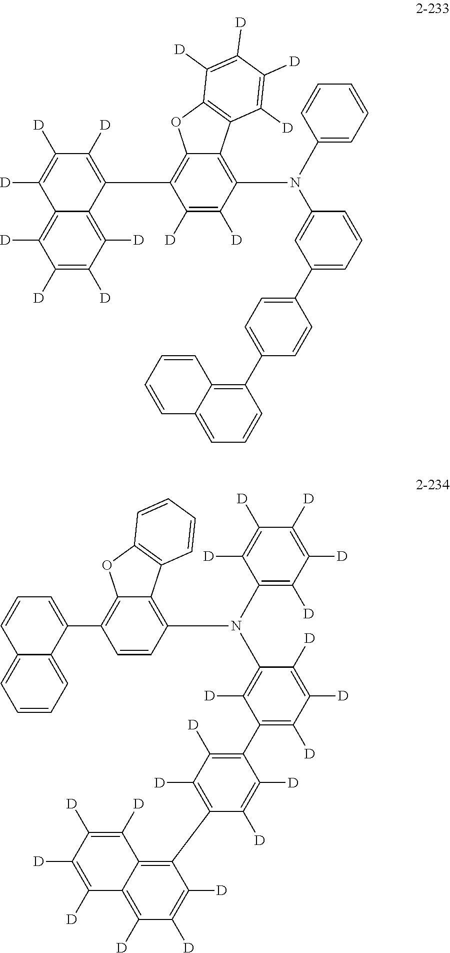 [SMILES: [2H]c1c([2H])c([2H])c2c(oc3c(-c4c([2H])c([2H])c([2H])c5c([2H])c([2H])c([2H])c([2H])c45)c([2H])c([2H])c(N(c4ccccc4)c4cccc(-c5ccc(-c6cccc7ccccc67)cc5)c4)c32)c1[2H] ; [2H]c1c([2H])c([2H])c(N(c2c([2H])c([2H])c([2H])c(-c3c([2H])c([2H])c(-c4c([2H])c([2H])c([2H])c5c([2H])c([2H])c([2H])c([2H])c45)c([2H])c3[2H])c2[2H])c2ccc(-c3cccc4ccccc34)c3oc4ccccc4c23)c([2H])c1[2H]]

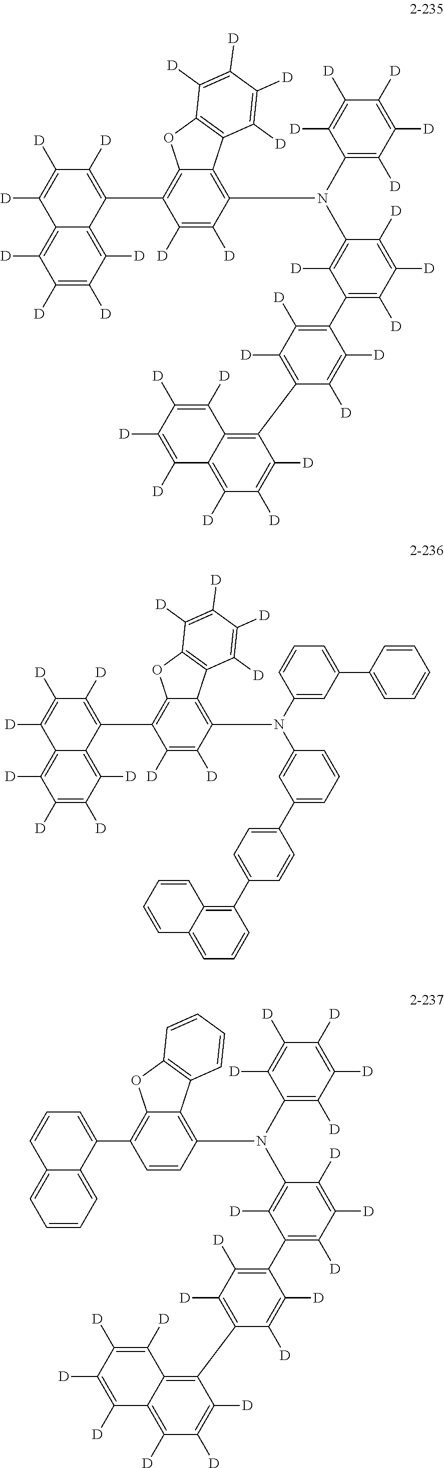

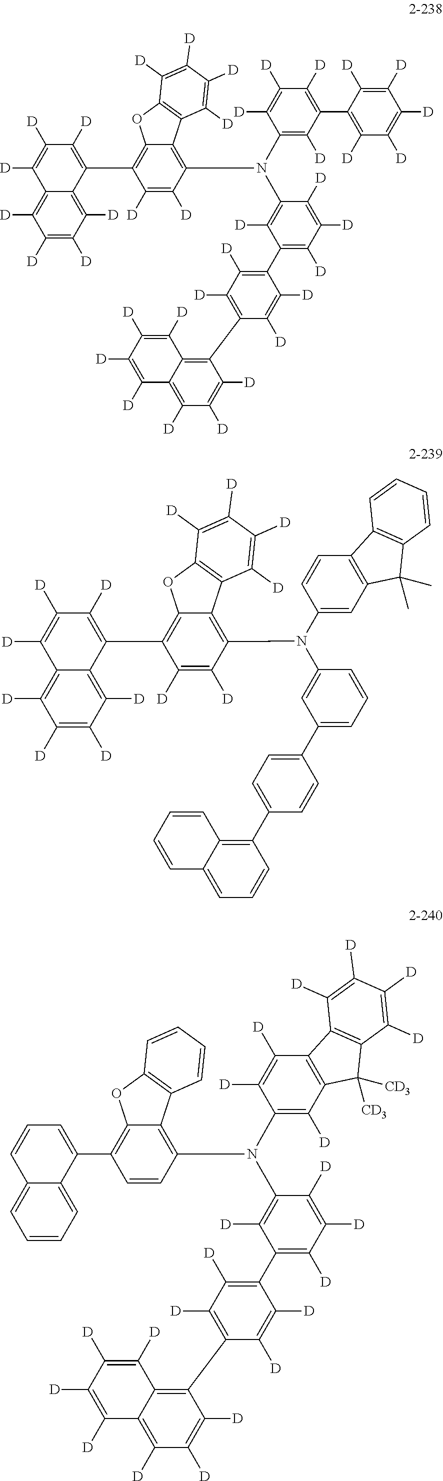

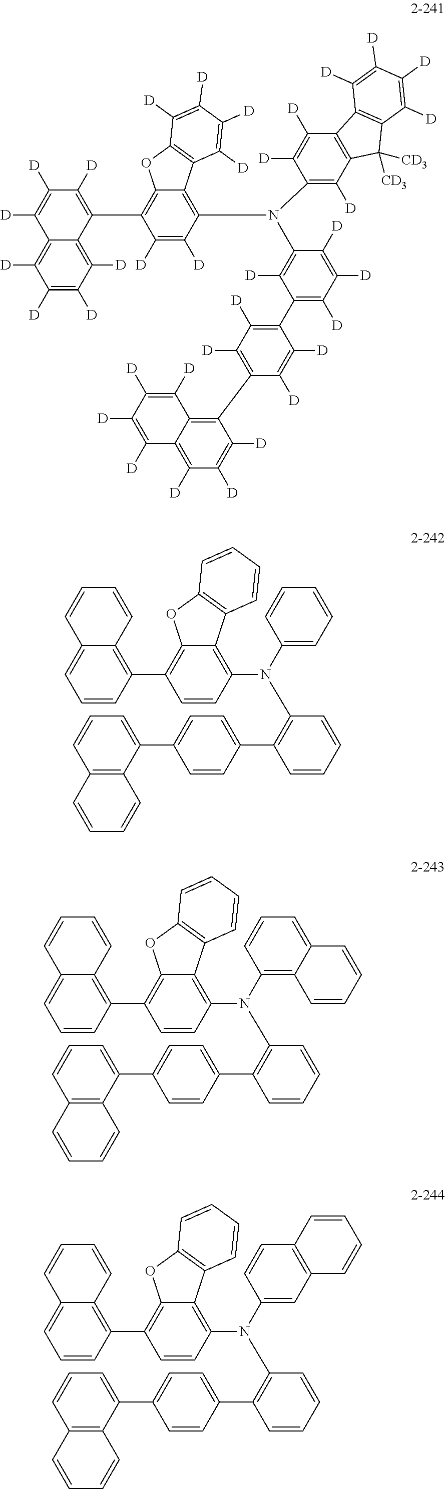

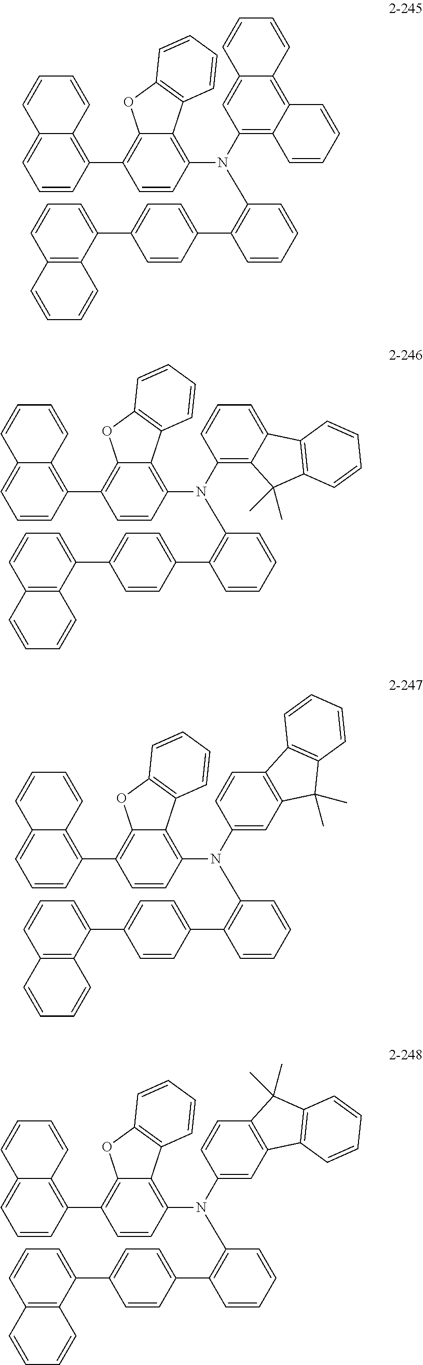 [SMILES: c1ccc(N(c2cc3ccccc3c3ccccc23)c2ccc(-c3cccc4ccccc34)c3oc4ccccc4c23)c(-c2ccc(-c3cccc4ccccc34)cc2)c1 ; CC1(C)c2ccccc2-c2cccc(N(c3ccccc3-c3ccc(-c4cccc5ccccc45)cc3)c3ccc(-c4cccc5ccccc45)c4oc5ccccc5c34)c21 ; CC1(C)c2ccccc2-c2ccc(N(c3ccccc3-c3ccc(-c4cccc5ccccc45)cc3)c3ccc(-c4cccc5ccccc45)c4oc5ccccc5c34)cc21 ; CC1(C)c2ccccc2-c2cc(N(c3ccccc3-c3ccc(-c4cccc5ccccc45)cc3)c3ccc(-c4cccc5ccccc45)c4oc5ccccc5c34)ccc21]

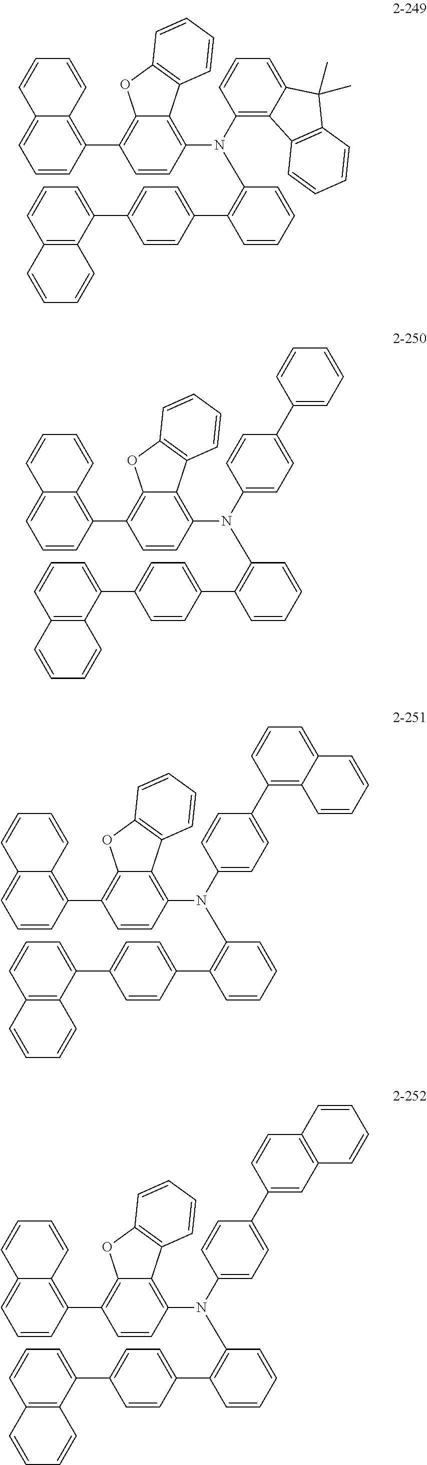 [SMILES: CC1(C)c2ccccc2-c2c(N(c3ccccc3-c3ccc(-c4cccc5ccccc45)cc3)c3ccc(-c4cccc5ccccc45)c4oc5ccccc5c34)cccc21 ; c1ccc(-c2ccc(N(c3ccccc3-c3ccc(-c4cccc5ccccc45)cc3)c3ccc(-c4cccc5ccccc45)c4oc5ccccc5c34)cc2)cc1 ; c1ccc(N(c2ccc(-c3cccc4ccccc34)cc2)c2ccc(-c3cccc4ccccc34)c3oc4ccccc4c23)c(-c2ccc(-c3cccc4ccccc34)cc2)c1 ; c1ccc(N(c2ccc(-c3ccc4ccccc4c3)cc2)c2ccc(-c3cccc4ccccc34)c3oc4ccccc4c23)c(-c2ccc(-c3cccc4ccccc34)cc2)c1]

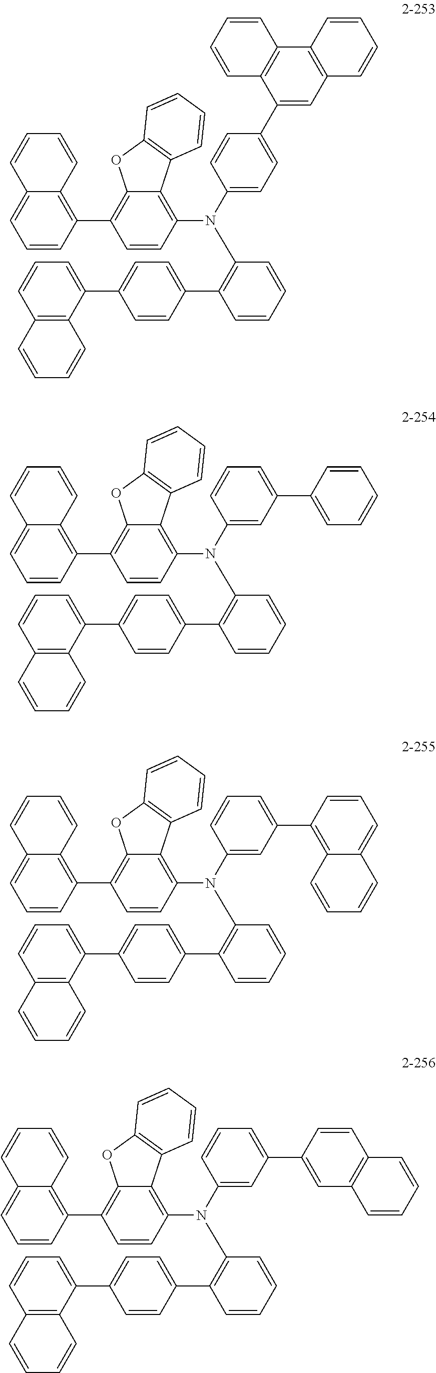 [SMILES: c1ccc(N(c2ccc(-c3cc4ccccc4c4ccccc34)cc2)c2ccc(-c3cccc4ccccc34)c3oc4ccccc4c23)c(-c2ccc(-c3cccc4ccccc34)cc2)c1 ; c1ccc(-c2cccc(N(c3ccccc3-c3ccc(-c4cccc5ccccc45)cc3)c3ccc(-c4cccc5ccccc45)c4oc5ccccc5c34)c2)cc1 ; c1cc(-c2cccc3ccccc23)cc(N(c2ccccc2-c2ccc(-c3cccc4ccccc34)cc2)c2ccc(-c3cccc4ccccc34)c3oc4ccccc4c23)c1 ; c1cc(-c2ccc3ccccc3c2)cc(N(c2ccccc2-c2ccc(-c3cccc4ccccc34)cc2)c2ccc(-c3cccc4ccccc34)c3oc4ccccc4c23)c1]

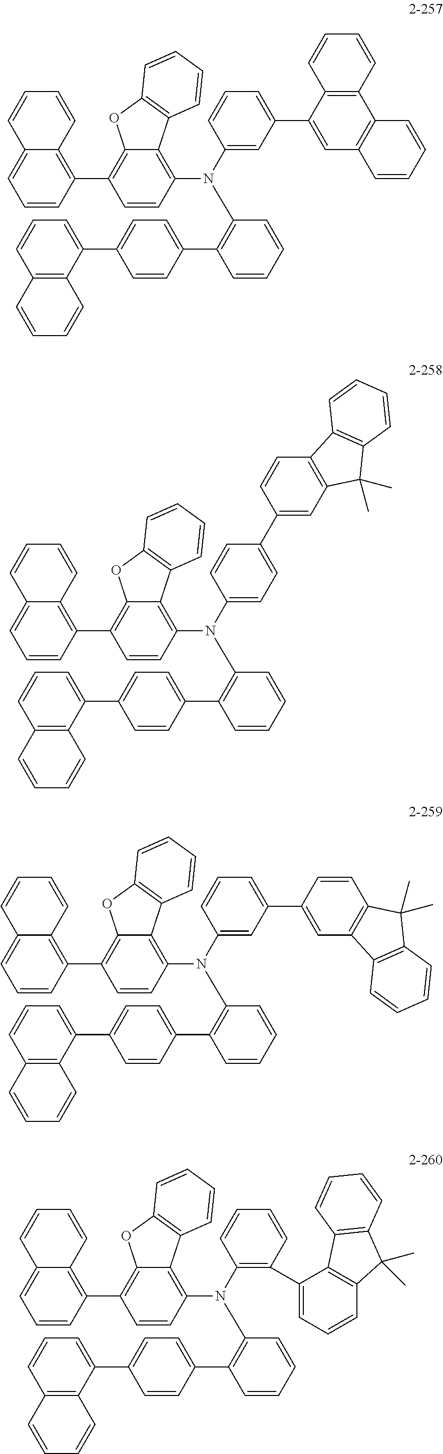 [SMILES: c1cc(-c2cc3ccccc3c3ccccc23)cc(N(c2ccccc2-c2ccc(-c3cccc4ccccc34)cc2)c2ccc(-c3cccc4ccccc34)c3oc4ccccc4c23)c1 ; CC1(C)c2ccccc2-c2ccc(-c3ccc(N(c4ccccc4-c4ccc(-c5cccc6ccccc56)cc4)c4ccc(-c5cccc6ccccc56)c5oc6ccccc6c45)cc3)cc21 ; CC1(C)c2ccccc2-c2cc(-c3cccc(N(c4ccccc4-c4ccc(-c5cccc6ccccc56)cc4)c4ccc(-c5cccc6ccccc56)c5oc6ccccc6c45)c3)ccc21 ; CC1(C)c2ccccc2-c2c(-c3ccccc3N(c3ccccc3-c3ccc(-c4cccc5ccccc45)cc3)c3ccc(-c4cccc5ccccc45)c4oc5ccccc5c34)cccc21]

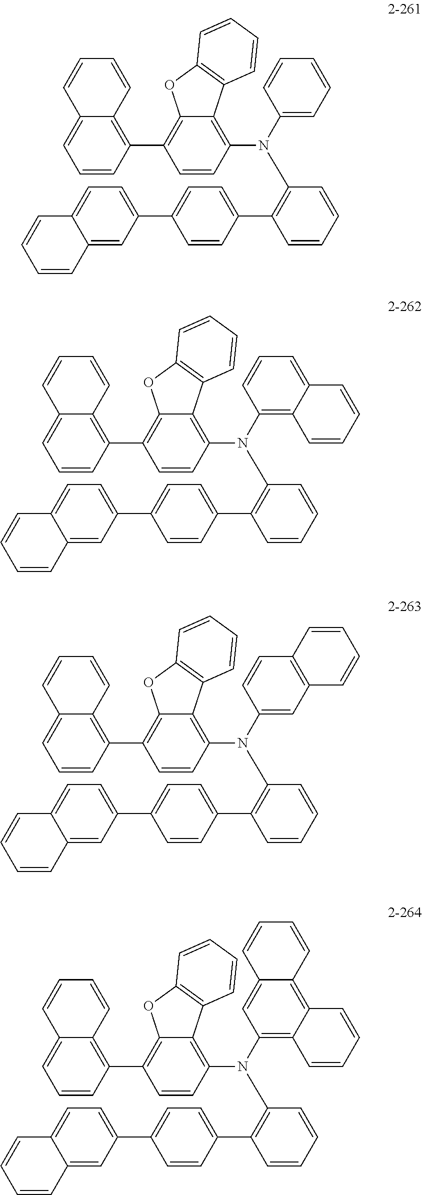 [SMILES: c1ccc(N(c2ccccc2-c2ccc(-c3ccc4ccccc4c3)cc2)c2ccc(-c3cccc4ccccc34)c3oc4ccccc4c23)cc1 ; c1ccc(N(c2cccc3ccccc23)c2ccc(-c3cccc4ccccc34)c3oc4ccccc4c23)c(-c2ccc(-c3ccc4ccccc4c3)cc2)c1 ; c1ccc(N(c2ccc3ccccc3c2)c2ccc(-c3cccc4ccccc34)c3oc4ccccc4c23)c(-c2ccc(-c3ccc4ccccc4c3)cc2)c1 ; c1ccc(N(c2cc3ccccc3c3ccccc23)c2ccc(-c3cccc4ccccc34)c3oc4ccccc4c23)c(-c2ccc(-c3ccc4ccccc4c3)cc2)c1]

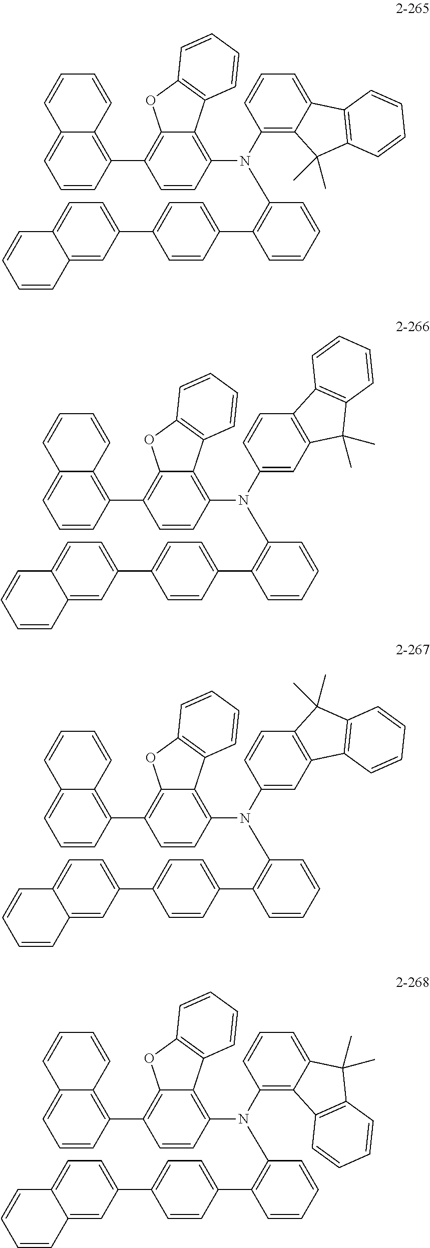 [SMILES: CC1(C)c2ccccc2-c2cccc(N(c3ccccc3-c3ccc(-c4ccc5ccccc5c4)cc3)c3ccc(-c4cccc5ccccc45)c4oc5ccccc5c34)c21 ; CC1(C)c2ccccc2-c2ccc(N(c3ccccc3-c3ccc(-c4ccc5ccccc5c4)cc3)c3ccc(-c4cccc5ccccc45)c4oc5ccccc5c34)cc21 ; CC1(C)c2ccccc2-c2cc(N(c3ccccc3-c3ccc(-c4ccc5ccccc5c4)cc3)c3ccc(-c4cccc5ccccc45)c4oc5ccccc5c34)ccc21 ; CC1(C)c2ccccc2-c2c(N(c3ccccc3-c3ccc(-c4ccc5ccccc5c4)cc3)c3ccc(-c4cccc5ccccc45)c4oc5ccccc5c34)cccc21]

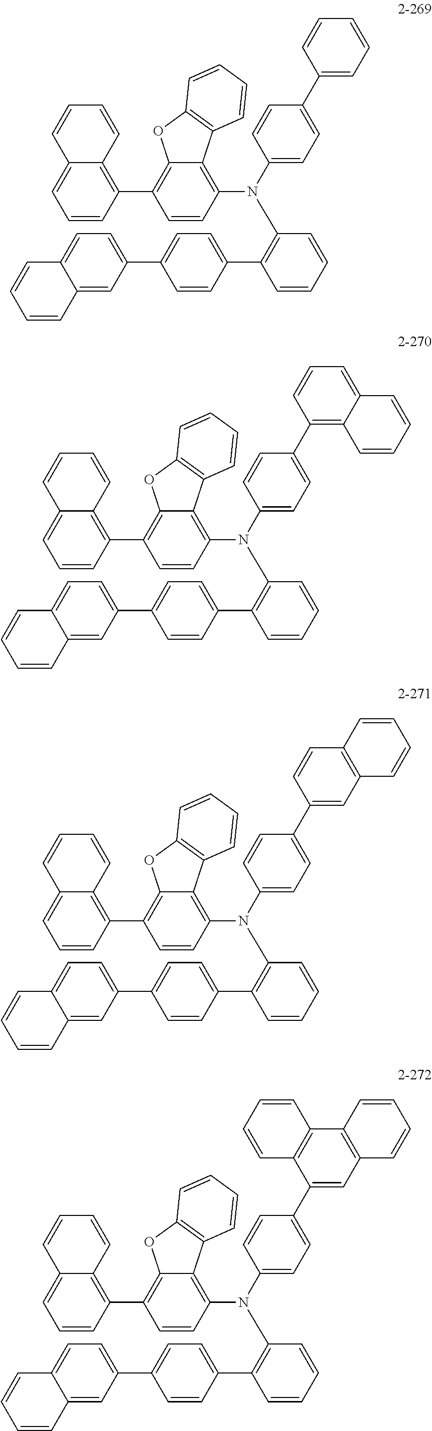 [SMILES: c1ccc(-c2ccc(N(c3ccccc3-c3ccc(-c4ccc5ccccc5c4)cc3)c3ccc(-c4cccc5ccccc45)c4oc5ccccc5c34)cc2)cc1 ; c1ccc(N(c2ccc(-c3cccc4ccccc34)cc2)c2ccc(-c3cccc4ccccc34)c3oc4ccccc4c23)c(-c2ccc(-c3ccc4ccccc4c3)cc2)c1 ; c1ccc(N(c2ccc(-c3ccc4ccccc4c3)cc2)c2ccc(-c3cccc4ccccc34)c3oc4ccccc4c23)c(-c2ccc(-c3ccc4ccccc4c3)cc2)c1 ; c1ccc(N(c2ccc(-c3cc4ccccc4c4ccccc34)cc2)c2ccc(-c3cccc4ccccc34)c3oc4ccccc4c23)c(-c2ccc(-c3ccc4ccccc4c3)cc2)c1]

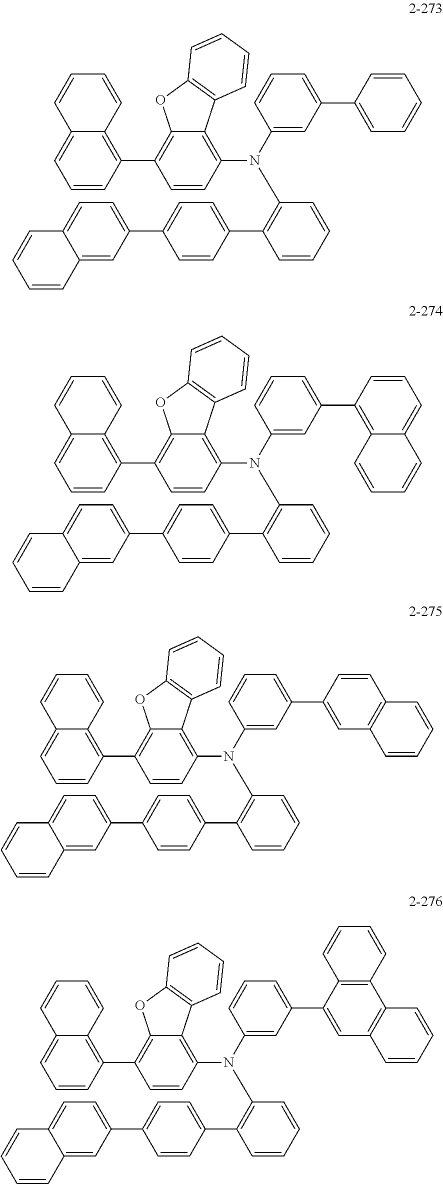 [SMILES: c1ccc(-c2cccc(N(c3ccccc3-c3ccc(-c4ccc5ccccc5c4)cc3)c3ccc(-c4cccc5ccccc45)c4oc5ccccc5c34)c2)cc1 ; c1cc(-c2cccc3ccccc23)cc(N(c2ccccc2-c2ccc(-c3ccc4ccccc4c3)cc2)c2ccc(-c3cccc4ccccc34)c3oc4ccccc4c23)c1 ; c1cc(-c2ccc3ccccc3c2)cc(N(c2ccccc2-c2ccc(-c3ccc4ccccc4c3)cc2)c2ccc(-c3cccc4ccccc34)c3oc4ccccc4c23)c1 ; c1cc(-c2cc3ccccc3c3ccccc23)cc(N(c2ccccc2-c2ccc(-c3ccc4ccccc4c3)cc2)c2ccc(-c3cccc4ccccc34)c3oc4ccccc4c23)c1]

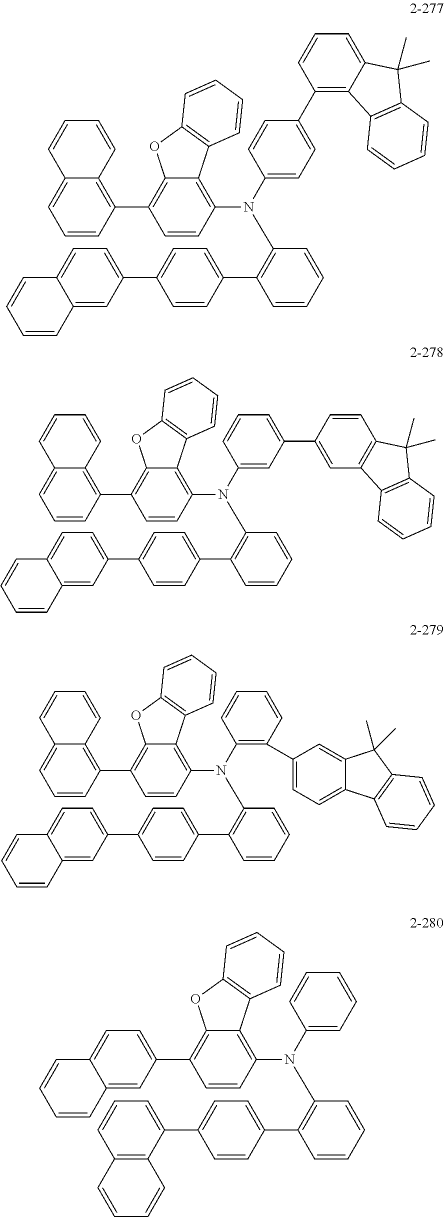 [SMILES: CC1(C)c2ccccc2-c2c(-c3ccc(N(c4ccccc4-c4ccc(-c5ccc6ccccc6c5)cc4)c4ccc(-c5cccc6ccccc56)c5oc6ccccc6c45)cc3)cccc21 ; CC1(C)c2ccccc2-c2cc(-c3cccc(N(c4ccccc4-c4ccc(-c5ccc6ccccc6c5)cc4)c4ccc(-c5cccc6ccccc56)c5oc6ccccc6c45)c3)ccc21 ; CC1(C)c2ccccc2-c2ccc(-c3ccccc3N(c3ccccc3-c3ccc(-c4ccc5ccccc5c4)cc3)c3ccc(-c4cccc5ccccc45)c4oc5ccccc5c34)cc21 ; c1ccc(N(c2ccccc2-c2ccc(-c3cccc4ccccc34)cc2)c2ccc(-c3ccc4ccccc4c3)c3oc4ccccc4c23)cc1]

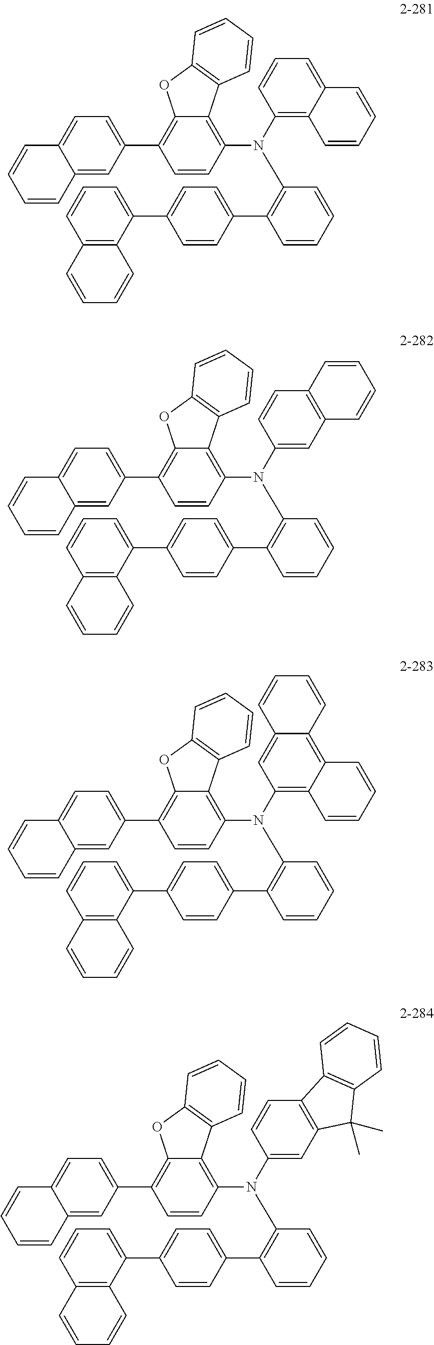 [SMILES: c1ccc(N(c2cccc3ccccc23)c2ccc(-c3ccc4ccccc4c3)c3oc4ccccc4c23)c(-c2ccc(-c3cccc4ccccc34)cc2)c1 ; c1ccc(N(c2ccc3ccccc3c2)c2ccc(-c3ccc4ccccc4c3)c3oc4ccccc4c23)c(-c2ccc(-c3cccc4ccccc34)cc2)c1 ; c1ccc(N(c2cc3ccccc3c3ccccc23)c2ccc(-c3ccc4ccccc4c3)c3oc4ccccc4c23)c(-c2ccc(-c3cccc4ccccc34)cc2)c1 ; CC1(C)c2ccccc2-c2ccc(N(c3ccccc3-c3ccc(-c4cccc5ccccc45)cc3)c3ccc(-c4ccc5ccccc5c4)c4oc5ccccc5c34)cc21]

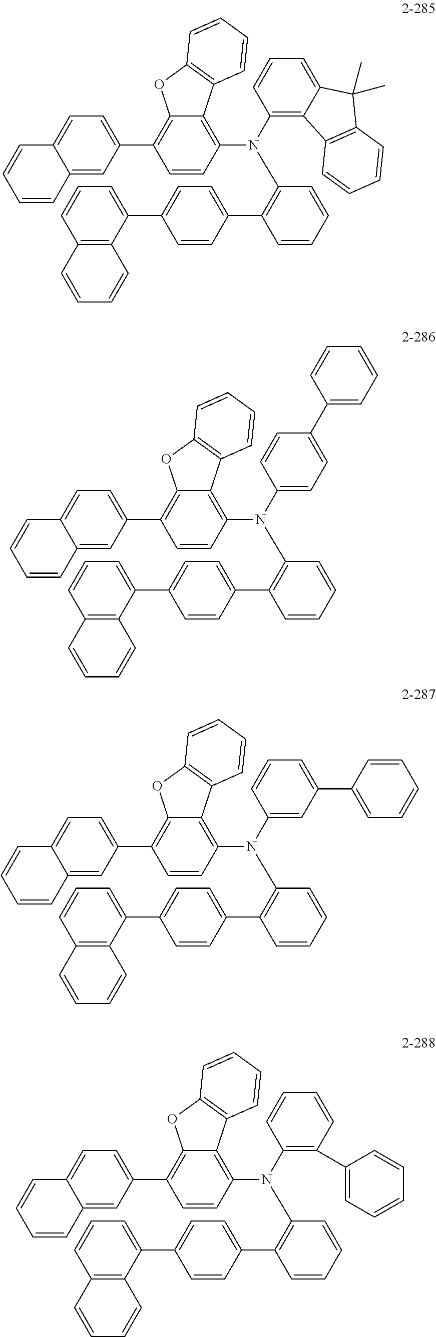 [SMILES: CC1(C)c2ccccc2-c2c(N(c3ccccc3-c3ccc(-c4cccc5ccccc45)cc3)c3ccc(-c4ccc5ccccc5c4)c4oc5ccccc5c34)cccc21 ; c1ccc(-c2ccc(N(c3ccccc3-c3ccc(-c4cccc5ccccc45)cc3)c3ccc(-c4ccc5ccccc5c4)c4oc5ccccc5c34)cc2)cc1 ; c1ccc(-c2cccc(N(c3ccccc3-c3ccc(-c4cccc5ccccc45)cc3)c3ccc(-c4ccc5ccccc5c4)c4oc5ccccc5c34)c2)cc1 ; c1ccc(-c2ccccc2N(c2ccccc2-c2ccc(-c3cccc4ccccc34)cc2)c2ccc(-c3ccc4ccccc4c3)c3oc4ccccc4c23)cc1]

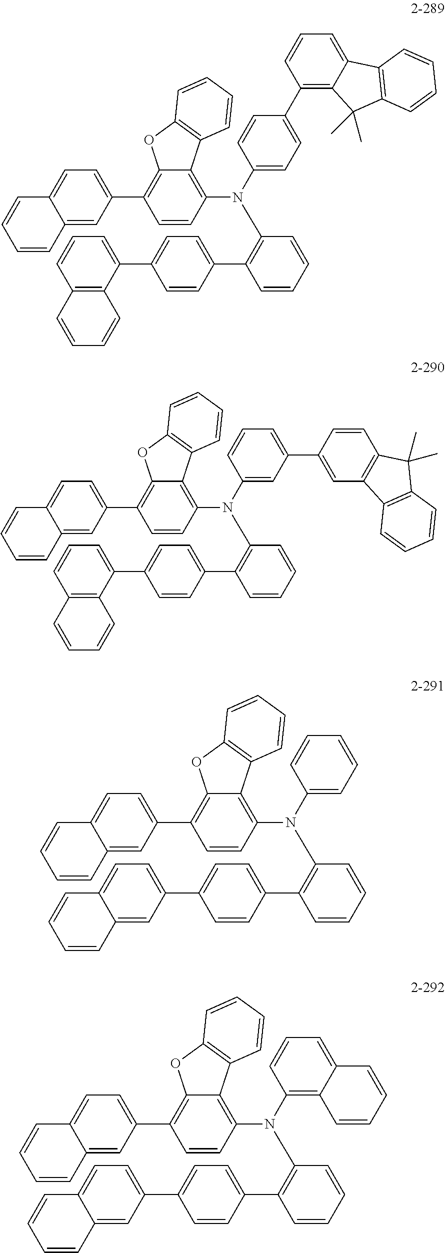 [SMILES: CC1(C)c2ccccc2-c2cccc(-c3ccc(N(c4ccccc4-c4ccc(-c5cccc6ccccc56)cc4)c4ccc(-c5ccc6ccccc6c5)c5oc6ccccc6c45)cc3)c21 ; CC1(C)c2ccccc2-c2cc(-c3cccc(N(c4ccccc4-c4ccc(-c5cccc6ccccc56)cc4)c4ccc(-c5ccc6ccccc6c5)c5oc6ccccc6c45)c3)ccc21 ; c1ccc(N(c2ccccc2-c2ccc(-c3ccc4ccccc4c3)cc2)c2ccc(-c3ccc4ccccc4c3)c3oc4ccccc4c23)cc1 ; c1ccc(N(c2cccc3ccccc23)c2ccc(-c3ccc4ccccc4c3)c3oc4ccccc4c23)c(-c2ccc(-c3ccc4ccccc4c3)cc2)c1]

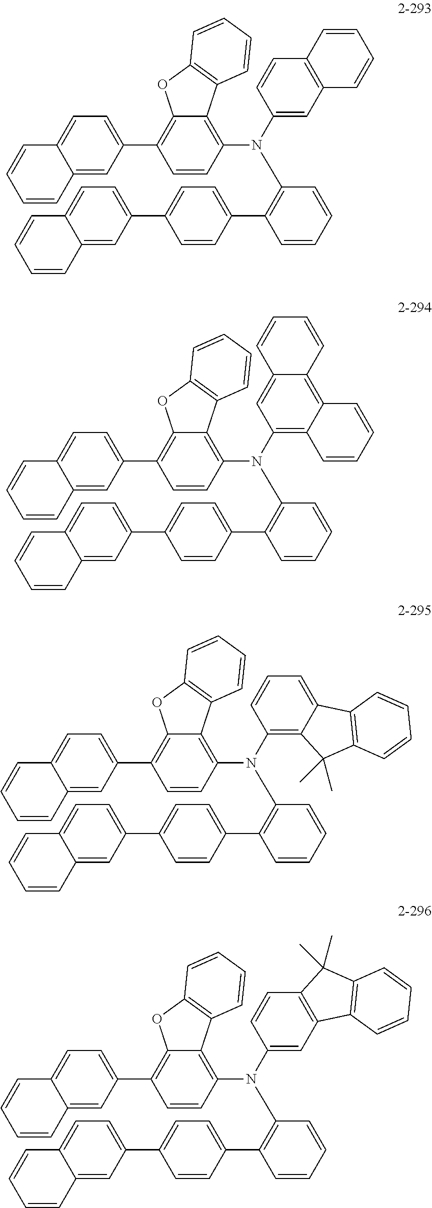 [SMILES: c1ccc(N(c2ccc3ccccc3c2)c2ccc(-c3ccc4ccccc4c3)c3oc4ccccc4c23)c(-c2ccc(-c3ccc4ccccc4c3)cc2)c1 ; c1ccc(N(c2cc3ccccc3c3ccccc23)c2ccc(-c3ccc4ccccc4c3)c3oc4ccccc4c23)c(-c2ccc(-c3ccc4ccccc4c3)cc2)c1 ; CC1(C)c2ccccc2-c2cccc(N(c3ccccc3-c3ccc(-c4ccc5ccccc5c4)cc3)c3ccc(-c4ccc5ccccc5c4)c4oc5ccccc5c34)c21 ; CC1(C)c2ccccc2-c2cc(N(c3ccccc3-c3ccc(-c4ccc5ccccc5c4)cc3)c3ccc(-c4ccc5ccccc5c4)c4oc5ccccc5c34)ccc21]

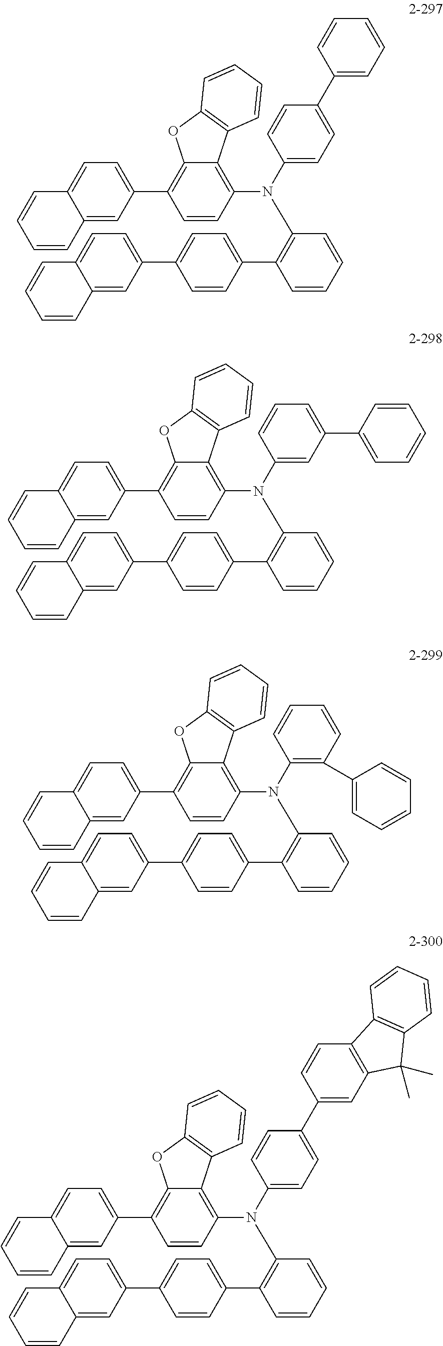 [SMILES: c1ccc(-c2ccc(N(c3ccccc3-c3ccc(-c4ccc5ccccc5c4)cc3)c3ccc(-c4ccc5ccccc5c4)c4oc5ccccc5c34)cc2)cc1 ; c1ccc(-c2cccc(N(c3ccccc3-c3ccc(-c4ccc5ccccc5c4)cc3)c3ccc(-c4ccc5ccccc5c4)c4oc5ccccc5c34)c2)cc1 ; c1ccc(-c2ccccc2N(c2ccccc2-c2ccc(-c3ccc4ccccc4c3)cc2)c2ccc(-c3ccc4ccccc4c3)c3oc4ccccc4c23)cc1 ; CC1(C)c2ccccc2-c2ccc(-c3ccc(N(c4ccccc4-c4ccc(-c5ccc6ccccc6c5)cc4)c4ccc(-c5ccc6ccccc6c5)c5oc6ccccc6c45)cc3)cc21]

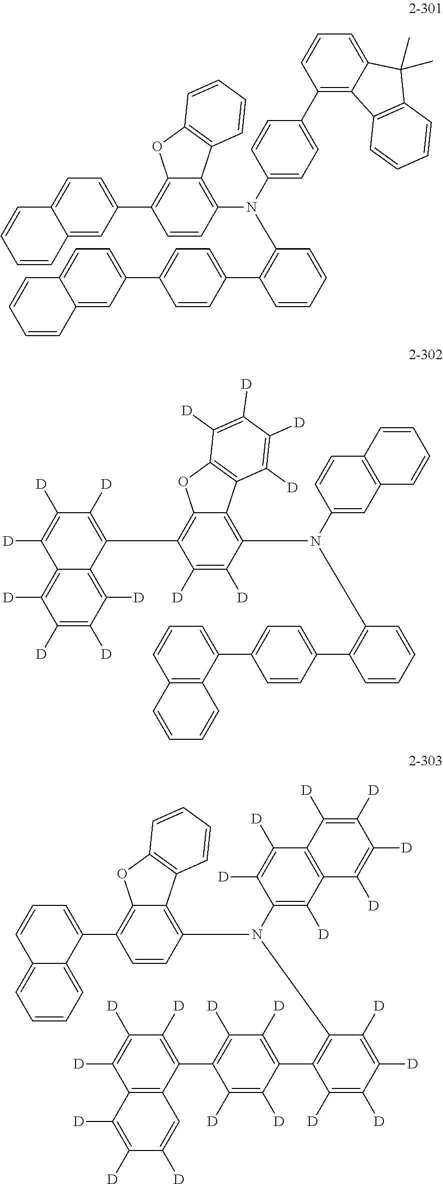 [SMILES: CC1(C)c2ccccc2-c2c(-c3ccc(N(c4ccccc4-c4ccc(-c5ccc6ccccc6c5)cc4)c4ccc(-c5ccc6ccccc6c5)c5oc6ccccc6c45)cc3)cccc21 ; [2H]c1c([2H])c([2H])c2c(oc3c(-c4c([2H])c([2H])c([2H])c5c([2H])c([2H])c([2H])c([2H])c45)c([2H])c([2H])c(N(c4ccc5ccccc5c4)c4ccccc4-c4ccc(-c5cccc6ccccc56)cc4)c32)c1[2H] ; [2H]c1cc2c(-c3c([2H])c([2H])c(-c4c([2H])c([2H])c([2H])c([2H])c4N(c4c([2H])c([2H])c5c([2H])c([2H])c([2H])c([2H])c5c4[2H])c4ccc(-c5cccc6ccccc56)c5oc6ccccc6c45)c([2H])c3[2H])c([2H])c([2H])c([2H])c2c([2H])c1[2H]]

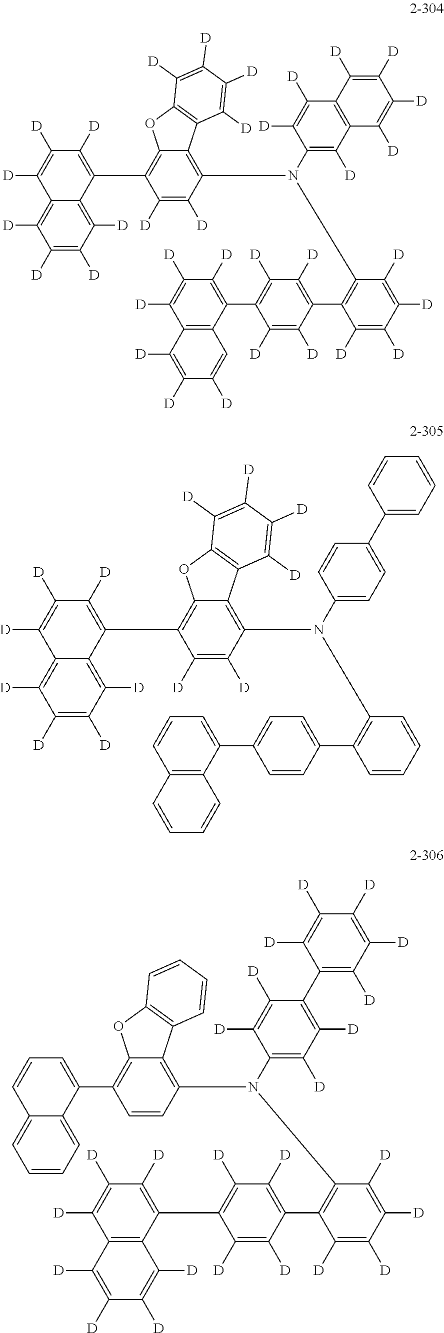

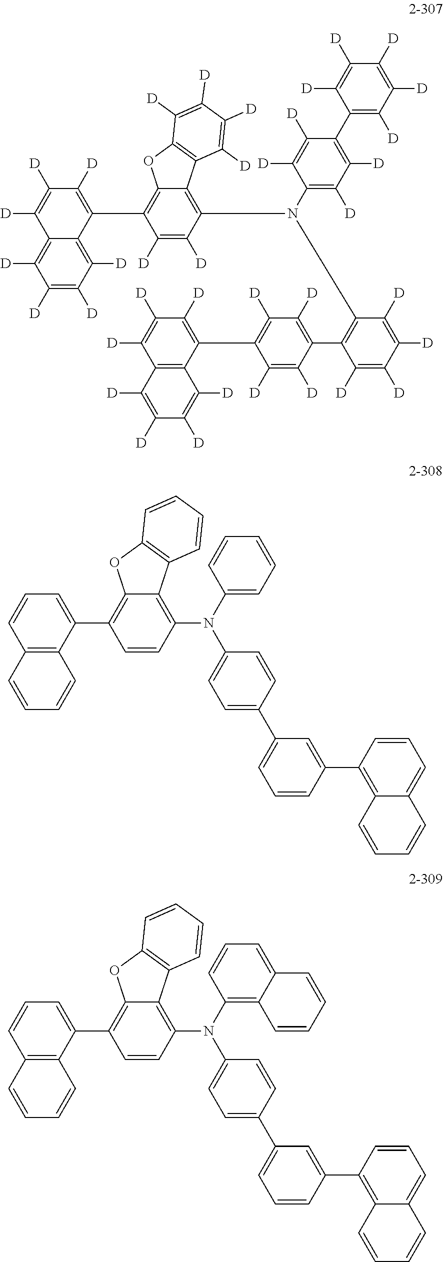 [SMILES: [2H]c1c([2H])c([2H])c(-c2c([2H])c([2H])c(N(c3c([2H])c([2H])c([2H])c([2H])c3-c3c([2H])c([2H])c(-c4c([2H])c([2H])c([2H])c5c([2H])c([2H])c([2H])c([2H])c45)c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H] ; c1ccc(N(c2ccc(-c3cccc(-c4cccc5ccccc45)c3)cc2)c2ccc(-c3cccc4ccccc34)c3oc4ccccc4c23)cc1 ; c1cc(-c2ccc(N(c3cccc4ccccc34)c3ccc(-c4cccc5ccccc45)c4oc5ccccc5c34)cc2)cc(-c2cccc3ccccc23)c1]

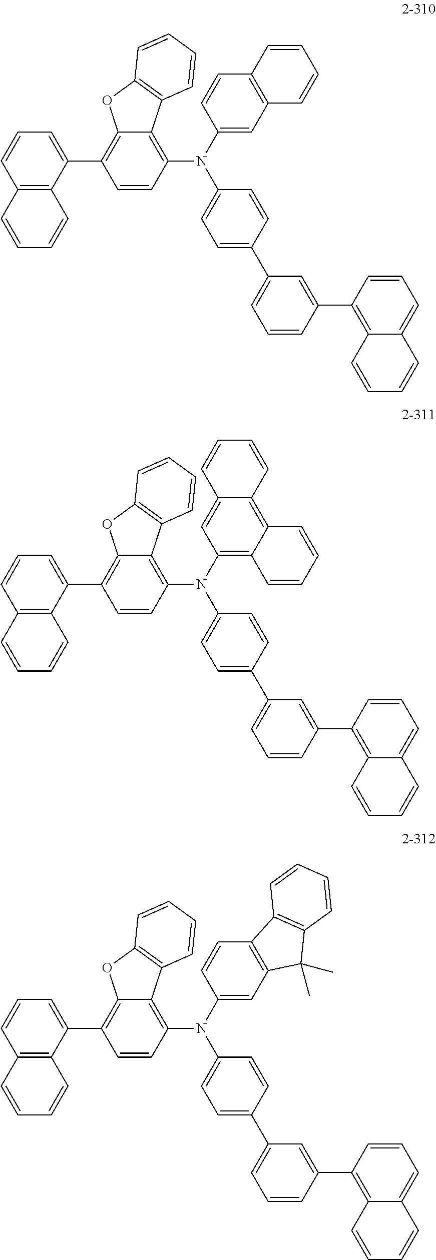 [SMILES: c1cc(-c2ccc(N(c3ccc4ccccc4c3)c3ccc(-c4cccc5ccccc45)c4oc5ccccc5c34)cc2)cc(-c2cccc3ccccc23)c1 ; c1cc(-c2ccc(N(c3cc4ccccc4c4ccccc34)c3ccc(-c4cccc5ccccc45)c4oc5ccccc5c34)cc2)cc(-c2cccc3ccccc23)c1 ; CC1(C)c2ccccc2-c2ccc(N(c3ccc(-c4cccc(-c5cccc6ccccc56)c4)cc3)c3ccc(-c4cccc5ccccc45)c4oc5ccccc5c34)cc21]

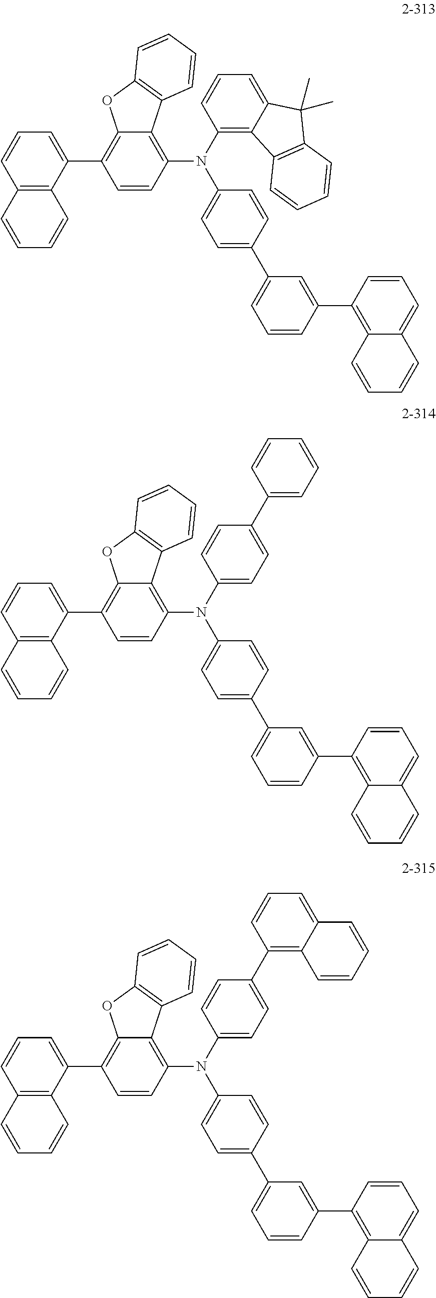 [SMILES: CC1(C)c2ccccc2-c2c(N(c3ccc(-c4cccc(-c5cccc6ccccc56)c4)cc3)c3ccc(-c4cccc5ccccc45)c4oc5ccccc5c34)cccc21 ; c1ccc(-c2ccc(N(c3ccc(-c4cccc(-c5cccc6ccccc56)c4)cc3)c3ccc(-c4cccc5ccccc45)c4oc5ccccc5c34)cc2)cc1 ; c1cc(-c2ccc(N(c3ccc(-c4cccc5ccccc45)cc3)c3ccc(-c4cccc5ccccc45)c4oc5ccccc5c34)cc2)cc(-c2cccc3ccccc23)c1]

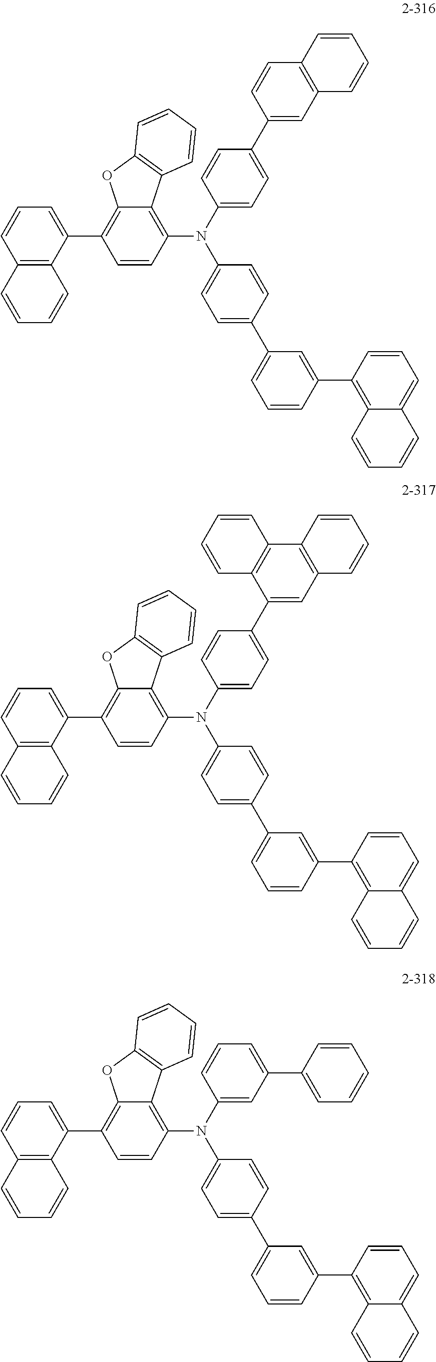 [SMILES: c1cc(-c2ccc(N(c3ccc(-c4ccc5ccccc5c4)cc3)c3ccc(-c4cccc5ccccc45)c4oc5ccccc5c34)cc2)cc(-c2cccc3ccccc23)c1 ; c1cc(-c2ccc(N(c3ccc(-c4cc5ccccc5c5ccccc45)cc3)c3ccc(-c4cccc5ccccc45)c4oc5ccccc5c34)cc2)cc(-c2cccc3ccccc23)c1 ; c1ccc(-c2cccc(N(c3ccc(-c4cccc(-c5cccc6ccccc56)c4)cc3)c3ccc(-c4cccc5ccccc45)c4oc5ccccc5c34)c2)cc1]

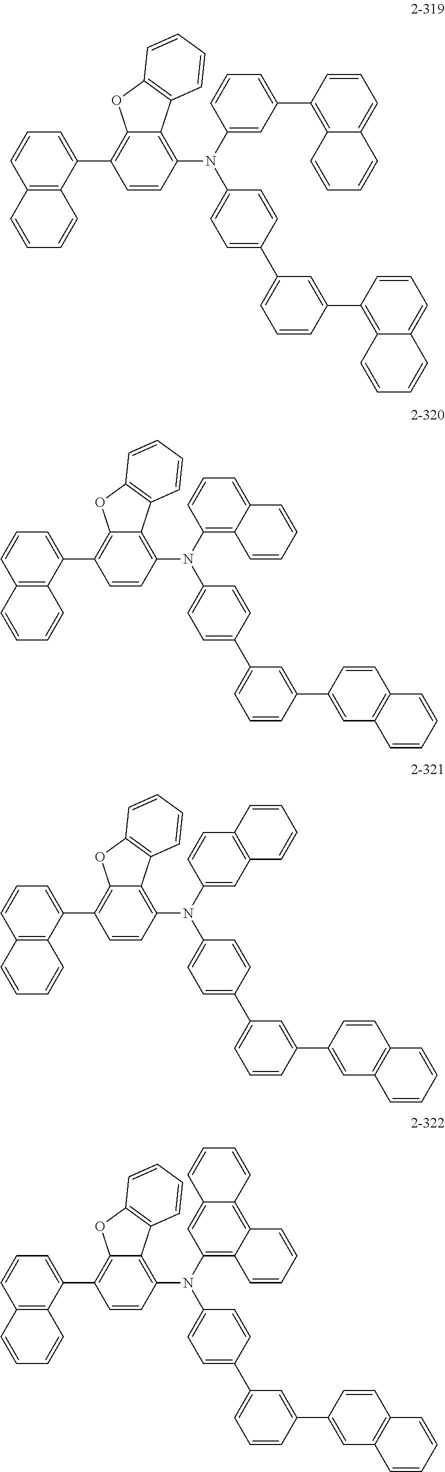 [SMILES: c1cc(-c2ccc(N(c3cccc(-c4cccc5ccccc45)c3)c3ccc(-c4cccc5ccccc45)c4oc5ccccc5c34)cc2)cc(-c2cccc3ccccc23)c1 ; c1cc(-c2ccc(N(c3cccc4ccccc34)c3ccc(-c4cccc5ccccc45)c4oc5ccccc5c34)cc2)cc(-c2ccc3ccccc3c2)c1 ; c1cc(-c2ccc(N(c3ccc4ccccc4c3)c3ccc(-c4cccc5ccccc45)c4oc5ccccc5c34)cc2)cc(-c2ccc3ccccc3c2)c1 ; c1cc(-c2ccc(N(c3cc4ccccc4c4ccccc34)c3ccc(-c4cccc5ccccc45)c4oc5ccccc5c34)cc2)cc(-c2ccc3ccccc3c2)c1]

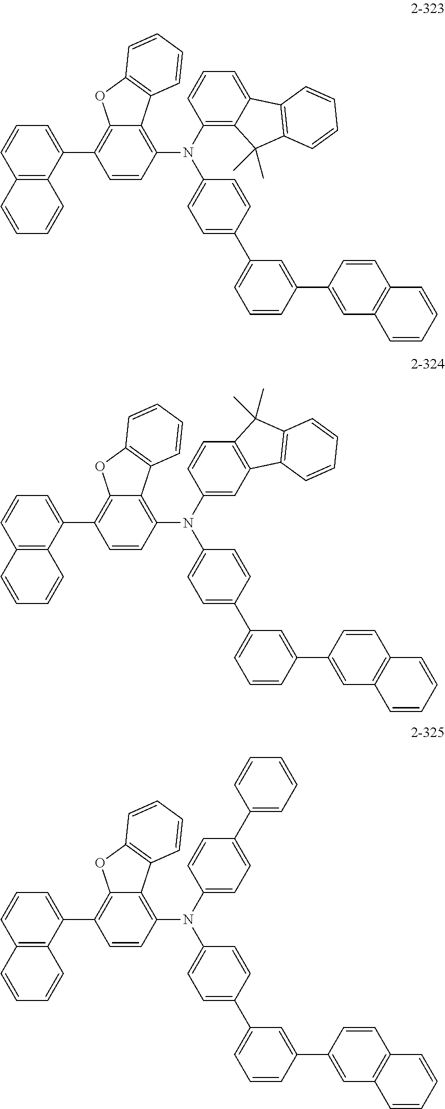 [SMILES: CC1(C)c2ccccc2-c2cccc(N(c3ccc(-c4cccc(-c5ccc6ccccc6c5)c4)cc3)c3ccc(-c4cccc5ccccc45)c4oc5ccccc5c34)c21 ; CC1(C)c2ccccc2-c2cc(N(c3ccc(-c4cccc(-c5ccc6ccccc6c5)c4)cc3)c3ccc(-c4cccc5ccccc45)c4oc5ccccc5c34)ccc21 ; c1ccc(-c2ccc(N(c3ccc(-c4cccc(-c5ccc6ccccc6c5)c4)cc3)c3ccc(-c4cccc5ccccc45)c4oc5ccccc5c34)cc2)cc1]

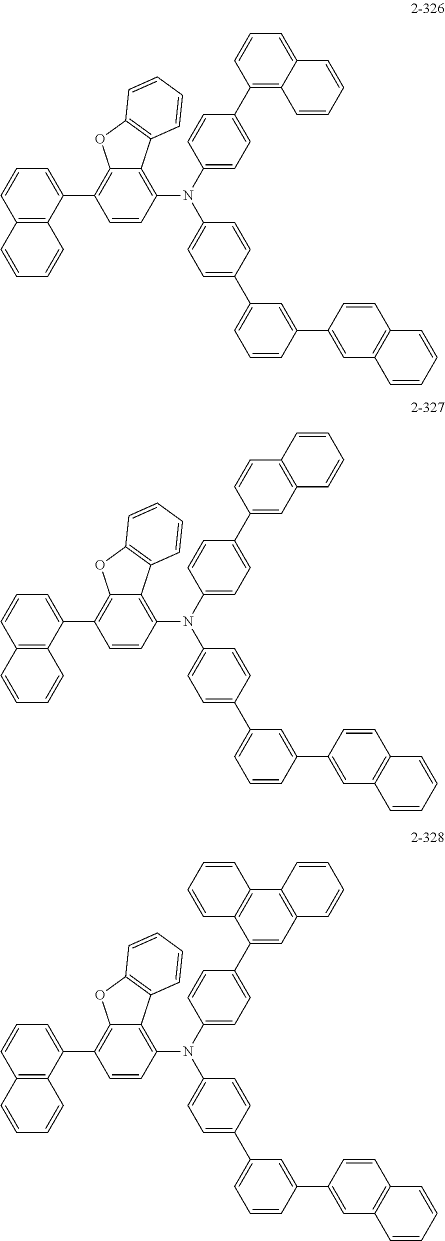 [SMILES: c1cc(-c2ccc(N(c3ccc(-c4cccc5ccccc45)cc3)c3ccc(-c4cccc5ccccc45)c4oc5ccccc5c34)cc2)cc(-c2ccc3ccccc3c2)c1 ; c1cc(-c2ccc(N(c3ccc(-c4ccc5ccccc5c4)cc3)c3ccc(-c4cccc5ccccc45)c4oc5ccccc5c34)cc2)cc(-c2ccc3ccccc3c2)c1 ; c1cc(-c2ccc(N(c3ccc(-c4cc5ccccc5c5ccccc45)cc3)c3ccc(-c4cccc5ccccc45)c4oc5ccccc5c34)cc2)cc(-c2ccc3ccccc3c2)c1]

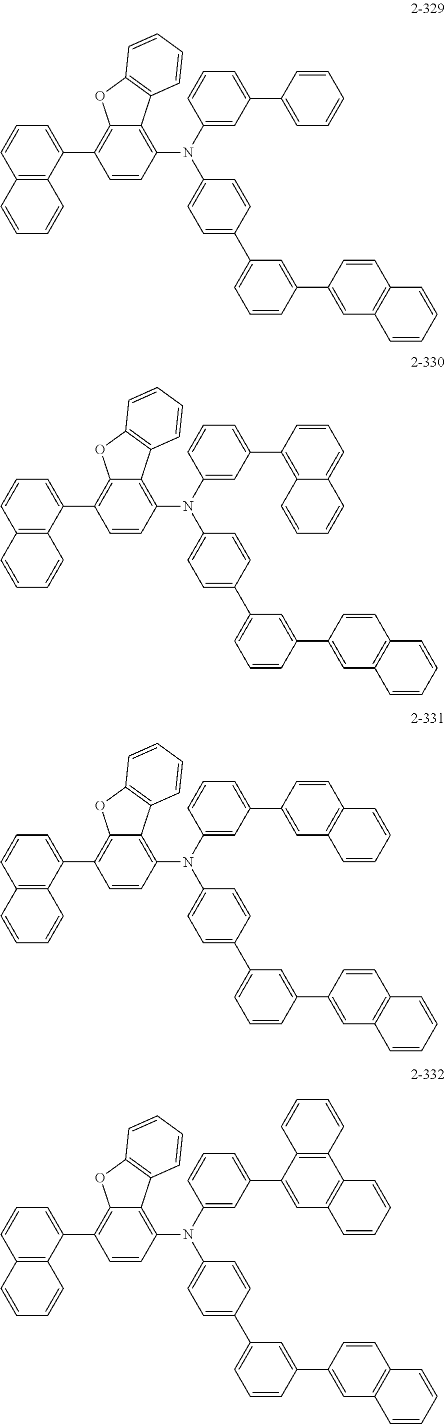 [SMILES: c1ccc(-c2cccc(N(c3ccc(-c4cccc(-c5ccc6ccccc6c5)c4)cc3)c3ccc(-c4cccc5ccccc45)c4oc5ccccc5c34)c2)cc1 ; c1cc(-c2ccc(N(c3cccc(-c4cccc5ccccc45)c3)c3ccc(-c4cccc5ccccc45)c4oc5ccccc5c34)cc2)cc(-c2ccc3ccccc3c2)c1 ; c1cc(-c2ccc(N(c3cccc(-c4ccc5ccccc5c4)c3)c3ccc(-c4cccc5ccccc45)c4oc5ccccc5c34)cc2)cc(-c2ccc3ccccc3c2)c1 ; c1cc(-c2ccc(N(c3cccc(-c4cc5ccccc5c5ccccc45)c3)c3ccc(-c4cccc5ccccc45)c4oc5ccccc5c34)cc2)cc(-c2ccc3ccccc3c2)c1]

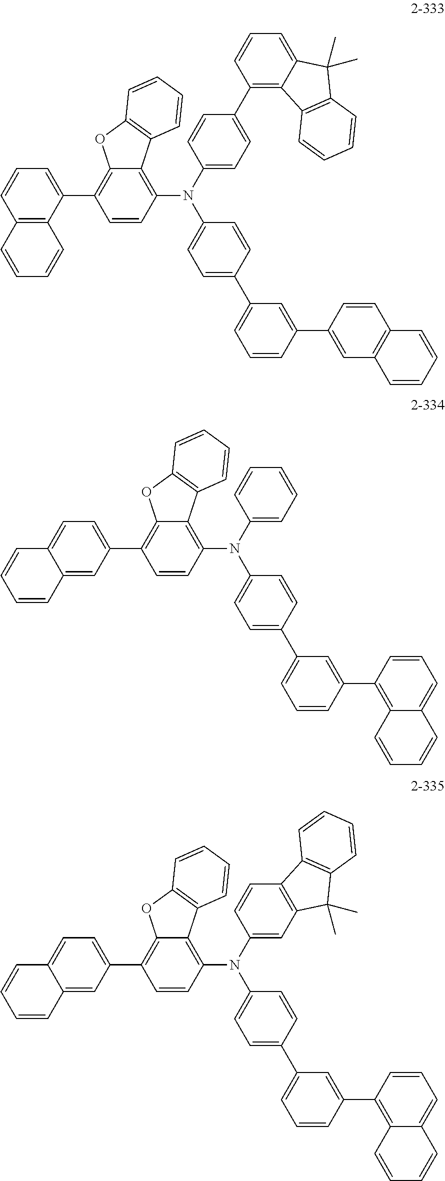 [SMILES: CC1(C)c2ccccc2-c2c(-c3ccc(N(c4ccc(-c5cccc(-c6ccc7ccccc7c6)c5)cc4)c4ccc(-c5cccc6ccccc56)c5oc6ccccc6c45)cc3)cccc21 ; c1ccc(N(c2ccc(-c3cccc(-c4cccc5ccccc45)c3)cc2)c2ccc(-c3ccc4ccccc4c3)c3oc4ccccc4c23)cc1 ; CC1(C)c2ccccc2-c2ccc(N(c3ccc(-c4cccc(-c5cccc6ccccc56)c4)cc3)c3ccc(-c4ccc5ccccc5c4)c4oc5ccccc5c34)cc21]

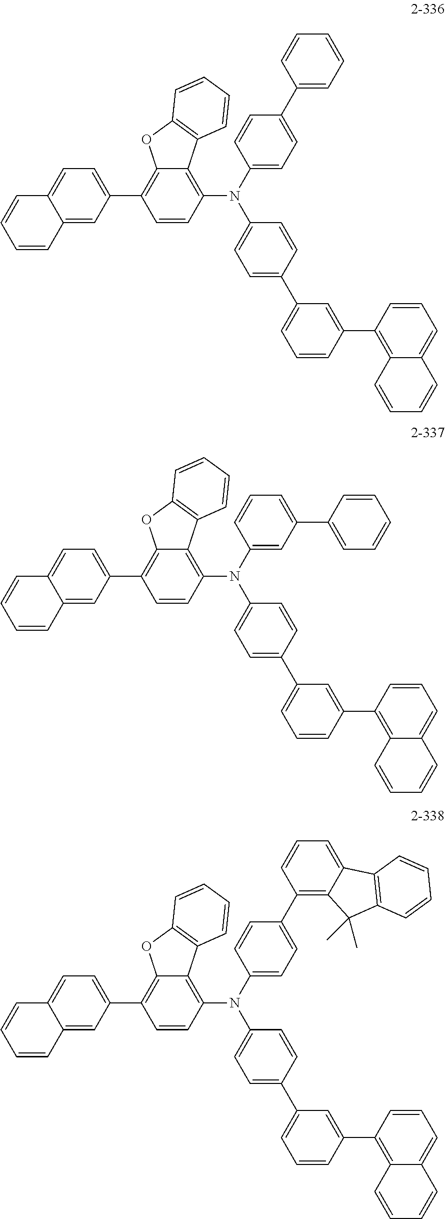 [SMILES: c1ccc(-c2ccc(N(c3ccc(-c4cccc(-c5cccc6ccccc56)c4)cc3)c3ccc(-c4ccc5ccccc5c4)c4oc5ccccc5c34)cc2)cc1 ; c1ccc(-c2cccc(N(c3ccc(-c4cccc(-c5cccc6ccccc56)c4)cc3)c3ccc(-c4ccc5ccccc5c4)c4oc5ccccc5c34)c2)cc1 ; CC1(C)c2ccccc2-c2cccc(-c3ccc(N(c4ccc(-c5cccc(-c6cccc7ccccc67)c5)cc4)c4ccc(-c5ccc6ccccc6c5)c5oc6ccccc6c45)cc3)c21]

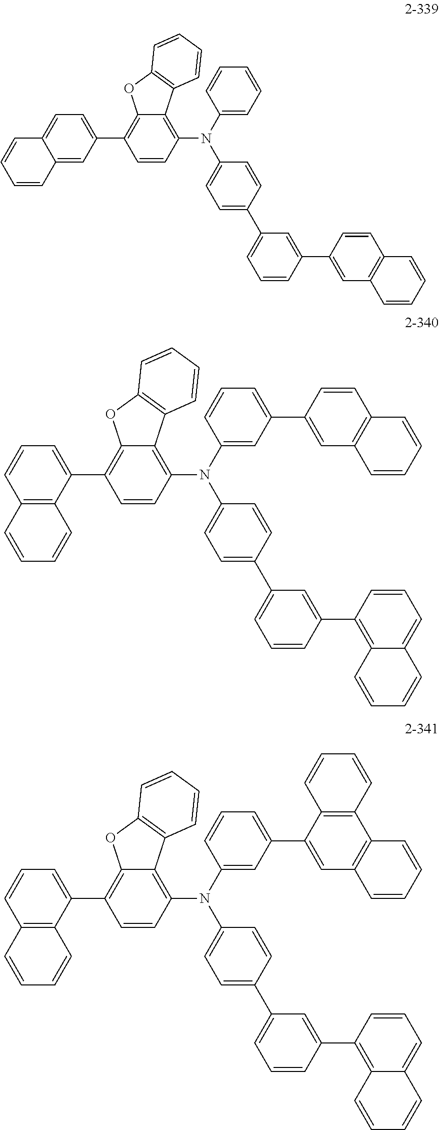 [SMILES: c1ccc(N(c2ccc(-c3cccc(-c4ccc5ccccc5c4)c3)cc2)c2ccc(-c3ccc4ccccc4c3)c3oc4ccccc4c23)cc1 ; c1cc(-c2ccc(N(c3cccc(-c4ccc5ccccc5c4)c3)c3ccc(-c4cccc5ccccc45)c4oc5ccccc5c34)cc2)cc(-c2cccc3ccccc23)c1 ; c1cc(-c2ccc(N(c3cccc(-c4cc5ccccc5c5ccccc45)c3)c3ccc(-c4cccc5ccccc45)c4oc5ccccc5c34)cc2)cc(-c2cccc3ccccc23)c1]

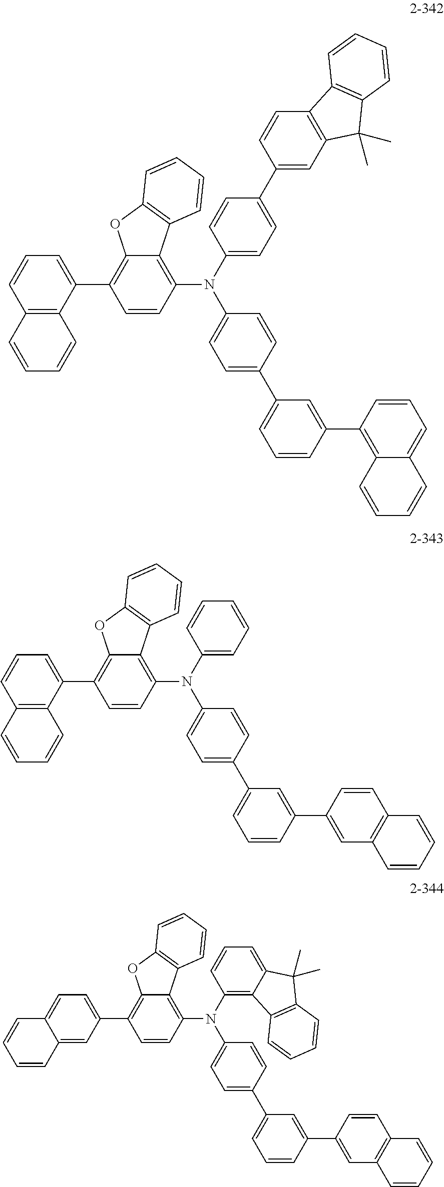 [SMILES: CC1(C)c2ccccc2-c2ccc(-c3ccc(N(c4ccc(-c5cccc(-c6cccc7ccccc67)c5)cc4)c4ccc(-c5cccc6ccccc56)c5oc6ccccc6c45)cc3)cc21 ; c1ccc(N(c2ccc(-c3cccc(-c4ccc5ccccc5c4)c3)cc2)c2ccc(-c3cccc4ccccc34)c3oc4ccccc4c23)cc1 ; CC1(C)c2ccccc2-c2c(N(c3ccc(-c4cccc(-c5ccc6ccccc6c5)c4)cc3)c3ccc(-c4ccc5ccccc5c4)c4oc5ccccc5c34)cccc21]

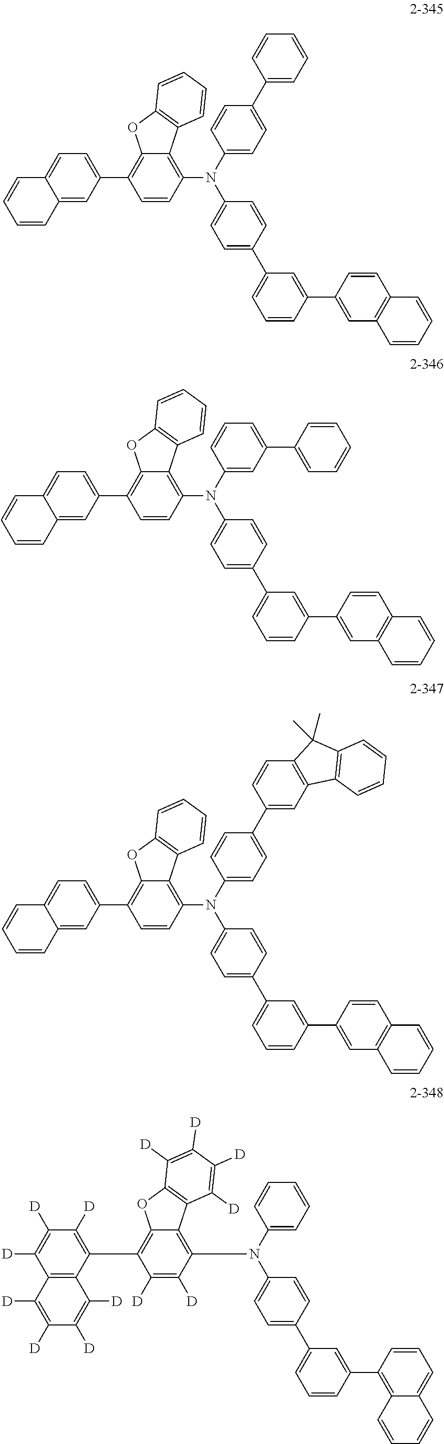 [SMILES: c1ccc(-c2ccc(N(c3ccc(-c4cccc(-c5ccc6ccccc6c5)c4)cc3)c3ccc(-c4ccc5ccccc5c4)c4oc5ccccc5c34)cc2)cc1 ; c1ccc(-c2cccc(N(c3ccc(-c4cccc(-c5ccc6ccccc6c5)c4)cc3)c3ccc(-c4ccc5ccccc5c4)c4oc5ccccc5c34)c2)cc1 ; CC1(C)c2ccccc2-c2cc(-c3ccc(N(c4ccc(-c5cccc(-c6ccc7ccccc7c6)c5)cc4)c4ccc(-c5ccc6ccccc6c5)c5oc6ccccc6c45)cc3)ccc21 ; [2H]c1c([2H])c([2H])c2c(oc3c(-c4c([2H])c([2H])c([2H])c5c([2H])c([2H])c([2H])c([2H])c45)c([2H])c([2H])c(N(c4ccccc4)c4ccc(-c5cccc(-c6cccc7ccccc67)c5)cc4)c32)c1[2H]]

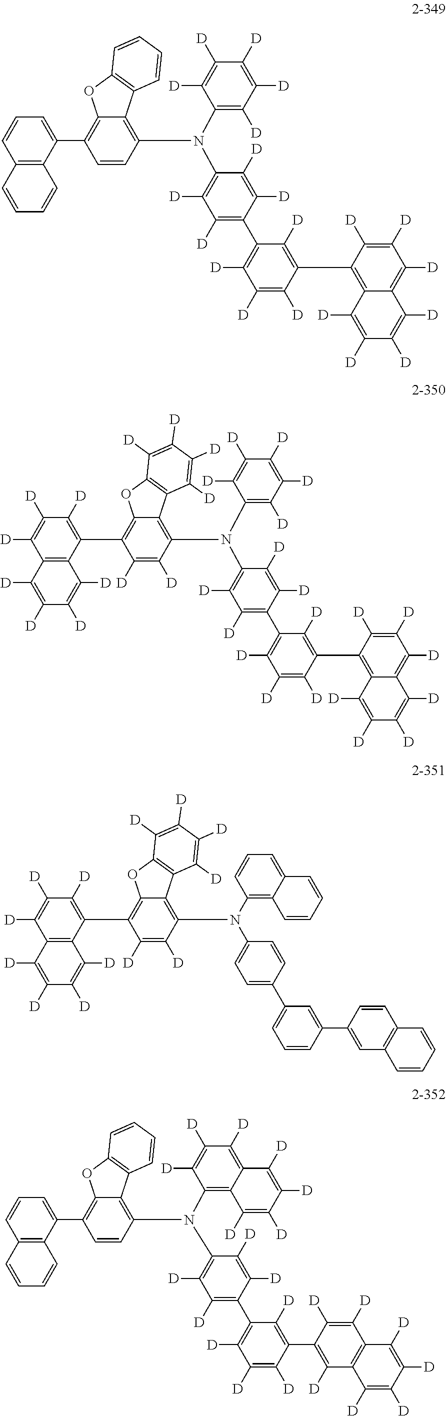

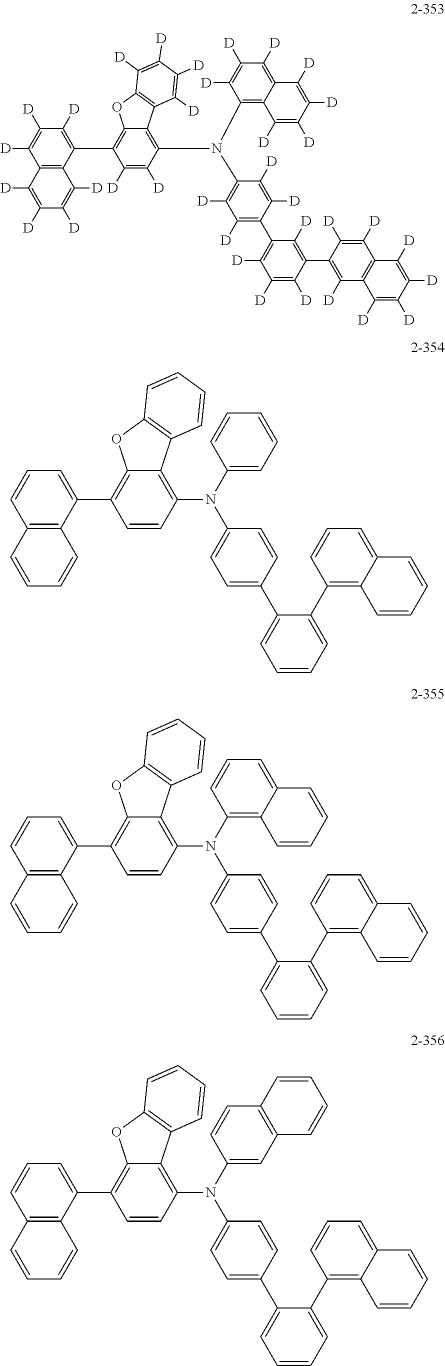

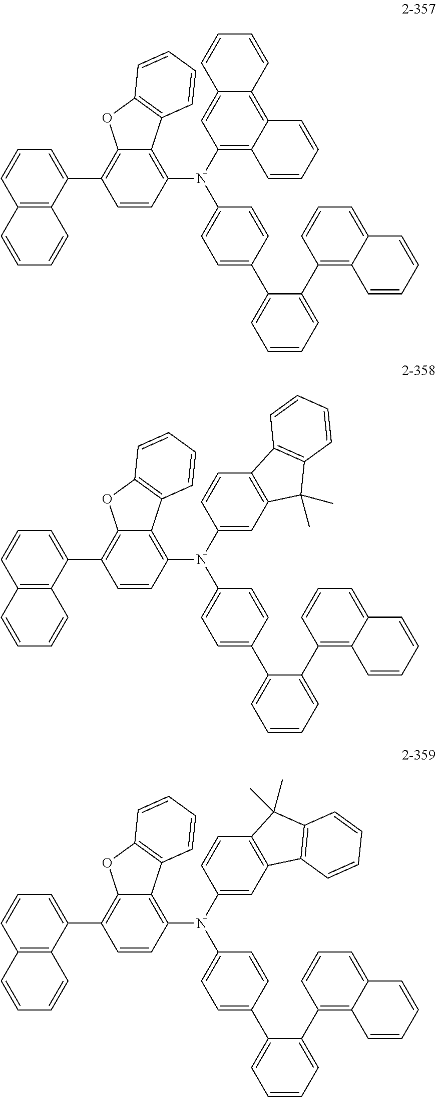

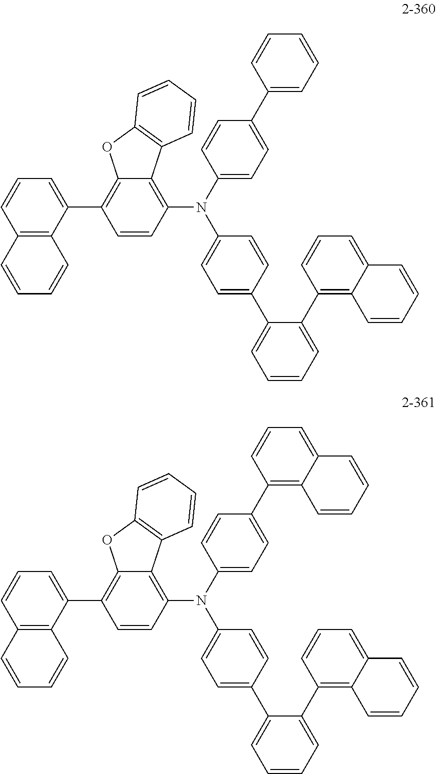 [SMILES: c1ccc(-c2ccc(N(c3ccc(-c4ccccc4-c4cccc5ccccc45)cc3)c3ccc(-c4cccc5ccccc45)c4oc5ccccc5c34)cc2)cc1 ; c1ccc(-c2cccc3ccccc23)c(-c2ccc(N(c3ccc(-c4cccc5ccccc45)cc3)c3ccc(-c4cccc5ccccc45)c4oc5ccccc5c34)cc2)c1]

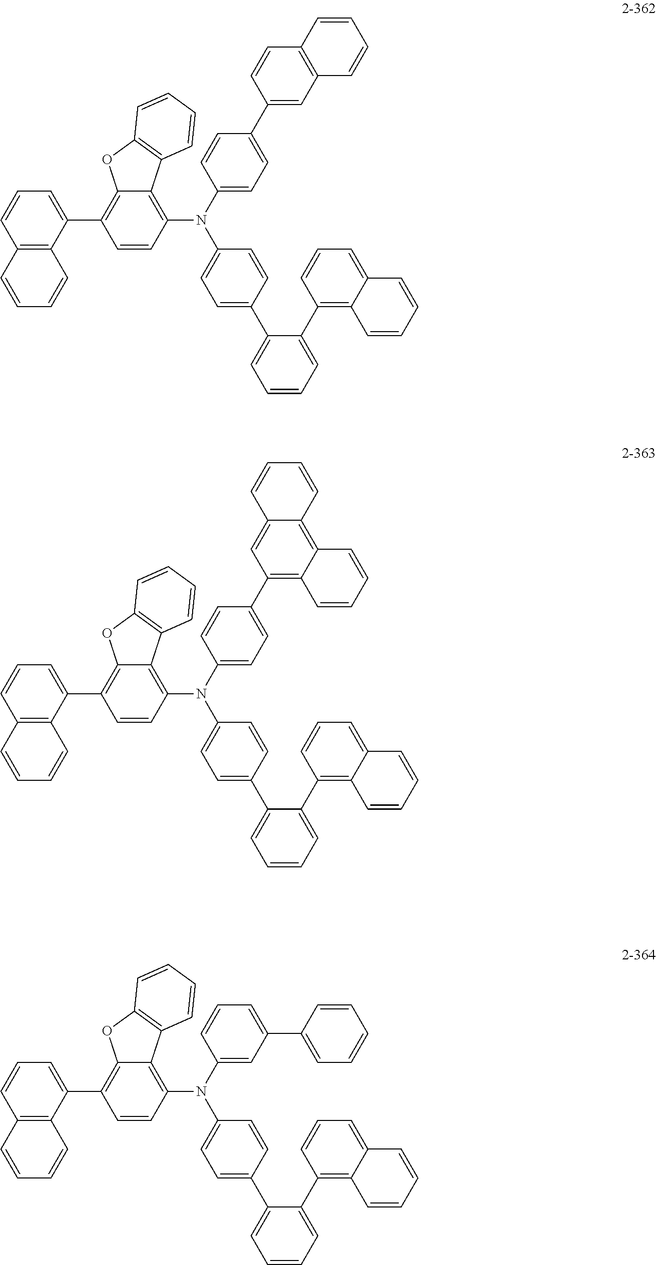 [SMILES: c1ccc(-c2cccc3ccccc23)c(-c2ccc(N(c3ccc(-c4ccc5ccccc5c4)cc3)c3ccc(-c4cccc5ccccc45)c4oc5ccccc5c34)cc2)c1 ; c1ccc(-c2cccc3ccccc23)c(-c2ccc(N(c3ccc(-c4cc5ccccc5c5ccccc45)cc3)c3ccc(-c4cccc5ccccc45)c4oc5ccccc5c34)cc2)c1 ; c1ccc(-c2cccc(N(c3ccc(-c4ccccc4-c4cccc5ccccc45)cc3)c3ccc(-c4cccc5ccccc45)c4oc5ccccc5c34)c2)cc1]

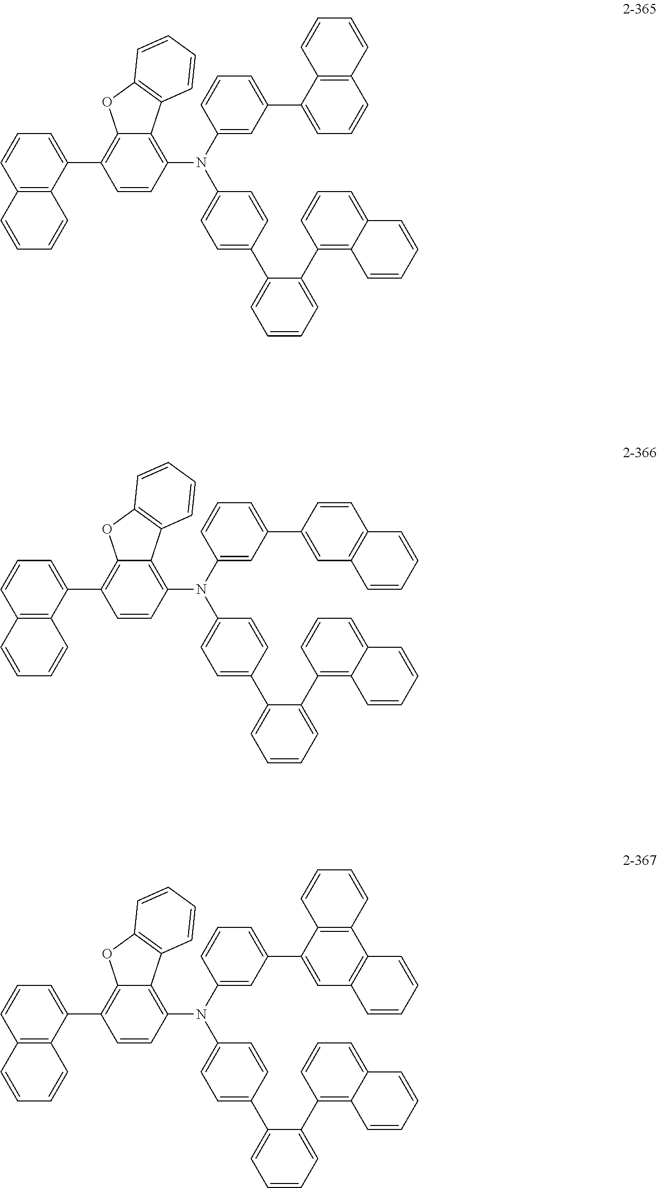 [SMILES: c1cc(-c2cccc3ccccc23)cc(N(c2ccc(-c3ccccc3-c3cccc4ccccc34)cc2)c2ccc(-c3cccc4ccccc34)c3oc4ccccc4c23)c1 ; c1cc(-c2ccc3ccccc3c2)cc(N(c2ccc(-c3ccccc3-c3cccc4ccccc34)cc2)c2ccc(-c3cccc4ccccc34)c3oc4ccccc4c23)c1 ; c1cc(-c2cc3ccccc3c3ccccc23)cc(N(c2ccc(-c3ccccc3-c3cccc4ccccc34)cc2)c2ccc(-c3cccc4ccccc34)c3oc4ccccc4c23)c1]

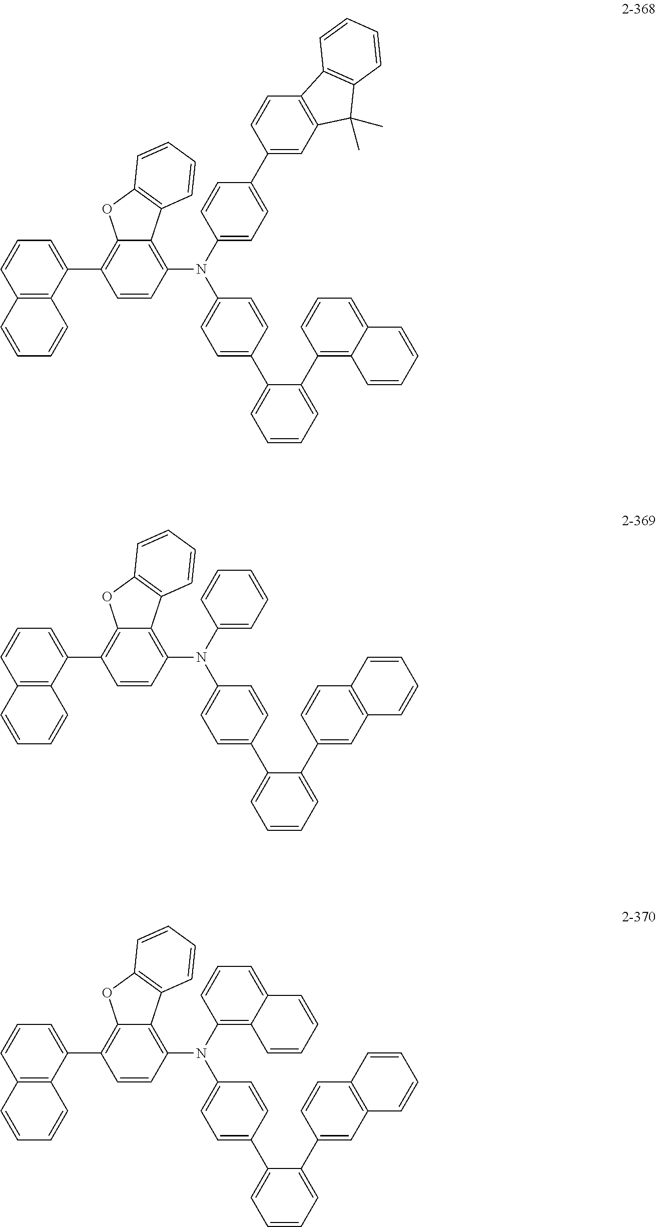 [SMILES: CC1(C)c2ccccc2-c2ccc(-c3ccc(N(c4ccc(-c5ccccc5-c5cccc6ccccc56)cc4)c4ccc(-c5cccc6ccccc56)c5oc6ccccc6c45)cc3)cc21 ; c1ccc(N(c2ccc(-c3ccccc3-c3ccc4ccccc4c3)cc2)c2ccc(-c3cccc4ccccc34)c3oc4ccccc4c23)cc1 ; c1ccc(-c2ccc3ccccc3c2)c(-c2ccc(N(c3cccc4ccccc34)c3ccc(-c4cccc5ccccc45)c4oc5ccccc5c34)cc2)c1]

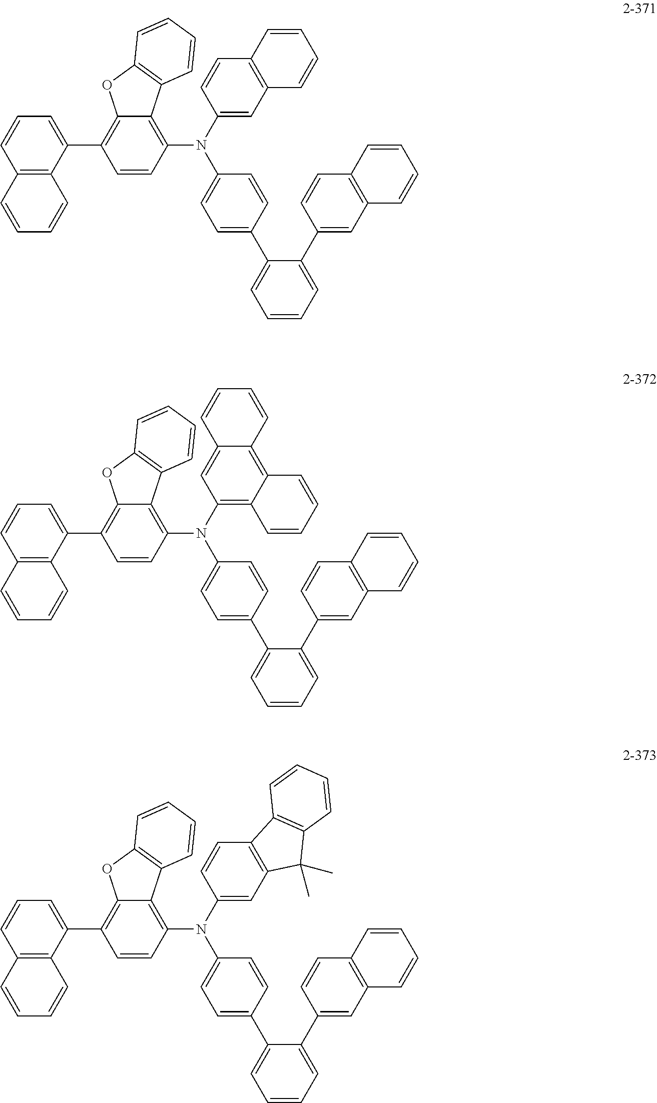 [SMILES: c1ccc(-c2ccc3ccccc3c2)c(-c2ccc(N(c3ccc4ccccc4c3)c3ccc(-c4cccc5ccccc45)c4oc5ccccc5c34)cc2)c1 ; c1ccc(-c2ccc3ccccc3c2)c(-c2ccc(N(c3cc4ccccc4c4ccccc34)c3ccc(-c4cccc5ccccc45)c4oc5ccccc5c34)cc2)c1 ; CC1(C)c2ccccc2-c2ccc(N(c3ccc(-c4ccccc4-c4ccc5ccccc5c4)cc3)c3ccc(-c4cccc5ccccc45)c4oc5ccccc5c34)cc21]

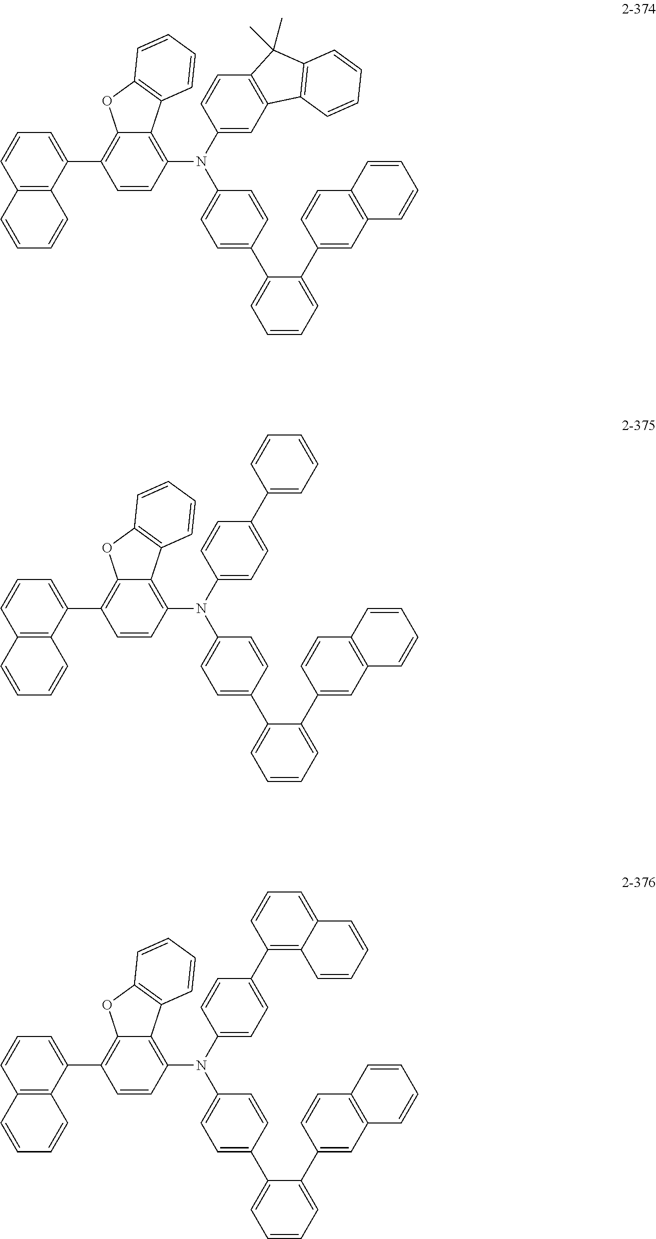 [SMILES: CC1(C)c2ccccc2-c2cc(N(c3ccc(-c4ccccc4-c4ccc5ccccc5c4)cc3)c3ccc(-c4cccc5ccccc45)c4oc5ccccc5c34)ccc21 ; c1ccc(-c2ccc(N(c3ccc(-c4ccccc4-c4ccc5ccccc5c4)cc3)c3ccc(-c4cccc5ccccc45)c4oc5ccccc5c34)cc2)cc1 ; c1ccc(-c2ccc3ccccc3c2)c(-c2ccc(N(c3ccc(-c4cccc5ccccc45)cc3)c3ccc(-c4cccc5ccccc45)c4oc5ccccc5c34)cc2)c1]

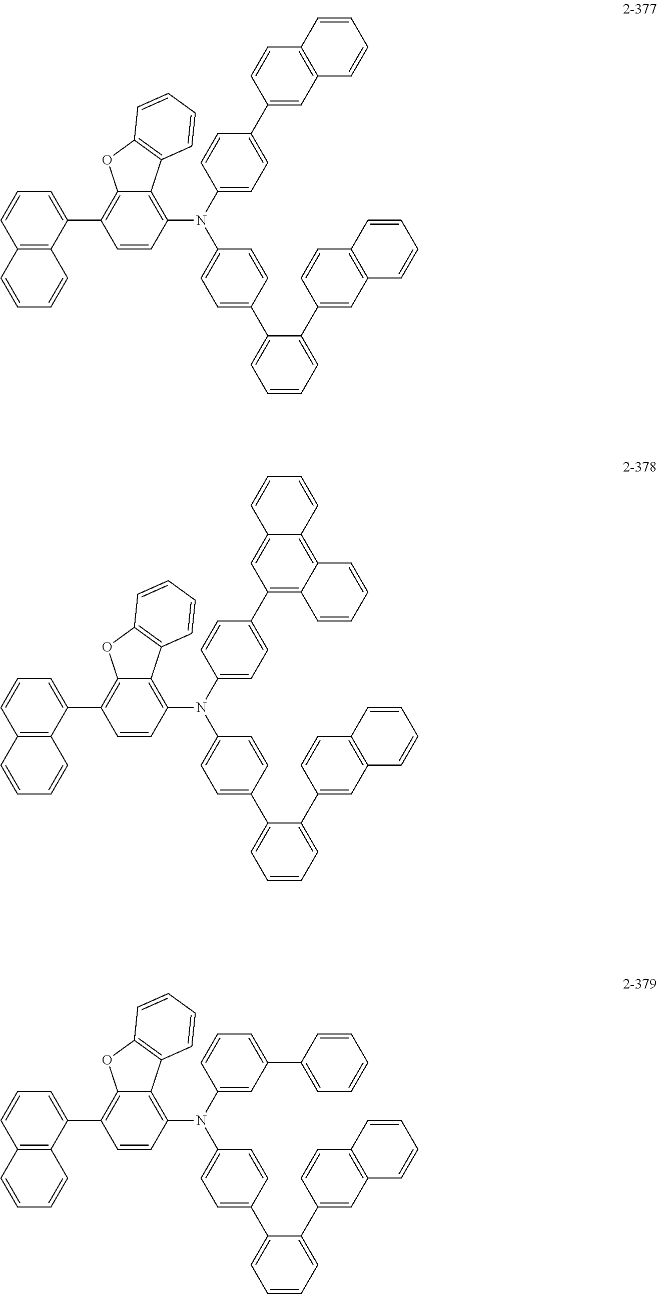 [SMILES: c1ccc(-c2ccc3ccccc3c2)c(-c2ccc(N(c3ccc(-c4ccc5ccccc5c4)cc3)c3ccc(-c4cccc5ccccc45)c4oc5ccccc5c34)cc2)c1 ; c1ccc(-c2ccc3ccccc3c2)c(-c2ccc(N(c3ccc(-c4cc5ccccc5c5ccccc45)cc3)c3ccc(-c4cccc5ccccc45)c4oc5ccccc5c34)cc2)c1 ; c1ccc(-c2cccc(N(c3ccc(-c4ccccc4-c4ccc5ccccc5c4)cc3)c3ccc(-c4cccc5ccccc45)c4oc5ccccc5c34)c2)cc1]

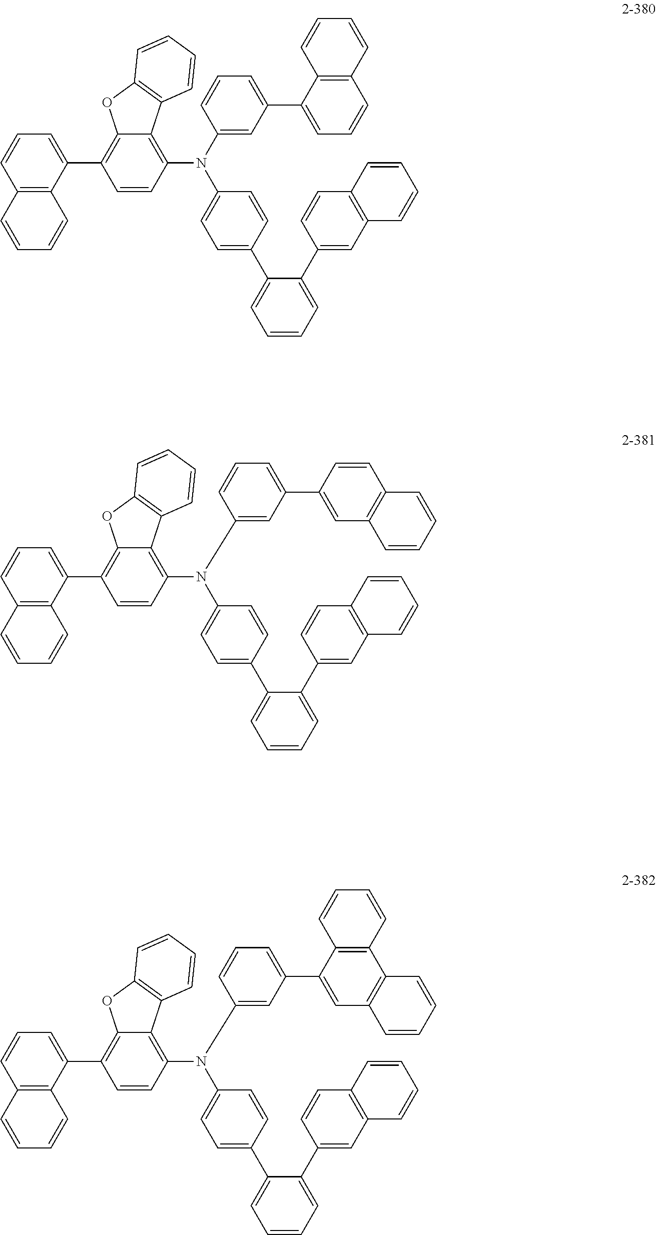 [SMILES: c1cc(-c2cccc3ccccc23)cc(N(c2ccc(-c3ccccc3-c3ccc4ccccc4c3)cc2)c2ccc(-c3cccc4ccccc34)c3oc4ccccc4c23)c1 ; c1cc(-c2ccc3ccccc3c2)cc(N(c2ccc(-c3ccccc3-c3ccc4ccccc4c3)cc2)c2ccc(-c3cccc4ccccc34)c3oc4ccccc4c23)c1 ; c1cc(-c2cc3ccccc3c3ccccc23)cc(N(c2ccc(-c3ccccc3-c3ccc4ccccc4c3)cc2)c2ccc(-c3cccc4ccccc34)c3oc4ccccc4c23)c1]

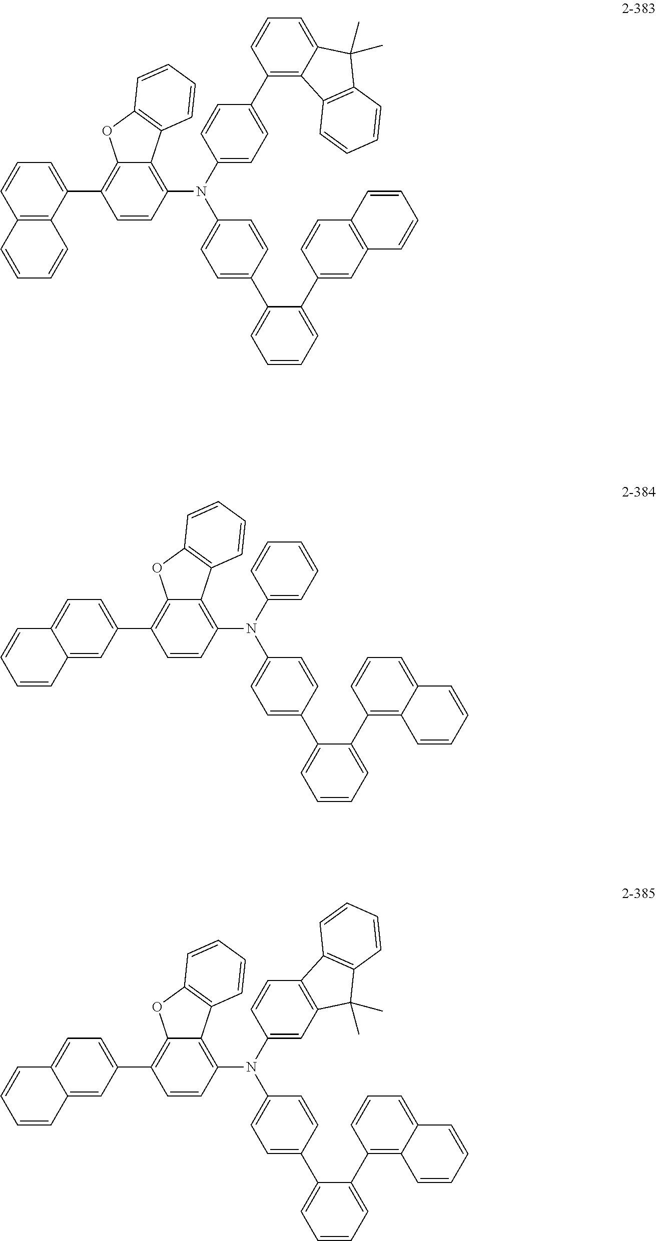 [SMILES: CC1(C)c2ccccc2-c2c(-c3ccc(N(c4ccc(-c5ccccc5-c5ccc6ccccc6c5)cc4)c4ccc(-c5cccc6ccccc56)c5oc6ccccc6c45)cc3)cccc21 ; c1ccc(N(c2ccc(-c3ccccc3-c3cccc4ccccc34)cc2)c2ccc(-c3ccc4ccccc4c3)c3oc4ccccc4c23)cc1 ; CC1(C)c2ccccc2-c2ccc(N(c3ccc(-c4ccccc4-c4cccc5ccccc45)cc3)c3ccc(-c4ccc5ccccc5c4)c4oc5ccccc5c34)cc21]

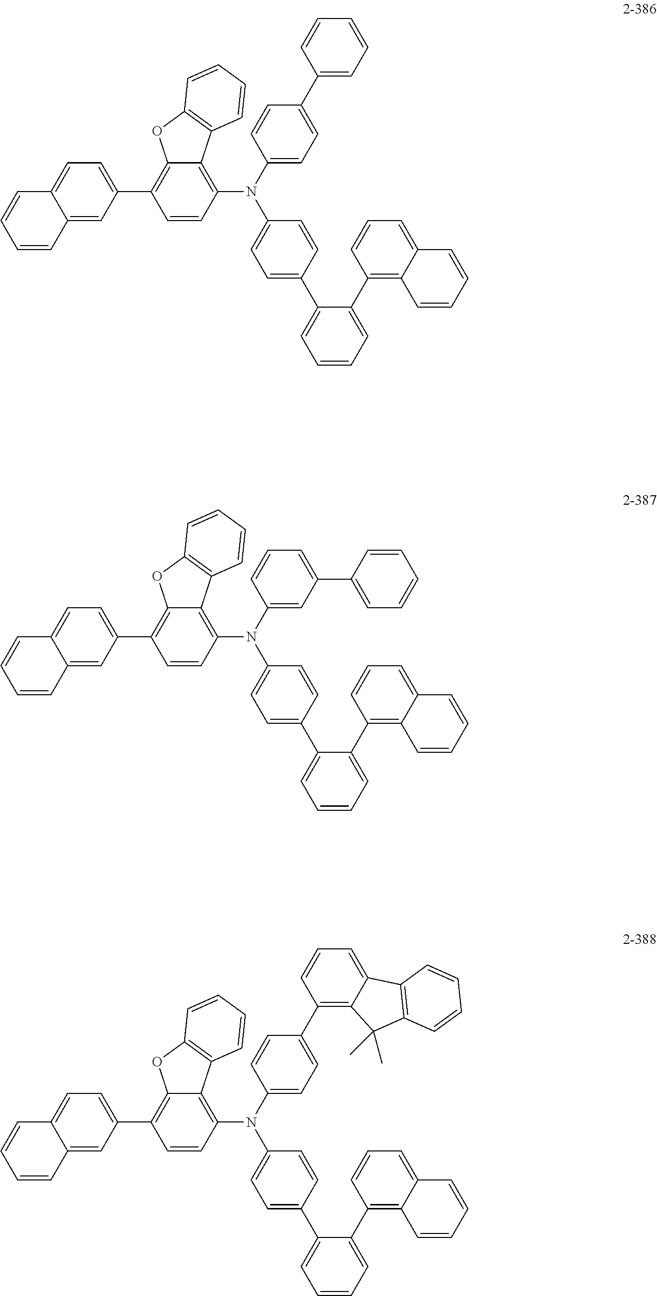 [SMILES: c1ccc(-c2ccc(N(c3ccc(-c4ccccc4-c4cccc5ccccc45)cc3)c3ccc(-c4ccc5ccccc5c4)c4oc5ccccc5c34)cc2)cc1 ; c1ccc(-c2cccc(N(c3ccc(-c4ccccc4-c4cccc5ccccc45)cc3)c3ccc(-c4ccc5ccccc5c4)c4oc5ccccc5c34)c2)cc1 ; CC1(C)c2ccccc2-c2cccc(-c3ccc(N(c4ccc(-c5ccccc5-c5cccc6ccccc56)cc4)c4ccc(-c5ccc6ccccc6c5)c5oc6ccccc6c45)cc3)c21]

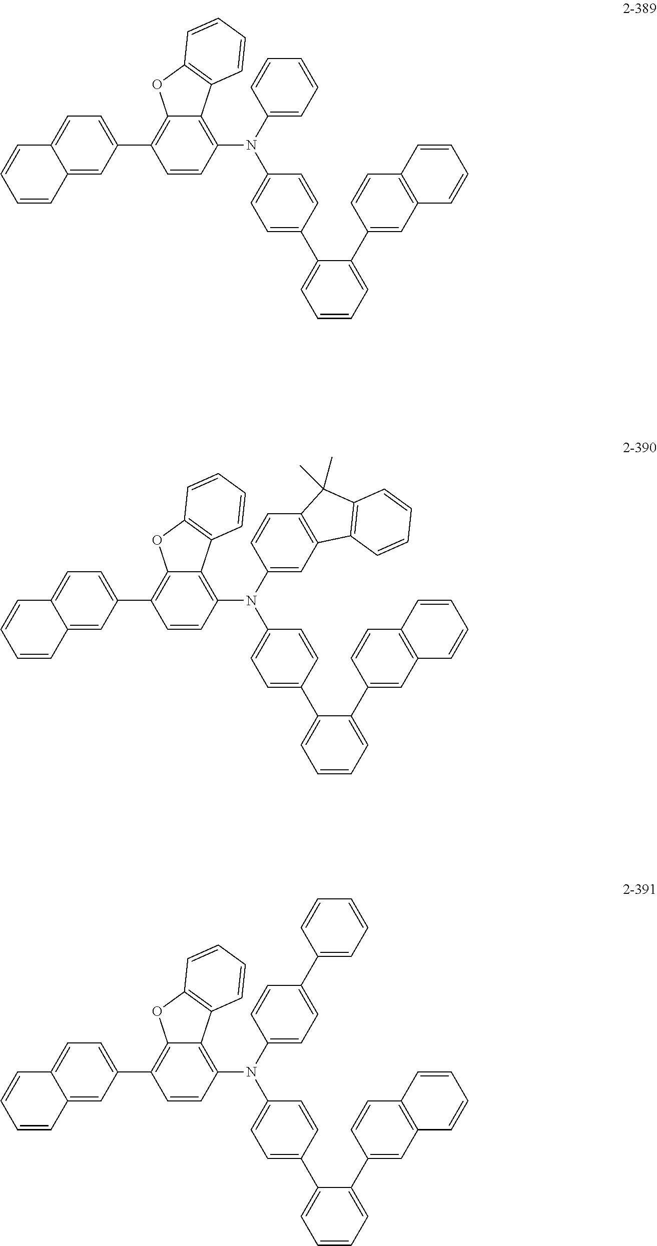 [SMILES: c1ccc(N(c2ccc(-c3ccccc3-c3ccc4ccccc4c3)cc2)c2ccc(-c3ccc4ccccc4c3)c3oc4ccccc4c23)cc1 ; CC1(C)c2ccccc2-c2cc(N(c3ccc(-c4ccccc4-c4ccc5ccccc5c4)cc3)c3ccc(-c4ccc5ccccc5c4)c4oc5ccccc5c34)ccc21 ; c1ccc(-c2ccc(N(c3ccc(-c4ccccc4-c4ccc5ccccc5c4)cc3)c3ccc(-c4ccc5ccccc5c4)c4oc5ccccc5c34)cc2)cc1]

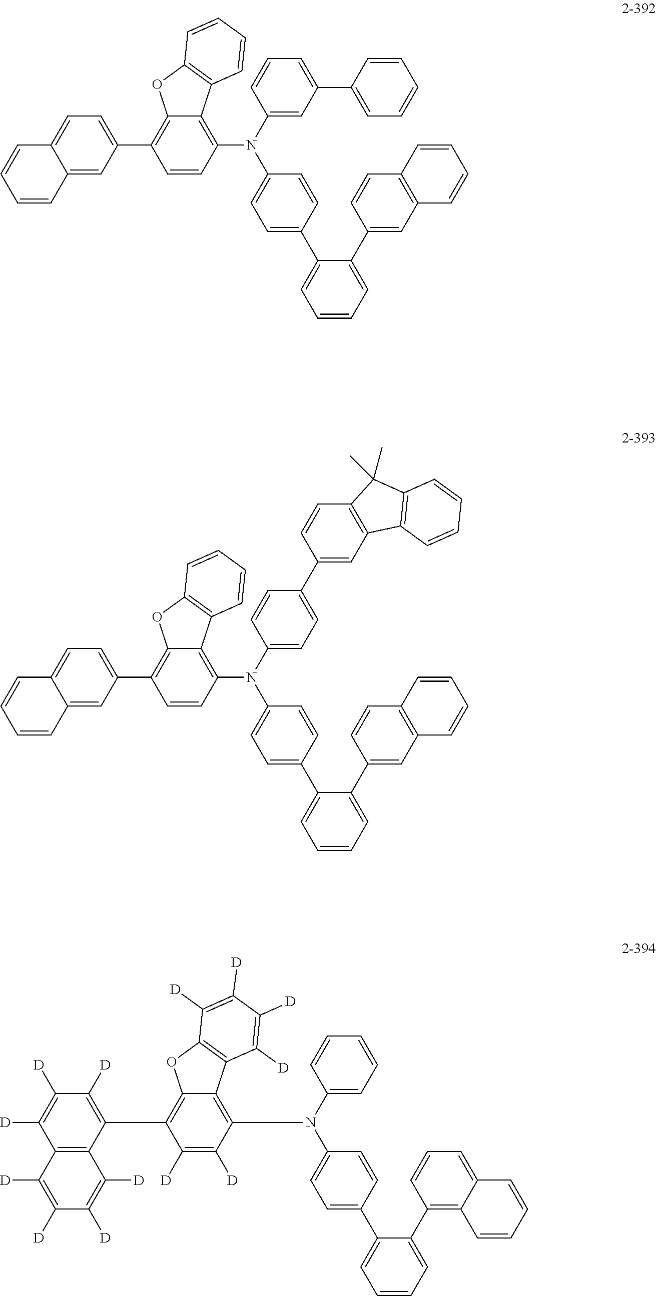 [SMILES: c1ccc(-c2cccc(N(c3ccc(-c4ccccc4-c4ccc5ccccc5c4)cc3)c3ccc(-c4ccc5ccccc5c4)c4oc5ccccc5c34)c2)cc1 ; CC1(C)c2ccccc2-c2cc(-c3ccc(N(c4ccc(-c5ccccc5-c5ccc6ccccc6c5)cc4)c4ccc(-c5ccc6ccccc6c5)c5oc6ccccc6c45)cc3)ccc21 ; [2H]c1c([2H])c([2H])c2c(oc3c(-c4c([2H])c([2H])c([2H])c5c([2H])c([2H])c([2H])c([2H])c45)c([2H])c([2H])c(N(c4ccccc4)c4ccc(-c5ccccc5-c5cccc6ccccc56)cc4)c32)c1[2H]]

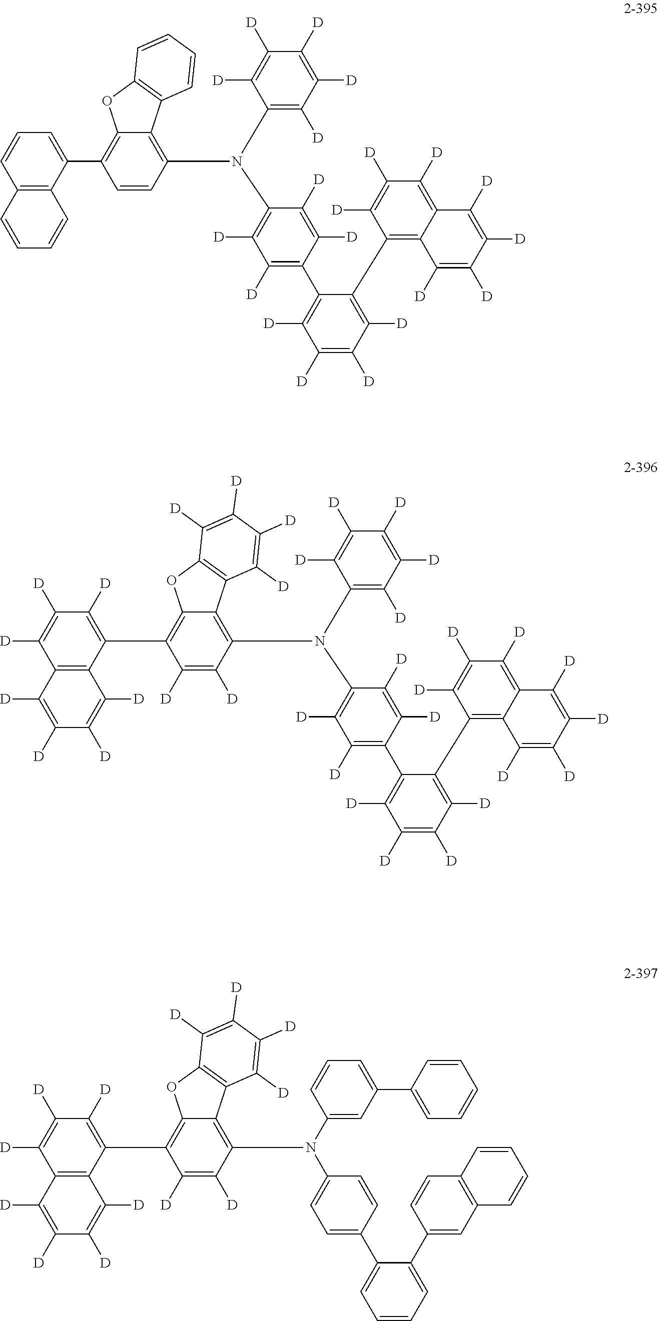

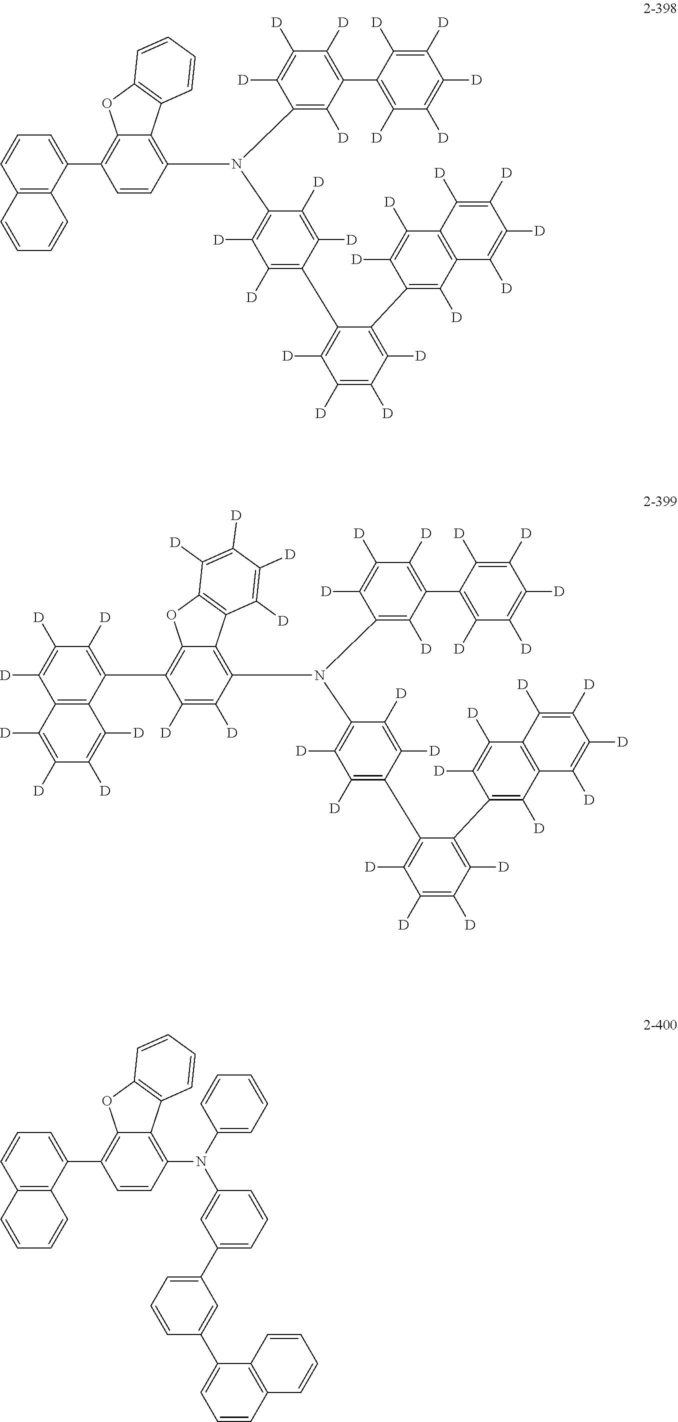

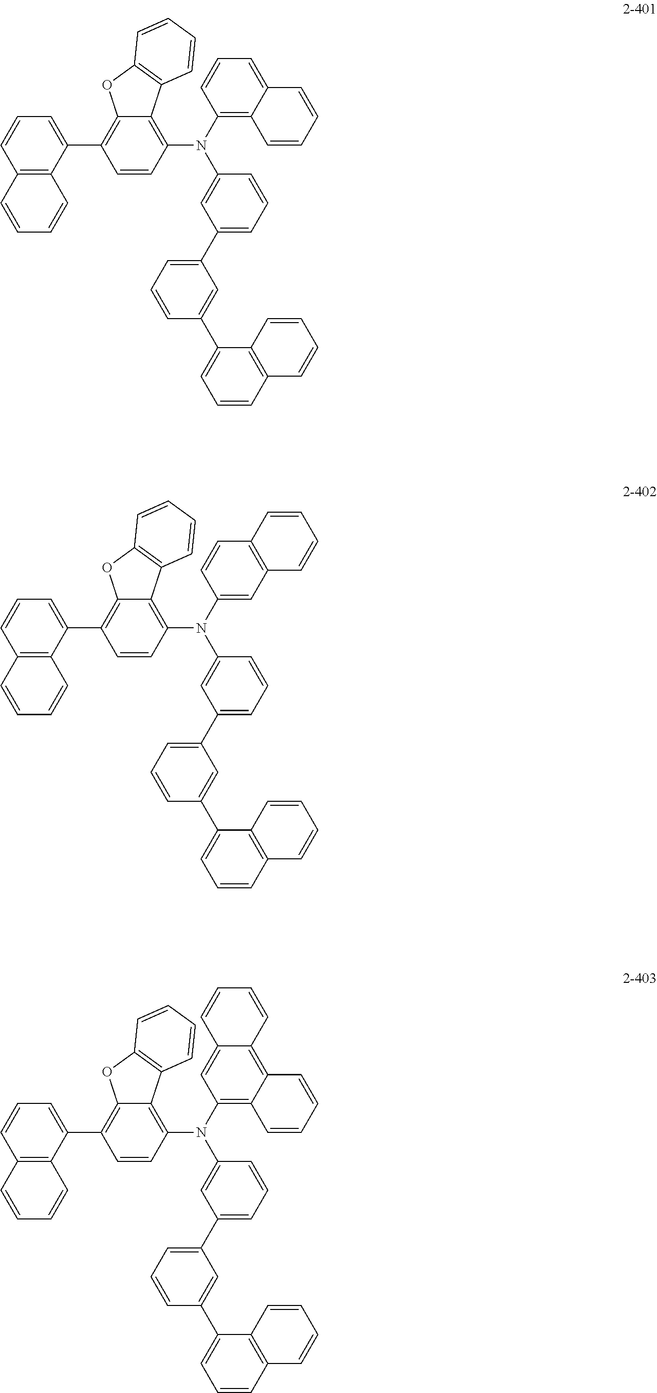 [SMILES: c1cc(-c2cccc(N(c3cccc4ccccc34)c3ccc(-c4cccc5ccccc45)c4oc5ccccc5c34)c2)cc(-c2cccc3ccccc23)c1 ; c1cc(-c2cccc(N(c3ccc4ccccc4c3)c3ccc(-c4cccc5ccccc45)c4oc5ccccc5c34)c2)cc(-c2cccc3ccccc23)c1 ; c1cc(-c2cccc(N(c3cc4ccccc4c4ccccc34)c3ccc(-c4cccc5ccccc45)c4oc5ccccc5c34)c2)cc(-c2cccc3ccccc23)c1]

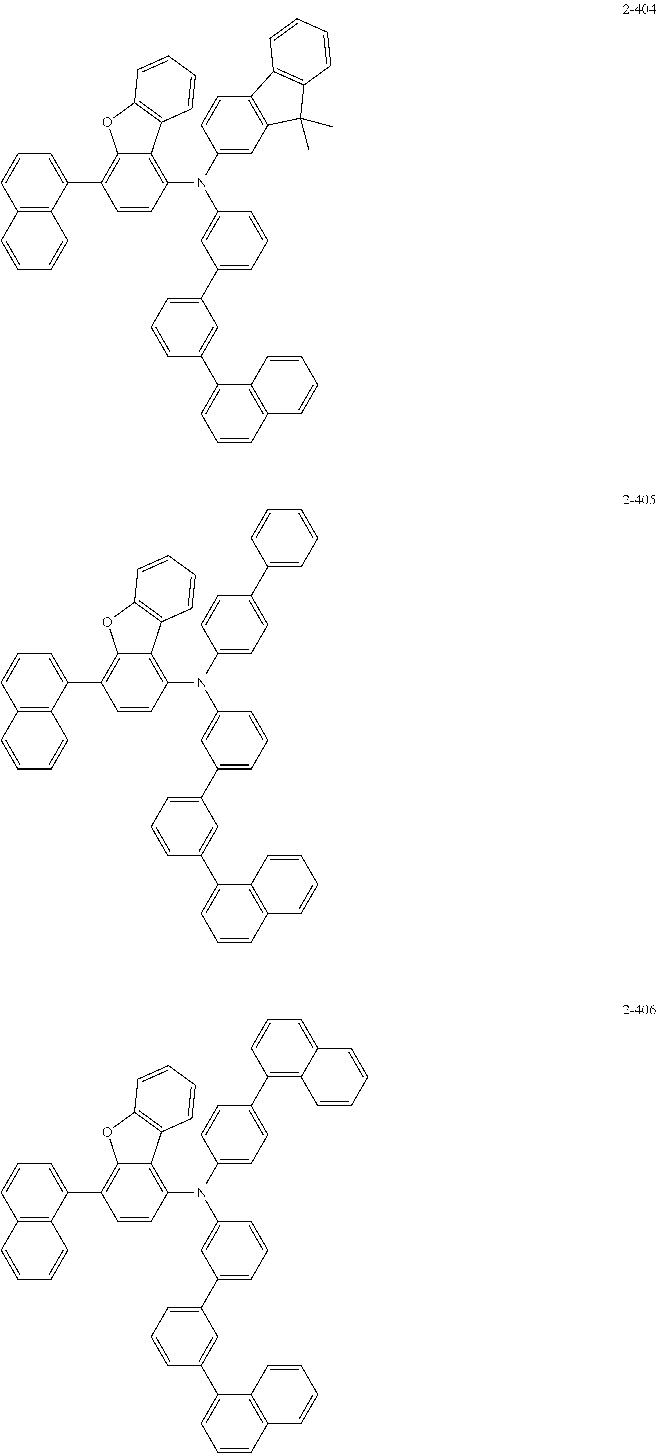 [SMILES: CC1(C)c2ccccc2-c2ccc(N(c3cccc(-c4cccc(-c5cccc6ccccc56)c4)c3)c3ccc(-c4cccc5ccccc45)c4oc5ccccc5c34)cc21 ; c1ccc(-c2ccc(N(c3cccc(-c4cccc(-c5cccc6ccccc56)c4)c3)c3ccc(-c4cccc5ccccc45)c4oc5ccccc5c34)cc2)cc1 ; c1cc(-c2cccc(N(c3ccc(-c4cccc5ccccc45)cc3)c3ccc(-c4cccc5ccccc45)c4oc5ccccc5c34)c2)cc(-c2cccc3ccccc23)c1]

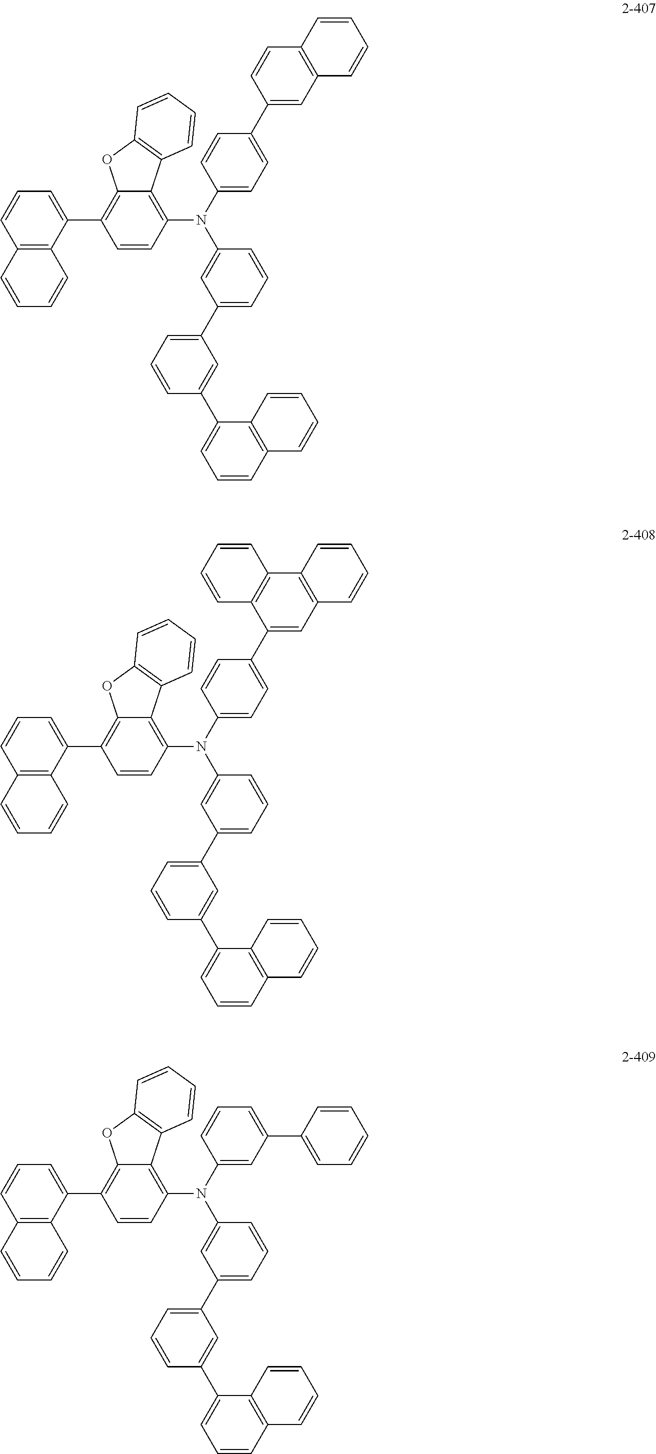 [SMILES: c1cc(-c2cccc(N(c3ccc(-c4ccc5ccccc5c4)cc3)c3ccc(-c4cccc5ccccc45)c4oc5ccccc5c34)c2)cc(-c2cccc3ccccc23)c1 ; c1cc(-c2cccc(N(c3ccc(-c4cc5ccccc5c5ccccc45)cc3)c3ccc(-c4cccc5ccccc45)c4oc5ccccc5c34)c2)cc(-c2cccc3ccccc23)c1 ; c1ccc(-c2cccc(N(c3cccc(-c4cccc(-c5cccc6ccccc56)c4)c3)c3ccc(-c4cccc5ccccc45)c4oc5ccccc5c34)c2)cc1]

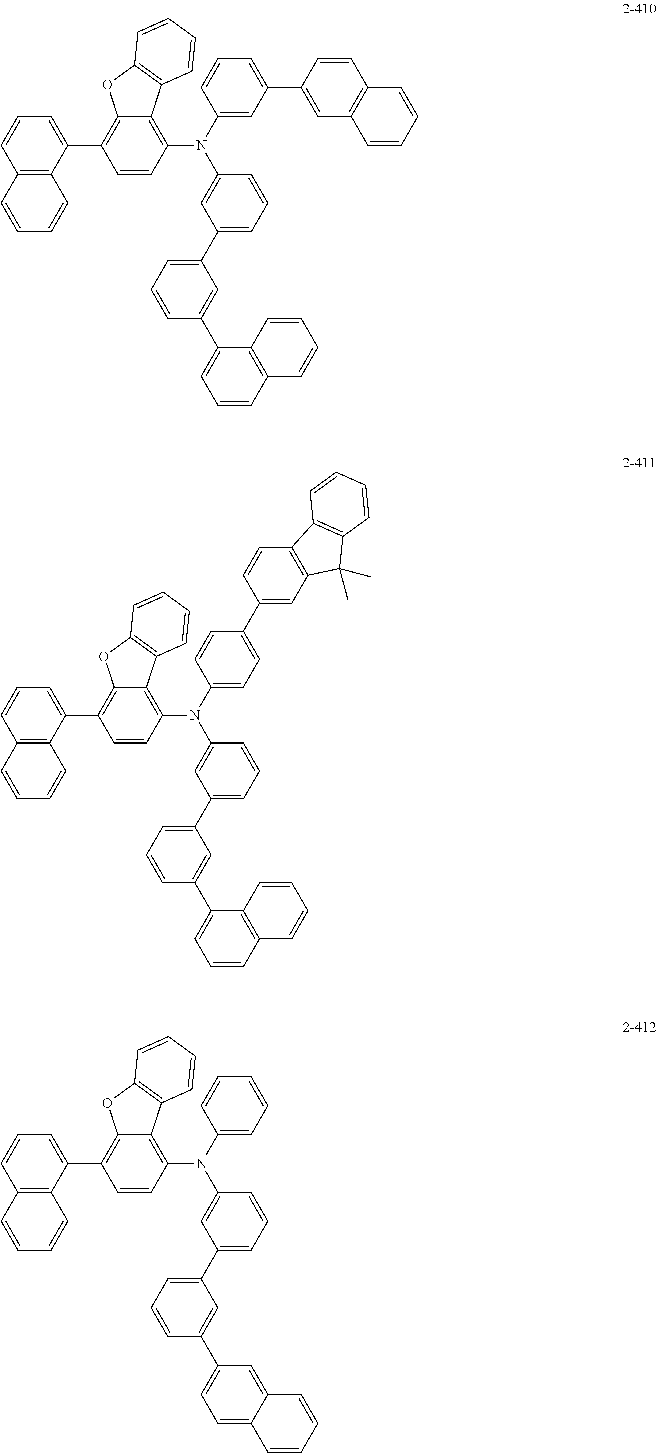 [SMILES: c1cc(-c2cccc(N(c3cccc(-c4ccc5ccccc5c4)c3)c3ccc(-c4cccc5ccccc45)c4oc5ccccc5c34)c2)cc(-c2cccc3ccccc23)c1 ; CC1(C)c2ccccc2-c2ccc(-c3ccc(N(c4cccc(-c5cccc(-c6cccc7ccccc67)c5)c4)c4ccc(-c5cccc6ccccc56)c5oc6ccccc6c45)cc3)cc21 ; c1ccc(N(c2cccc(-c3cccc(-c4ccc5ccccc5c4)c3)c2)c2ccc(-c3cccc4ccccc34)c3oc4ccccc4c23)cc1]

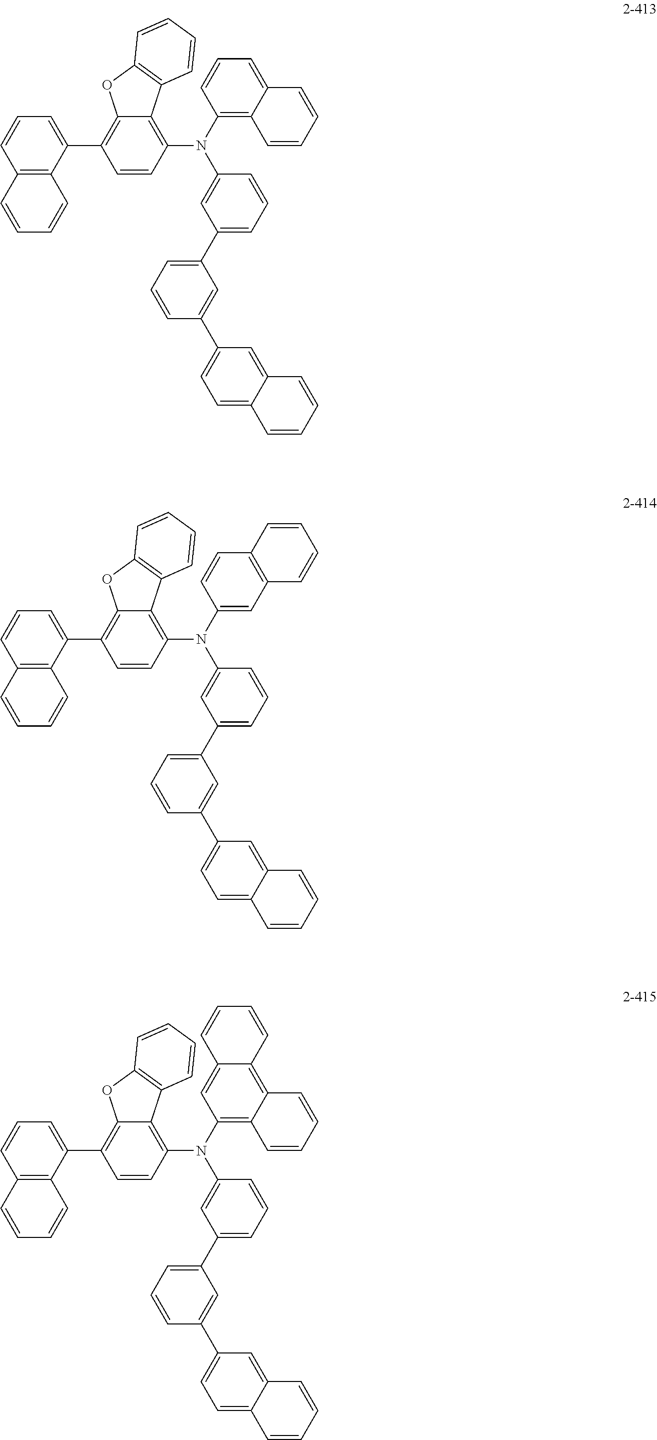 [SMILES: c1cc(-c2cccc(N(c3cccc4ccccc34)c3ccc(-c4cccc5ccccc45)c4oc5ccccc5c34)c2)cc(-c2ccc3ccccc3c2)c1 ; c1cc(-c2cccc(N(c3ccc4ccccc4c3)c3ccc(-c4cccc5ccccc45)c4oc5ccccc5c34)c2)cc(-c2ccc3ccccc3c2)c1 ; c1cc(-c2cccc(N(c3cc4ccccc4c4ccccc34)c3ccc(-c4cccc5ccccc45)c4oc5ccccc5c34)c2)cc(-c2ccc3ccccc3c2)c1]

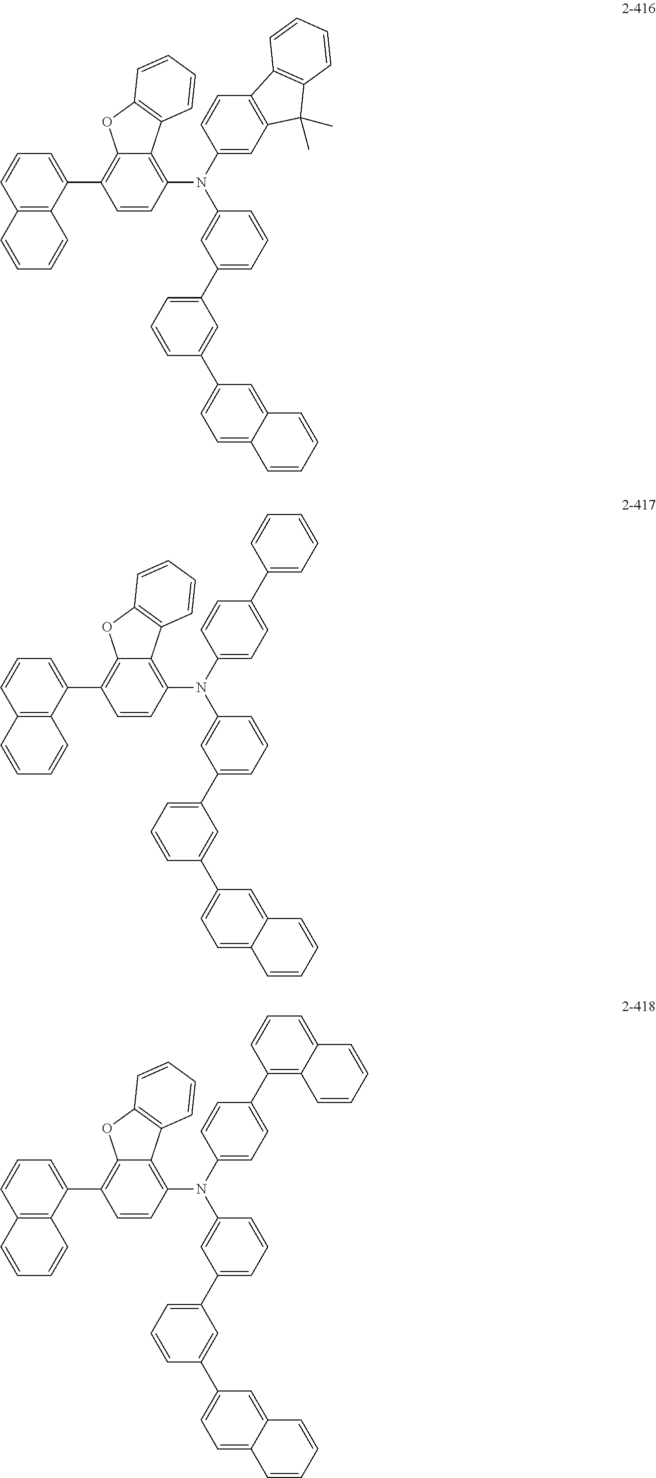

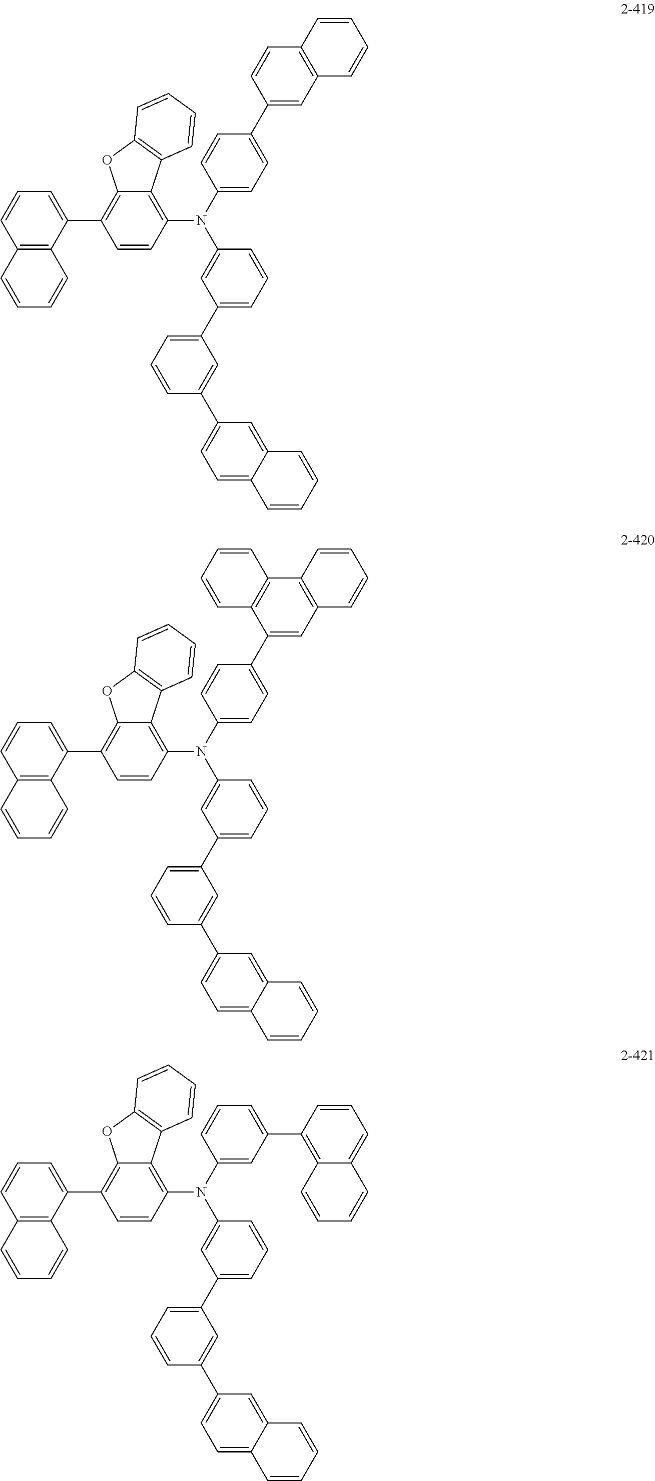

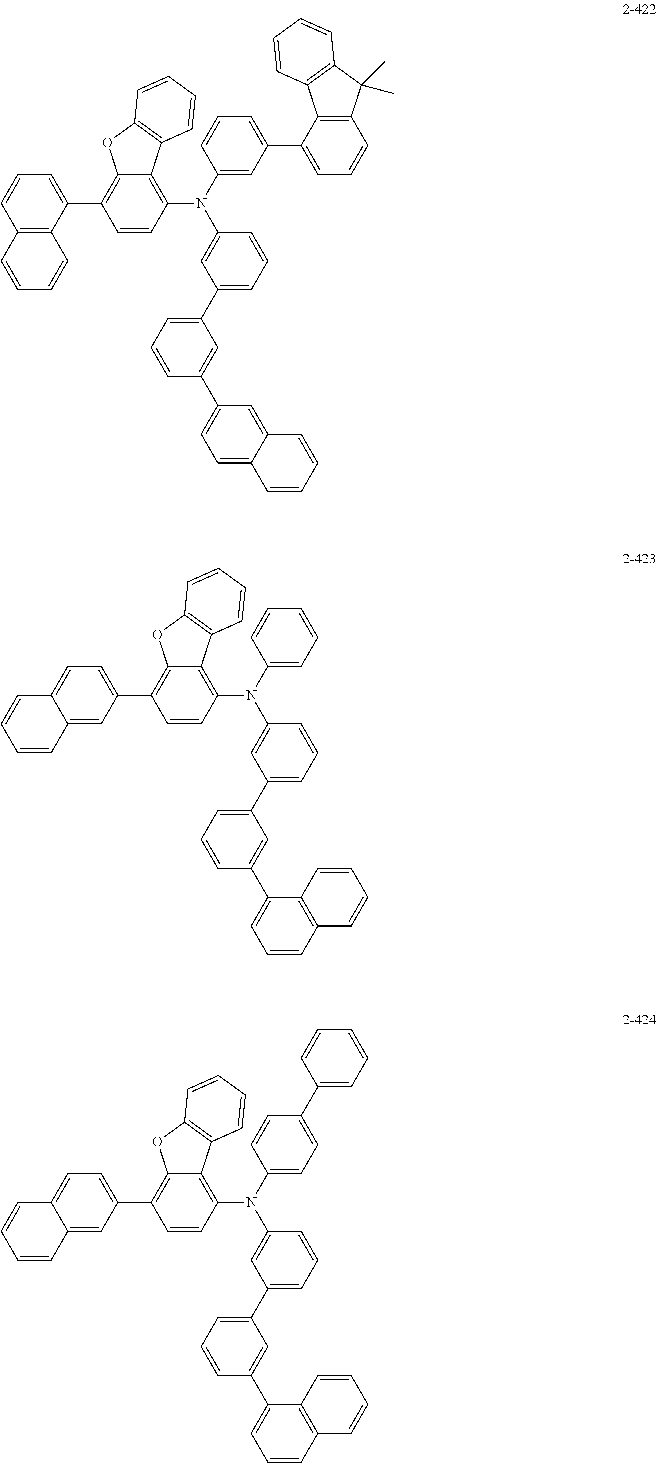 [SMILES: CC1(C)c2ccccc2-c2c(-c3cccc(N(c4cccc(-c5cccc(-c6ccc7ccccc7c6)c5)c4)c4ccc(-c5cccc6ccccc56)c5oc6ccccc6c45)c3)cccc21 ; c1ccc(N(c2cccc(-c3cccc(-c4cccc5ccccc45)c3)c2)c2ccc(-c3ccc4ccccc4c3)c3oc4ccccc4c23)cc1 ; c1ccc(-c2ccc(N(c3cccc(-c4cccc(-c5cccc6ccccc56)c4)c3)c3ccc(-c4ccc5ccccc5c4)c4oc5ccccc5c34)cc2)cc1]

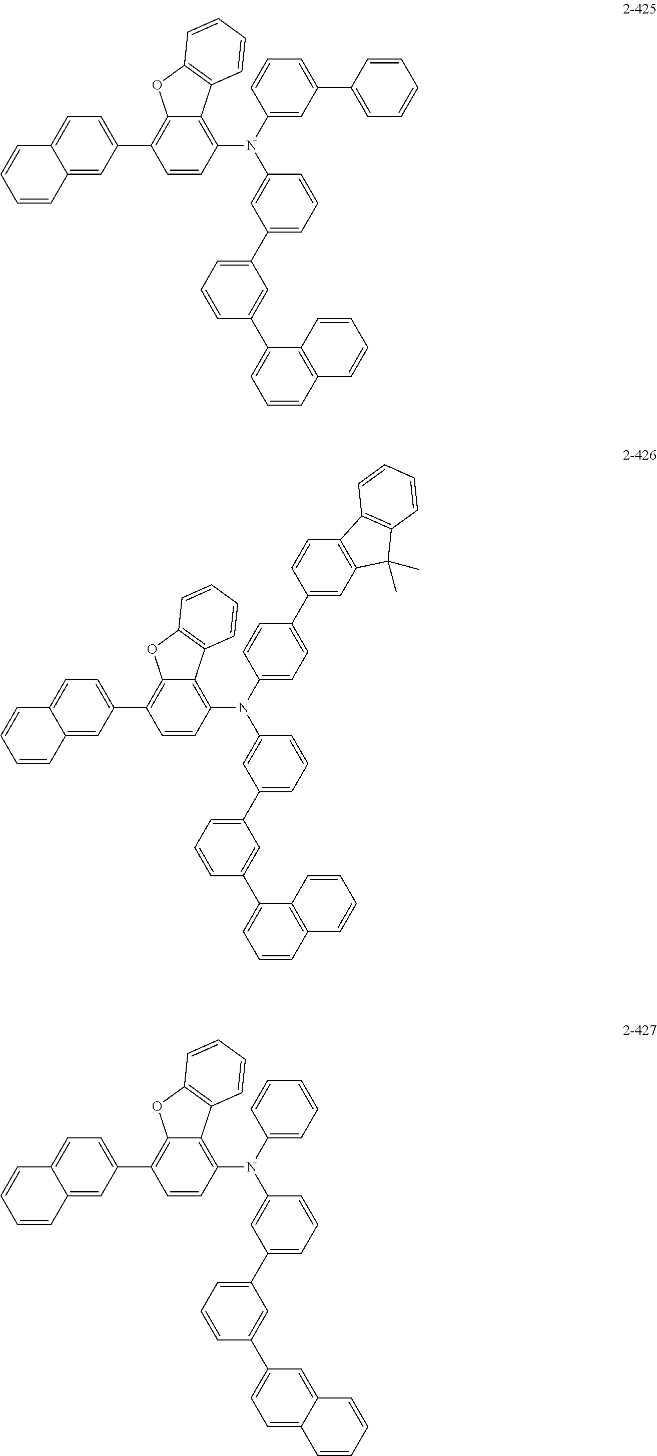 [SMILES: c1ccc(-c2cccc(N(c3cccc(-c4cccc(-c5cccc6ccccc56)c4)c3)c3ccc(-c4ccc5ccccc5c4)c4oc5ccccc5c34)c2)cc1 ; CC1(C)c2ccccc2-c2ccc(-c3ccc(N(c4cccc(-c5cccc(-c6cccc7ccccc67)c5)c4)c4ccc(-c5ccc6ccccc6c5)c5oc6ccccc6c45)cc3)cc21 ; c1ccc(N(c2cccc(-c3cccc(-c4ccc5ccccc5c4)c3)c2)c2ccc(-c3ccc4ccccc4c3)c3oc4ccccc4c23)cc1]

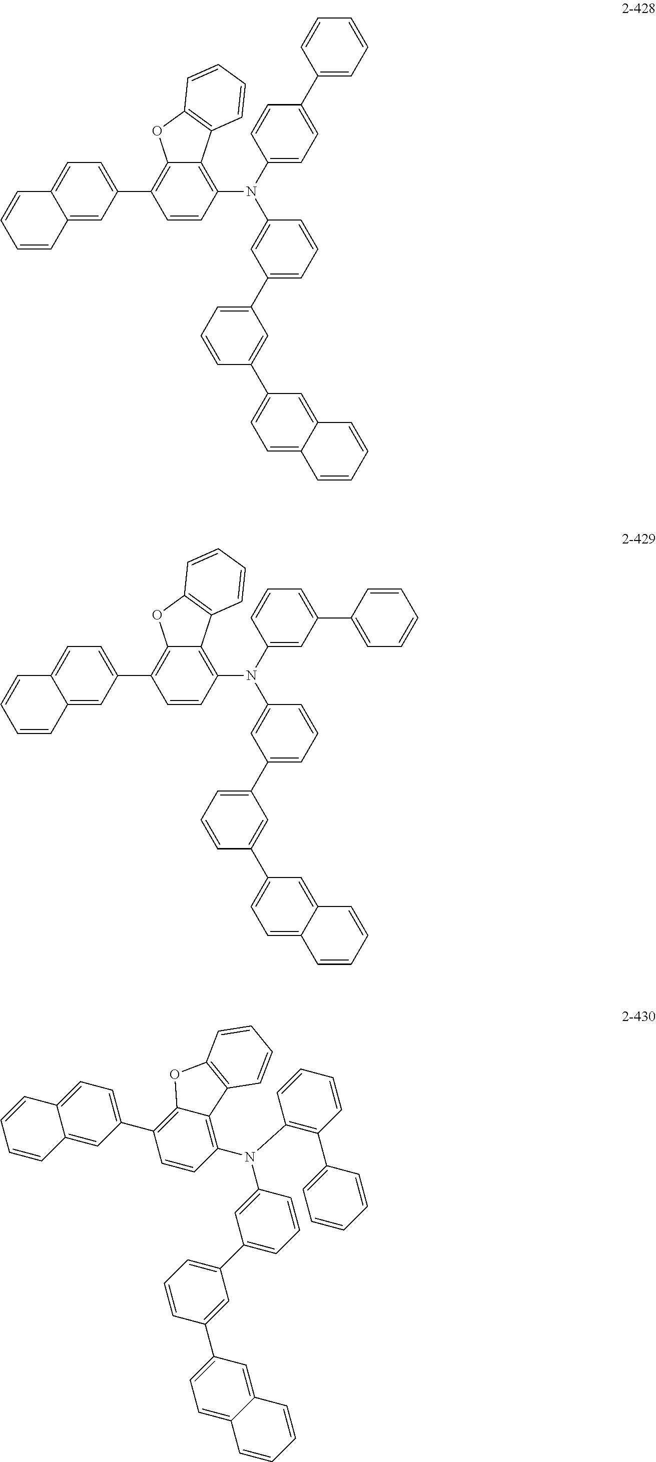 [SMILES: c1ccc(-c2ccc(N(c3cccc(-c4cccc(-c5ccc6ccccc6c5)c4)c3)c3ccc(-c4ccc5ccccc5c4)c4oc5ccccc5c34)cc2)cc1 ; c1ccc(-c2cccc(N(c3cccc(-c4cccc(-c5ccc6ccccc6c5)c4)c3)c3ccc(-c4ccc5ccccc5c4)c4oc5ccccc5c34)c2)cc1 ; c1ccc(-c2ccccc2N(c2cccc(-c3cccc(-c4ccc5ccccc5c4)c3)c2)c2ccc(-c3ccc4ccccc4c3)c3oc4ccccc4c23)cc1]

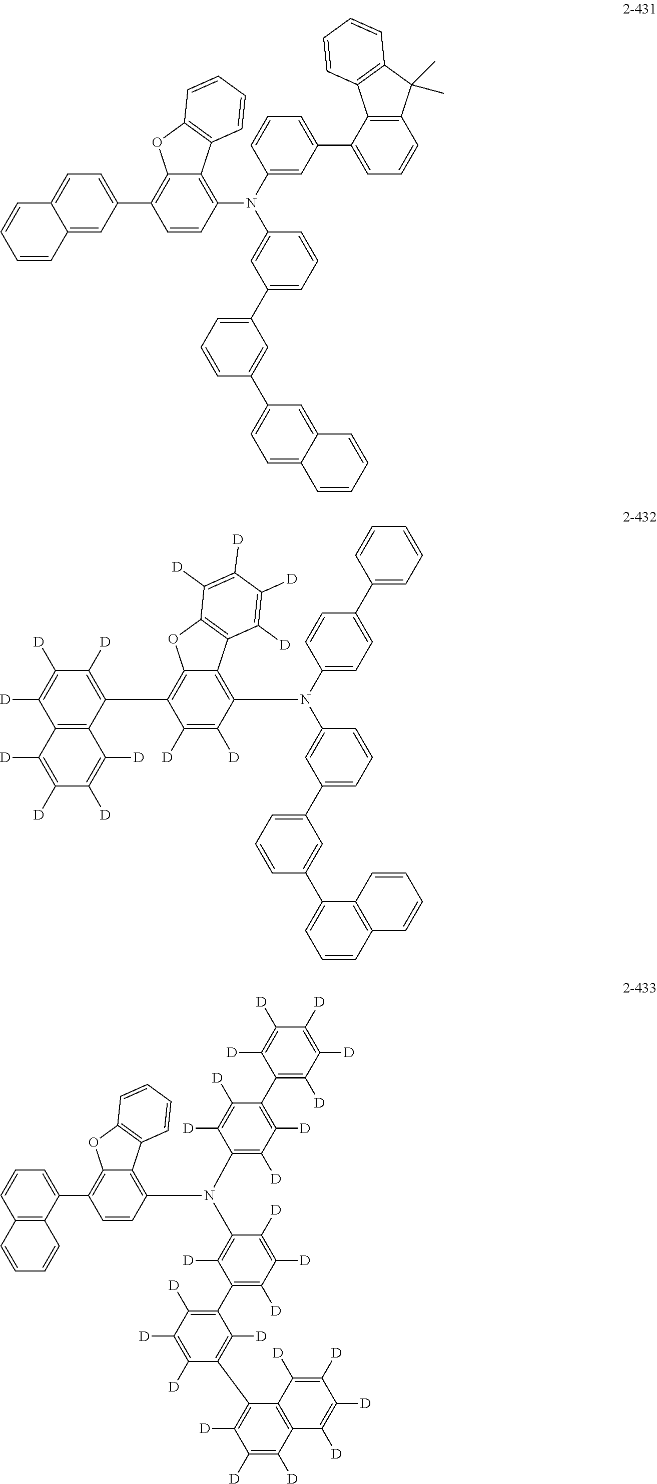

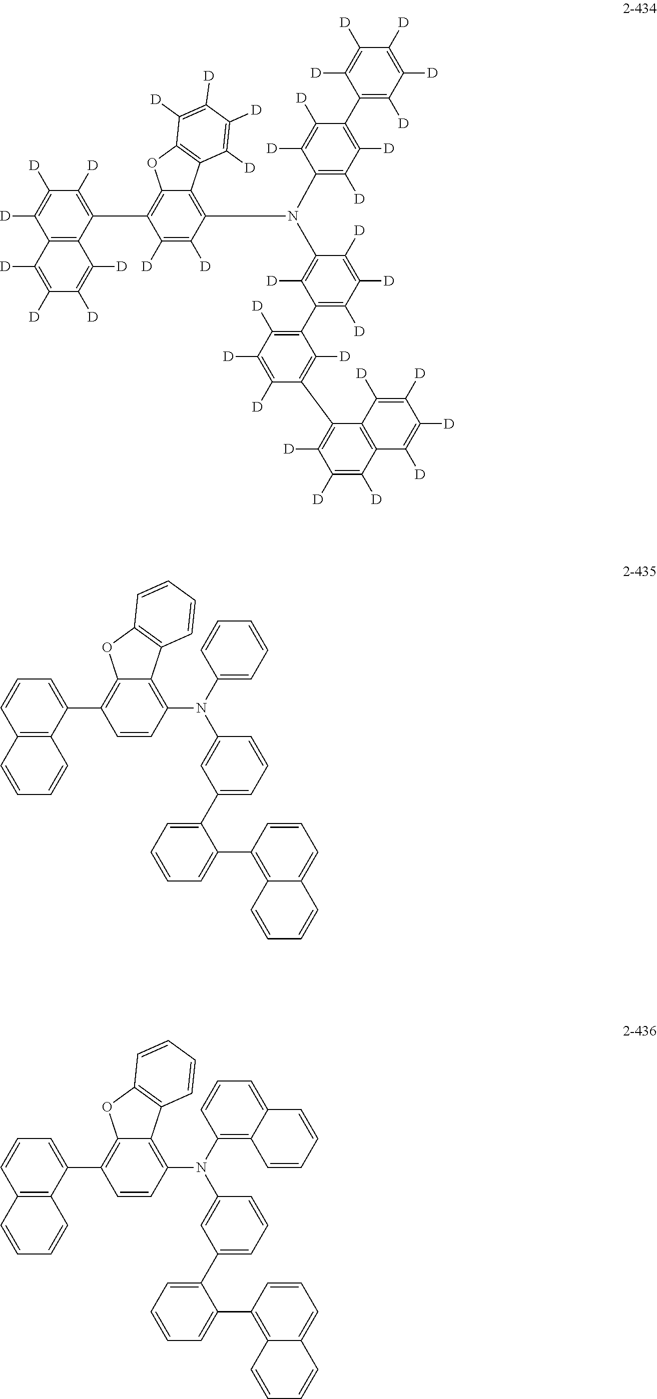 [SMILES: [2H]c1c([2H])c([2H])c(-c2c([2H])c([2H])c(N(c3c([2H])c([2H])c([2H])c(-c4c([2H])c([2H])c([2H])c(-c5c([2H])c([2H])c([2H])c6c([2H])c([2H])c([2H])c([2H])c56)c4[2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H] ; c1ccc(N(c2cccc(-c3ccccc3-c3cccc4ccccc34)c2)c2ccc(-c3cccc4ccccc34)c3oc4ccccc4c23)cc1 ; c1cc(-c2ccccc2-c2cccc3ccccc23)cc(N(c2cccc3ccccc23)c2ccc(-c3cccc4ccccc34)c3oc4ccccc4c23)c1]

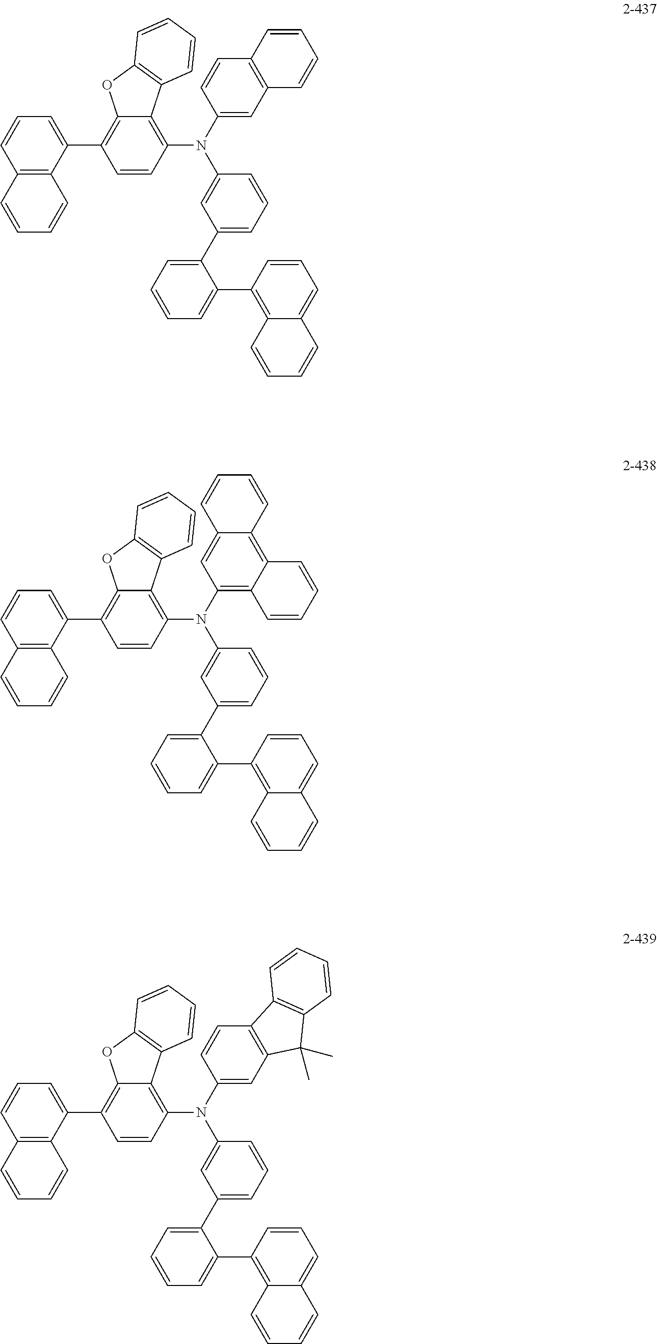 [SMILES: c1cc(-c2ccccc2-c2cccc3ccccc23)cc(N(c2ccc3ccccc3c2)c2ccc(-c3cccc4ccccc34)c3oc4ccccc4c23)c1 ; c1cc(-c2ccccc2-c2cccc3ccccc23)cc(N(c2cc3ccccc3c3ccccc23)c2ccc(-c3cccc4ccccc34)c3oc4ccccc4c23)c1 ; CC1(C)c2ccccc2-c2ccc(N(c3cccc(-c4ccccc4-c4cccc5ccccc45)c3)c3ccc(-c4cccc5ccccc45)c4oc5ccccc5c34)cc21]

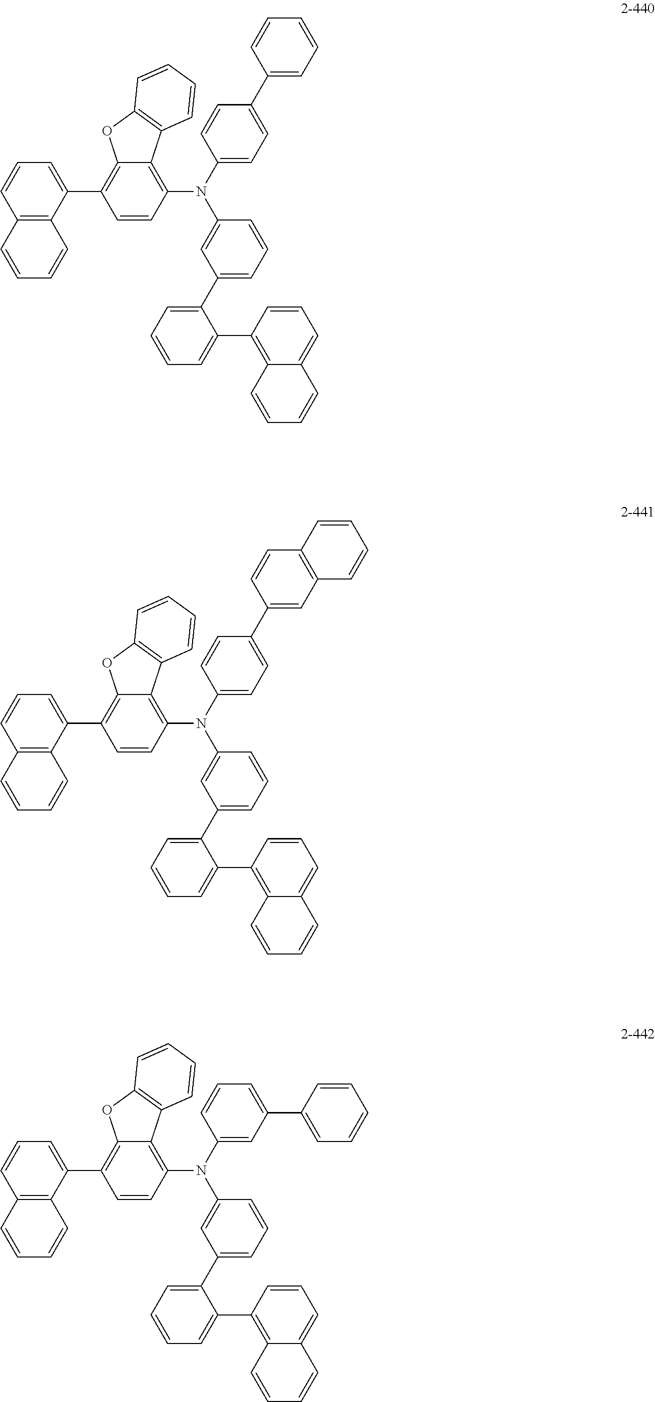 [SMILES: c1ccc(-c2ccc(N(c3cccc(-c4ccccc4-c4cccc5ccccc45)c3)c3ccc(-c4cccc5ccccc45)c4oc5ccccc5c34)cc2)cc1 ; c1cc(-c2ccccc2-c2cccc3ccccc23)cc(N(c2ccc(-c3ccc4ccccc4c3)cc2)c2ccc(-c3cccc4ccccc34)c3oc4ccccc4c23)c1 ; c1ccc(-c2cccc(N(c3cccc(-c4ccccc4-c4cccc5ccccc45)c3)c3ccc(-c4cccc5ccccc45)c4oc5ccccc5c34)c2)cc1]

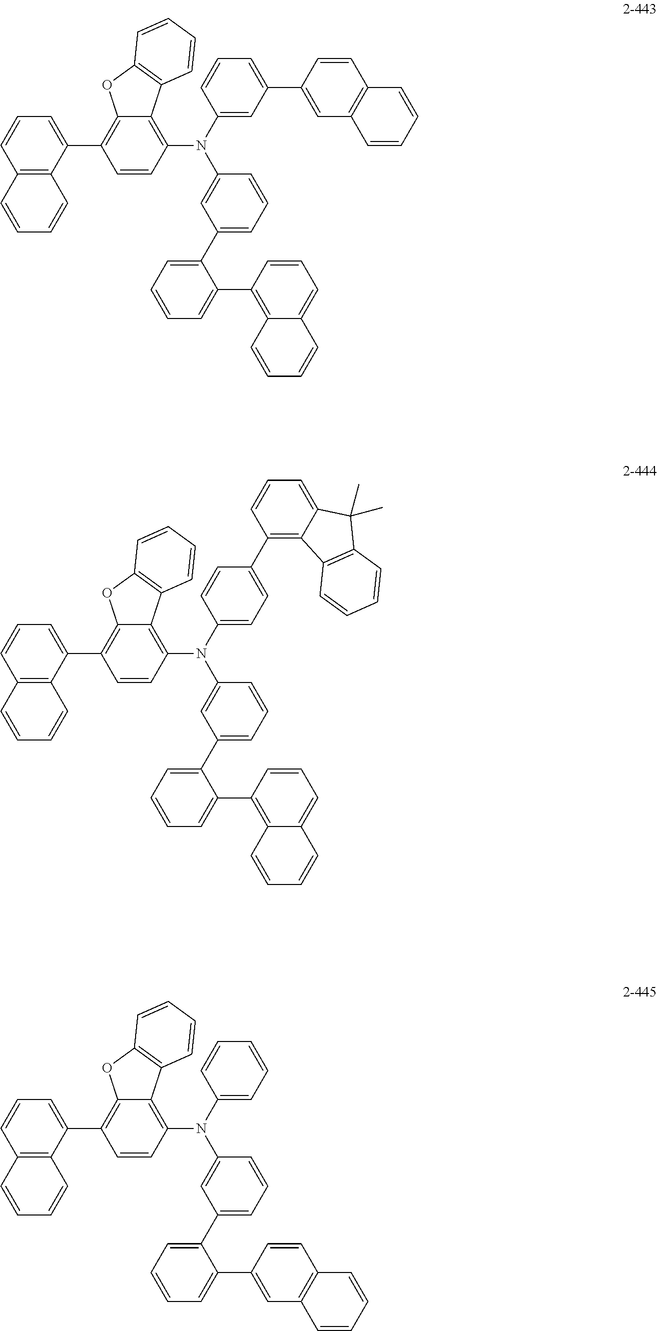 [SMILES: c1cc(-c2ccc3ccccc3c2)cc(N(c2cccc(-c3ccccc3-c3cccc4ccccc34)c2)c2ccc(-c3cccc4ccccc34)c3oc4ccccc4c23)c1 ; CC1(C)c2ccccc2-c2c(-c3ccc(N(c4cccc(-c5ccccc5-c5cccc6ccccc56)c4)c4ccc(-c5cccc6ccccc56)c5oc6ccccc6c45)cc3)cccc21 ; c1ccc(N(c2cccc(-c3ccccc3-c3ccc4ccccc4c3)c2)c2ccc(-c3cccc4ccccc34)c3oc4ccccc4c23)cc1]

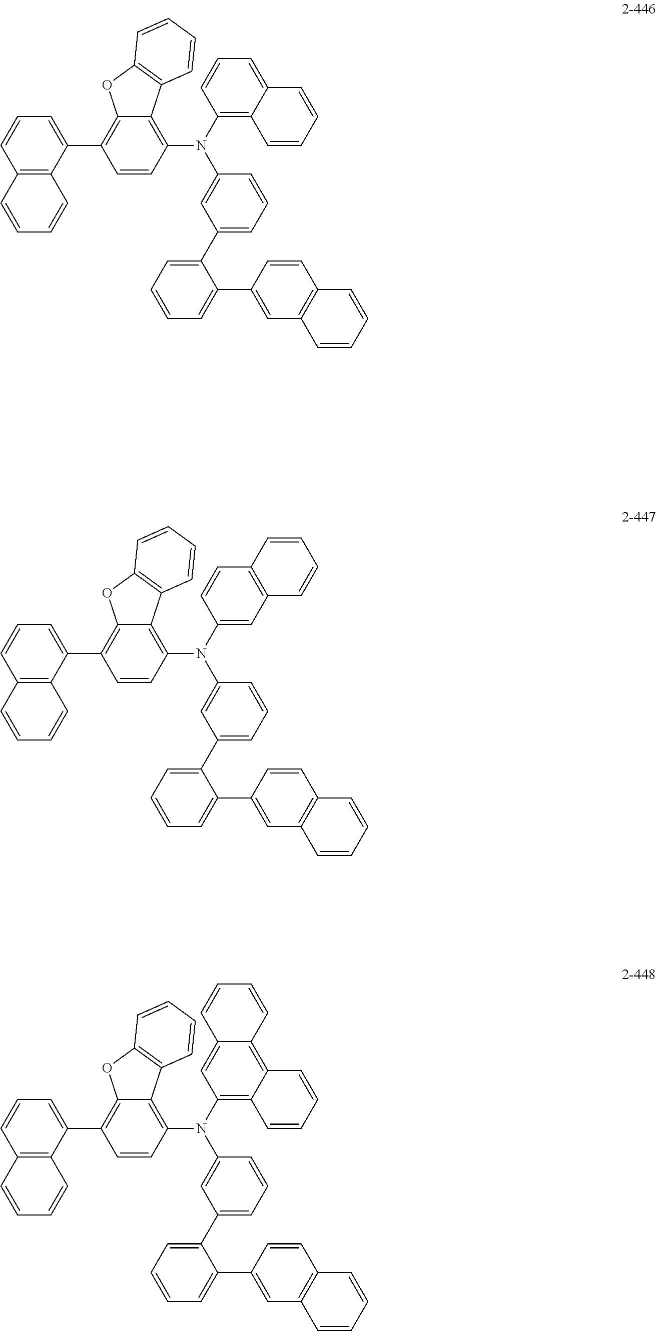 [SMILES: c1cc(-c2ccccc2-c2ccc3ccccc3c2)cc(N(c2cccc3ccccc23)c2ccc(-c3cccc4ccccc34)c3oc4ccccc4c23)c1 ; c1cc(-c2ccccc2-c2ccc3ccccc3c2)cc(N(c2ccc3ccccc3c2)c2ccc(-c3cccc4ccccc34)c3oc4ccccc4c23)c1 ; c1cc(-c2ccccc2-c2ccc3ccccc3c2)cc(N(c2cc3ccccc3c3ccccc23)c2ccc(-c3cccc4ccccc34)c3oc4ccccc4c23)c1]

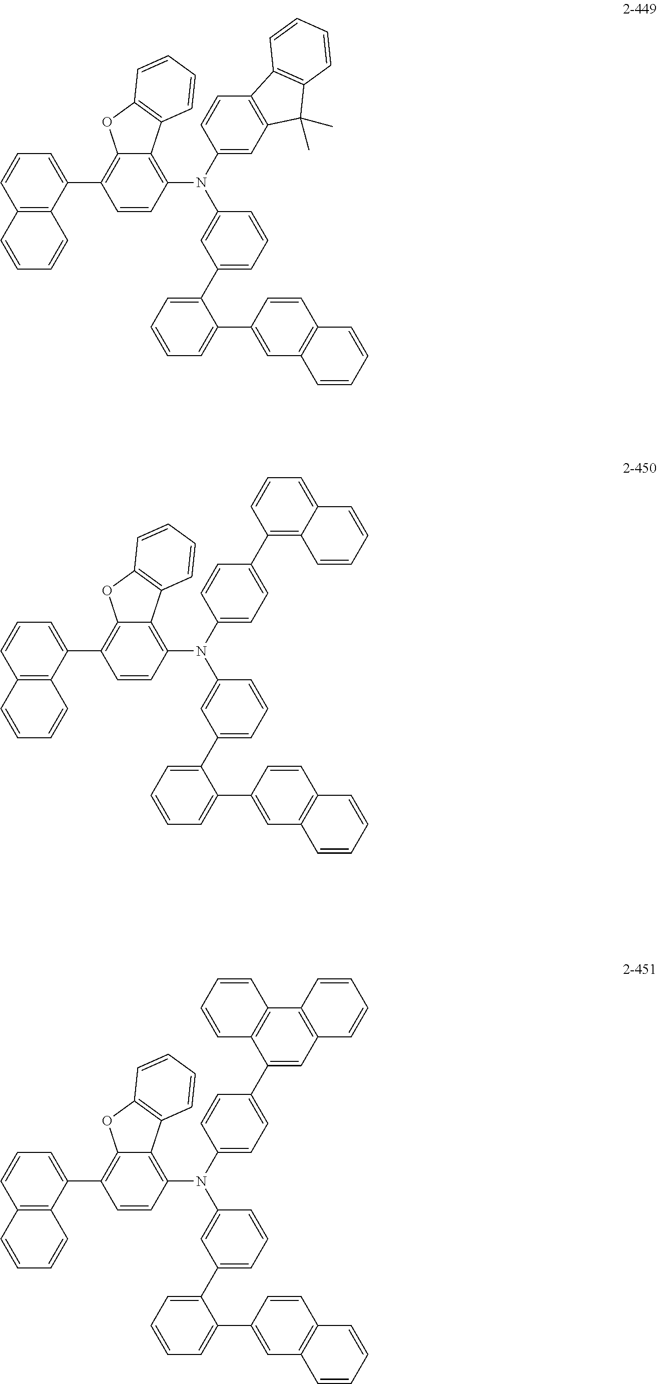 [SMILES: CC1(C)c2ccccc2-c2ccc(N(c3cccc(-c4ccccc4-c4ccc5ccccc5c4)c3)c3ccc(-c4cccc5ccccc45)c4oc5ccccc5c34)cc21 ; c1cc(-c2ccccc2-c2ccc3ccccc3c2)cc(N(c2ccc(-c3cccc4ccccc34)cc2)c2ccc(-c3cccc4ccccc34)c3oc4ccccc4c23)c1 ; c1cc(-c2ccccc2-c2ccc3ccccc3c2)cc(N(c2ccc(-c3cc4ccccc4c4ccccc34)cc2)c2ccc(-c3cccc4ccccc34)c3oc4ccccc4c23)c1]

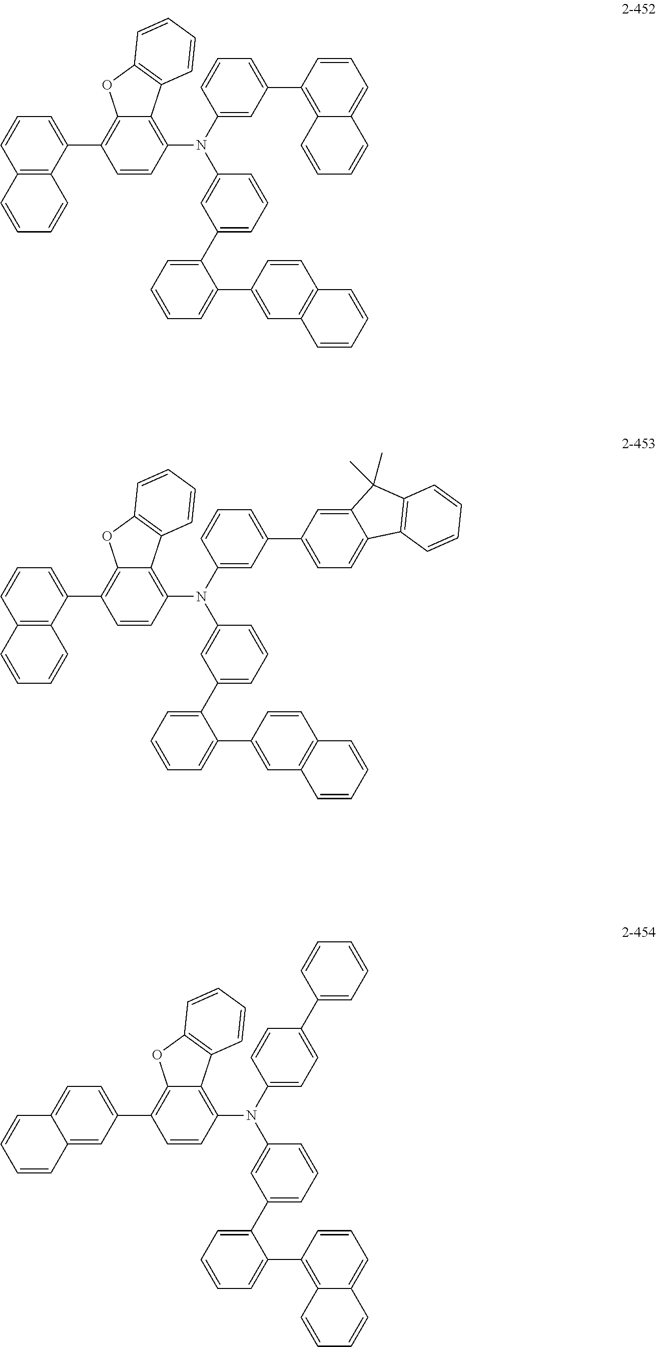 [SMILES: c1cc(-c2ccccc2-c2ccc3ccccc3c2)cc(N(c2cccc(-c3cccc4ccccc34)c2)c2ccc(-c3cccc4ccccc34)c3oc4ccccc4c23)c1 ; CC1(C)c2ccccc2-c2ccc(-c3cccc(N(c4cccc(-c5ccccc5-c5ccc6ccccc6c5)c4)c4ccc(-c5cccc6ccccc56)c5oc6ccccc6c45)c3)cc21 ; c1ccc(-c2ccc(N(c3cccc(-c4ccccc4-c4cccc5ccccc45)c3)c3ccc(-c4ccc5ccccc5c4)c4oc5ccccc5c34)cc2)cc1]

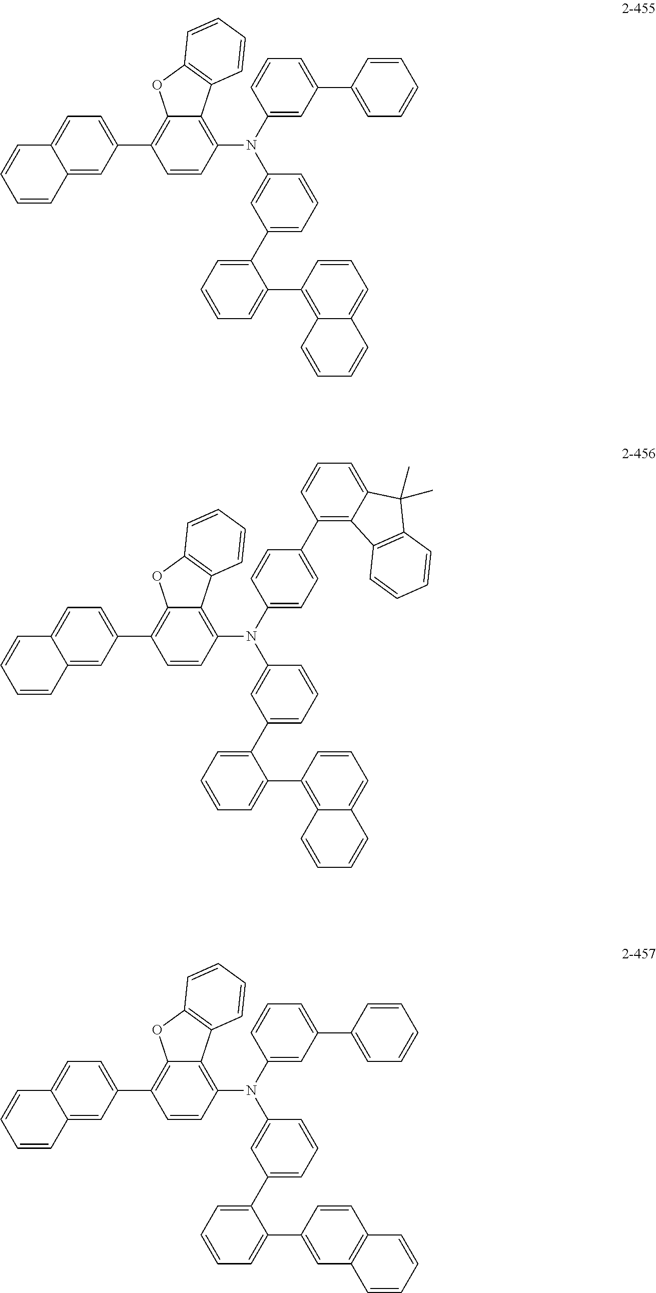 [SMILES: c1ccc(-c2cccc(N(c3cccc(-c4ccccc4-c4cccc5ccccc45)c3)c3ccc(-c4ccc5ccccc5c4)c4oc5ccccc5c34)c2)cc1 ; CC1(C)c2ccccc2-c2c(-c3ccc(N(c4cccc(-c5ccccc5-c5cccc6ccccc56)c4)c4ccc(-c5ccc6ccccc6c5)c5oc6ccccc6c45)cc3)cccc21 ; c1ccc(-c2cccc(N(c3cccc(-c4ccccc4-c4ccc5ccccc5c4)c3)c3ccc(-c4ccc5ccccc5c4)c4oc5ccccc5c34)c2)cc1]

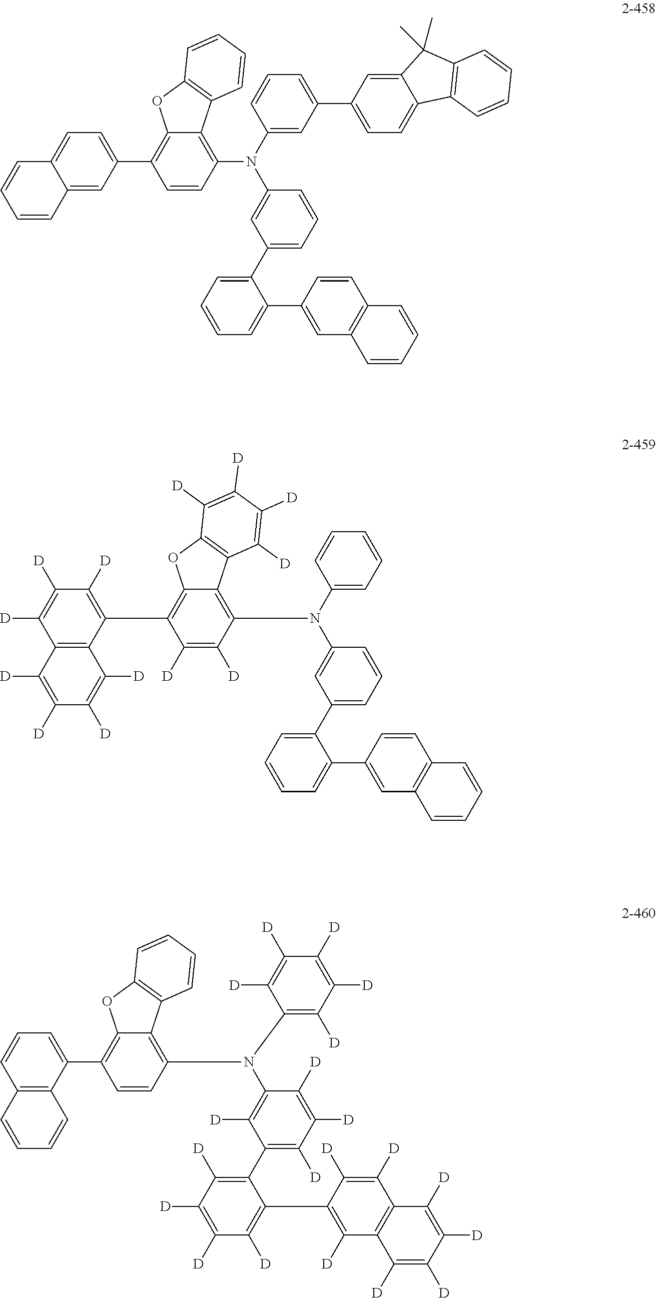 [SMILES: CC1(C)c2ccccc2-c2ccc(-c3cccc(N(c4cccc(-c5ccccc5-c5ccc6ccccc6c5)c4)c4ccc(-c5ccc6ccccc6c5)c5oc6ccccc6c45)c3)cc21 ; [2H]c1c([2H])c([2H])c2c(oc3c(-c4c([2H])c([2H])c([2H])c5c([2H])c([2H])c([2H])c([2H])c45)c([2H])c([2H])c(N(c4ccccc4)c4cccc(-c5ccccc5-c5ccc6ccccc6c5)c4)c32)c1[2H] ; [2H]c1c([2H])c([2H])c(N(c2c([2H])c([2H])c([2H])c(-c3c([2H])c([2H])c([2H])c([2H])c3-c3c([2H])c([2H])c4c([2H])c([2H])c([2H])c([2H])c4c3[2H])c2[2H])c2ccc(-c3cccc4ccccc34)c3oc4ccccc4c23)c([2H])c1[2H]]

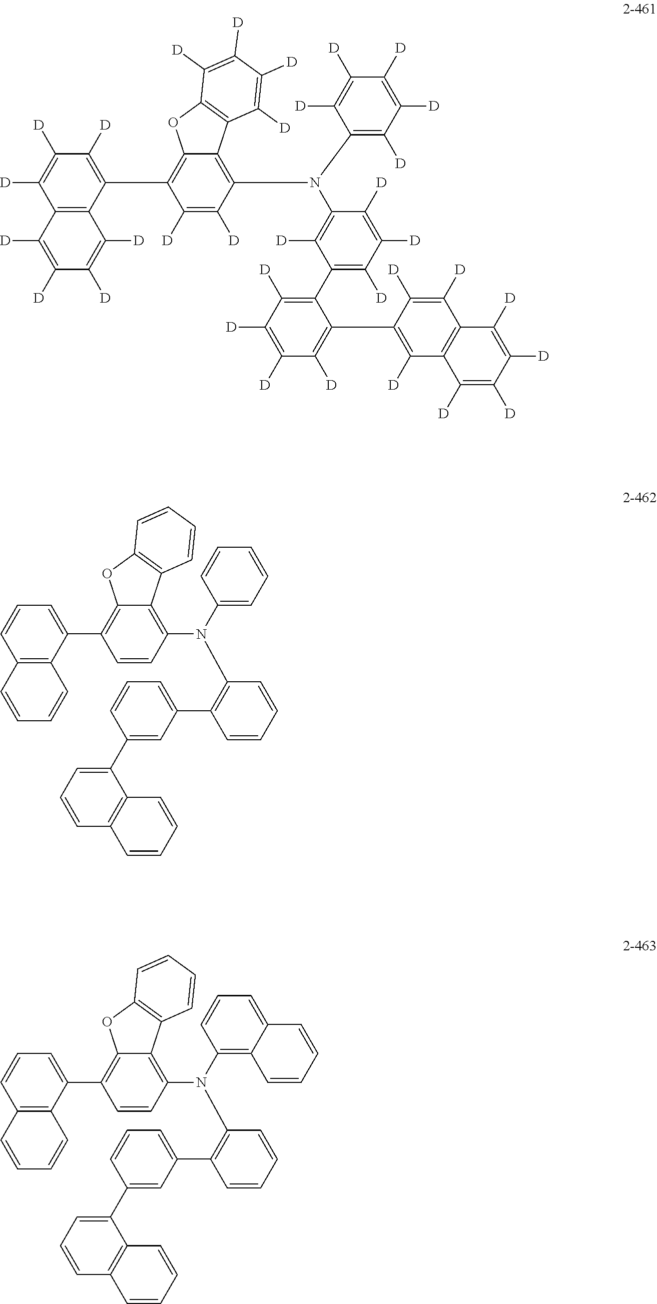 [SMILES: [2H]c1c([2H])c([2H])c(N(c2c([2H])c([2H])c([2H])c(-c3c([2H])c([2H])c([2H])c([2H])c3-c3c([2H])c([2H])c4c([2H])c([2H])c([2H])c([2H])c4c3[2H])c2[2H])c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H] ; c1ccc(N(c2ccccc2-c2cccc(-c3cccc4ccccc34)c2)c2ccc(-c3cccc4ccccc34)c3oc4ccccc4c23)cc1 ; c1cc(-c2ccccc2N(c2cccc3ccccc23)c2ccc(-c3cccc4ccccc34)c3oc4ccccc4c23)cc(-c2cccc3ccccc23)c1]

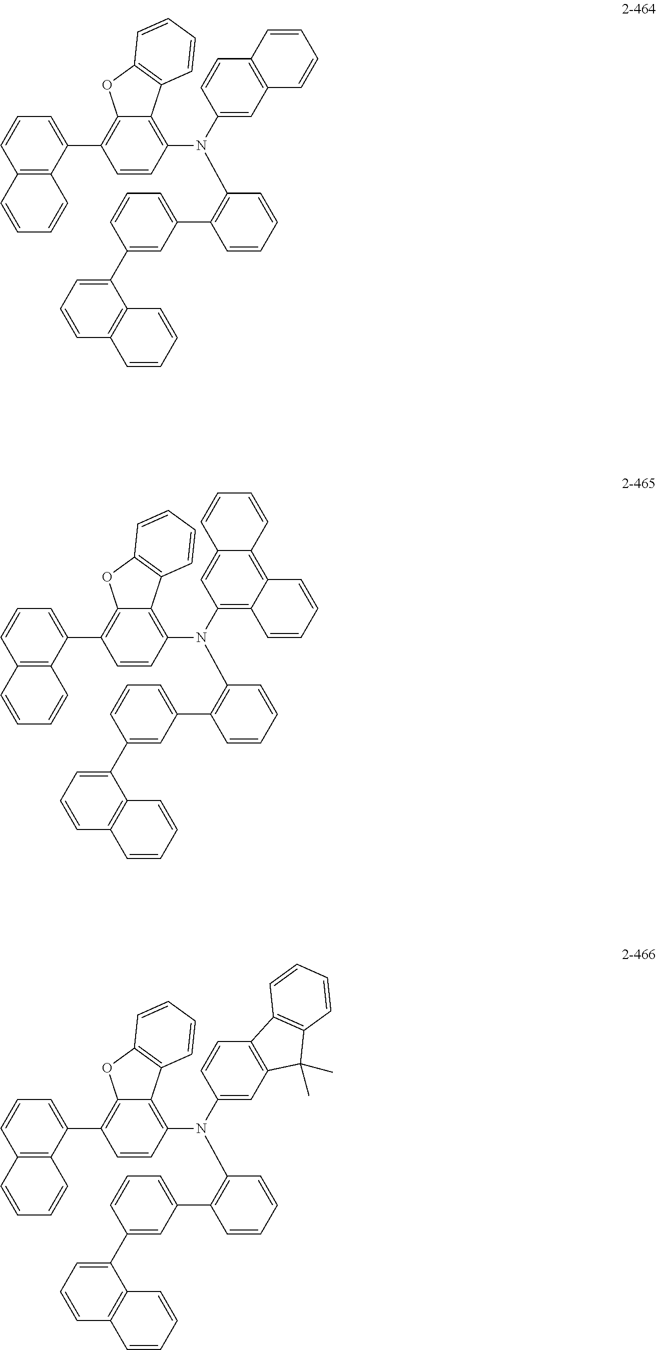 [SMILES: c1cc(-c2ccccc2N(c2ccc3ccccc3c2)c2ccc(-c3cccc4ccccc34)c3oc4ccccc4c23)cc(-c2cccc3ccccc23)c1 ; c1cc(-c2ccccc2N(c2cc3ccccc3c3ccccc23)c2ccc(-c3cccc4ccccc34)c3oc4ccccc4c23)cc(-c2cccc3ccccc23)c1 ; CC1(C)c2ccccc2-c2ccc(N(c3ccccc3-c3cccc(-c4cccc5ccccc45)c3)c3ccc(-c4cccc5ccccc45)c4oc5ccccc5c34)cc21]

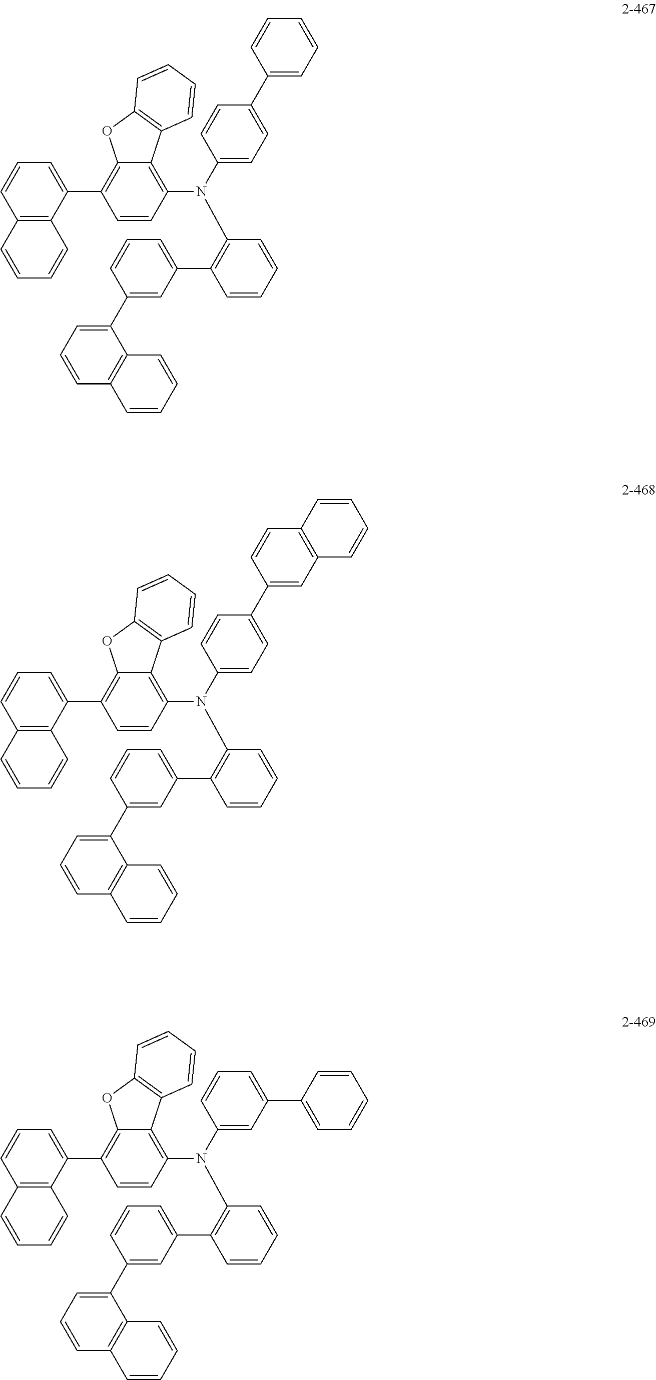 [SMILES: c1ccc(-c2ccc(N(c3ccccc3-c3cccc(-c4cccc5ccccc45)c3)c3ccc(-c4cccc5ccccc45)c4oc5ccccc5c34)cc2)cc1 ; c1cc(-c2ccccc2N(c2ccc(-c3ccc4ccccc4c3)cc2)c2ccc(-c3cccc4ccccc34)c3oc4ccccc4c23)cc(-c2cccc3ccccc23)c1 ; c1ccc(-c2cccc(N(c3ccccc3-c3cccc(-c4cccc5ccccc45)c3)c3ccc(-c4cccc5ccccc45)c4oc5ccccc5c34)c2)cc1]

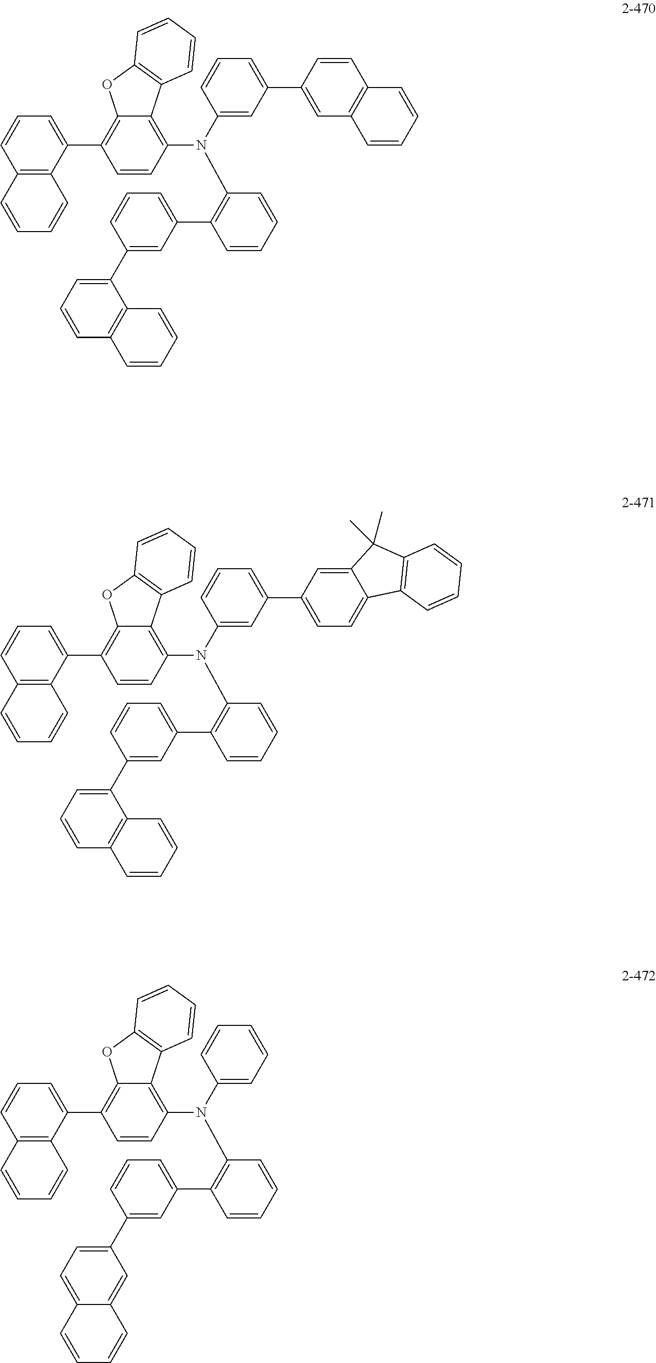 [SMILES: c1cc(-c2ccccc2N(c2cccc(-c3ccc4ccccc4c3)c2)c2ccc(-c3cccc4ccccc34)c3oc4ccccc4c23)cc(-c2cccc3ccccc23)c1 ; CC1(C)c2ccccc2-c2ccc(-c3cccc(N(c4ccccc4-c4cccc(-c5cccc6ccccc56)c4)c4ccc(-c5cccc6ccccc56)c5oc6ccccc6c45)c3)cc21 ; c1ccc(N(c2ccccc2-c2cccc(-c3ccc4ccccc4c3)c2)c2ccc(-c3cccc4ccccc34)c3oc4ccccc4c23)cc1]

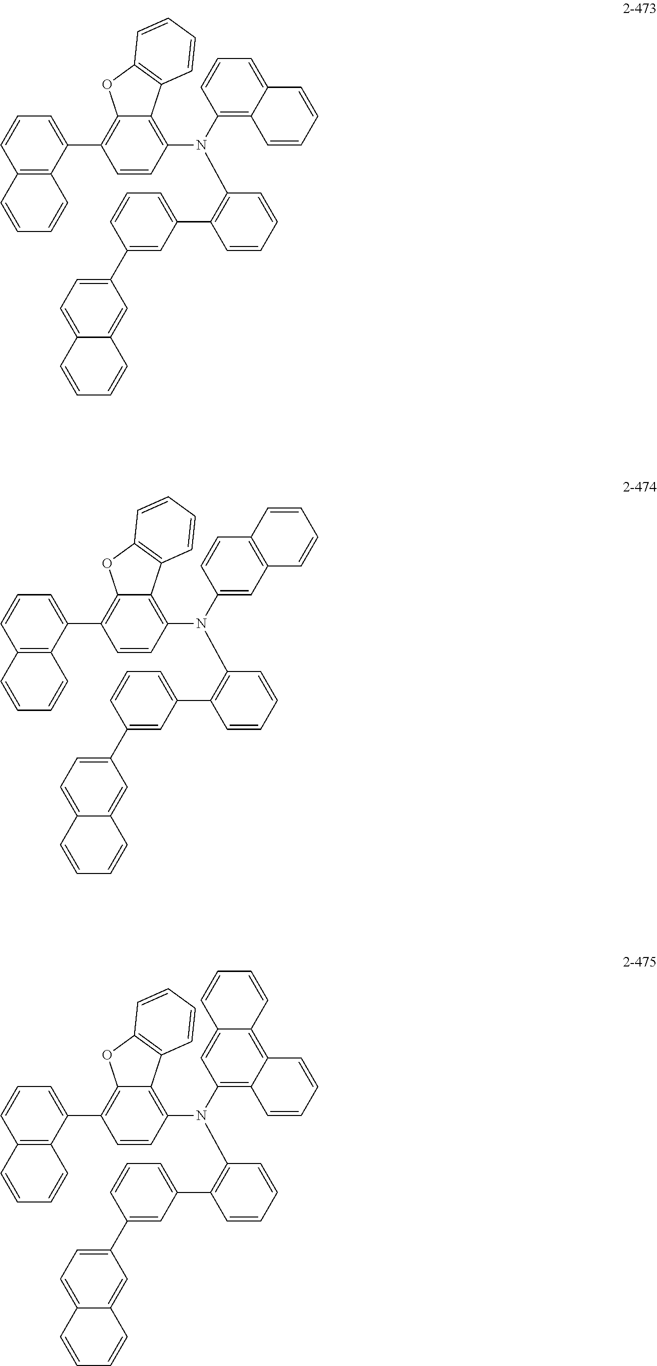 [SMILES: c1cc(-c2ccc3ccccc3c2)cc(-c2ccccc2N(c2cccc3ccccc23)c2ccc(-c3cccc4ccccc34)c3oc4ccccc4c23)c1 ; c1cc(-c2ccc3ccccc3c2)cc(-c2ccccc2N(c2ccc3ccccc3c2)c2ccc(-c3cccc4ccccc34)c3oc4ccccc4c23)c1 ; c1cc(-c2ccc3ccccc3c2)cc(-c2ccccc2N(c2cc3ccccc3c3ccccc23)c2ccc(-c3cccc4ccccc34)c3oc4ccccc4c23)c1]

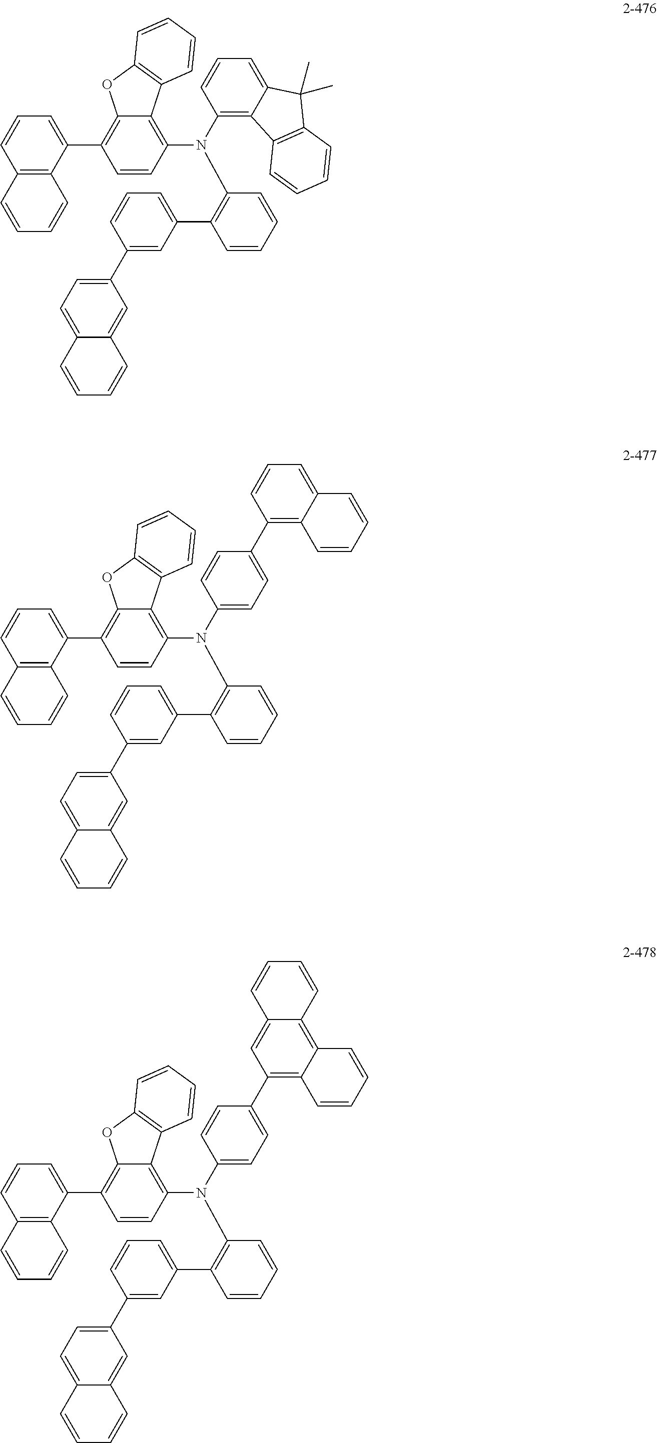 [SMILES: CC1(C)c2ccccc2-c2c(N(c3ccccc3-c3cccc(-c4ccc5ccccc5c4)c3)c3ccc(-c4cccc5ccccc45)c4oc5ccccc5c34)cccc21 ; c1cc(-c2ccc3ccccc3c2)cc(-c2ccccc2N(c2ccc(-c3cccc4ccccc34)cc2)c2ccc(-c3cccc4ccccc34)c3oc4ccccc4c23)c1 ; c1cc(-c2ccc3ccccc3c2)cc(-c2ccccc2N(c2ccc(-c3cc4ccccc4c4ccccc34)cc2)c2ccc(-c3cccc4ccccc34)c3oc4ccccc4c23)c1]

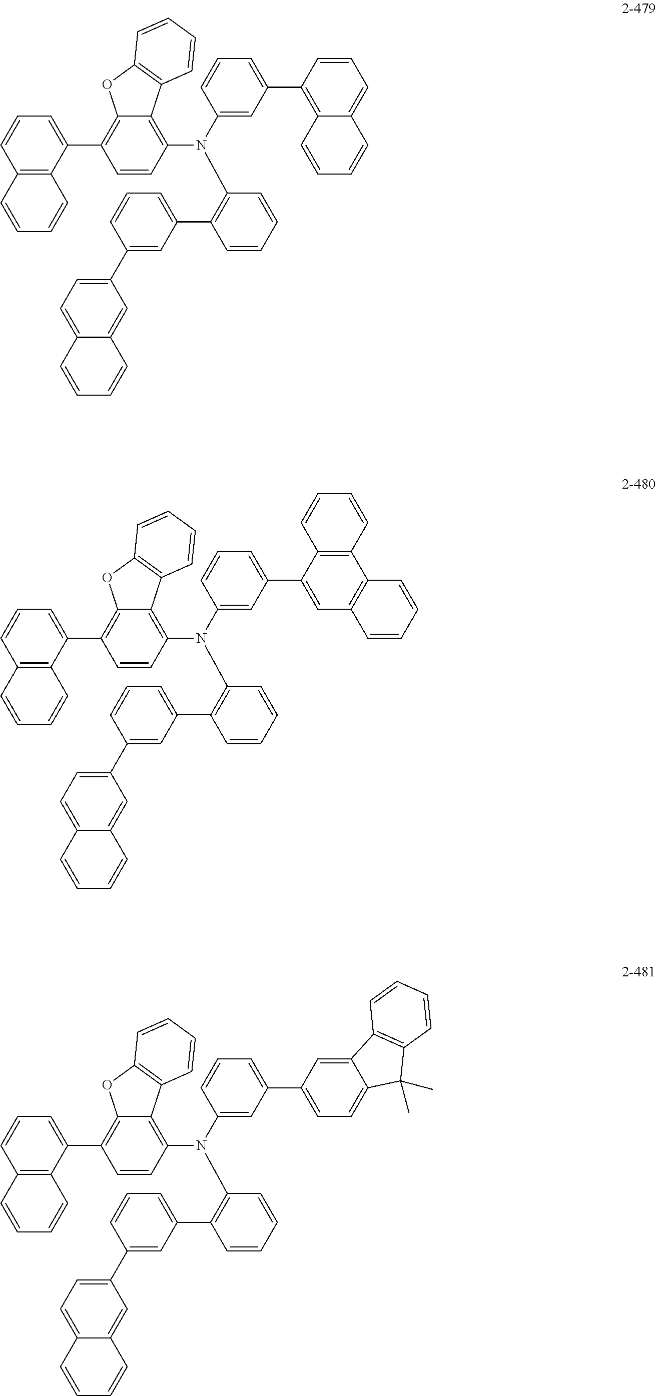 [SMILES: c1cc(-c2ccc3ccccc3c2)cc(-c2ccccc2N(c2cccc(-c3cccc4ccccc34)c2)c2ccc(-c3cccc4ccccc34)c3oc4ccccc4c23)c1 ; c1cc(-c2ccc3ccccc3c2)cc(-c2ccccc2N(c2cccc(-c3cc4ccccc4c4ccccc34)c2)c2ccc(-c3cccc4ccccc34)c3oc4ccccc4c23)c1 ; CC1(C)c2ccccc2-c2cc(-c3cccc(N(c4ccccc4-c4cccc(-c5ccc6ccccc6c5)c4)c4ccc(-c5cccc6ccccc56)c5oc6ccccc6c45)c3)ccc21]

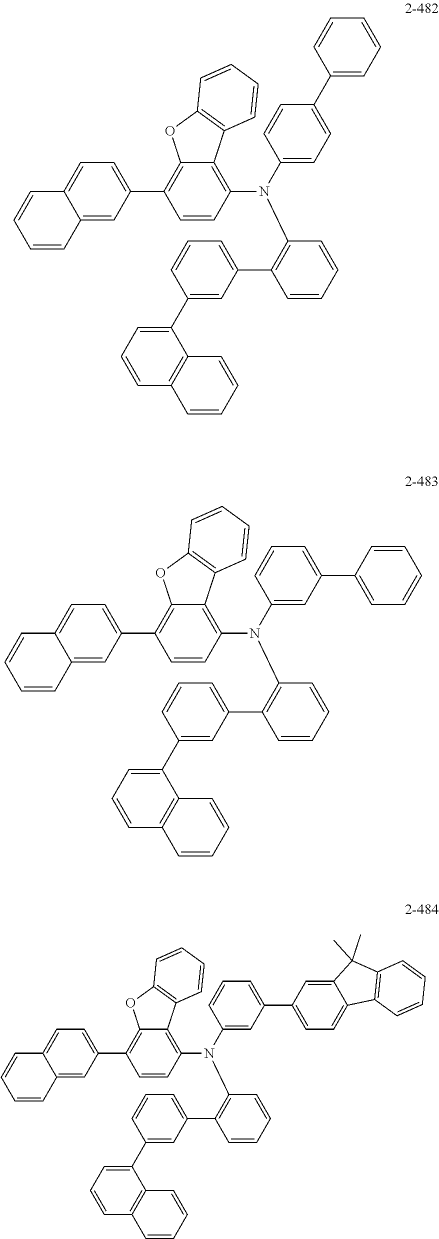 [SMILES: c1ccc(-c2ccc(N(c3ccccc3-c3cccc(-c4cccc5ccccc45)c3)c3ccc(-c4ccc5ccccc5c4)c4oc5ccccc5c34)cc2)cc1 ; c1ccc(-c2cccc(N(c3ccccc3-c3cccc(-c4cccc5ccccc45)c3)c3ccc(-c4ccc5ccccc5c4)c4oc5ccccc5c34)c2)cc1 ; CC1(C)c2ccccc2-c2ccc(-c3cccc(N(c4ccccc4-c4cccc(-c5cccc6ccccc56)c4)c4ccc(-c5ccc6ccccc6c5)c5oc6ccccc6c45)c3)cc21]

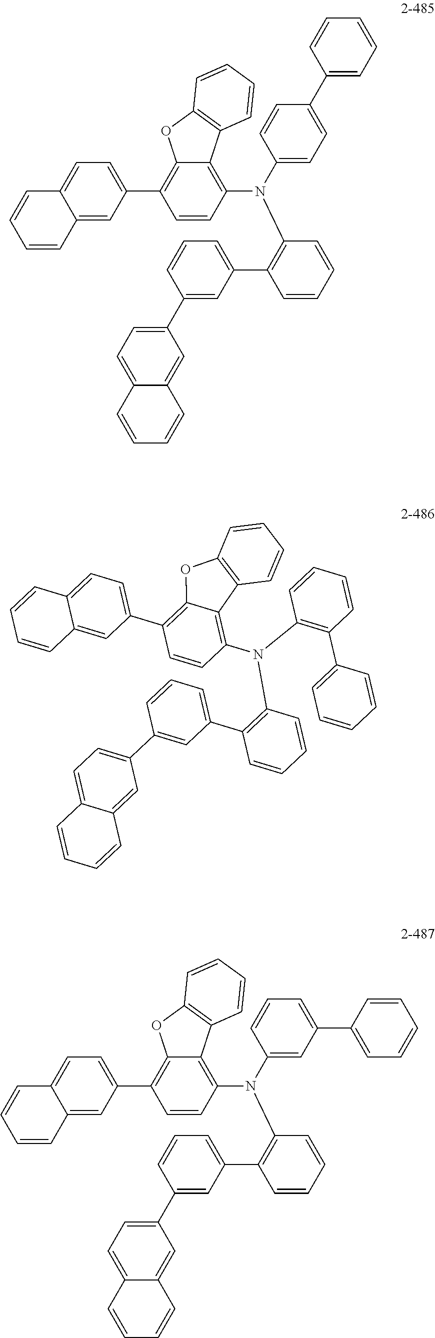 [SMILES: c1ccc(-c2ccc(N(c3ccccc3-c3cccc(-c4ccc5ccccc5c4)c3)c3ccc(-c4ccc5ccccc5c4)c4oc5ccccc5c34)cc2)cc1 ; c1ccc(-c2ccccc2N(c2ccccc2-c2cccc(-c3ccc4ccccc4c3)c2)c2ccc(-c3ccc4ccccc4c3)c3oc4ccccc4c23)cc1 ; c1ccc(-c2cccc(N(c3ccccc3-c3cccc(-c4ccc5ccccc5c4)c3)c3ccc(-c4ccc5ccccc5c4)c4oc5ccccc5c34)c2)cc1]

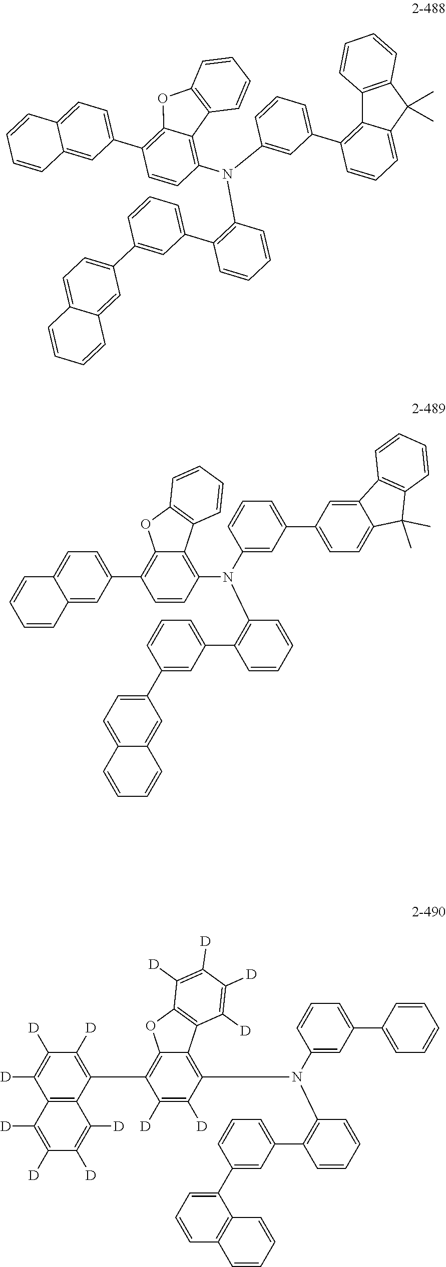 [SMILES: CC1(C)c2ccccc2-c2c(-c3cccc(N(c4ccccc4-c4cccc(-c5ccc6ccccc6c5)c4)c4ccc(-c5ccc6ccccc6c5)c5oc6ccccc6c45)c3)cccc21 ; CC1(C)c2ccccc2-c2cc(-c3cccc(N(c4ccccc4-c4cccc(-c5ccc6ccccc6c5)c4)c4ccc(-c5ccc6ccccc6c5)c5oc6ccccc6c45)c3)ccc21 ; [2H]c1c([2H])c([2H])c2c(oc3c(-c4c([2H])c([2H])c([2H])c5c([2H])c([2H])c([2H])c([2H])c45)c([2H])c([2H])c(N(c4cccc(-c5ccccc5)c4)c4ccccc4-c4cccc(-c5cccc6ccccc56)c4)c32)c1[2H]]

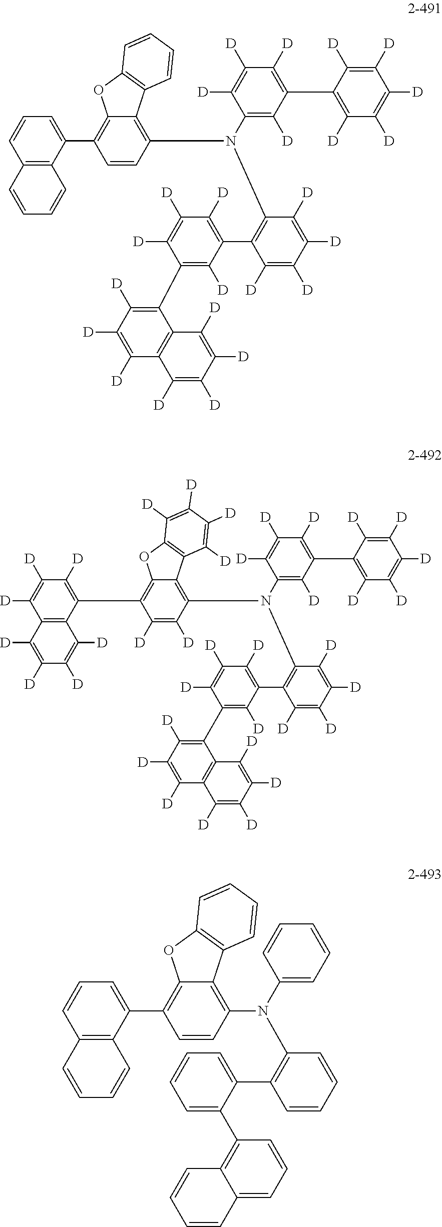

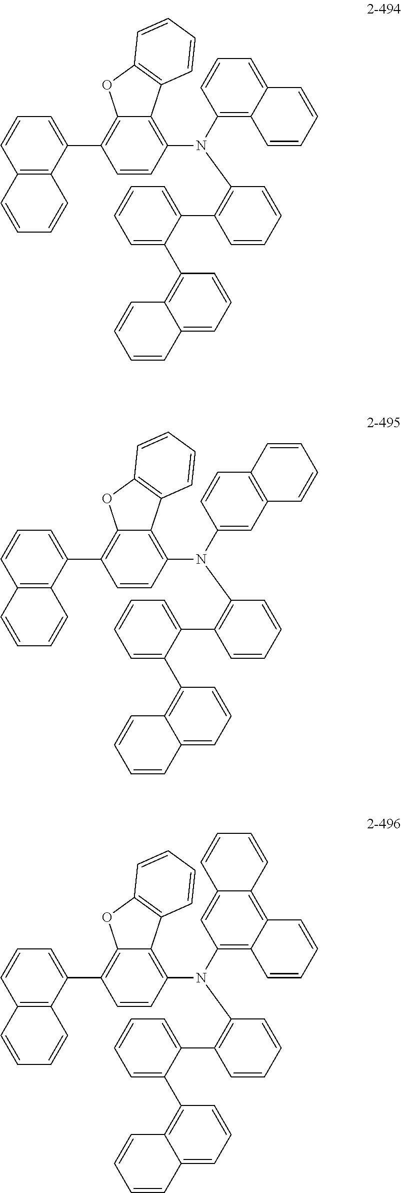 [SMILES: c1ccc(-c2cccc3ccccc23)c(-c2ccccc2N(c2cccc3ccccc23)c2ccc(-c3cccc4ccccc34)c3oc4ccccc4c23)c1 ; c1ccc(-c2cccc3ccccc23)c(-c2ccccc2N(c2ccc3ccccc3c2)c2ccc(-c3cccc4ccccc34)c3oc4ccccc4c23)c1 ; c1ccc(-c2cccc3ccccc23)c(-c2ccccc2N(c2cc3ccccc3c3ccccc23)c2ccc(-c3cccc4ccccc34)c3oc4ccccc4c23)c1]

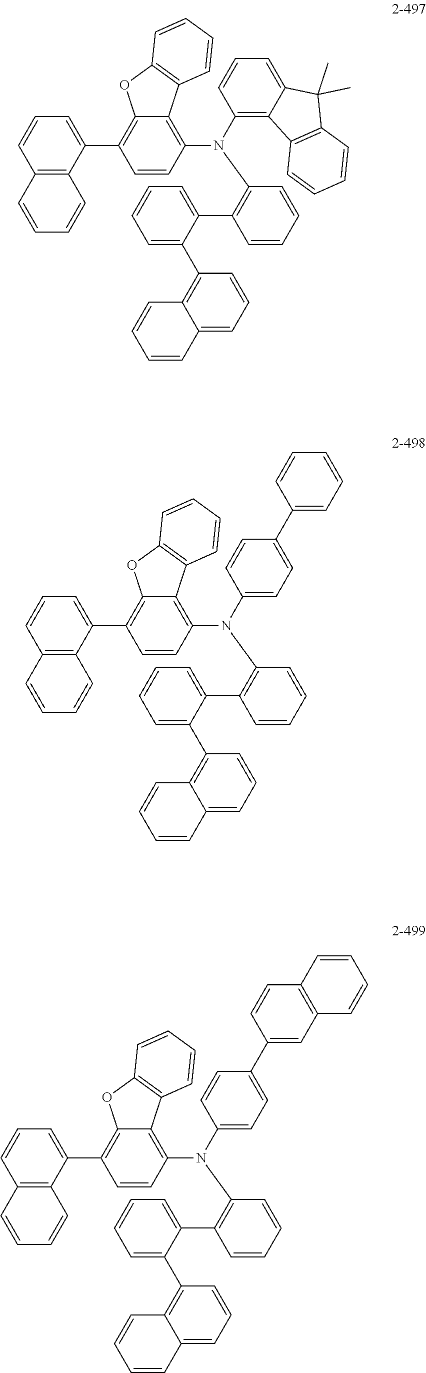 [SMILES: CC1(C)c2ccccc2-c2c(N(c3ccccc3-c3ccccc3-c3cccc4ccccc34)c3ccc(-c4cccc5ccccc45)c4oc5ccccc5c34)cccc21 ; c1ccc(-c2ccc(N(c3ccccc3-c3ccccc3-c3cccc4ccccc34)c3ccc(-c4cccc5ccccc45)c4oc5ccccc5c34)cc2)cc1 ; c1ccc(-c2cccc3ccccc23)c(-c2ccccc2N(c2ccc(-c3ccc4ccccc4c3)cc2)c2ccc(-c3cccc4ccccc34)c3oc4ccccc4c23)c1]

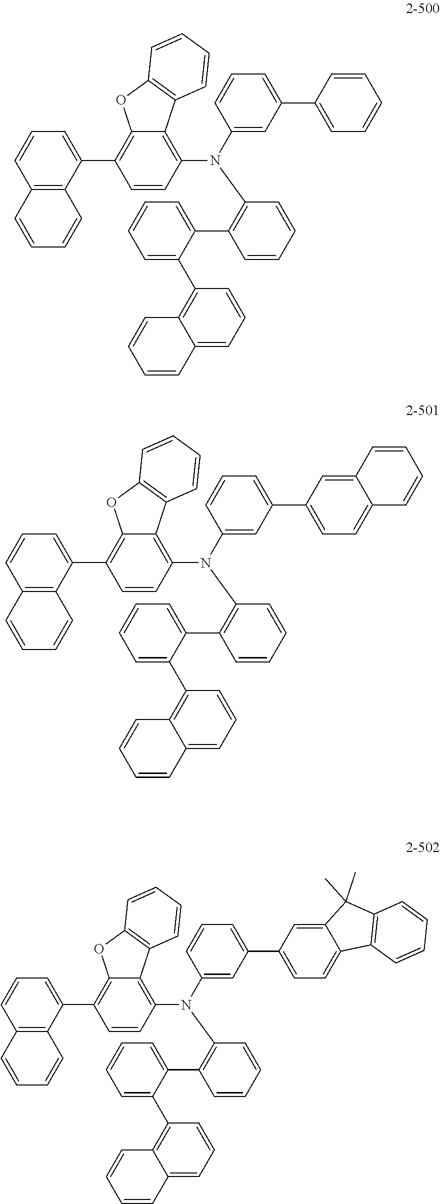 [SMILES: c1ccc(-c2cccc(N(c3ccccc3-c3ccccc3-c3cccc4ccccc34)c3ccc(-c4cccc5ccccc45)c4oc5ccccc5c34)c2)cc1 ; c1cc(-c2ccc3ccccc3c2)cc(N(c2ccccc2-c2ccccc2-c2cccc3ccccc23)c2ccc(-c3cccc4ccccc34)c3oc4ccccc4c23)c1 ; CC1(C)c2ccccc2-c2ccc(-c3cccc(N(c4ccccc4-c4ccccc4-c4cccc5ccccc45)c4ccc(-c5cccc6ccccc56)c5oc6ccccc6c45)c3)cc21]

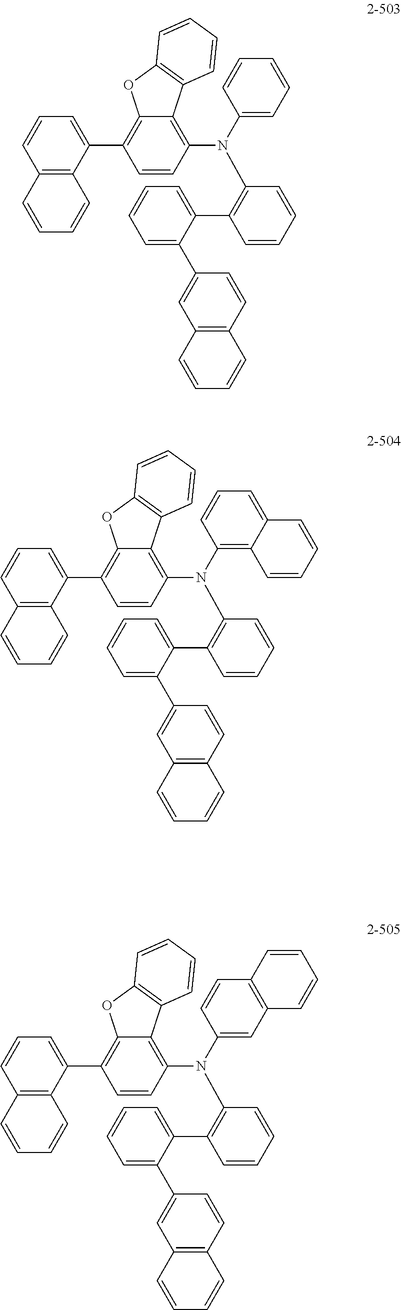 [SMILES: c1ccc(N(c2ccccc2-c2ccccc2-c2ccc3ccccc3c2)c2ccc(-c3cccc4ccccc34)c3oc4ccccc4c23)cc1 ; c1ccc(-c2ccccc2N(c2cccc3ccccc23)c2ccc(-c3cccc4ccccc34)c3oc4ccccc4c23)c(-c2ccc3ccccc3c2)c1 ; c1ccc(-c2ccccc2N(c2ccc3ccccc3c2)c2ccc(-c3cccc4ccccc34)c3oc4ccccc4c23)c(-c2ccc3ccccc3c2)c1]

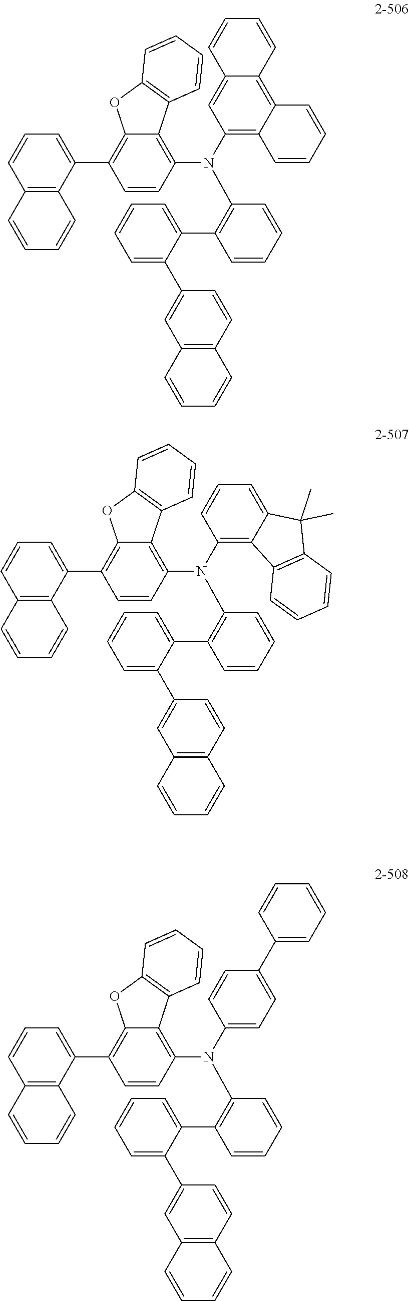

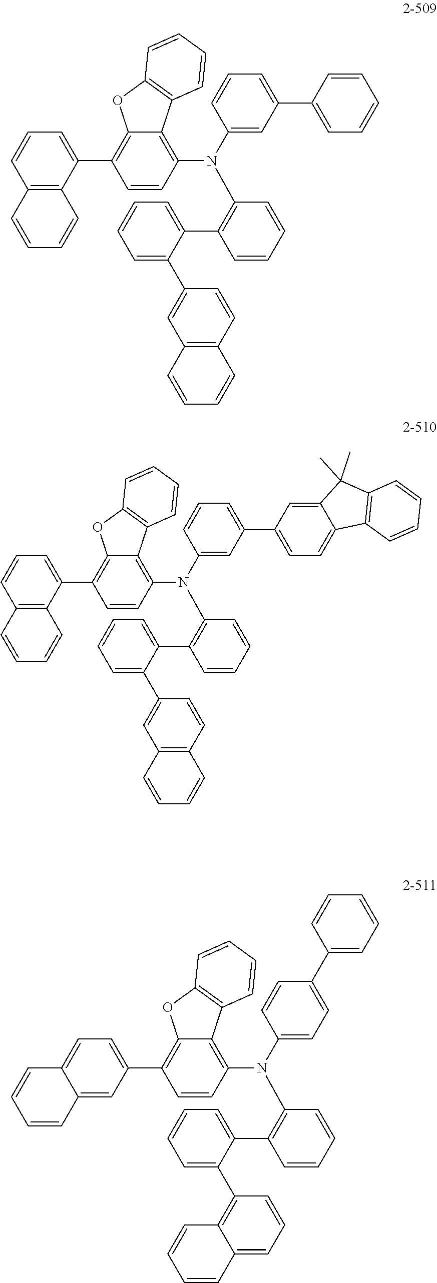 [SMILES: c1ccc(-c2cccc(N(c3ccccc3-c3ccccc3-c3ccc4ccccc4c3)c3ccc(-c4cccc5ccccc45)c4oc5ccccc5c34)c2)cc1 ; CC1(C)c2ccccc2-c2ccc(-c3cccc(N(c4ccccc4-c4ccccc4-c4ccc5ccccc5c4)c4ccc(-c5cccc6ccccc56)c5oc6ccccc6c45)c3)cc21 ; c1ccc(-c2ccc(N(c3ccccc3-c3ccccc3-c3cccc4ccccc34)c3ccc(-c4ccc5ccccc5c4)c4oc5ccccc5c34)cc2)cc1]

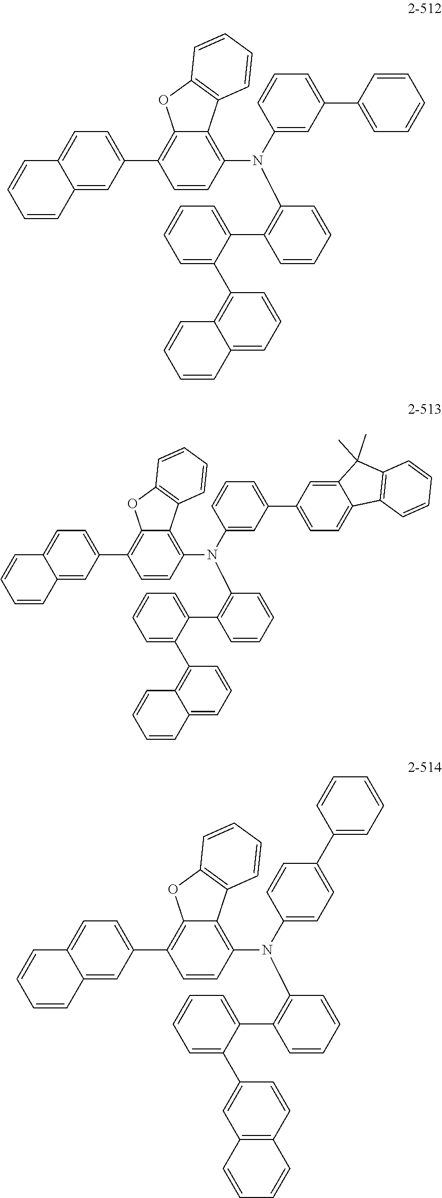 [SMILES: c1ccc(-c2cccc(N(c3ccccc3-c3ccccc3-c3cccc4ccccc34)c3ccc(-c4ccc5ccccc5c4)c4oc5ccccc5c34)c2)cc1 ; CC1(C)c2ccccc2-c2ccc(-c3cccc(N(c4ccccc4-c4ccccc4-c4cccc5ccccc45)c4ccc(-c5ccc6ccccc6c5)c5oc6ccccc6c45)c3)cc21 ; c1ccc(-c2ccc(N(c3ccccc3-c3ccccc3-c3ccc4ccccc4c3)c3ccc(-c4ccc5ccccc5c4)c4oc5ccccc5c34)cc2)cc1]

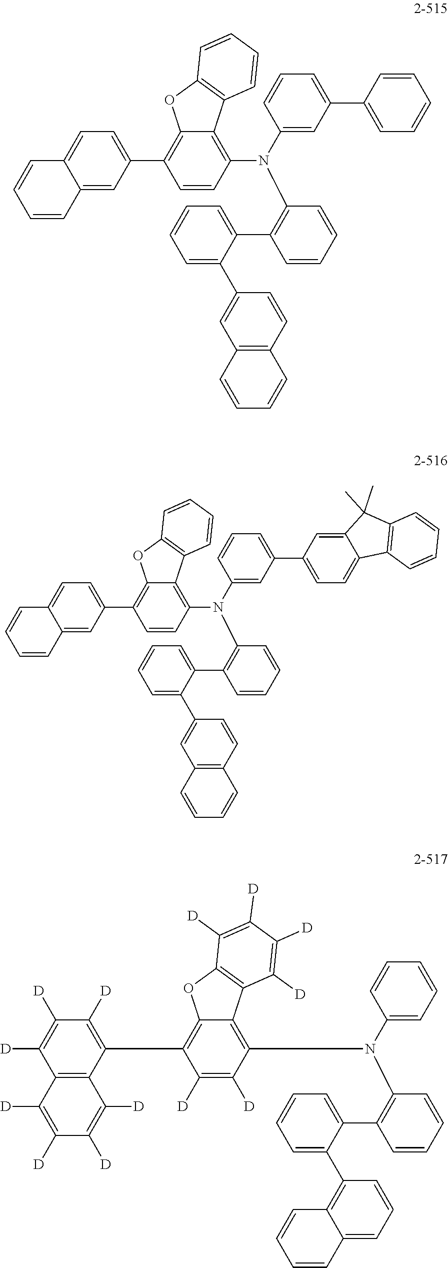 [SMILES: c1ccc(-c2cccc(N(c3ccccc3-c3ccccc3-c3ccc4ccccc4c3)c3ccc(-c4ccc5ccccc5c4)c4oc5ccccc5c34)c2)cc1 ; CC1(C)c2ccccc2-c2ccc(-c3cccc(N(c4ccccc4-c4ccccc4-c4ccc5ccccc5c4)c4ccc(-c5ccc6ccccc6c5)c5oc6ccccc6c45)c3)cc21 ; [2H]c1c([2H])c([2H])c2c(oc3c(-c4c([2H])c([2H])c([2H])c5c([2H])c([2H])c([2H])c([2H])c45)c([2H])c([2H])c(N(c4ccccc4)c4ccccc4-c4ccccc4-c4cccc5ccccc45)c32)c1[2H]]

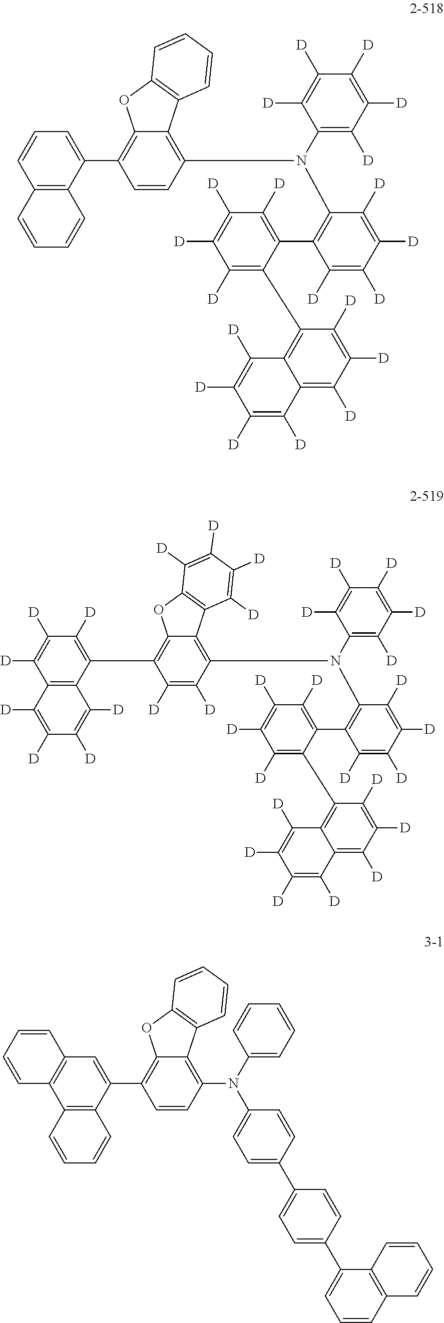

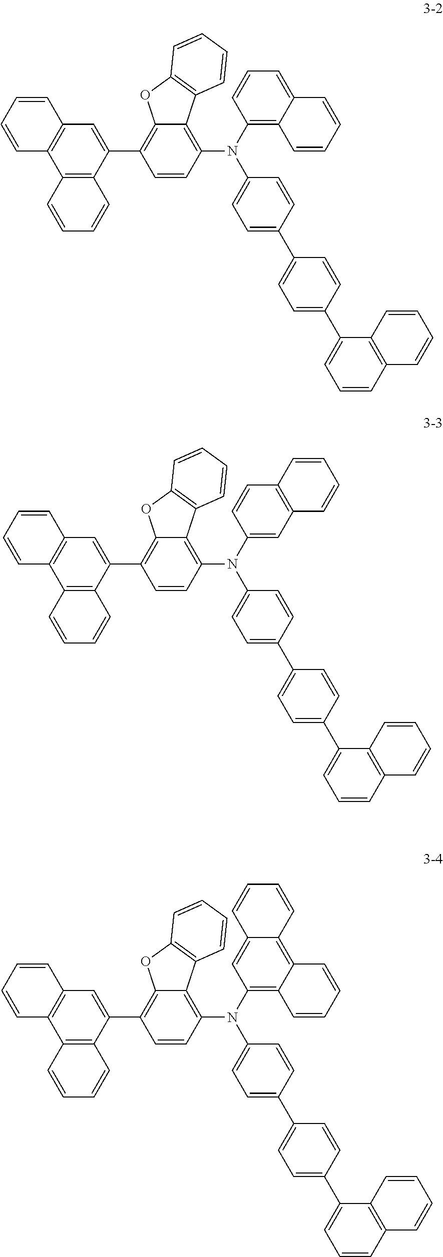 [SMILES: c1ccc2c(-c3ccc(-c4ccc(N(c5cccc6ccccc56)c5ccc(-c6cc7ccccc7c7ccccc67)c6oc7ccccc7c56)cc4)cc3)cccc2c1 ; c1ccc2cc(N(c3ccc(-c4ccc(-c5cccc6ccccc56)cc4)cc3)c3ccc(-c4cc5ccccc5c5ccccc45)c4oc5ccccc5c34)ccc2c1 ; c1ccc2c(-c3ccc(-c4ccc(N(c5cc6ccccc6c6ccccc56)c5ccc(-c6cc7ccccc7c7ccccc67)c6oc7ccccc7c56)cc4)cc3)cccc2c1]

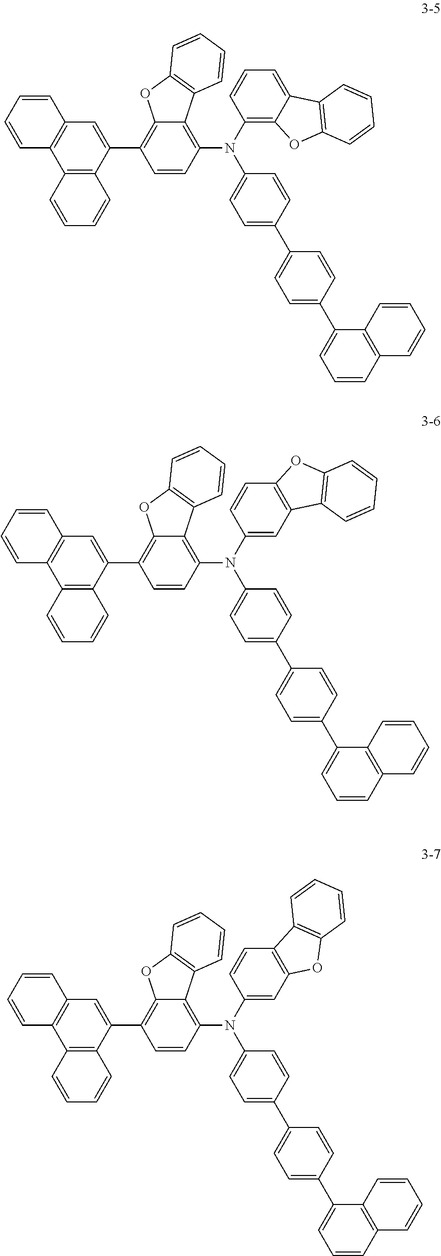 [SMILES: c1ccc2c(-c3ccc(-c4ccc(N(c5cccc6c5oc5ccccc56)c5ccc(-c6cc7ccccc7c7ccccc67)c6oc7ccccc7c56)cc4)cc3)cccc2c1 ; c1ccc2c(-c3ccc(-c4ccc(N(c5ccc6oc7ccccc7c6c5)c5ccc(-c6cc7ccccc7c7ccccc67)c6oc7ccccc7c56)cc4)cc3)cccc2c1 ; c1ccc2c(-c3ccc(-c4ccc(N(c5ccc6c(c5)oc5ccccc56)c5ccc(-c6cc7ccccc7c7ccccc67)c6oc7ccccc7c56)cc4)cc3)cccc2c1]

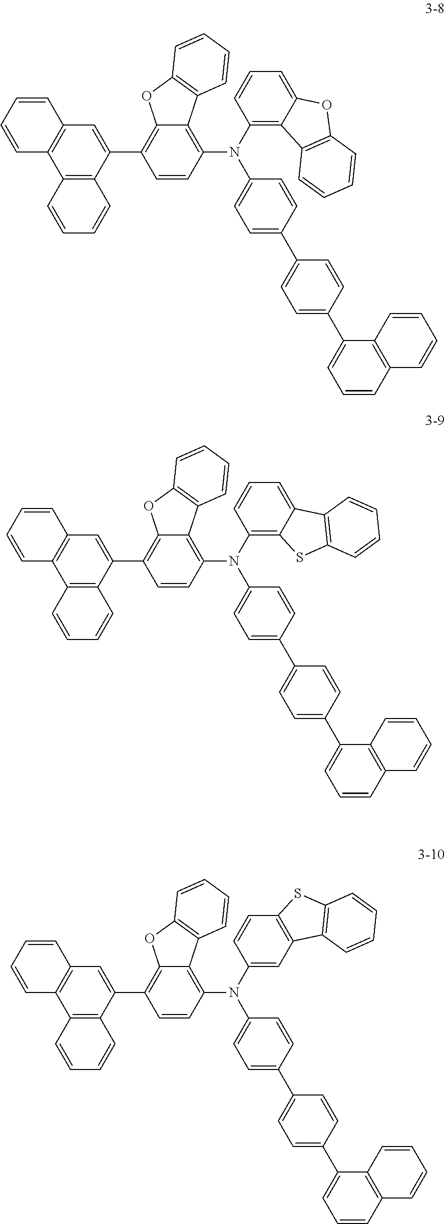 [SMILES: c1ccc2c(-c3ccc(-c4ccc(N(c5cccc6oc7ccccc7c56)c5ccc(-c6cc7ccccc7c7ccccc67)c6oc7ccccc7c56)cc4)cc3)cccc2c1 ; c1ccc2c(-c3ccc(-c4ccc(N(c5cccc6c5sc5ccccc56)c5ccc(-c6cc7ccccc7c7ccccc67)c6oc7ccccc7c56)cc4)cc3)cccc2c1 ; c1ccc2c(-c3ccc(-c4ccc(N(c5ccc6sc7ccccc7c6c5)c5ccc(-c6cc7ccccc7c7ccccc67)c6oc7ccccc7c56)cc4)cc3)cccc2c1]

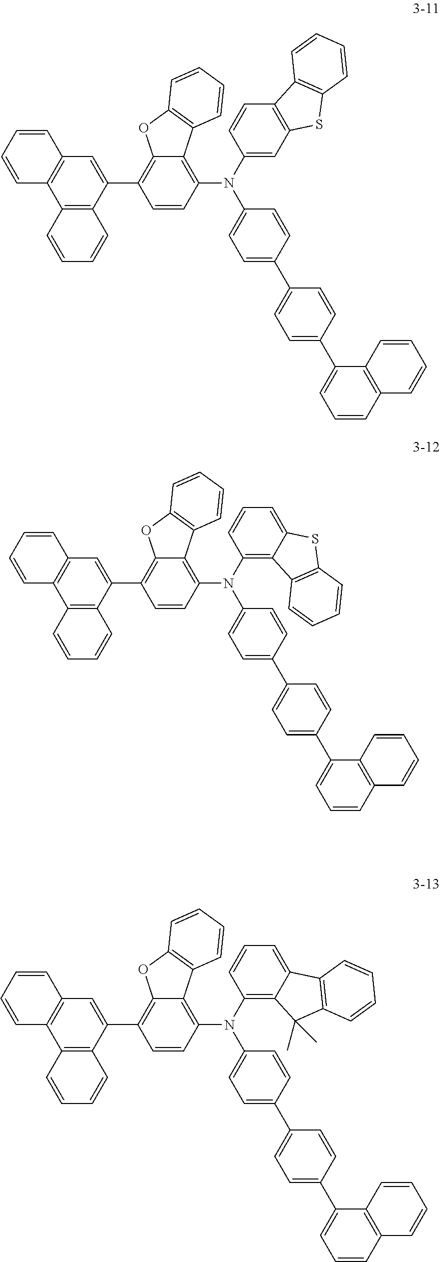 [SMILES: c1ccc2c(-c3ccc(-c4ccc(N(c5ccc6c(c5)sc5ccccc56)c5ccc(-c6cc7ccccc7c7ccccc67)c6oc7ccccc7c56)cc4)cc3)cccc2c1 ; c1ccc2c(-c3ccc(-c4ccc(N(c5cccc6sc7ccccc7c56)c5ccc(-c6cc7ccccc7c7ccccc67)c6oc7ccccc7c56)cc4)cc3)cccc2c1 ; CC1(C)c2ccccc2-c2cccc(N(c3ccc(-c4ccc(-c5cccc6ccccc56)cc4)cc3)c3ccc(-c4cc5ccccc5c5ccccc45)c4oc5ccccc5c34)c21]

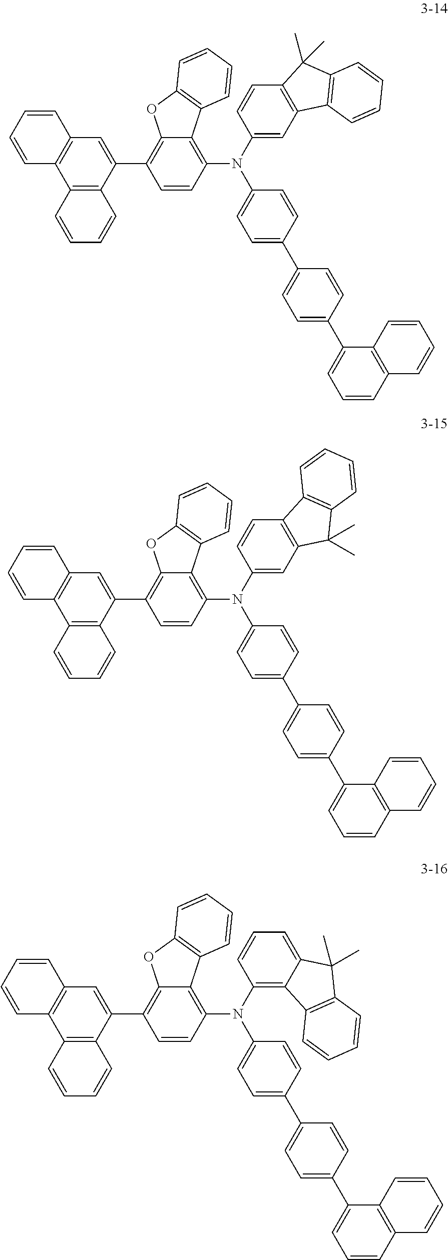 [SMILES: CC1(C)c2ccccc2-c2cc(N(c3ccc(-c4ccc(-c5cccc6ccccc56)cc4)cc3)c3ccc(-c4cc5ccccc5c5ccccc45)c4oc5ccccc5c34)ccc21 ; CC1(C)c2ccccc2-c2ccc(N(c3ccc(-c4ccc(-c5cccc6ccccc56)cc4)cc3)c3ccc(-c4cc5ccccc5c5ccccc45)c4oc5ccccc5c34)cc21 ; CC1(C)c2ccccc2-c2c(N(c3ccc(-c4ccc(-c5cccc6ccccc56)cc4)cc3)c3ccc(-c4cc5ccccc5c5ccccc45)c4oc5ccccc5c34)cccc21]

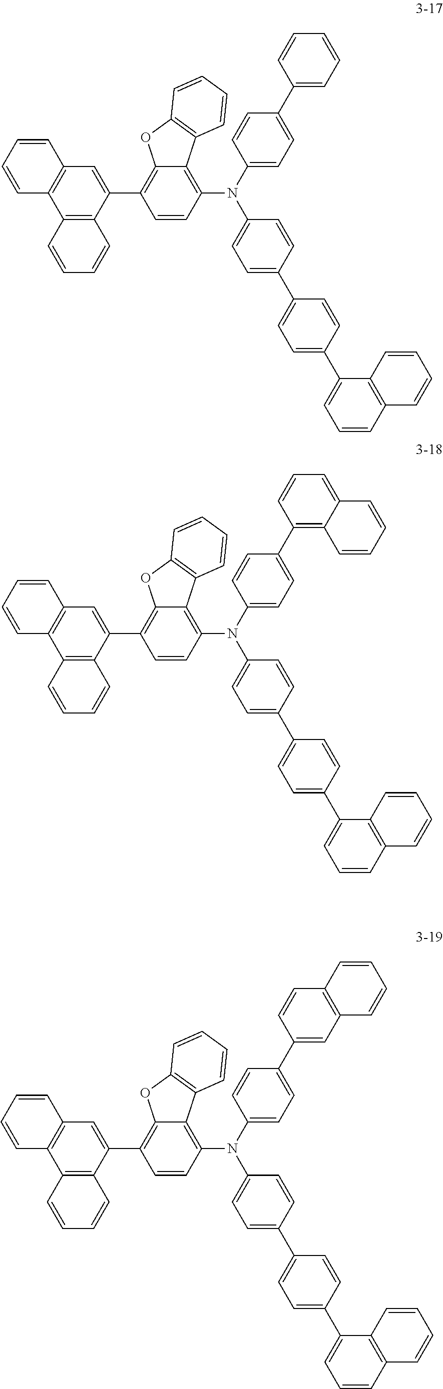 [SMILES: c1ccc(-c2ccc(N(c3ccc(-c4ccc(-c5cccc6ccccc56)cc4)cc3)c3ccc(-c4cc5ccccc5c5ccccc45)c4oc5ccccc5c34)cc2)cc1 ; c1ccc2c(-c3ccc(-c4ccc(N(c5ccc(-c6cccc7ccccc67)cc5)c5ccc(-c6cc7ccccc7c7ccccc67)c6oc7ccccc7c56)cc4)cc3)cccc2c1 ; c1ccc2cc(-c3ccc(N(c4ccc(-c5ccc(-c6cccc7ccccc67)cc5)cc4)c4ccc(-c5cc6ccccc6c6ccccc56)c5oc6ccccc6c45)cc3)ccc2c1]

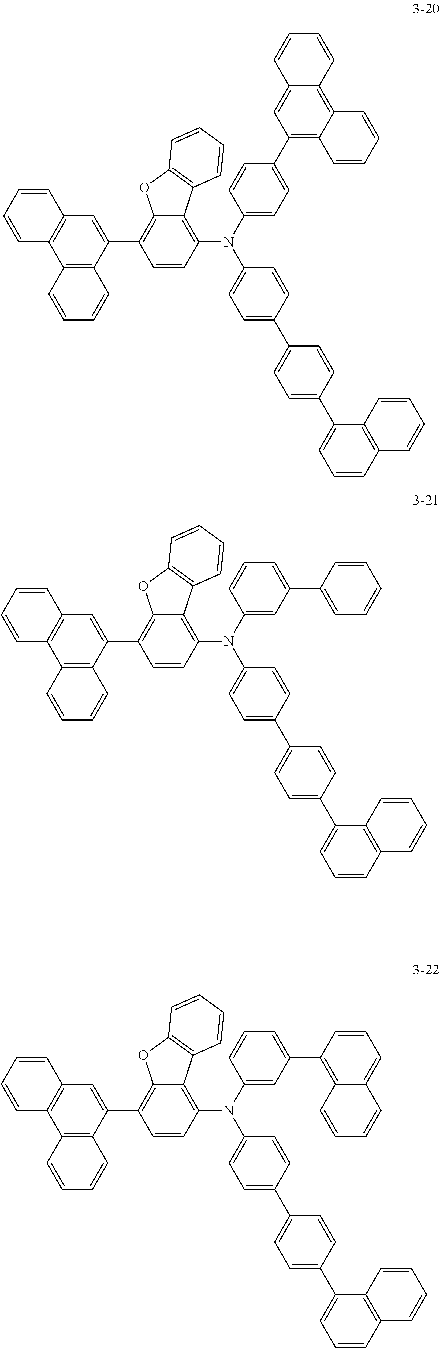 [SMILES: c1ccc2c(-c3ccc(-c4ccc(N(c5ccc(-c6cc7ccccc7c7ccccc67)cc5)c5ccc(-c6cc7ccccc7c7ccccc67)c6oc7ccccc7c56)cc4)cc3)cccc2c1 ; c1ccc(-c2cccc(N(c3ccc(-c4ccc(-c5cccc6ccccc56)cc4)cc3)c3ccc(-c4cc5ccccc5c5ccccc45)c4oc5ccccc5c34)c2)cc1 ; c1cc(-c2cccc3ccccc23)cc(N(c2ccc(-c3ccc(-c4cccc5ccccc45)cc3)cc2)c2ccc(-c3cc4ccccc4c4ccccc34)c3oc4ccccc4c23)c1]

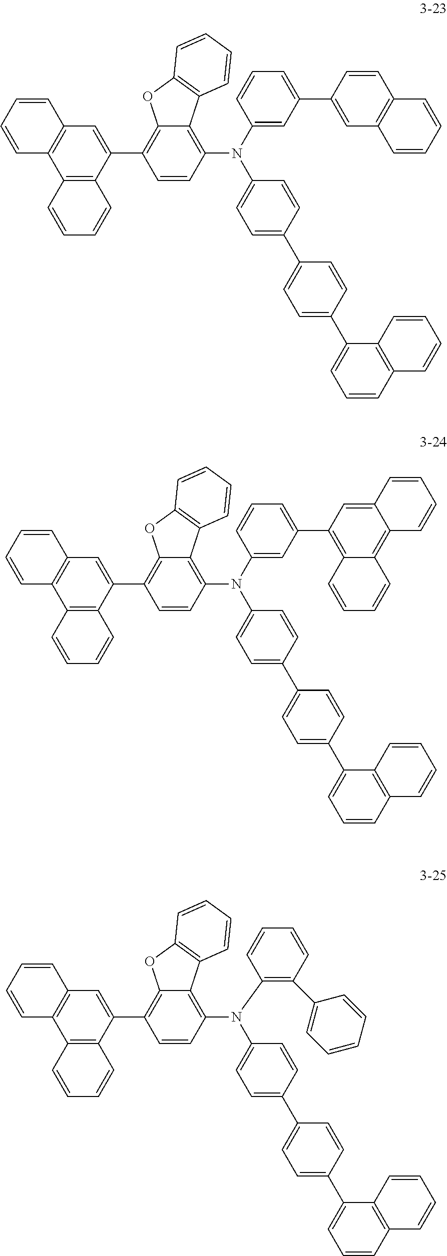 [SMILES: c1cc(-c2ccc3ccccc3c2)cc(N(c2ccc(-c3ccc(-c4cccc5ccccc45)cc3)cc2)c2ccc(-c3cc4ccccc4c4ccccc34)c3oc4ccccc4c23)c1 ; c1cc(-c2cc3ccccc3c3ccccc23)cc(N(c2ccc(-c3ccc(-c4cccc5ccccc45)cc3)cc2)c2ccc(-c3cc4ccccc4c4ccccc34)c3oc4ccccc4c23)c1 ; c1ccc(-c2ccccc2N(c2ccc(-c3ccc(-c4cccc5ccccc45)cc3)cc2)c2ccc(-c3cc4ccccc4c4ccccc34)c3oc4ccccc4c23)cc1]

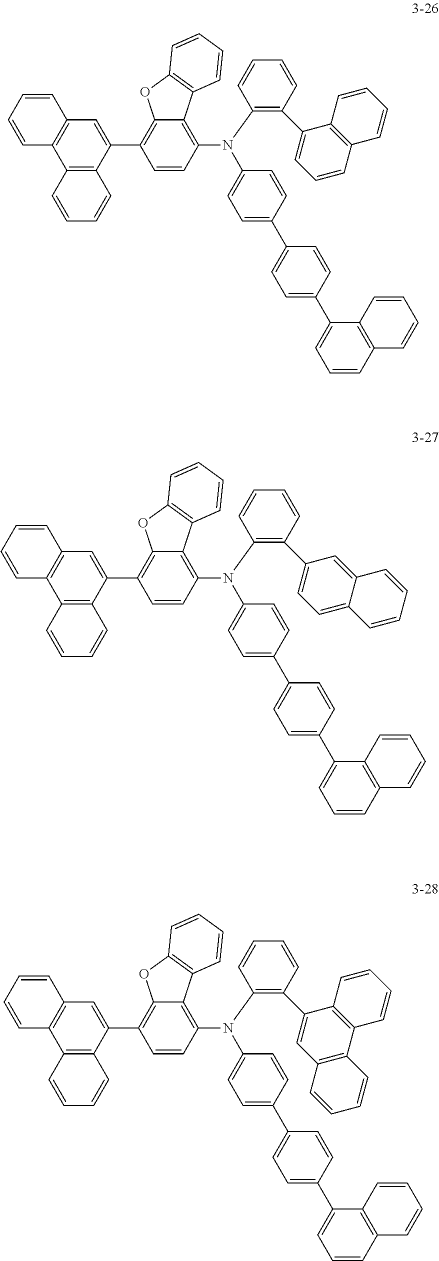 [SMILES: c1ccc(N(c2ccc(-c3ccc(-c4cccc5ccccc45)cc3)cc2)c2ccc(-c3cc4ccccc4c4ccccc34)c3oc4ccccc4c23)c(-c2cccc3ccccc23)c1 ; c1ccc(N(c2ccc(-c3ccc(-c4cccc5ccccc45)cc3)cc2)c2ccc(-c3cc4ccccc4c4ccccc34)c3oc4ccccc4c23)c(-c2ccc3ccccc3c2)c1 ; c1ccc(N(c2ccc(-c3ccc(-c4cccc5ccccc45)cc3)cc2)c2ccc(-c3cc4ccccc4c4ccccc34)c3oc4ccccc4c23)c(-c2cc3ccccc3c3ccccc23)c1]

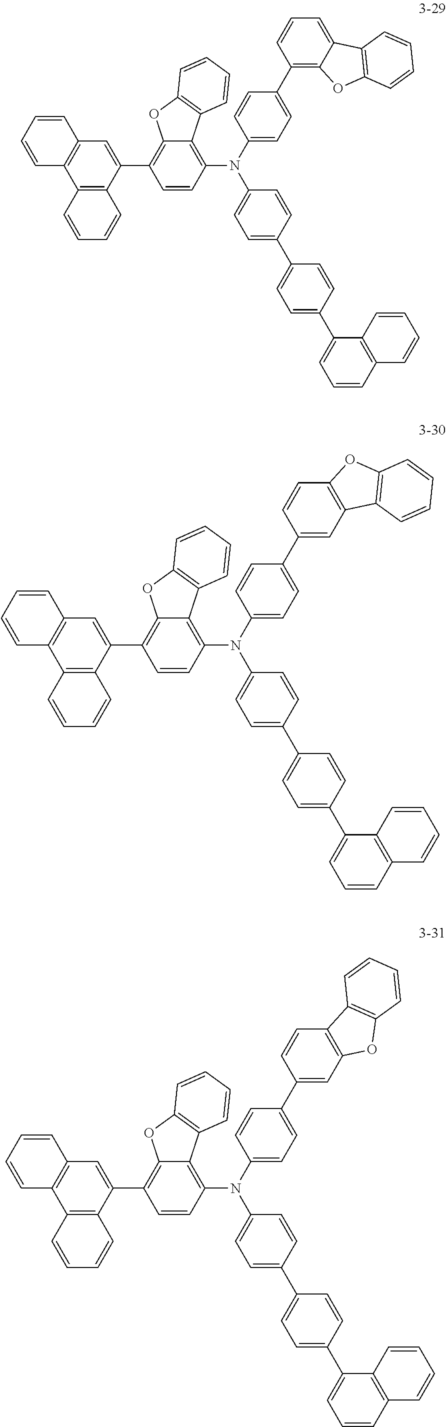 [SMILES: c1ccc2c(-c3ccc(-c4ccc(N(c5ccc(-c6cccc7c6oc6ccccc67)cc5)c5ccc(-c6cc7ccccc7c7ccccc67)c6oc7ccccc7c56)cc4)cc3)cccc2c1 ; c1ccc2c(-c3ccc(-c4ccc(N(c5ccc(-c6ccc7oc8ccccc8c7c6)cc5)c5ccc(-c6cc7ccccc7c7ccccc67)c6oc7ccccc7c56)cc4)cc3)cccc2c1 ; c1ccc2c(-c3ccc(-c4ccc(N(c5ccc(-c6ccc7c(c6)oc6ccccc67)cc5)c5ccc(-c6cc7ccccc7c7ccccc67)c6oc7ccccc7c56)cc4)cc3)cccc2c1]

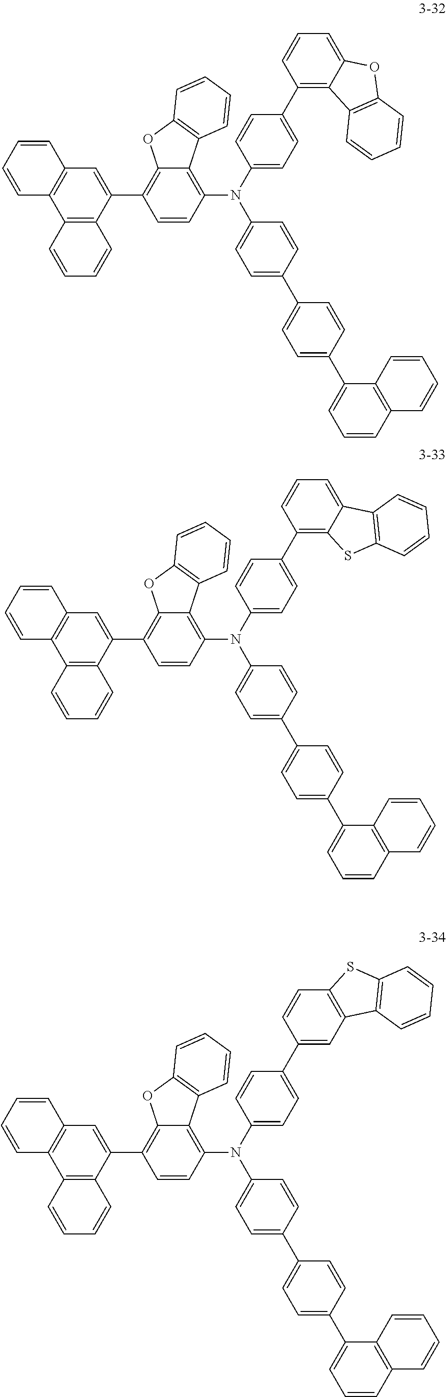 [SMILES: c1ccc2c(-c3ccc(-c4ccc(N(c5ccc(-c6cccc7oc8ccccc8c67)cc5)c5ccc(-c6cc7ccccc7c7ccccc67)c6oc7ccccc7c56)cc4)cc3)cccc2c1 ; c1ccc2c(-c3ccc(-c4ccc(N(c5ccc(-c6cccc7c6sc6ccccc67)cc5)c5ccc(-c6cc7ccccc7c7ccccc67)c6oc7ccccc7c56)cc4)cc3)cccc2c1 ; c1ccc2c(-c3ccc(-c4ccc(N(c5ccc(-c6ccc7sc8ccccc8c7c6)cc5)c5ccc(-c6cc7ccccc7c7ccccc67)c6oc7ccccc7c56)cc4)cc3)cccc2c1]

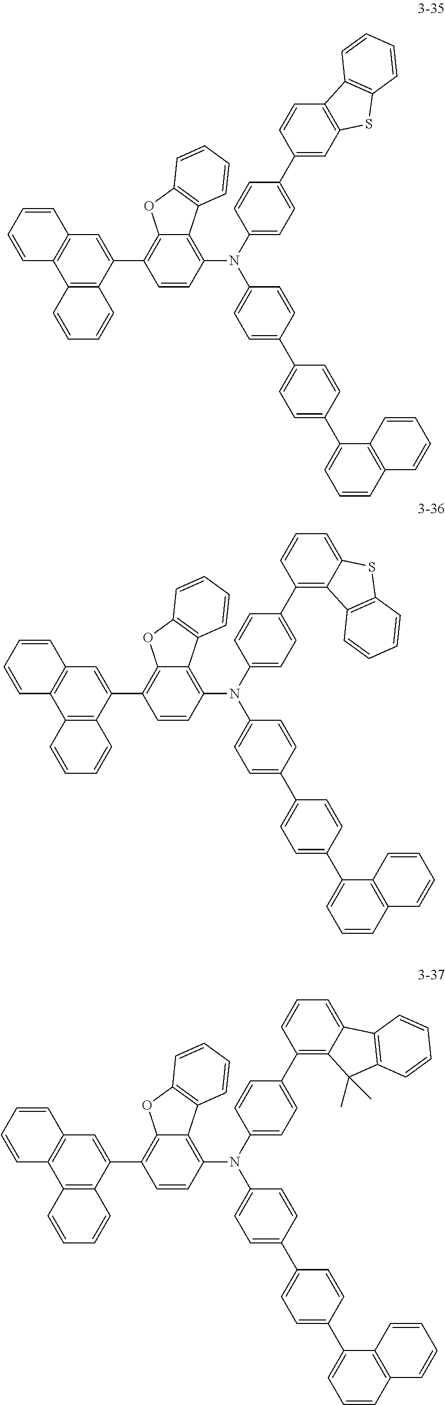

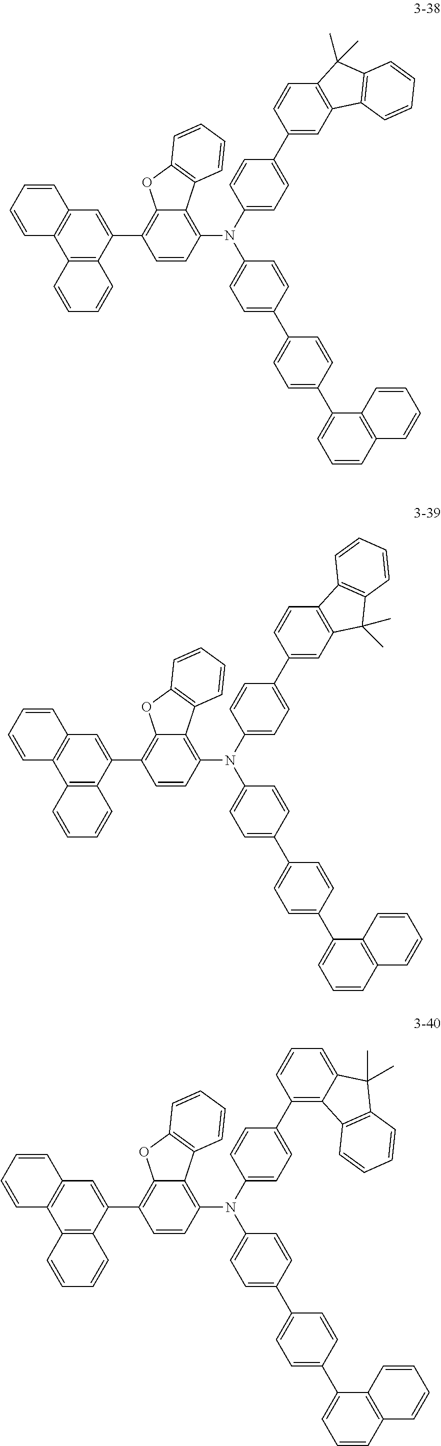 [SMILES: CC1(C)c2ccccc2-c2cc(-c3ccc(N(c4ccc(-c5ccc(-c6cccc7ccccc67)cc5)cc4)c4ccc(-c5cc6ccccc6c6ccccc56)c5oc6ccccc6c45)cc3)ccc21 ; CC1(C)c2ccccc2-c2ccc(-c3ccc(N(c4ccc(-c5ccc(-c6cccc7ccccc67)cc5)cc4)c4ccc(-c5cc6ccccc6c6ccccc56)c5oc6ccccc6c45)cc3)cc21 ; CC1(C)c2ccccc2-c2c(-c3ccc(N(c4ccc(-c5ccc(-c6cccc7ccccc67)cc5)cc4)c4ccc(-c5cc6ccccc6c6ccccc56)c5oc6ccccc6c45)cc3)cccc21]

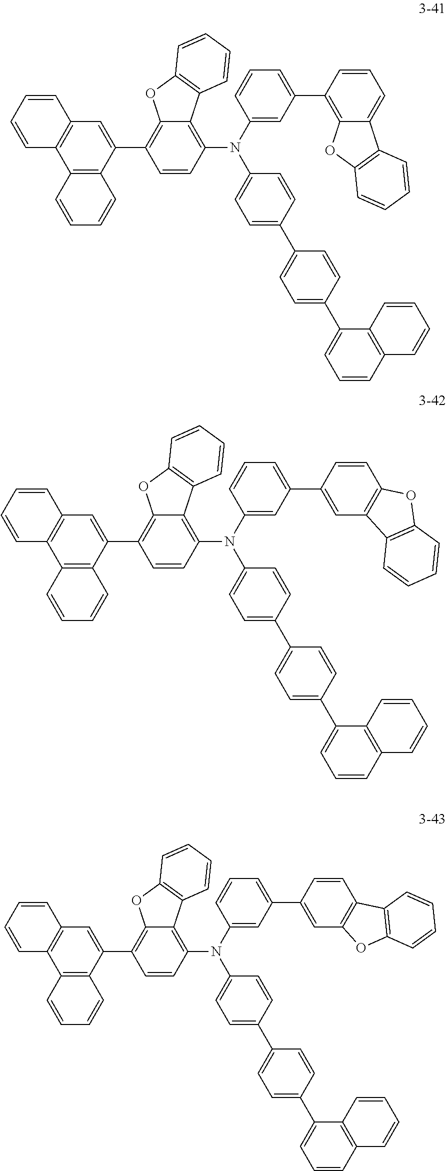 [SMILES: c1cc(-c2cccc3c2oc2ccccc23)cc(N(c2ccc(-c3ccc(-c4cccc5ccccc45)cc3)cc2)c2ccc(-c3cc4ccccc4c4ccccc34)c3oc4ccccc4c23)c1 ; c1cc(-c2ccc3oc4ccccc4c3c2)cc(N(c2ccc(-c3ccc(-c4cccc5ccccc45)cc3)cc2)c2ccc(-c3cc4ccccc4c4ccccc34)c3oc4ccccc4c23)c1 ; c1cc(-c2ccc3c(c2)oc2ccccc23)cc(N(c2ccc(-c3ccc(-c4cccc5ccccc45)cc3)cc2)c2ccc(-c3cc4ccccc4c4ccccc34)c3oc4ccccc4c23)c1]

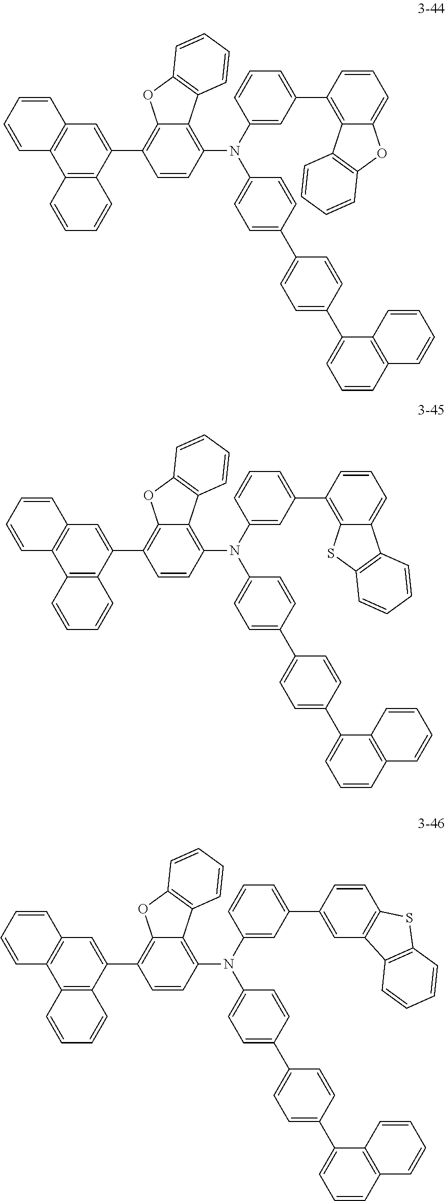 [SMILES: c1cc(-c2cccc3oc4ccccc4c23)cc(N(c2ccc(-c3ccc(-c4cccc5ccccc45)cc3)cc2)c2ccc(-c3cc4ccccc4c4ccccc34)c3oc4ccccc4c23)c1 ; c1cc(-c2cccc3c2sc2ccccc23)cc(N(c2ccc(-c3ccc(-c4cccc5ccccc45)cc3)cc2)c2ccc(-c3cc4ccccc4c4ccccc34)c3oc4ccccc4c23)c1 ; c1cc(-c2ccc3sc4ccccc4c3c2)cc(N(c2ccc(-c3ccc(-c4cccc5ccccc45)cc3)cc2)c2ccc(-c3cc4ccccc4c4ccccc34)c3oc4ccccc4c23)c1]

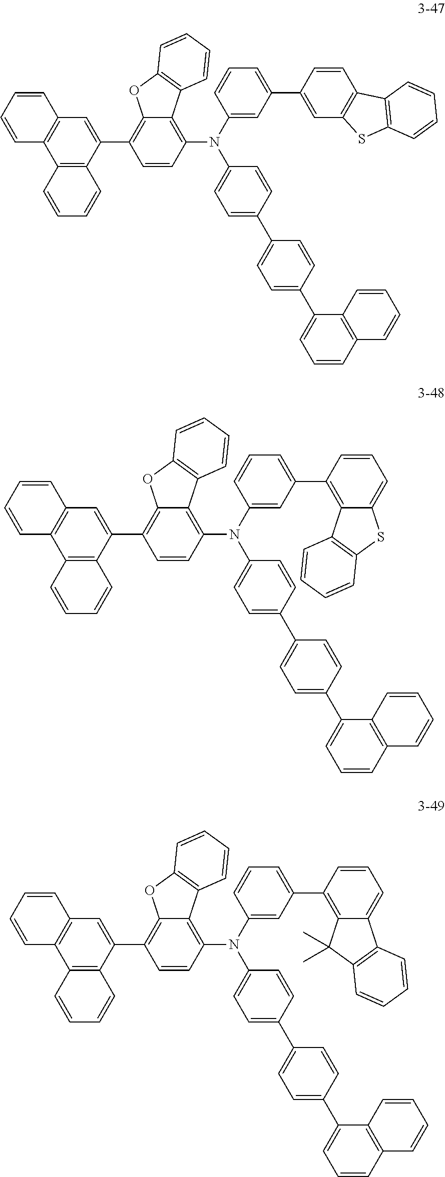 [SMILES: c1cc(-c2ccc3c(c2)sc2ccccc23)cc(N(c2ccc(-c3ccc(-c4cccc5ccccc45)cc3)cc2)c2ccc(-c3cc4ccccc4c4ccccc34)c3oc4ccccc4c23)c1 ; c1cc(-c2cccc3sc4ccccc4c23)cc(N(c2ccc(-c3ccc(-c4cccc5ccccc45)cc3)cc2)c2ccc(-c3cc4ccccc4c4ccccc34)c3oc4ccccc4c23)c1 ; CC1(C)c2ccccc2-c2cccc(-c3cccc(N(c4ccc(-c5ccc(-c6cccc7ccccc67)cc5)cc4)c4ccc(-c5cc6ccccc6c6ccccc56)c5oc6ccccc6c45)c3)c21]

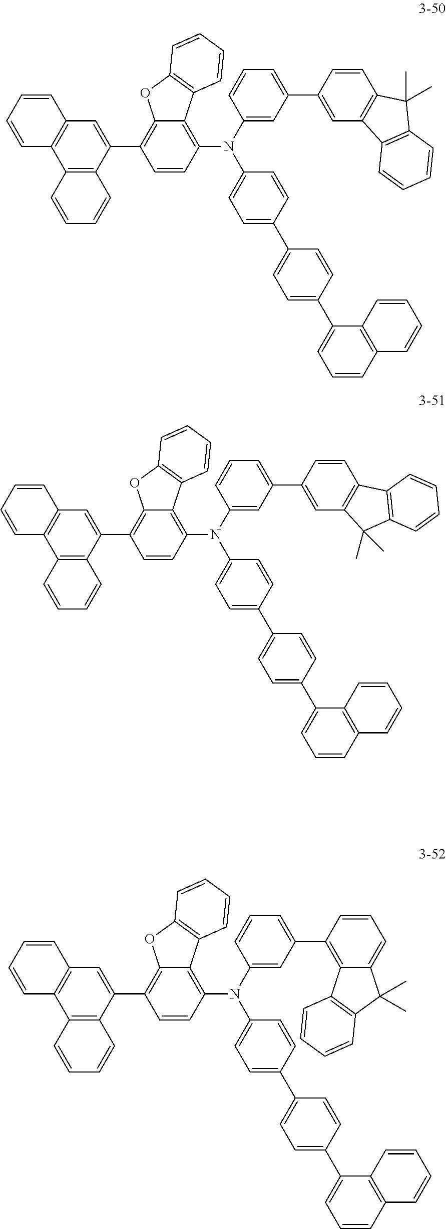 [SMILES: CC1(C)c2ccccc2-c2cc(-c3cccc(N(c4ccc(-c5ccc(-c6cccc7ccccc67)cc5)cc4)c4ccc(-c5cc6ccccc6c6ccccc56)c5oc6ccccc6c45)c3)ccc21 ; CC1(C)c2ccccc2-c2ccc(-c3cccc(N(c4ccc(-c5ccc(-c6cccc7ccccc67)cc5)cc4)c4ccc(-c5cc6ccccc6c6ccccc56)c5oc6ccccc6c45)c3)cc21 ; CC1(C)c2ccccc2-c2c(-c3cccc(N(c4ccc(-c5ccc(-c6cccc7ccccc67)cc5)cc4)c4ccc(-c5cc6ccccc6c6ccccc56)c5oc6ccccc6c45)c3)cccc21]

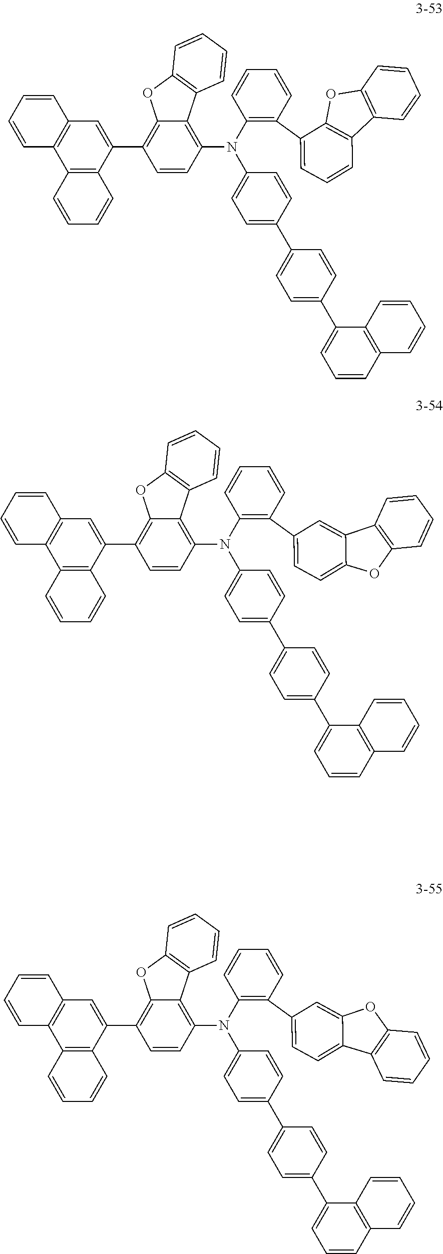 [SMILES: c1ccc(N(c2ccc(-c3ccc(-c4cccc5ccccc45)cc3)cc2)c2ccc(-c3cc4ccccc4c4ccccc34)c3oc4ccccc4c23)c(-c2cccc3c2oc2ccccc23)c1 ; c1ccc(N(c2ccc(-c3ccc(-c4cccc5ccccc45)cc3)cc2)c2ccc(-c3cc4ccccc4c4ccccc34)c3oc4ccccc4c23)c(-c2ccc3oc4ccccc4c3c2)c1 ; c1ccc(N(c2ccc(-c3ccc(-c4cccc5ccccc45)cc3)cc2)c2ccc(-c3cc4ccccc4c4ccccc34)c3oc4ccccc4c23)c(-c2ccc3c(c2)oc2ccccc23)c1]

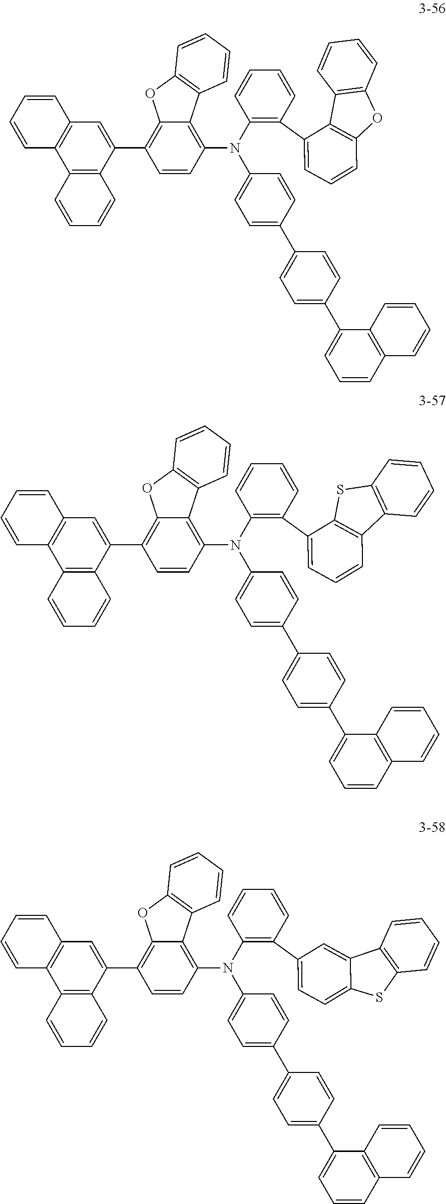 [SMILES: c1ccc(N(c2ccc(-c3ccc(-c4cccc5ccccc45)cc3)cc2)c2ccc(-c3cc4ccccc4c4ccccc34)c3oc4ccccc4c23)c(-c2cccc3oc4ccccc4c23)c1 ; c1ccc(N(c2ccc(-c3ccc(-c4cccc5ccccc45)cc3)cc2)c2ccc(-c3cc4ccccc4c4ccccc34)c3oc4ccccc4c23)c(-c2cccc3c2sc2ccccc23)c1 ; c1ccc(N(c2ccc(-c3ccc(-c4cccc5ccccc45)cc3)cc2)c2ccc(-c3cc4ccccc4c4ccccc34)c3oc4ccccc4c23)c(-c2ccc3sc4ccccc4c3c2)c1]

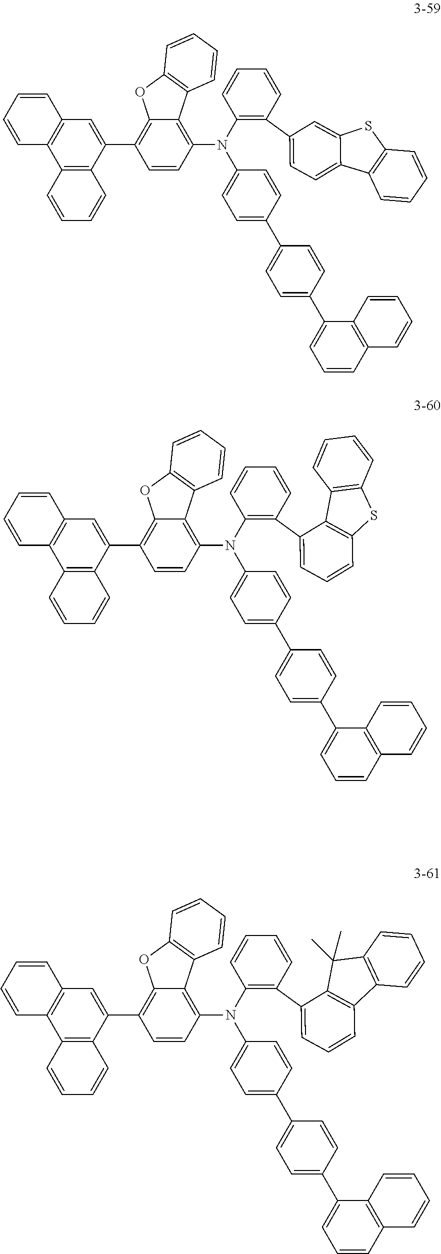 [SMILES: c1ccc(N(c2ccc(-c3ccc(-c4cccc5ccccc45)cc3)cc2)c2ccc(-c3cc4ccccc4c4ccccc34)c3oc4ccccc4c23)c(-c2ccc3c(c2)sc2ccccc23)c1 ; c1ccc(N(c2ccc(-c3ccc(-c4cccc5ccccc45)cc3)cc2)c2ccc(-c3cc4ccccc4c4ccccc34)c3oc4ccccc4c23)c(-c2cccc3sc4ccccc4c23)c1 ; CC1(C)c2ccccc2-c2cccc(-c3ccccc3N(c3ccc(-c4ccc(-c5cccc6ccccc56)cc4)cc3)c3ccc(-c4cc5ccccc5c5ccccc45)c4oc5ccccc5c34)c21]

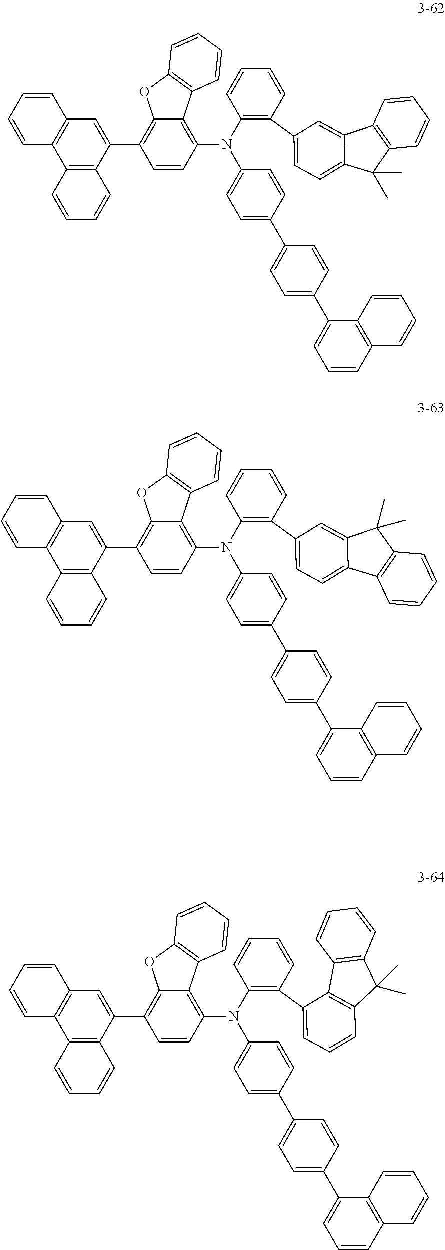 [SMILES: CC1(C)c2ccccc2-c2cc(-c3ccccc3N(c3ccc(-c4ccc(-c5cccc6ccccc56)cc4)cc3)c3ccc(-c4cc5ccccc5c5ccccc45)c4oc5ccccc5c34)ccc21 ; CC1(C)c2ccccc2-c2ccc(-c3ccccc3N(c3ccc(-c4ccc(-c5cccc6ccccc56)cc4)cc3)c3ccc(-c4cc5ccccc5c5ccccc45)c4oc5ccccc5c34)cc21 ; CC1(C)c2ccccc2-c2c(-c3ccccc3N(c3ccc(-c4ccc(-c5cccc6ccccc56)cc4)cc3)c3ccc(-c4cc5ccccc5c5ccccc45)c4oc5ccccc5c34)cccc21]

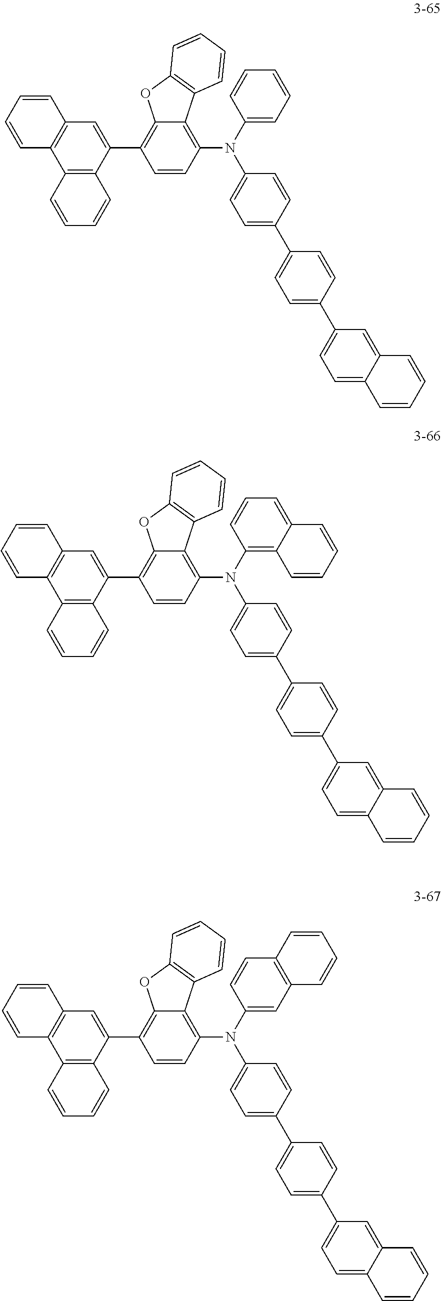 [SMILES: c1ccc(N(c2ccc(-c3ccc(-c4ccc5ccccc5c4)cc3)cc2)c2ccc(-c3cc4ccccc4c4ccccc34)c3oc4ccccc4c23)cc1 ; c1ccc2cc(-c3ccc(-c4ccc(N(c5cccc6ccccc56)c5ccc(-c6cc7ccccc7c7ccccc67)c6oc7ccccc7c56)cc4)cc3)ccc2c1 ; c1ccc2cc(-c3ccc(-c4ccc(N(c5ccc6ccccc6c5)c5ccc(-c6cc7ccccc7c7ccccc67)c6oc7ccccc7c56)cc4)cc3)ccc2c1]

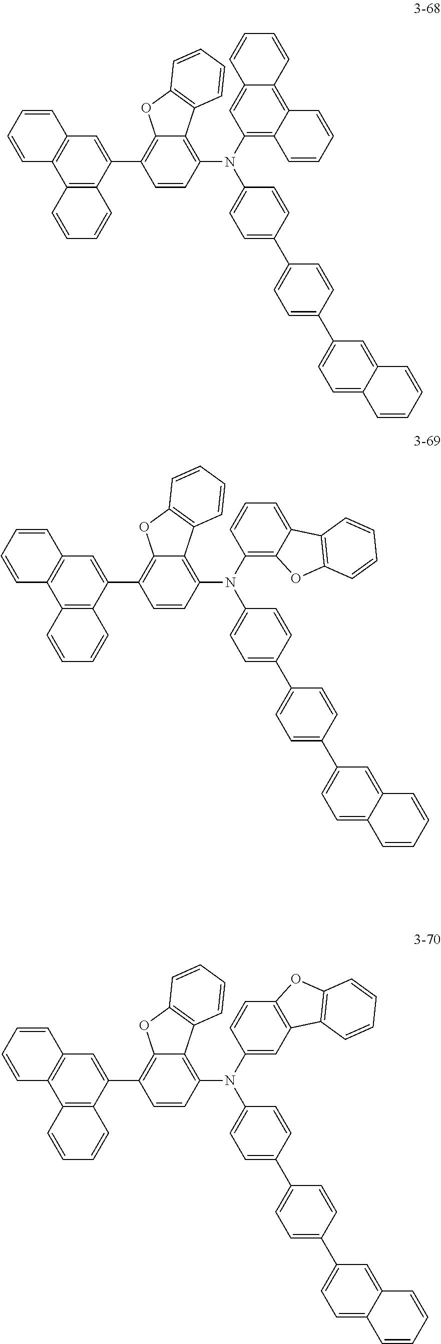 [SMILES: c1ccc2cc(-c3ccc(-c4ccc(N(c5cc6ccccc6c6ccccc56)c5ccc(-c6cc7ccccc7c7ccccc67)c6oc7ccccc7c56)cc4)cc3)ccc2c1 ; c1ccc2cc(-c3ccc(-c4ccc(N(c5cccc6c5oc5ccccc56)c5ccc(-c6cc7ccccc7c7ccccc67)c6oc7ccccc7c56)cc4)cc3)ccc2c1 ; c1ccc2cc(-c3ccc(-c4ccc(N(c5ccc6oc7ccccc7c6c5)c5ccc(-c6cc7ccccc7c7ccccc67)c6oc7ccccc7c56)cc4)cc3)ccc2c1]

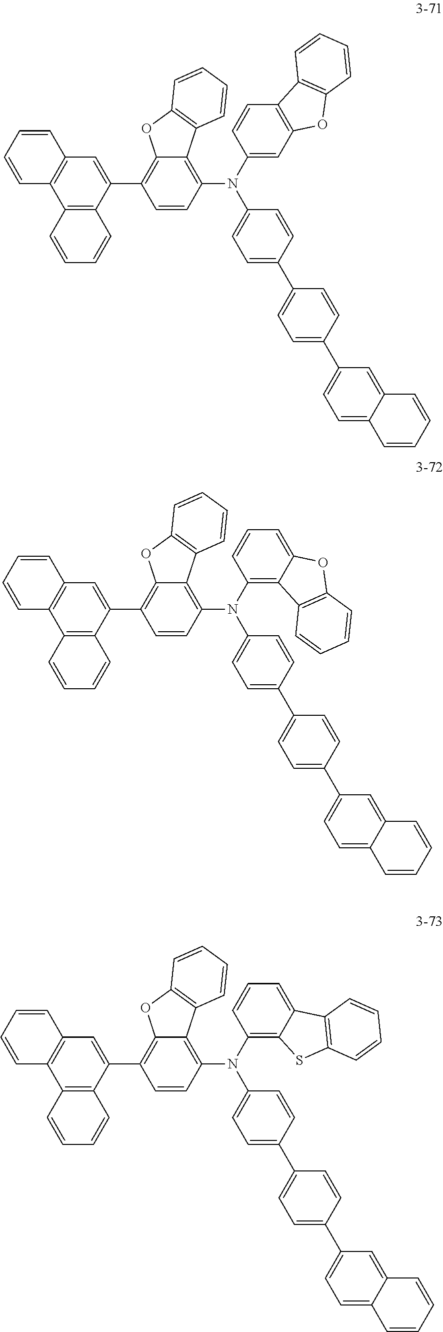 [SMILES: c1ccc2cc(-c3ccc(-c4ccc(N(c5ccc6c(c5)oc5ccccc56)c5ccc(-c6cc7ccccc7c7ccccc67)c6oc7ccccc7c56)cc4)cc3)ccc2c1 ; c1ccc2cc(-c3ccc(-c4ccc(N(c5cccc6oc7ccccc7c56)c5ccc(-c6cc7ccccc7c7ccccc67)c6oc7ccccc7c56)cc4)cc3)ccc2c1 ; c1ccc2cc(-c3ccc(-c4ccc(N(c5cccc6c5sc5ccccc56)c5ccc(-c6cc7ccccc7c7ccccc67)c6oc7ccccc7c56)cc4)cc3)ccc2c1]

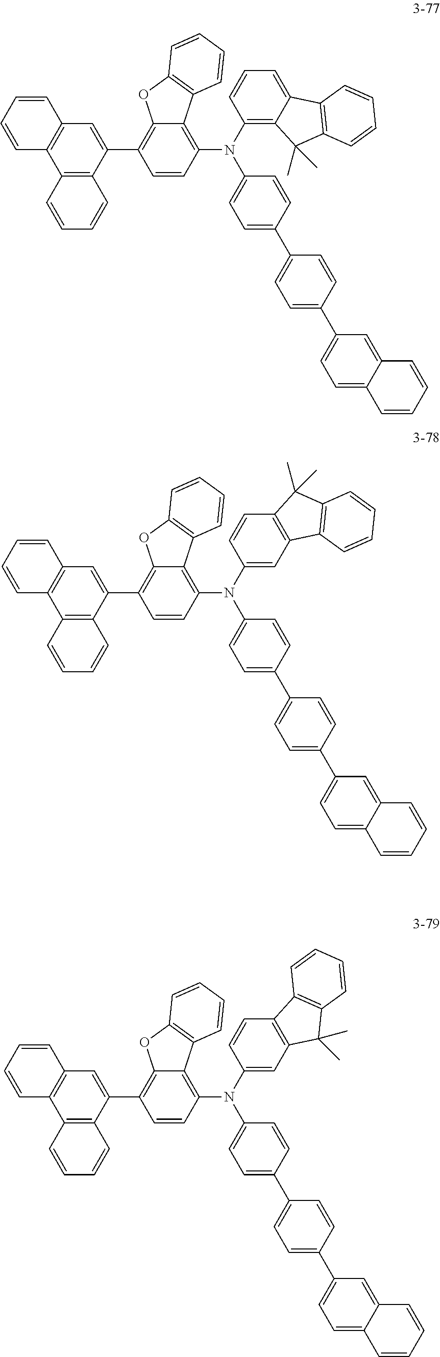 [SMILES: CC1(C)c2ccccc2-c2cccc(N(c3ccc(-c4ccc(-c5ccc6ccccc6c5)cc4)cc3)c3ccc(-c4cc5ccccc5c5ccccc45)c4oc5ccccc5c34)c21 ; CC1(C)c2ccccc2-c2cc(N(c3ccc(-c4ccc(-c5ccc6ccccc6c5)cc4)cc3)c3ccc(-c4cc5ccccc5c5ccccc45)c4oc5ccccc5c34)ccc21 ; CC1(C)c2ccccc2-c2ccc(N(c3ccc(-c4ccc(-c5ccc6ccccc6c5)cc4)cc3)c3ccc(-c4cc5ccccc5c5ccccc45)c4oc5ccccc5c34)cc21]

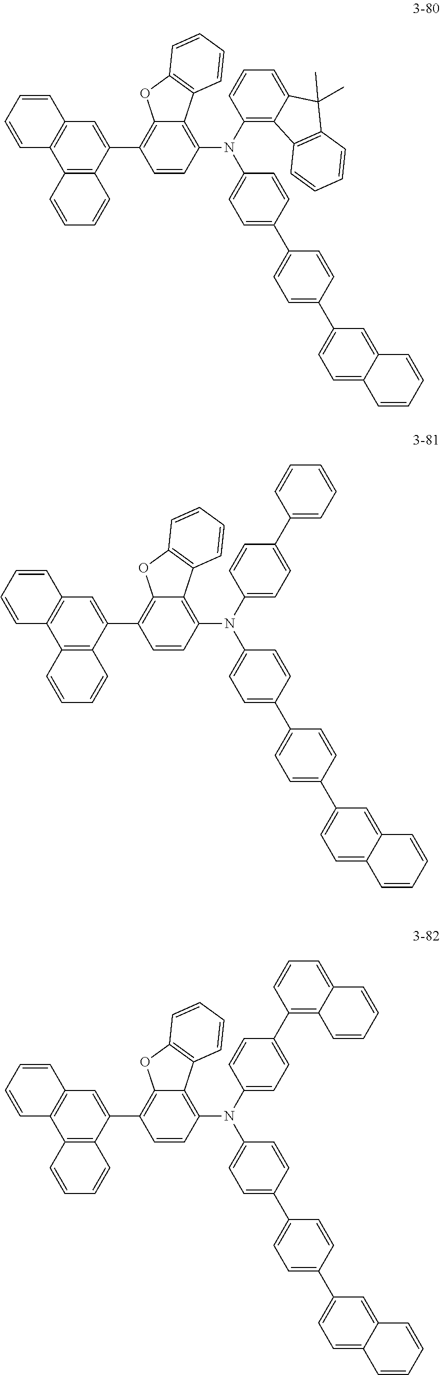 [SMILES: CC1(C)c2ccccc2-c2c(N(c3ccc(-c4ccc(-c5ccc6ccccc6c5)cc4)cc3)c3ccc(-c4cc5ccccc5c5ccccc45)c4oc5ccccc5c34)cccc21 ; c1ccc(-c2ccc(N(c3ccc(-c4ccc(-c5ccc6ccccc6c5)cc4)cc3)c3ccc(-c4cc5ccccc5c5ccccc45)c4oc5ccccc5c34)cc2)cc1 ; c1ccc2cc(-c3ccc(-c4ccc(N(c5ccc(-c6cccc7ccccc67)cc5)c5ccc(-c6cc7ccccc7c7ccccc67)c6oc7ccccc7c56)cc4)cc3)ccc2c1]

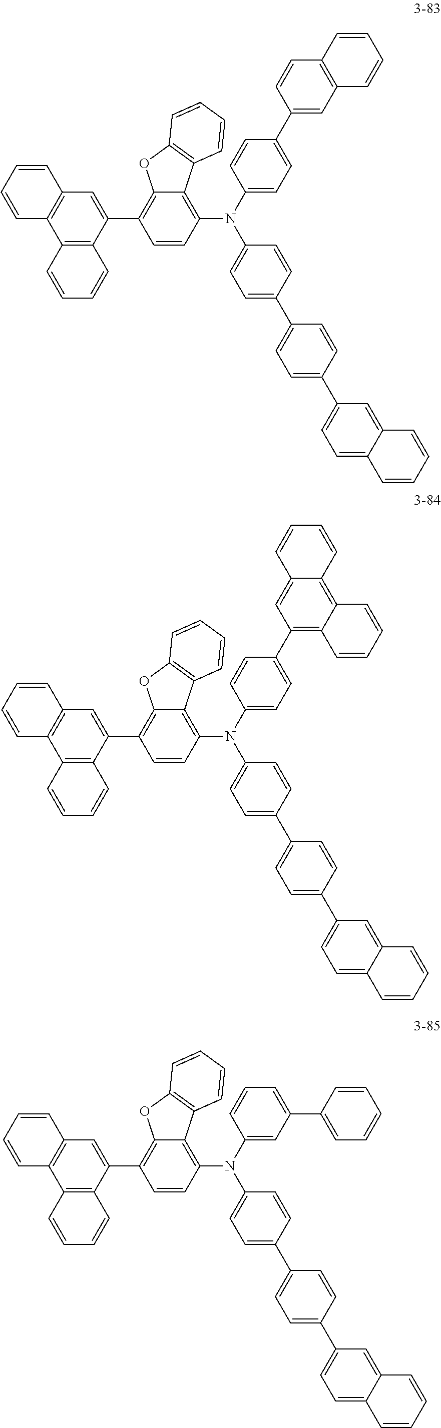 [SMILES: c1ccc2cc(-c3ccc(-c4ccc(N(c5ccc(-c6ccc7ccccc7c6)cc5)c5ccc(-c6cc7ccccc7c7ccccc67)c6oc7ccccc7c56)cc4)cc3)ccc2c1 ; c1ccc2cc(-c3ccc(-c4ccc(N(c5ccc(-c6cc7ccccc7c7ccccc67)cc5)c5ccc(-c6cc7ccccc7c7ccccc67)c6oc7ccccc7c56)cc4)cc3)ccc2c1 ; c1ccc(-c2cccc(N(c3ccc(-c4ccc(-c5ccc6ccccc6c5)cc4)cc3)c3ccc(-c4cc5ccccc5c5ccccc45)c4oc5ccccc5c34)c2)cc1]

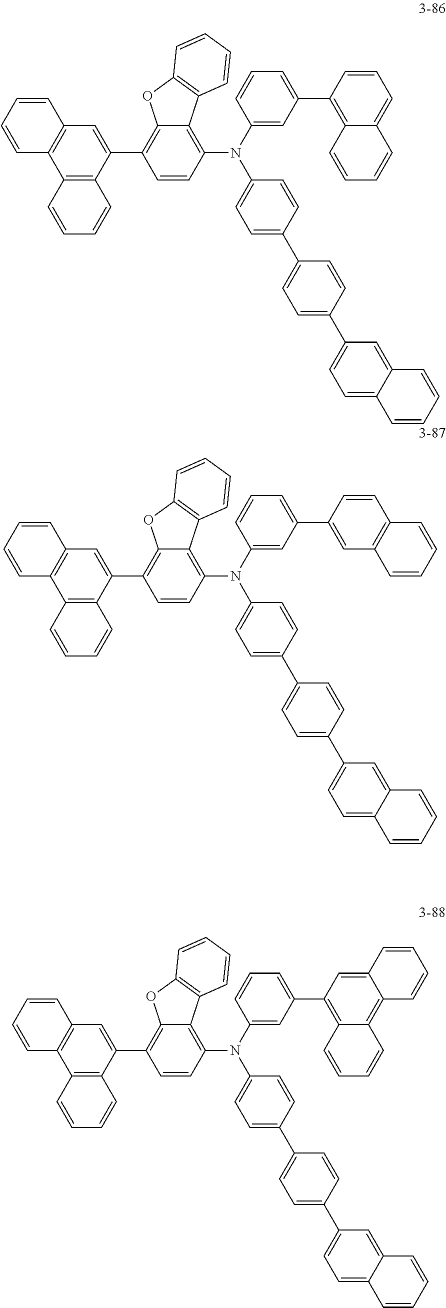 [SMILES: c1cc(-c2cccc3ccccc23)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2ccc3ccccc3c2)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2cc3ccccc3c3ccccc23)cc(N(c2ccc(-c3ccc(-c4ccc5ccccc5c4)cc3)cc2)c2ccc(-c3cc4ccccc4c4ccccc34)c3oc4ccccc4c23)c1]

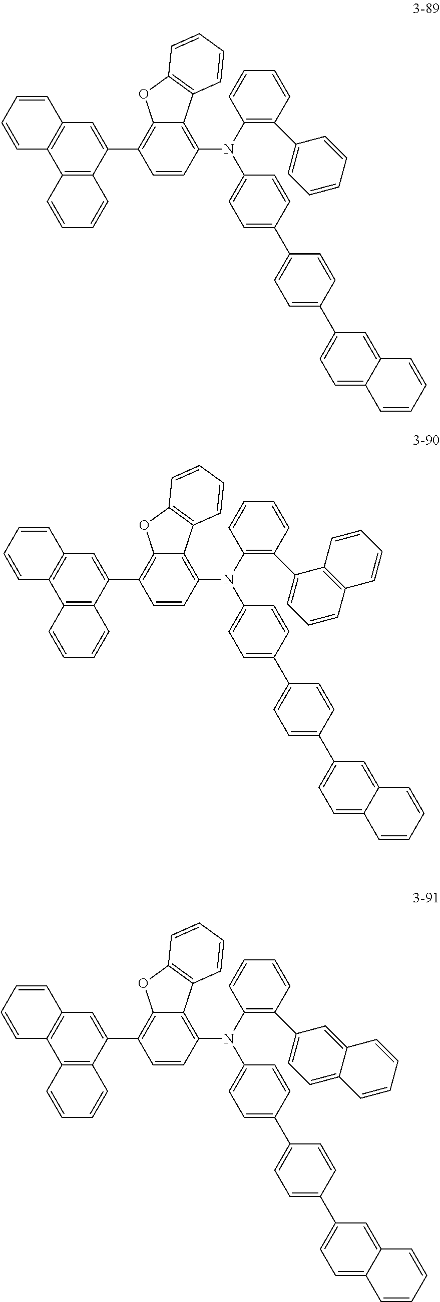 [SMILES: c1ccc(-c2ccccc2N(c2ccc(-c3ccc(-c4ccc5ccccc5c4)cc3)cc2)c2ccc(-c3cc4ccccc4c4ccccc34)c3oc4ccccc4c23)cc1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2cccc3ccccc23)c1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2ccc3ccccc3c2)c1]

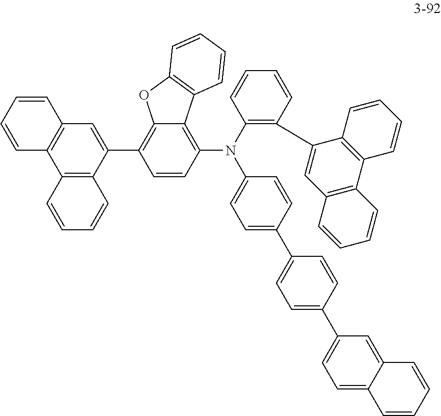 [SMILES: c1ccc(N(c2ccc(-c3ccc(-c4ccc5ccccc5c4)cc3)cc2)c2ccc(-c3cc4ccccc4c4ccccc34)c3oc4ccccc4c23)c(-c2cc3ccccc3c3ccccc23)c1]

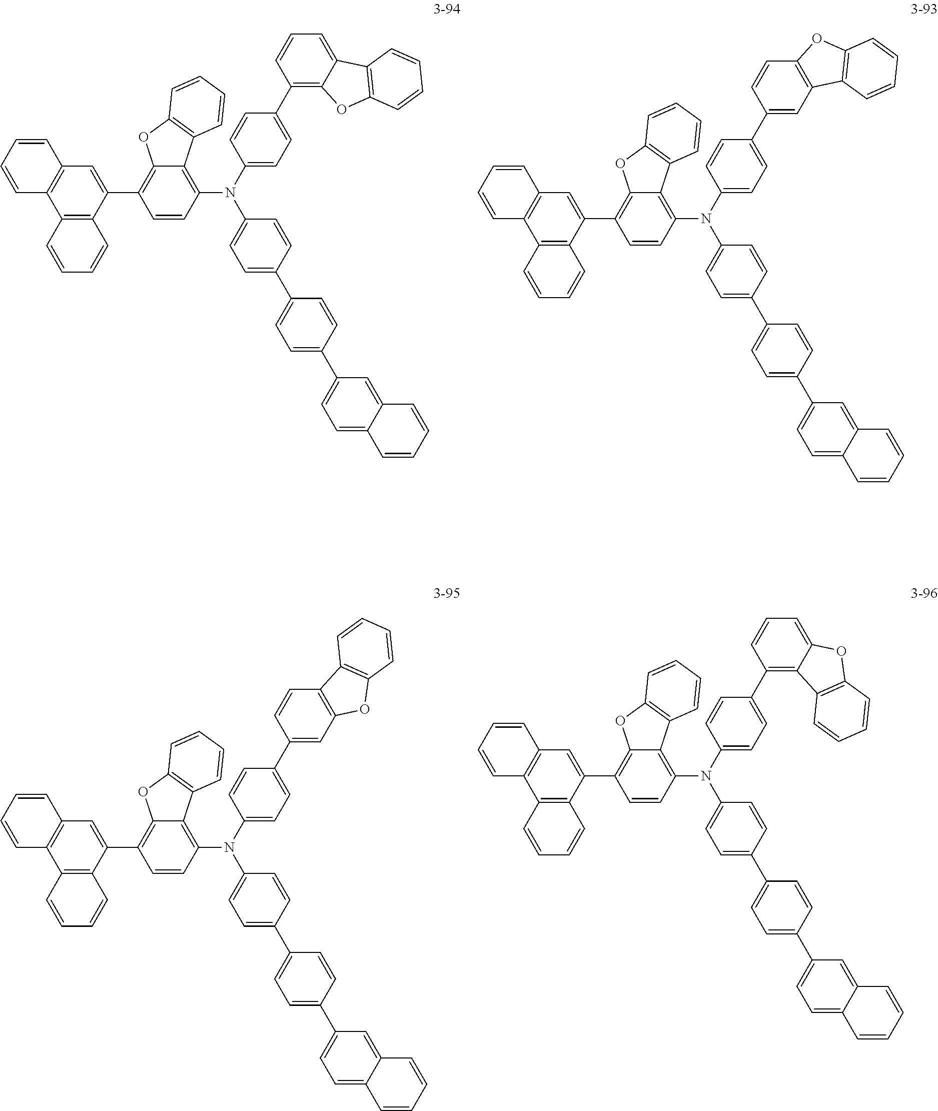 [SMILES: c1ccc2cc(-c3ccc(-c4ccc(N(c5ccc(-c6cccc7c6oc6ccccc67)cc5)c5ccc(-c6cc7ccccc7c7ccccc67)c6oc7ccccc7c56)cc4)cc3)ccc2c1 ; c1ccc2cc(-c3ccc(-c4ccc(N(c5ccc(-c6ccc7oc8ccccc8c7c6)cc5)c5ccc(-c6cc7ccccc7c7ccccc67)c6oc7ccccc7c56)cc4)cc3)ccc2c1 ; c1ccc2cc(-c3ccc(-c4ccc(N(c5ccc(-c6ccc7c(c6)oc6ccccc67)cc5)c5ccc(-c6cc7ccccc7c7ccccc67)c6oc7ccccc7c56)cc4)cc3)ccc2c1 ; c1ccc2cc(-c3ccc(-c4ccc(N(c5ccc(-c6cccc7oc8ccccc8c67)cc5)c5ccc(-c6cc7ccccc7c7ccccc67)c6oc7ccccc7c56)cc4)cc3)ccc2c1]

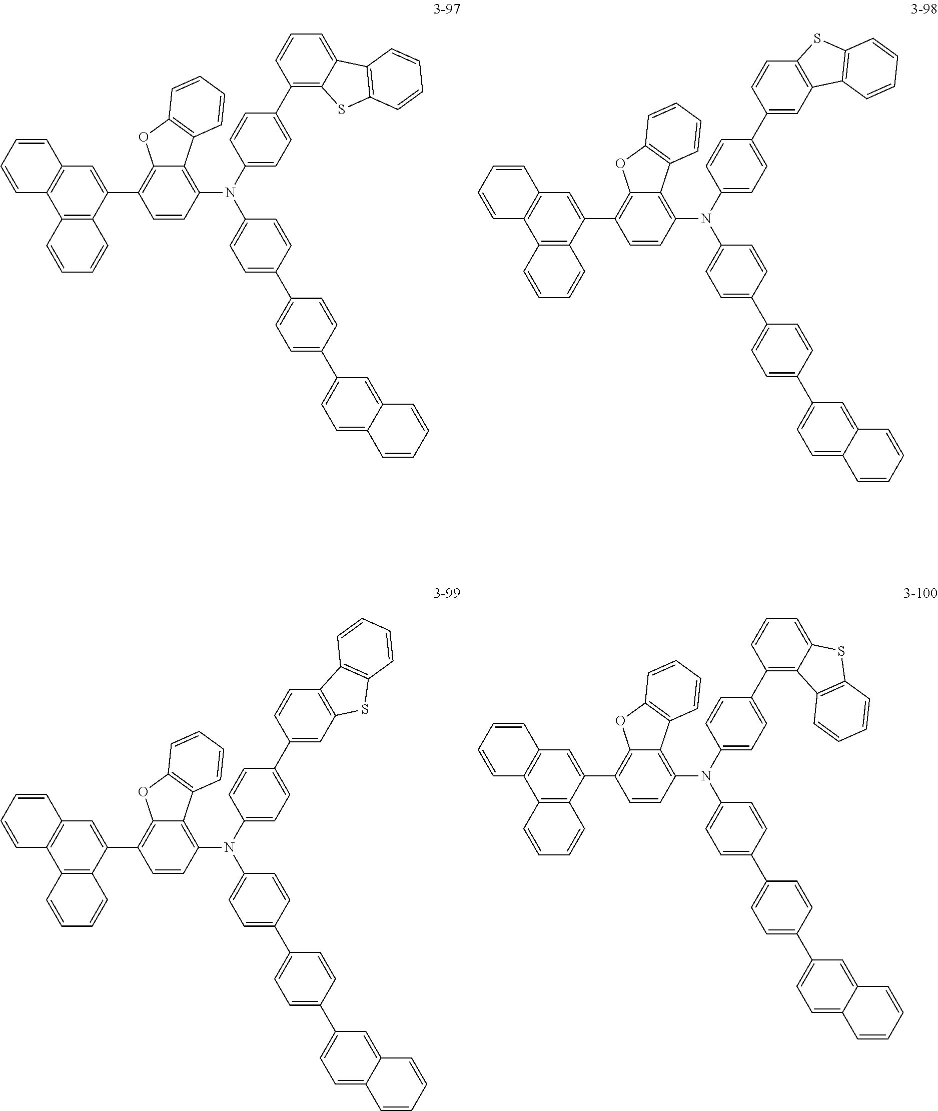 [SMILES: c1ccc2cc(-c3ccc(-c4ccc(N(c5ccc(-c6cccc7c6sc6ccccc67)cc5)c5ccc(-c6cc7ccccc7c7ccccc67)c6oc7ccccc7c56)cc4)cc3)ccc2c1 ; c1ccc2cc(-c3ccc(-c4ccc(N(c5ccc(-c6ccc7sc8ccccc8c7c6)cc5)c5ccc(-c6cc7ccccc7c7ccccc67)c6oc7ccccc7c56)cc4)cc3)ccc2c1 ; c1ccc2cc(-c3ccc(-c4ccc(N(c5ccc(-c6ccc7c(c6)sc6ccccc67)cc5)c5ccc(-c6cc7ccccc7c7ccccc67)c6oc7ccccc7c56)cc4)cc3)ccc2c1 ; c1ccc2cc(-c3ccc(-c4ccc(N(c5ccc(-c6cccc7sc8ccccc8c67)cc5)c5ccc(-c6cc7ccccc7c7ccccc67)c6oc7ccccc7c56)cc4)cc3)ccc2c1]

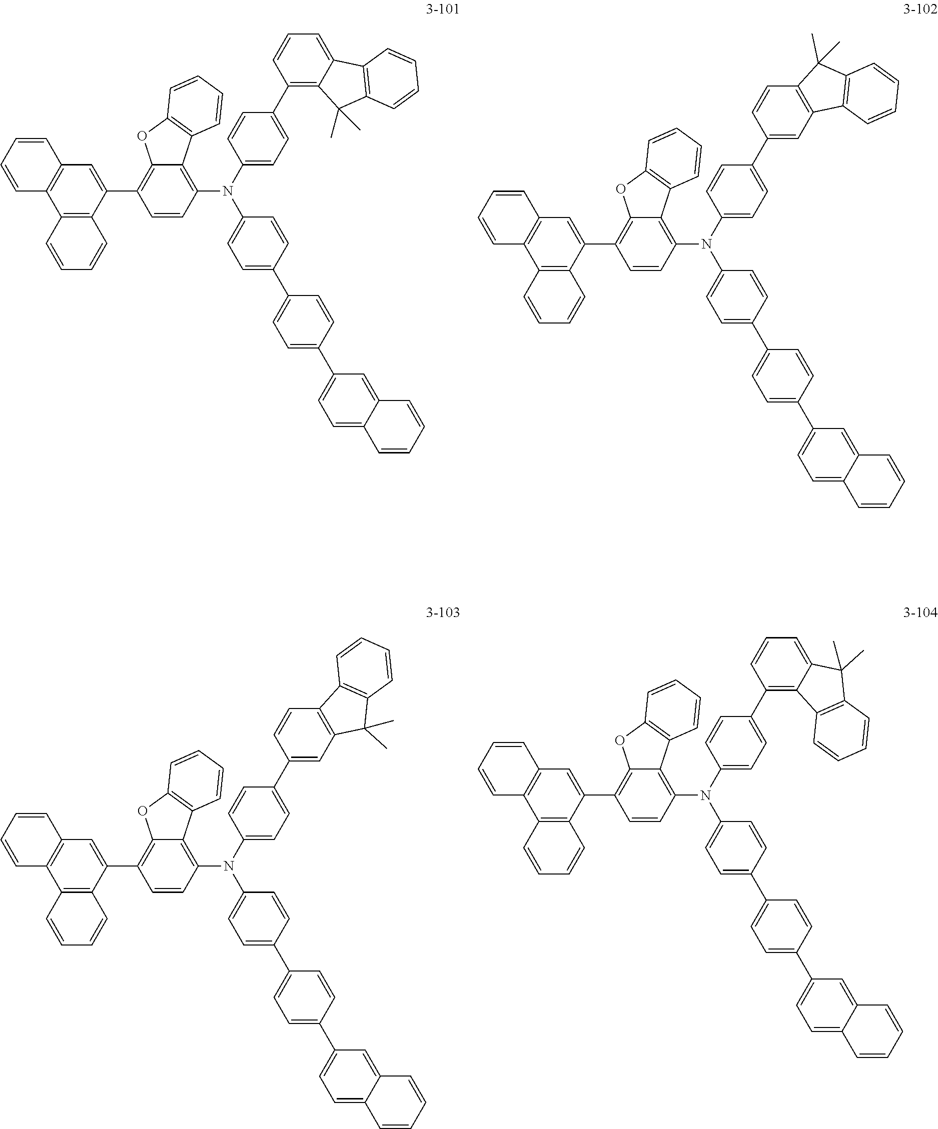 [SMILES: CC1(C)c2ccccc2-c2cccc(-c3ccc(N(c4ccc(-c5ccc(-c6ccc7ccccc7c6)cc5)cc4)c4ccc(-c5cc6ccccc6c6ccccc56)c5oc6ccccc6c45)cc3)c21 ; CC1(C)c2ccccc2-c2cc(-c3ccc(N(c4ccc(-c5ccc(-c6ccc7ccccc7c6)cc5)cc4)c4ccc(-c5cc6ccccc6c6ccccc56)c5oc6ccccc6c45)cc3)ccc21 ; CC1(C)c2ccccc2-c2ccc(-c3ccc(N(c4ccc(-c5ccc(-c6ccc7ccccc7c6)cc5)cc4)c4ccc(-c5cc6ccccc6c6ccccc56)c5oc6ccccc6c45)cc3)cc21 ; CC1(C)c2ccccc2-c2c(-c3ccc(N(c4ccc(-c5ccc(-c6ccc7ccccc7c6)cc5)cc4)c4ccc(-c5cc6ccccc6c6ccccc56)c5oc6ccccc6c45)cc3)cccc21]

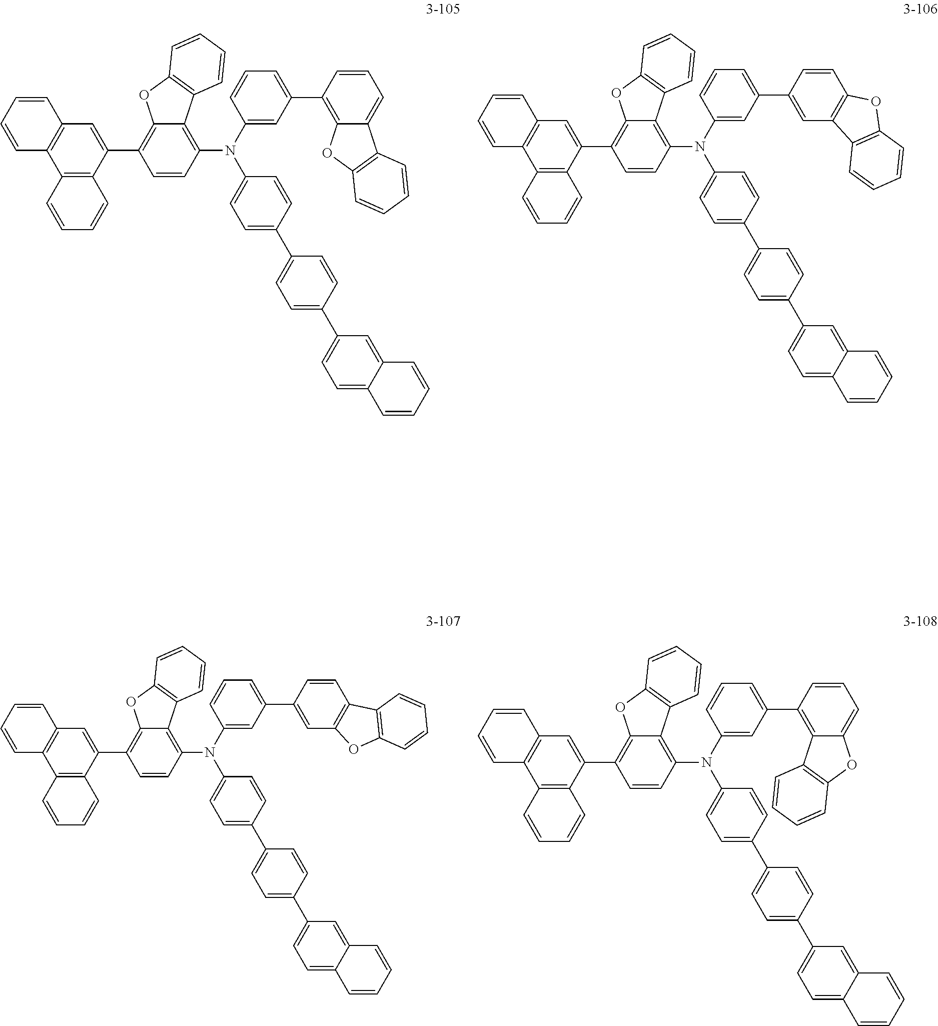 [SMILES: c1cc(-c2cccc3c2oc2ccccc23)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2ccc3oc4ccccc4c3c2)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2ccc3c(c2)oc2ccccc23)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2cccc3oc4ccccc4c23)cc(N(c2ccc(-c3ccc(-c4ccc5ccccc5c4)cc3)cc2)c2ccc(-c3cc4ccccc4c4ccccc34)c3oc4ccccc4c23)c1]

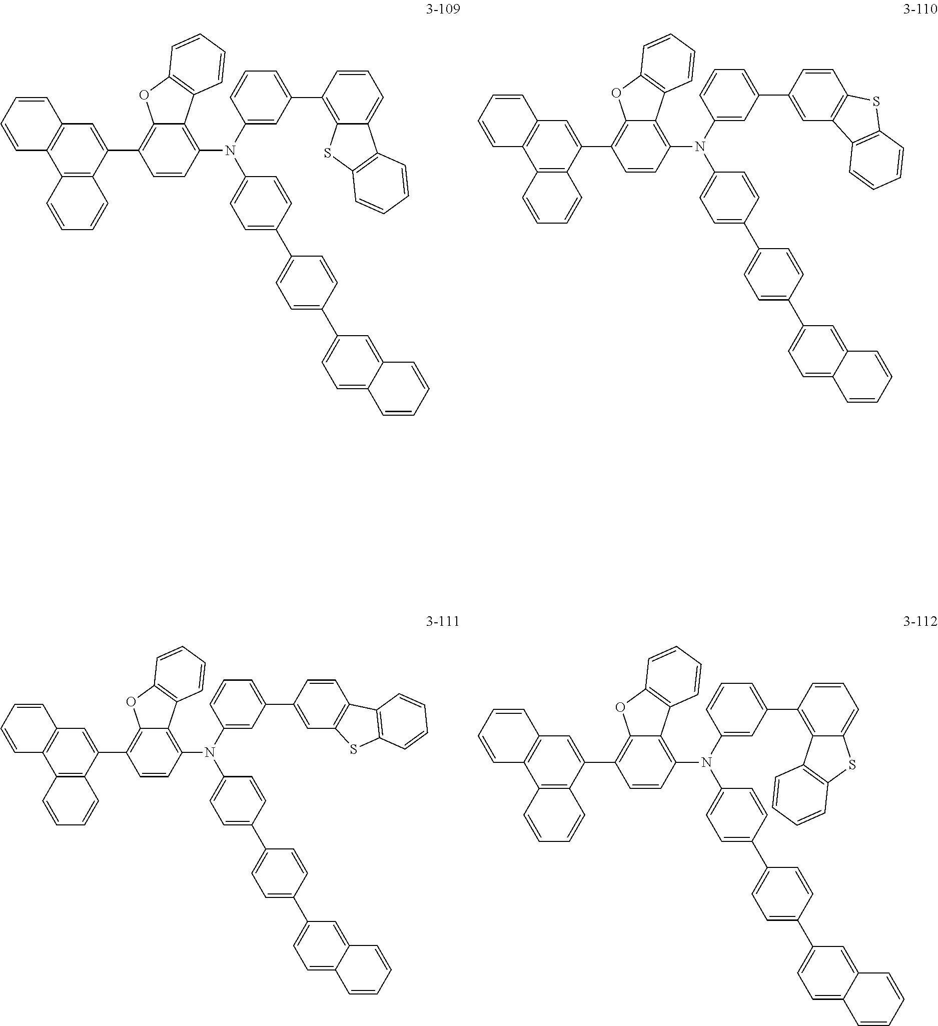 [SMILES: c1cc(-c2cccc3c2sc2ccccc23)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2ccc3sc4ccccc4c3c2)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2ccc3c(c2)sc2ccccc23)cc(N(c2ccc(-c3ccc(-c4ccc5ccccc5c4)cc3)cc2)c2ccc(-c3cc4ccccc4c4ccccc34)c3oc4ccccc4c23)c1 ; c1cc(-c2cccc3sc4ccccc4c23)cc(N(c2ccc(-c3ccc(-c4ccc5ccccc5c4)cc3)cc2)c2ccc(-c3cc4ccccc4c4ccccc34)c3oc4ccccc4c23)c1]

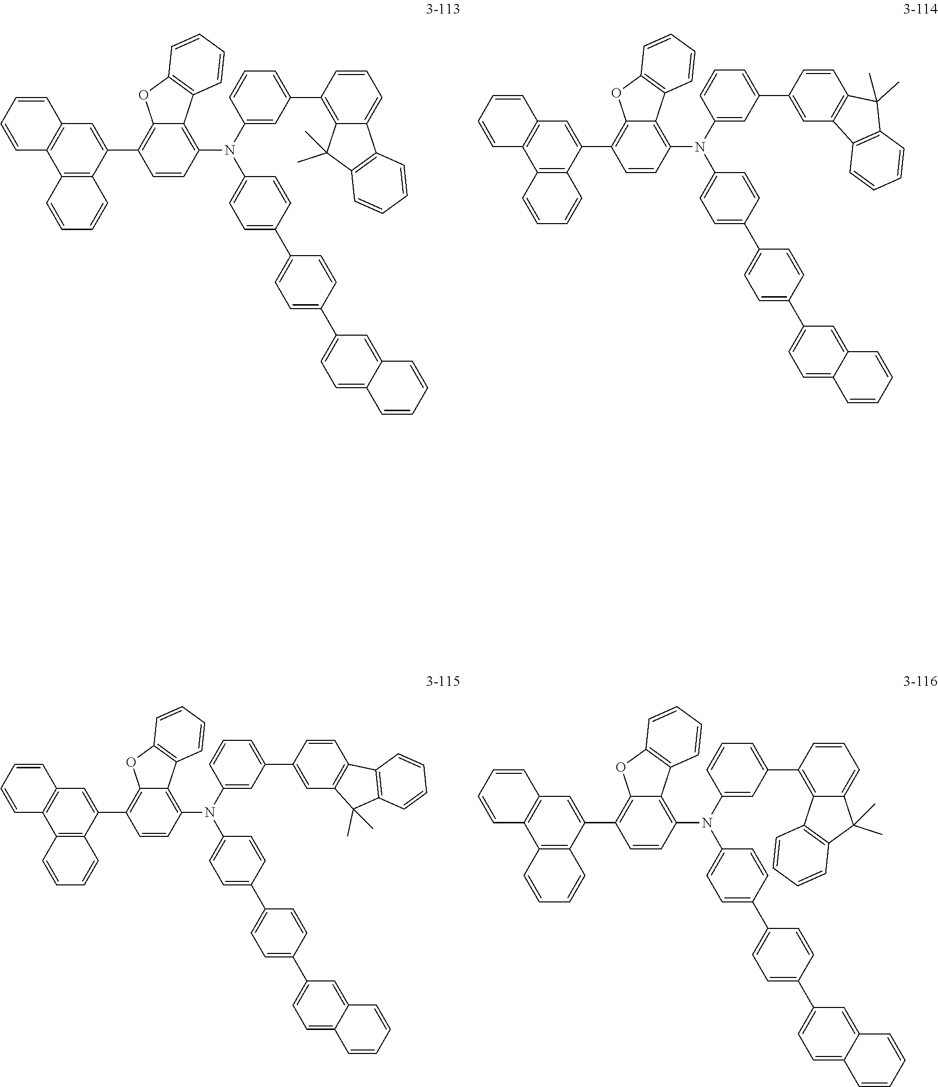 [SMILES: CC1(C)c2ccccc2-c2cccc(-c3cccc(N(c4ccc(-c5ccc(-c6ccc7ccccc7c6)cc5)cc4)c4ccc(-c5cc6ccccc6c6ccccc56)c5oc6ccccc6c45)c3)c21 ; CC1(C)c2ccccc2-c2cc(-c3cccc(N(c4ccc(-c5ccc(-c6ccc7ccccc7c6)cc5)cc4)c4ccc(-c5cc6ccccc6c6ccccc56)c5oc6ccccc6c45)c3)ccc21 ; CC1(C)c2ccccc2-c2ccc(-c3cccc(N(c4ccc(-c5ccc(-c6ccc7ccccc7c6)cc5)cc4)c4ccc(-c5cc6ccccc6c6ccccc56)c5oc6ccccc6c45)c3)cc21 ; CC1(C)c2ccccc2-c2c(-c3cccc(N(c4ccc(-c5ccc(-c6ccc7ccccc7c6)cc5)cc4)c4ccc(-c5cc6ccccc6c6ccccc56)c5oc6ccccc6c45)c3)cccc21]

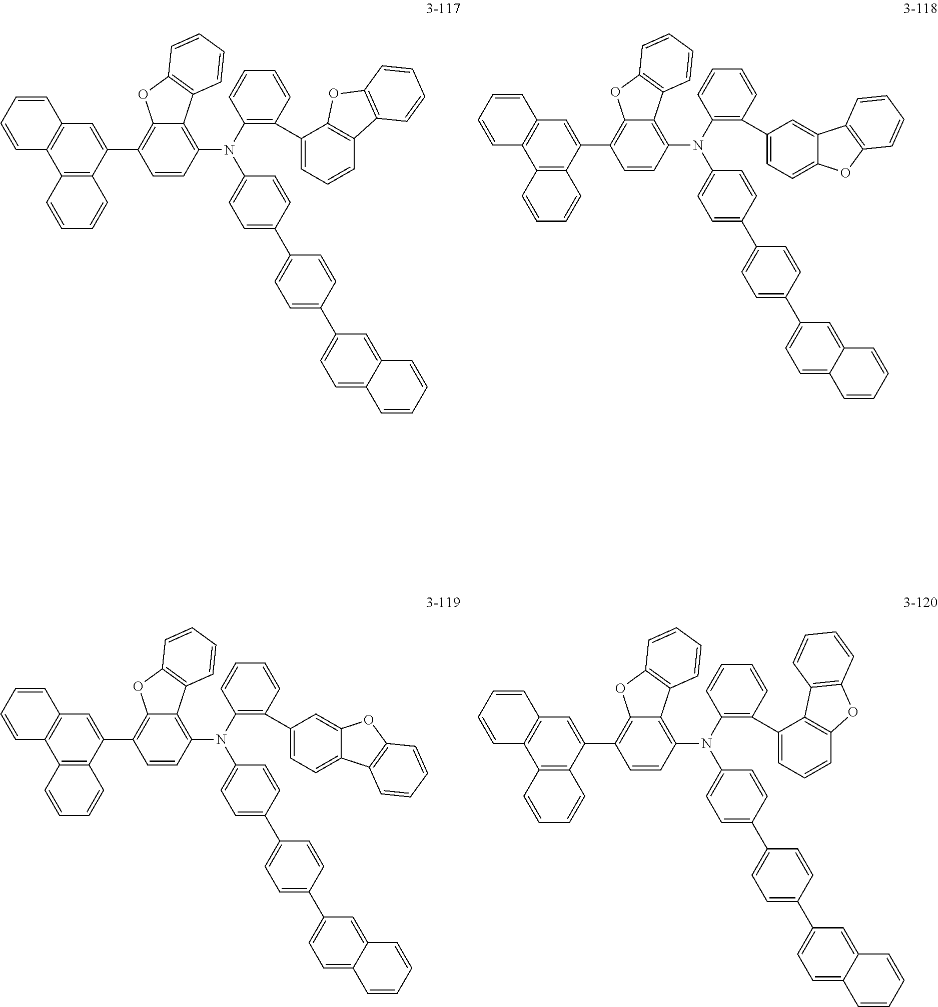 [SMILES: c1ccc(N(c2ccc(-c3ccc(-c4ccc5ccccc5c4)cc3)cc2)c2ccc(-c3cc4ccccc4c4ccccc34)c3oc4ccccc4c23)c(-c2cccc3c2oc2ccccc23)c1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2ccc3oc4ccccc4c3c2)c1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2ccc3c(c2)oc2ccccc23)c1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2cccc3oc4ccccc4c23)c1]

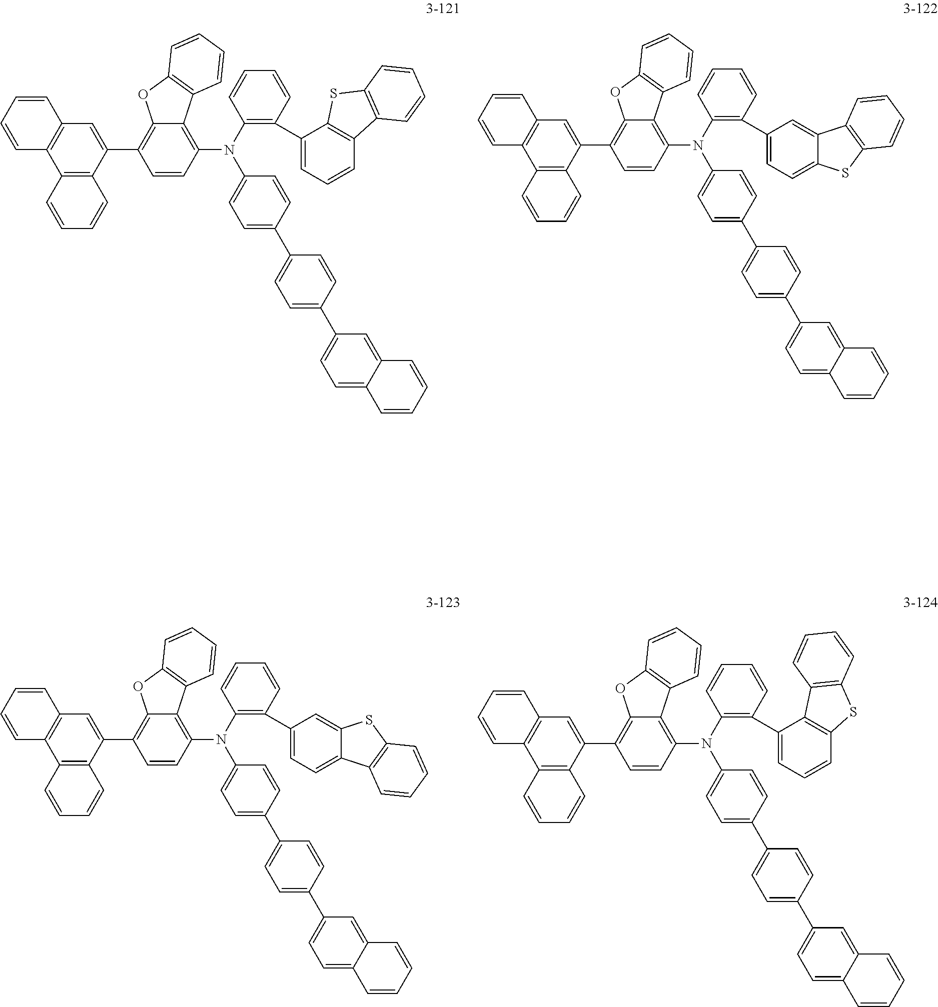 [SMILES: c1ccc(N(c2ccc(-c3ccc(-c4ccc5ccccc5c4)cc3)cc2)c2ccc(-c3cc4ccccc4c4ccccc34)c3oc4ccccc4c23)c(-c2cccc3c2sc2ccccc23)c1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2ccc3sc4ccccc4c3c2)c1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2ccc3c(c2)sc2ccccc23)c1 ; c1ccc(N(c2ccc(-c3ccc(-c4ccc5ccccc5c4)cc3)cc2)c2ccc(-c3cc4ccccc4c4ccccc34)c3oc4ccccc4c23)c(-c2cccc3sc4ccccc4c23)c1]

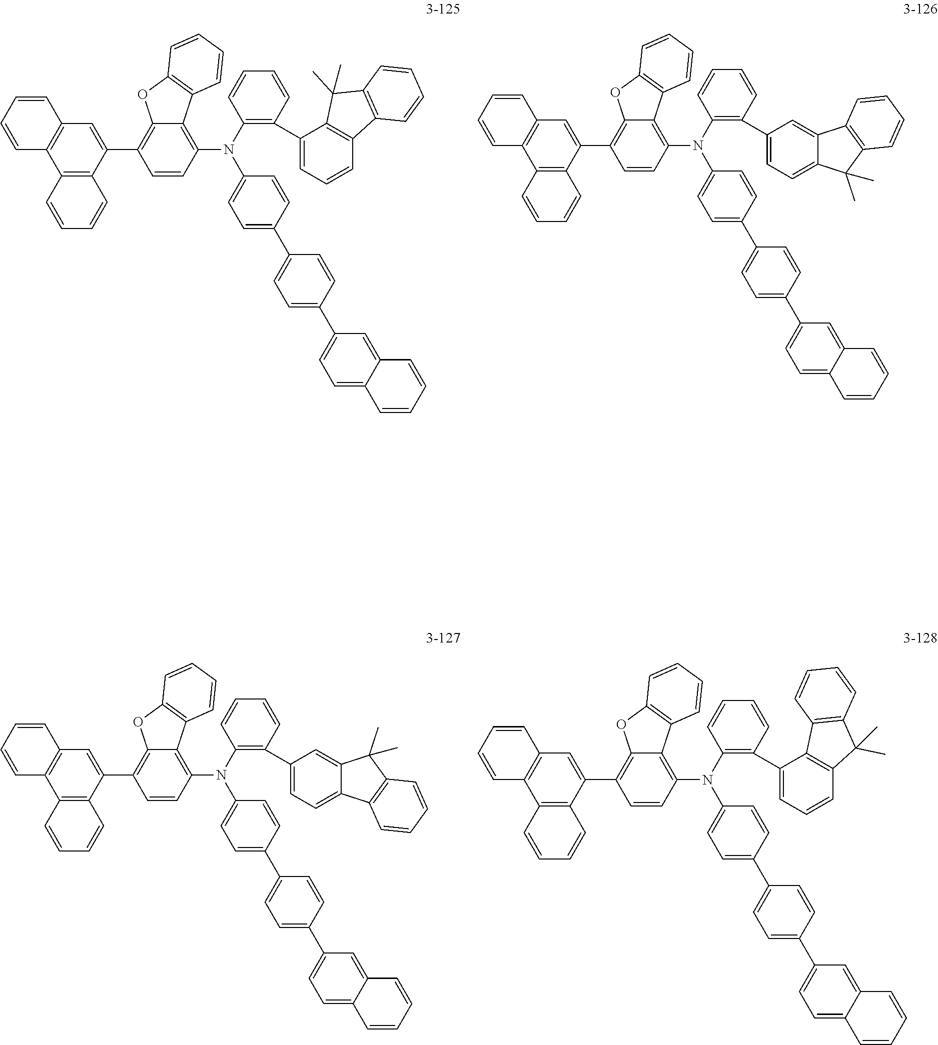 [SMILES: CC1(C)c2ccccc2-c2cccc(-c3ccccc3N(c3ccc(-c4ccc(-c5ccc6ccccc6c5)cc4)cc3)c3ccc(-c4cc5ccccc5c5ccccc45)c4oc5ccccc5c34)c21 ; CC1(C)c2ccccc2-c2cc(-c3ccccc3N(c3ccc(-c4ccc(-c5ccc6ccccc6c5)cc4)cc3)c3ccc(-c4cc5ccccc5c5ccccc45)c4oc5ccccc5c34)ccc21 ; CC1(C)c2ccccc2-c2ccc(-c3ccccc3N(c3ccc(-c4ccc(-c5ccc6ccccc6c5)cc4)cc3)c3ccc(-c4cc5ccccc5c5ccccc45)c4oc5ccccc5c34)cc21 ; CC1(C)c2ccccc2-c2c(-c3ccccc3N(c3ccc(-c4ccc(-c5ccc6ccccc6c5)cc4)cc3)c3ccc(-c4cc5ccccc5c5ccccc45)c4oc5ccccc5c34)cccc21]

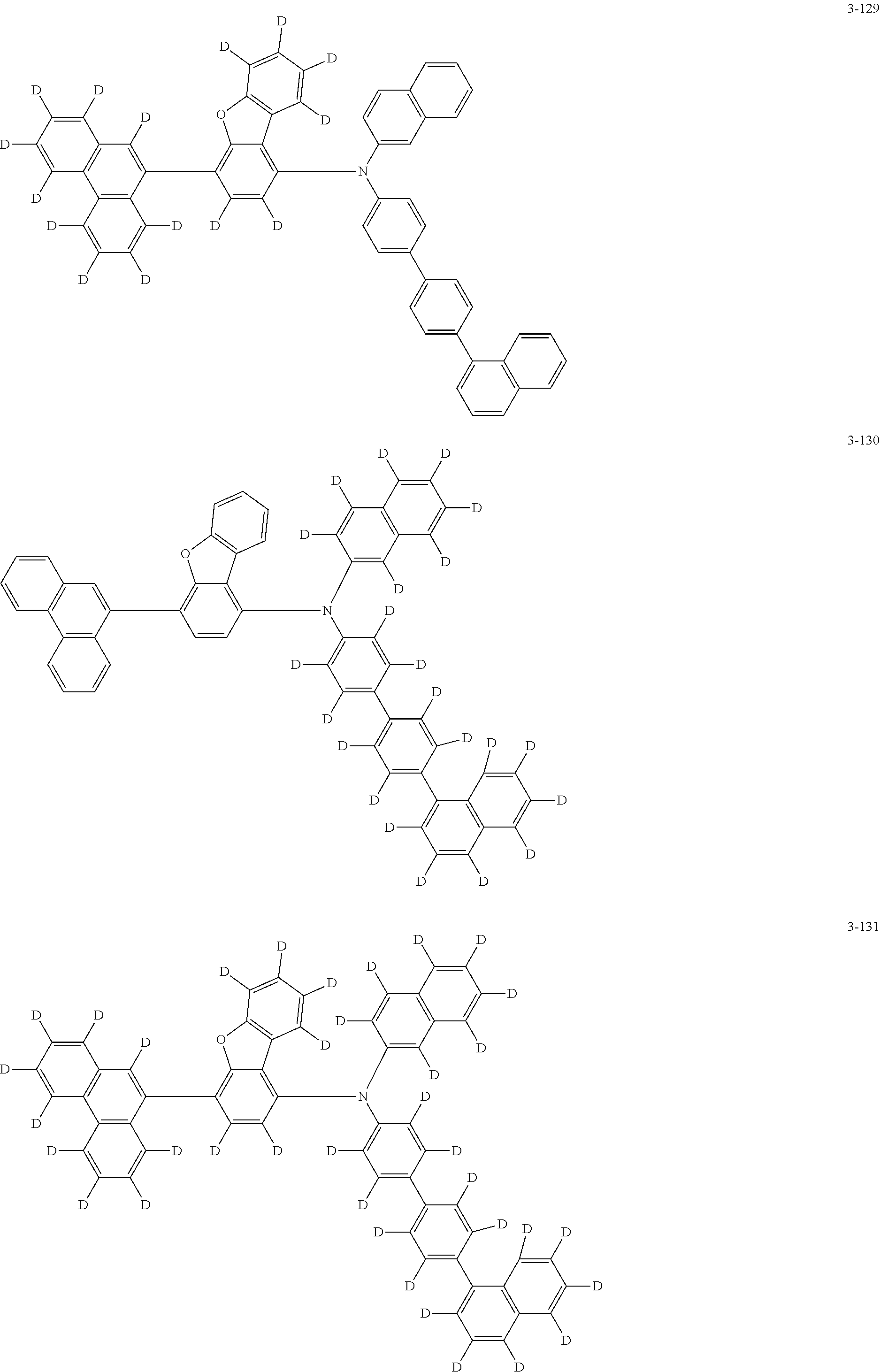

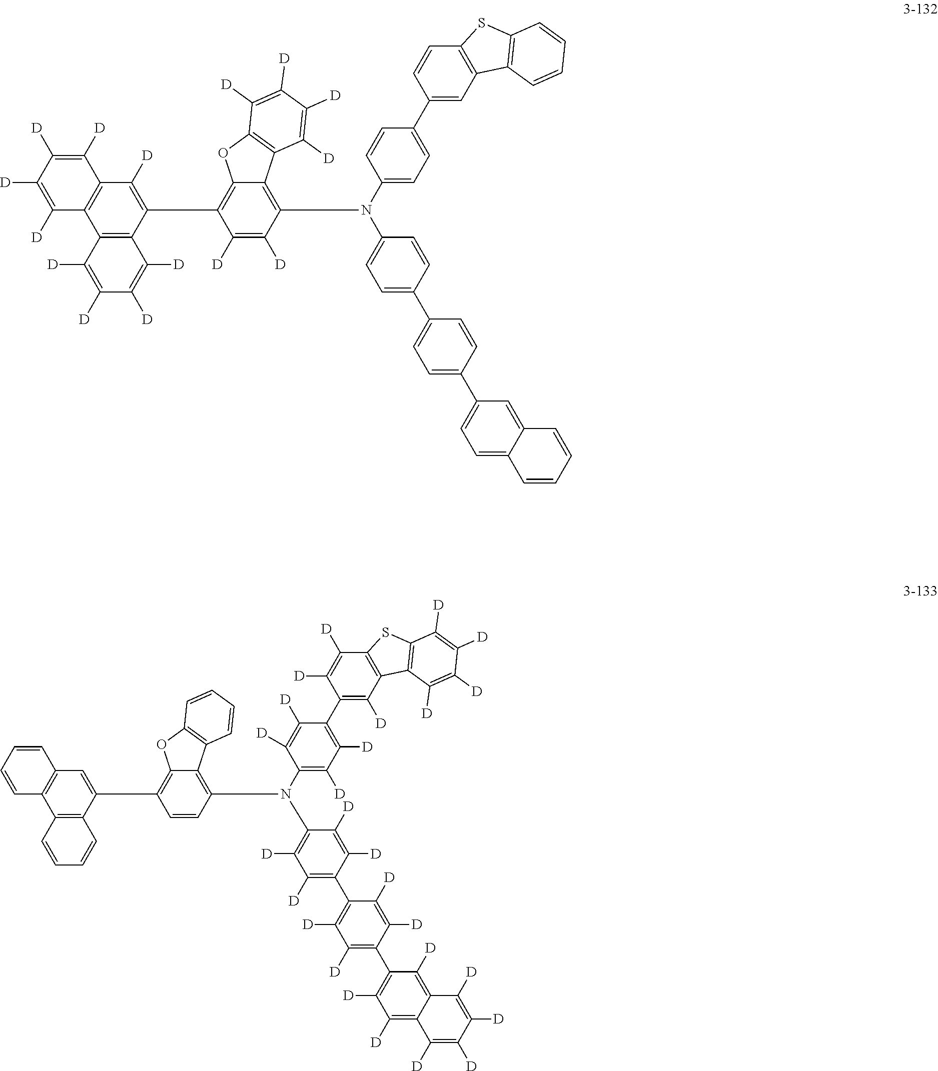 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(N(c4ccc(-c5ccc(-c6ccc7ccccc7c6)cc5)cc4)c4ccc(-c5ccc6sc7ccccc7c6c5)cc4)c32)c1[2H] ; [2H]c1c([2H])c(-c2c([2H])c([2H])c3c([2H])c([2H])c([2H])c([2H])c3c2[2H])c([2H])c([2H])c1-c1c([2H])c([2H])c(N(c2c([2H])c([2H])c(-c3c([2H])c([2H])c4sc5c([2H])c([2H])c([2H])c([2H])c5c4c3[2H])c([2H])c2[2H])c2ccc(-c3cc4ccccc4c4ccccc34)c3oc4ccccc4c23)c([2H])c1[2H]]

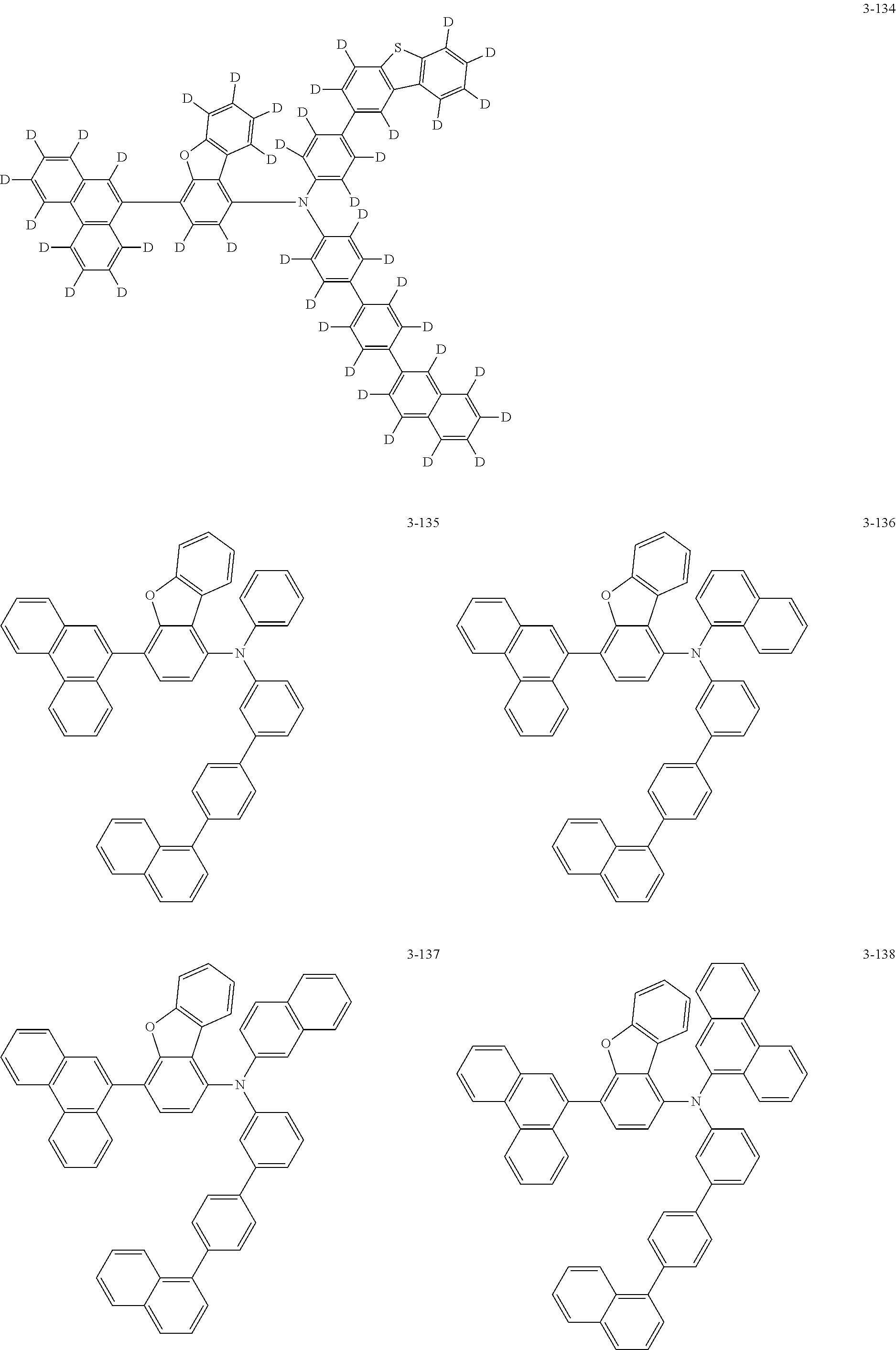

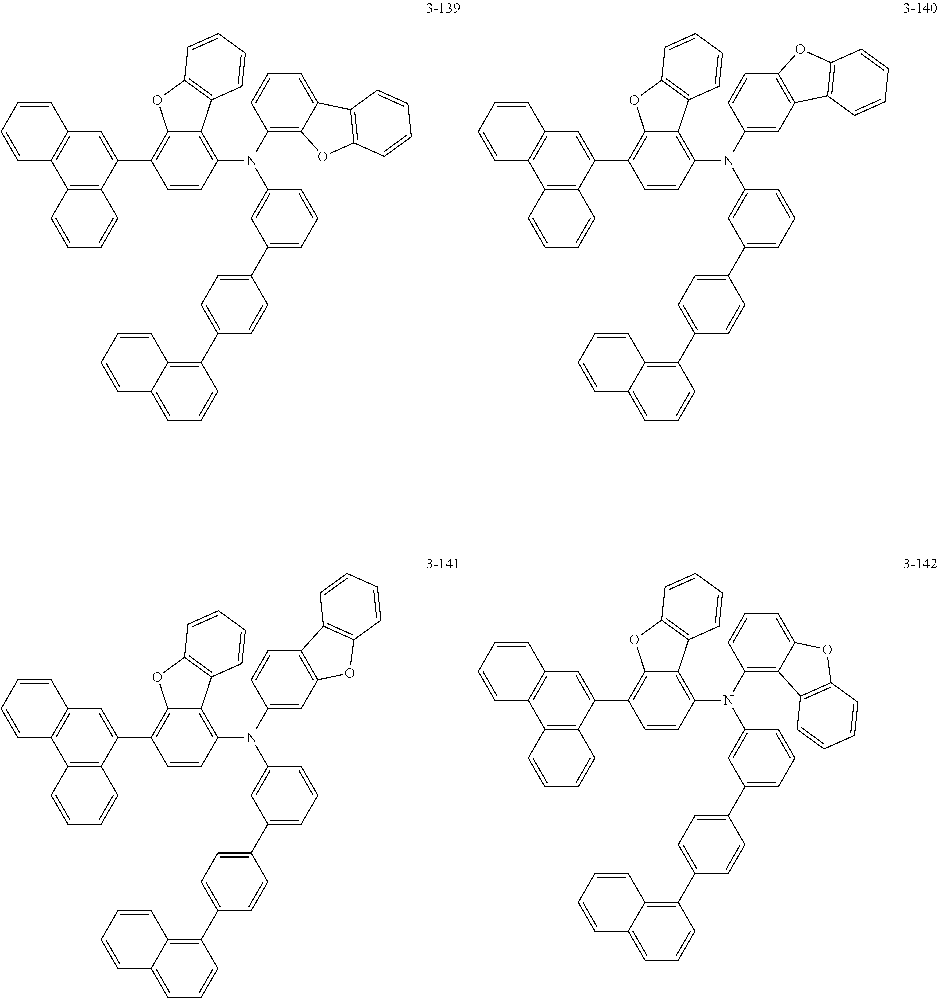 [SMILES: c1cc(-c2ccc(-c3cccc4ccccc34)cc2)cc(N(c2cccc3c2oc2ccccc23)c2ccc(-c3cc4ccccc4c4ccccc34)c3oc4ccccc4c23)c1 ; c1cc(-c2ccc(-c3cccc4ccccc34)cc2)cc(N(c2ccc3oc4ccccc4c3c2)c2ccc(-c3cc4ccccc4c4ccccc34)c3oc4ccccc4c23)c1 ; c1cc(-c2ccc(-c3cccc4ccccc34)cc2)cc(N(c2ccc3c(c2)oc2ccccc23)c2ccc(-c3cc4ccccc4c4ccccc34)c3oc4ccccc4c23)c1 ; c1cc(-c2ccc(-c3cccc4ccccc34)cc2)cc(N(c2cccc3oc4ccccc4c23)c2ccc(-c3cc4ccccc4c4ccccc34)c3oc4ccccc4c23)c1]

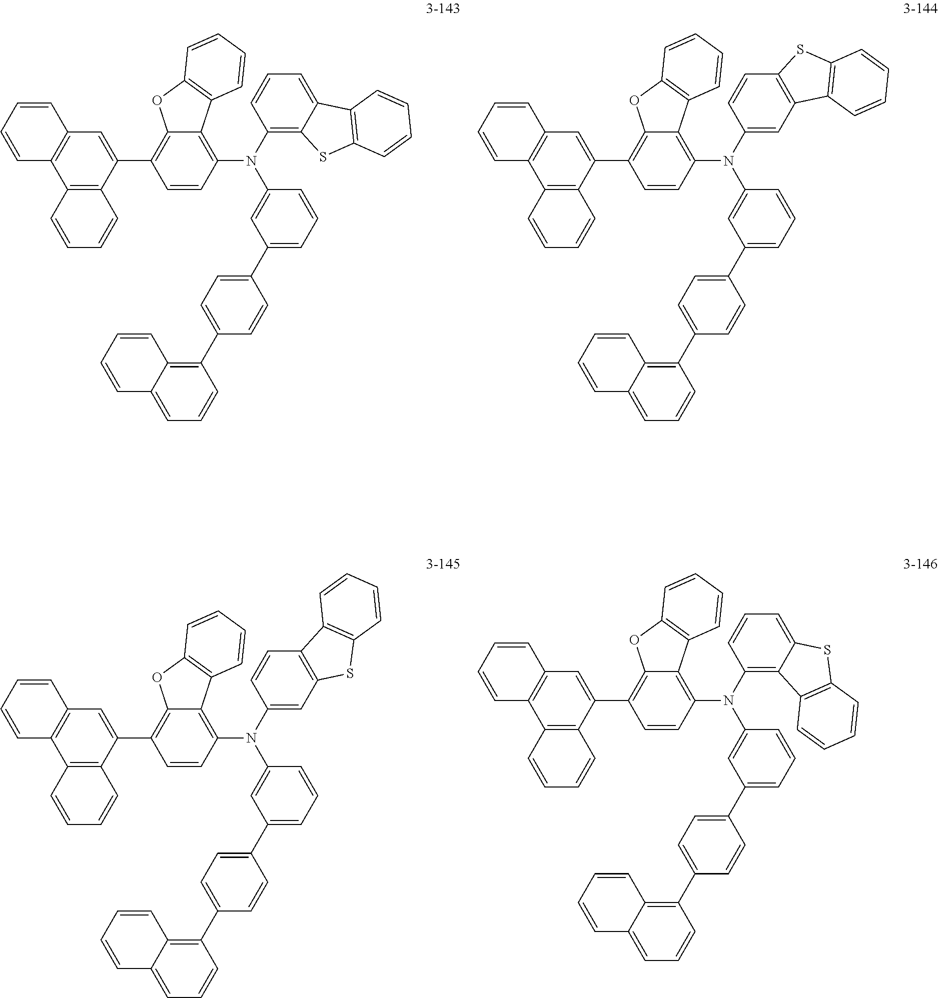 [SMILES: c1cc(-c2ccc(-c3cccc4ccccc34)cc2)cc(N(c2cccc3c2sc2ccccc23)c2ccc(-c3cc4ccccc4c4ccccc34)c3oc4ccccc4c23)c1 ; c1cc(-c2ccc(-c3cccc4ccccc34)cc2)cc(N(c2ccc3sc4ccccc4c3c2)c2ccc(-c3cc4ccccc4c4ccccc34)c3oc4ccccc4c23)c1 ; c1cc(-c2ccc(-c3cccc4ccccc34)cc2)cc(N(c2ccc3c(c2)sc2ccccc23)c2ccc(-c3cc4ccccc4c4ccccc34)c3oc4ccccc4c23)c1 ; c1cc(-c2ccc(-c3cccc4ccccc34)cc2)cc(N(c2cccc3sc4ccccc4c23)c2ccc(-c3cc4ccccc4c4ccccc34)c3oc4ccccc4c23)c1]

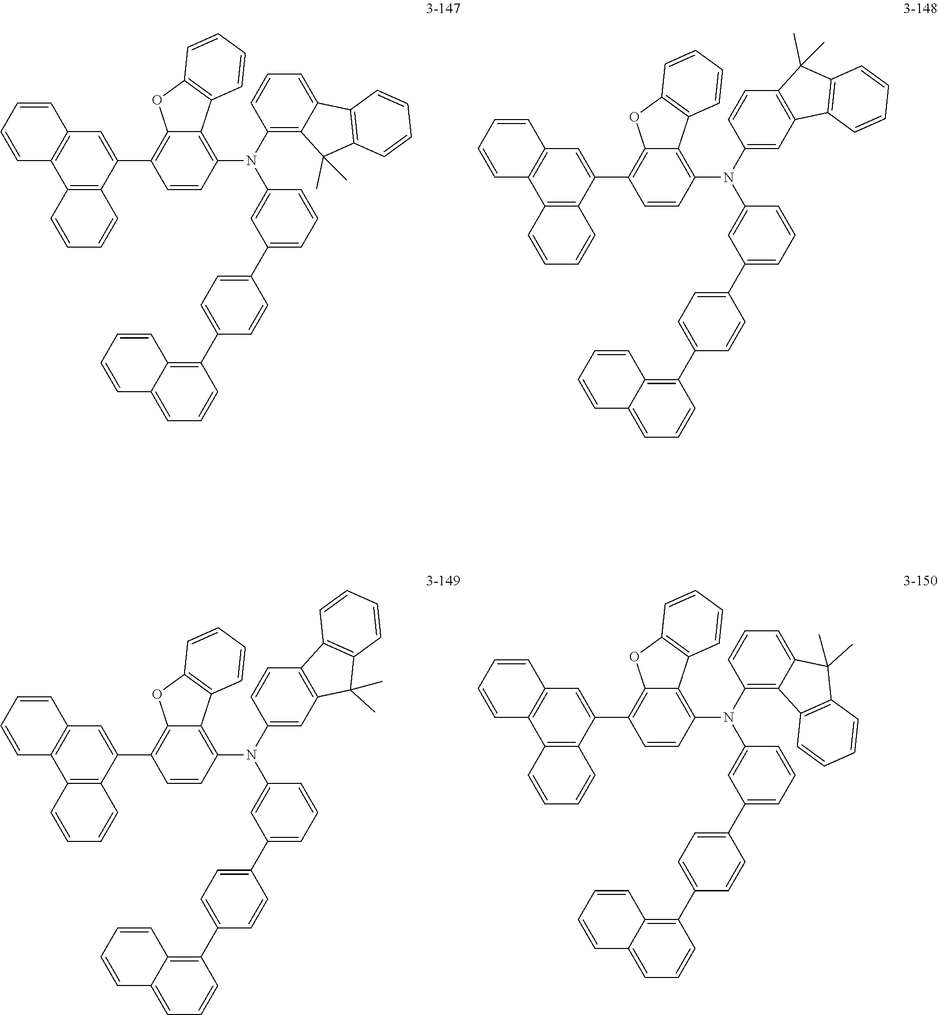 [SMILES: CC1(C)c2ccccc2-c2cccc(N(c3cccc(-c4ccc(-c5cccc6ccccc56)cc4)c3)c3ccc(-c4cc5ccccc5c5ccccc45)c4oc5ccccc5c34)c21 ; CC1(C)c2ccccc2-c2cc(N(c3cccc(-c4ccc(-c5cccc6ccccc56)cc4)c3)c3ccc(-c4cc5ccccc5c5ccccc45)c4oc5ccccc5c34)ccc21 ; CC1(C)c2ccccc2-c2ccc(N(c3cccc(-c4ccc(-c5cccc6ccccc56)cc4)c3)c3ccc(-c4cc5ccccc5c5ccccc45)c4oc5ccccc5c34)cc21 ; CC1(C)c2ccccc2-c2c(N(c3cccc(-c4ccc(-c5cccc6ccccc56)cc4)c3)c3ccc(-c4cc5ccccc5c5ccccc45)c4oc5ccccc5c34)cccc21]

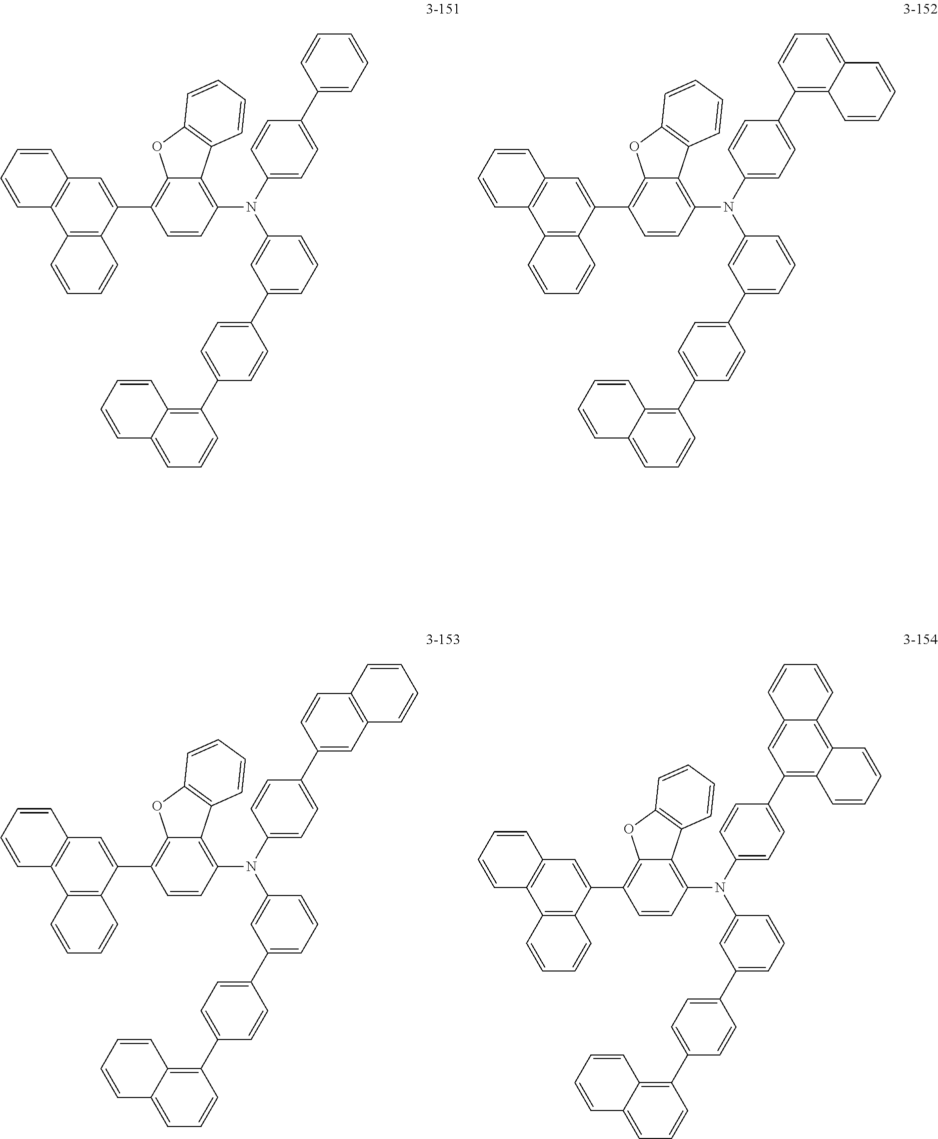 [SMILES: c1ccc(-c2ccc(N(c3cccc(-c4ccc(-c5cccc6ccccc56)cc4)c3)c3ccc(-c4cc5ccccc5c5ccccc45)c4oc5ccccc5c34)cc2)cc1 ; c1cc(-c2ccc(-c3cccc4ccccc34)cc2)cc(N(c2ccc(-c3cccc4ccccc34)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3ccc4ccccc4c3)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3cc4ccccc4c4ccccc34)cc2)c2ccc(-c3cc4ccccc4c4ccccc34)c3oc4ccccc4c23)c1]

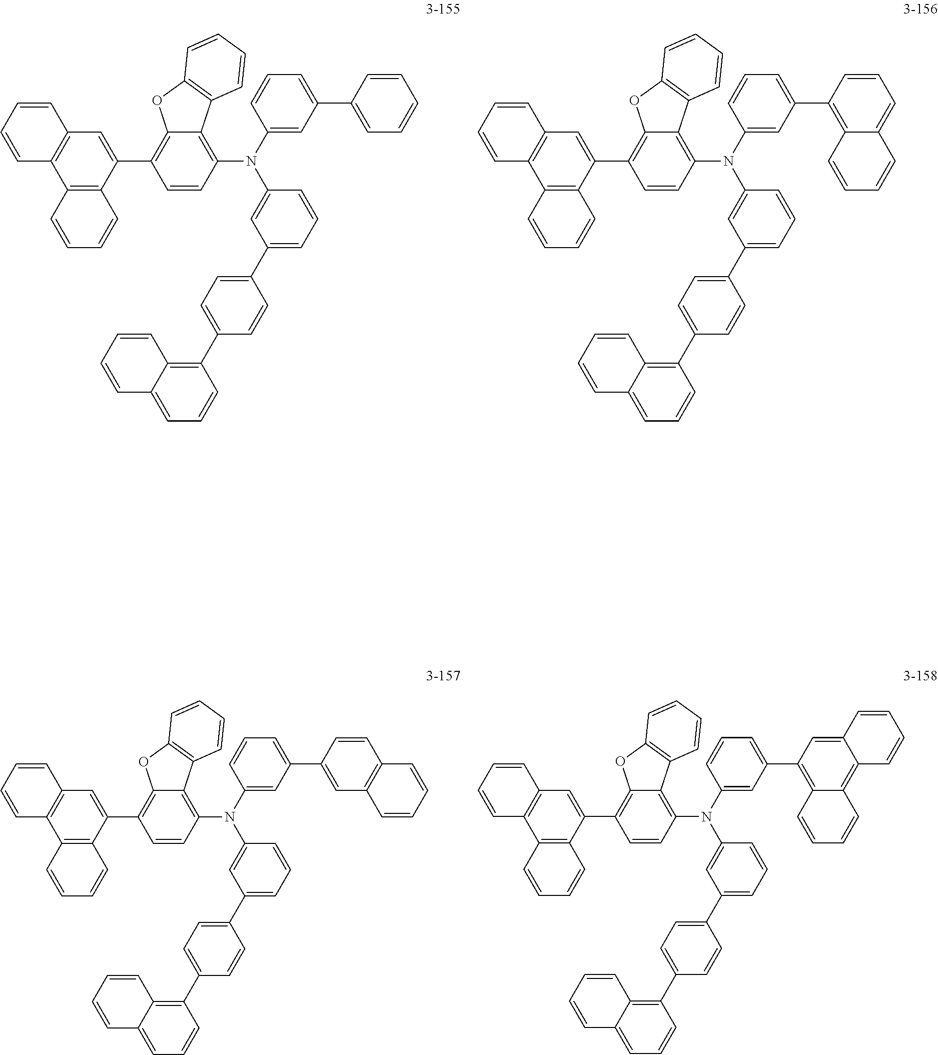 [SMILES: c1ccc(-c2cccc(N(c3cccc(-c4ccc(-c5cccc6ccccc56)cc4)c3)c3ccc(-c4cc5ccccc5c5ccccc45)c4oc5ccccc5c34)c2)cc1 ; c1cc(-c2ccc(-c3cccc4ccccc34)cc2)cc(N(c2cccc(-c3cccc4ccccc34)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3ccc4ccccc4c3)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3cc4ccccc4c4ccccc34)c2)c2ccc(-c3cc4ccccc4c4ccccc34)c3oc4ccccc4c23)c1]

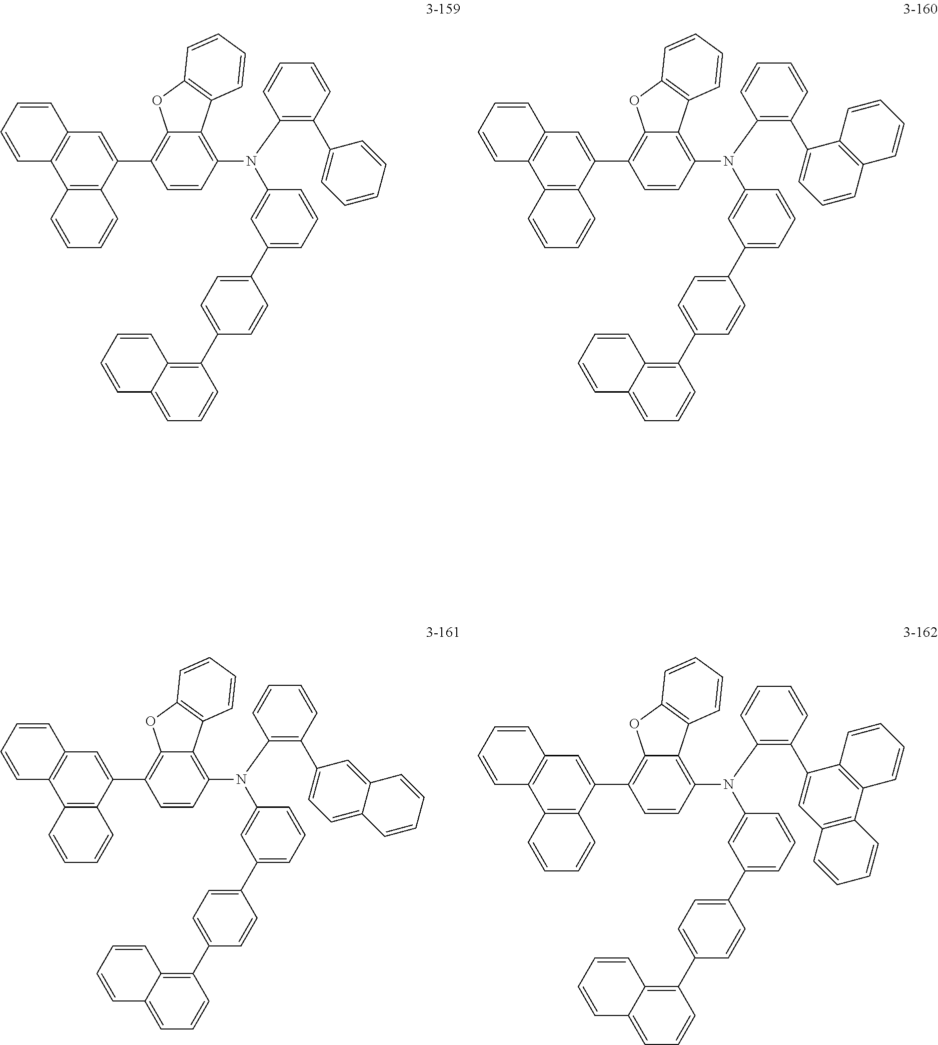 [SMILES: c1ccc(-c2ccccc2N(c2cccc(-c3ccc(-c4cccc5ccccc45)cc3)c2)c2ccc(-c3cc4ccccc4c4ccccc34)c3oc4ccccc4c23)cc1 ; c1cc(-c2ccc(-c3cccc4ccccc34)cc2)cc(N(c2ccccc2-c2cccc3ccccc23)c2ccc(-c3cc4ccccc4c4ccccc34)c3oc4ccccc4c23)c1 ; c1cc(-c2ccc(-c3cccc4ccccc34)cc2)cc(N(c2ccccc2-c2ccc3ccccc3c2)c2ccc(-c3cc4ccccc4c4ccccc34)c3oc4ccccc4c23)c1 ; c1cc(-c2ccc(-c3cccc4ccccc34)cc2)cc(N(c2ccccc2-c2cc3ccccc3c3ccccc23)c2ccc(-c3cc4ccccc4c4ccccc34)c3oc4ccccc4c23)c1]

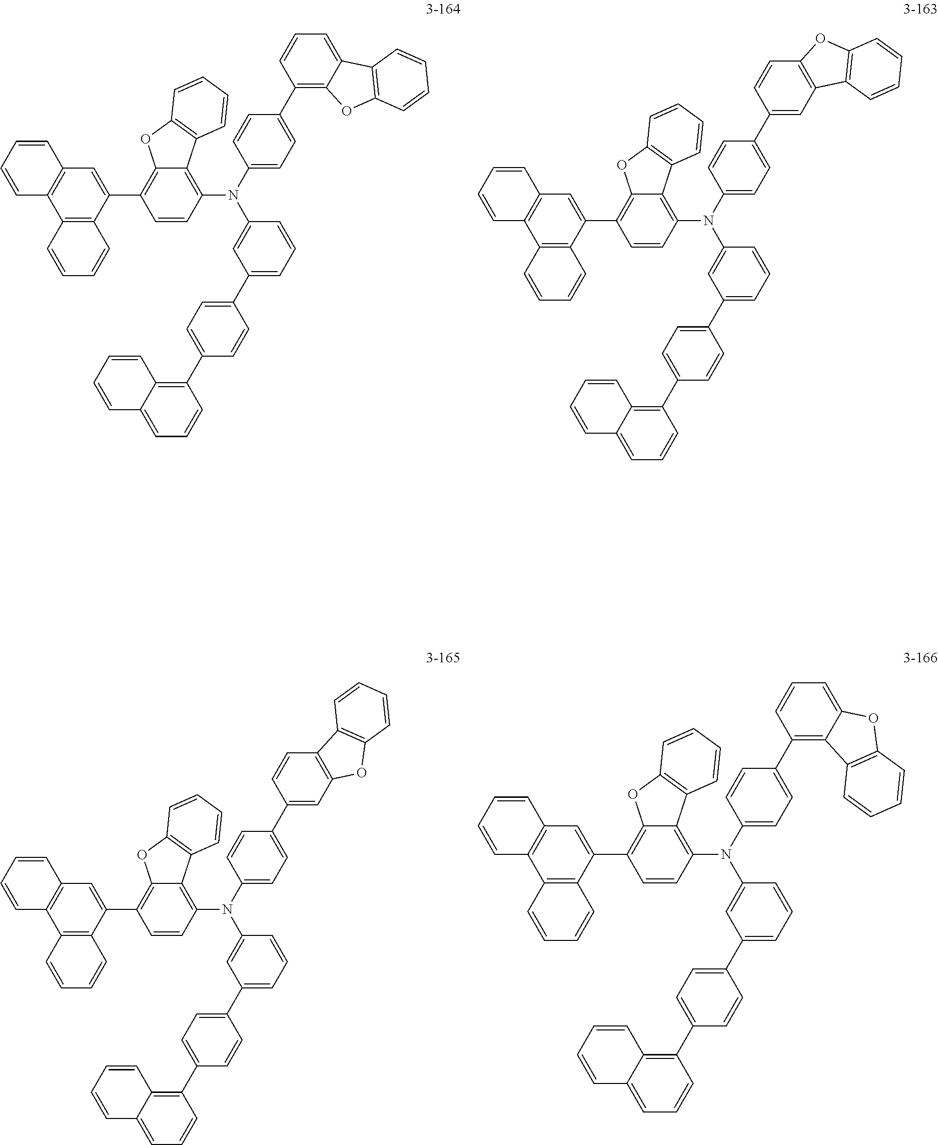 [SMILES: c1cc(-c2ccc(-c3cccc4ccccc34)cc2)cc(N(c2ccc(-c3cccc4c3oc3ccccc34)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3ccc4oc5ccccc5c4c3)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3ccc4c(c3)oc3ccccc34)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3cccc4oc5ccccc5c34)cc2)c2ccc(-c3cc4ccccc4c4ccccc34)c3oc4ccccc4c23)c1]

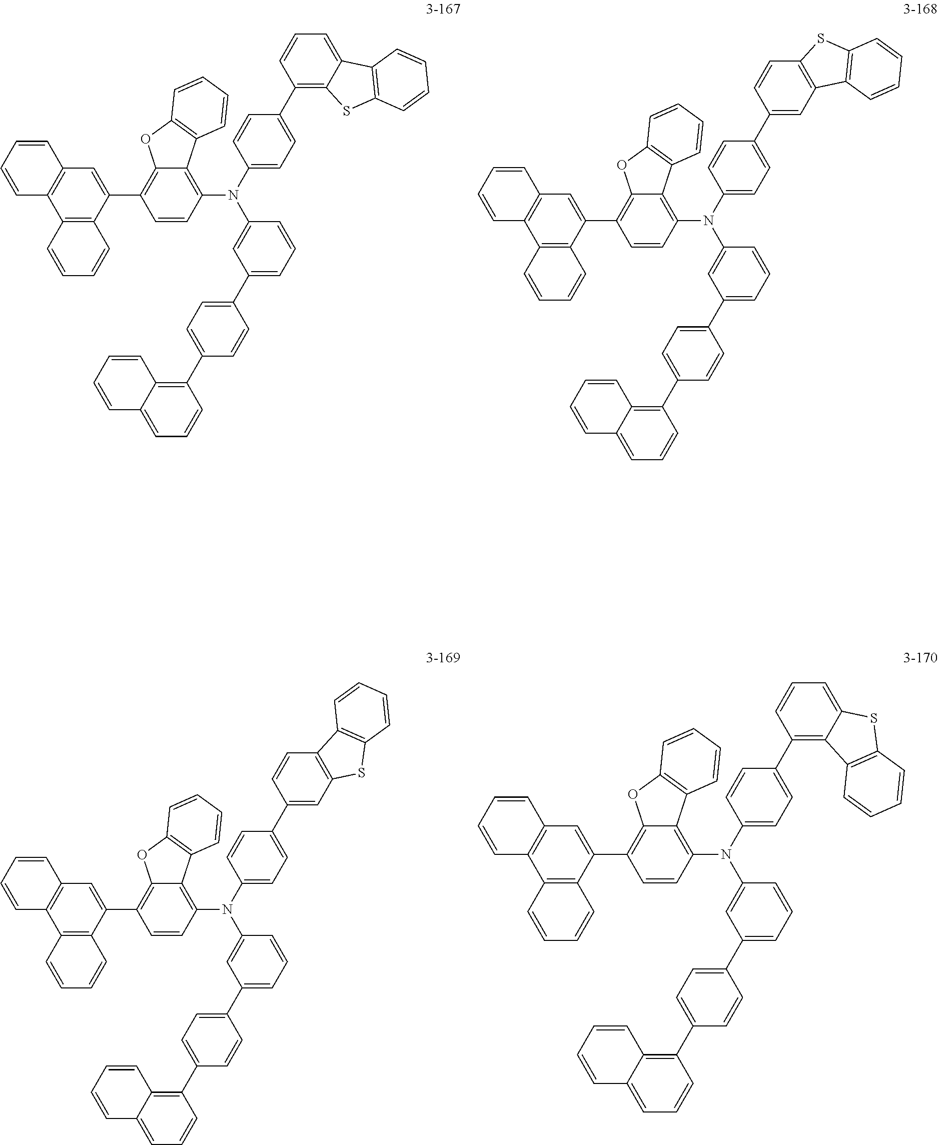 [SMILES: c1cc(-c2ccc(-c3cccc4ccccc34)cc2)cc(N(c2ccc(-c3cccc4c3sc3ccccc34)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3ccc4sc5ccccc5c4c3)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3ccc4c(c3)sc3ccccc34)cc2)c2ccc(-c3cc4ccccc4c4ccccc34)c3oc4ccccc4c23)c1 ; c1cc(-c2ccc(-c3cccc4ccccc34)cc2)cc(N(c2ccc(-c3cccc4sc5ccccc5c34)cc2)c2ccc(-c3cc4ccccc4c4ccccc34)c3oc4ccccc4c23)c1]

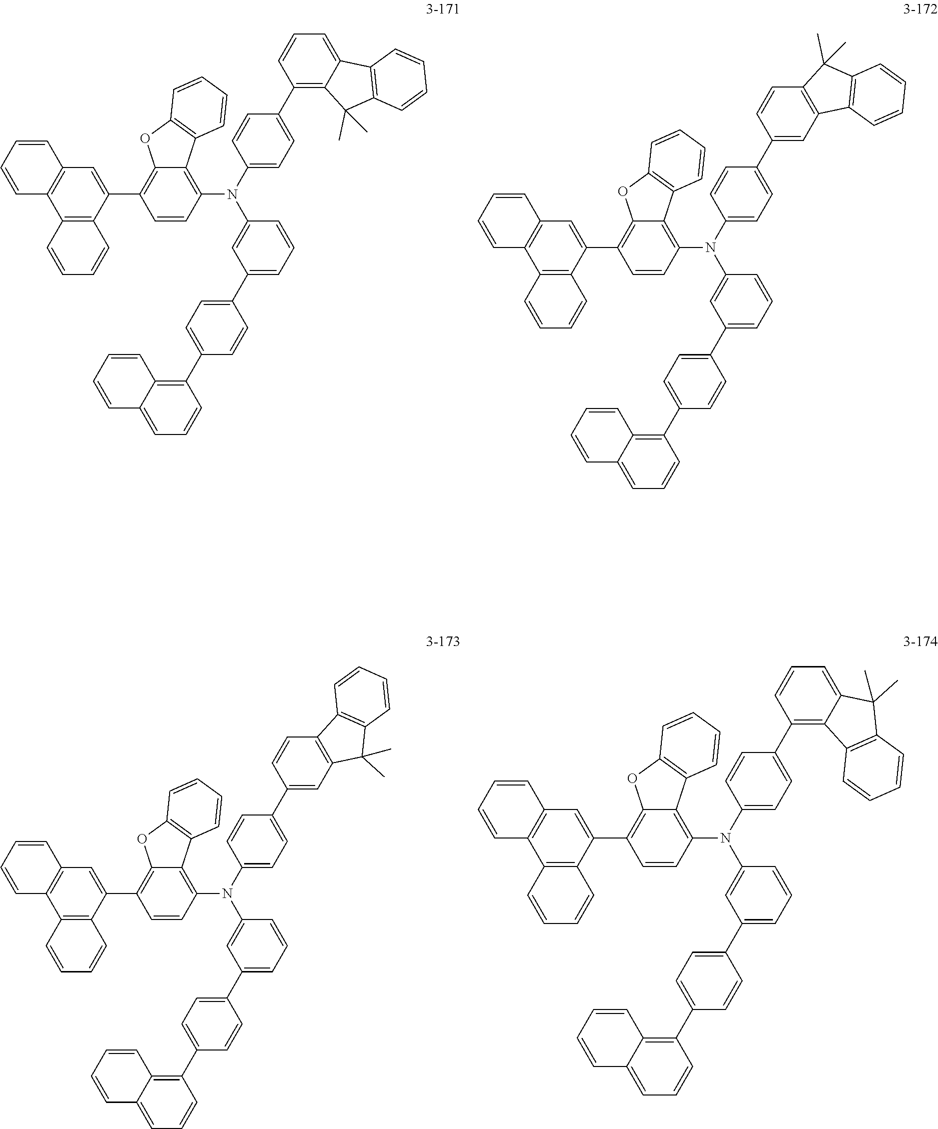 [SMILES: CC1(C)c2ccccc2-c2cccc(-c3ccc(N(c4cccc(-c5ccc(-c6cccc7ccccc67)cc5)c4)c4ccc(-c5cc6ccccc6c6ccccc56)c5oc6ccccc6c45)cc3)c21 ; CC1(C)c2ccccc2-c2cc(-c3ccc(N(c4cccc(-c5ccc(-c6cccc7ccccc67)cc5)c4)c4ccc(-c5cc6ccccc6c6ccccc56)c5oc6ccccc6c45)cc3)ccc21 ; CC1(C)c2ccccc2-c2ccc(-c3ccc(N(c4cccc(-c5ccc(-c6cccc7ccccc67)cc5)c4)c4ccc(-c5cc6ccccc6c6ccccc56)c5oc6ccccc6c45)cc3)cc21 ; CC1(C)c2ccccc2-c2c(-c3ccc(N(c4cccc(-c5ccc(-c6cccc7ccccc67)cc5)c4)c4ccc(-c5cc6ccccc6c6ccccc56)c5oc6ccccc6c45)cc3)cccc21]

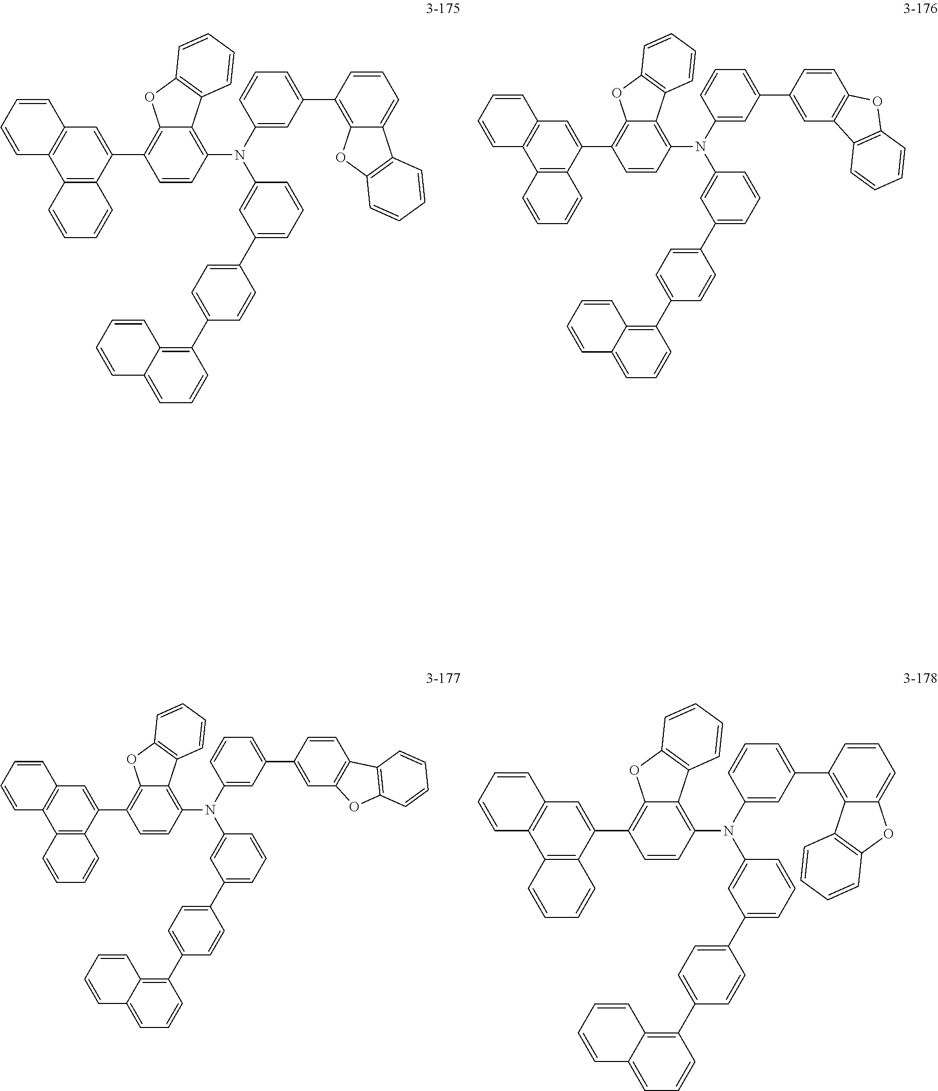 [SMILES: c1cc(-c2ccc(-c3cccc4ccccc34)cc2)cc(N(c2cccc(-c3cccc4c3oc3ccccc34)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3ccc4oc5ccccc5c4c3)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3ccc4c(c3)oc3ccccc34)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3cccc4oc5ccccc5c34)c2)c2ccc(-c3cc4ccccc4c4ccccc34)c3oc4ccccc4c23)c1]

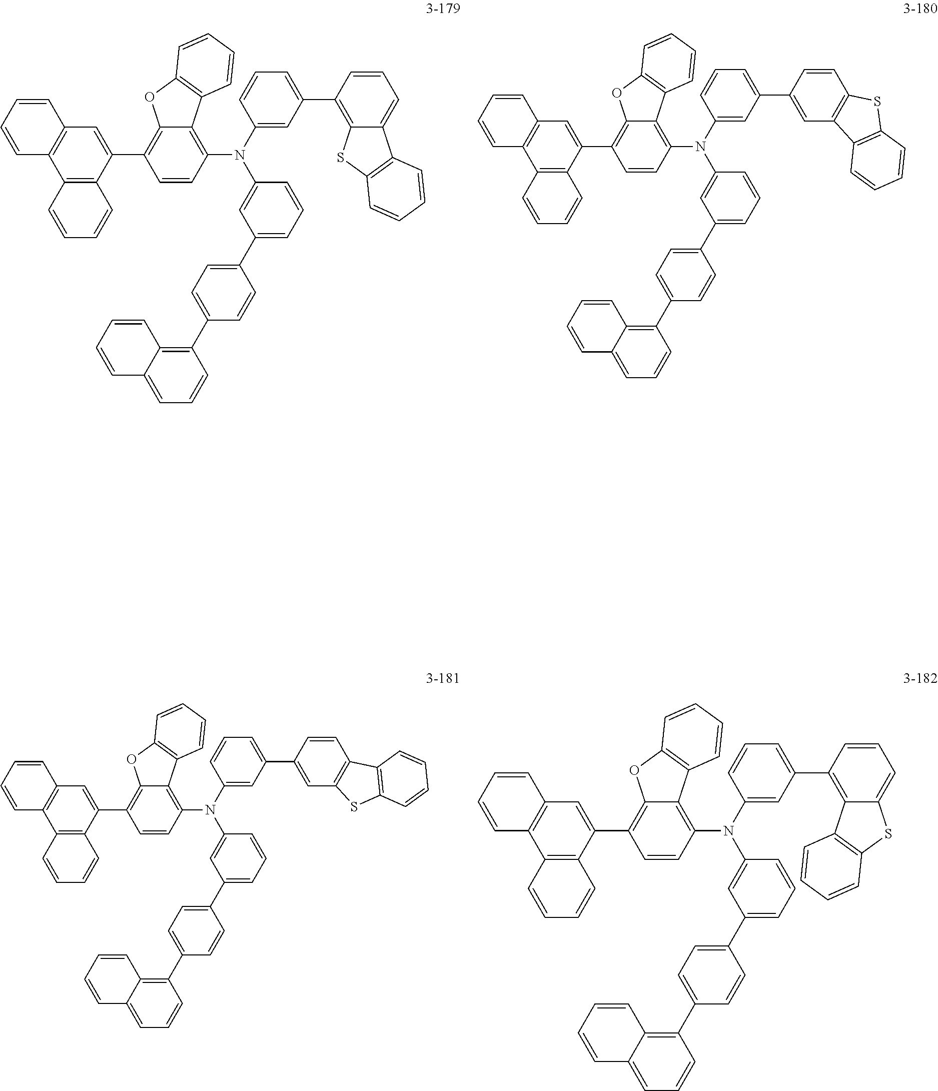 [SMILES: c1cc(-c2ccc(-c3cccc4ccccc34)cc2)cc(N(c2cccc(-c3cccc4c3sc3ccccc34)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3ccc4sc5ccccc5c4c3)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3ccc4c(c3)sc3ccccc34)c2)c2ccc(-c3cc4ccccc4c4ccccc34)c3oc4ccccc4c23)c1 ; c1cc(-c2ccc(-c3cccc4ccccc34)cc2)cc(N(c2cccc(-c3cccc4sc5ccccc5c34)c2)c2ccc(-c3cc4ccccc4c4ccccc34)c3oc4ccccc4c23)c1]

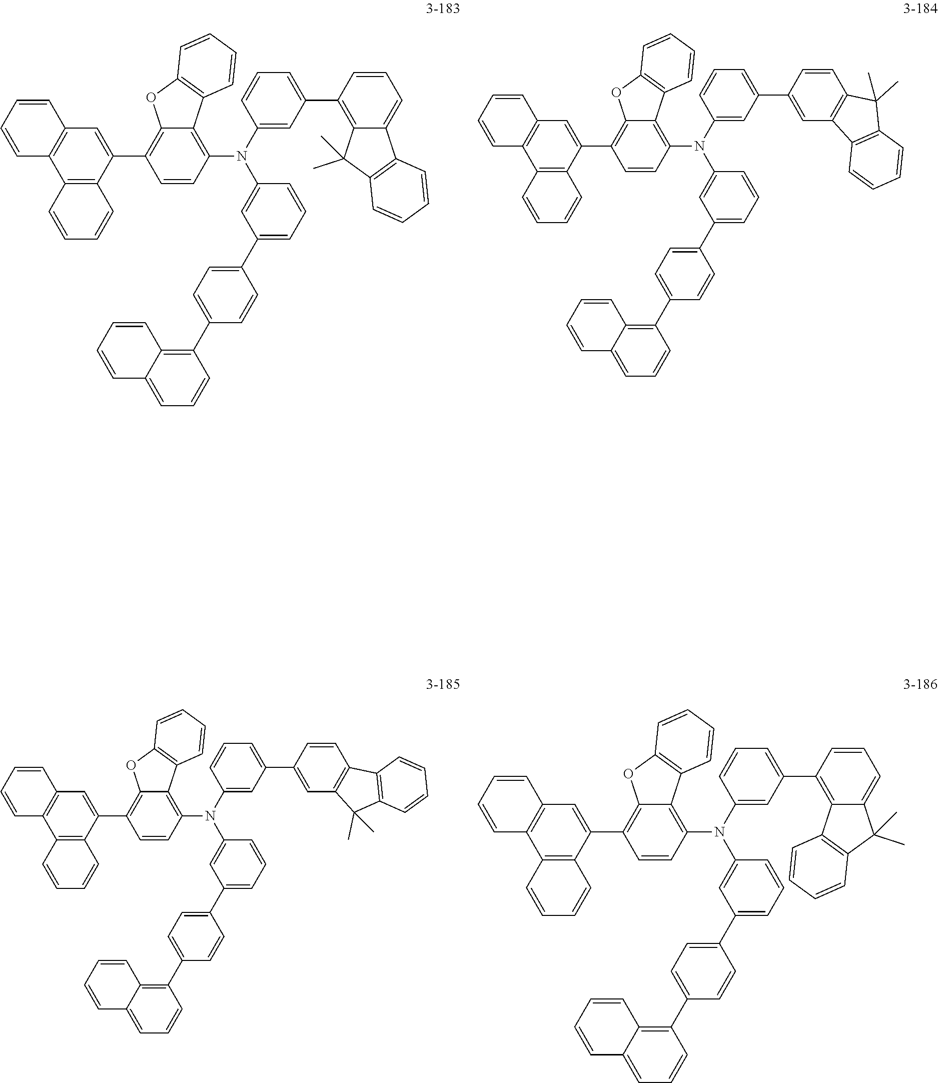 [SMILES: CC1(C)c2ccccc2-c2cccc(-c3cccc(N(c4cccc(-c5ccc(-c6cccc7ccccc67)cc5)c4)c4ccc(-c5cc6ccccc6c6ccccc56)c5oc6ccccc6c45)c3)c21 ; CC1(C)c2ccccc2-c2cc(-c3cccc(N(c4cccc(-c5ccc(-c6cccc7ccccc67)cc5)c4)c4ccc(-c5cc6ccccc6c6ccccc56)c5oc6ccccc6c45)c3)ccc21 ; CC1(C)c2ccccc2-c2ccc(-c3cccc(N(c4cccc(-c5ccc(-c6cccc7ccccc67)cc5)c4)c4ccc(-c5cc6ccccc6c6ccccc56)c5oc6ccccc6c45)c3)cc21 ; CC1(C)c2ccccc2-c2c(-c3cccc(N(c4cccc(-c5ccc(-c6cccc7ccccc67)cc5)c4)c4ccc(-c5cc6ccccc6c6ccccc56)c5oc6ccccc6c45)c3)cccc21]

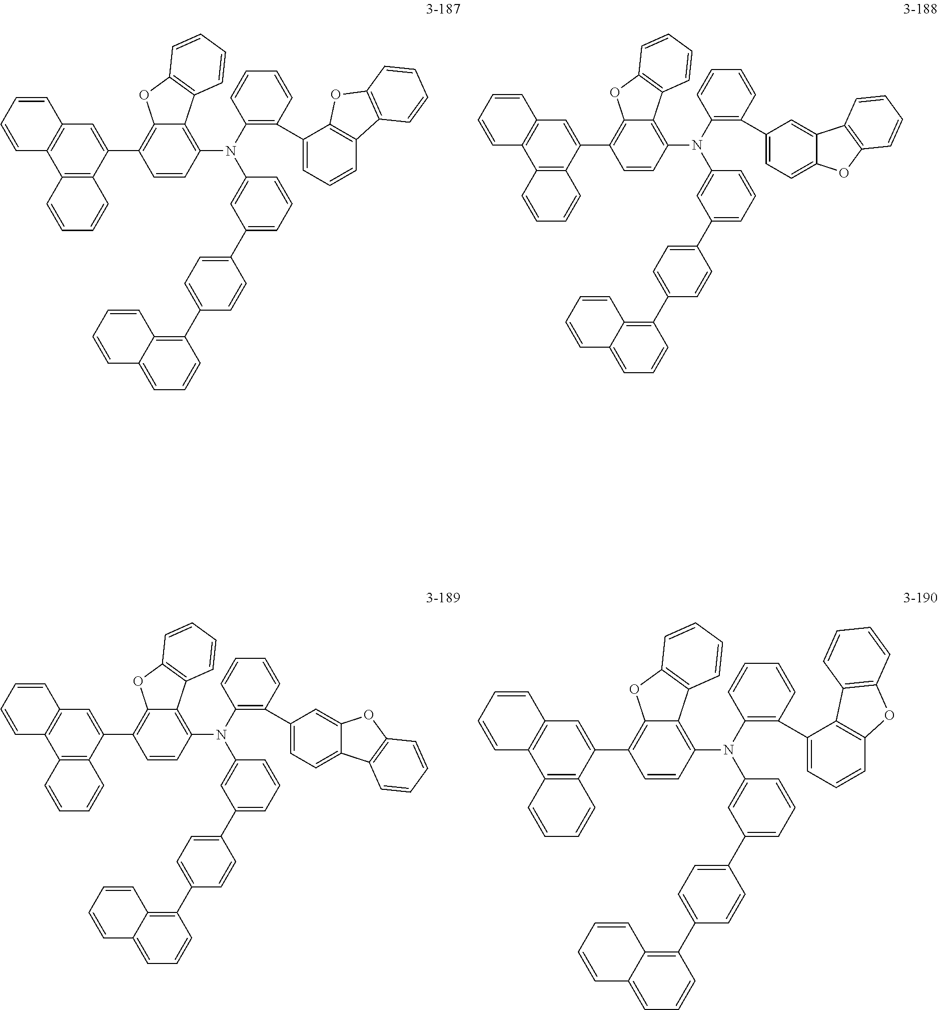 [SMILES: c1cc(-c2ccc(-c3cccc4ccccc34)cc2)cc(N(c2ccccc2-c2cccc3c2oc2ccccc23)c2ccc(-c3cc4ccccc4c4ccccc34)c3oc4ccccc4c23)c1 ; c1cc(-c2ccc(-c3cccc4ccccc34)cc2)cc(N(c2ccccc2-c2ccc3oc4ccccc4c3c2)c2ccc(-c3cc4ccccc4c4ccccc34)c3oc4ccccc4c23)c1 ; c1cc(-c2ccc(-c3cccc4ccccc34)cc2)cc(N(c2ccccc2-c2ccc3c(c2)oc2ccccc23)c2ccc(-c3cc4ccccc4c4ccccc34)c3oc4ccccc4c23)c1 ; c1cc(-c2ccc(-c3cccc4ccccc34)cc2)cc(N(c2ccccc2-c2cccc3oc4ccccc4c23)c2ccc(-c3cc4ccccc4c4ccccc34)c3oc4ccccc4c23)c1]

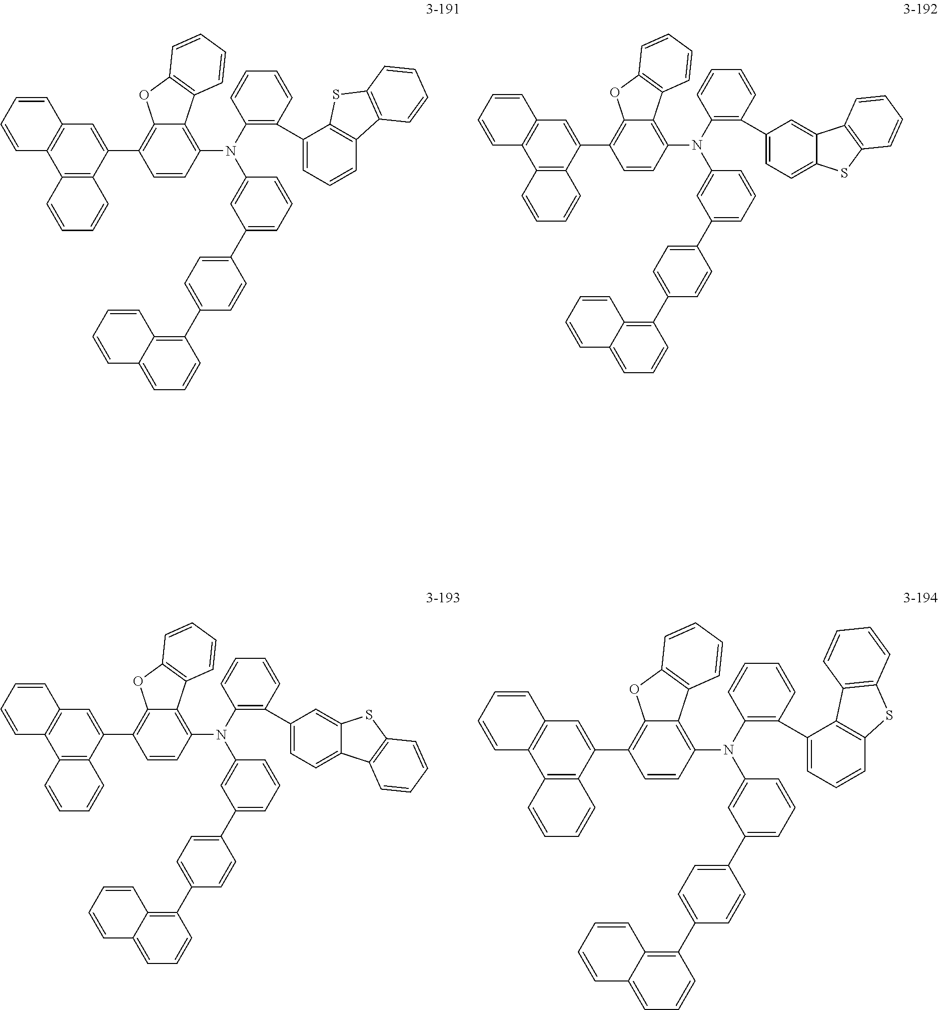 [SMILES: c1cc(-c2ccc(-c3cccc4ccccc34)cc2)cc(N(c2ccccc2-c2cccc3c2sc2ccccc23)c2ccc(-c3cc4ccccc4c4ccccc34)c3oc4ccccc4c23)c1 ; c1cc(-c2ccc(-c3cccc4ccccc34)cc2)cc(N(c2ccccc2-c2ccc3sc4ccccc4c3c2)c2ccc(-c3cc4ccccc4c4ccccc34)c3oc4ccccc4c23)c1 ; c1cc(-c2ccc(-c3cccc4ccccc34)cc2)cc(N(c2ccccc2-c2ccc3c(c2)sc2ccccc23)c2ccc(-c3cc4ccccc4c4ccccc34)c3oc4ccccc4c23)c1 ; c1cc(-c2ccc(-c3cccc4ccccc34)cc2)cc(N(c2ccccc2-c2cccc3sc4ccccc4c23)c2ccc(-c3cc4ccccc4c4ccccc34)c3oc4ccccc4c23)c1]

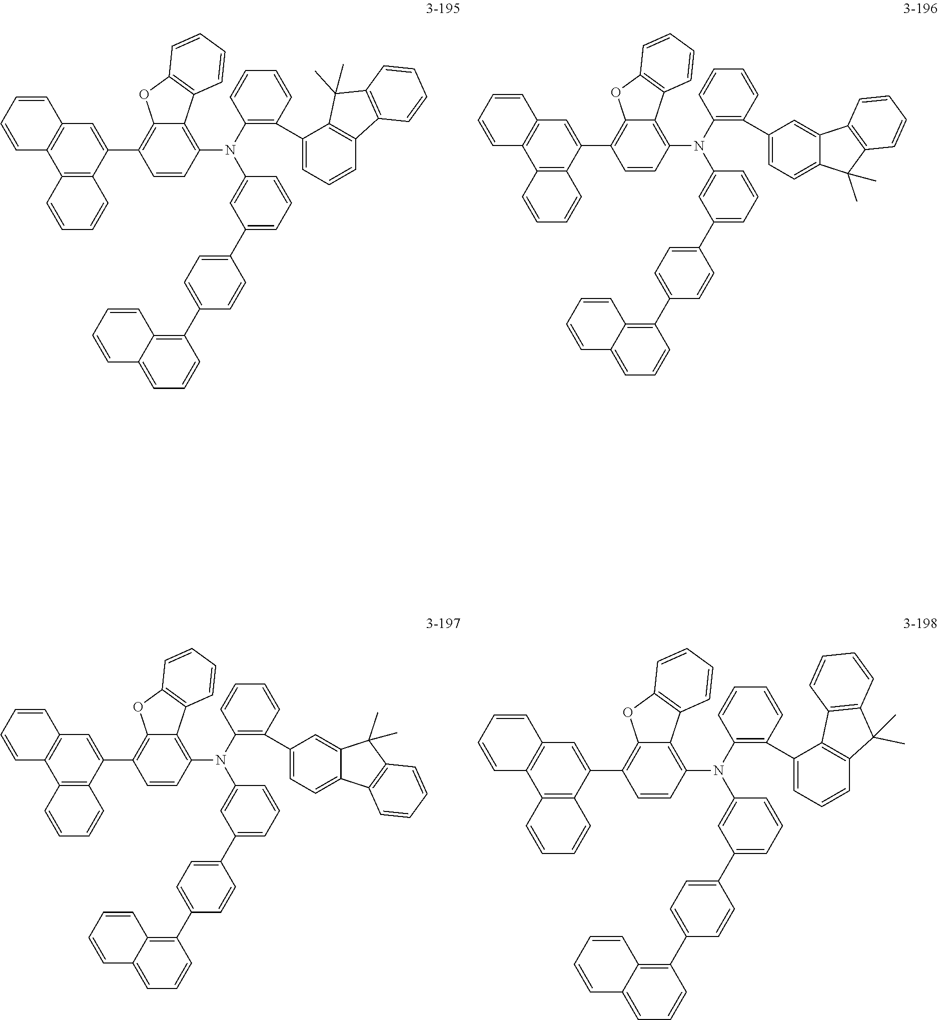 [SMILES: CC1(C)c2ccccc2-c2cccc(-c3ccccc3N(c3cccc(-c4ccc(-c5cccc6ccccc56)cc4)c3)c3ccc(-c4cc5ccccc5c5ccccc45)c4oc5ccccc5c34)c21 ; CC1(C)c2ccccc2-c2cc(-c3ccccc3N(c3cccc(-c4ccc(-c5cccc6ccccc56)cc4)c3)c3ccc(-c4cc5ccccc5c5ccccc45)c4oc5ccccc5c34)ccc21 ; CC1(C)c2ccccc2-c2ccc(-c3ccccc3N(c3cccc(-c4ccc(-c5cccc6ccccc56)cc4)c3)c3ccc(-c4cc5ccccc5c5ccccc45)c4oc5ccccc5c34)cc21 ; CC1(C)c2ccccc2-c2c(-c3ccccc3N(c3cccc(-c4ccc(-c5cccc6ccccc56)cc4)c3)c3ccc(-c4cc5ccccc5c5ccccc45)c4oc5ccccc5c34)cccc21]

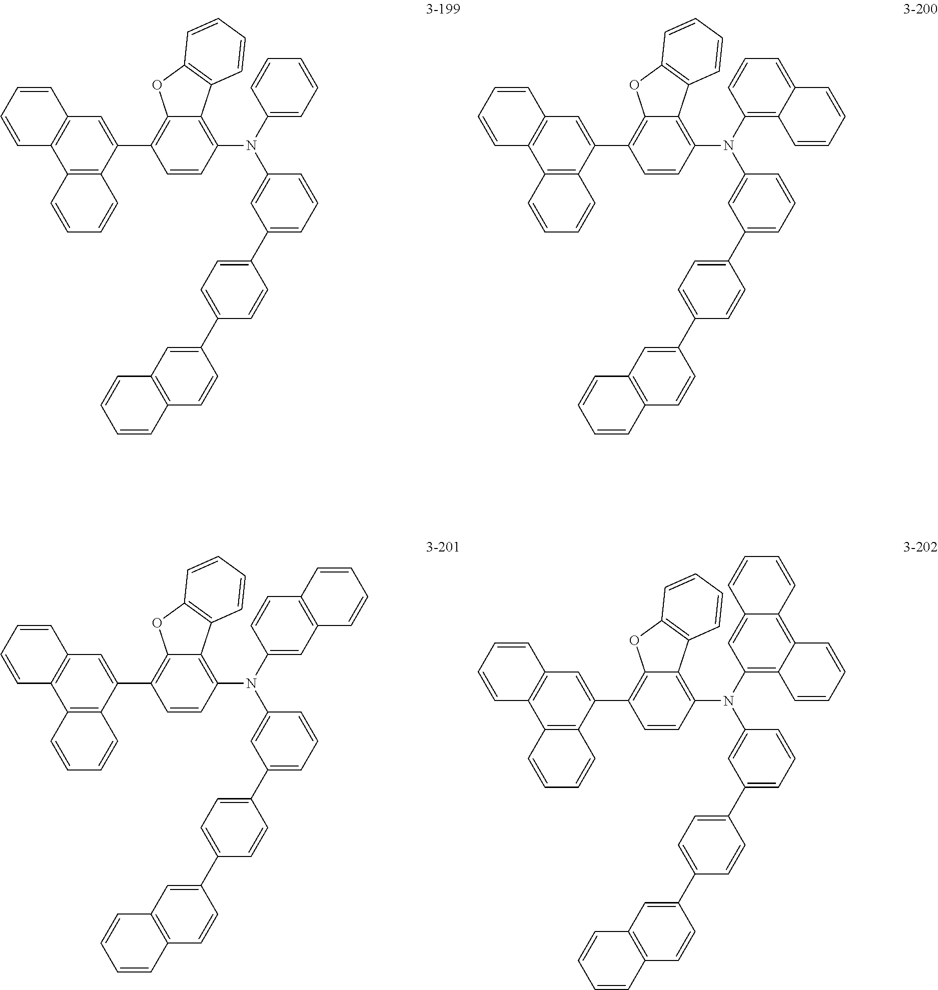 [SMILES: c1ccc(N(c2cccc(-c3ccc(-c4ccc5ccccc5c4)cc3)c2)c2ccc(-c3cc4ccccc4c4ccccc34)c3oc4ccccc4c23)cc1 ; c1cc(-c2ccc(-c3ccc4ccccc4c3)cc2)cc(N(c2cccc3ccccc23)c2ccc(-c3cc4ccccc4c4ccccc34)c3oc4ccccc4c23)c1 ; c1cc(-c2ccc(-c3ccc4ccccc4c3)cc2)cc(N(c2ccc3ccccc3c2)c2ccc(-c3cc4ccccc4c4ccccc34)c3oc4ccccc4c23)c1 ; c1cc(-c2ccc(-c3ccc4ccccc4c3)cc2)cc(N(c2cc3ccccc3c3ccccc23)c2ccc(-c3cc4ccccc4c4ccccc34)c3oc4ccccc4c23)c1]

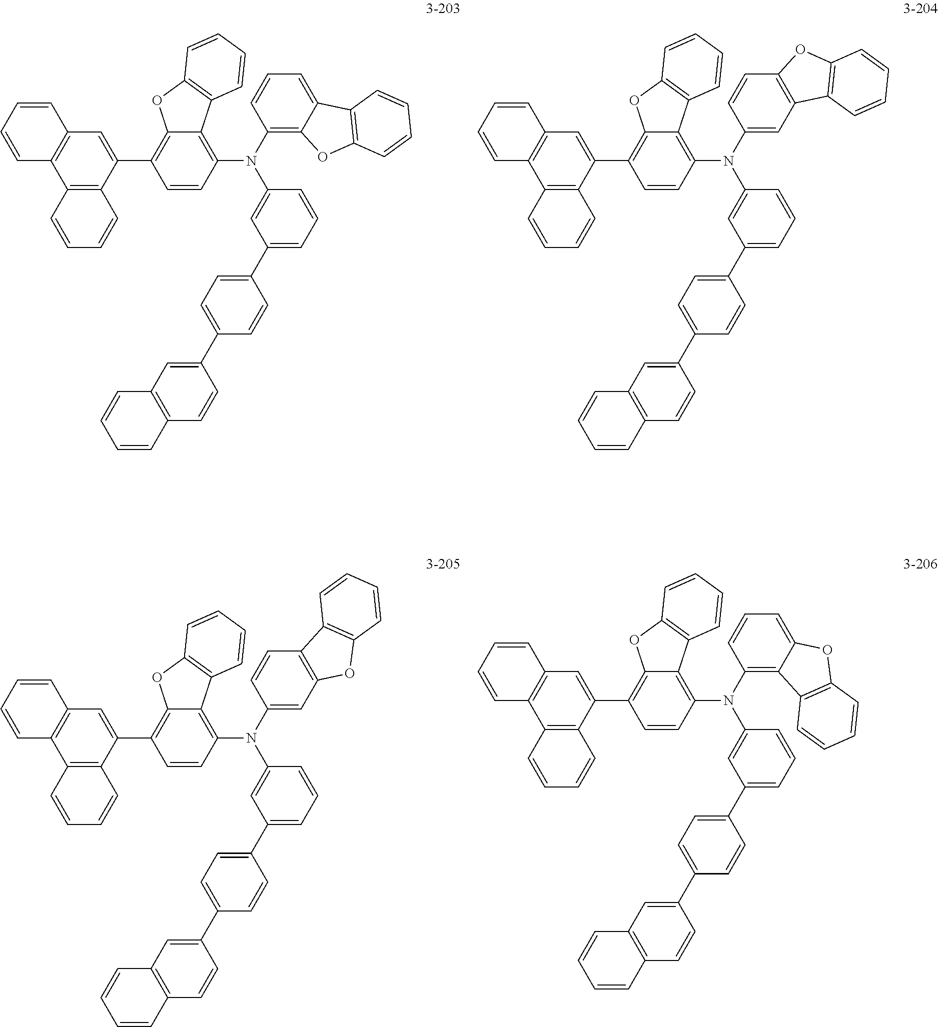 [SMILES: c1cc(-c2ccc(-c3ccc4ccccc4c3)cc2)cc(N(c2cccc3c2oc2ccccc23)c2ccc(-c3cc4ccccc4c4ccccc34)c3oc4ccccc4c23)c1 ; c1cc(-c2ccc(-c3ccc4ccccc4c3)cc2)cc(N(c2ccc3oc4ccccc4c3c2)c2ccc(-c3cc4ccccc4c4ccccc34)c3oc4ccccc4c23)c1 ; c1cc(-c2ccc(-c3ccc4ccccc4c3)cc2)cc(N(c2ccc3c(c2)oc2ccccc23)c2ccc(-c3cc4ccccc4c4ccccc34)c3oc4ccccc4c23)c1 ; c1cc(-c2ccc(-c3ccc4ccccc4c3)cc2)cc(N(c2cccc3oc4ccccc4c23)c2ccc(-c3cc4ccccc4c4ccccc34)c3oc4ccccc4c23)c1]

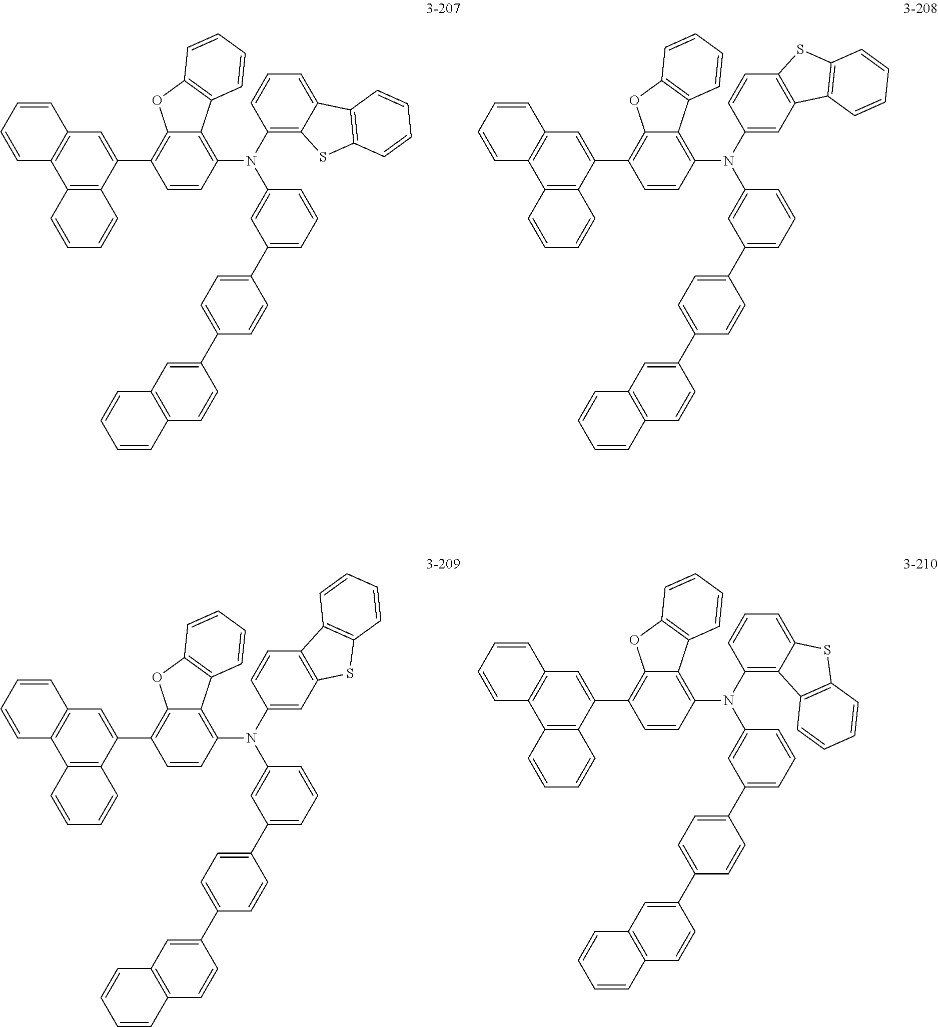 [SMILES: c1cc(-c2ccc(-c3ccc4ccccc4c3)cc2)cc(N(c2cccc3c2sc2ccccc23)c2ccc(-c3cc4ccccc4c4ccccc34)c3oc4ccccc4c23)c1 ; c1cc(-c2ccc(-c3ccc4ccccc4c3)cc2)cc(N(c2ccc3sc4ccccc4c3c2)c2ccc(-c3cc4ccccc4c4ccccc34)c3oc4ccccc4c23)c1 ; c1cc(-c2ccc(-c3ccc4ccccc4c3)cc2)cc(N(c2ccc3c(c2)sc2ccccc23)c2ccc(-c3cc4ccccc4c4ccccc34)c3oc4ccccc4c23)c1 ; c1cc(-c2ccc(-c3ccc4ccccc4c3)cc2)cc(N(c2cccc3sc4ccccc4c23)c2ccc(-c3cc4ccccc4c4ccccc34)c3oc4ccccc4c23)c1]

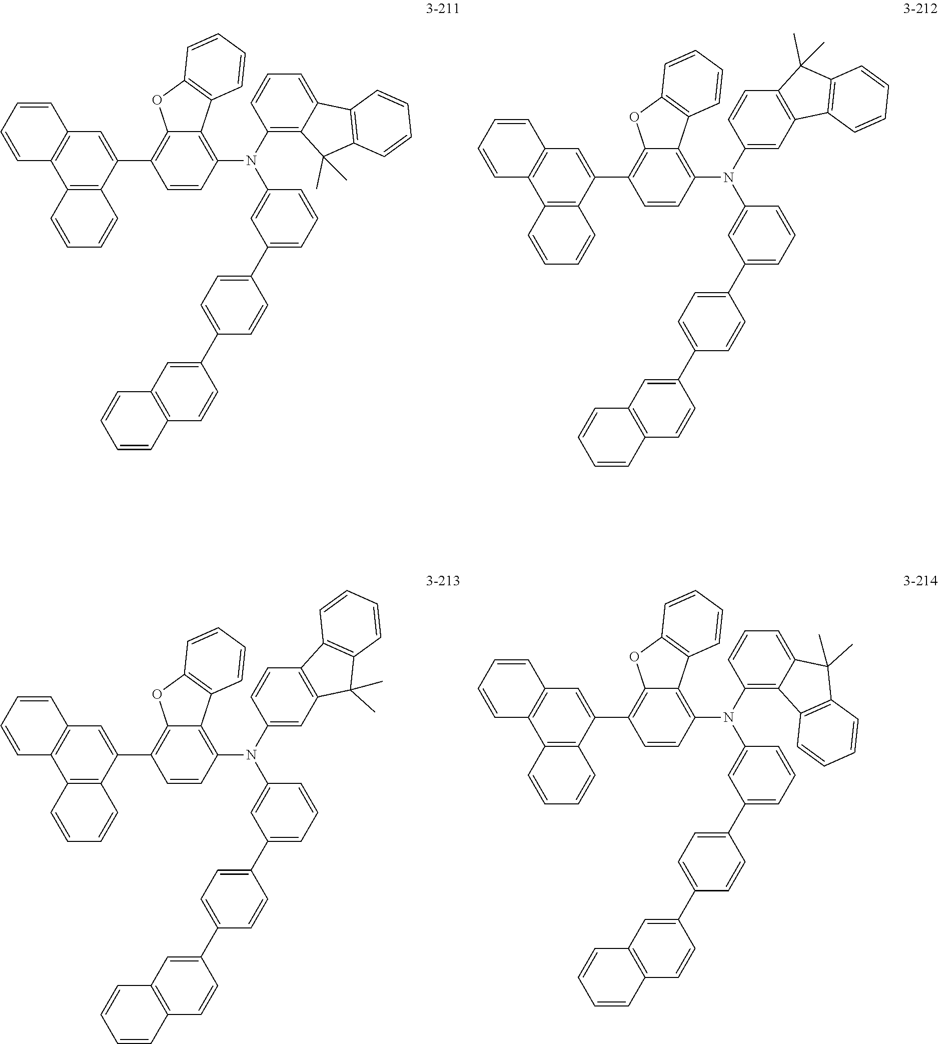 [SMILES: CC1(C)c2ccccc2-c2cccc(N(c3cccc(-c4ccc(-c5ccc6ccccc6c5)cc4)c3)c3ccc(-c4cc5ccccc5c5ccccc45)c4oc5ccccc5c34)c21 ; CC1(C)c2ccccc2-c2cc(N(c3cccc(-c4ccc(-c5ccc6ccccc6c5)cc4)c3)c3ccc(-c4cc5ccccc5c5ccccc45)c4oc5ccccc5c34)ccc21 ; CC1(C)c2ccccc2-c2ccc(N(c3cccc(-c4ccc(-c5ccc6ccccc6c5)cc4)c3)c3ccc(-c4cc5ccccc5c5ccccc45)c4oc5ccccc5c34)cc21 ; CC1(C)c2ccccc2-c2c(N(c3cccc(-c4ccc(-c5ccc6ccccc6c5)cc4)c3)c3ccc(-c4cc5ccccc5c5ccccc45)c4oc5ccccc5c34)cccc21]

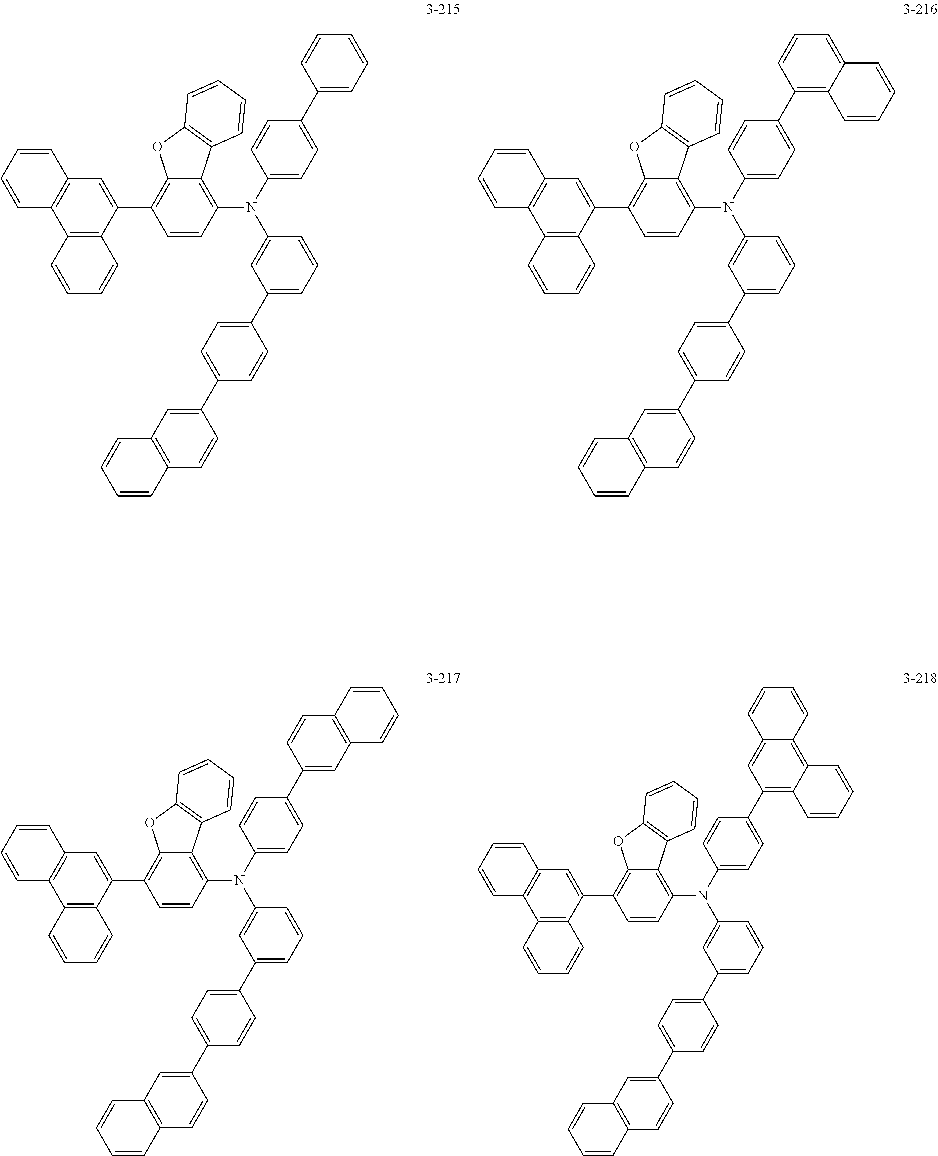 [SMILES: c1ccc(-c2ccc(N(c3cccc(-c4ccc(-c5ccc6ccccc6c5)cc4)c3)c3ccc(-c4cc5ccccc5c5ccccc45)c4oc5ccccc5c34)cc2)cc1 ; c1cc(-c2ccc(-c3ccc4ccccc4c3)cc2)cc(N(c2ccc(-c3cccc4ccccc34)cc2)c2ccc(-c3cc4ccccc4c4ccccc34)c3oc4ccccc4c23)c1 ; c1cc(-c2ccc(-c3ccc4ccccc4c3)cc2)cc(N(c2ccc(-c3ccc4ccccc4c3)cc2)c2ccc(-c3cc4ccccc4c4ccccc34)c3oc4ccccc4c23)c1 ; c1cc(-c2ccc(-c3ccc4ccccc4c3)cc2)cc(N(c2ccc(-c3cc4ccccc4c4ccccc34)cc2)c2ccc(-c3cc4ccccc4c4ccccc34)c3oc4ccccc4c23)c1]

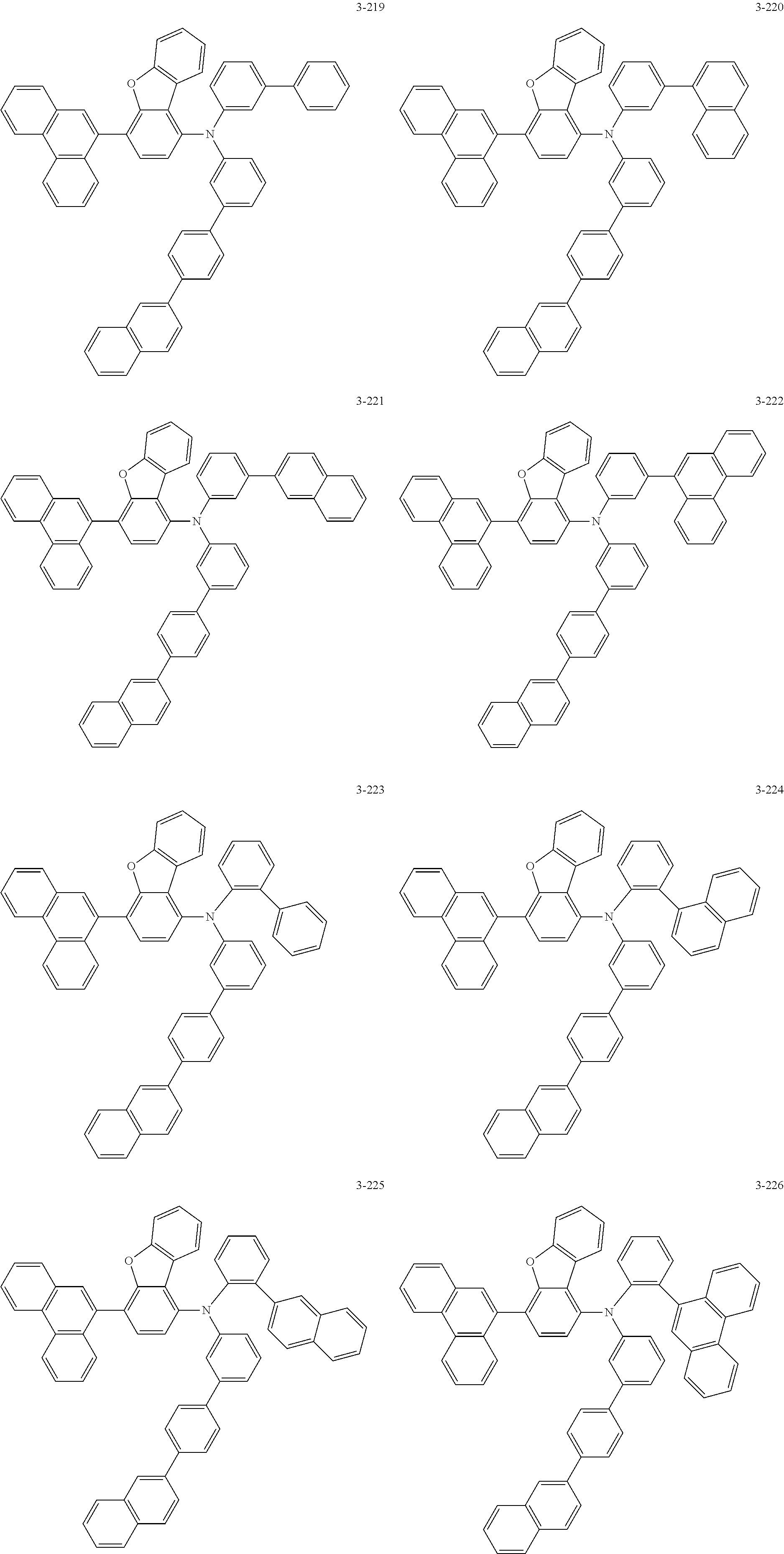

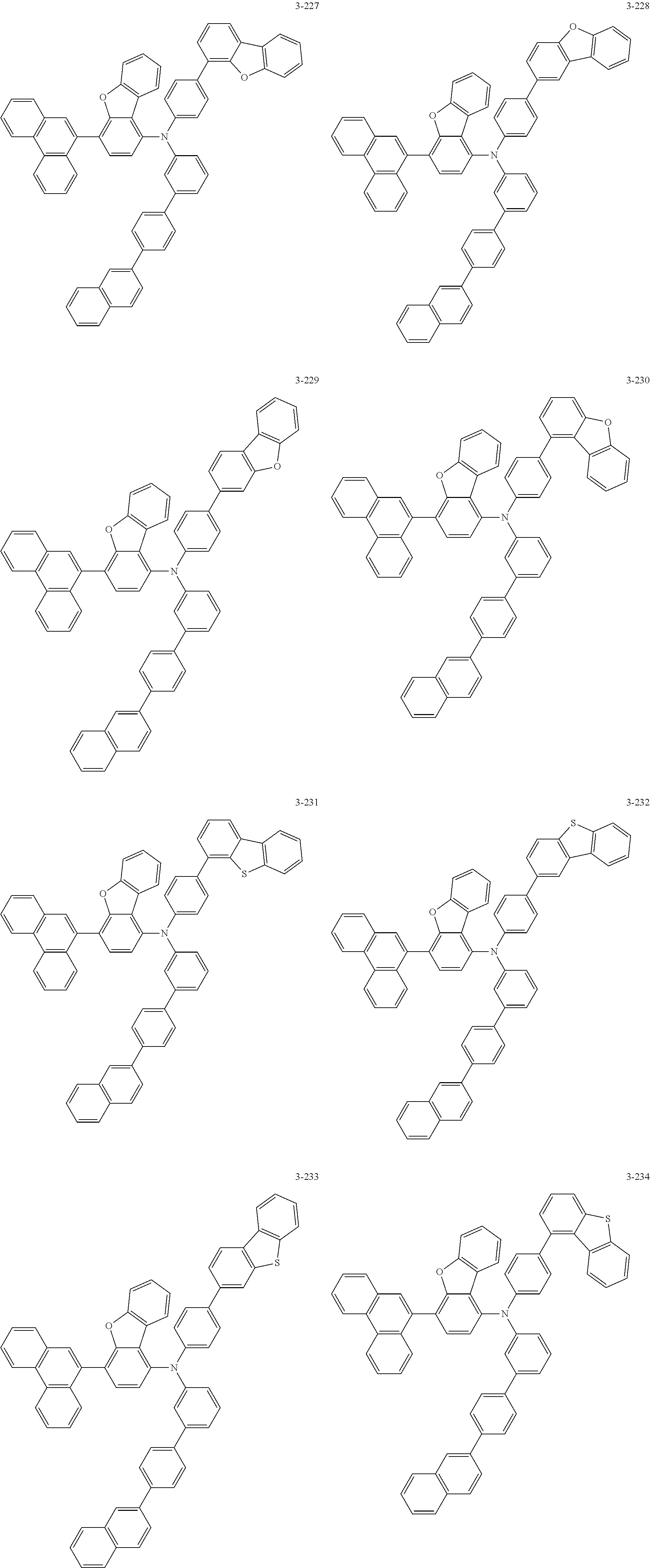

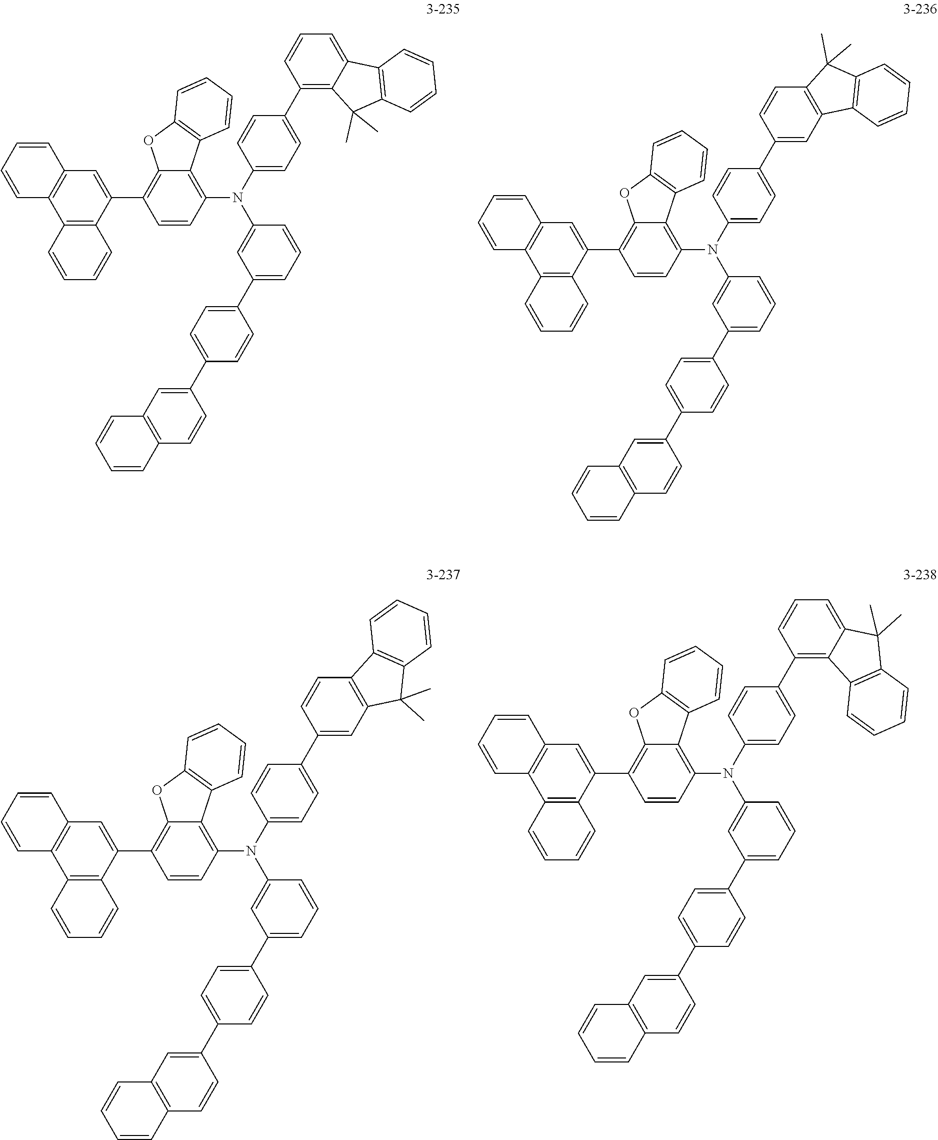 [SMILES: CC1(C)c2ccccc2-c2cccc(-c3ccc(N(c4cccc(-c5ccc(-c6ccc7ccccc7c6)cc5)c4)c4ccc(-c5cc6ccccc6c6ccccc56)c5oc6ccccc6c45)cc3)c21 ; CC1(C)c2ccccc2-c2cc(-c3ccc(N(c4cccc(-c5ccc(-c6ccc7ccccc7c6)cc5)c4)c4ccc(-c5cc6ccccc6c6ccccc56)c5oc6ccccc6c45)cc3)ccc21 ; CC1(C)c2ccccc2-c2ccc(-c3ccc(N(c4cccc(-c5ccc(-c6ccc7ccccc7c6)cc5)c4)c4ccc(-c5cc6ccccc6c6ccccc56)c5oc6ccccc6c45)cc3)cc21 ; CC1(C)c2ccccc2-c2c(-c3ccc(N(c4cccc(-c5ccc(-c6ccc7ccccc7c6)cc5)c4)c4ccc(-c5cc6ccccc6c6ccccc56)c5oc6ccccc6c45)cc3)cccc21]

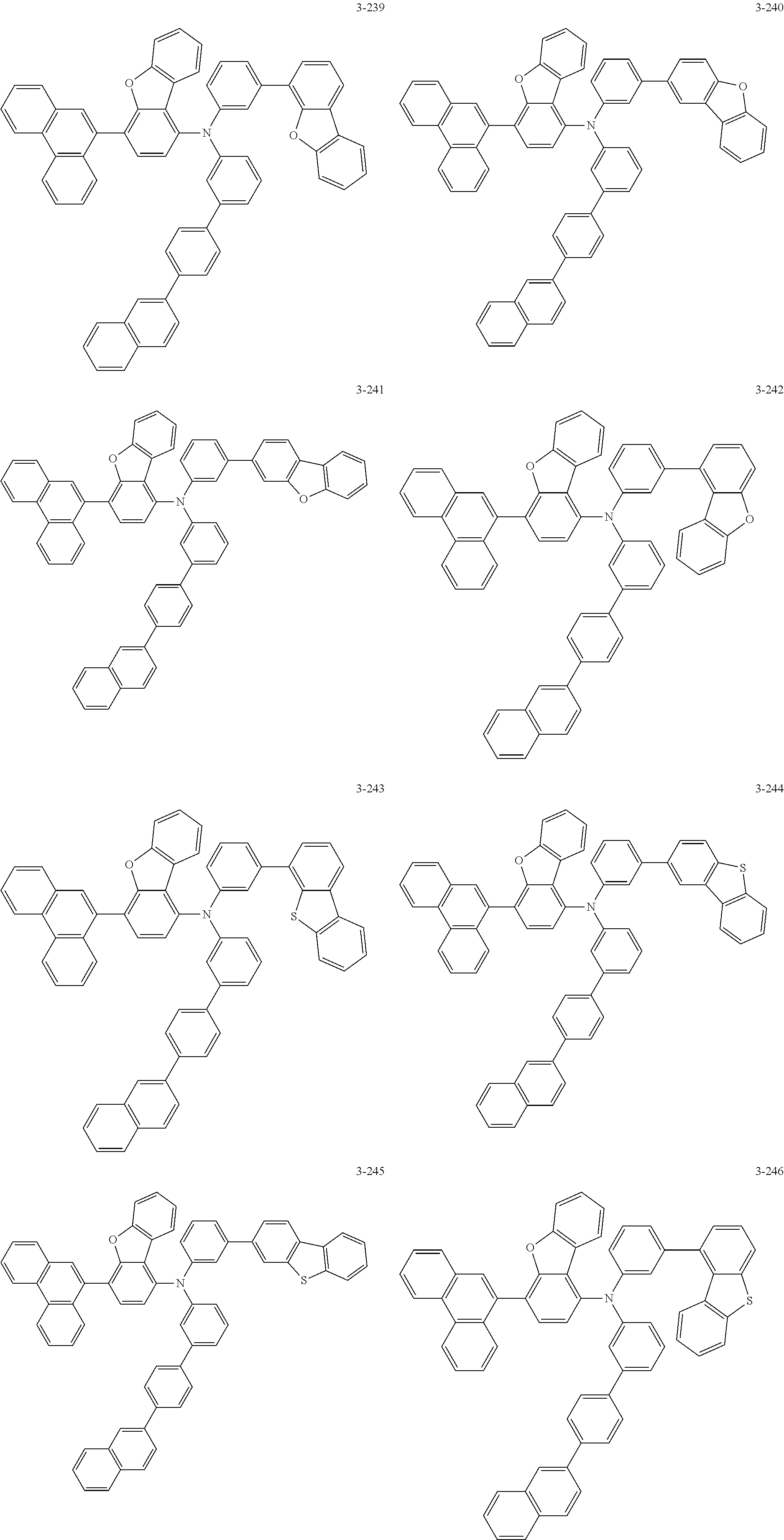

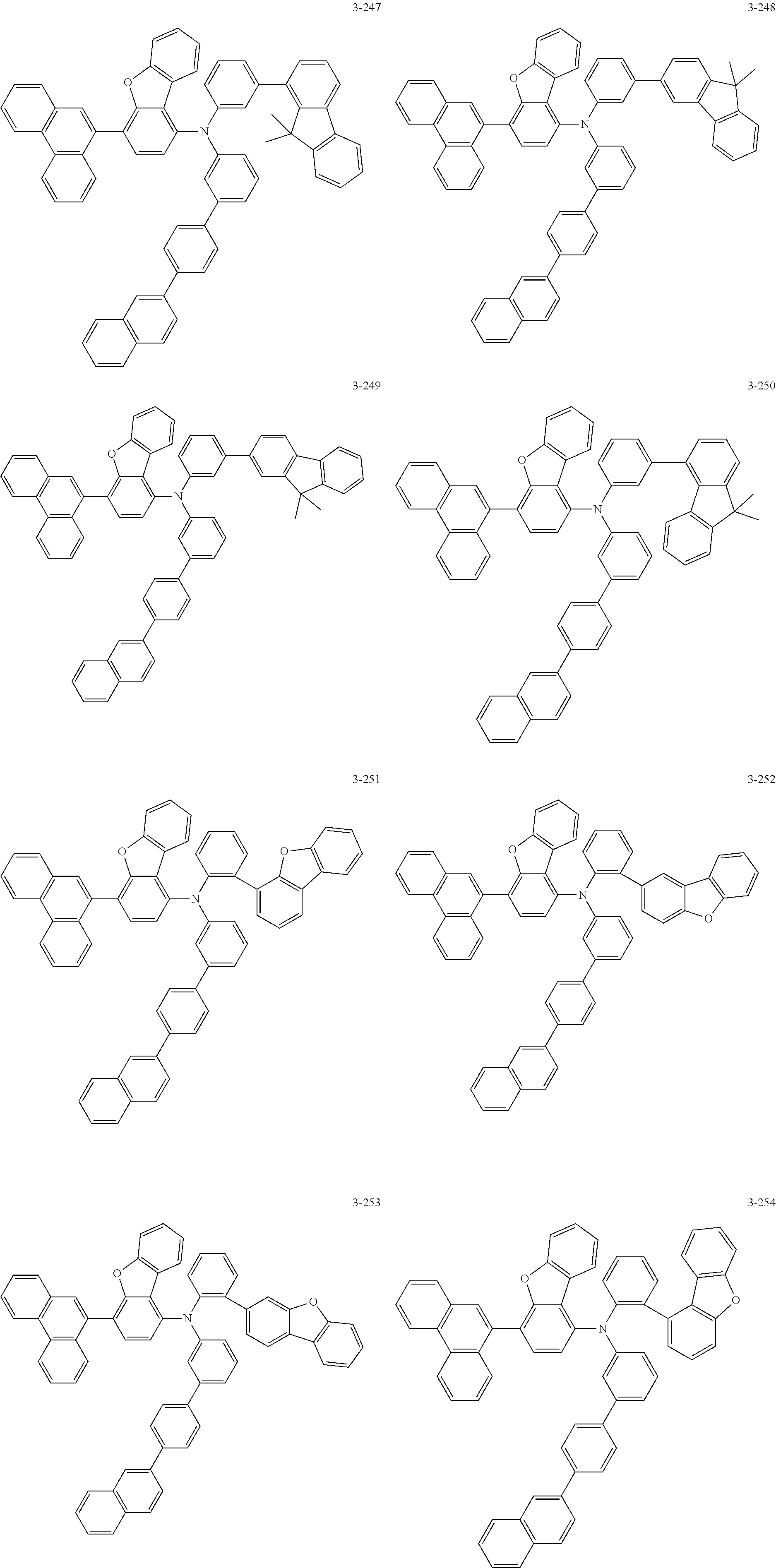

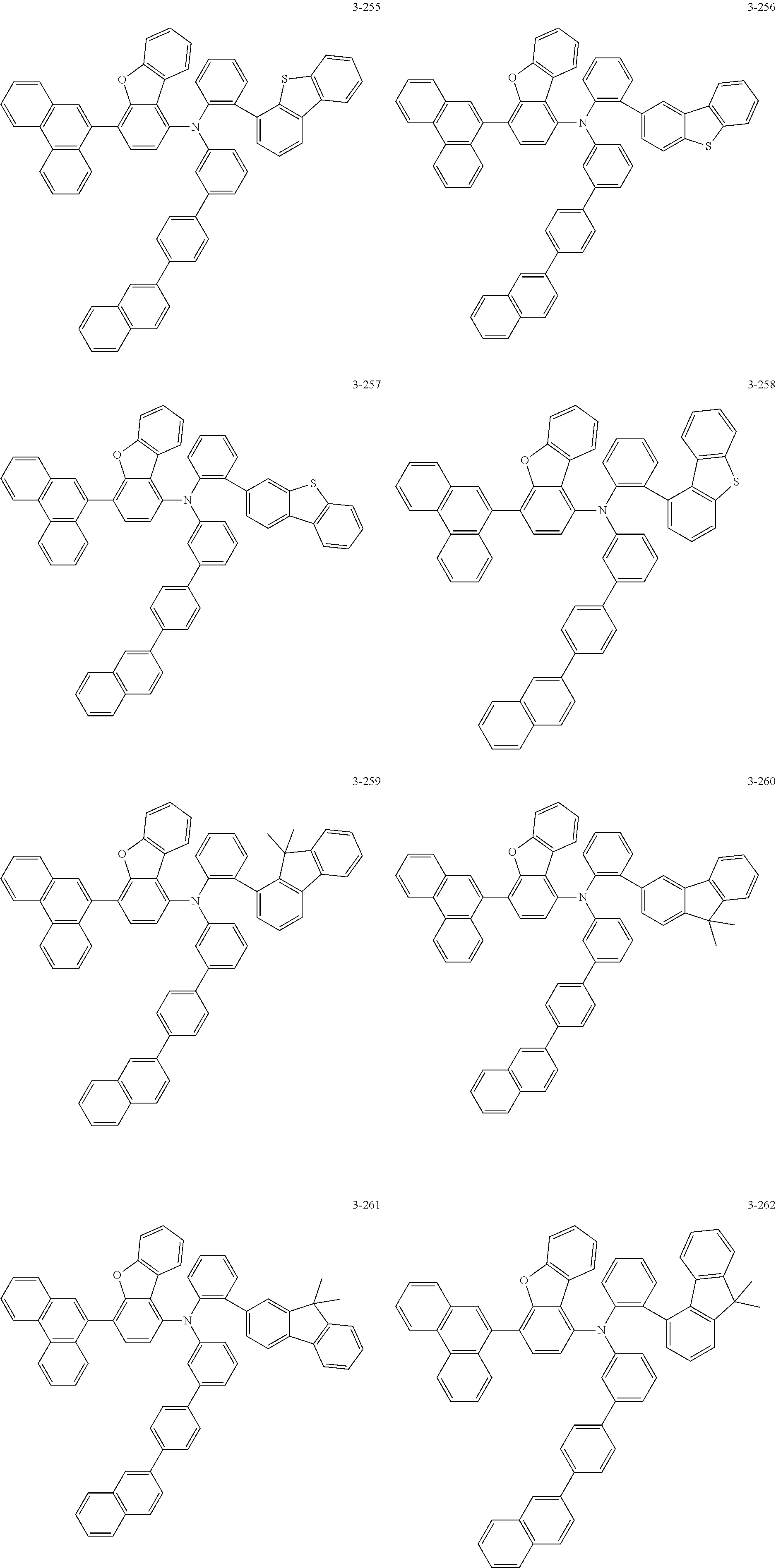

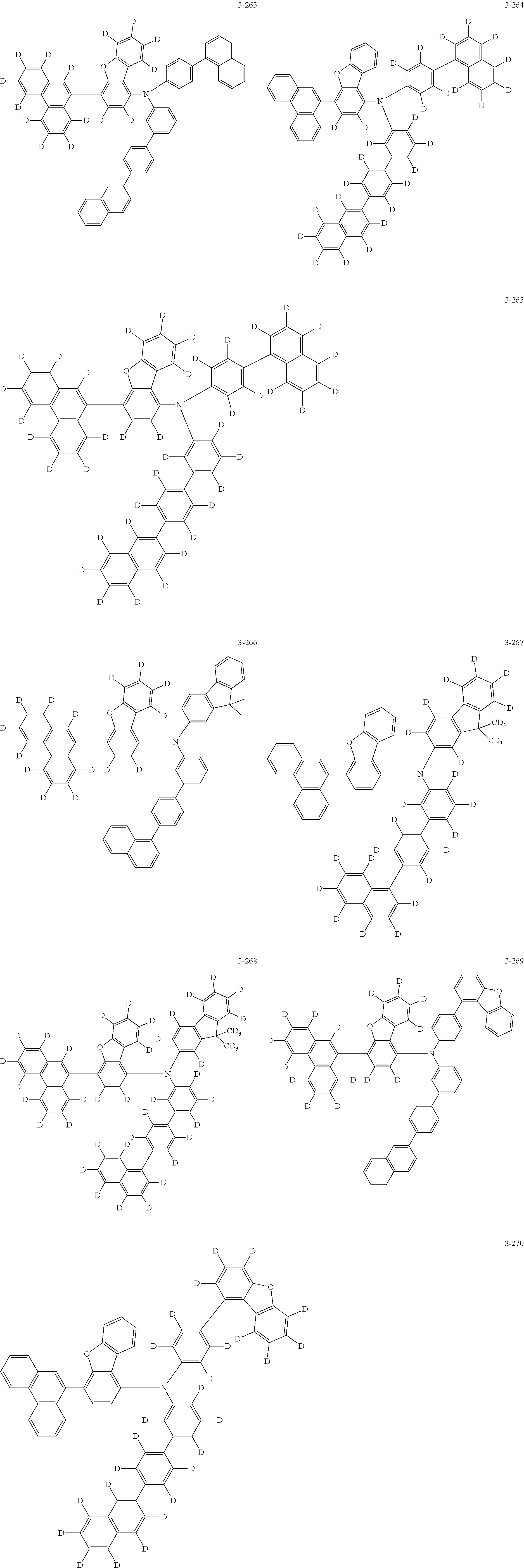

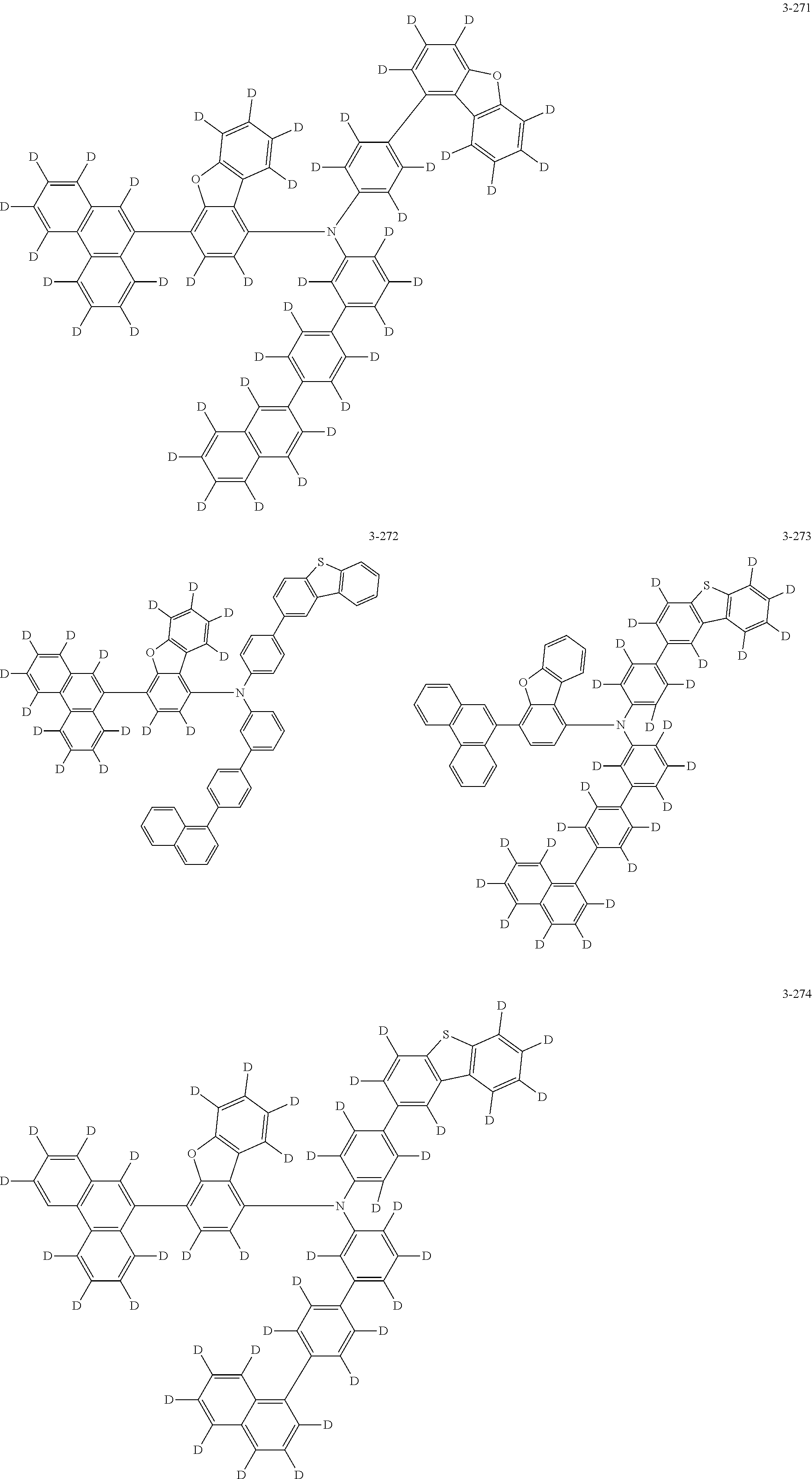

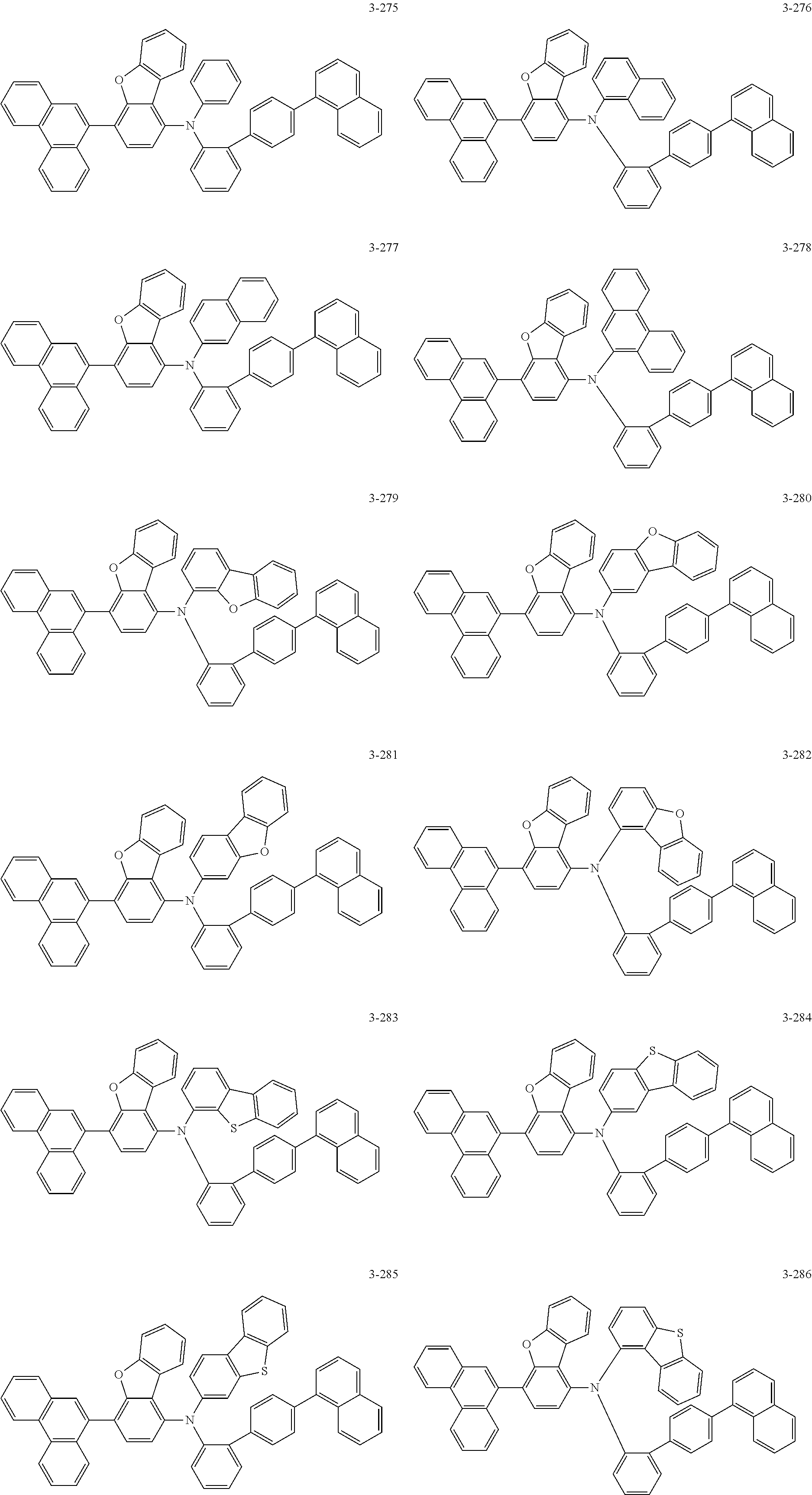

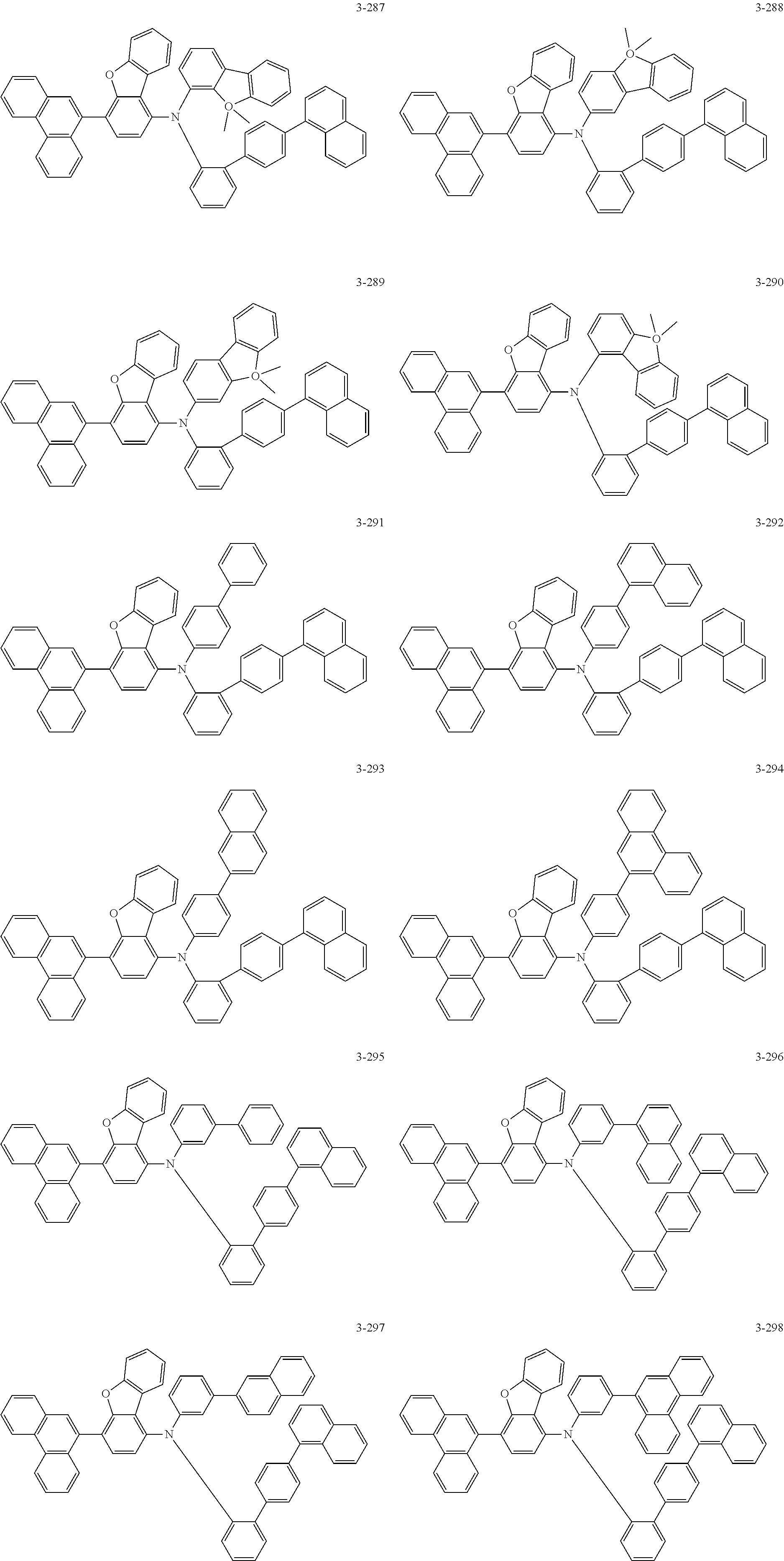

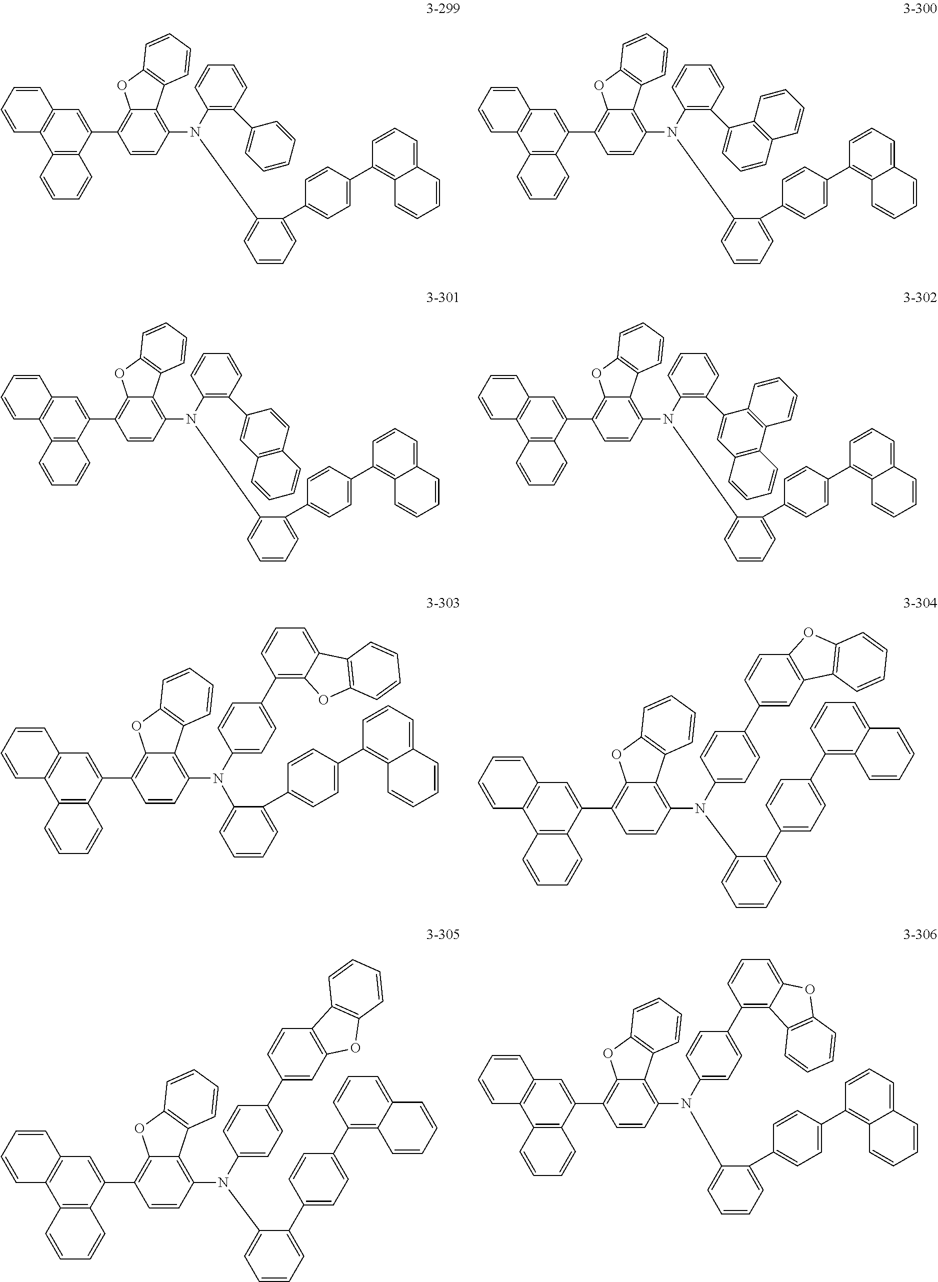

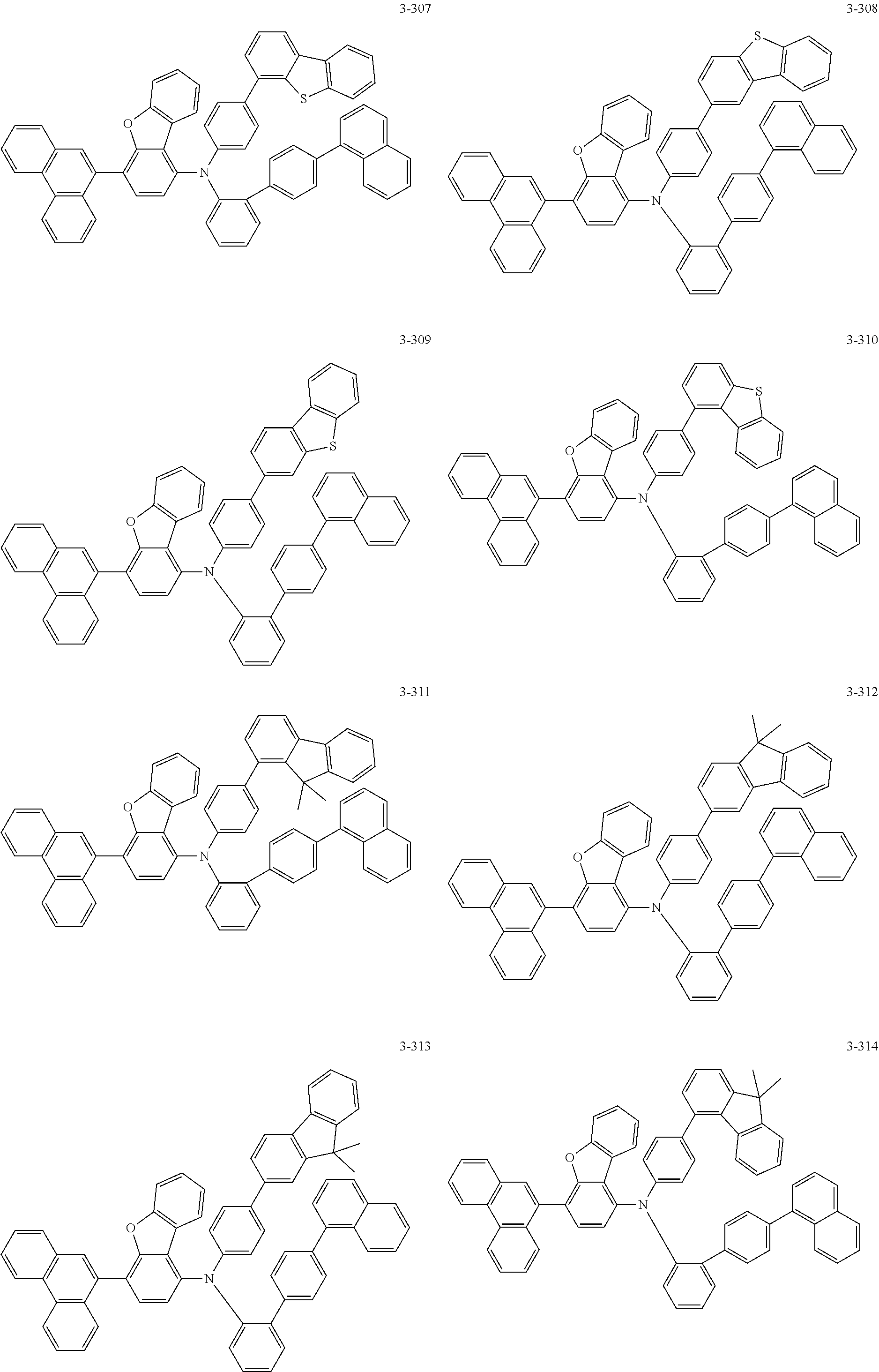

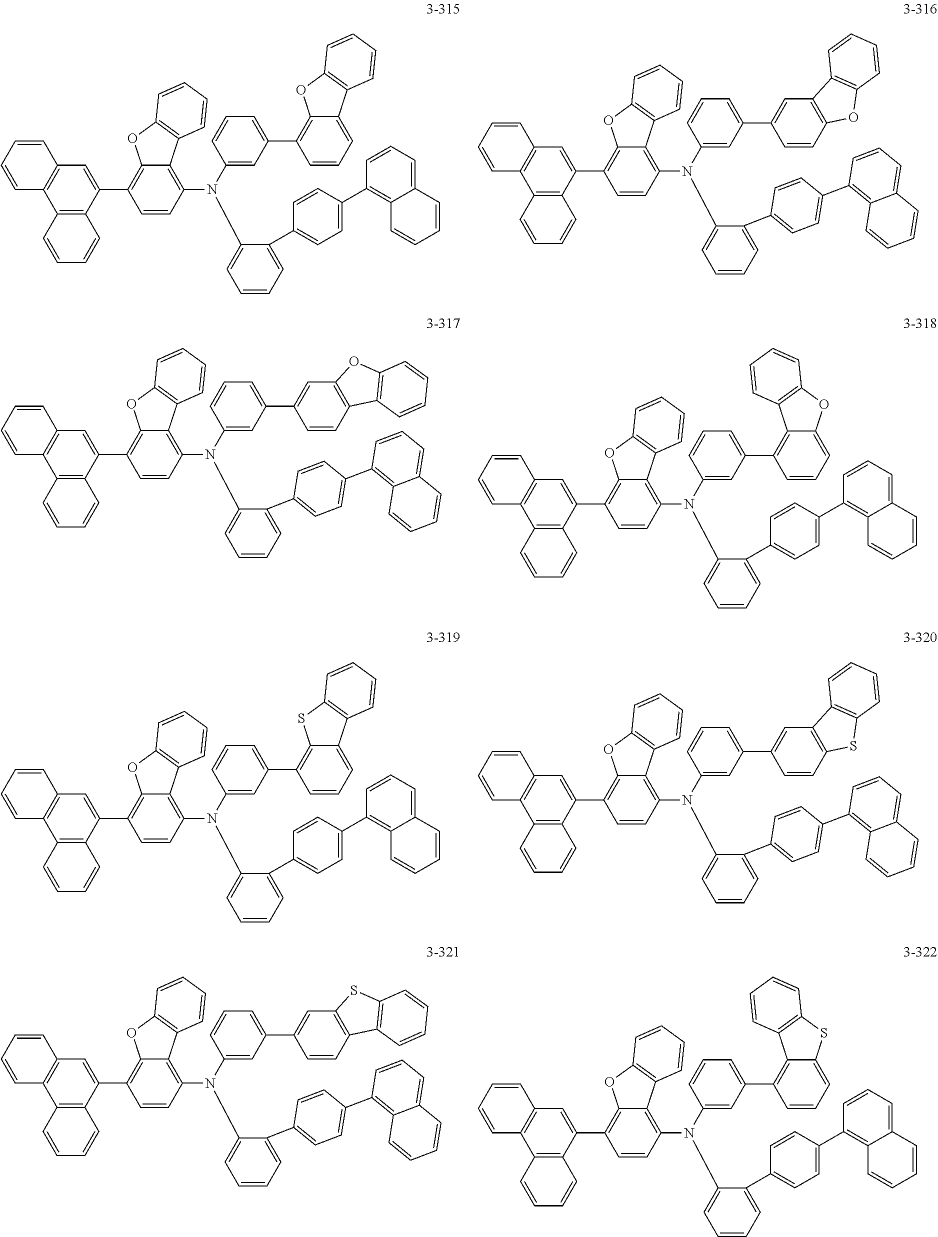

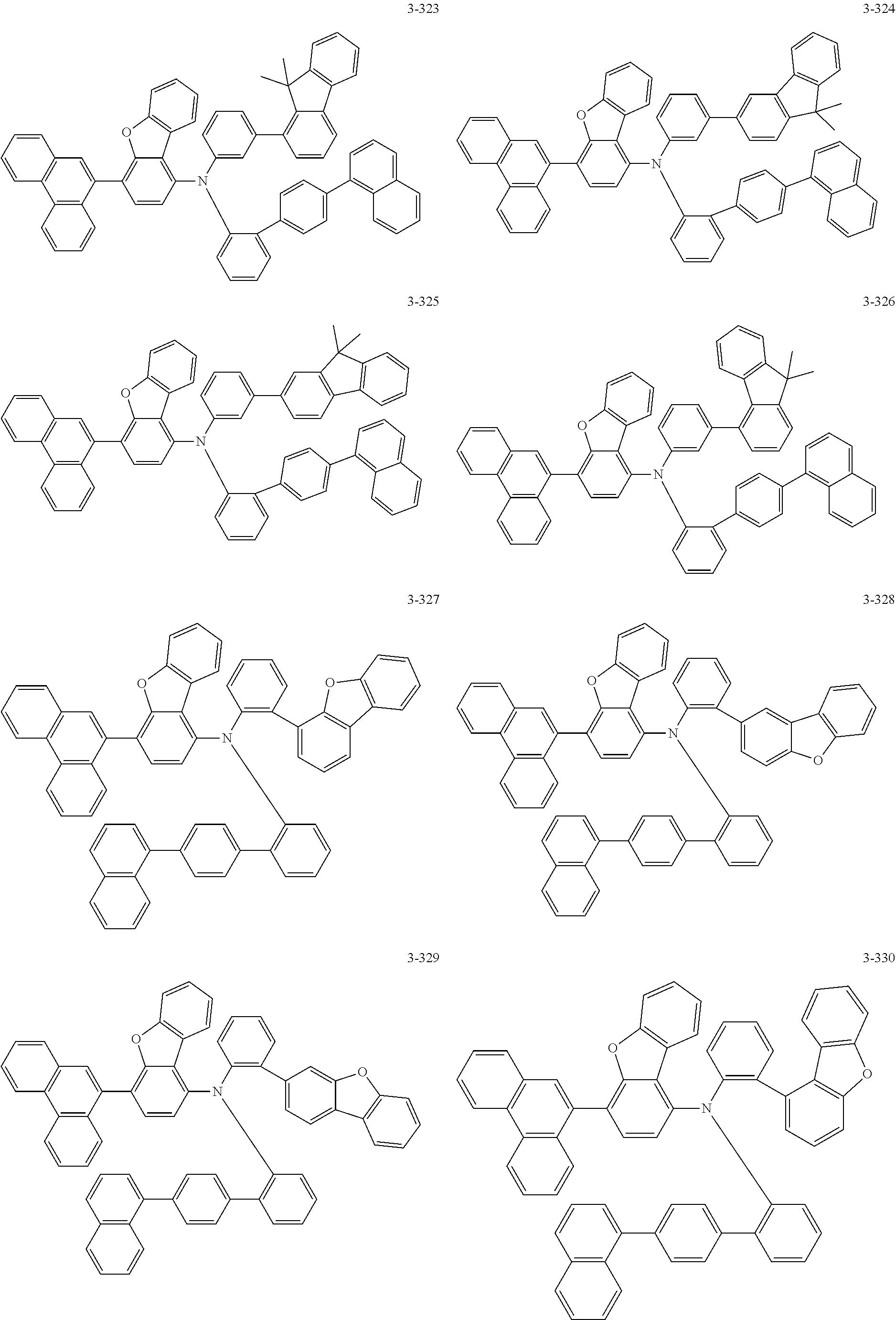

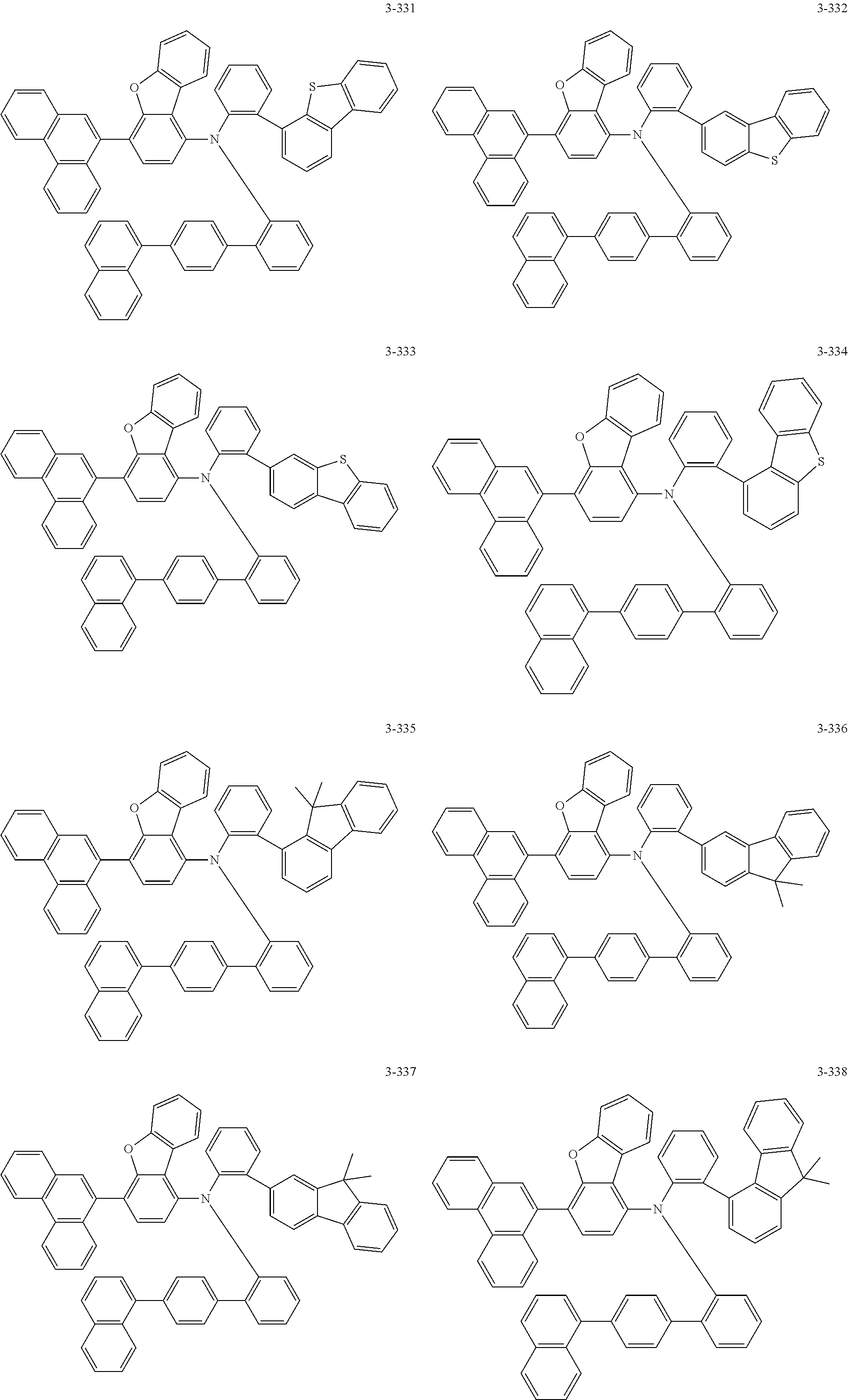

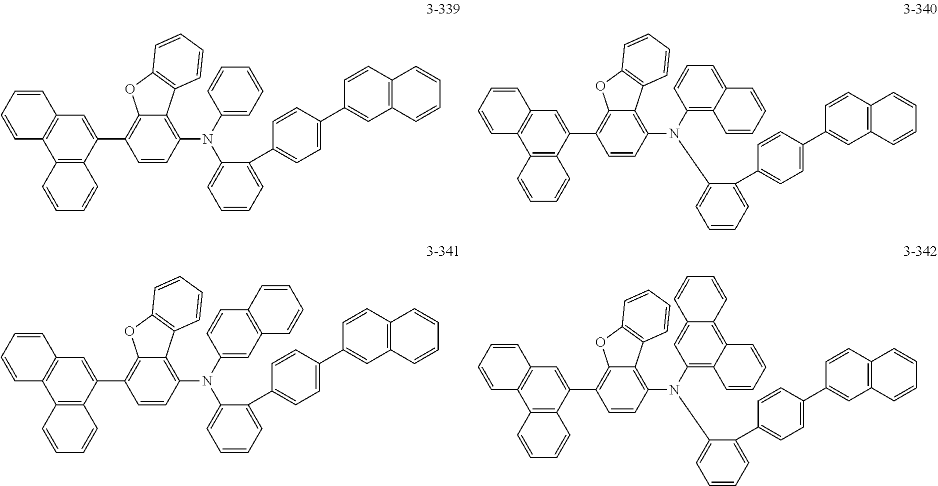 [SMILES: c1ccc(N(c2ccccc2-c2ccc(-c3ccc4ccccc4c3)cc2)c2ccc(-c3cc4ccccc4c4ccccc34)c3oc4ccccc4c23)cc1 ; c1ccc(N(c2cccc3ccccc23)c2ccc(-c3cc4ccccc4c4ccccc34)c3oc4ccccc4c23)c(-c2ccc(-c3ccc4ccccc4c3)cc2)c1 ; c1ccc(N(c2ccc3ccccc3c2)c2ccc(-c3cc4ccccc4c4ccccc34)c3oc4ccccc4c23)c(-c2ccc(-c3ccc4ccccc4c3)cc2)c1 ; c1ccc(N(c2cc3ccccc3c3ccccc23)c2ccc(-c3cc4ccccc4c4ccccc34)c3oc4ccccc4c23)c(-c2ccc(-c3ccc4ccccc4c3)cc2)c1]

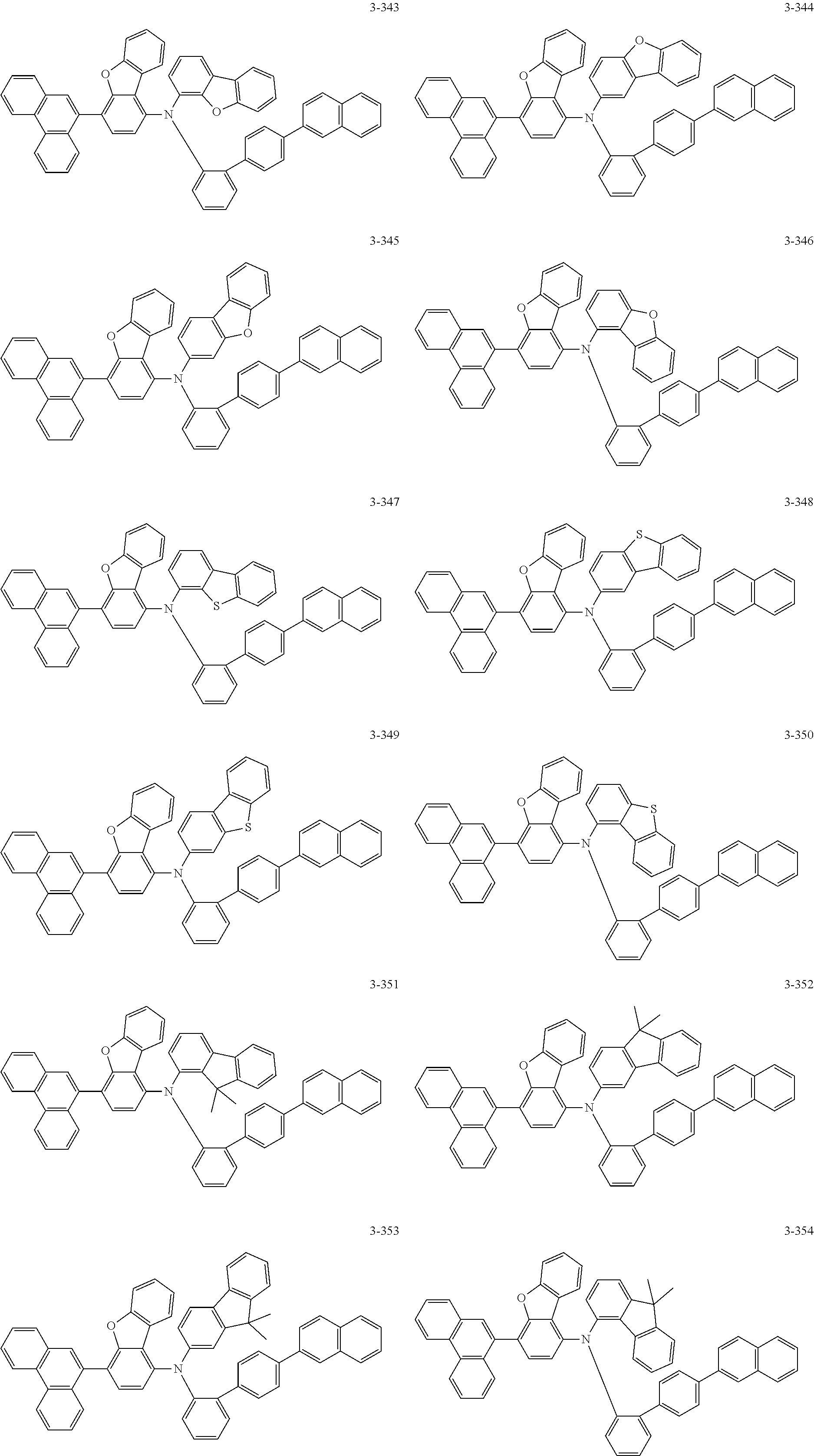

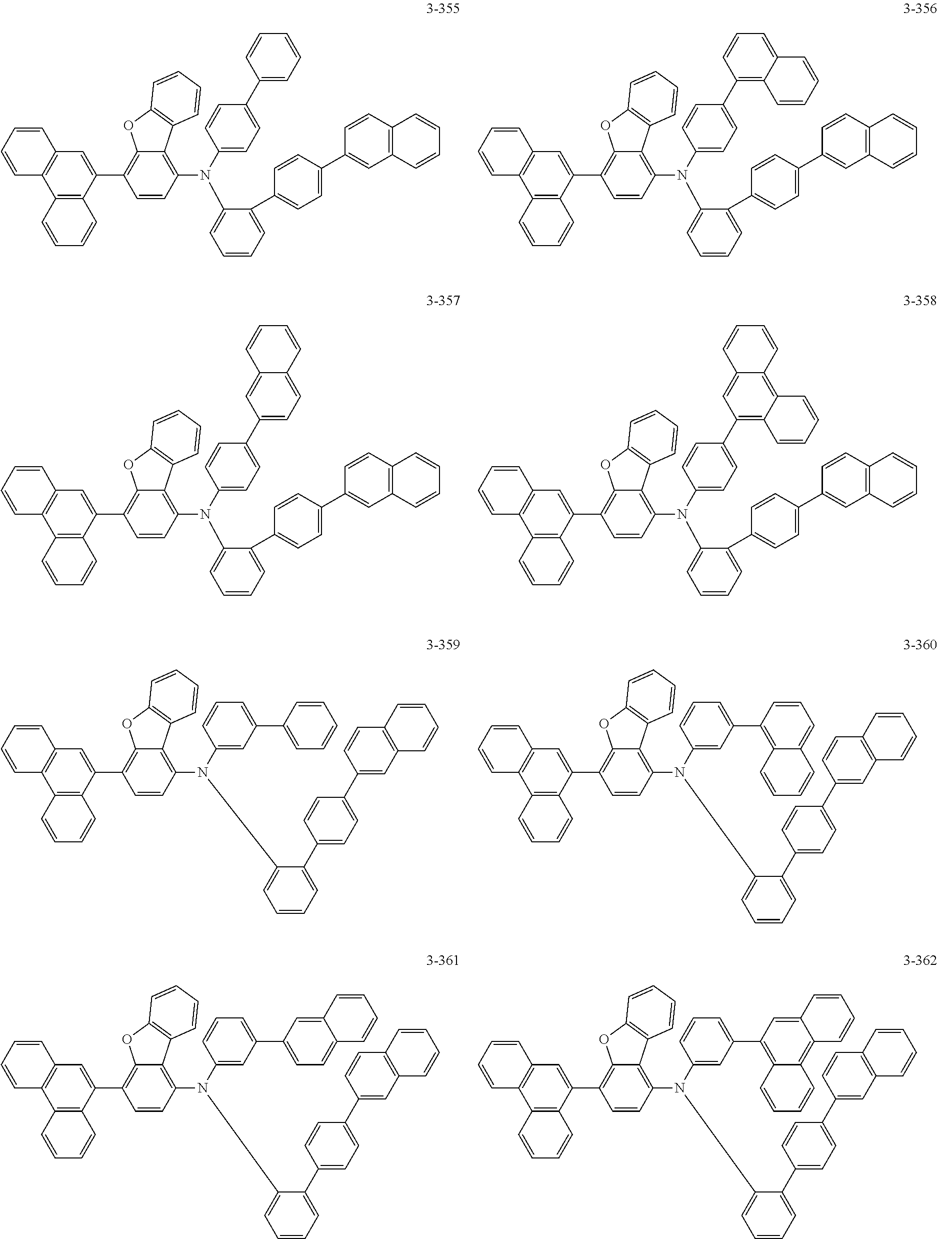

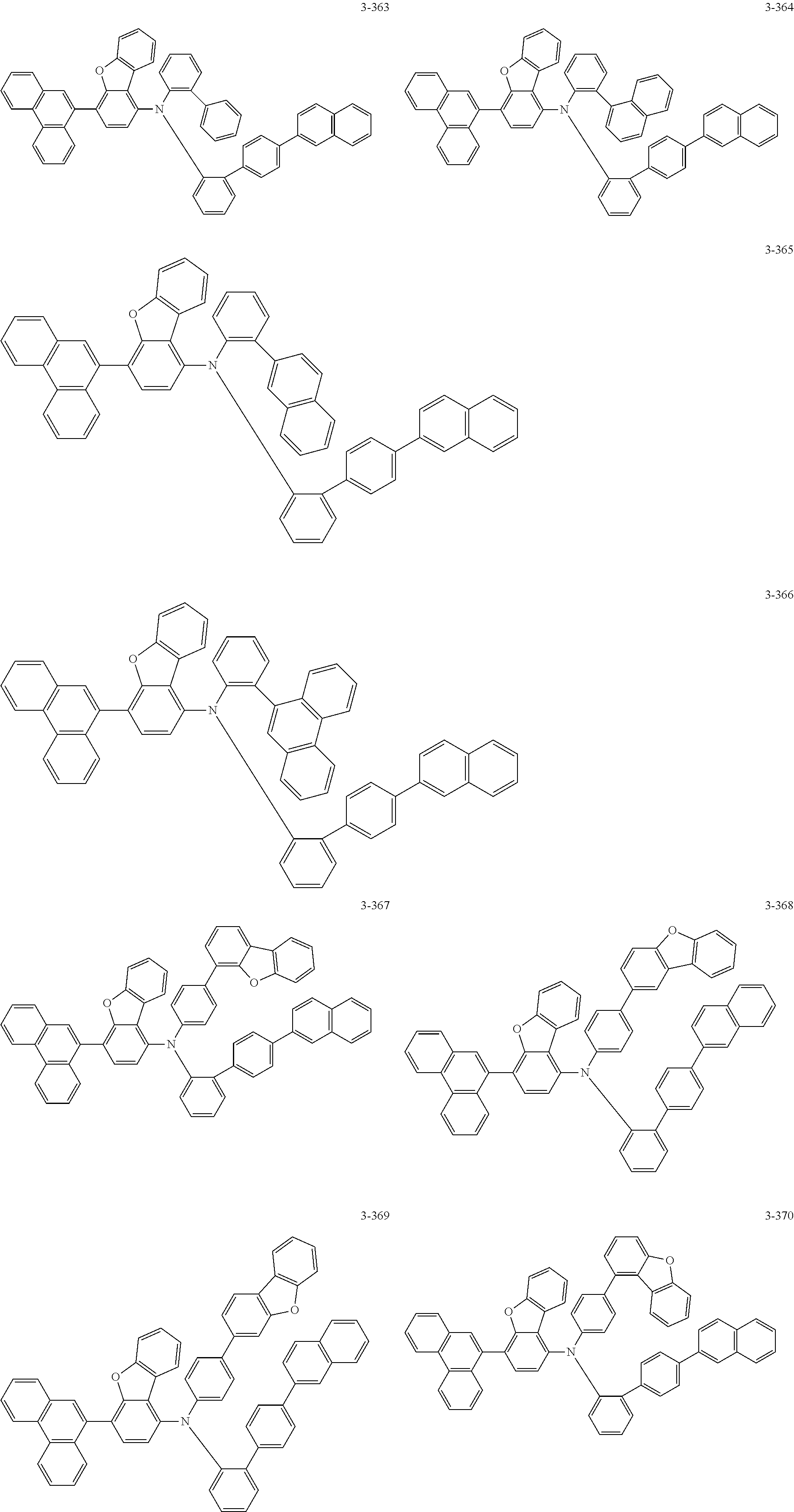

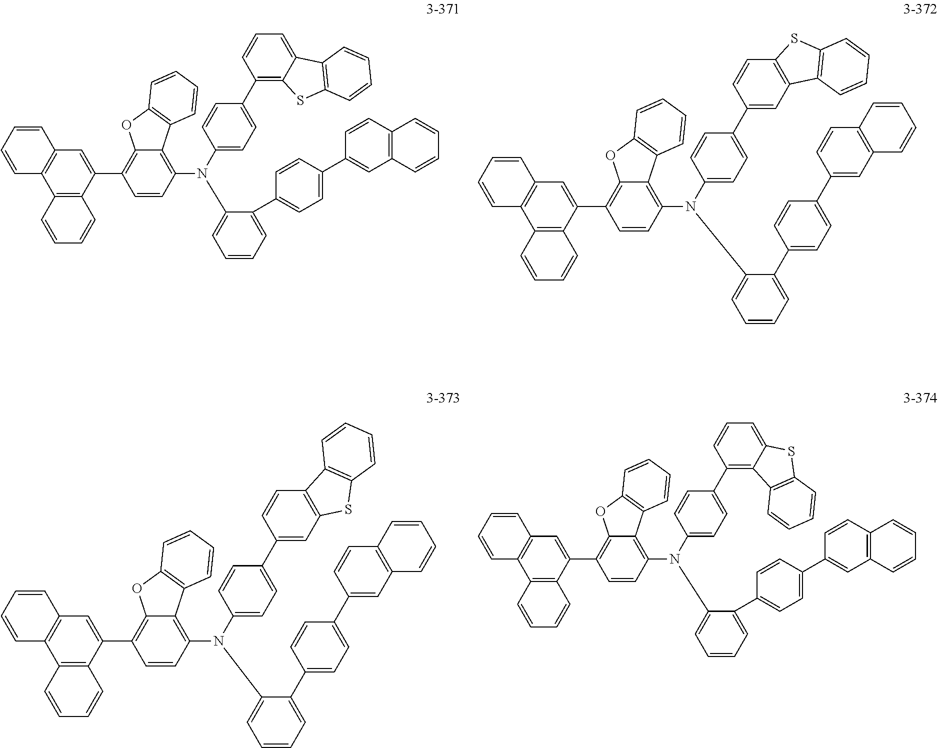 [SMILES: c1ccc(N(c2ccc(-c3cccc4c3sc3ccccc34)cc2)c2ccc(-c3cc4ccccc4c4ccccc34)c3oc4ccccc4c23)c(-c2ccc(-c3ccc4ccccc4c3)cc2)c1 ; c1ccc(N(c2ccc(-c3ccc4sc5ccccc5c4c3)cc2)c2ccc(-c3cc4ccccc4c4ccccc34)c3oc4ccccc4c23)c(-c2ccc(-c3ccc4ccccc4c3)cc2)c1 ; c1ccc(N(c2ccc(-c3ccc4c(c3)sc3ccccc34)cc2)c2ccc(-c3cc4ccccc4c4ccccc34)c3oc4ccccc4c23)c(-c2ccc(-c3ccc4ccccc4c3)cc2)c1 ; c1ccc(N(c2ccc(-c3cccc4sc5ccccc5c34)cc2)c2ccc(-c3cc4ccccc4c4ccccc34)c3oc4ccccc4c23)c(-c2ccc(-c3ccc4ccccc4c3)cc2)c1]

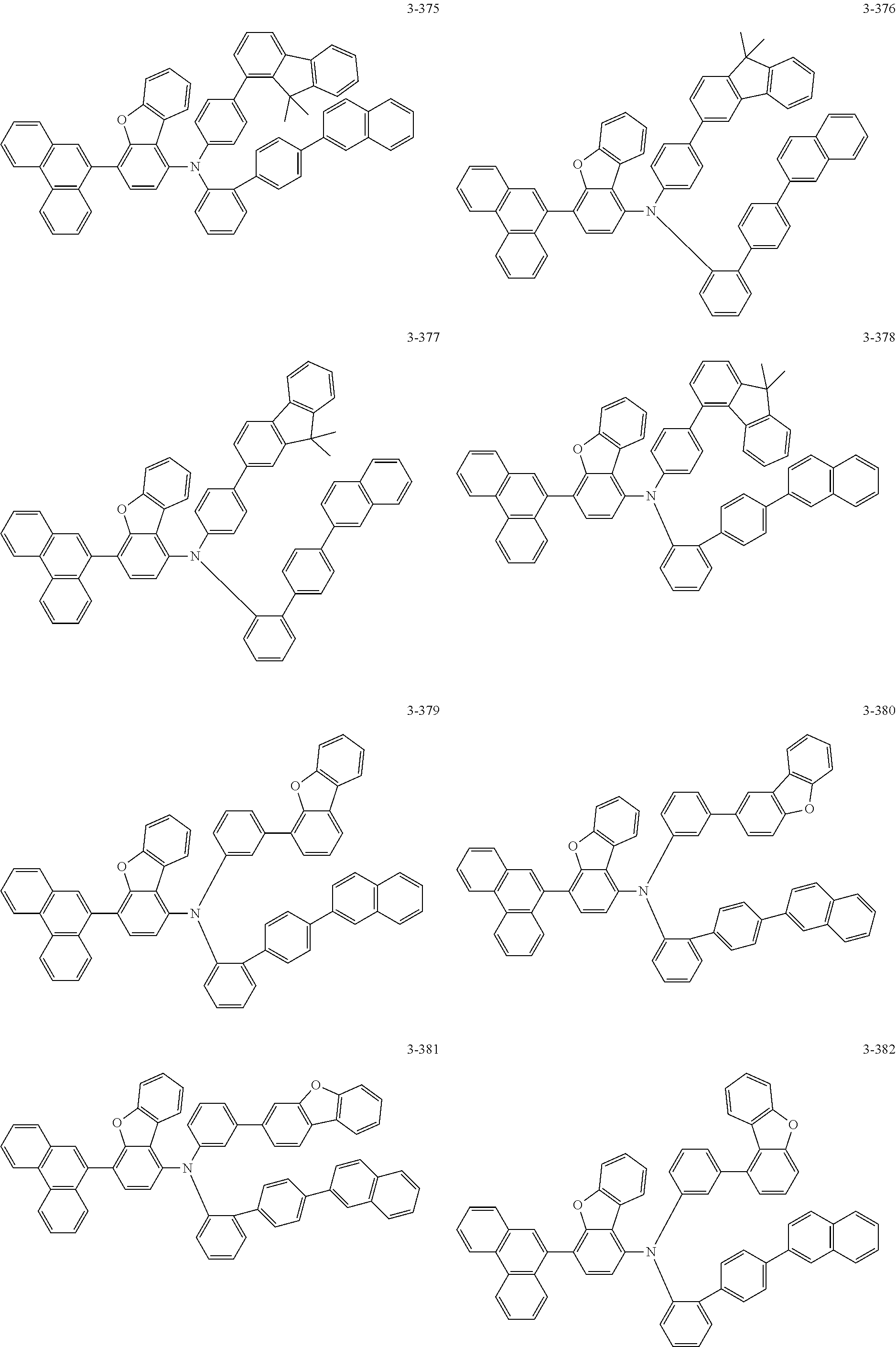

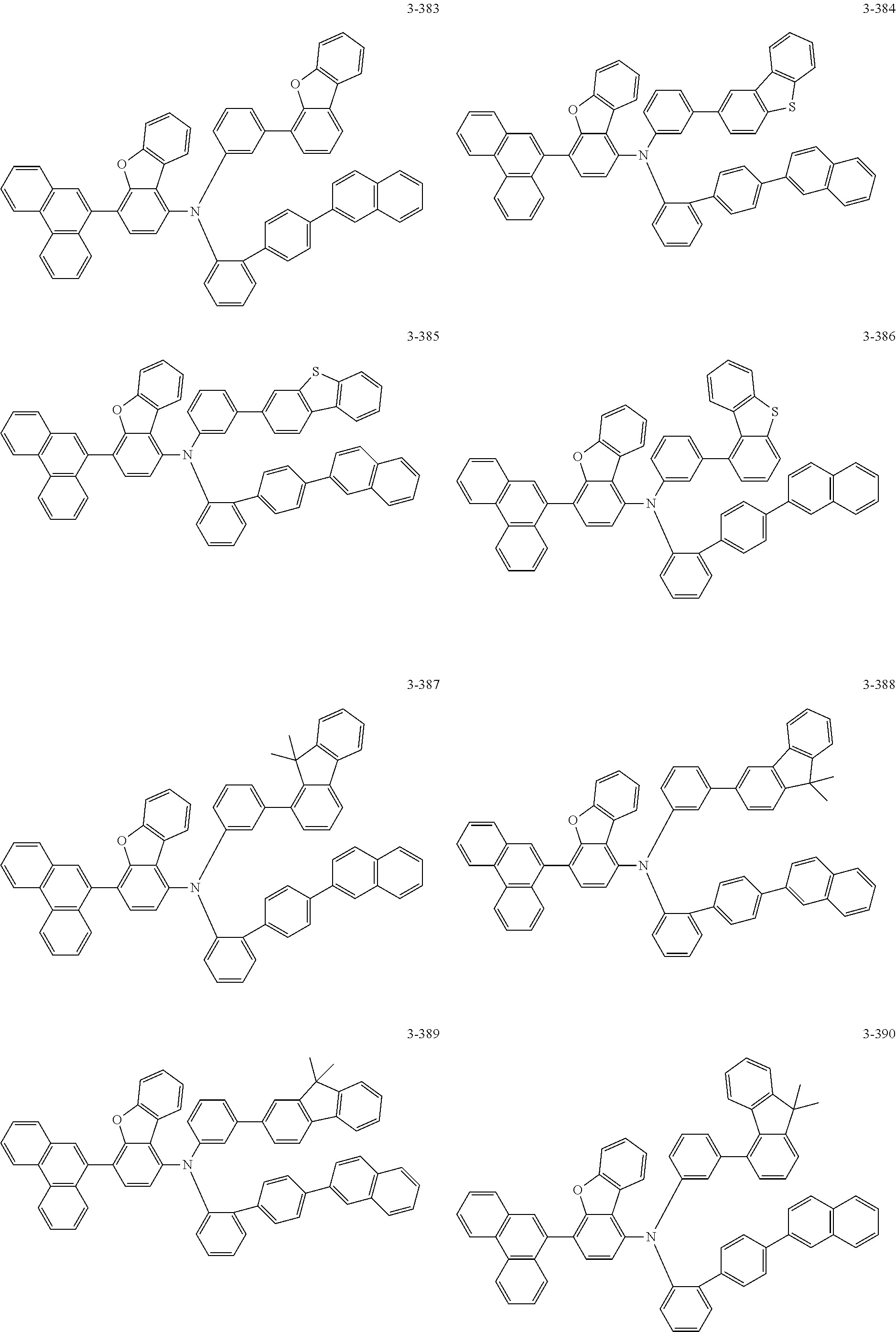

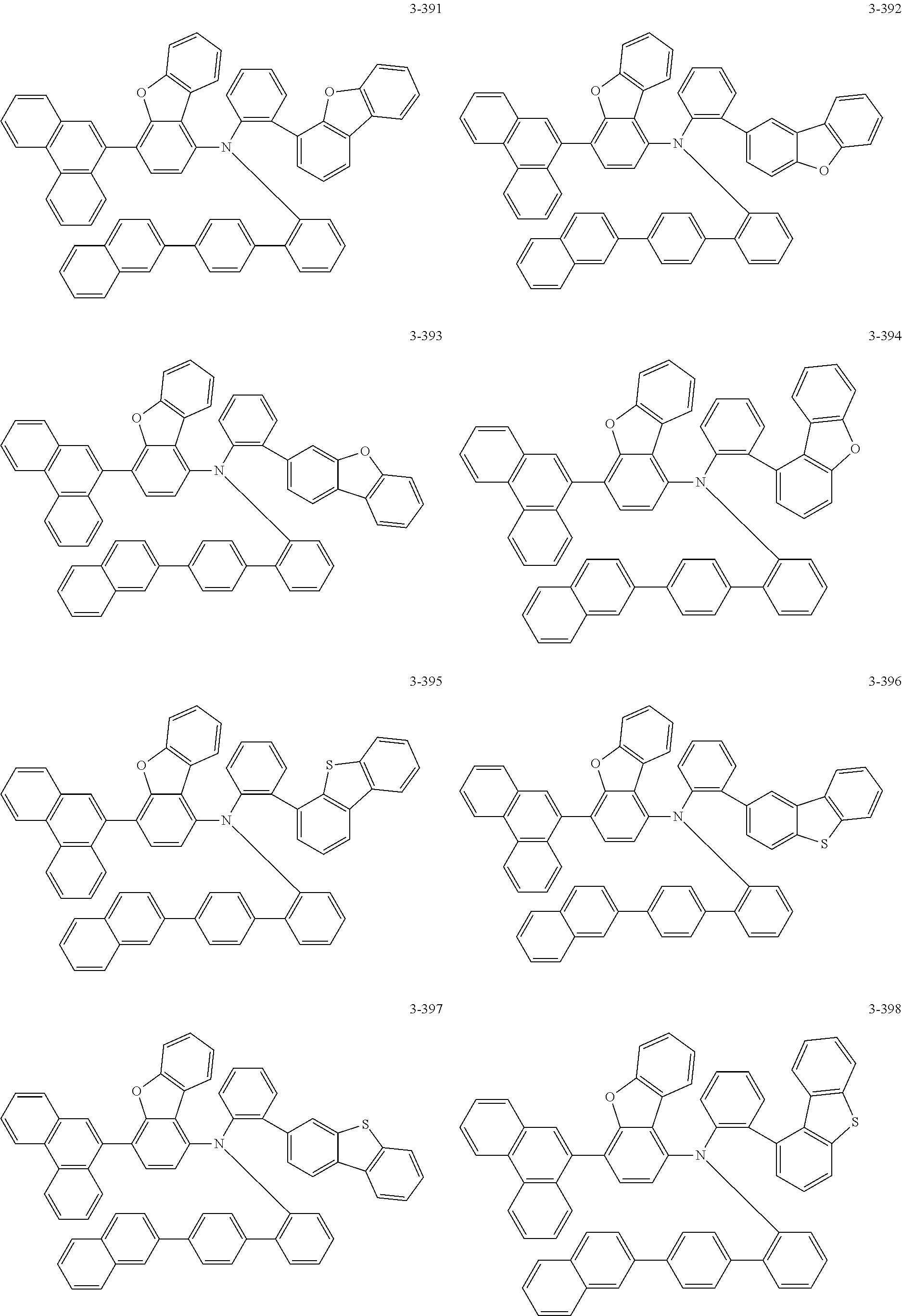

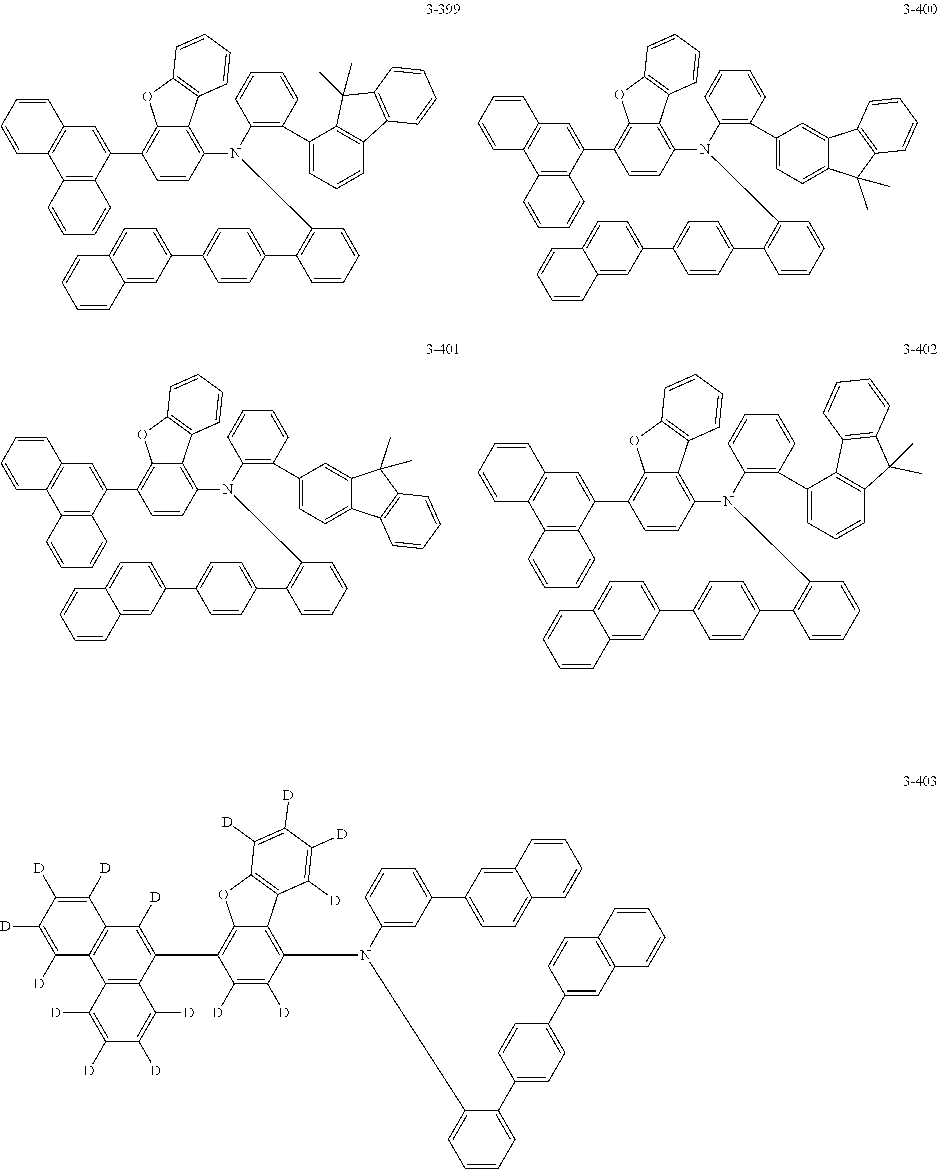

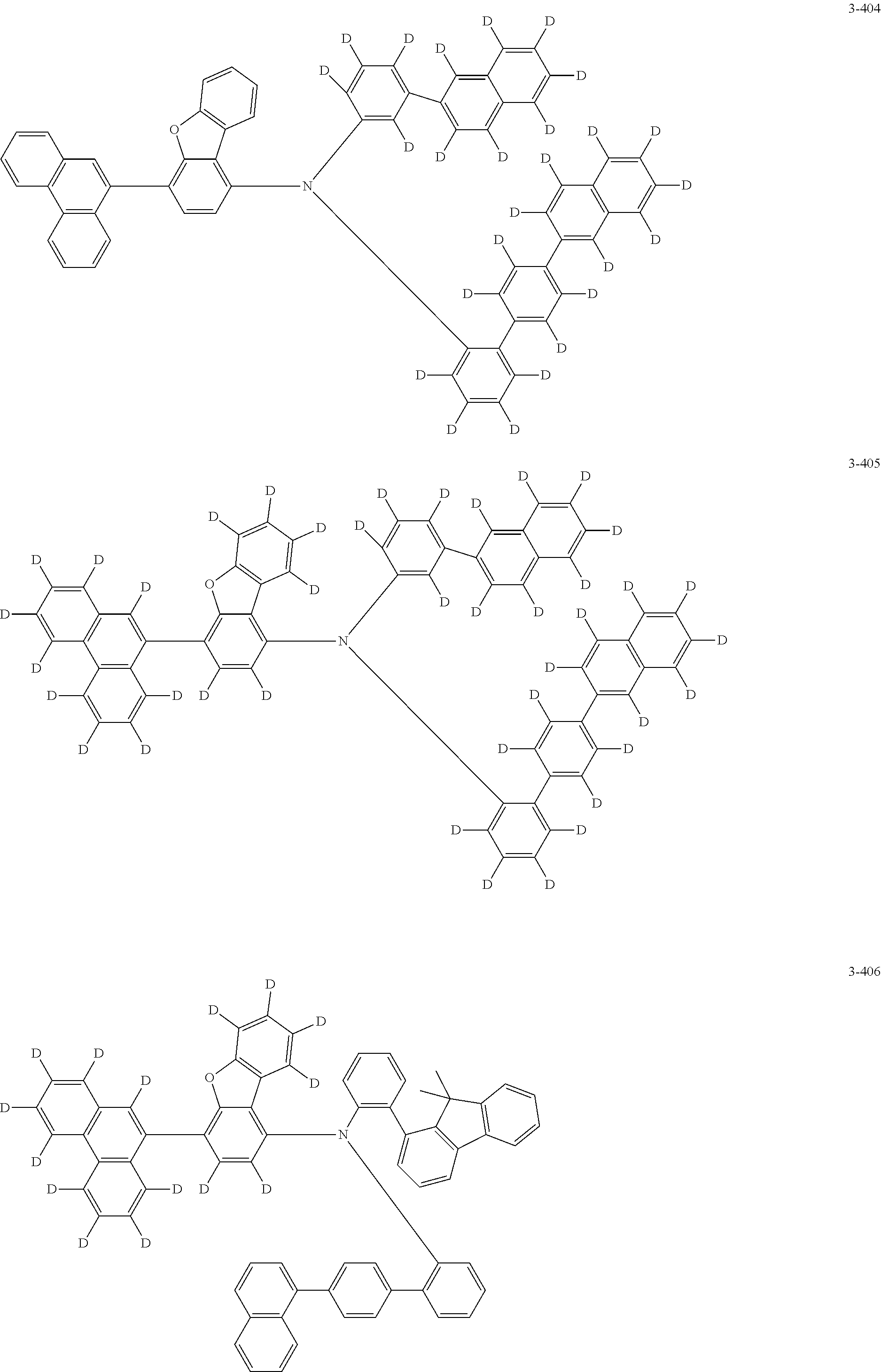

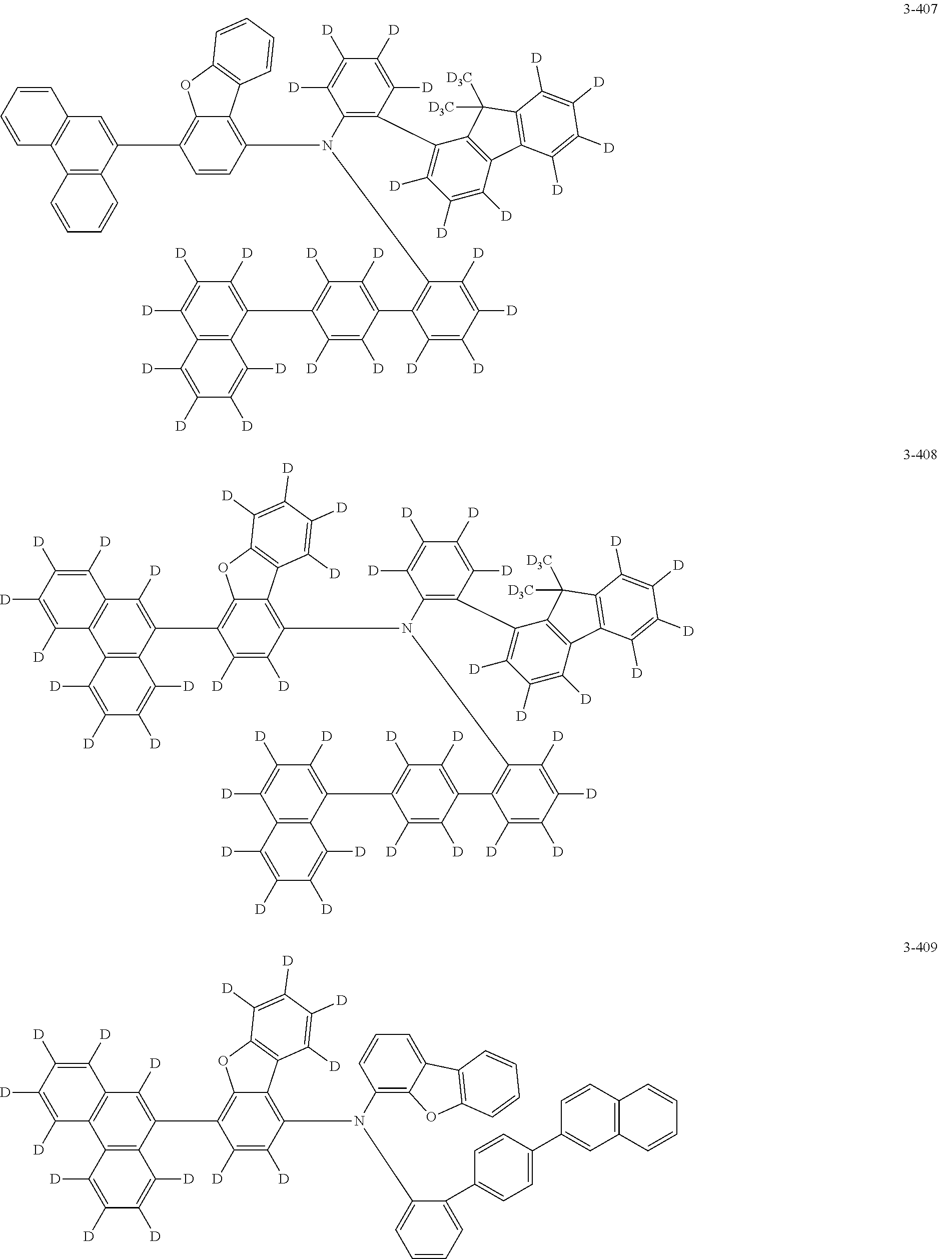

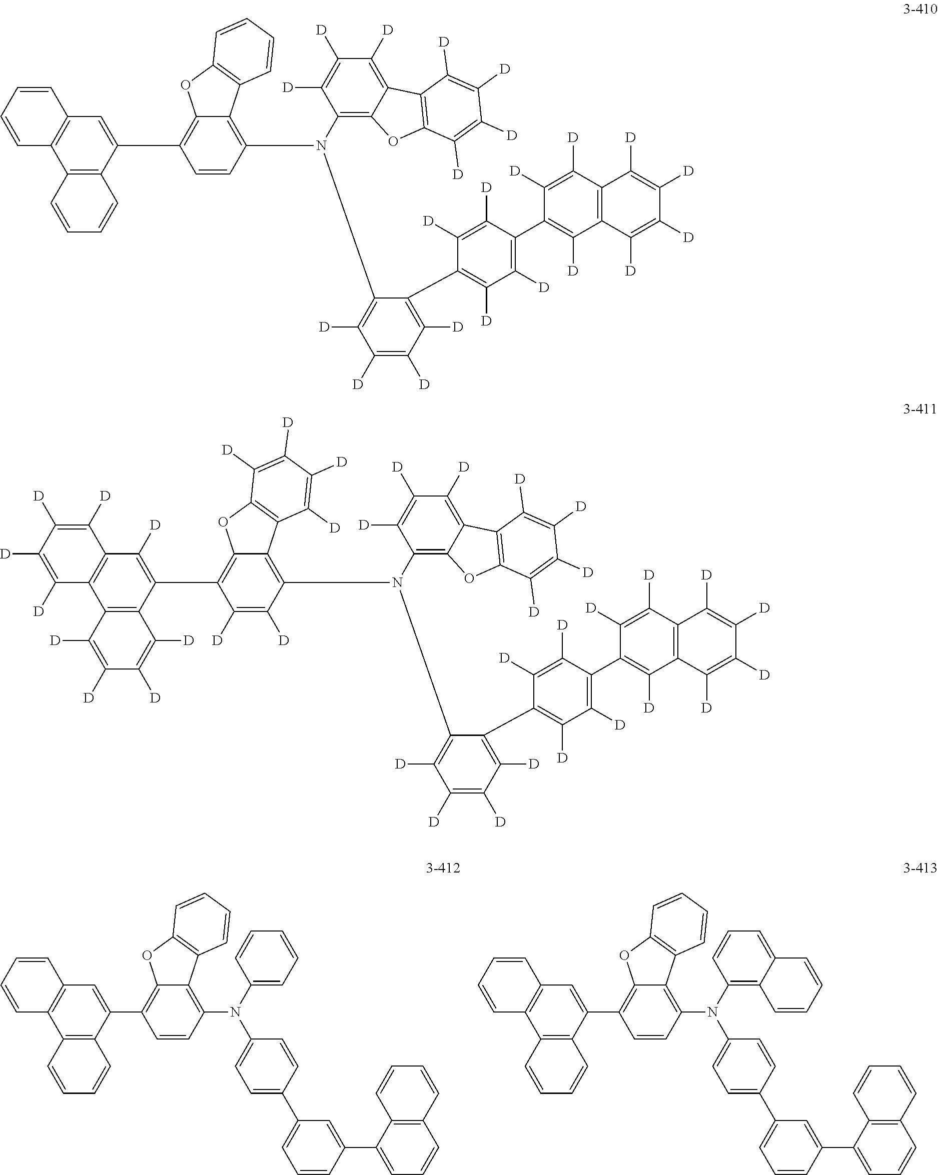

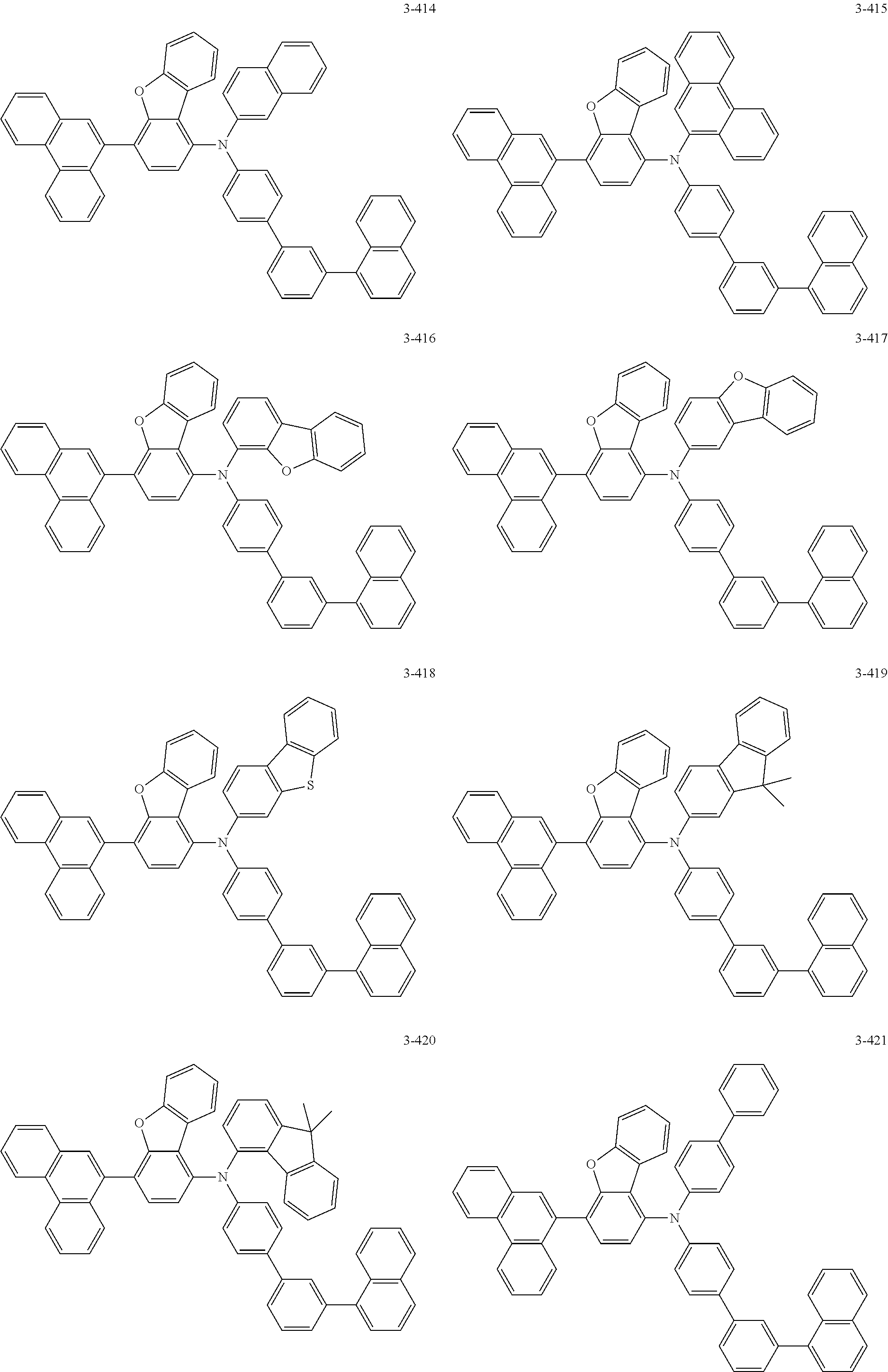

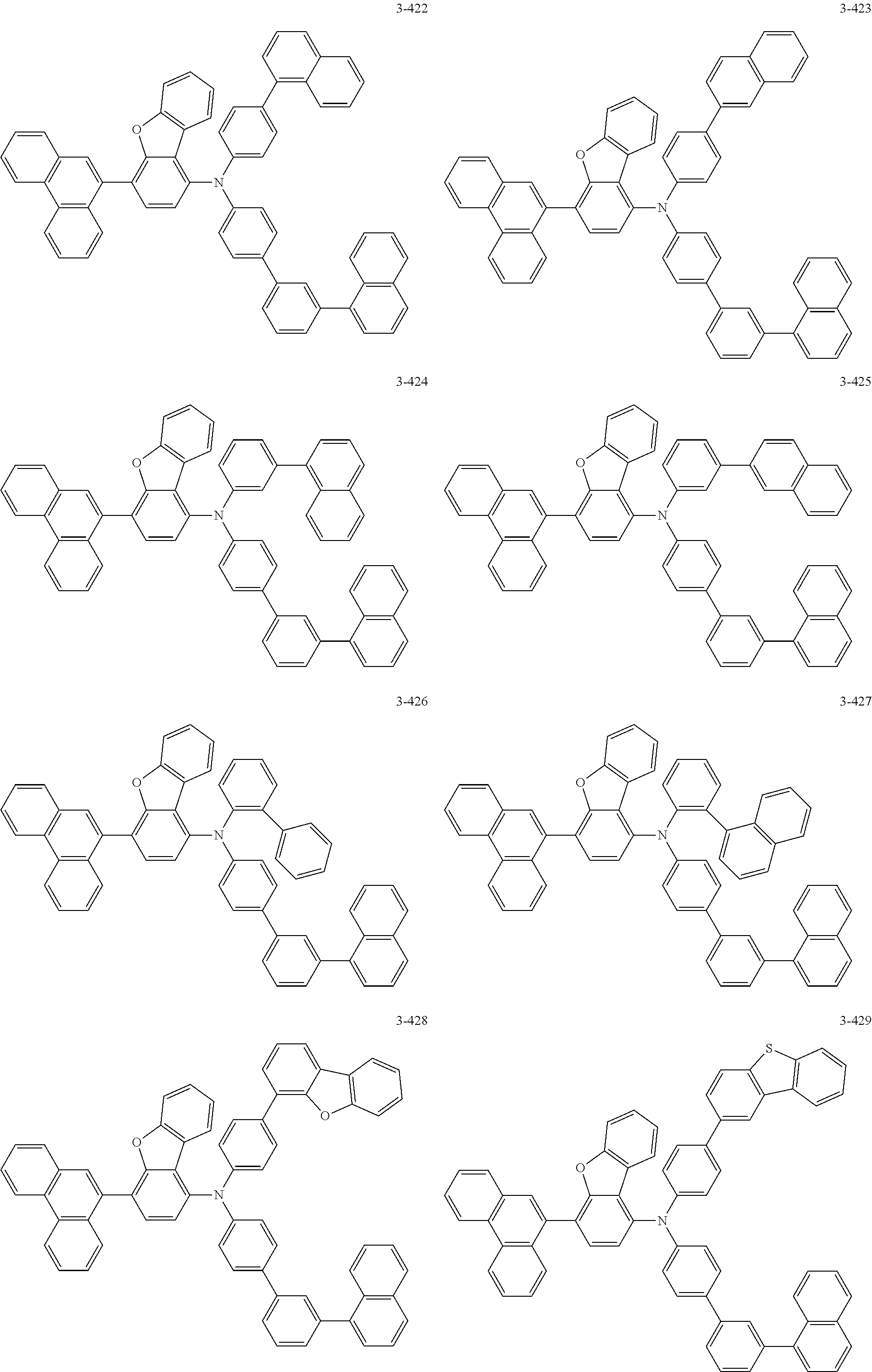

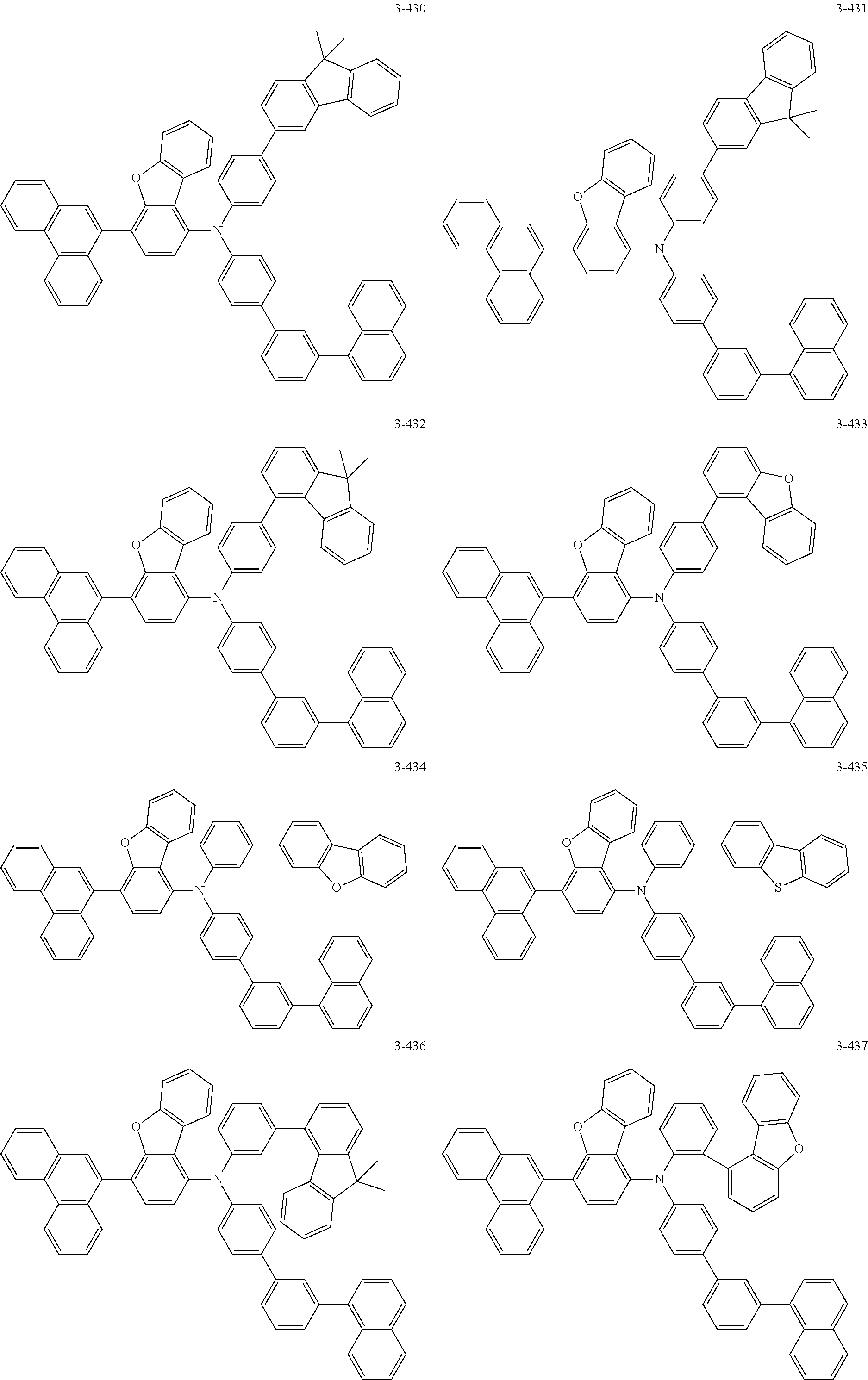

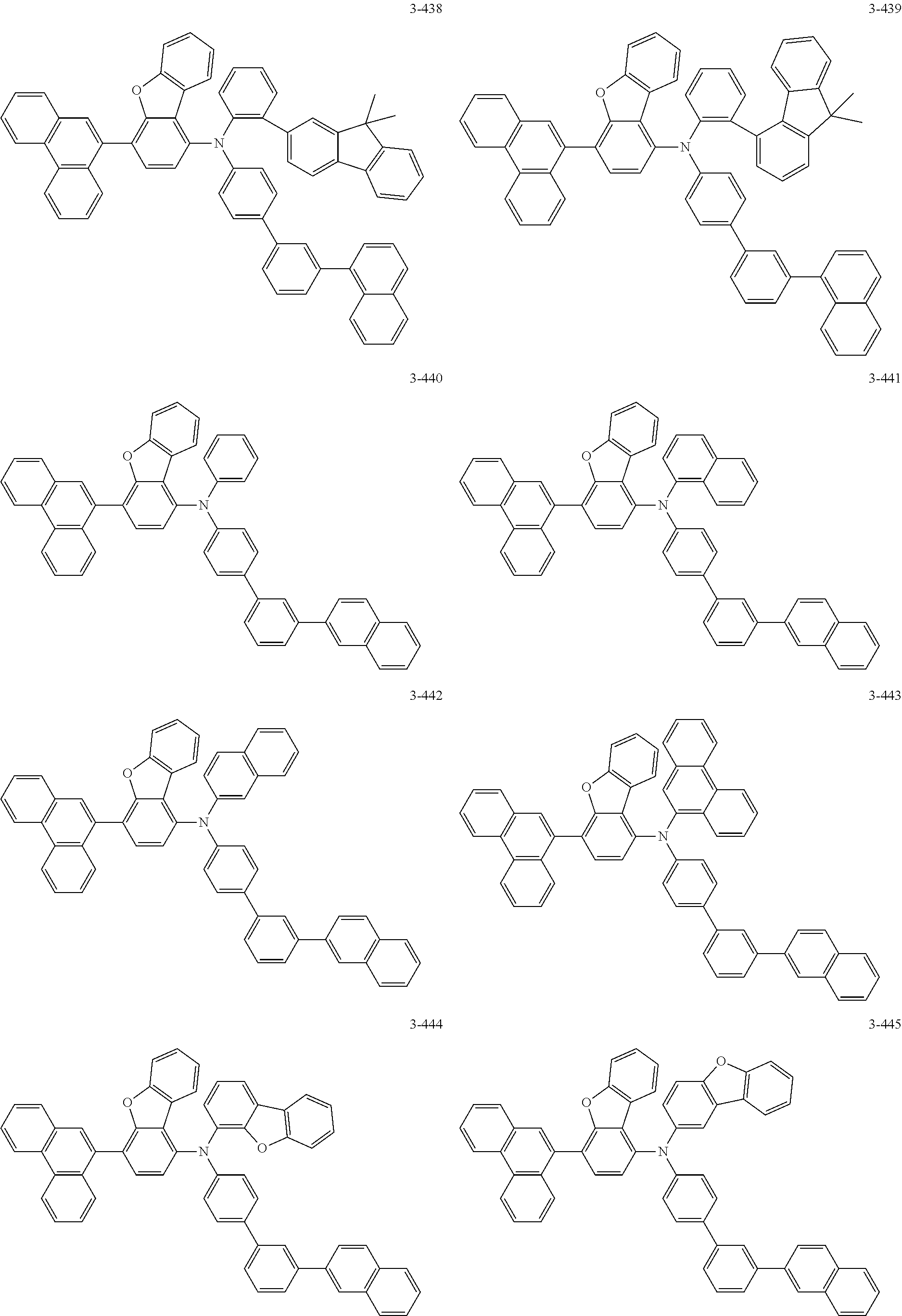

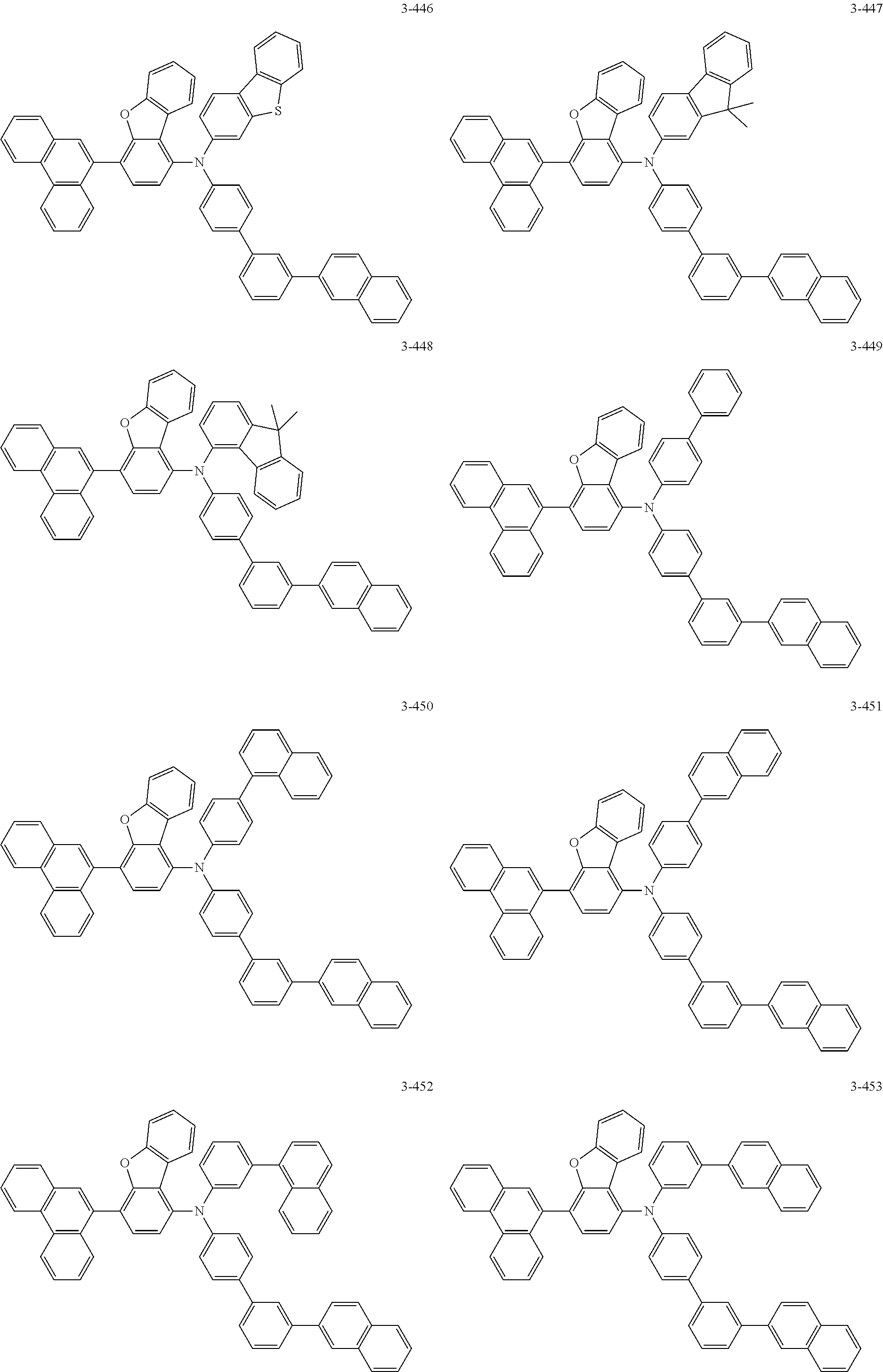

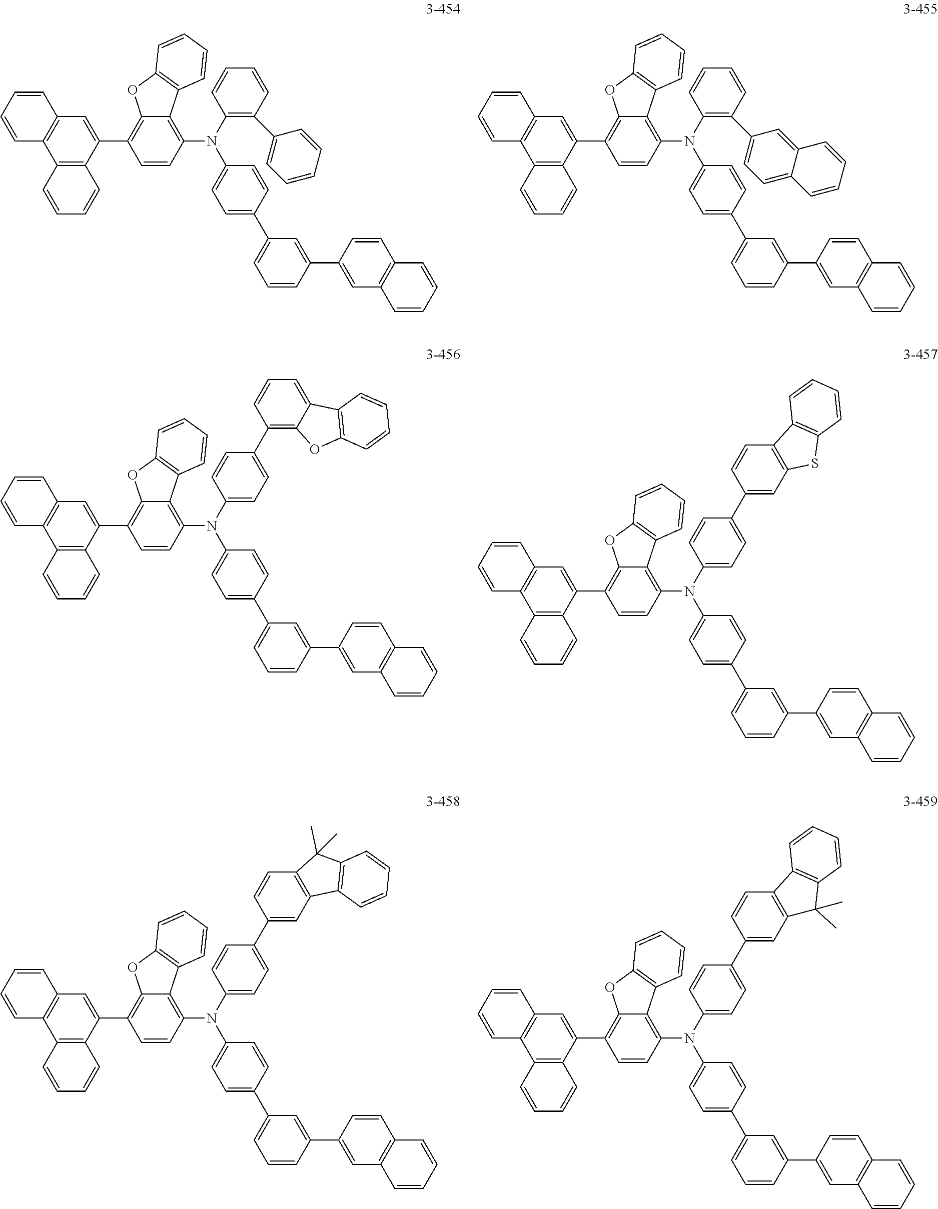

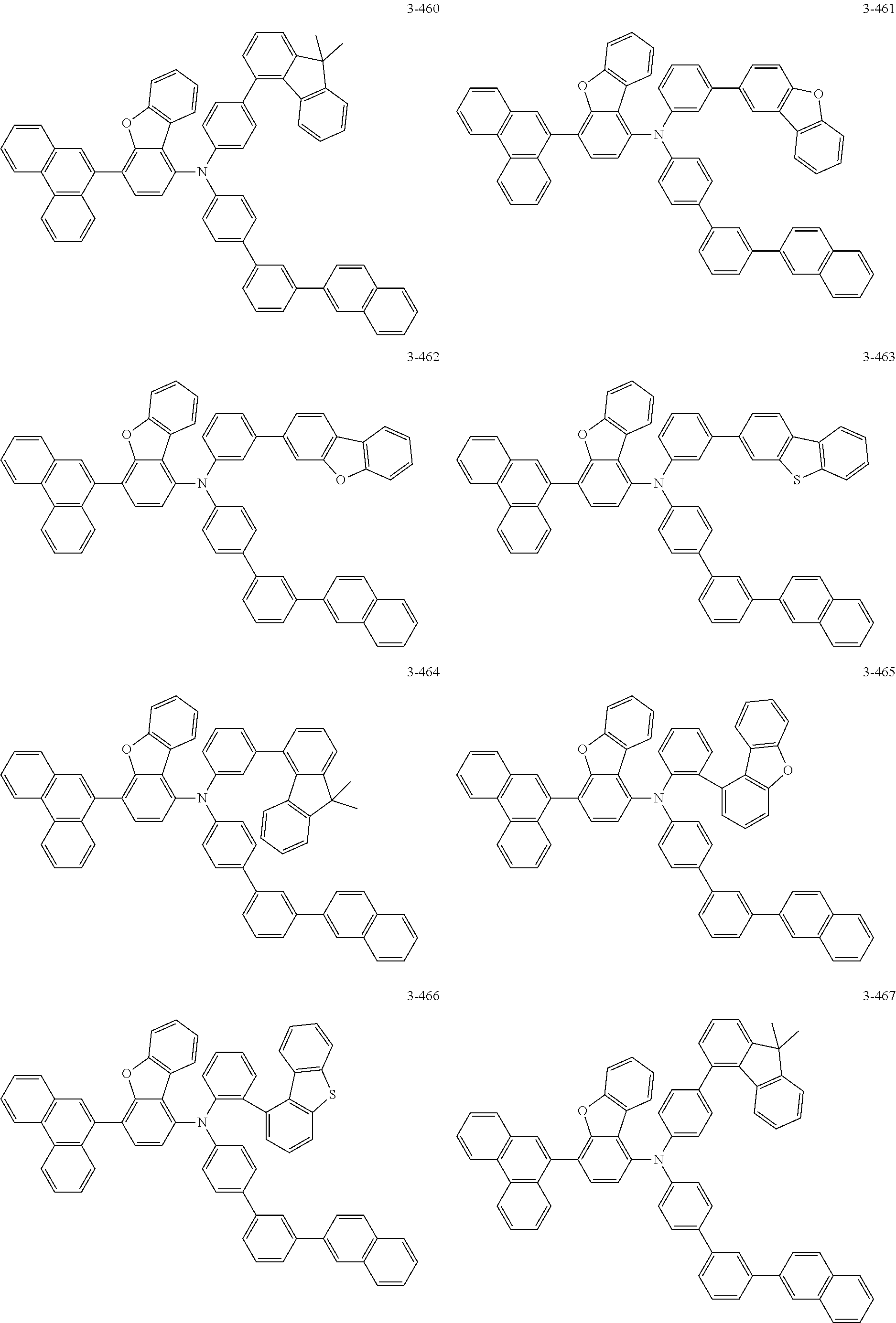

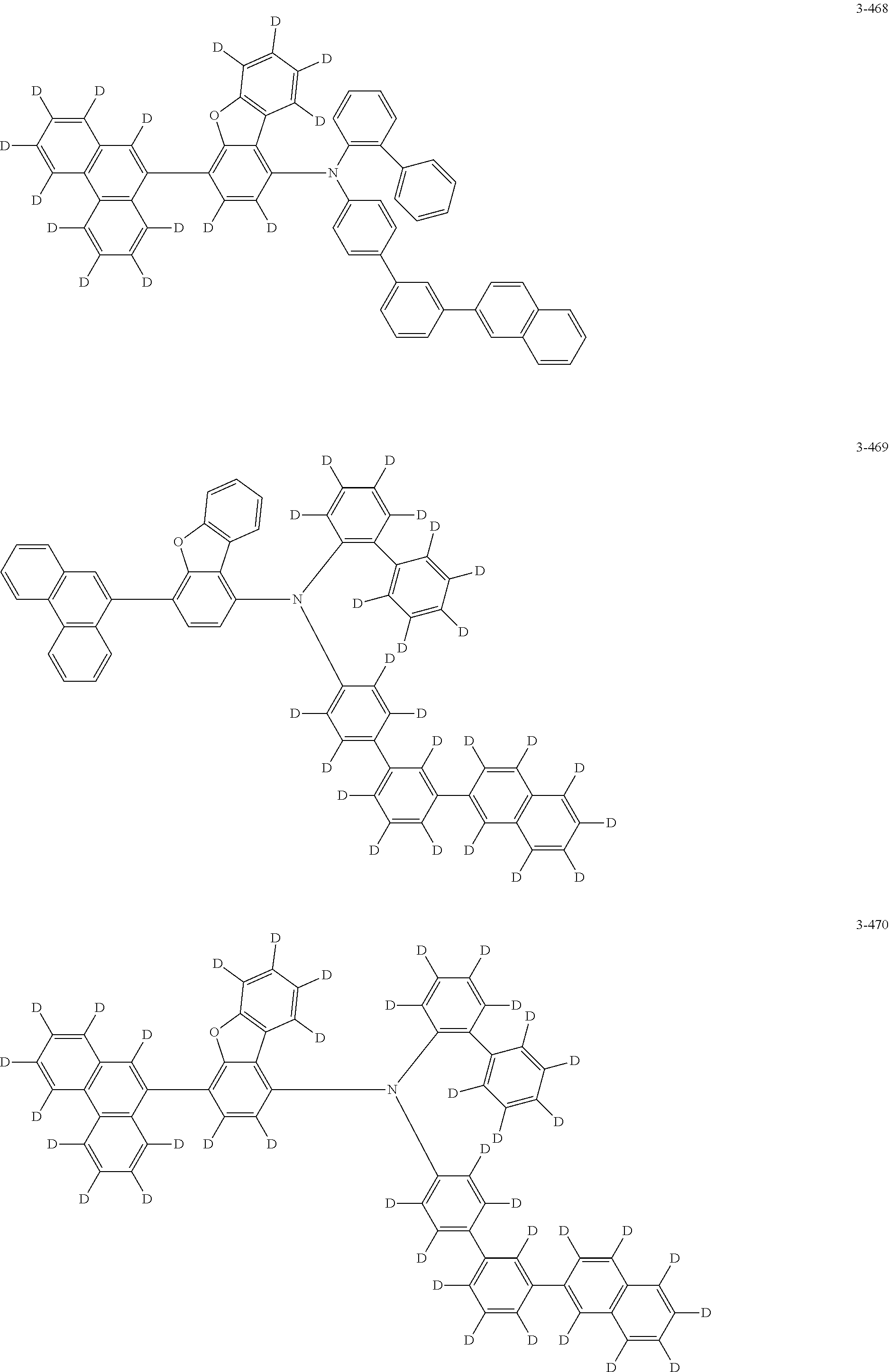

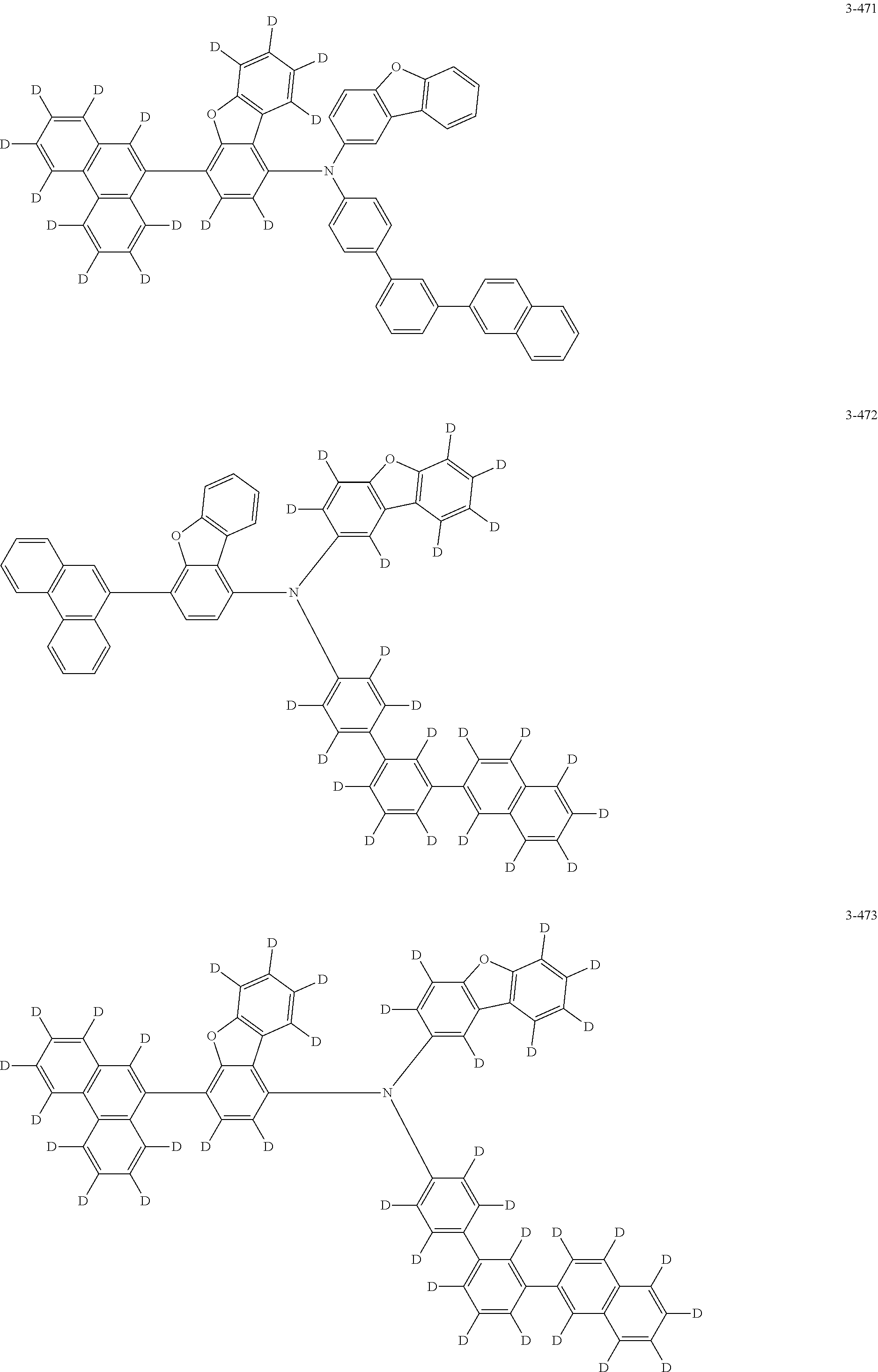

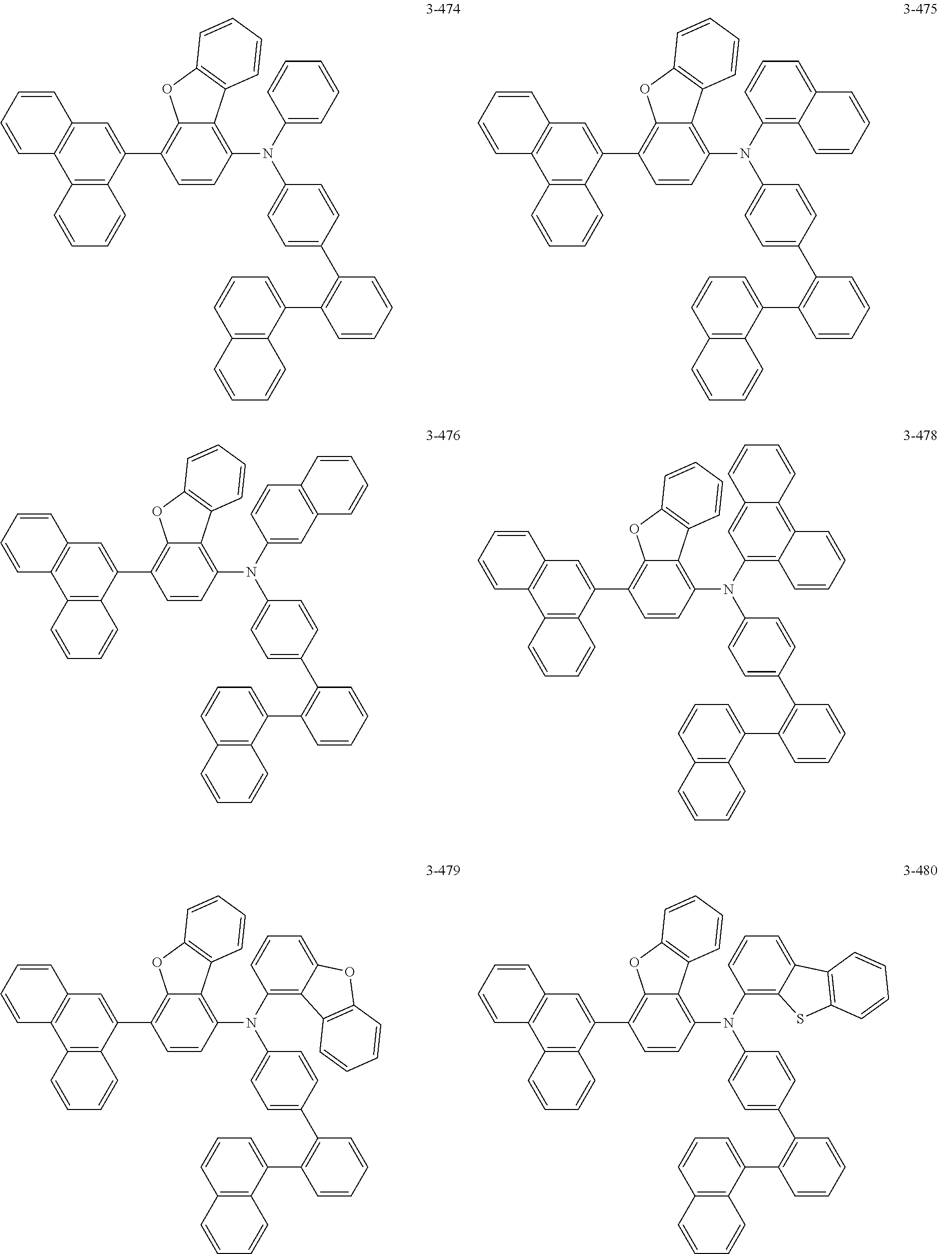

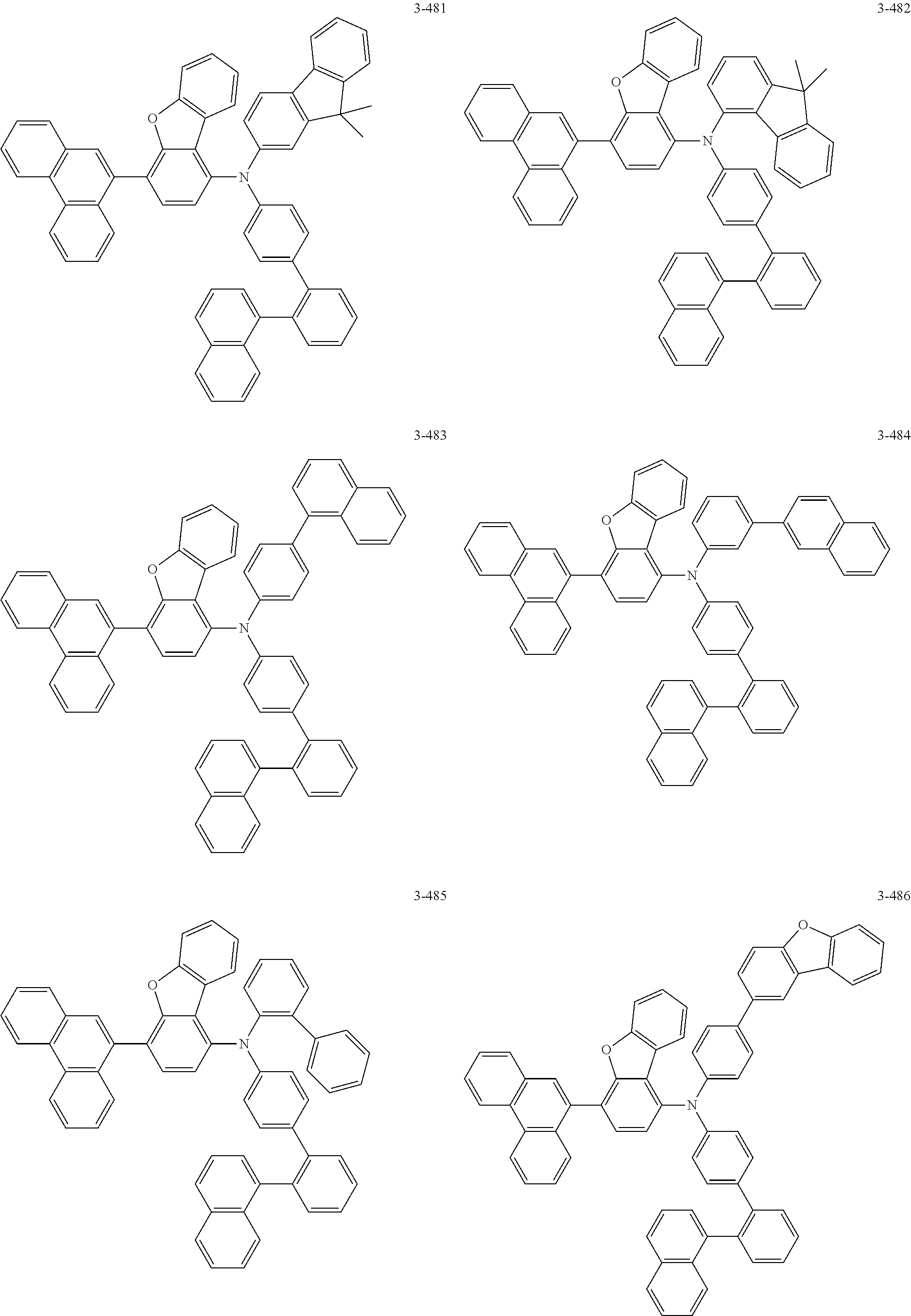

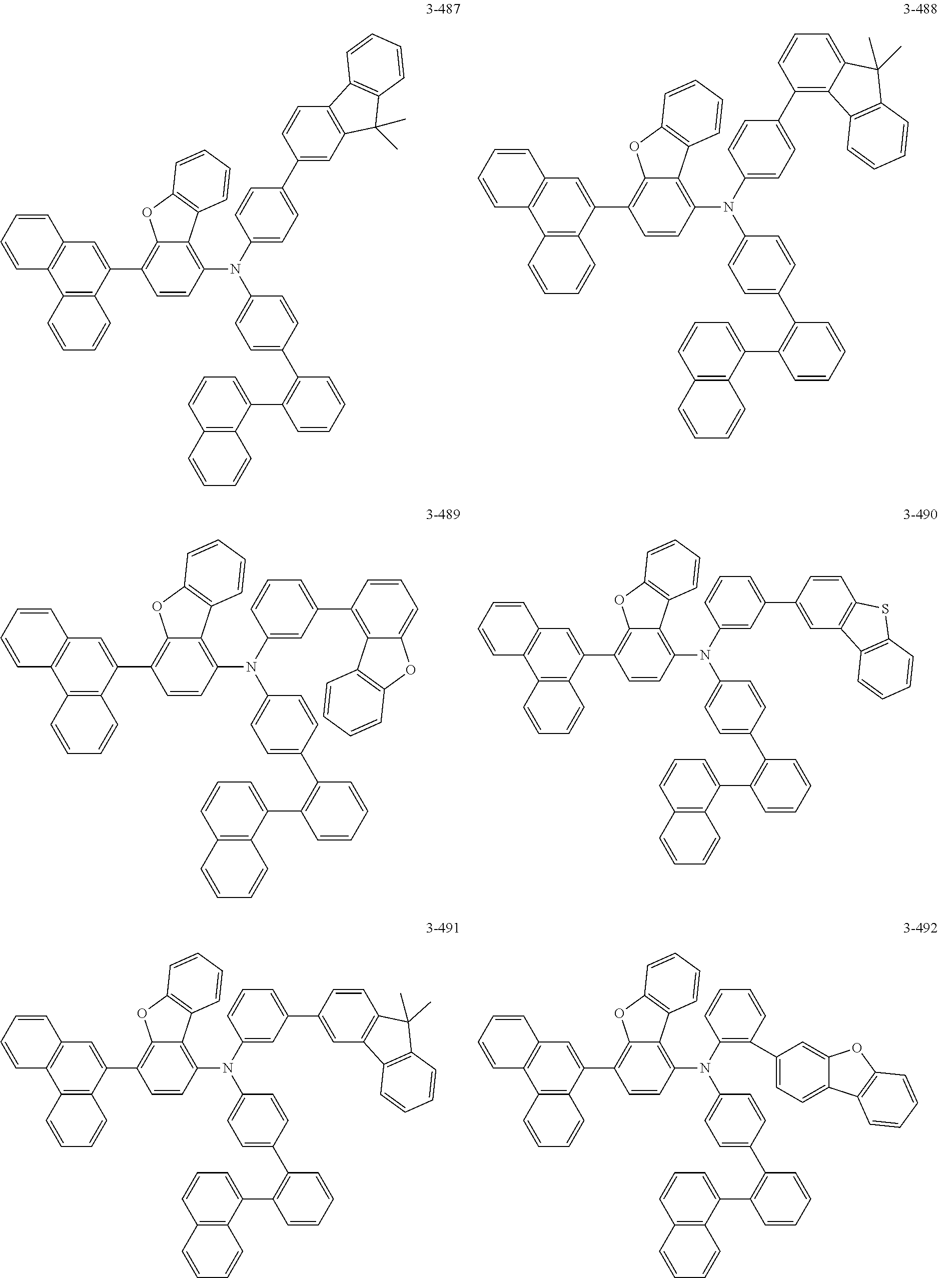

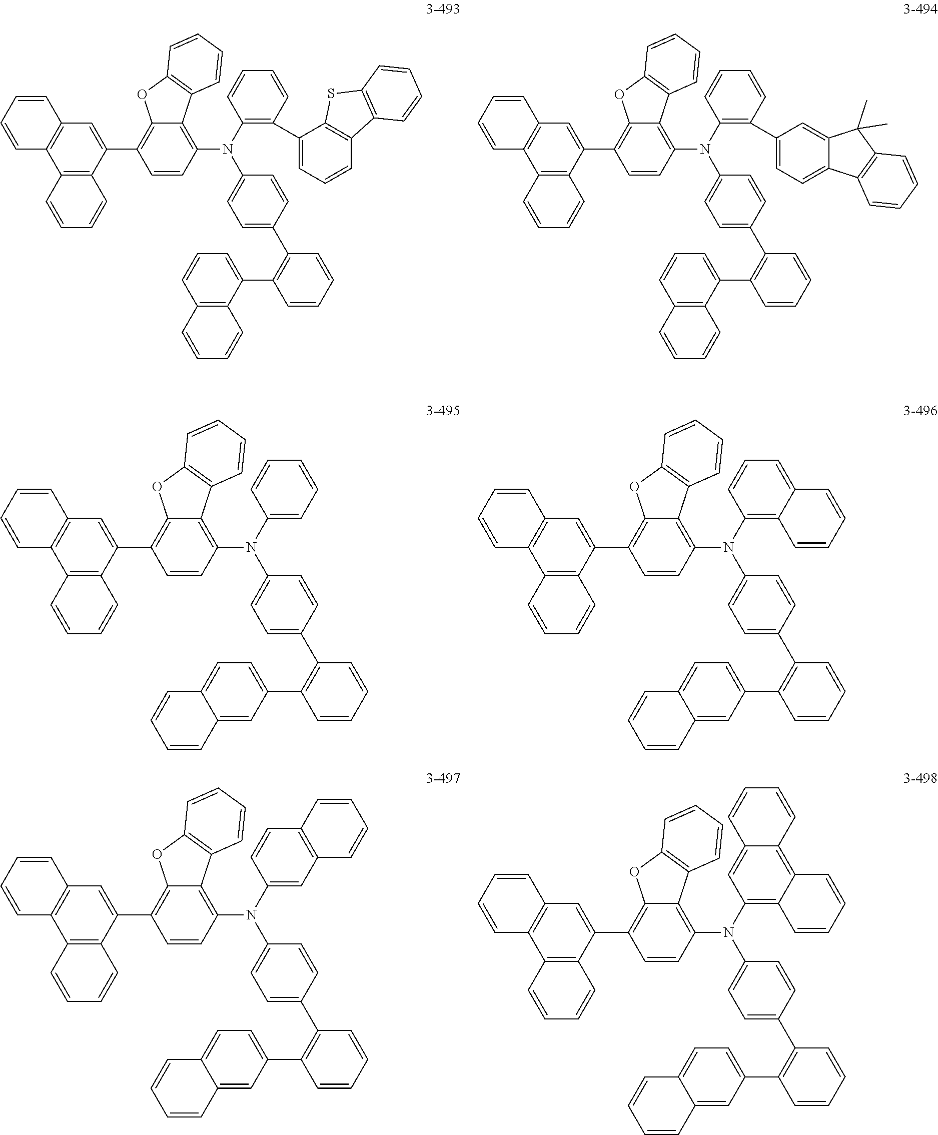

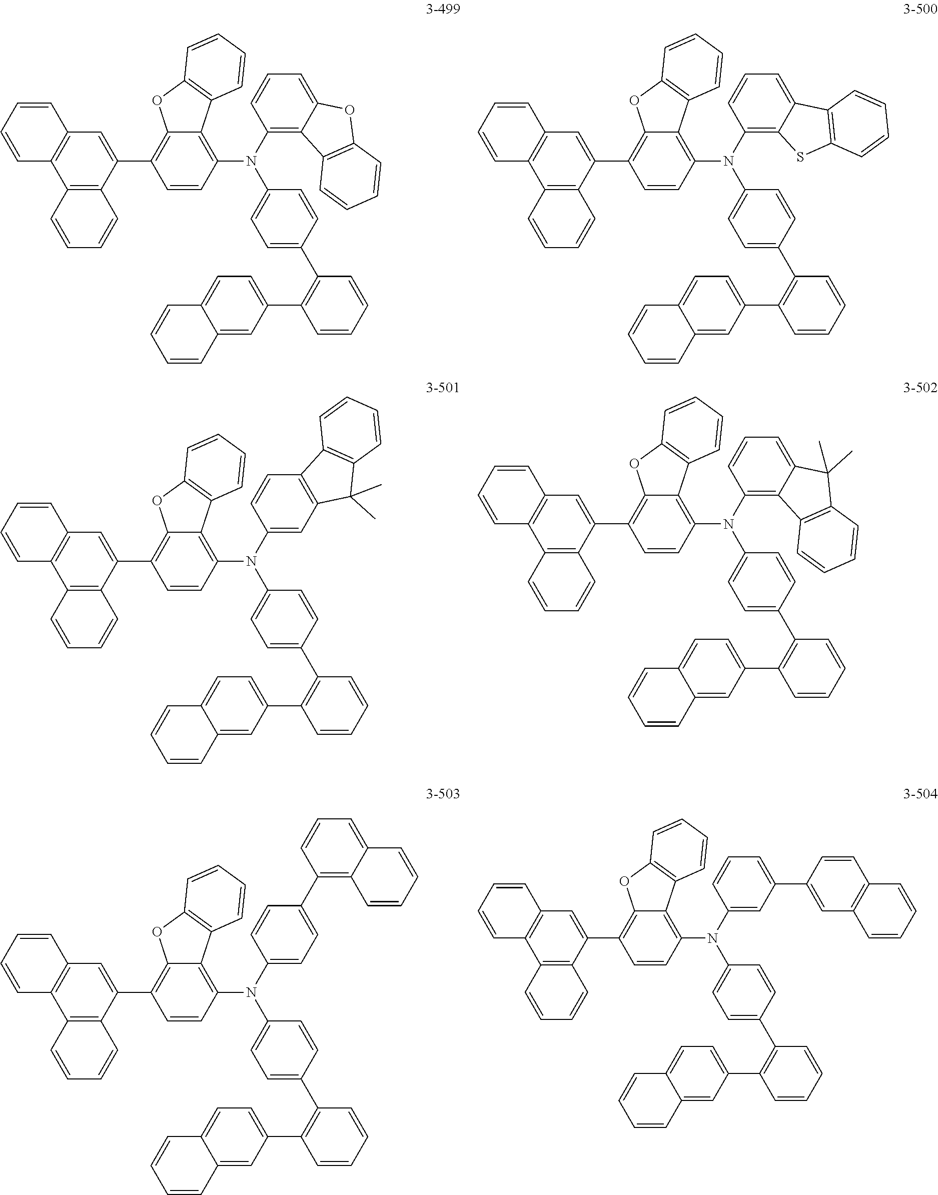

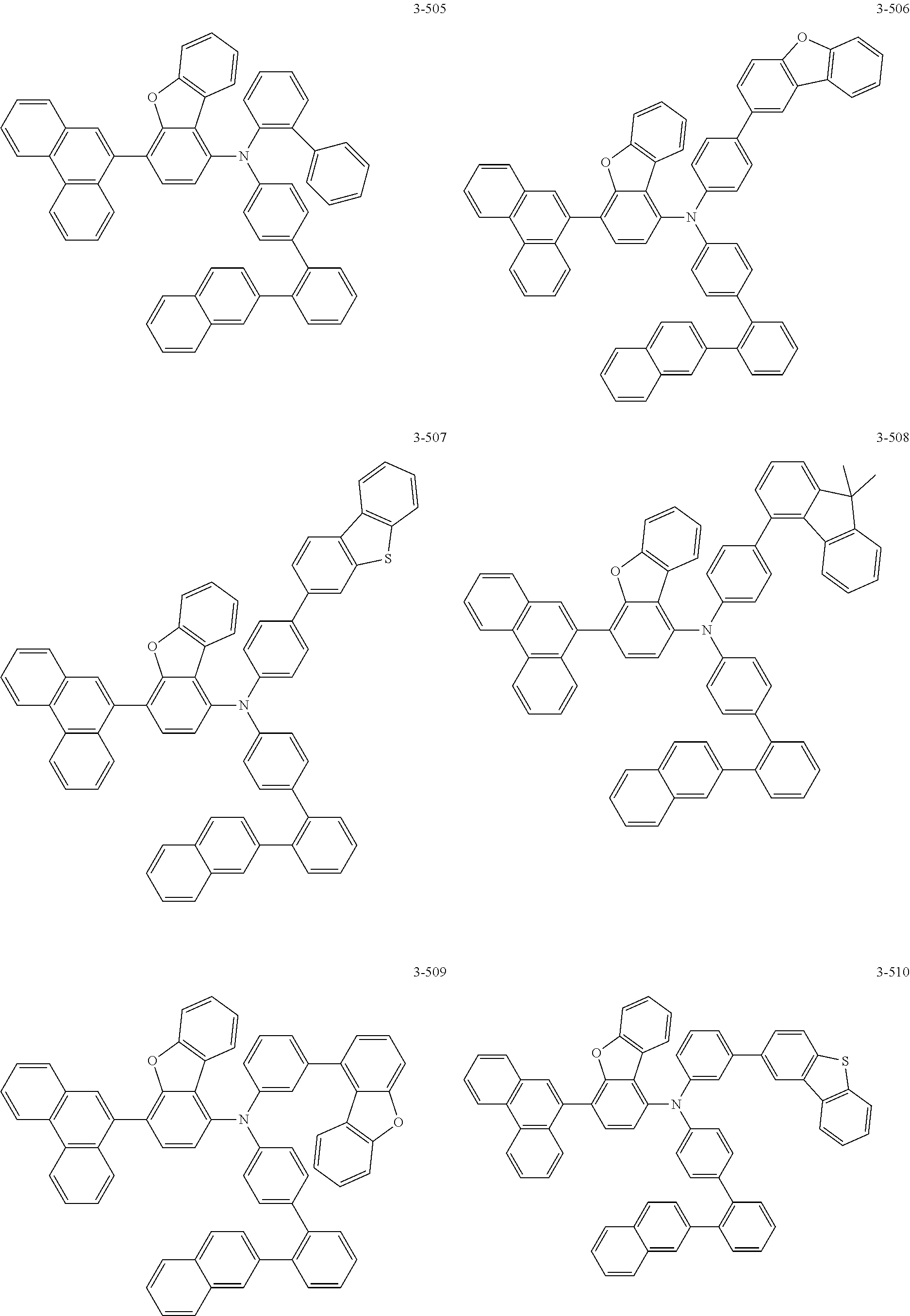

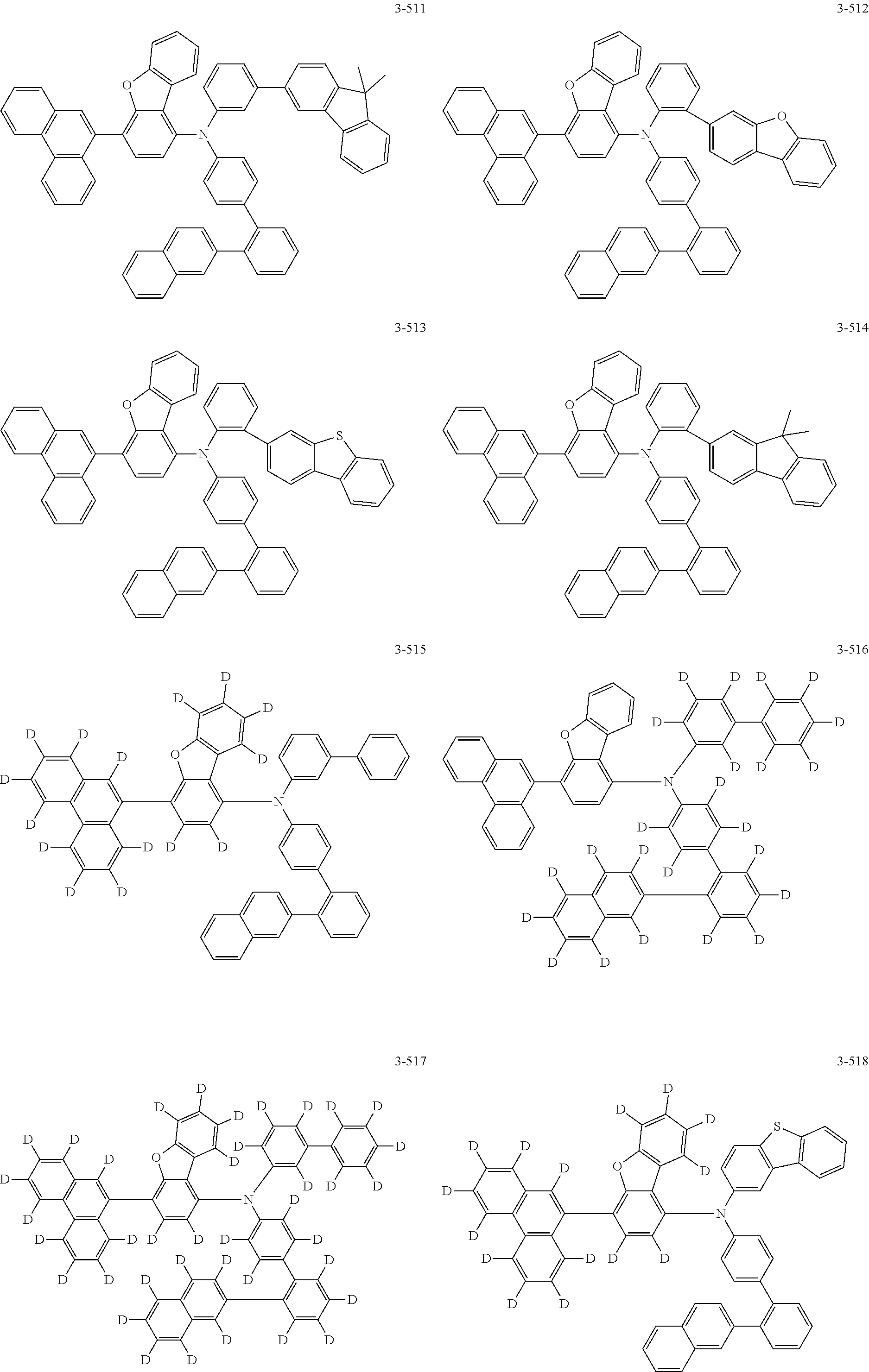

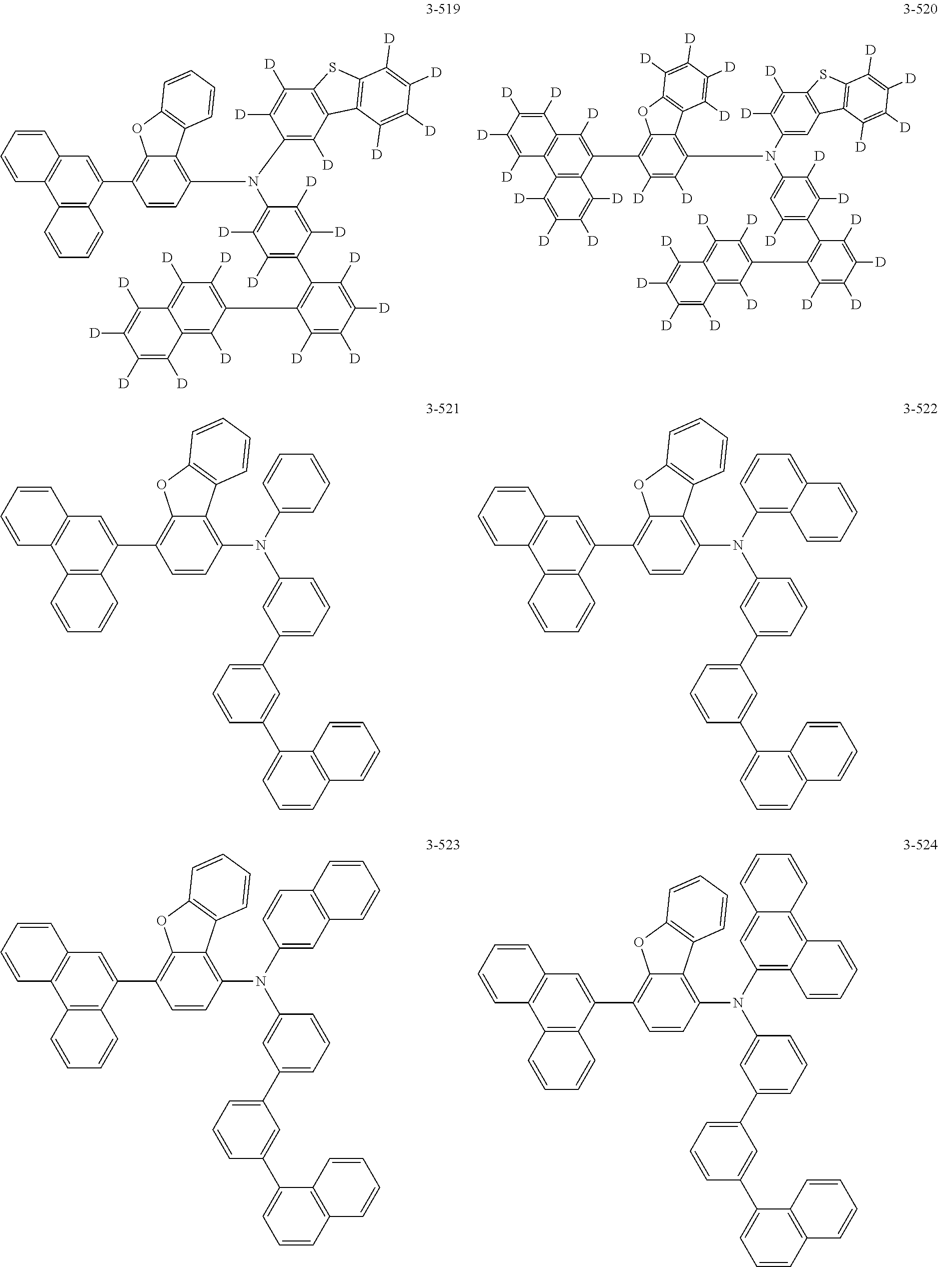

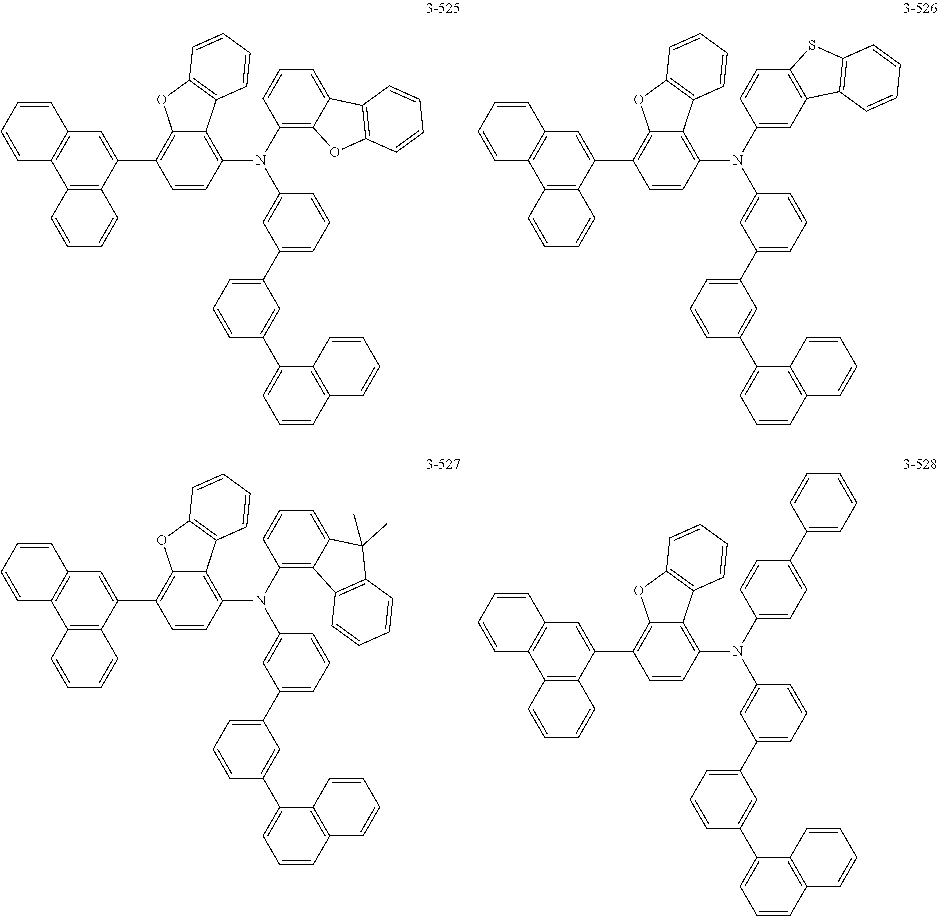 [SMILES: c1cc(-c2cccc(N(c3cccc4c3oc3ccccc34)c3ccc(-c4cc5ccccc5c5ccccc45)c4oc5ccccc5c34)c2)cc(-c2cccc3ccccc23)c1 ; c1cc(-c2cccc(N(c3ccc4sc5ccccc5c4c3)c3ccc(-c4cc5ccccc5c5ccccc45)c4oc5ccccc5c34)c2)cc(-c2cccc3ccccc23)c1 ; CC1(C)c2ccccc2-c2c(N(c3cccc(-c4cccc(-c5cccc6ccccc56)c4)c3)c3ccc(-c4cc5ccccc5c5ccccc45)c4oc5ccccc5c34)cccc21 ; c1ccc(-c2ccc(N(c3cccc(-c4cccc(-c5cccc6ccccc56)c4)c3)c3ccc(-c4cc5ccccc5c5ccccc45)c4oc5ccccc5c34)cc2)cc1]

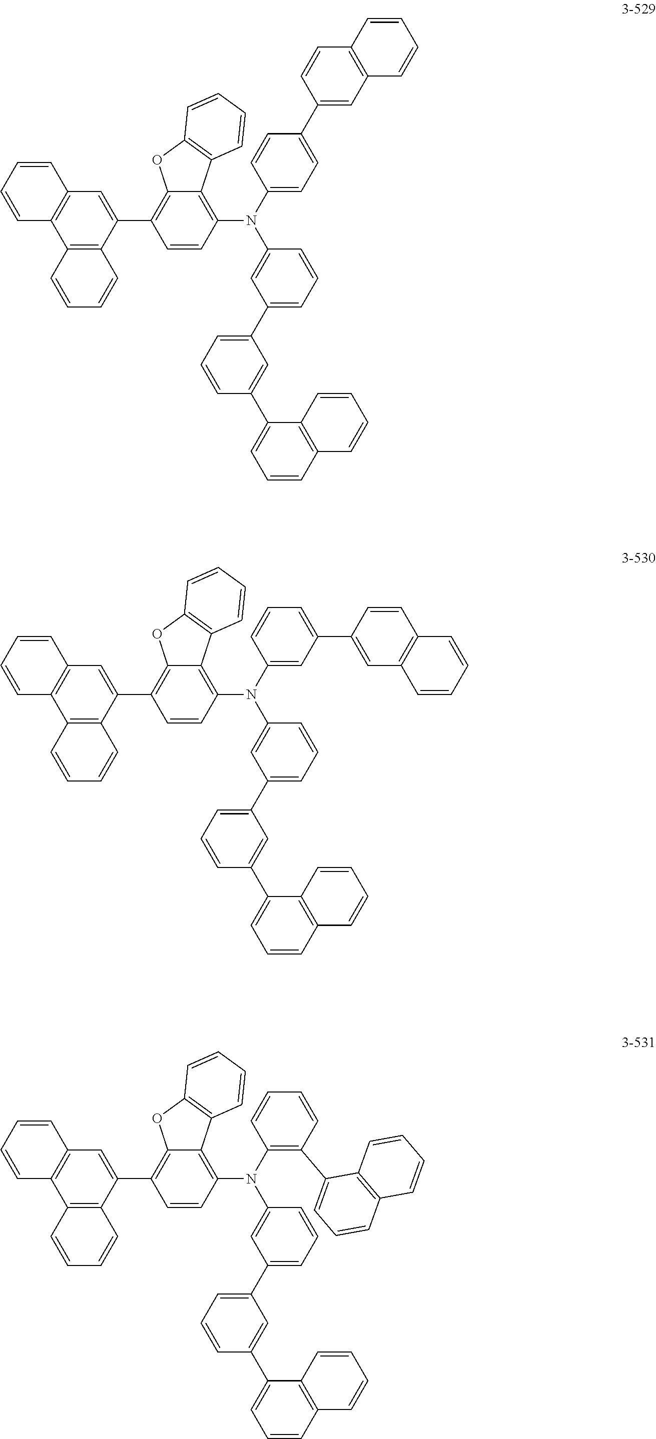 [SMILES: c1cc(-c2cccc(N(c3ccc(-c4ccc5ccccc5c4)cc3)c3ccc(-c4cc5ccccc5c5ccccc45)c4oc5ccccc5c34)c2)cc(-c2cccc3ccccc23)c1 ; c1cc(-c2cccc(N(c3cccc(-c4ccc5ccccc5c4)c3)c3ccc(-c4cc5ccccc5c5ccccc45)c4oc5ccccc5c34)c2)cc(-c2cccc3ccccc23)c1 ; c1cc(-c2cccc(N(c3ccccc3-c3cccc4ccccc34)c3ccc(-c4cc5ccccc5c5ccccc45)c4oc5ccccc5c34)c2)cc(-c2cccc3ccccc23)c1]

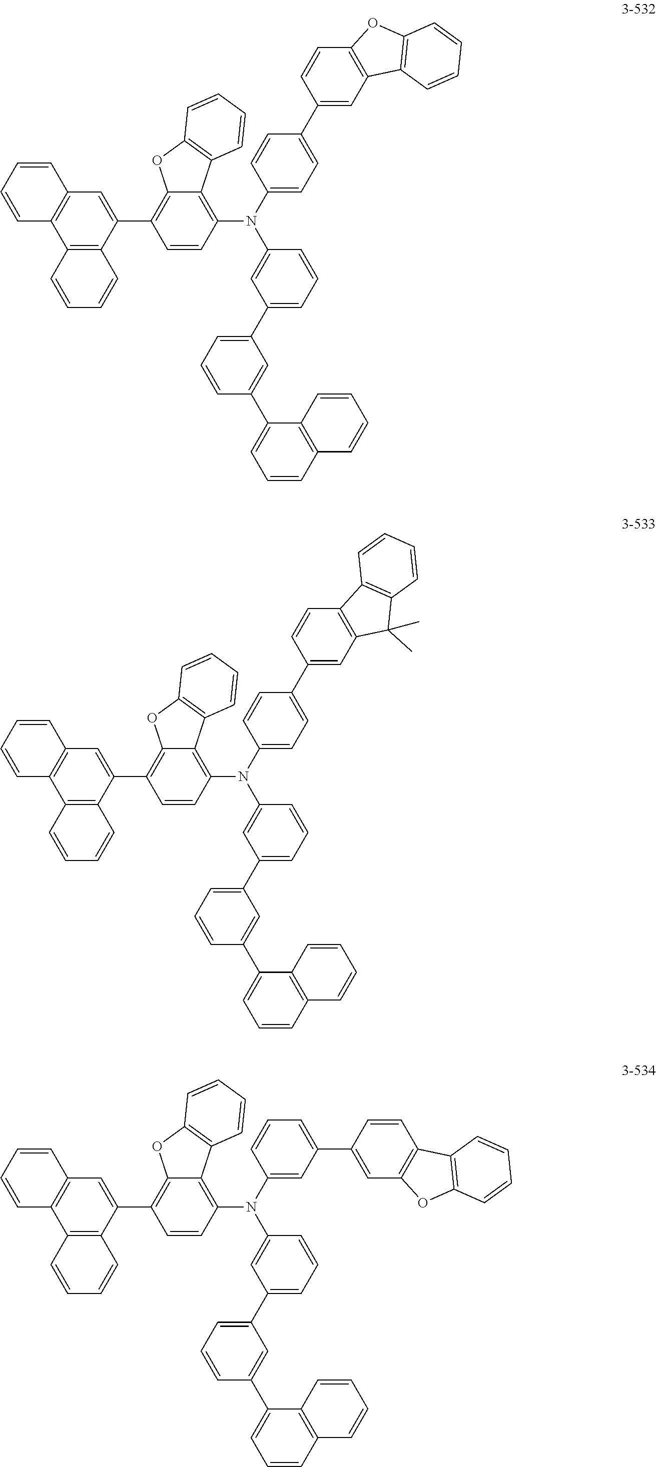 [SMILES: c1cc(-c2cccc(N(c3ccc(-c4ccc5oc6ccccc6c5c4)cc3)c3ccc(-c4cc5ccccc5c5ccccc45)c4oc5ccccc5c34)c2)cc(-c2cccc3ccccc23)c1 ; CC1(C)c2ccccc2-c2ccc(-c3ccc(N(c4cccc(-c5cccc(-c6cccc7ccccc67)c5)c4)c4ccc(-c5cc6ccccc6c6ccccc56)c5oc6ccccc6c45)cc3)cc21 ; c1cc(-c2cccc(N(c3cccc(-c4ccc5c(c4)oc4ccccc45)c3)c3ccc(-c4cc5ccccc5c5ccccc45)c4oc5ccccc5c34)c2)cc(-c2cccc3ccccc23)c1]

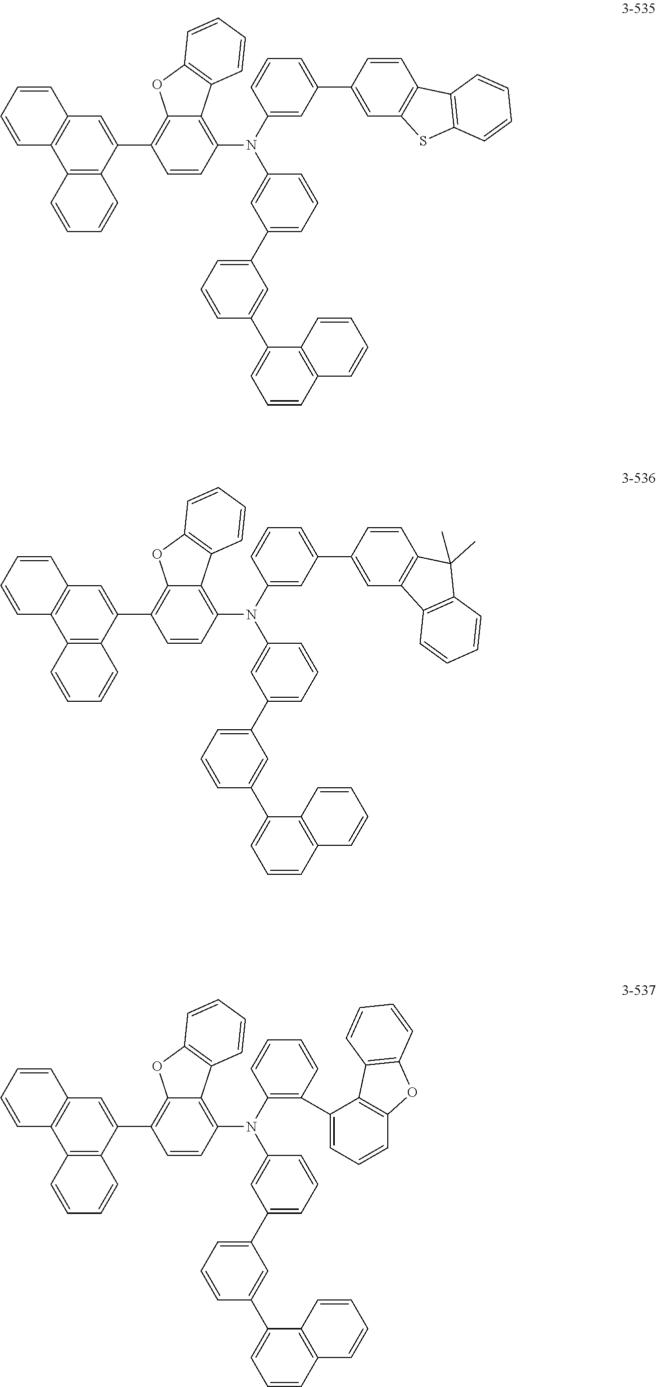 [SMILES: c1cc(-c2cccc(N(c3cccc(-c4ccc5c(c4)sc4ccccc45)c3)c3ccc(-c4cc5ccccc5c5ccccc45)c4oc5ccccc5c34)c2)cc(-c2cccc3ccccc23)c1 ; CC1(C)c2ccccc2-c2cc(-c3cccc(N(c4cccc(-c5cccc(-c6cccc7ccccc67)c5)c4)c4ccc(-c5cc6ccccc6c6ccccc56)c5oc6ccccc6c45)c3)ccc21 ; c1cc(-c2cccc(N(c3ccccc3-c3cccc4oc5ccccc5c34)c3ccc(-c4cc5ccccc5c5ccccc45)c4oc5ccccc5c34)c2)cc(-c2cccc3ccccc23)c1]

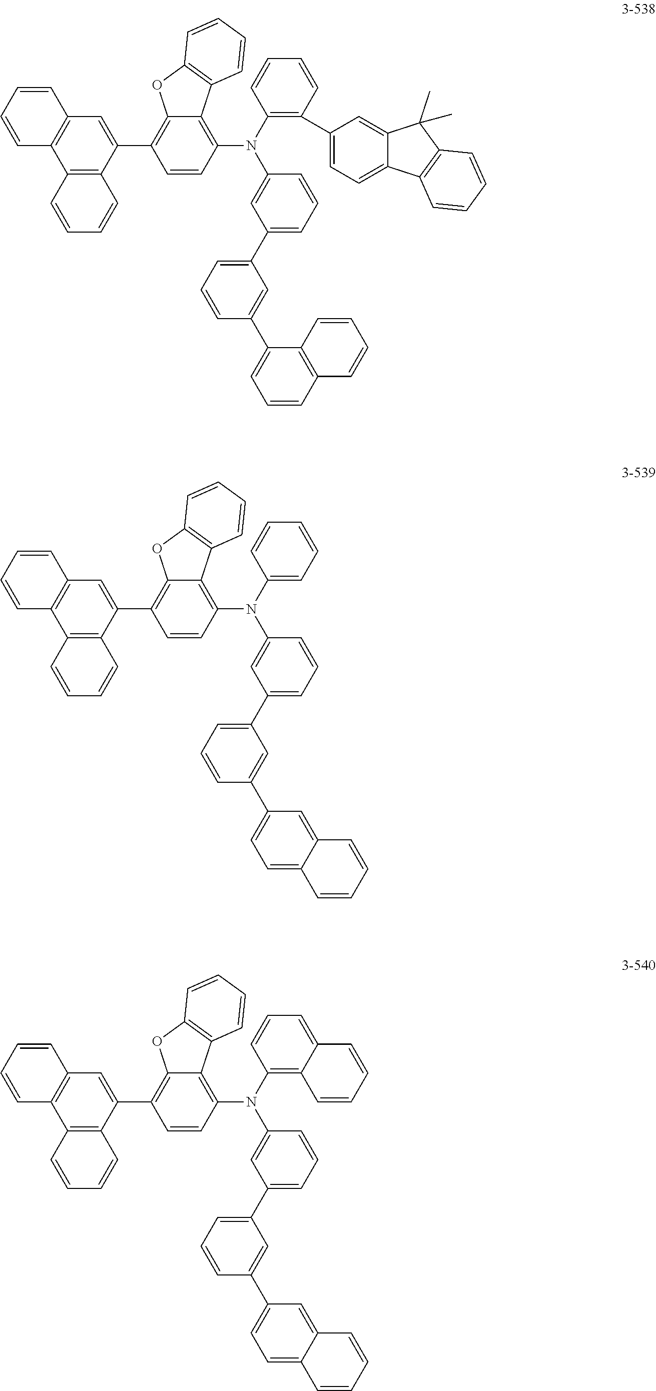 [SMILES: CC1(C)c2ccccc2-c2ccc(-c3ccccc3N(c3cccc(-c4cccc(-c5cccc6ccccc56)c4)c3)c3ccc(-c4cc5ccccc5c5ccccc45)c4oc5ccccc5c34)cc21 ; c1ccc(N(c2cccc(-c3cccc(-c4ccc5ccccc5c4)c3)c2)c2ccc(-c3cc4ccccc4c4ccccc34)c3oc4ccccc4c23)cc1 ; c1cc(-c2cccc(N(c3cccc4ccccc34)c3ccc(-c4cc5ccccc5c5ccccc45)c4oc5ccccc5c34)c2)cc(-c2ccc3ccccc3c2)c1]

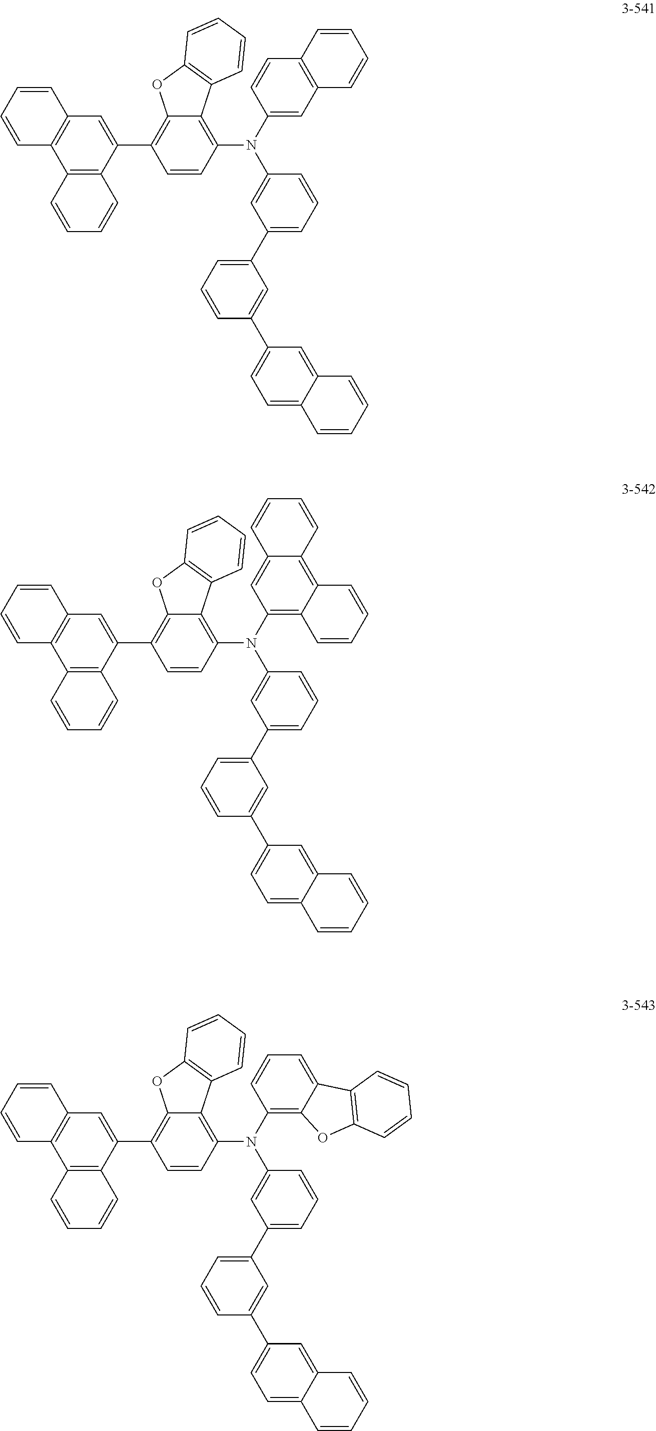 [SMILES: c1cc(-c2cccc(N(c3ccc4ccccc4c3)c3ccc(-c4cc5ccccc5c5ccccc45)c4oc5ccccc5c34)c2)cc(-c2ccc3ccccc3c2)c1 ; c1cc(-c2cccc(N(c3cc4ccccc4c4ccccc34)c3ccc(-c4cc5ccccc5c5ccccc45)c4oc5ccccc5c34)c2)cc(-c2ccc3ccccc3c2)c1 ; c1cc(-c2cccc(N(c3cccc4c3oc3ccccc34)c3ccc(-c4cc5ccccc5c5ccccc45)c4oc5ccccc5c34)c2)cc(-c2ccc3ccccc3c2)c1]

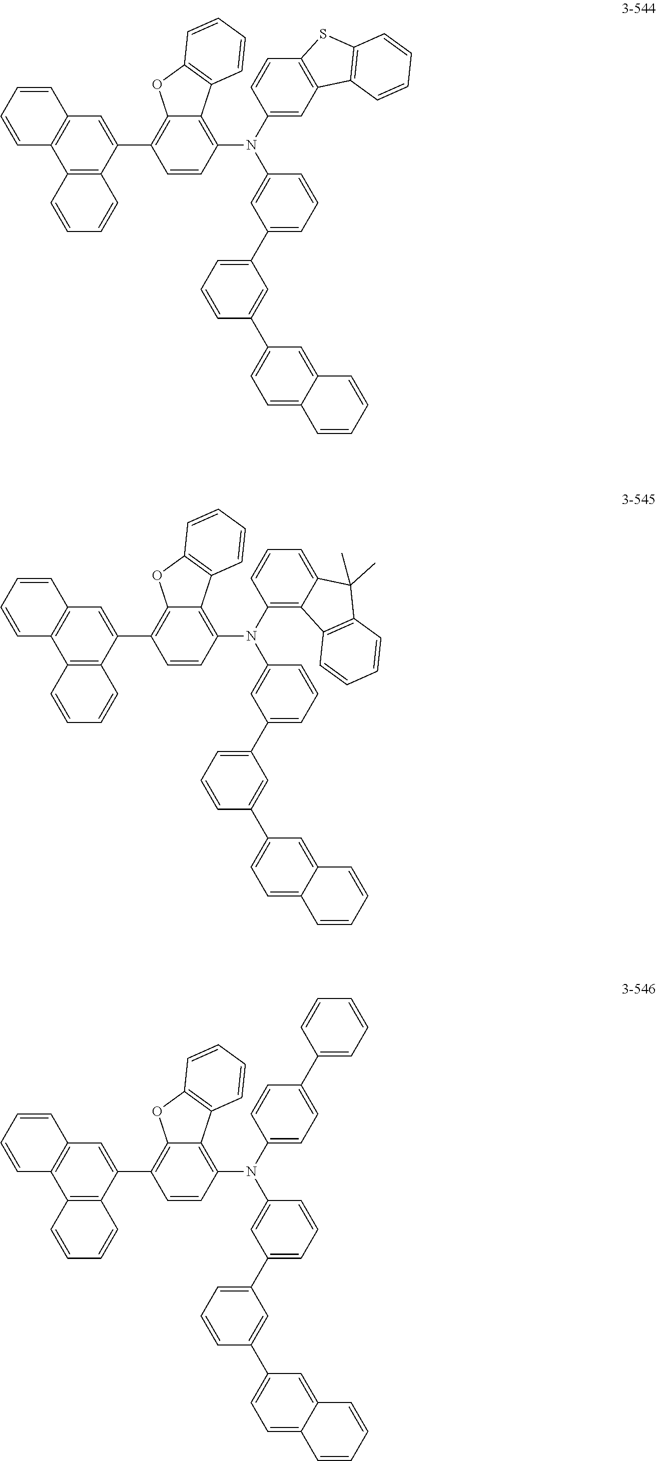 [SMILES: c1cc(-c2cccc(N(c3ccc4sc5ccccc5c4c3)c3ccc(-c4cc5ccccc5c5ccccc45)c4oc5ccccc5c34)c2)cc(-c2ccc3ccccc3c2)c1 ; CC1(C)c2ccccc2-c2c(N(c3cccc(-c4cccc(-c5ccc6ccccc6c5)c4)c3)c3ccc(-c4cc5ccccc5c5ccccc45)c4oc5ccccc5c34)cccc21 ; c1ccc(-c2ccc(N(c3cccc(-c4cccc(-c5ccc6ccccc6c5)c4)c3)c3ccc(-c4cc5ccccc5c5ccccc45)c4oc5ccccc5c34)cc2)cc1]

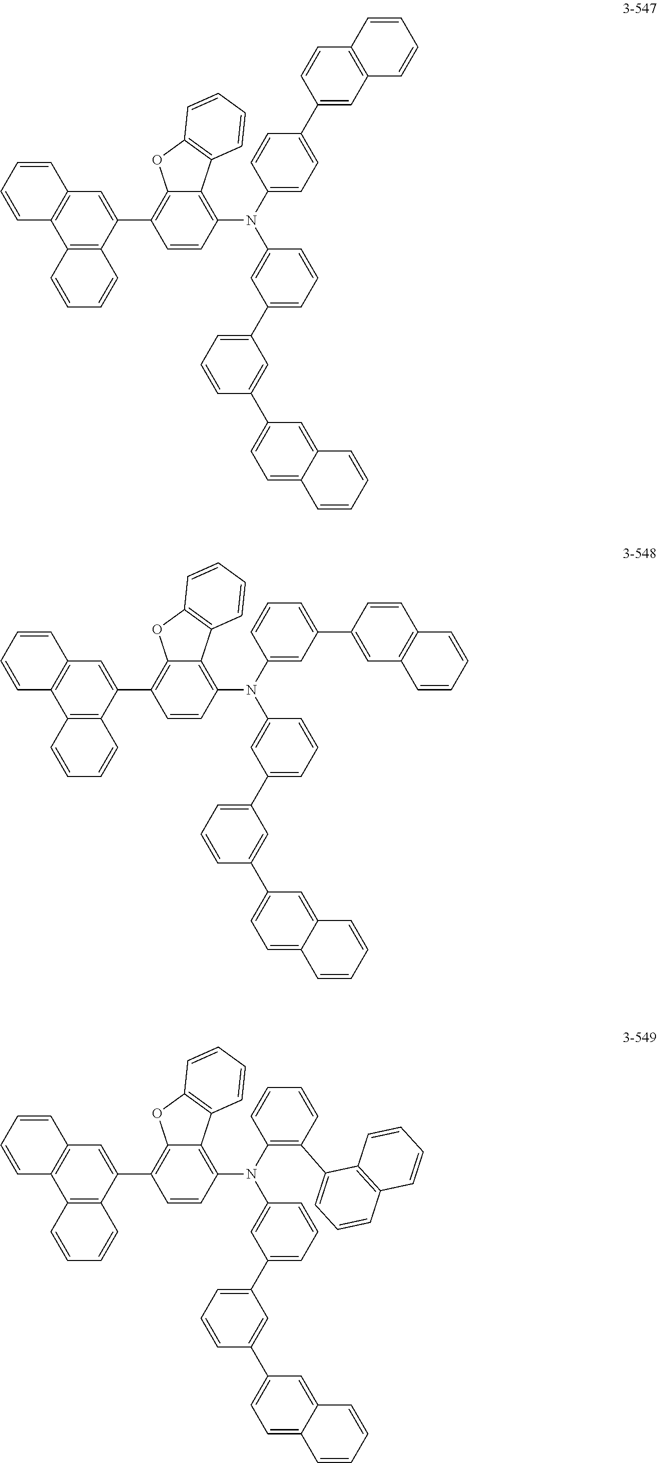 [SMILES: c1cc(-c2cccc(N(c3ccc(-c4ccc5ccccc5c4)cc3)c3ccc(-c4cc5ccccc5c5ccccc45)c4oc5ccccc5c34)c2)cc(-c2ccc3ccccc3c2)c1 ; c1cc(-c2cccc(N(c3cccc(-c4ccc5ccccc5c4)c3)c3ccc(-c4cc5ccccc5c5ccccc45)c4oc5ccccc5c34)c2)cc(-c2ccc3ccccc3c2)c1 ; c1cc(-c2cccc(N(c3ccccc3-c3cccc4ccccc34)c3ccc(-c4cc5ccccc5c5ccccc45)c4oc5ccccc5c34)c2)cc(-c2ccc3ccccc3c2)c1]

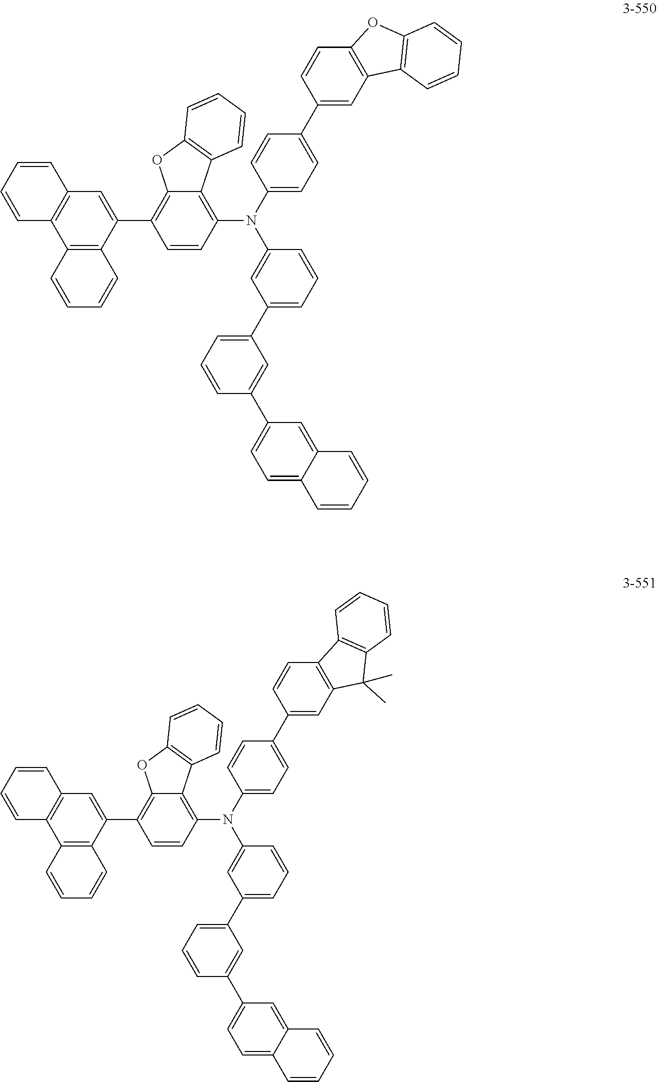 [SMILES: c1cc(-c2cccc(N(c3ccc(-c4ccc5oc6ccccc6c5c4)cc3)c3ccc(-c4cc5ccccc5c5ccccc45)c4oc5ccccc5c34)c2)cc(-c2ccc3ccccc3c2)c1 ; CC1(C)c2ccccc2-c2ccc(-c3ccc(N(c4cccc(-c5cccc(-c6ccc7ccccc7c6)c5)c4)c4ccc(-c5cc6ccccc6c6ccccc56)c5oc6ccccc6c45)cc3)cc21]

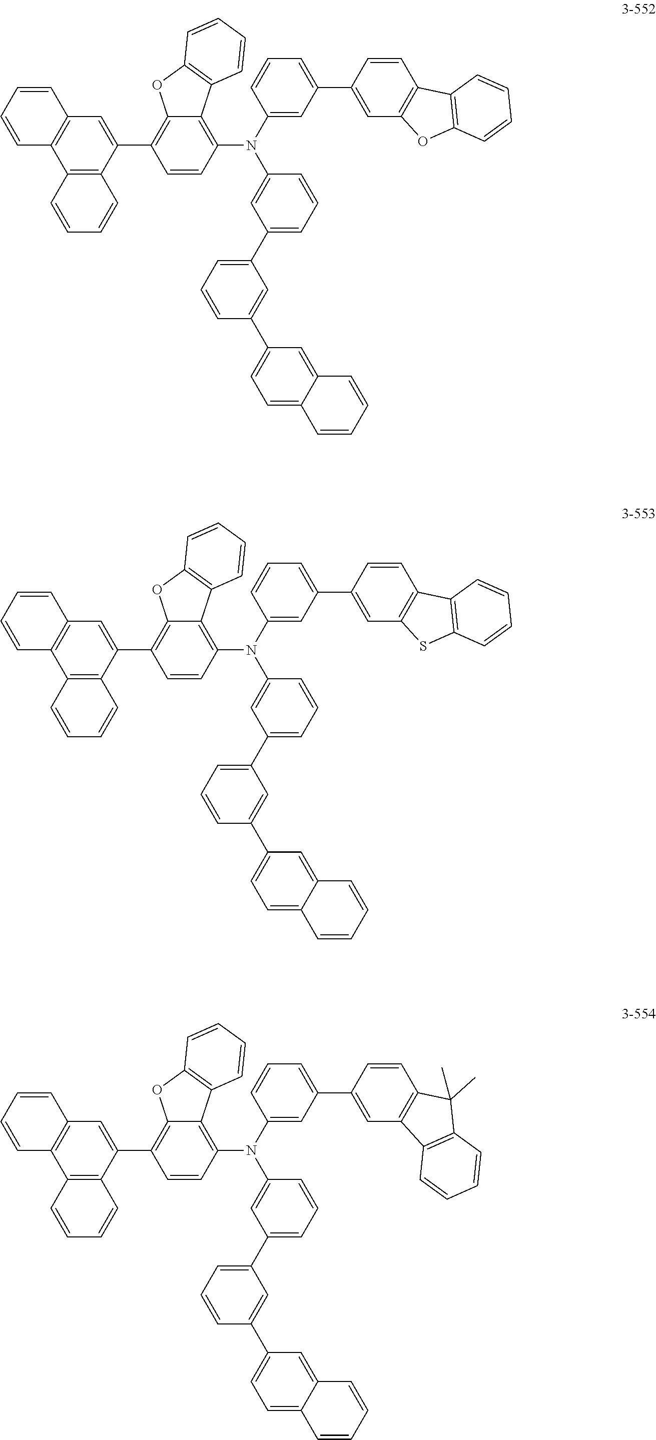 [SMILES: c1cc(-c2cccc(N(c3cccc(-c4ccc5c(c4)oc4ccccc45)c3)c3ccc(-c4cc5ccccc5c5ccccc45)c4oc5ccccc5c34)c2)cc(-c2ccc3ccccc3c2)c1 ; c1cc(-c2cccc(N(c3cccc(-c4ccc5c(c4)sc4ccccc45)c3)c3ccc(-c4cc5ccccc5c5ccccc45)c4oc5ccccc5c34)c2)cc(-c2ccc3ccccc3c2)c1 ; CC1(C)c2ccccc2-c2cc(-c3cccc(N(c4cccc(-c5cccc(-c6ccc7ccccc7c6)c5)c4)c4ccc(-c5cc6ccccc6c6ccccc56)c5oc6ccccc6c45)c3)ccc21]

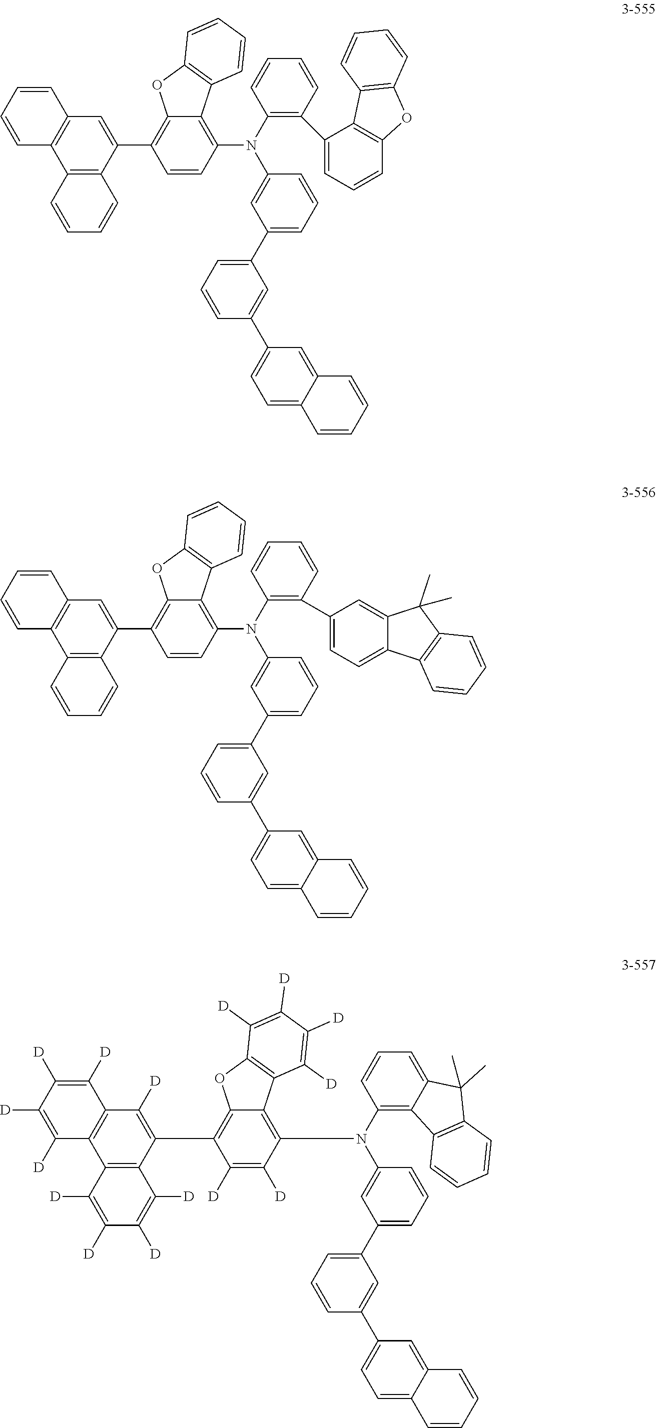 [SMILES: c1cc(-c2cccc(N(c3ccccc3-c3cccc4oc5ccccc5c34)c3ccc(-c4cc5ccccc5c5ccccc45)c4oc5ccccc5c34)c2)cc(-c2ccc3ccccc3c2)c1 ; CC1(C)c2ccccc2-c2ccc(-c3ccccc3N(c3cccc(-c4cccc(-c5ccc6ccccc6c5)c4)c3)c3ccc(-c4cc5ccccc5c5ccccc45)c4oc5ccccc5c34)cc21 ; [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(N(c4cccc(-c5cccc(-c6ccc7ccccc7c6)c5)c4)c4cccc5c4-c4ccccc4C5(C)C)c32)c1[2H]]

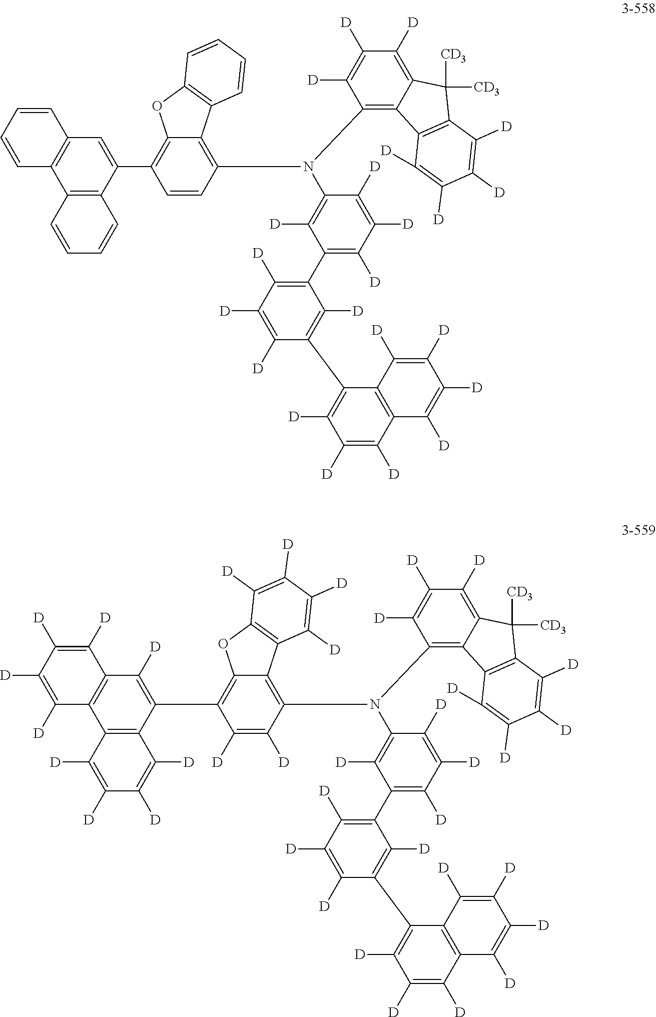

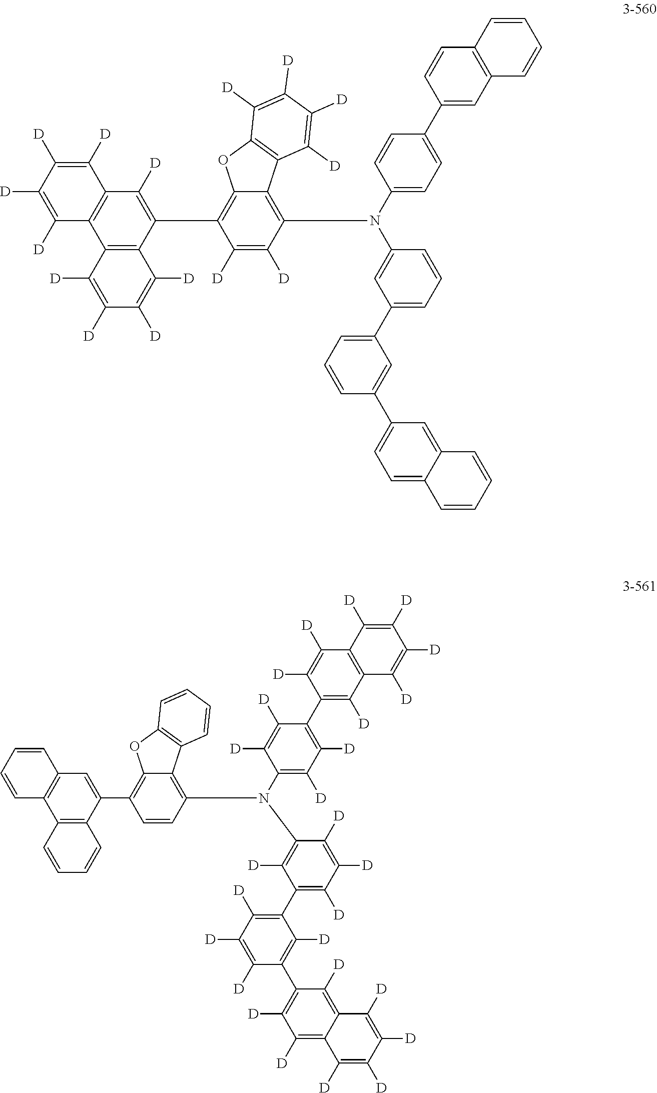 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(N(c4ccc(-c5ccc6ccccc6c5)cc4)c4cccc(-c5cccc(-c6ccc7ccccc7c6)c5)c4)c32)c1[2H] ; [2H]c1c([2H])c(-c2c([2H])c([2H])c([2H])c(N(c3c([2H])c([2H])c(-c4c([2H])c([2H])c5c([2H])c([2H])c([2H])c([2H])c5c4[2H])c([2H])c3[2H])c3ccc(-c4cc5ccccc5c5ccccc45)c4oc5ccccc5c34)c2[2H])c([2H])c(-c2c([2H])c([2H])c3c([2H])c([2H])c([2H])c([2H])c3c2[2H])c1[2H]]

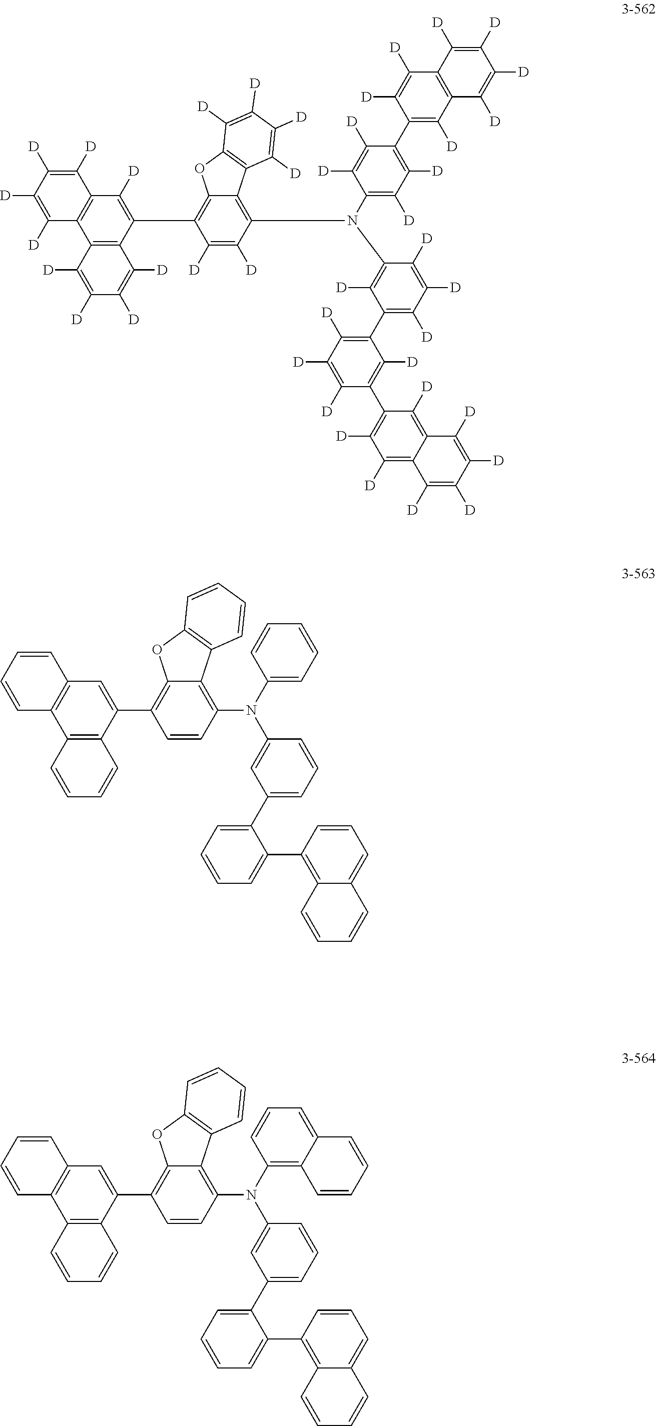

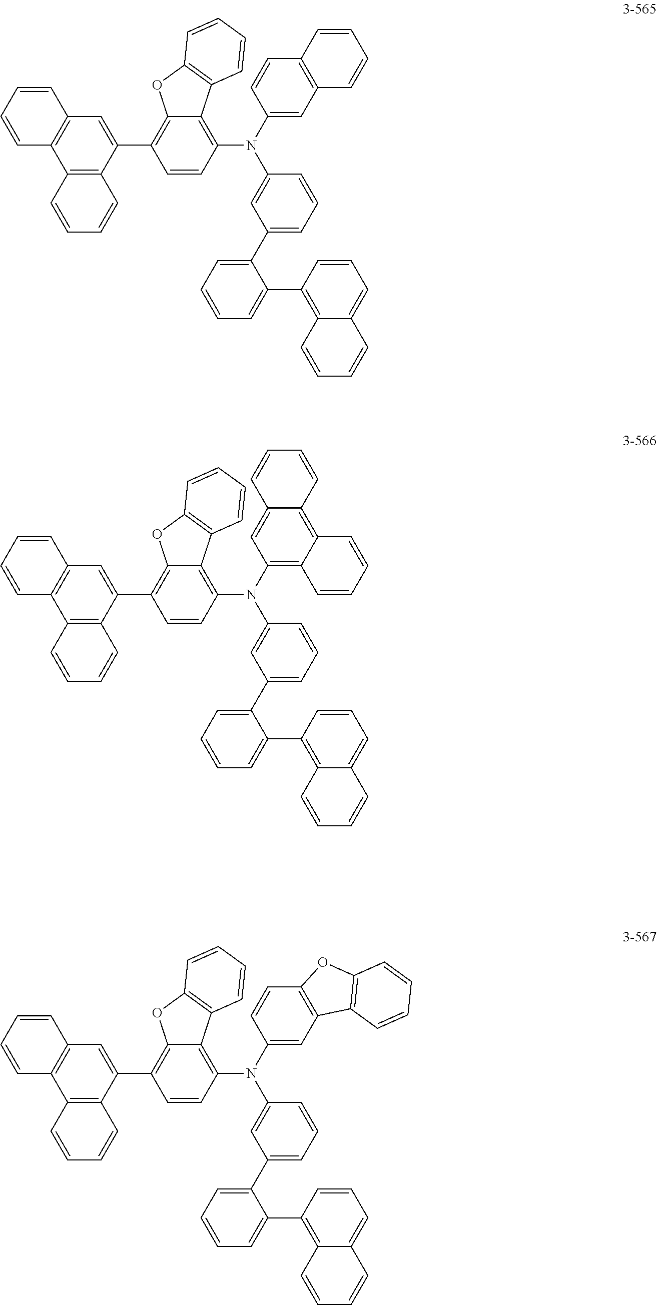 [SMILES: c1cc(-c2ccccc2-c2cccc3ccccc23)cc(N(c2ccc3ccccc3c2)c2ccc(-c3cc4ccccc4c4ccccc34)c3oc4ccccc4c23)c1 ; c1cc(-c2ccccc2-c2cccc3ccccc23)cc(N(c2cc3ccccc3c3ccccc23)c2ccc(-c3cc4ccccc4c4ccccc34)c3oc4ccccc4c23)c1 ; c1cc(-c2ccccc2-c2cccc3ccccc23)cc(N(c2ccc3oc4ccccc4c3c2)c2ccc(-c3cc4ccccc4c4ccccc34)c3oc4ccccc4c23)c1]

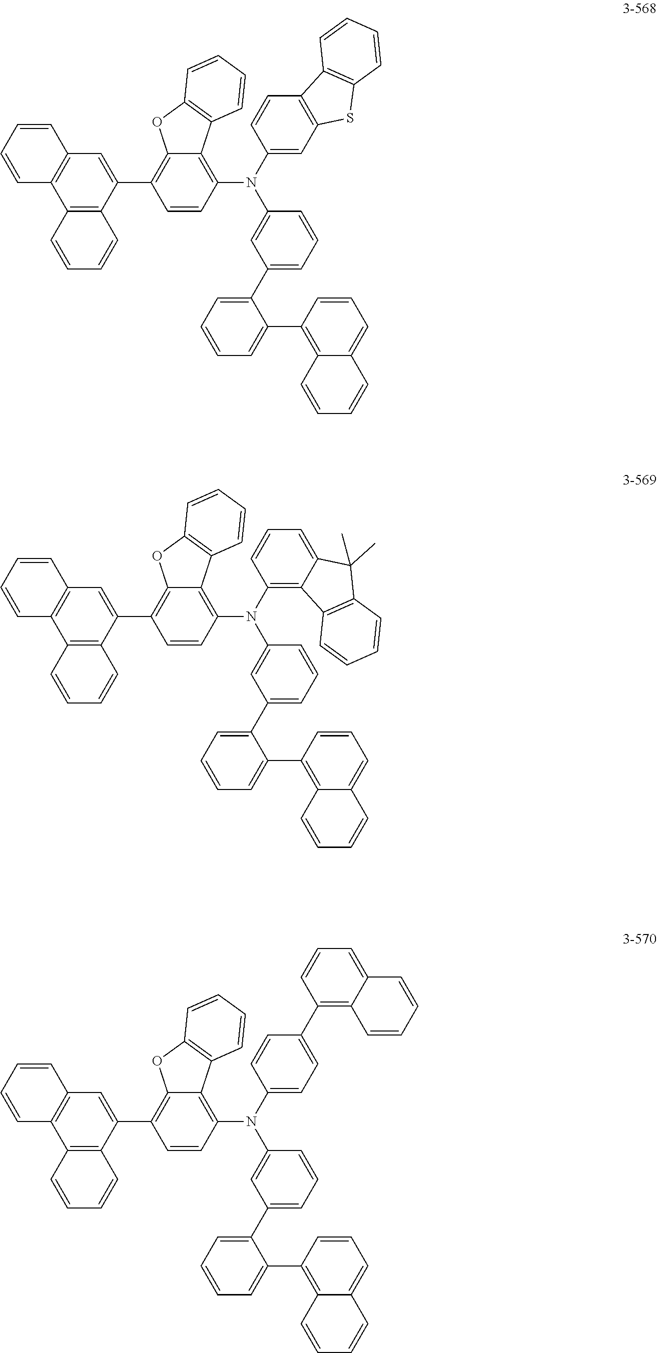 [SMILES: c1cc(-c2ccccc2-c2cccc3ccccc23)cc(N(c2ccc3c(c2)sc2ccccc23)c2ccc(-c3cc4ccccc4c4ccccc34)c3oc4ccccc4c23)c1 ; CC1(C)c2ccccc2-c2c(N(c3cccc(-c4ccccc4-c4cccc5ccccc45)c3)c3ccc(-c4cc5ccccc5c5ccccc45)c4oc5ccccc5c34)cccc21 ; c1cc(-c2ccccc2-c2cccc3ccccc23)cc(N(c2ccc(-c3cccc4ccccc34)cc2)c2ccc(-c3cc4ccccc4c4ccccc34)c3oc4ccccc4c23)c1]

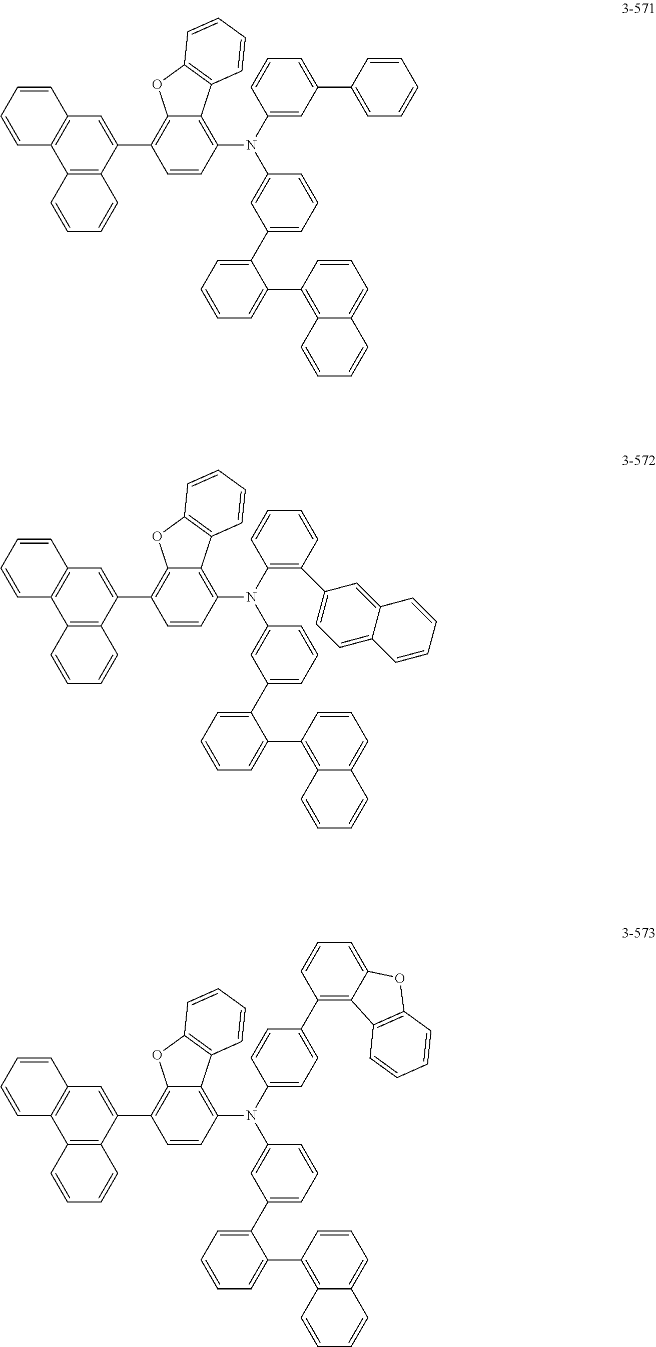 [SMILES: c1ccc(-c2cccc(N(c3cccc(-c4ccccc4-c4cccc5ccccc45)c3)c3ccc(-c4cc5ccccc5c5ccccc45)c4oc5ccccc5c34)c2)cc1 ; c1cc(-c2ccccc2-c2cccc3ccccc23)cc(N(c2ccccc2-c2ccc3ccccc3c2)c2ccc(-c3cc4ccccc4c4ccccc34)c3oc4ccccc4c23)c1 ; c1cc(-c2ccccc2-c2cccc3ccccc23)cc(N(c2ccc(-c3cccc4oc5ccccc5c34)cc2)c2ccc(-c3cc4ccccc4c4ccccc34)c3oc4ccccc4c23)c1]

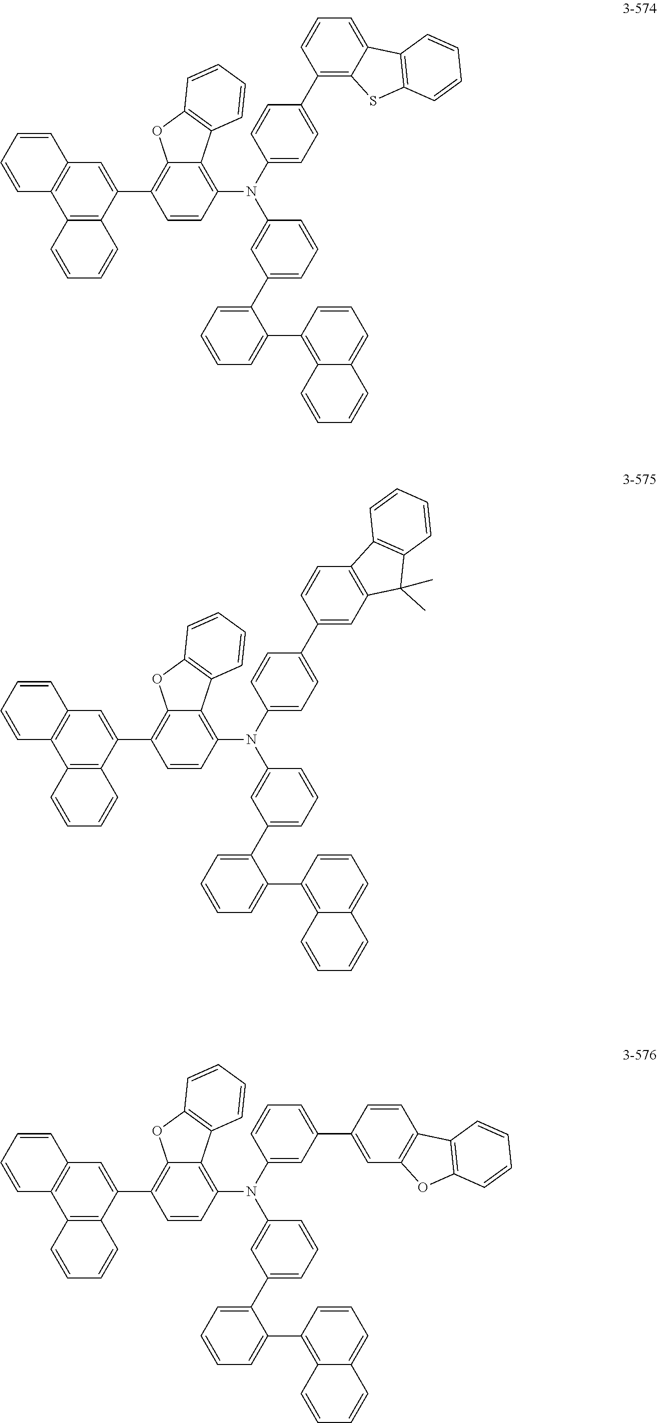 [SMILES: c1cc(-c2ccccc2-c2cccc3ccccc23)cc(N(c2ccc(-c3cccc4c3sc3ccccc34)cc2)c2ccc(-c3cc4ccccc4c4ccccc34)c3oc4ccccc4c23)c1 ; CC1(C)c2ccccc2-c2ccc(-c3ccc(N(c4cccc(-c5ccccc5-c5cccc6ccccc56)c4)c4ccc(-c5cc6ccccc6c6ccccc56)c5oc6ccccc6c45)cc3)cc21 ; c1cc(-c2ccc3c(c2)oc2ccccc23)cc(N(c2cccc(-c3ccccc3-c3cccc4ccccc34)c2)c2ccc(-c3cc4ccccc4c4ccccc34)c3oc4ccccc4c23)c1]

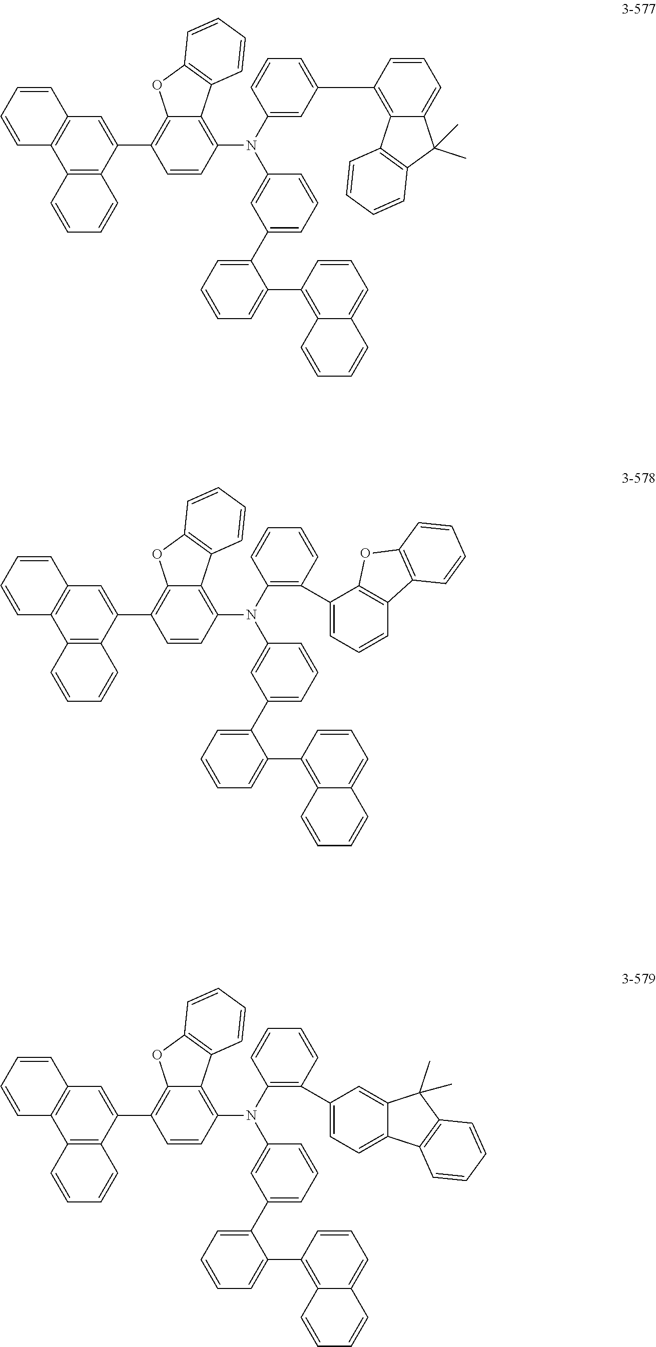 [SMILES: CC1(C)c2ccccc2-c2c(-c3cccc(N(c4cccc(-c5ccccc5-c5cccc6ccccc56)c4)c4ccc(-c5cc6ccccc6c6ccccc56)c5oc6ccccc6c45)c3)cccc21 ; c1cc(-c2ccccc2-c2cccc3ccccc23)cc(N(c2ccccc2-c2cccc3c2oc2ccccc23)c2ccc(-c3cc4ccccc4c4ccccc34)c3oc4ccccc4c23)c1 ; CC1(C)c2ccccc2-c2ccc(-c3ccccc3N(c3cccc(-c4ccccc4-c4cccc5ccccc45)c3)c3ccc(-c4cc5ccccc5c5ccccc45)c4oc5ccccc5c34)cc21]

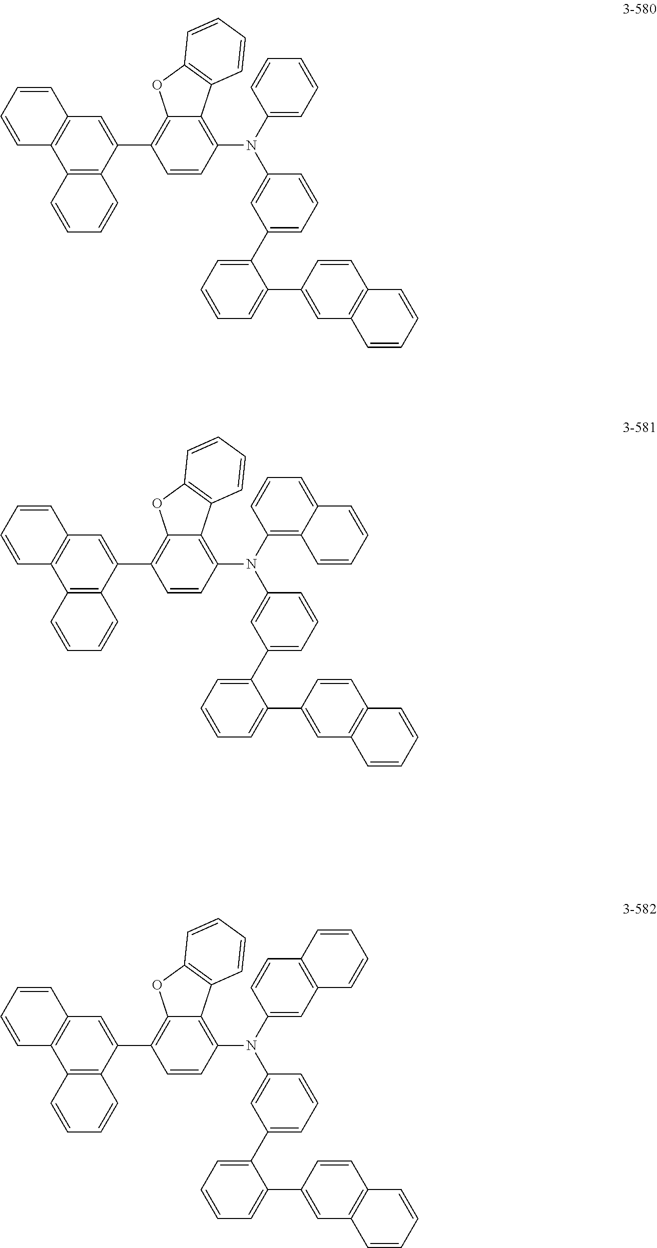 [SMILES: c1ccc(N(c2cccc(-c3ccccc3-c3ccc4ccccc4c3)c2)c2ccc(-c3cc4ccccc4c4ccccc34)c3oc4ccccc4c23)cc1 ; c1cc(-c2ccccc2-c2ccc3ccccc3c2)cc(N(c2cccc3ccccc23)c2ccc(-c3cc4ccccc4c4ccccc34)c3oc4ccccc4c23)c1 ; c1cc(-c2ccccc2-c2ccc3ccccc3c2)cc(N(c2ccc3ccccc3c2)c2ccc(-c3cc4ccccc4c4ccccc34)c3oc4ccccc4c23)c1]

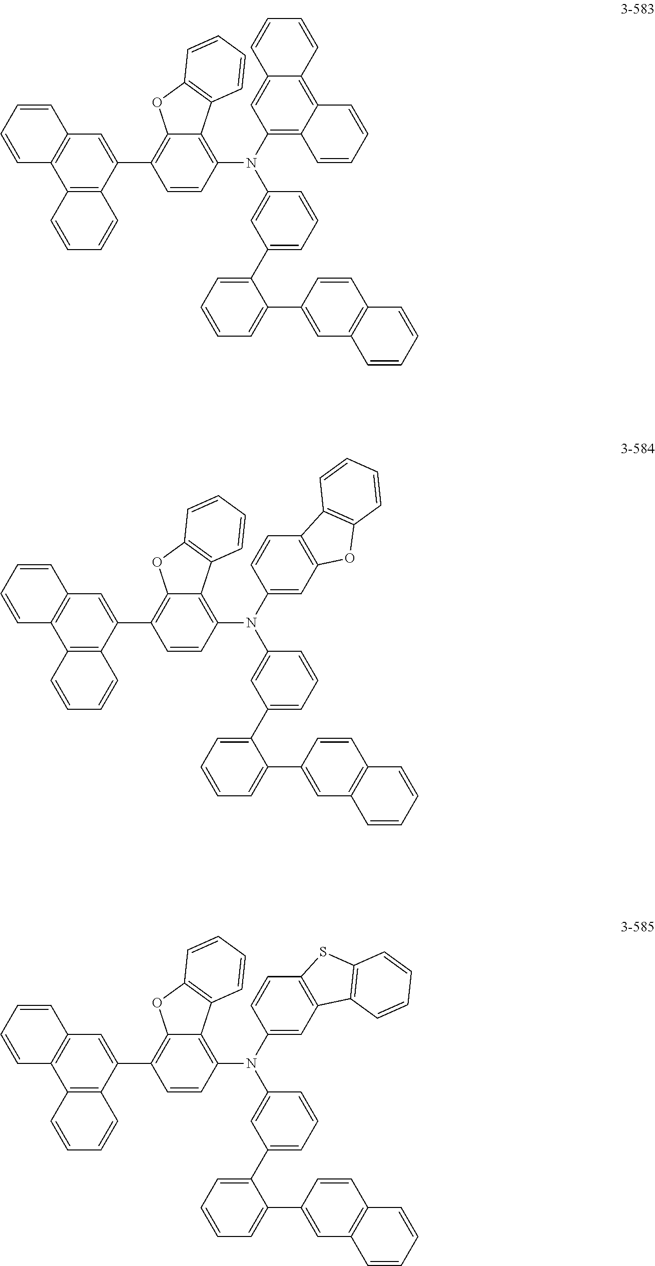 [SMILES: c1cc(-c2ccccc2-c2ccc3ccccc3c2)cc(N(c2cc3ccccc3c3ccccc23)c2ccc(-c3cc4ccccc4c4ccccc34)c3oc4ccccc4c23)c1 ; c1cc(-c2ccccc2-c2ccc3ccccc3c2)cc(N(c2ccc3c(c2)oc2ccccc23)c2ccc(-c3cc4ccccc4c4ccccc34)c3oc4ccccc4c23)c1 ; c1cc(-c2ccccc2-c2ccc3ccccc3c2)cc(N(c2ccc3sc4ccccc4c3c2)c2ccc(-c3cc4ccccc4c4ccccc34)c3oc4ccccc4c23)c1]

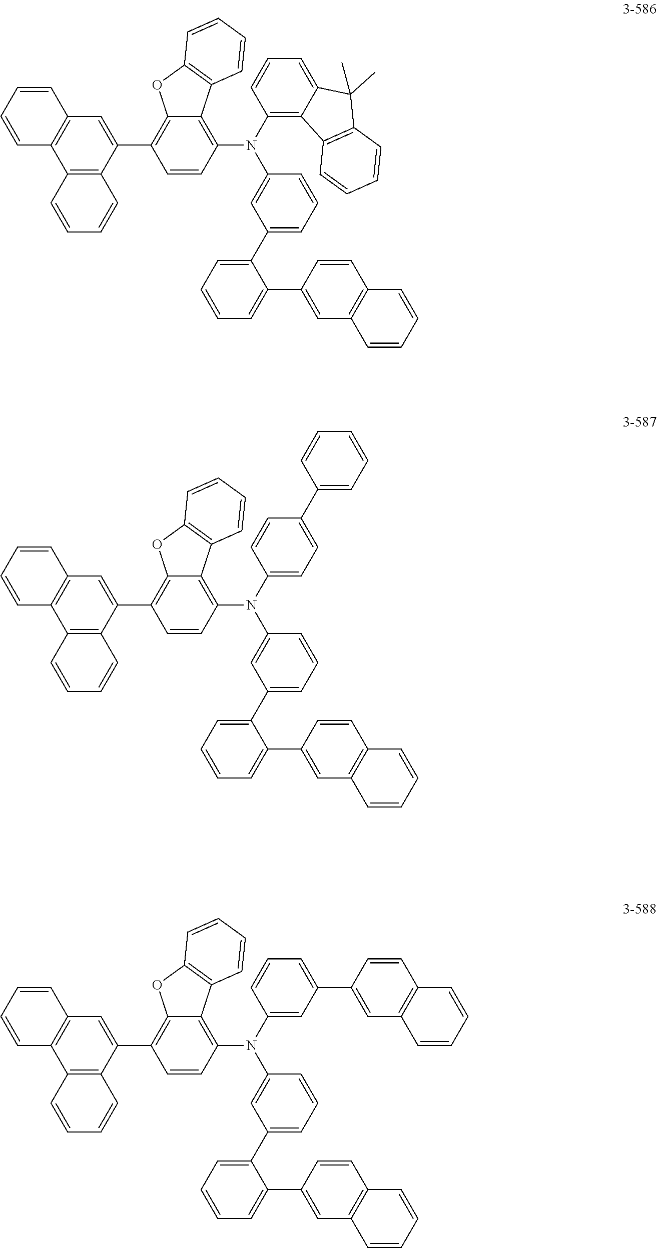 [SMILES: CC1(C)c2ccccc2-c2c(N(c3cccc(-c4ccccc4-c4ccc5ccccc5c4)c3)c3ccc(-c4cc5ccccc5c5ccccc45)c4oc5ccccc5c34)cccc21 ; c1ccc(-c2ccc(N(c3cccc(-c4ccccc4-c4ccc5ccccc5c4)c3)c3ccc(-c4cc5ccccc5c5ccccc45)c4oc5ccccc5c34)cc2)cc1 ; c1cc(-c2ccc3ccccc3c2)cc(N(c2cccc(-c3ccccc3-c3ccc4ccccc4c3)c2)c2ccc(-c3cc4ccccc4c4ccccc34)c3oc4ccccc4c23)c1]

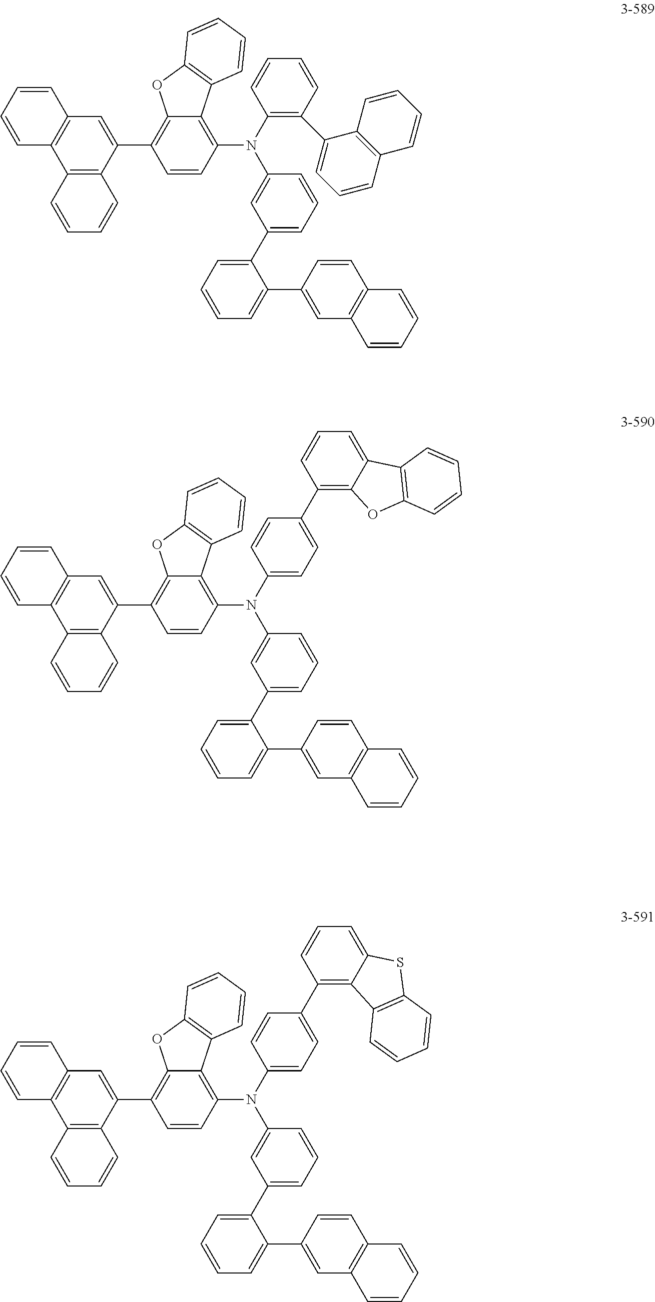 [SMILES: c1cc(-c2ccccc2-c2ccc3ccccc3c2)cc(N(c2ccccc2-c2cccc3ccccc23)c2ccc(-c3cc4ccccc4c4ccccc34)c3oc4ccccc4c23)c1 ; c1cc(-c2ccccc2-c2ccc3ccccc3c2)cc(N(c2ccc(-c3cccc4c3oc3ccccc34)cc2)c2ccc(-c3cc4ccccc4c4ccccc34)c3oc4ccccc4c23)c1 ; c1cc(-c2ccccc2-c2ccc3ccccc3c2)cc(N(c2ccc(-c3cccc4sc5ccccc5c34)cc2)c2ccc(-c3cc4ccccc4c4ccccc34)c3oc4ccccc4c23)c1]

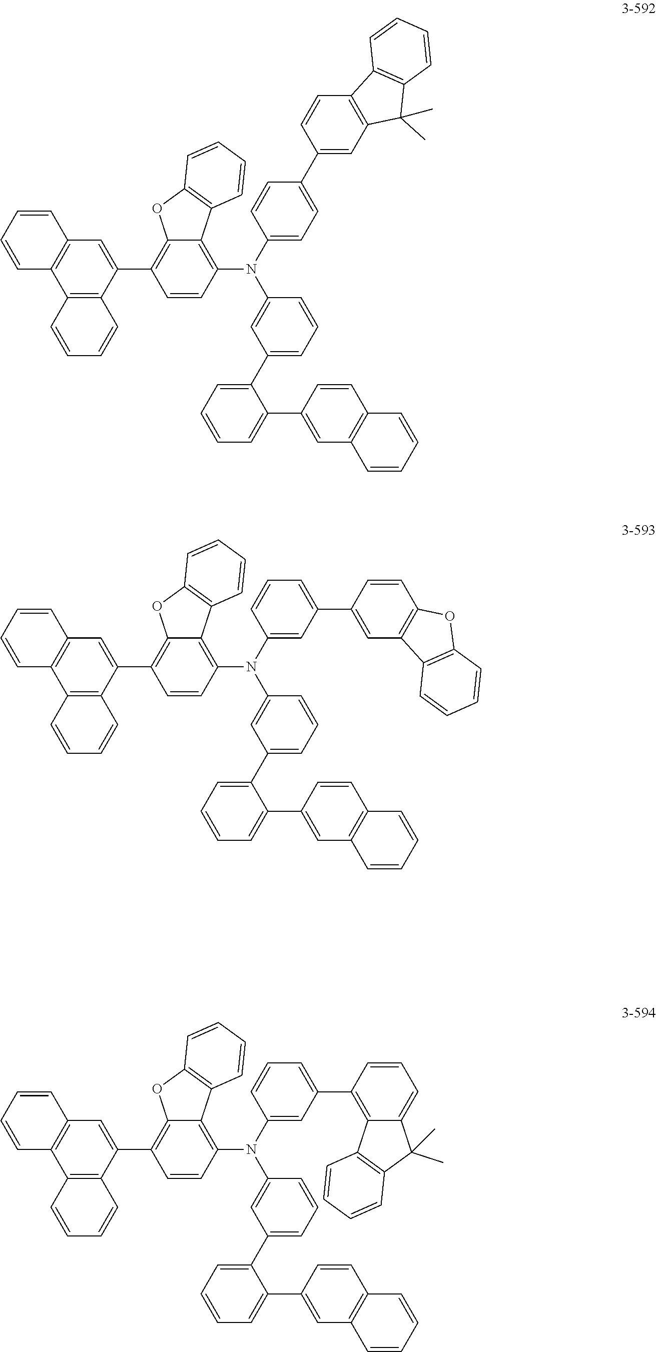 [SMILES: CC1(C)c2ccccc2-c2ccc(-c3ccc(N(c4cccc(-c5ccccc5-c5ccc6ccccc6c5)c4)c4ccc(-c5cc6ccccc6c6ccccc56)c5oc6ccccc6c45)cc3)cc21 ; c1cc(-c2ccc3oc4ccccc4c3c2)cc(N(c2cccc(-c3ccccc3-c3ccc4ccccc4c3)c2)c2ccc(-c3cc4ccccc4c4ccccc34)c3oc4ccccc4c23)c1 ; CC1(C)c2ccccc2-c2c(-c3cccc(N(c4cccc(-c5ccccc5-c5ccc6ccccc6c5)c4)c4ccc(-c5cc6ccccc6c6ccccc56)c5oc6ccccc6c45)c3)cccc21]

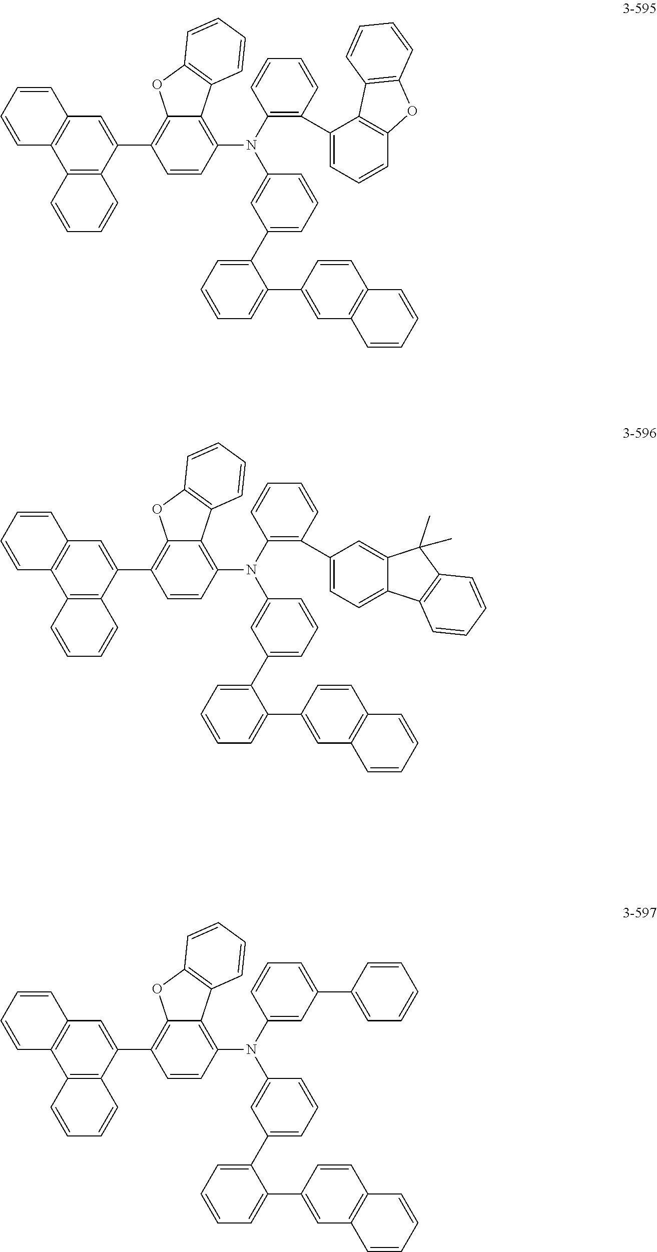 [SMILES: c1cc(-c2ccccc2-c2ccc3ccccc3c2)cc(N(c2ccccc2-c2cccc3oc4ccccc4c23)c2ccc(-c3cc4ccccc4c4ccccc34)c3oc4ccccc4c23)c1 ; CC1(C)c2ccccc2-c2ccc(-c3ccccc3N(c3cccc(-c4ccccc4-c4ccc5ccccc5c4)c3)c3ccc(-c4cc5ccccc5c5ccccc45)c4oc5ccccc5c34)cc21 ; c1ccc(-c2cccc(N(c3cccc(-c4ccccc4-c4ccc5ccccc5c4)c3)c3ccc(-c4cc5ccccc5c5ccccc45)c4oc5ccccc5c34)c2)cc1]

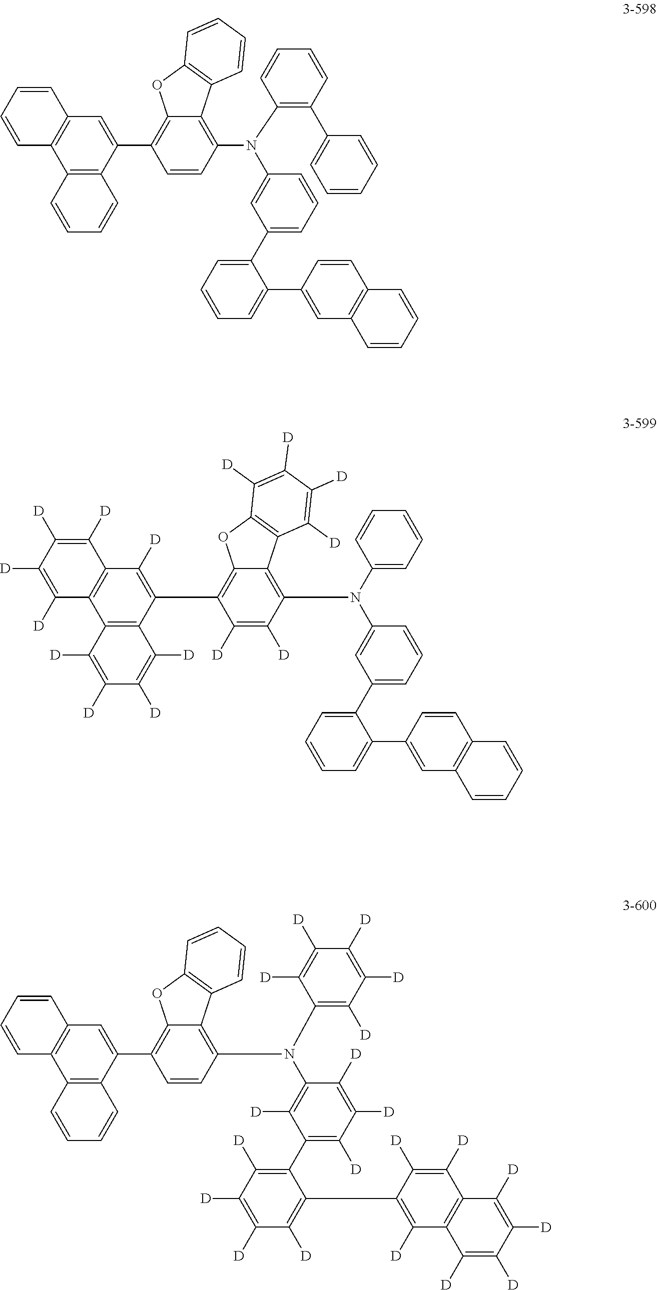 [SMILES: c1ccc(-c2ccccc2N(c2cccc(-c3ccccc3-c3ccc4ccccc4c3)c2)c2ccc(-c3cc4ccccc4c4ccccc34)c3oc4ccccc4c23)cc1 ; [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(N(c4ccccc4)c4cccc(-c5ccccc5-c5ccc6ccccc6c5)c4)c32)c1[2H] ; [2H]c1c([2H])c([2H])c(N(c2c([2H])c([2H])c([2H])c(-c3c([2H])c([2H])c([2H])c([2H])c3-c3c([2H])c([2H])c4c([2H])c([2H])c([2H])c([2H])c4c3[2H])c2[2H])c2ccc(-c3cc4ccccc4c4ccccc34)c3oc4ccccc4c23)c([2H])c1[2H]]

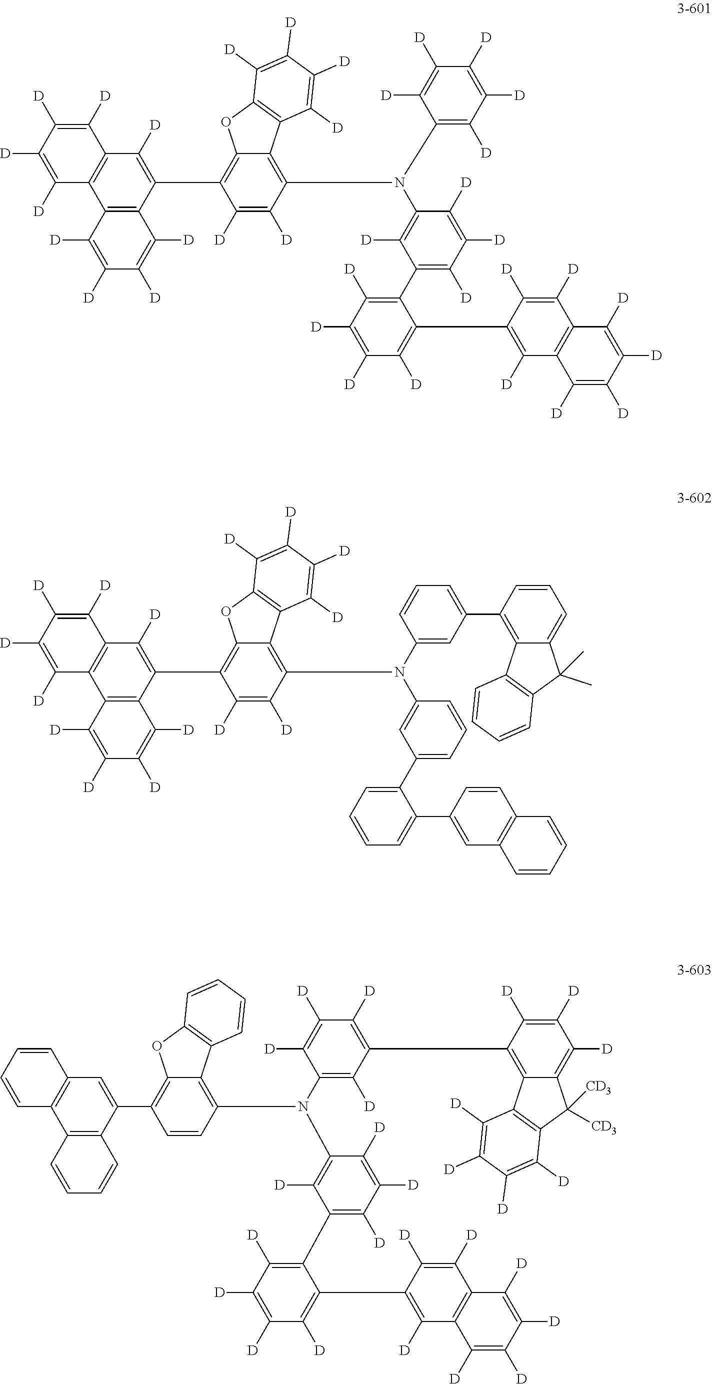

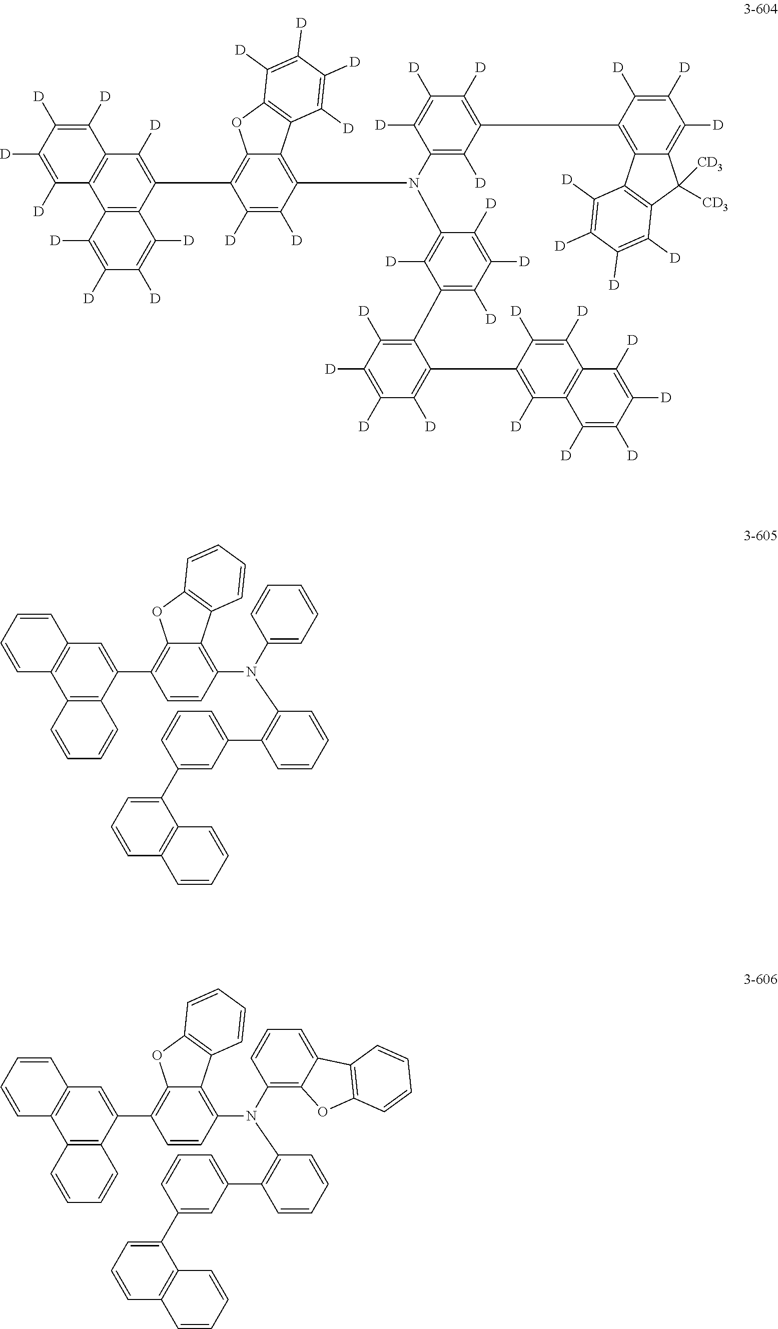

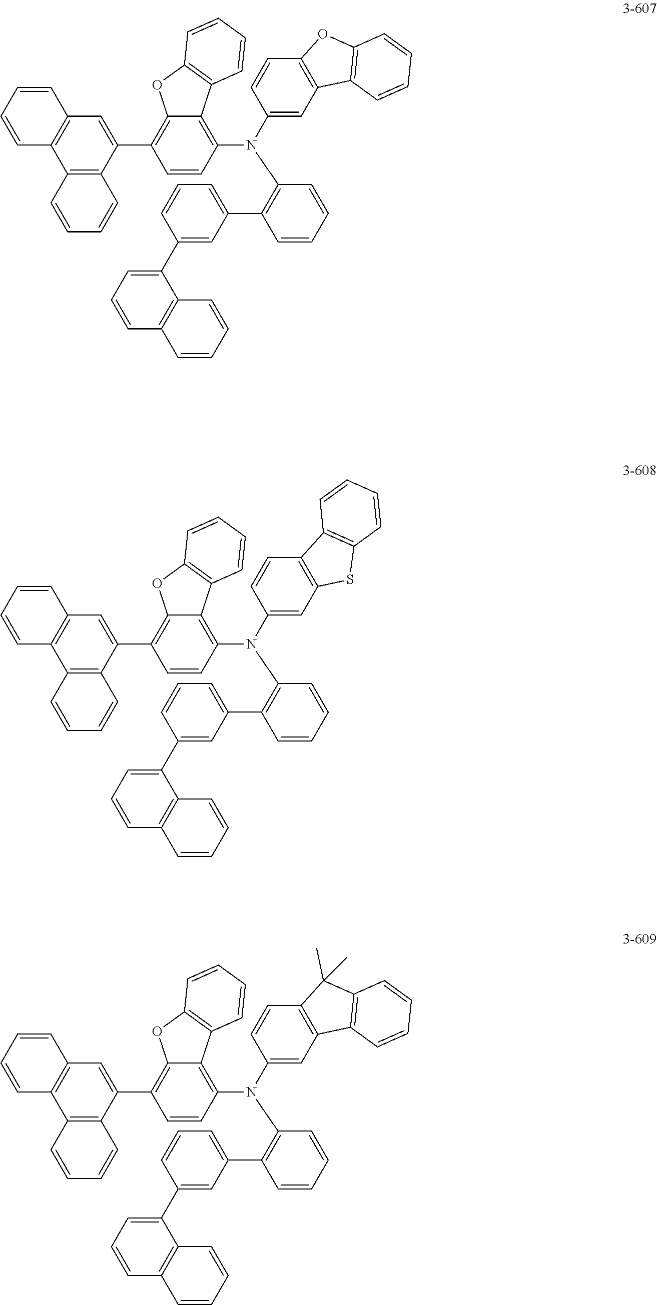 [SMILES: c1cc(-c2ccccc2N(c2ccc3oc4ccccc4c3c2)c2ccc(-c3cc4ccccc4c4ccccc34)c3oc4ccccc4c23)cc(-c2cccc3ccccc23)c1 ; c1cc(-c2ccccc2N(c2ccc3c(c2)sc2ccccc23)c2ccc(-c3cc4ccccc4c4ccccc34)c3oc4ccccc4c23)cc(-c2cccc3ccccc23)c1 ; CC1(C)c2ccccc2-c2cc(N(c3ccccc3-c3cccc(-c4cccc5ccccc45)c3)c3ccc(-c4cc5ccccc5c5ccccc45)c4oc5ccccc5c34)ccc21]

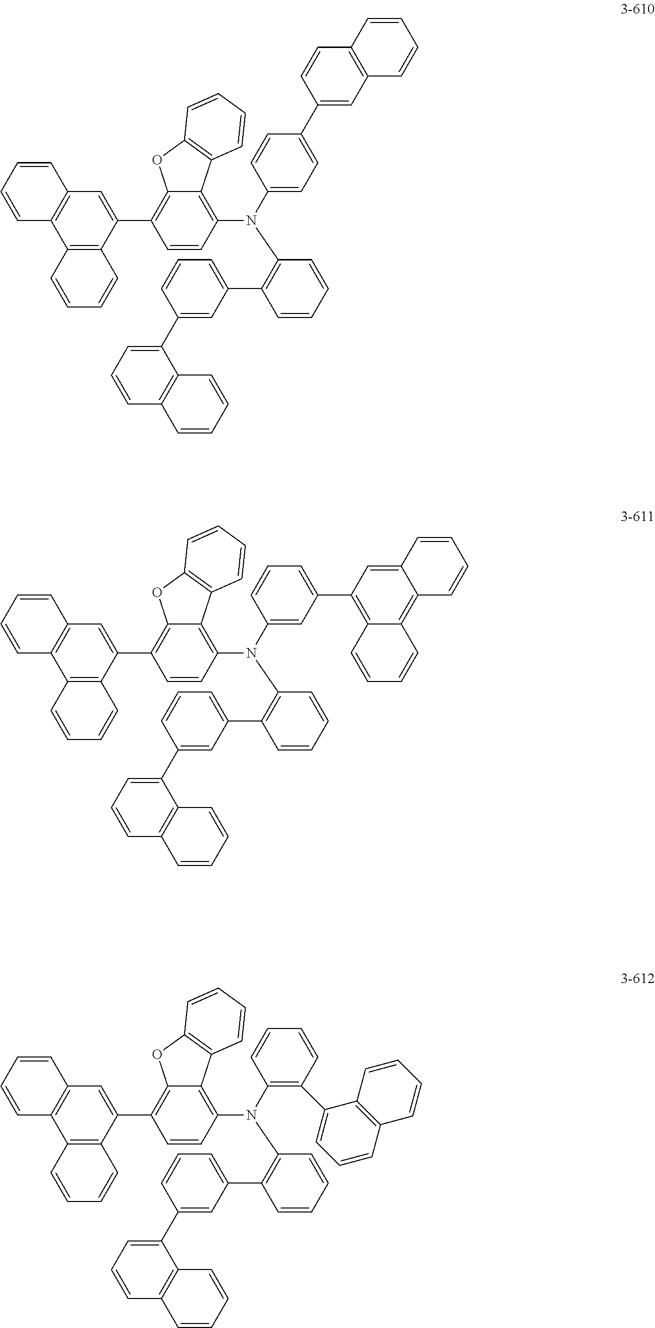 [SMILES: c1cc(-c2ccccc2N(c2ccc(-c3ccc4ccccc4c3)cc2)c2ccc(-c3cc4ccccc4c4ccccc34)c3oc4ccccc4c23)cc(-c2cccc3ccccc23)c1 ; c1cc(-c2ccccc2N(c2cccc(-c3cc4ccccc4c4ccccc34)c2)c2ccc(-c3cc4ccccc4c4ccccc34)c3oc4ccccc4c23)cc(-c2cccc3ccccc23)c1 ; c1cc(-c2ccccc2N(c2ccccc2-c2cccc3ccccc23)c2ccc(-c3cc4ccccc4c4ccccc34)c3oc4ccccc4c23)cc(-c2cccc3ccccc23)c1]

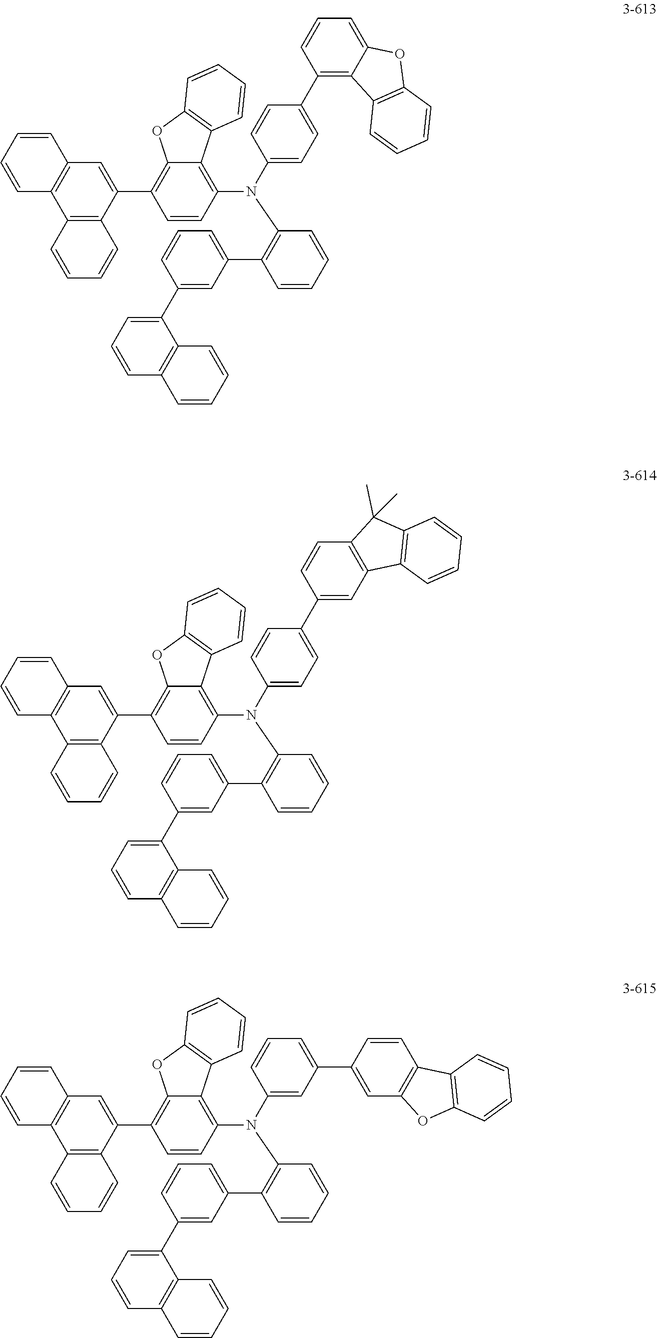 [SMILES: c1cc(-c2ccccc2N(c2ccc(-c3cccc4oc5ccccc5c34)cc2)c2ccc(-c3cc4ccccc4c4ccccc34)c3oc4ccccc4c23)cc(-c2cccc3ccccc23)c1 ; CC1(C)c2ccccc2-c2cc(-c3ccc(N(c4ccccc4-c4cccc(-c5cccc6ccccc56)c4)c4ccc(-c5cc6ccccc6c6ccccc56)c5oc6ccccc6c45)cc3)ccc21 ; c1cc(-c2ccccc2N(c2cccc(-c3ccc4c(c3)oc3ccccc34)c2)c2ccc(-c3cc4ccccc4c4ccccc34)c3oc4ccccc4c23)cc(-c2cccc3ccccc23)c1]

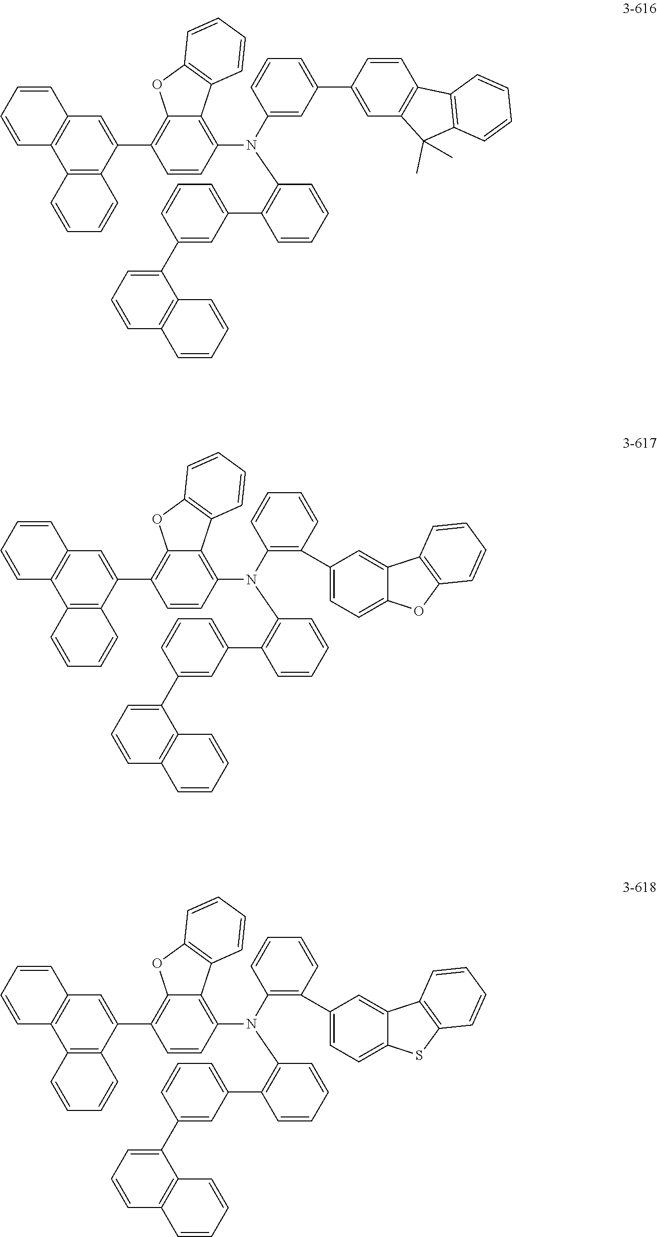 [SMILES: CC1(C)c2ccccc2-c2ccc(-c3cccc(N(c4ccccc4-c4cccc(-c5cccc6ccccc56)c4)c4ccc(-c5cc6ccccc6c6ccccc56)c5oc6ccccc6c45)c3)cc21 ; c1cc(-c2ccccc2N(c2ccccc2-c2ccc3oc4ccccc4c3c2)c2ccc(-c3cc4ccccc4c4ccccc34)c3oc4ccccc4c23)cc(-c2cccc3ccccc23)c1 ; c1cc(-c2ccccc2N(c2ccccc2-c2ccc3sc4ccccc4c3c2)c2ccc(-c3cc4ccccc4c4ccccc34)c3oc4ccccc4c23)cc(-c2cccc3ccccc23)c1]

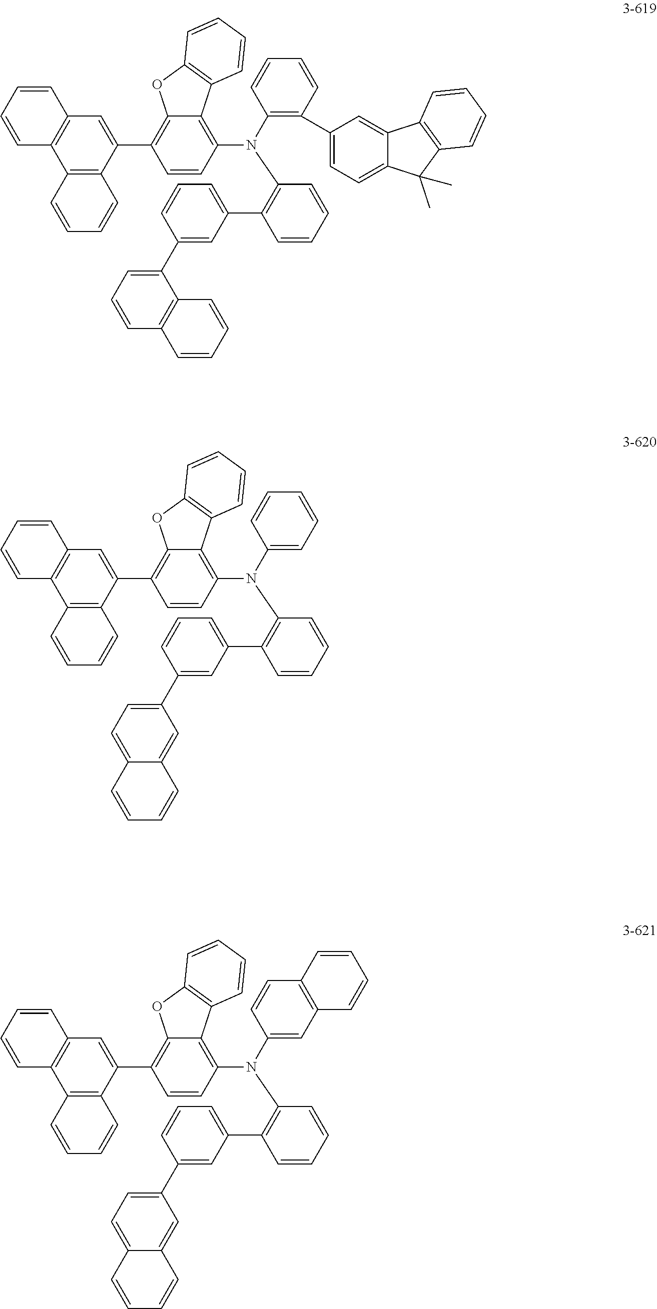 [SMILES: CC1(C)c2ccccc2-c2cc(-c3ccccc3N(c3ccccc3-c3cccc(-c4cccc5ccccc45)c3)c3ccc(-c4cc5ccccc5c5ccccc45)c4oc5ccccc5c34)ccc21 ; c1ccc(N(c2ccccc2-c2cccc(-c3ccc4ccccc4c3)c2)c2ccc(-c3cc4ccccc4c4ccccc34)c3oc4ccccc4c23)cc1 ; c1cc(-c2ccc3ccccc3c2)cc(-c2ccccc2N(c2ccc3ccccc3c2)c2ccc(-c3cc4ccccc4c4ccccc34)c3oc4ccccc4c23)c1]

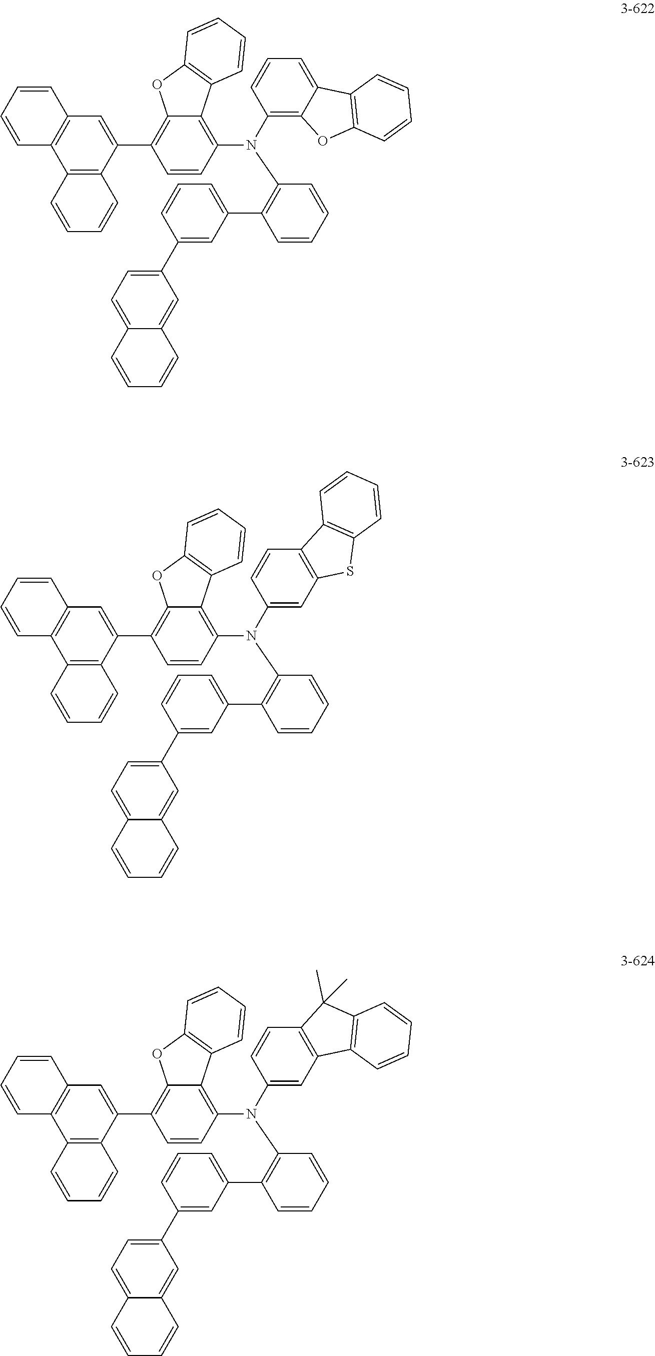 [SMILES: c1cc(-c2ccc3ccccc3c2)cc(-c2ccccc2N(c2cccc3c2oc2ccccc23)c2ccc(-c3cc4ccccc4c4ccccc34)c3oc4ccccc4c23)c1 ; c1cc(-c2ccc3ccccc3c2)cc(-c2ccccc2N(c2ccc3c(c2)sc2ccccc23)c2ccc(-c3cc4ccccc4c4ccccc34)c3oc4ccccc4c23)c1 ; CC1(C)c2ccccc2-c2cc(N(c3ccccc3-c3cccc(-c4ccc5ccccc5c4)c3)c3ccc(-c4cc5ccccc5c5ccccc45)c4oc5ccccc5c34)ccc21]

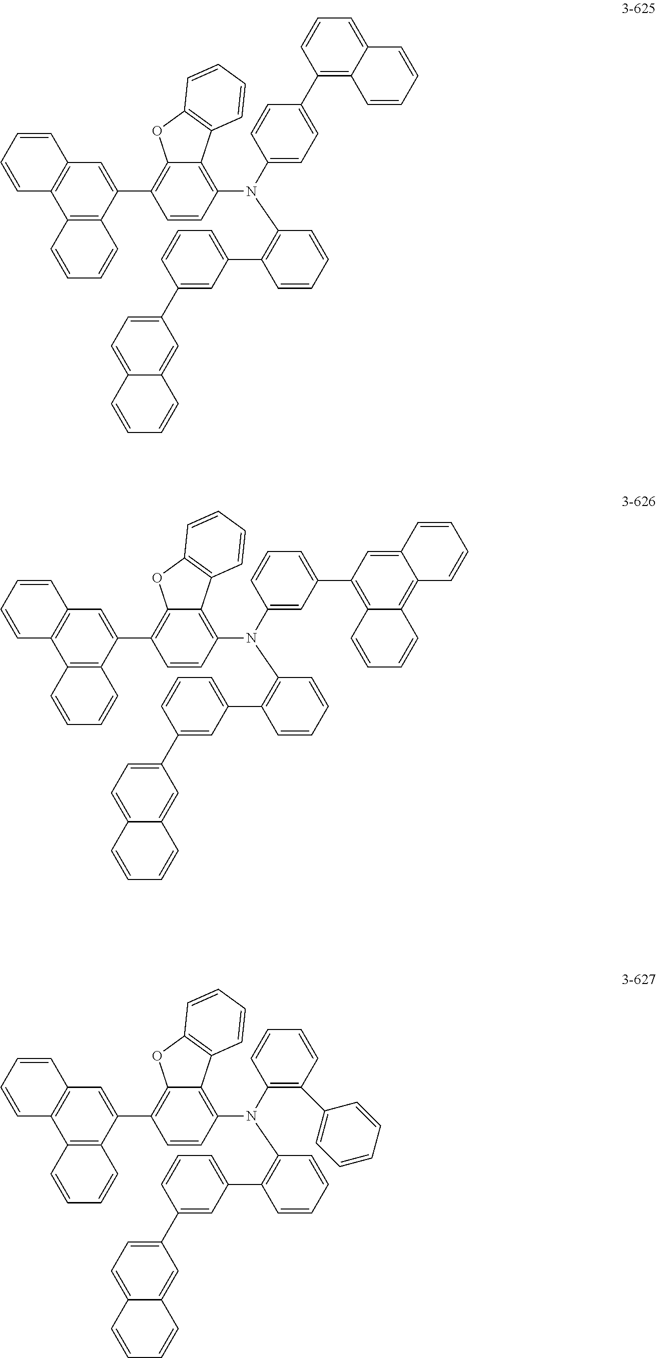 [SMILES: c1cc(-c2ccc3ccccc3c2)cc(-c2ccccc2N(c2ccc(-c3cccc4ccccc34)cc2)c2ccc(-c3cc4ccccc4c4ccccc34)c3oc4ccccc4c23)c1 ; c1cc(-c2ccc3ccccc3c2)cc(-c2ccccc2N(c2cccc(-c3cc4ccccc4c4ccccc34)c2)c2ccc(-c3cc4ccccc4c4ccccc34)c3oc4ccccc4c23)c1 ; c1ccc(-c2ccccc2N(c2ccccc2-c2cccc(-c3ccc4ccccc4c3)c2)c2ccc(-c3cc4ccccc4c4ccccc34)c3oc4ccccc4c23)cc1]

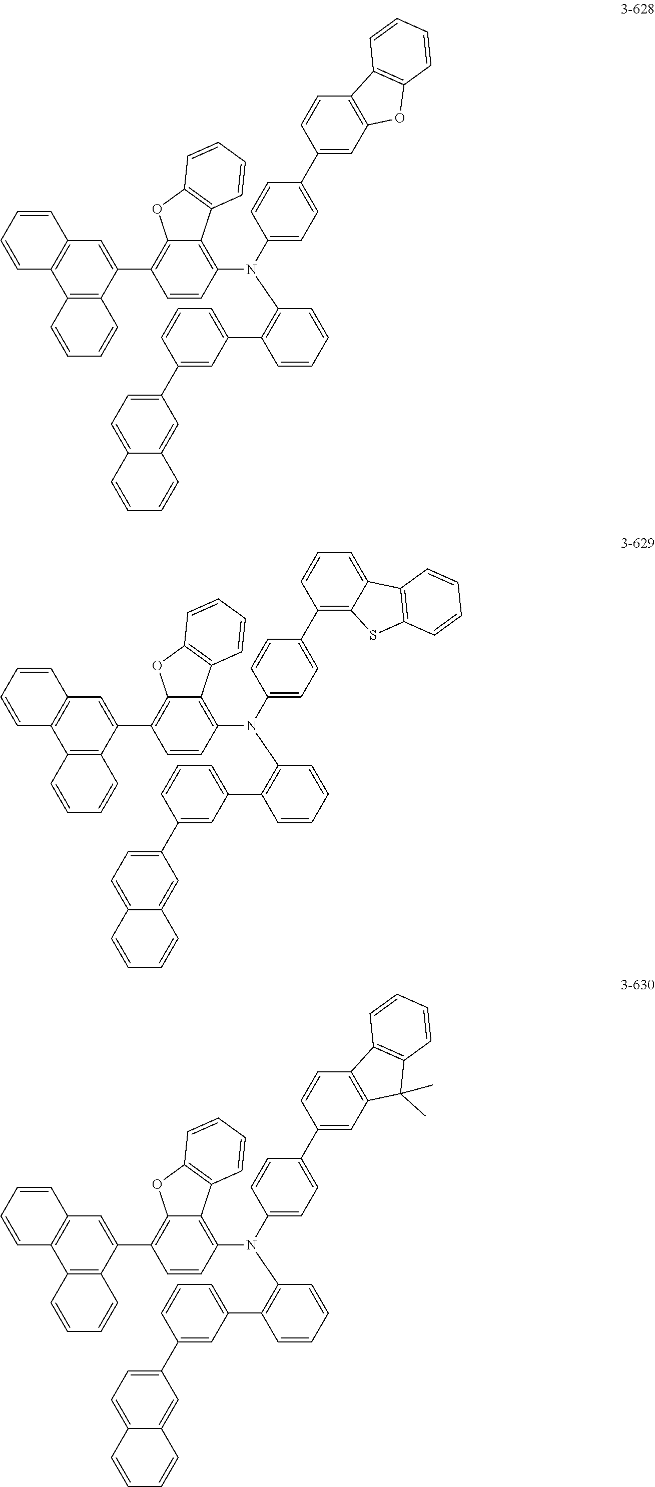 [SMILES: c1cc(-c2ccc3ccccc3c2)cc(-c2ccccc2N(c2ccc(-c3ccc4c(c3)oc3ccccc34)cc2)c2ccc(-c3cc4ccccc4c4ccccc34)c3oc4ccccc4c23)c1 ; c1cc(-c2ccc3ccccc3c2)cc(-c2ccccc2N(c2ccc(-c3cccc4c3sc3ccccc34)cc2)c2ccc(-c3cc4ccccc4c4ccccc34)c3oc4ccccc4c23)c1 ; CC1(C)c2ccccc2-c2ccc(-c3ccc(N(c4ccccc4-c4cccc(-c5ccc6ccccc6c5)c4)c4ccc(-c5cc6ccccc6c6ccccc56)c5oc6ccccc6c45)cc3)cc21]

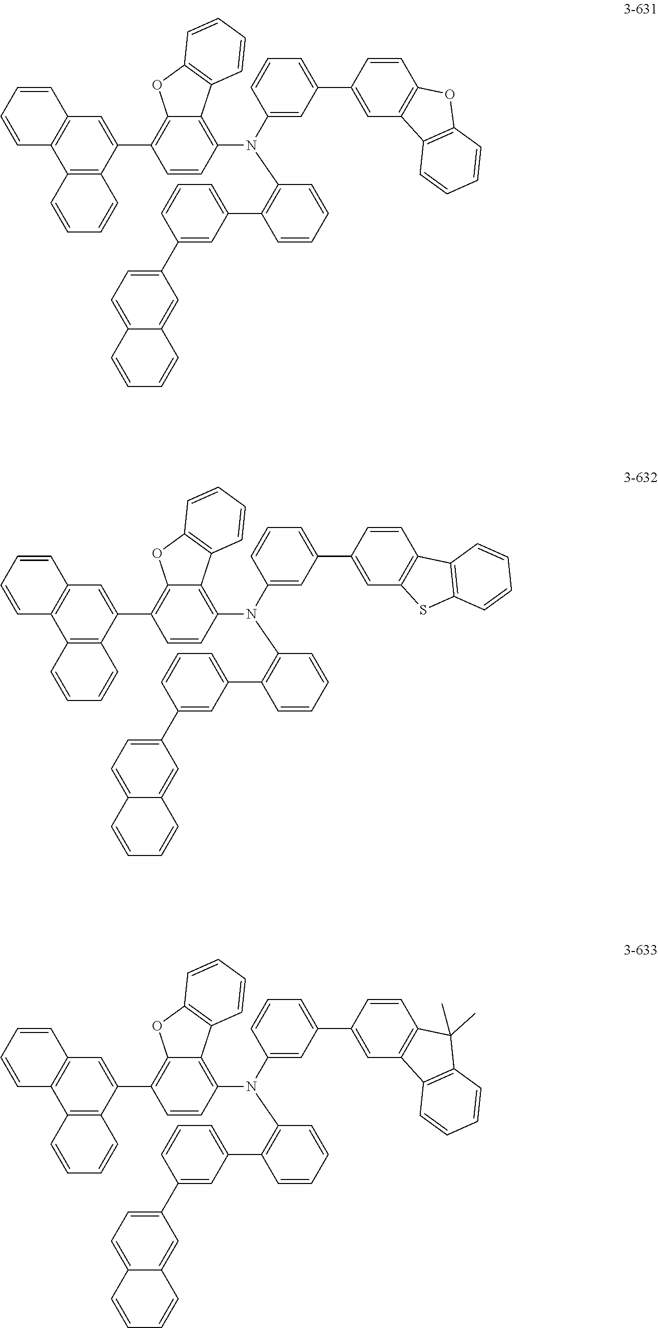 [SMILES: c1cc(-c2ccc3ccccc3c2)cc(-c2ccccc2N(c2cccc(-c3ccc4oc5ccccc5c4c3)c2)c2ccc(-c3cc4ccccc4c4ccccc34)c3oc4ccccc4c23)c1 ; c1cc(-c2ccc3ccccc3c2)cc(-c2ccccc2N(c2cccc(-c3ccc4c(c3)sc3ccccc34)c2)c2ccc(-c3cc4ccccc4c4ccccc34)c3oc4ccccc4c23)c1 ; CC1(C)c2ccccc2-c2cc(-c3cccc(N(c4ccccc4-c4cccc(-c5ccc6ccccc6c5)c4)c4ccc(-c5cc6ccccc6c6ccccc56)c5oc6ccccc6c45)c3)ccc21]

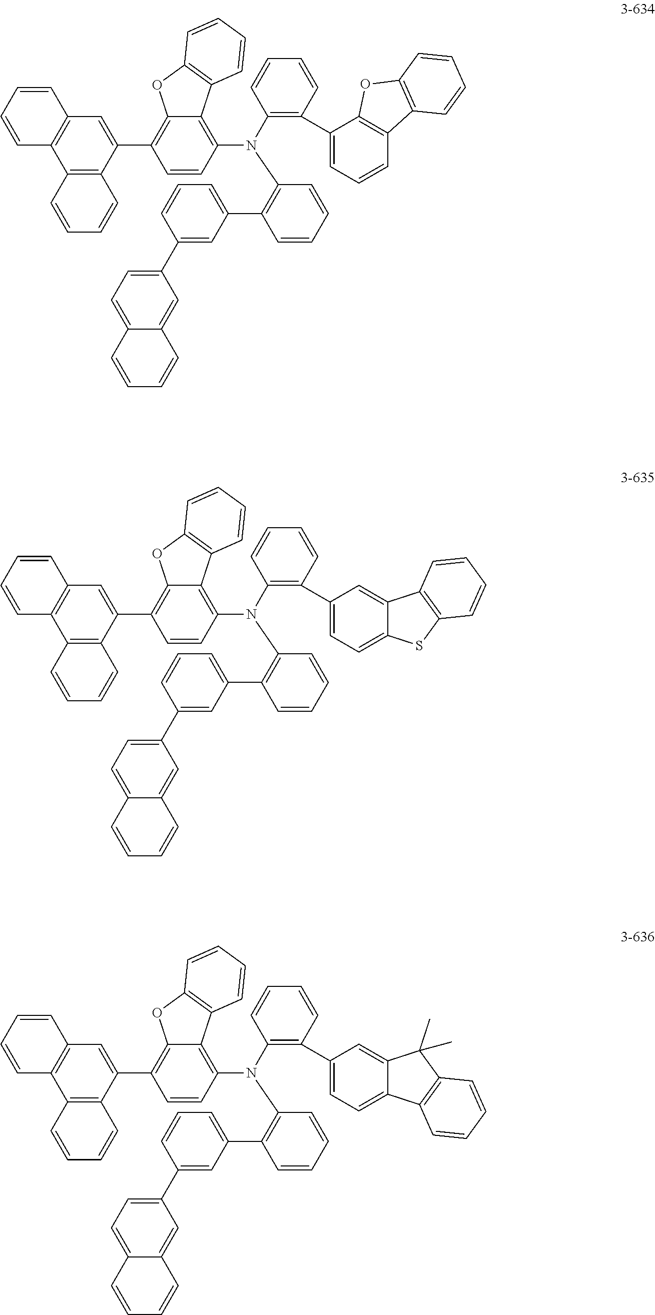 [SMILES: c1cc(-c2ccc3ccccc3c2)cc(-c2ccccc2N(c2ccccc2-c2cccc3c2oc2ccccc23)c2ccc(-c3cc4ccccc4c4ccccc34)c3oc4ccccc4c23)c1 ; c1cc(-c2ccc3ccccc3c2)cc(-c2ccccc2N(c2ccccc2-c2ccc3sc4ccccc4c3c2)c2ccc(-c3cc4ccccc4c4ccccc34)c3oc4ccccc4c23)c1 ; CC1(C)c2ccccc2-c2ccc(-c3ccccc3N(c3ccccc3-c3cccc(-c4ccc5ccccc5c4)c3)c3ccc(-c4cc5ccccc5c5ccccc45)c4oc5ccccc5c34)cc21]

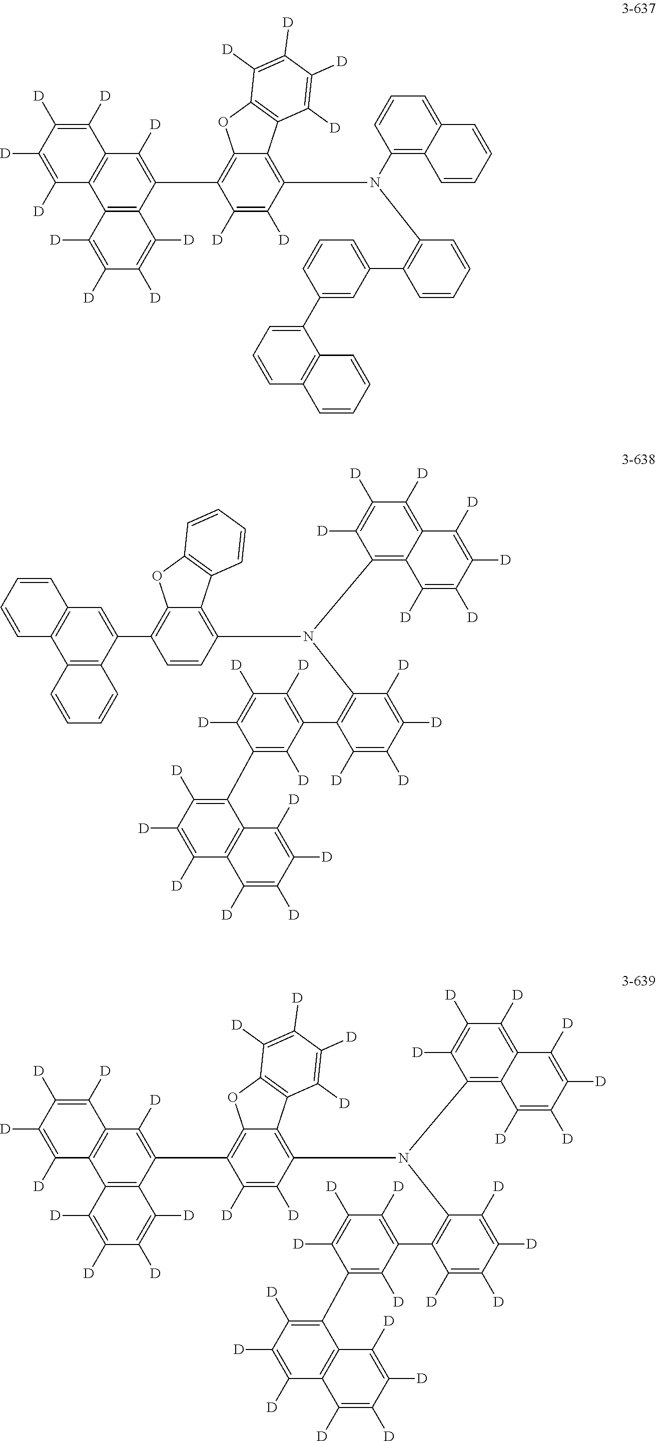

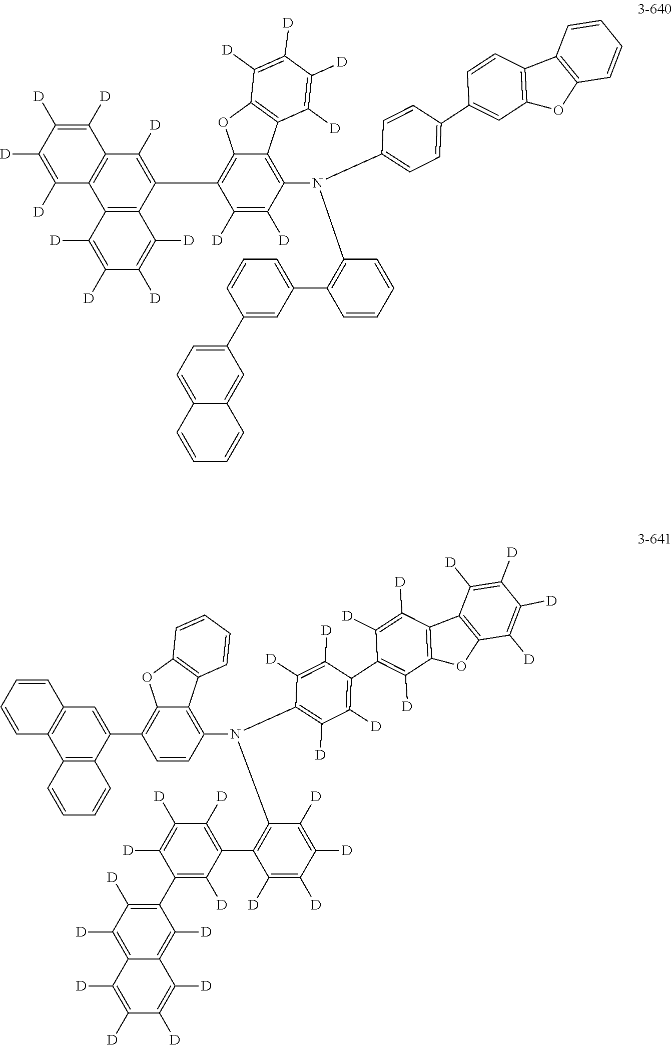 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(N(c4ccc(-c5ccc6c(c5)oc5ccccc56)cc4)c4ccccc4-c4cccc(-c5ccc6ccccc6c5)c4)c32)c1[2H] ; [2H]c1c([2H])c(-c2c([2H])c([2H])c([2H])c([2H])c2N(c2c([2H])c([2H])c(-c3c([2H])c([2H])c4c(oc5c([2H])c([2H])c([2H])c([2H])c54)c3[2H])c([2H])c2[2H])c2ccc(-c3cc4ccccc4c4ccccc34)c3oc4ccccc4c23)c([2H])c(-c2c([2H])c([2H])c3c([2H])c([2H])c([2H])c([2H])c3c2[2H])c1[2H]]

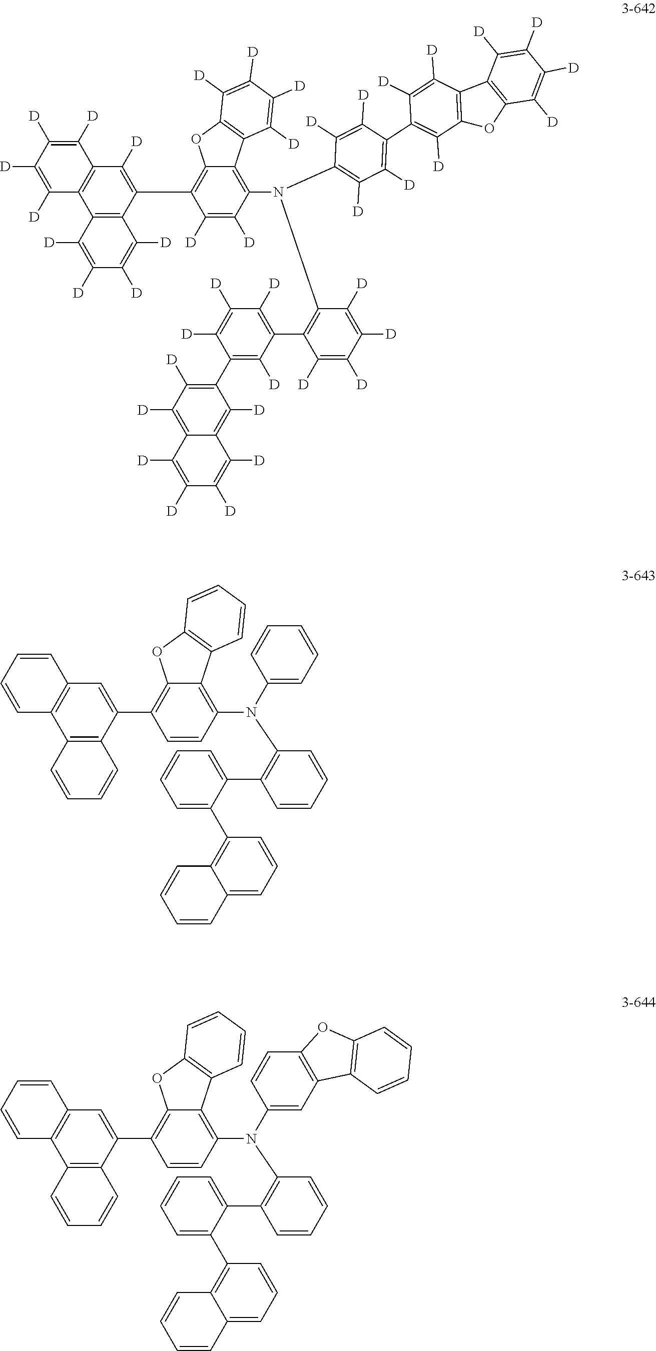

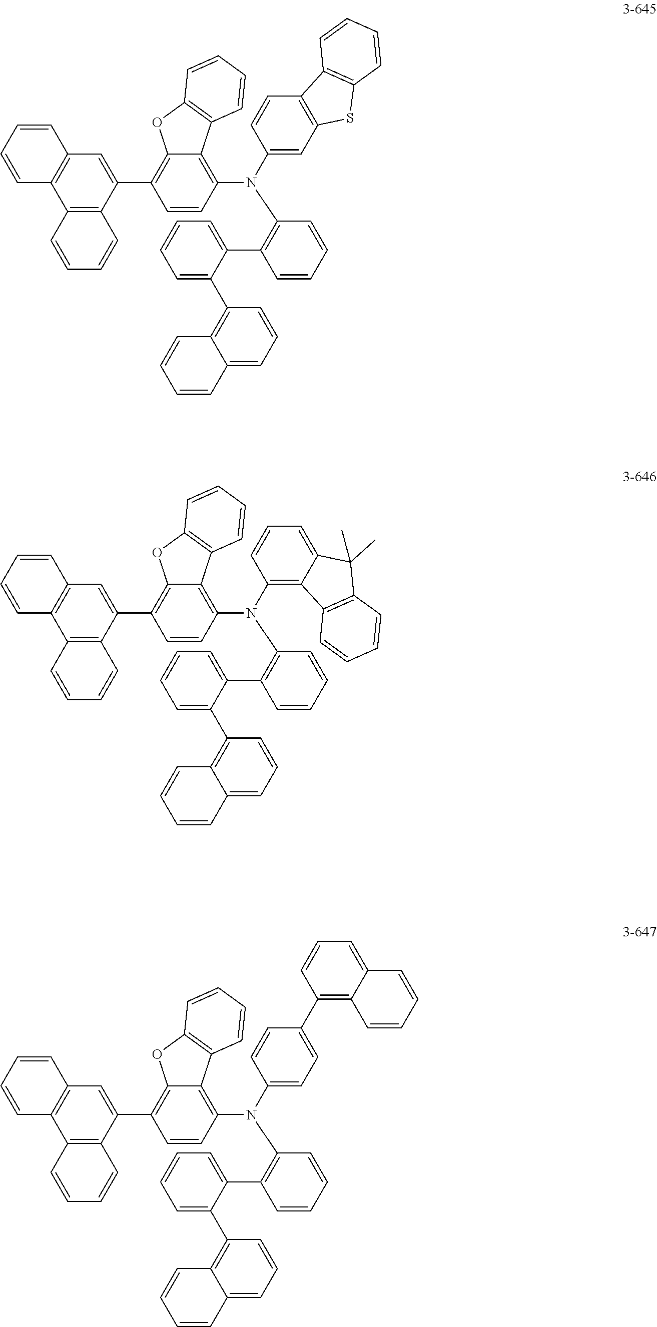 [SMILES: c1ccc(-c2cccc3ccccc23)c(-c2ccccc2N(c2ccc3c(c2)sc2ccccc23)c2ccc(-c3cc4ccccc4c4ccccc34)c3oc4ccccc4c23)c1 ; CC1(C)c2ccccc2-c2c(N(c3ccccc3-c3ccccc3-c3cccc4ccccc34)c3ccc(-c4cc5ccccc5c5ccccc45)c4oc5ccccc5c34)cccc21 ; c1ccc(-c2cccc3ccccc23)c(-c2ccccc2N(c2ccc(-c3cccc4ccccc34)cc2)c2ccc(-c3cc4ccccc4c4ccccc34)c3oc4ccccc4c23)c1]

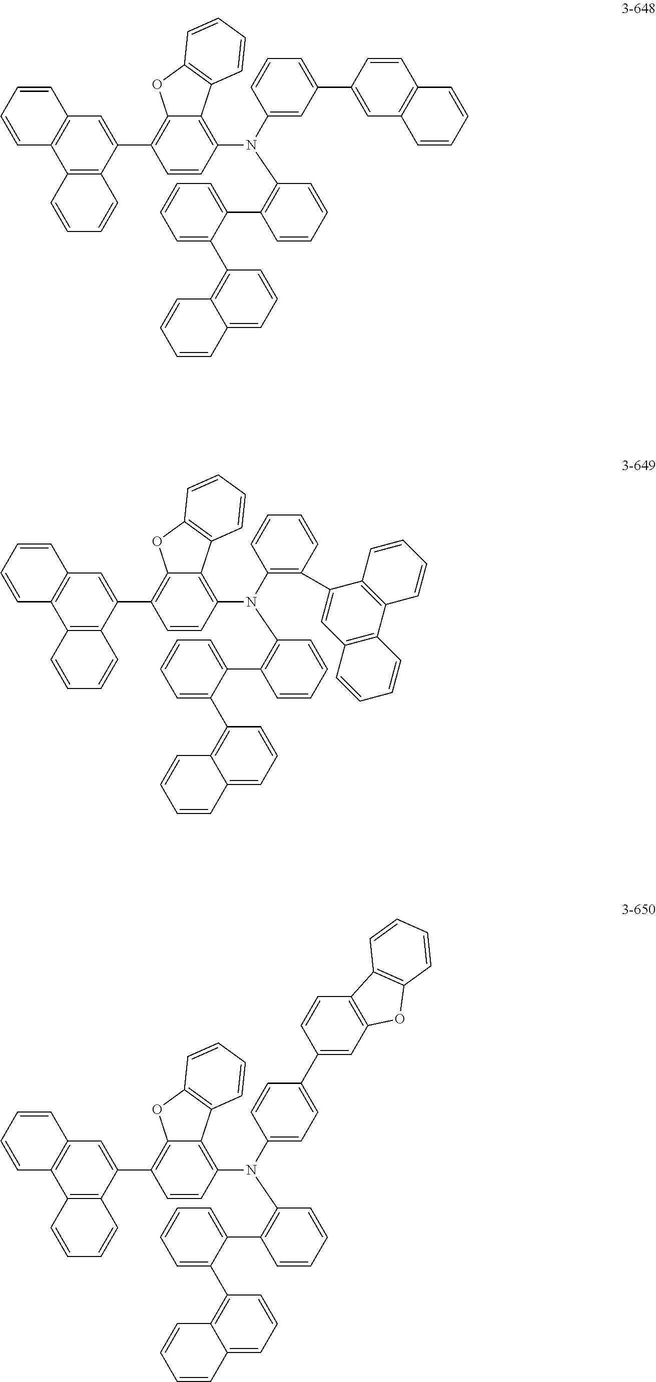 [SMILES: c1cc(-c2ccc3ccccc3c2)cc(N(c2ccccc2-c2ccccc2-c2cccc3ccccc23)c2ccc(-c3cc4ccccc4c4ccccc34)c3oc4ccccc4c23)c1 ; c1ccc(-c2cccc3ccccc23)c(-c2ccccc2N(c2ccccc2-c2cc3ccccc3c3ccccc23)c2ccc(-c3cc4ccccc4c4ccccc34)c3oc4ccccc4c23)c1 ; c1ccc(-c2cccc3ccccc23)c(-c2ccccc2N(c2ccc(-c3ccc4c(c3)oc3ccccc34)cc2)c2ccc(-c3cc4ccccc4c4ccccc34)c3oc4ccccc4c23)c1]

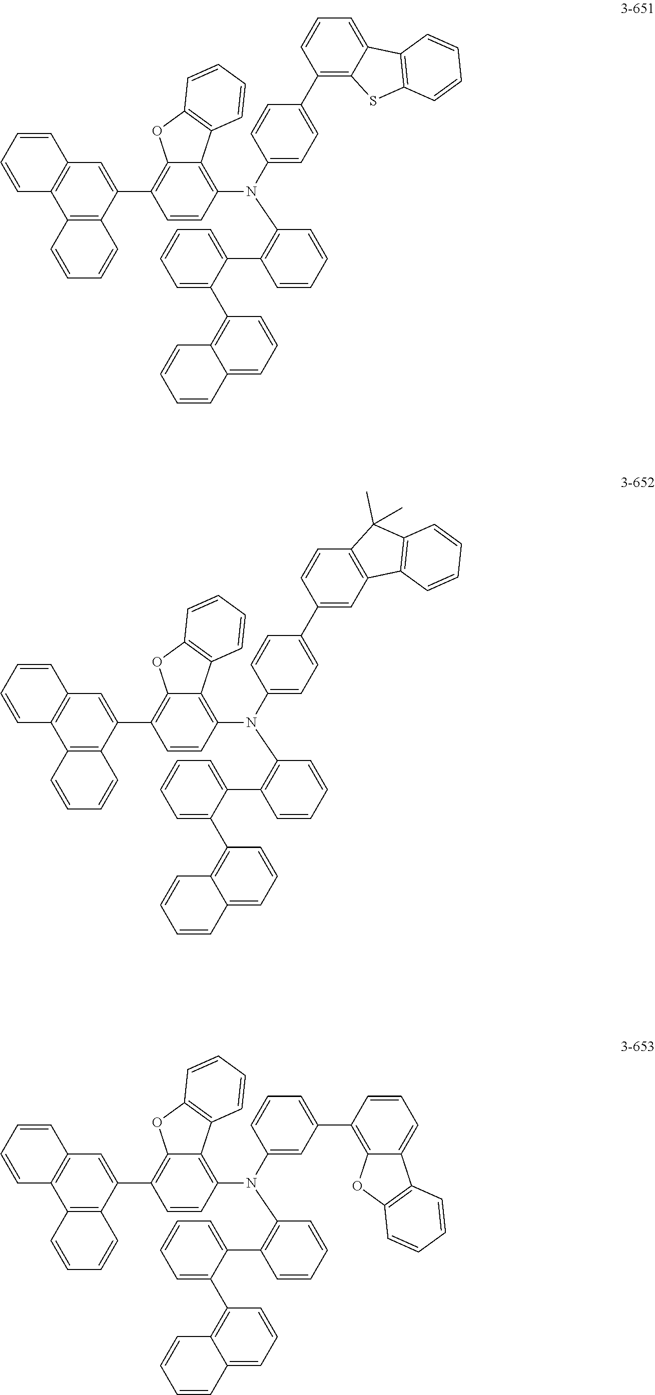 [SMILES: c1ccc(-c2cccc3ccccc23)c(-c2ccccc2N(c2ccc(-c3cccc4c3sc3ccccc34)cc2)c2ccc(-c3cc4ccccc4c4ccccc34)c3oc4ccccc4c23)c1 ; CC1(C)c2ccccc2-c2cc(-c3ccc(N(c4ccccc4-c4ccccc4-c4cccc5ccccc45)c4ccc(-c5cc6ccccc6c6ccccc56)c5oc6ccccc6c45)cc3)ccc21 ; c1cc(-c2cccc3c2oc2ccccc23)cc(N(c2ccccc2-c2ccccc2-c2cccc3ccccc23)c2ccc(-c3cc4ccccc4c4ccccc34)c3oc4ccccc4c23)c1]

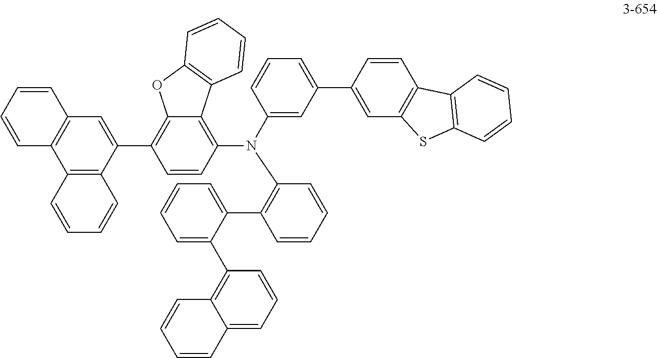 [SMILES: c1cc(-c2ccc3c(c2)sc2ccccc23)cc(N(c2ccccc2-c2ccccc2-c2cccc3ccccc23)c2ccc(-c3cc4ccccc4c4ccccc34)c3oc4ccccc4c23)c1]

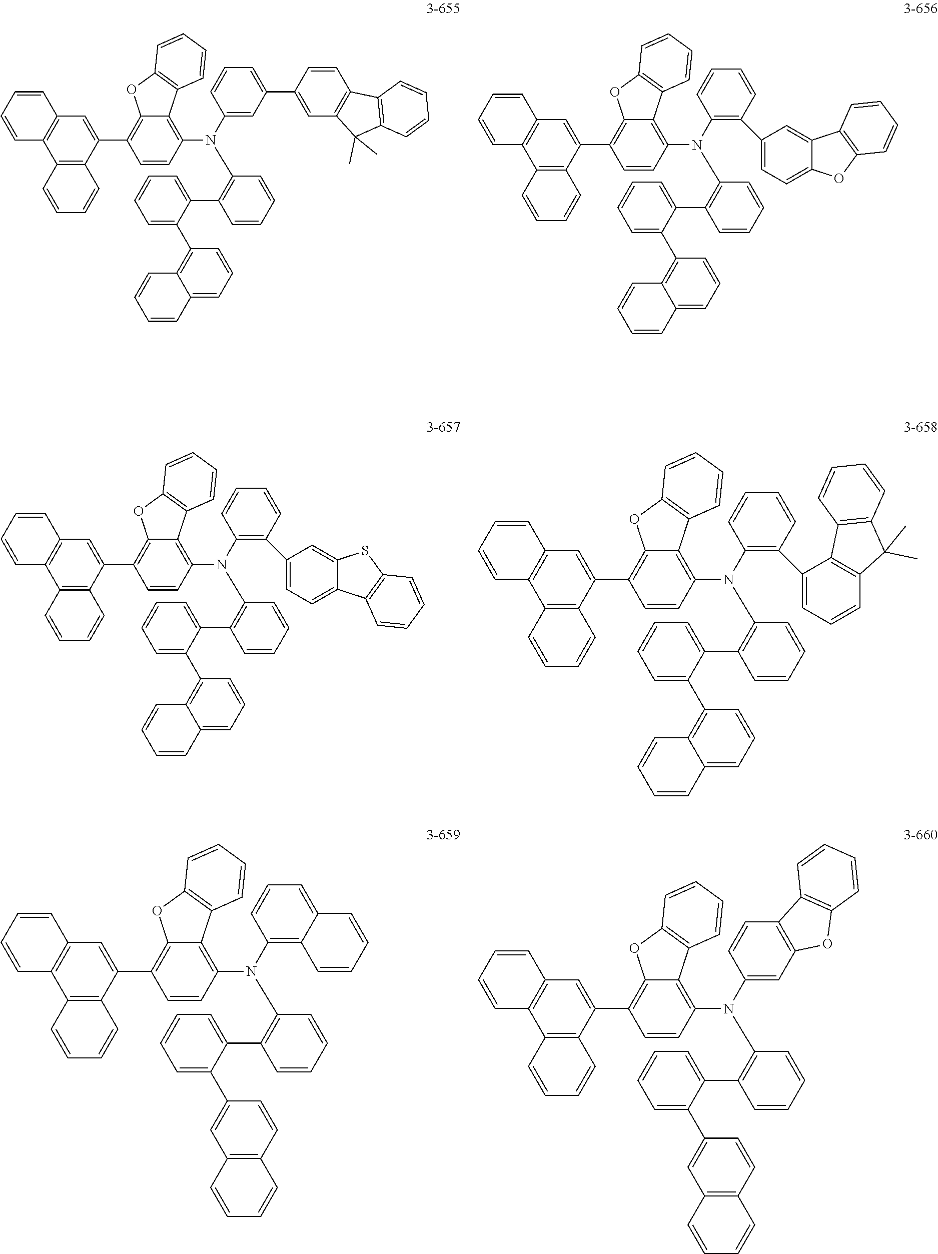

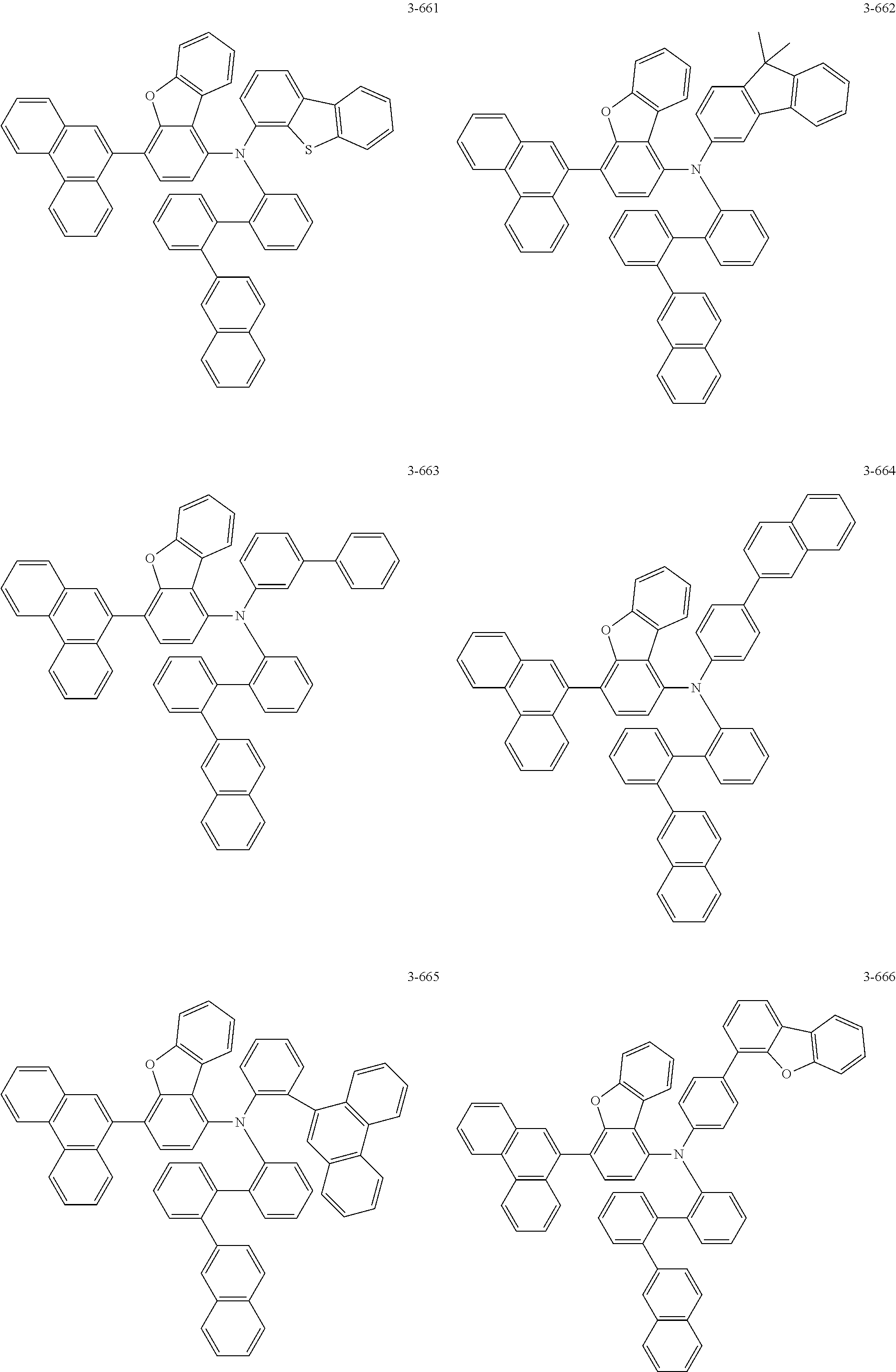

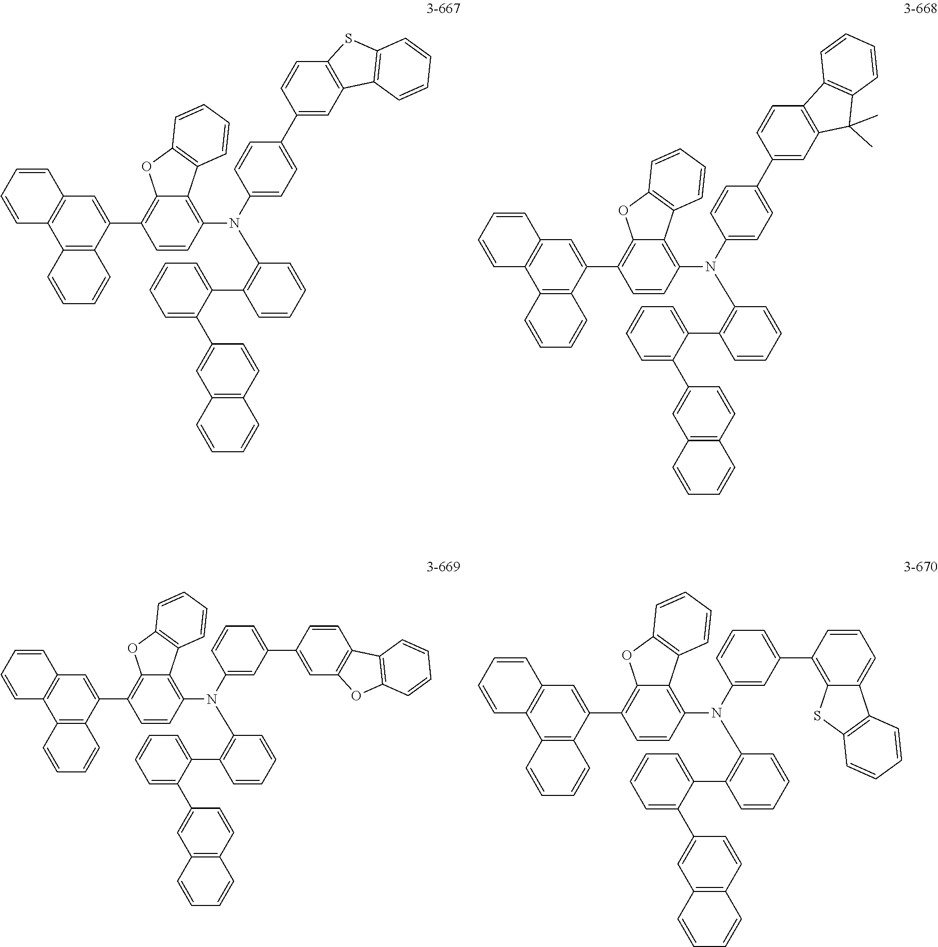 [SMILES: c1ccc(-c2ccccc2N(c2ccc(-c3ccc4sc5ccccc5c4c3)cc2)c2ccc(-c3cc4ccccc4c4ccccc34)c3oc4ccccc4c23)c(-c2ccc3ccccc3c2)c1 ; CC1(C)c2ccccc2-c2ccc(-c3ccc(N(c4ccccc4-c4ccccc4-c4ccc5ccccc5c4)c4ccc(-c5cc6ccccc6c6ccccc56)c5oc6ccccc6c45)cc3)cc21 ; c1cc(-c2ccc3c(c2)oc2ccccc23)cc(N(c2ccccc2-c2ccccc2-c2ccc3ccccc3c2)c2ccc(-c3cc4ccccc4c4ccccc34)c3oc4ccccc4c23)c1 ; c1cc(-c2cccc3c2sc2ccccc23)cc(N(c2ccccc2-c2ccccc2-c2ccc3ccccc3c2)c2ccc(-c3cc4ccccc4c4ccccc34)c3oc4ccccc4c23)c1]

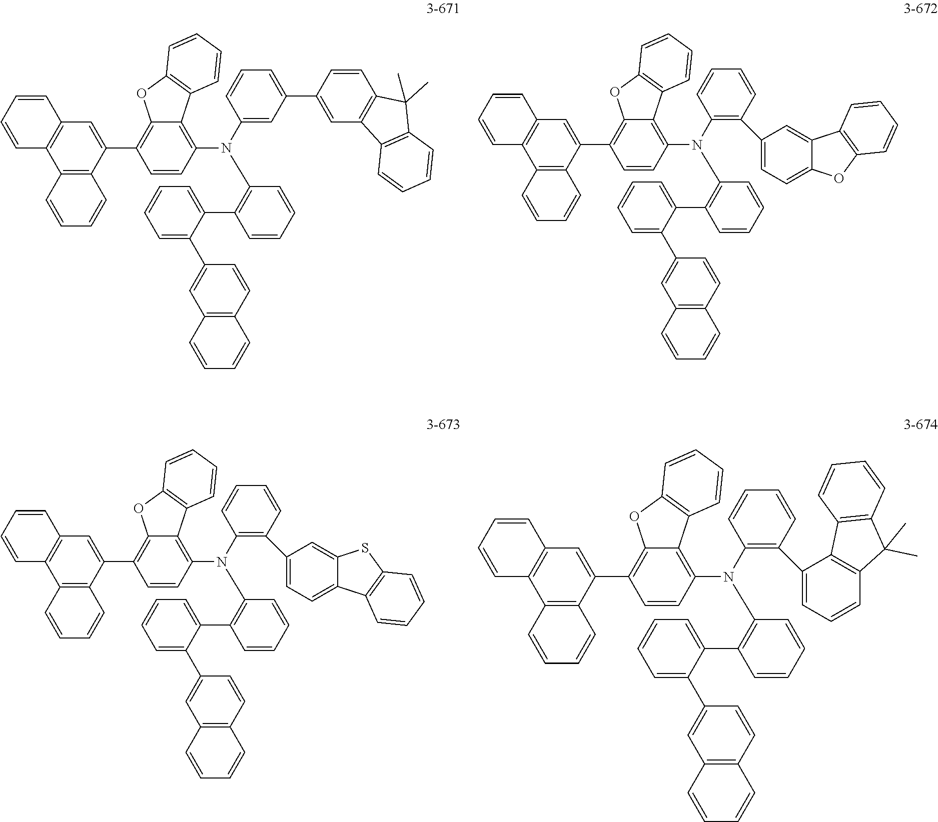 [SMILES: CC1(C)c2ccccc2-c2cc(-c3cccc(N(c4ccccc4-c4ccccc4-c4ccc5ccccc5c4)c4ccc(-c5cc6ccccc6c6ccccc56)c5oc6ccccc6c45)c3)ccc21 ; c1ccc(-c2ccccc2N(c2ccccc2-c2ccc3oc4ccccc4c3c2)c2ccc(-c3cc4ccccc4c4ccccc34)c3oc4ccccc4c23)c(-c2ccc3ccccc3c2)c1 ; c1ccc(-c2ccccc2N(c2ccccc2-c2ccc3c(c2)sc2ccccc23)c2ccc(-c3cc4ccccc4c4ccccc34)c3oc4ccccc4c23)c(-c2ccc3ccccc3c2)c1 ; CC1(C)c2ccccc2-c2c(-c3ccccc3N(c3ccccc3-c3ccccc3-c3ccc4ccccc4c3)c3ccc(-c4cc5ccccc5c5ccccc45)c4oc5ccccc5c34)cccc21]

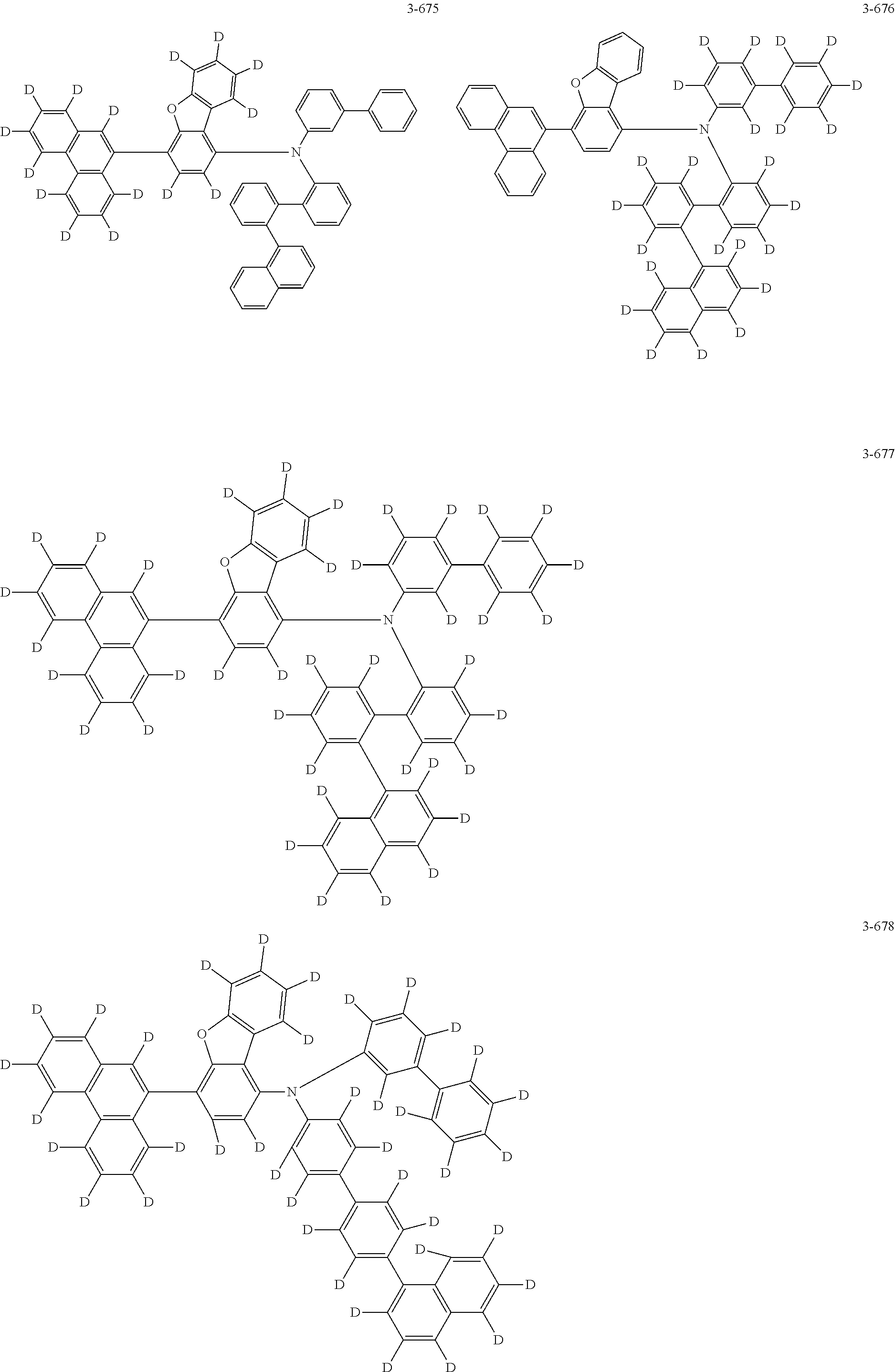

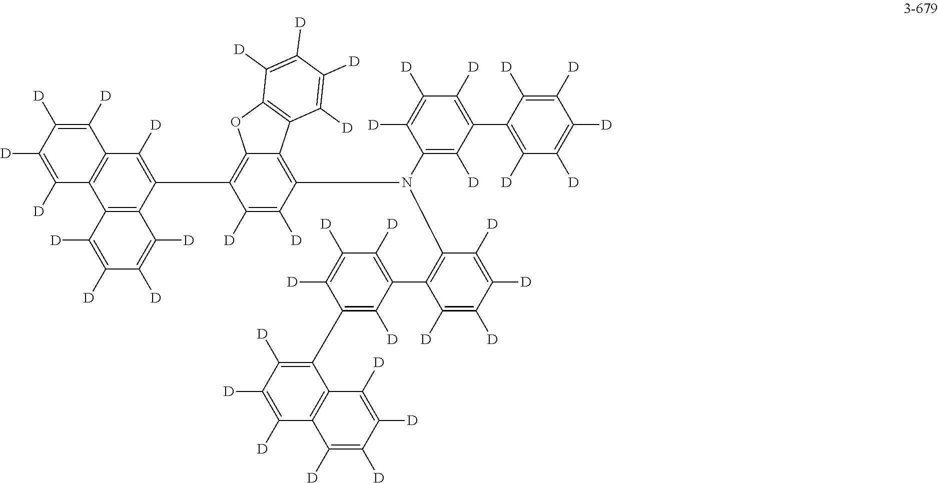 [SMILES: [2H]c1c([2H])c([2H])c(-c2c([2H])c([2H])c([2H])c(N(c3c([2H])c([2H])c([2H])c([2H])c3-c3c([2H])c([2H])c([2H])c(-c4c([2H])c([2H])c([2H])c5c([2H])c([2H])c([2H])c([2H])c45)c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c2[2H])c([2H])c1[2H]]

An organic light emitting diode according to an embodiment of the present disclosure may include a first electrode (anode), a second electrode (cathode) facing the first electrode, and one or more organic layers disposed between the first electrode and the second electrode.

At least one of the one or more organic layers may comprise an organic compound represented by Chemical Formula 1.

The organic layer may include one or more of hole injection layer (HIL), a hole transport layer (HTL), a hole transport auxiliary layer, an emitting layer (EML), an electron transport auxiliary layer, an electron transport layer (ETL), and an electron injection layer (EIL).

For example, the organic light emitting diode may have a structure in which a first electrode, a hole injection layer (HIL), a hole transport layer (HTL), a hole transport auxiliary layer, an emitting layer (EML), an electron transport layer (ETL), an electron injection layer (EIL), and a second electrode are sequentially stacked.

Here, the organic layer including the organic compound represented by Chemical Formula 1 according to an embodiment of the present disclosure may be a hole transport layer (HTL) or a hole transport auxiliary layer.

The one or more organic layers may further include one or more selected from a hole injection layer, an emitting layer, an electron transport auxiliary layer, an electron transport layer, and an electron injection layer.

For example, when the organic compound represented by Chemical Formula 1 is used as a material for the hole transport auxiliary layer, it may have an energy level suitable for serving as a hole transport auxiliary layer that transfers holes from the hole transport layer to the emitting layer and blocks electrons coming from the emitting layer.

The organic light emitting diode according to an embodiment of the present disclosure may excellently realize a target color coordinates of the emitting layer, even when a hole transport layer and/or a hole transport auxiliary layer including the organic compound represented by Chemical Formula 1 is combined with an emitting layer of any color.

The first electrode may be an anode, and may include a material having excellent transparency and conductivity, such as indium tin oxide (ITO), indium zinc oxide (IZO), tin oxide (SnO₂), or zinc oxide (ZnO).

The second electrode may be a cathode, and may include a material such as lithium (Li), aluminum (Al), aluminum-lithium (Al—Li), calcium (Ca), magnesium (Mg), magnesium-indium (Mg—In), or magnesium-silver (Mg—Ag). In addition, for a top-emission organic light emitting diode, a transparent second electrode which can transmit light may also be formed using indium tin oxide (ITO) or indium zinc oxide (IZO).

A capping layer (CPL) may be formed on the surface of the second electrode by a composition for forming a capping layer.

In addition, an encapsulation layer (or protecting layer) may be additionally disposed on the capping layer to protect the organic light emitting diode from moisture and oxygen. The encapsulation layer (or protective layer) may be formed of a curable adhesive composition containing an inorganic desiccant.

The compound for the hole injection layer or the hole transport layer is not particularly limited, and may be any compound, as long as it is conventionally used for the hole injection layer or the hole transport layer. Non-limiting examples of compounds for the hole injection layer or hole transport layer may include phthalocyanine derivatives, porphyrin derivatives, triarylamine derivatives, indolocarbazole derivatives, etc. Examples thereof may include 1,4,5,8,9,11-hexaazatriphenylene-hexacarbonitrile (HAT-CN), copper phthalocyanine (CuPc), 4,4′,4″-tris(3-methylphenylamino)triphenylamine (m-MTDATA), 4,4′,4″-tris(3-methylphenylamino)phenoxybenzene (m-MTDAPB), 4,4′,4″-tris(N-carbazolyl)triphenylamine (TCTA), 4,4′,4″-tris(N-(2-naphthyl)-N-phenylamino)triphenylamine (2-TNATA), N4,N4,N4′,N4′-tetra([1,1′-biphenyl]-4-yl)-[1,1′-biphenyl]-4,4′-diamine, bis(N-(1-naphthyl)-N-phenyl)benzidine (α-NPD), N,N′-di(naphthalen-1-yl)-N,N′-biphenyl-benzidine (NPB), N,N′-biphenyl-N,N′-bis(3-methylphenyl)-1,1′-biphenyl-4,4′-diamine (TPD), etc.

The compound included in the emitting layer is not particularly limited, and may be any compound, as long as it is conventionally used for the emitting layer. A single light emitting compound or a light emitting host compound may be used.

The light emitting compound in the emitting layer may include, but is not limited to, a compound capable of emitting light through phosphorescence, fluorescence, thermally activated delayed fluorescence, i.e., TADF (also referred to as E-type delayed fluorescence), triplet-triplet annihilation, or a combination of these processes. The light emitting compound may be selected from a variety of materials depending on the desired emission color. Non-limiting examples of the light emitting compounds include fused ring derivatives such as phenanthrene, anthracene, pyrene, tetracene, pentacene, perylene, naphthopyrene, dibenzopyrene, rubrene, and chrysene, benzoxazole derivatives, benzothiazole derivatives, benzimidazole derivatives, benzotriazole derivatives, oxazole derivatives, oxadiazole derivatives, thiazole derivatives, imidazole derivatives, thiadiazole derivatives, triazole derivatives, pyrazoline derivatives, stilbene derivatives, thiophene derivatives, tetraphenylbutadiene derivatives, cyclopentadiene derivatives, bis(styryl) derivatives, bis(styryl)arylene derivatives, diazaindacene derivatives, furan derivatives; benzofuran derivatives, isobenzofuran derivatives, dibenzofuran derivatives, coumarin derivatives, dicyanomethylenepyran derivatives, dicyanomethylenethiopyran derivatives, polymethine derivatives, cyanine derivatives, oxobenzoanthracene derivatives, xanthene derivatives, rhodamine derivatives, fluorescein derivatives, pyrylium derivatives, carbostyril derivatives, acridine derivatives, oxazine derivatives, phenylene oxide derivatives, quinacridone derivatives, quinazoline derivatives, pyrrolopyridine derivatives, furopyridine derivatives, 1,2,5-thiadiazolopyrene derivatives, pyromethene derivatives, perinone derivatives, pyrrolopyrrole derivatives, squarylium derivatives, violanthrone derivatives, phenazine derivatives, acridone derivatives, deazaflavin derivatives, fluorene derivatives, benzofluorene derivatives, aromatic boron derivatives, aromatic nitrogen boron derivatives, and metal complexes (complexes of metals such as Ir, Pt, Au, Eu, Ru, Re, Ag, and Cu with heteroaromatic ring ligands, etc.). Examples thereof include N1,N1,N6,N6-tetrakis(4-(1-silyl)phenyl)pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB), platinum octaethylporphyrin (PtOEP), Ir(ppy)₃, Ir(ppy)₂(acac), Ir(mppy)₃, Ir(PPy)₂(m-bppy), BtpIr(acac), Ir(btp)₂(acac), Ir(2-phq)₃, Hex-Ir(phq)₃, Ir(fbi)₂(acac), fac-tris(2-(3-p-xylyl)phenyl)pyridine iridium (III), Eu(dbm)₃(Phen), Ir(piq)₃, Ir(piq)₂(acac), Ir(Fliq)₂(acac), Ir(Flq)₂(acac), Ru(dtb-bpy)₃·2(PF₆), Ir(BT)₂(acac), Ir(DMP)₃, Ir(Mphq)₃, Ir(phq)₂tpy, fac-Ir(ppy)₂Pc, Ir(dp)PQ2, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, etc.

As the host compound in the emitting layer, an emissive host, a hole-transporting host, an electron-transporting host, or a combination thereof may be used. Non-limiting examples of emissive host compounds include fused ring derivatives such as anthracene or pyrene; bis(styryl) derivatives such as bis(styryl)anthracene derivatives or di(styryl)benzene derivatives; tetraphenylbutadiene derivatives; cyclopentadiene derivatives; fluorene derivatives; benzofluorene derivatives; n-phenylcarbazole (9-phenylcarbazole) derivatives; and carbazolyl nitrile derivatives. Non-limiting examples of hole-transporting host materials include carbazole derivatives, dibenzofuran derivatives, dibenzothiophene derivatives, triarylamine derivatives, indolocarbazole derivatives, and benzoxazinophenoxazine derivatives. Non-limiting examples of electron-transporting host materials include pyridine derivatives, triazine derivatives, phosphine oxide derivatives, benzofuropyridine derivatives, and dibenzooxasiline derivatives. Examples thereof include 9,10-bis(2-naphthyl)anthracene (ADN), tris(8-hydroxyquinolinato)aluminum (Alq₃), BAlq (beryllium 8-hydroxyquinolinate), DPVBi (4,4′-bis(2,2-biphenylethenyl)-1,1′-biphenyl) series, spiro-DPVBi (spiro-4,4′-bis(2,2-biphenylethenyl)-1,1′-biphenyl), LiPBO (2-(2-benzoxazolyl)phenol lithium salt), bis(biphenylvinyl)benzene, aluminum-quinoline metal complexes, and metal complexes of imidazole, thiazole, and oxazole.

The compound for the electron injection layer or the electron transport layer is not particularly limited, and may be any compound, as long as it is conventionally used for the electron injection layer or the electron transport layer. Non-limiting examples of the compound for the electron injection layer or the electron transport layer include pyridine derivatives, naphthalene derivatives, anthracene derivatives, phenanthroline derivatives, perinone derivatives, coumarin derivatives, naphthalimide derivatives, anthraquinone derivatives, diphenoquinone derivatives, diphenylquinone derivatives, perylene derivatives, oxadiazole derivatives, thiophene derivatives, triazole derivatives, thiadiazole derivatives, metal complexes of oxine derivatives, quinolinol-based metal complexes, quinoxaline derivatives and polymers thereof, benzazole compounds, gallium complexes, pyrazole derivatives, perfluorinated phenylene derivatives, triazine derivatives, pyrazine derivatives, benzoquinoline derivatives, imidazopyridine derivatives, borane derivatives, benzimidazole derivatives, benzoxazole derivatives, benzothiazole derivatives, quinoline derivatives, oligopyridine derivatives such as terpyridine, bipyridine derivatives, terpyridine derivatives, naphthyridine derivatives, aldazine derivatives, carbazole derivatives, indole derivatives, phosphine oxide derivatives, bis(styryl) derivatives, quinolinol-based metal complexes, hydroxazole-based metal complexes, azomethine-based metal complexes, tropolone-based metal complexes, flavonol-based metal complexes, benzoquinoline-based metal complexes, and metal salts. These materials may be used alone or in combination with other materials. Examples thereof include 2-(4-(9,10-di(naphthalen-2-yl)anthracen-2-yl)phenyl)-1-phenyl-1H-benzo[d]imidazole, tris(8-hydroxyquinolinato)aluminum (Alq₃), LiF, Liq, Li₂O, BaO, NaCl, and CsE The compound for the electron transport auxiliary layer, which is disposed between the electron transport layer and the emitting layer, is not particularly limited, and may be any compound, as long as it is conventionally used for the electron transport auxiliary layer. For example, the electron transport auxiliary layer may include a pyrimidine derivative, etc.

The organic light emitting diode according to an embodiment of the present disclosure may be a top-emission type or a bottom-emission type.

The organic light emitting diode according to an embodiment of the present disclosure may be applied to a display device.

The organic light emitting diode according to an embodiment of the present disclosure may be applied to transparent display devices, mobile display devices, and flexible display devices, but the present disclosure is not limited thereto.

The organic light emitting diode according to an embodiment of the present disclosure may include a tandem structure including a plurality of emitting stacks between the anode and the cathode.

Hereinafter, a representative example of the synthesis method for the above compounds will be described. However, the methods for synthesizing the compounds of the present disclosure are not limited to the method exemplified below, and the practice of the present disclosure is not limited to the following examples and experimental examples.

### Synthesis Example

The product may be synthesized as described below, but is not limited thereto.

Representative examples of synthesis of Product P1 are described, and the organic compounds represented by Chemical Formula 1 according to the present disclosure may be synthesized in a similar manner to the reaction of Product P1.

In the following reaction scheme, the solvent, catalyst, protecting group, leaving group, reaction temperature, reaction time, and equivalents of reactants, etc., are representative examples, and an equivalent solvent, catalyst, protecting group, leaving group, reaction temperature, reaction time, or equivalents of reactants, etc., may all be used.

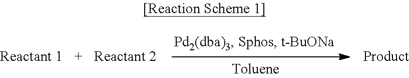

Under a nitrogen atmosphere, to a reaction flask, Reactant 1 (44 mmol), Reactant 2 (40 mmol), t-BuONa (80 mmol), Pd₂(dba)₃ (0.8 mmol), SPhos (1.6 mmol), and toluene were added to synthesize Product P1, and the mixture was stirred under reflux. After completion of the reaction, the organic layer was extracted with toluene and water. The extracted solution was dried over MgSO₄ to remove residual moisture, concentrated under reduced pressure, purified by column chromatography, and then recrystallized to obtain Product P1. The synthesis results for Product P1 are shown in Table 1 below.

The Products P2 to P101 were synthesized in the same manner as described above, except that each of the corresponding Reactant 1 and Reactant 2 for synthesizing Products P2 to P101 was used instead of the Reactant 1 and Reactant 2 used for synthesizing Product P1 in the above synthesis method. The compound structures of Products P1 to P101, the respective structures of Reactant 1 and Reactant 2 for synthesizing them, and the synthesis results, are shown in Table 1 below.

TABLE 1

| Item | Reactant 1 | Reactant 2 | |
|---|---|---|---|
| P1 | 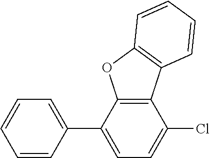 [SMILES: Clc1ccc(-c2ccccc2)c2oc3ccccc3c12] | 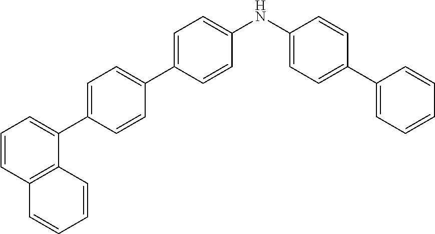 [SMILES: c1ccc(-c2ccc(Nc3ccc(-c4ccc(-c5cccc6ccccc56)cc4)cc3)cc2)cc1] | |
| P2 | 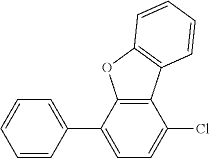 [SMILES: Clc1ccc(-c2ccccc2)c2oc3ccccc3c12] | 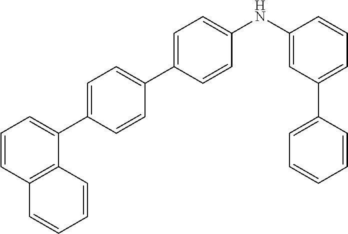 [SMILES: c1ccc(-c2cccc(Nc3ccc(-c4ccc(-c5cccc6ccccc56)cc4)cc3)c2)cc1] | |
| P3 | 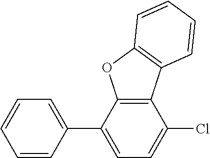 [SMILES: Clc1ccc(-c2ccccc2)c2oc3ccccc3c12] | 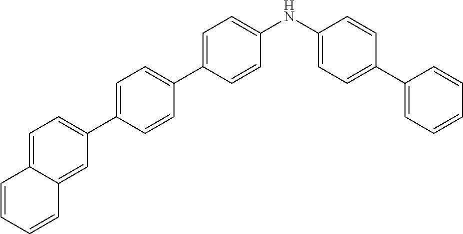 [SMILES: c1ccc(-c2ccc(Nc3ccc(-c4ccc(-c5ccc6ccccc6c5)cc4)cc3)cc2)cc1] | |
| P4 | 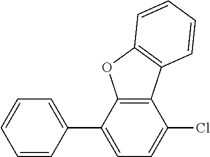 [SMILES: Clc1ccc(-c2ccccc2)c2oc3ccccc3c12] | 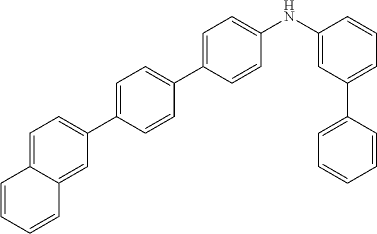 [SMILES: c1ccc(-c2cccc(Nc3ccc(-c4ccc(-c5ccc6ccccc6c5)cc4)cc3)c2)cc1] | |
| P5 | 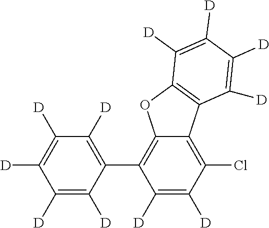 [SMILES: [2H]c1c([2H])c([2H])c(-c2c([2H])c([2H])c(Cl)c3c2oc2c([2H])c([2H])c([2H])c([2H])c23)c([2H])c1[2H]] | 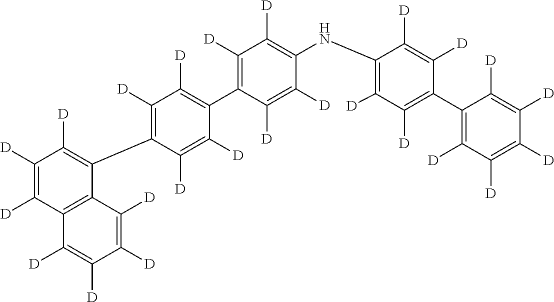 [SMILES: [2H]c1c([2H])c([2H])c(-c2c([2H])c([2H])c(Nc3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c([2H])c2[2H])c([2H])c1[2H]] | |
| P6 | 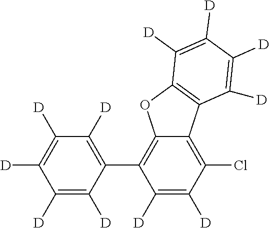 [SMILES: [2H]c1c([2H])c([2H])c(-c2c([2H])c([2H])c(Cl)c3c2oc2c([2H])c([2H])c([2H])c([2H])c23)c([2H])c1[2H]] | 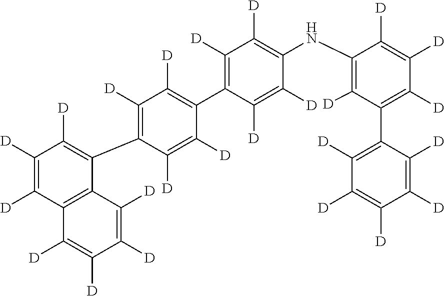 [SMILES: [2H]c1c([2H])c([2H])c(-c2c([2H])c([2H])c([2H])c(Nc3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c2[2H])c([2H])c1[2H]] | |
| P7 | 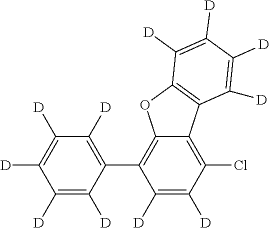 [SMILES: [2H]c1c([2H])c([2H])c(-c2c([2H])c([2H])c(Cl)c3c2oc2c([2H])c([2H])c([2H])c([2H])c23)c([2H])c1[2H]] | 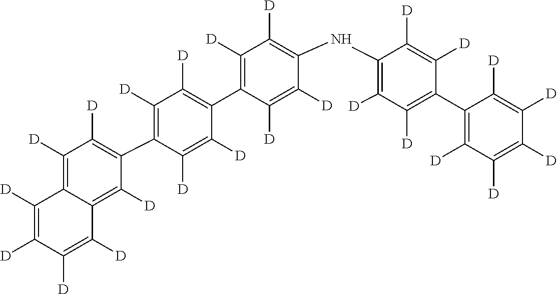 [SMILES: [2H]c1c([2H])c([2H])c(-c2c([2H])c([2H])c(Nc3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c([2H])c2[2H])c([2H])c1[2H]] | |
| P8 | 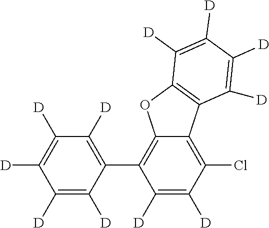 [SMILES: [2H]c1c([2H])c([2H])c(-c2c([2H])c([2H])c(Cl)c3c2oc2c([2H])c([2H])c([2H])c([2H])c23)c([2H])c1[2H]] | 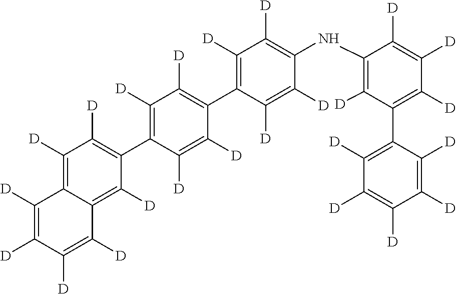 [SMILES: [2H]c1c([2H])c([2H])c(-c2c([2H])c([2H])c([2H])c(Nc3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c2[2H])c([2H])c1[2H]] | |
| P9 | 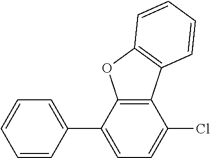 [SMILES: Clc1ccc(-c2ccccc2)c2oc3ccccc3c12] | 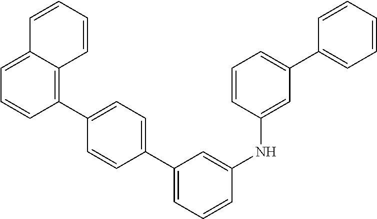 [SMILES: c1ccc(-c2cccc(Nc3cccc(-c4ccc(-c5cccc6ccccc56)cc4)c3)c2)cc1] | |
| P10 | 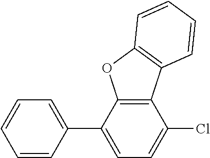 [SMILES: Clc1ccc(-c2ccccc2)c2oc3ccccc3c12] | 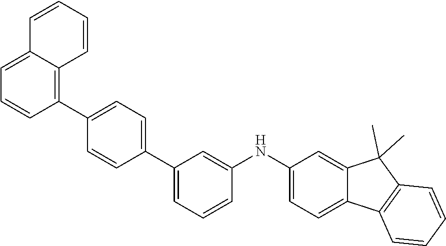 [SMILES: CC1(C)c2ccccc2-c2ccc(Nc3cccc(-c4ccc(-c5cccc6ccccc56)cc4)c3)cc21] | |
| P11 | 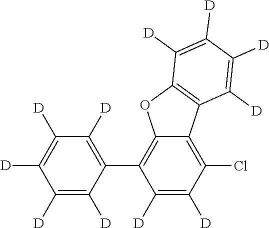 [SMILES: [2H]c1c([2H])c([2H])c(-c2c([2H])c([2H])c(Cl)c3c2oc2c([2H])c([2H])c([2H])c([2H])c23)c([2H])c1[2H]] | 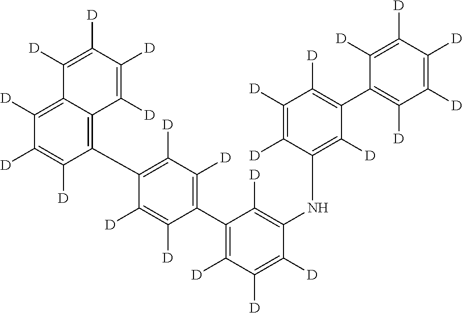 [SMILES: [2H]c1c([2H])c([2H])c(-c2c([2H])c([2H])c([2H])c(Nc3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])c2[2H])c([2H])c1[2H]] | |
| P12 | 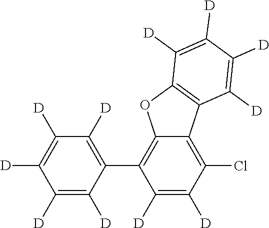 [SMILES: [2H]c1c([2H])c([2H])c(-c2c([2H])c([2H])c(Cl)c3c2oc2c([2H])c([2H])c([2H])c([2H])c23)c([2H])c1[2H]] | 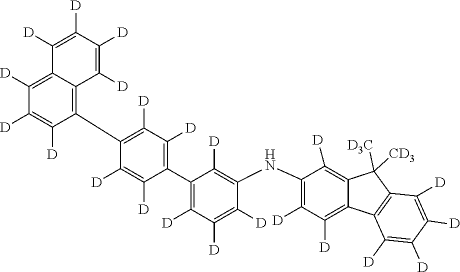 [SMILES: [2H]c1c([2H])c(Nc2c([2H])c([2H])c3c(c2[2H])C(C([2H])([2H])[2H])(C([2H])([2H])[2H])c2c([2H])c([2H])c([2H])c([2H])c2-3)c([2H])c(-c2c([2H])c([2H])c(-c3c([2H])c([2H])c([2H])c4c([2H])c([2H])c([2H])c([2H])c34)c([2H])c2[2H])c1[2H]] | |
| P13 | 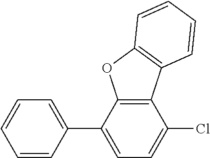 [SMILES: Clc1ccc(-c2ccccc2)c2oc3ccccc3c12] | 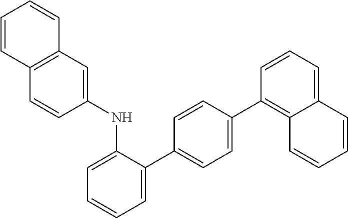 [SMILES: c1ccc(-c2ccc(-c3cccc4ccccc34)cc2)c(Nc2ccc3ccccc3c2)c1] | |
| P14 | 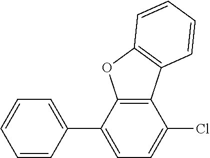 [SMILES: Clc1ccc(-c2ccccc2)c2oc3ccccc3c12] | 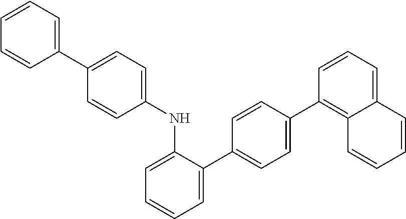 [SMILES: c1ccc(-c2ccc(Nc3ccccc3-c3ccc(-c4cccc5ccccc45)cc3)cc2)cc1] | |
| P15 | 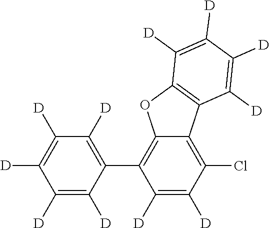 [SMILES: [2H]c1c([2H])c([2H])c(-c2c([2H])c([2H])c(Cl)c3c2oc2c([2H])c([2H])c([2H])c([2H])c23)c([2H])c1[2H]] | 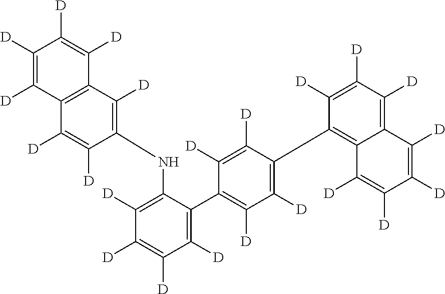 [SMILES: [2H]c1c([2H])c([2H])c(-c2c([2H])c([2H])c(-c3c([2H])c([2H])c([2H])c4c([2H])c([2H])c([2H])c([2H])c34)c([2H])c2[2H])c(Nc2c([2H])c([2H])c3c([2H])c([2H])c([2H])c([2H])c3c2[2H])c1[2H]] | |
| P16 | 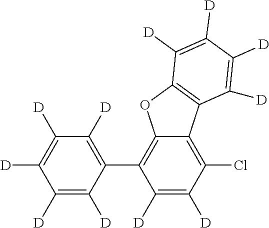 [SMILES: [2H]c1c([2H])c([2H])c(-c2c([2H])c([2H])c(Cl)c3c2oc2c([2H])c([2H])c([2H])c([2H])c23)c([2H])c1[2H]] | 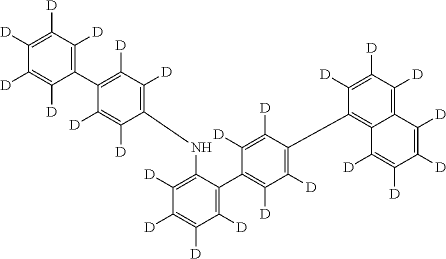 [SMILES: [2H]c1c([2H])c([2H])c(-c2c([2H])c([2H])c(Nc3c([2H])c([2H])c([2H])c([2H])c3-c3c([2H])c([2H])c(-c4c([2H])c([2H])c([2H])c5c([2H])c([2H])c([2H])c([2H])c45)c([2H])c3[2H])c([2H])c2[2H])c([2H])c1[2H]] | |
| P17 | 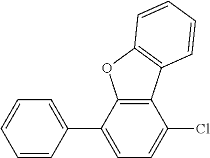 [SMILES: Clc1ccc(-c2ccccc2)c2oc3ccccc3c12] | 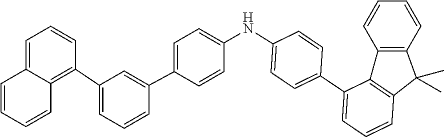 [SMILES: CC1(C)c2ccccc2-c2c(-c3ccc(Nc4ccc(-c5cccc(-c6cccc7ccccc67)c5)cc4)cc3)cccc21] | |
| P18 | 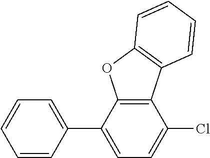 [SMILES: Clc1ccc(-c2ccccc2)c2oc3ccccc3c12] | 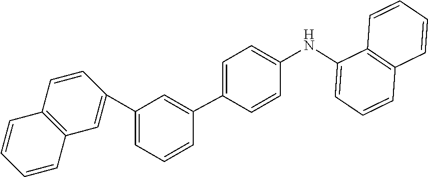 [SMILES: c1cc(-c2ccc(Nc3cccc4ccccc34)cc2)cc(-c2ccc3ccccc3c2)c1] | |
| P19 | 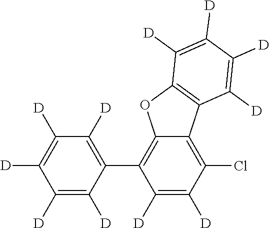 [SMILES: [2H]c1c([2H])c([2H])c(-c2c([2H])c([2H])c(Cl)c3c2oc2c([2H])c([2H])c([2H])c([2H])c23)c([2H])c1[2H]] | 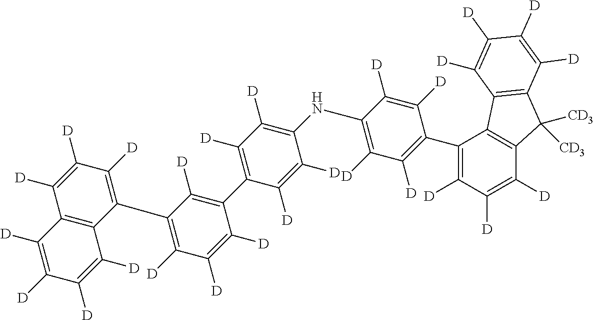 [SMILES: [2H]c1c([2H])c(-c2c([2H])c([2H])c(Nc3c([2H])c([2H])c(-c4c([2H])c([2H])c([2H])c5c4-c4c([2H])c([2H])c([2H])c([2H])c4C5(C([2H])([2H])[2H])C([2H])([2H])[2H])c([2H])c3[2H])c([2H])c2[2H])c([2H])c(-c2c([2H])c([2H])c([2H])c3c([2H])c([2H])c([2H])c([2H])c23)c1[2H]] | |
| P20 | 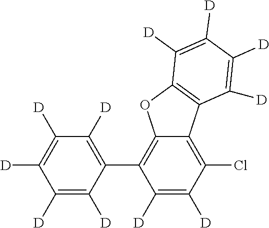 [SMILES: [2H]c1c([2H])c([2H])c(-c2c([2H])c([2H])c(Cl)c3c2oc2c([2H])c([2H])c([2H])c([2H])c23)c([2H])c1[2H]] | 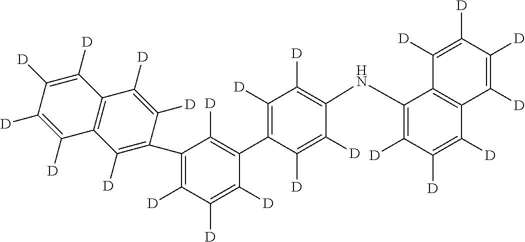 [SMILES: [2H]c1c([2H])c(-c2c([2H])c([2H])c(Nc3c([2H])c([2H])c([2H])c4c([2H])c([2H])c([2H])c([2H])c34)c([2H])c2[2H])c([2H])c(-c2c([2H])c([2H])c3c([2H])c([2H])c([2H])c([2H])c3c2[2H])c1[2H]] | |
| P21 | 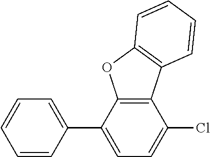 [SMILES: Clc1ccc(-c2ccccc2)c2oc3ccccc3c12] | 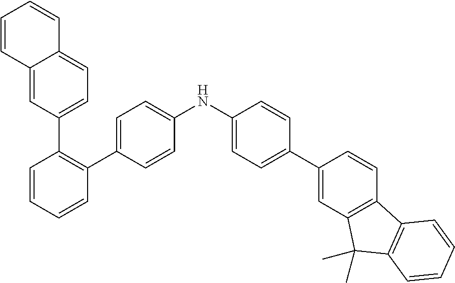 [SMILES: CC1(C)c2ccccc2-c2ccc(-c3ccc(Nc4ccc(-c5ccccc5-c5ccc6ccccc6c5)cc4)cc3)cc21] | |
| P22 | 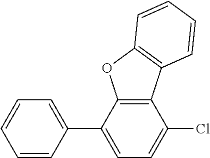 [SMILES: Clc1ccc(-c2ccccc2)c2oc3ccccc3c12] | 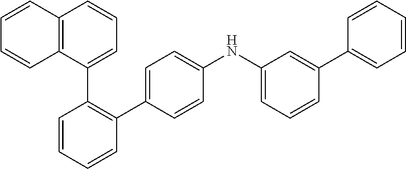 [SMILES: c1ccc(-c2cccc(Nc3ccc(-c4ccccc4-c4cccc5ccccc45)cc3)c2)cc1] | |
| P23 | 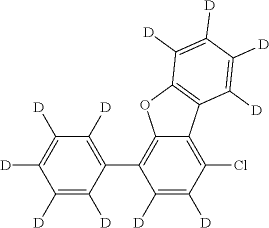 [SMILES: [2H]c1c([2H])c([2H])c(-c2c([2H])c([2H])c(Cl)c3c2oc2c([2H])c([2H])c([2H])c([2H])c23)c([2H])c1[2H]] | 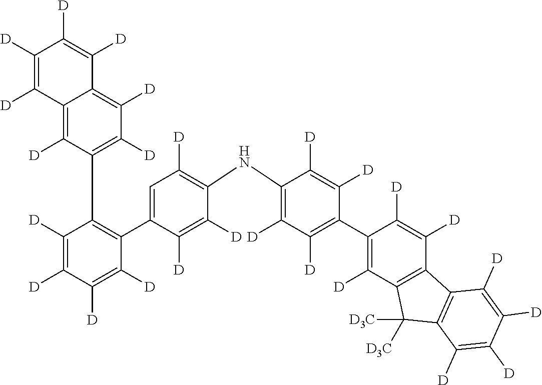 [SMILES: [2H]c1cc(-c2c([2H])c([2H])c([2H])c([2H])c2-c2c([2H])c([2H])c3c([2H])c([2H])c([2H])c([2H])c3c2[2H])c([2H])c([2H])c1Nc1c([2H])c([2H])c(-c2c([2H])c([2H])c3c(c2[2H])C(C([2H])([2H])[2H])(C([2H])([2H])[2H])c2c([2H])c([2H])c([2H])c([2H])c2-3)c([2H])c1[2H]] | |
| P24 | 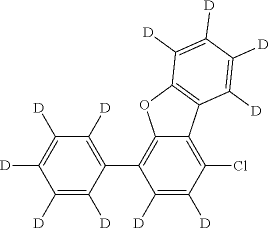 [SMILES: [2H]c1c([2H])c([2H])c(-c2c([2H])c([2H])c(Cl)c3c2oc2c([2H])c([2H])c([2H])c([2H])c23)c([2H])c1[2H]] | 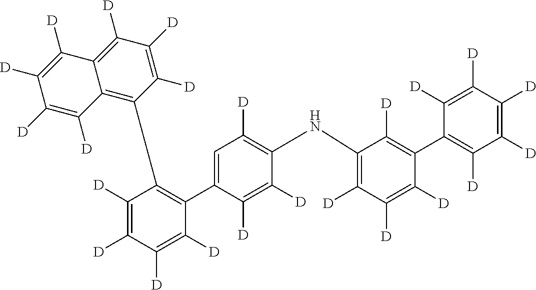 [SMILES: [2H]c1cc(-c2c([2H])c([2H])c([2H])c([2H])c2-c2c([2H])c([2H])c([2H])c3c([2H])c([2H])c([2H])c([2H])c23)c([2H])c([2H])c1Nc1c([2H])c([2H])c([2H])c(-c2c([2H])c([2H])c([2H])c([2H])c2[2H])c1[2H]] | |
| P25 | 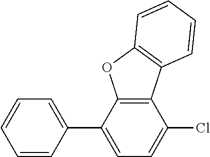 [SMILES: Clc1ccc(-c2ccccc2)c2oc3ccccc3c12] | 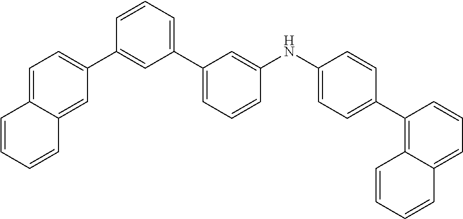 [SMILES: c1cc(Nc2ccc(-c3cccc4ccccc34)cc2)cc(-c2cccc(-c3ccc4ccccc4c3)c2)c1] | |
| P26 | 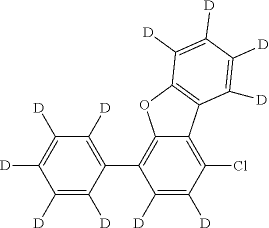 [SMILES: [2H]c1c([2H])c([2H])c(-c2c([2H])c([2H])c(Cl)c3c2oc2c([2H])c([2H])c([2H])c([2H])c23)c([2H])c1[2H]] | 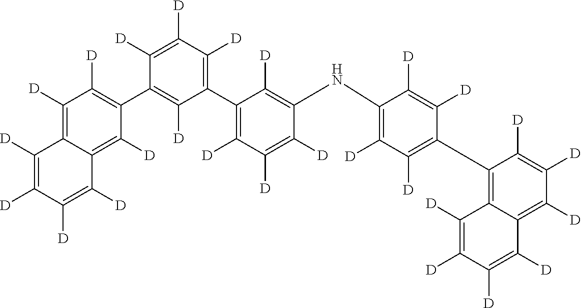 [SMILES: [2H]c1c([2H])c(Nc2c([2H])c([2H])c(-c3c([2H])c([2H])c([2H])c4c([2H])c([2H])c([2H])c([2H])c34)c([2H])c2[2H])c([2H])c(-c2c([2H])c([2H])c([2H])c(-c3c([2H])c([2H])c4c([2H])c([2H])c([2H])c([2H])c4c3[2H])c2[2H])c1[2H]] | |
| P27 | 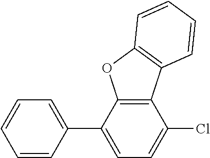 [SMILES: Clc1ccc(-c2ccccc2)c2oc3ccccc3c12] | 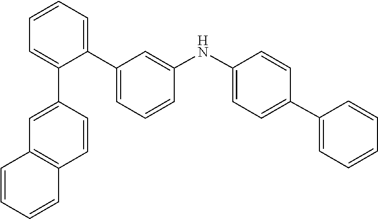 [SMILES: c1ccc(-c2ccc(Nc3cccc(-c4ccccc4-c4ccc5ccccc5c4)c3)cc2)cc1] | |
| P28 | 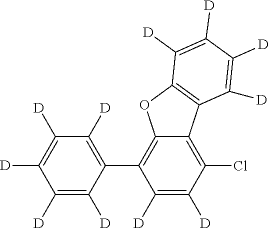 [SMILES: [2H]c1c([2H])c([2H])c(-c2c([2H])c([2H])c(Cl)c3c2oc2c([2H])c([2H])c([2H])c([2H])c23)c([2H])c1[2H]] | 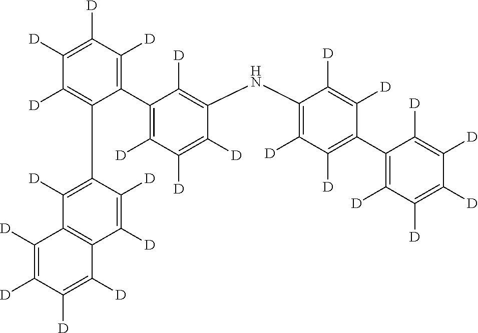 [SMILES: [2H]c1c([2H])c([2H])c(-c2c([2H])c([2H])c(Nc3c([2H])c([2H])c([2H])c(-c4c([2H])c([2H])c([2H])c([2H])c4-c4c([2H])c([2H])c5c([2H])c([2H])c([2H])c([2H])c5c4[2H])c3[2H])c([2H])c2[2H])c([2H])c1[2H]] | |
| P29 | 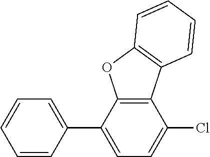 [SMILES: Clc1ccc(-c2ccccc2)c2oc3ccccc3c12] | 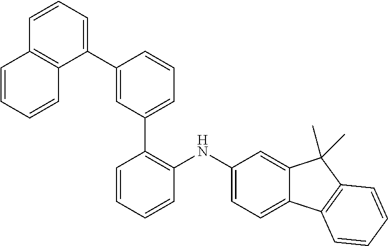 [SMILES: CC1(C)c2ccccc2-c2ccc(Nc3ccccc3-c3cccc(-c4cccc5ccccc45)c3)cc21] | |
| P30 | 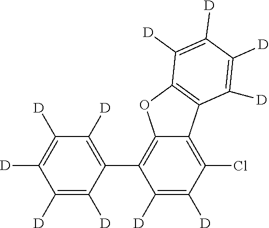 [SMILES: [2H]c1c([2H])c([2H])c(-c2c([2H])c([2H])c(Cl)c3c2oc2c([2H])c([2H])c([2H])c([2H])c23)c([2H])c1[2H]] | 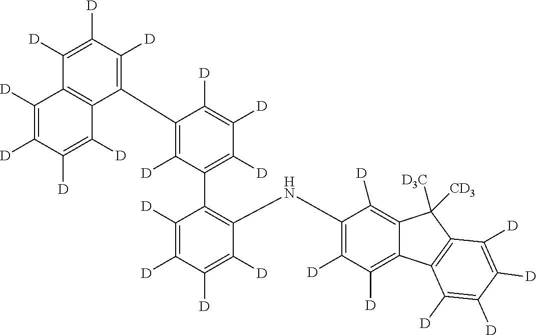 [SMILES: [2H]c1c([2H])c([2H])c(-c2c([2H])c([2H])c([2H])c(-c3c([2H])c([2H])c([2H])c4c([2H])c([2H])c([2H])c([2H])c34)c2[2H])c(Nc2c([2H])c([2H])c3c(c2[2H])C(C([2H])([2H])[2H])(C([2H])([2H])[2H])c2c([2H])c([2H])c([2H])c([2H])c2-3)c1[2H]] | |
| P31 | 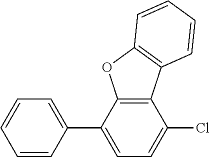 [SMILES: Clc1ccc(-c2ccccc2)c2oc3ccccc3c12] | 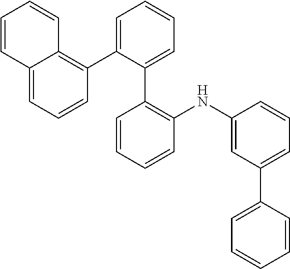 [SMILES: c1ccc(-c2cccc(Nc3ccccc3-c3ccccc3-c3cccc4ccccc34)c2)cc1] | |
| P32 | 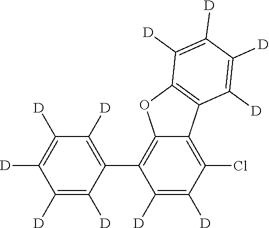 [SMILES: [2H]c1c([2H])c([2H])c(-c2c([2H])c([2H])c(Cl)c3c2oc2c([2H])c([2H])c([2H])c([2H])c23)c([2H])c1[2H]] | 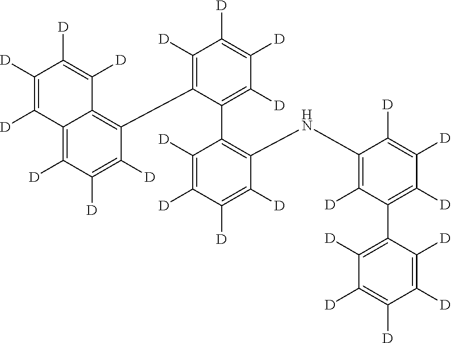 [SMILES: [2H]c1c([2H])c([2H])c(-c2c([2H])c([2H])c([2H])c(Nc3c([2H])c([2H])c([2H])c([2H])c3-c3c([2H])c([2H])c([2H])c([2H])c3-c3c([2H])c([2H])c([2H])c4c([2H])c([2H])c([2H])c([2H])c34)c2[2H])c([2H])c1[2H]] | |
| P33 | 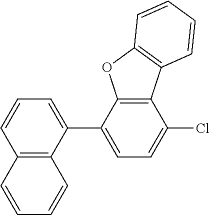 [SMILES: Clc1ccc(-c2cccc3ccccc23)c2oc3ccccc3c12] | 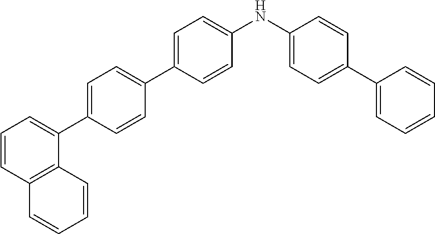 [SMILES: c1ccc(-c2ccc(Nc3ccc(-c4ccc(-c5cccc6ccccc56)cc4)cc3)cc2)cc1] | |
| P34 | 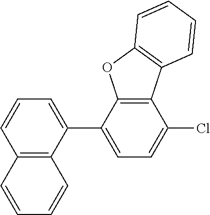 [SMILES: Clc1ccc(-c2cccc3ccccc23)c2oc3ccccc3c12] | 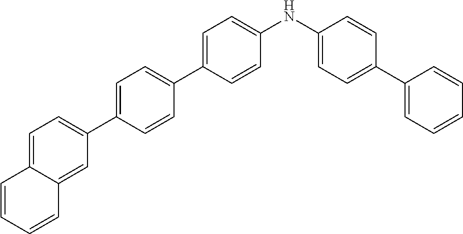 [SMILES: c1ccc(-c2ccc(Nc3ccc(-c4ccc(-c5ccc6ccccc6c5)cc4)cc3)cc2)cc1] | |
| P35 | 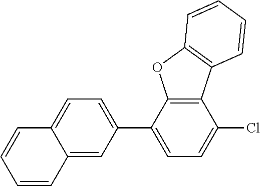 [SMILES: Clc1ccc(-c2ccc3ccccc3c2)c2oc3ccccc3c12] | 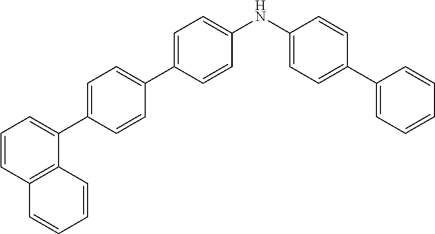 [SMILES: c1ccc(-c2ccc(Nc3ccc(-c4ccc(-c5cccc6ccccc56)cc4)cc3)cc2)cc1] | |
| P36 | 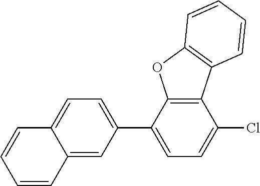 [SMILES: Clc1ccc(-c2ccc3ccccc3c2)c2oc3ccccc3c12] | 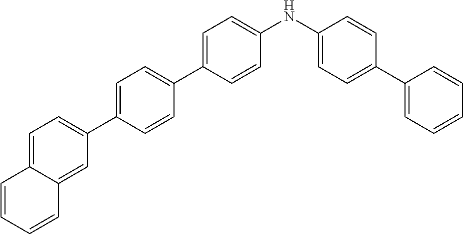 [SMILES: c1ccc(-c2ccc(Nc3ccc(-c4ccc(-c5ccc6ccccc6c5)cc4)cc3)cc2)cc1] | |
| P37 | 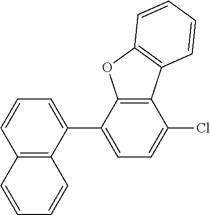 [SMILES: Clc1ccc(-c2cccc3ccccc23)c2oc3ccccc3c12] | 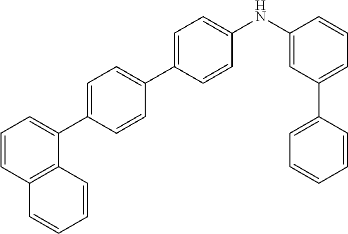 [SMILES: c1ccc(-c2cccc(Nc3ccc(-c4ccc(-c5cccc6ccccc56)cc4)cc3)c2)cc1] | |
| P38 | 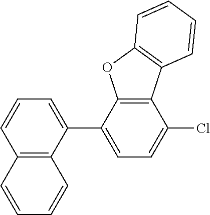 [SMILES: Clc1ccc(-c2cccc3ccccc23)c2oc3ccccc3c12] | 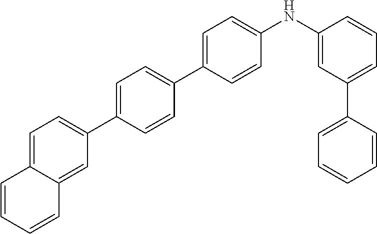 [SMILES: c1ccc(-c2cccc(Nc3ccc(-c4ccc(-c5ccc6ccccc6c5)cc4)cc3)c2)cc1] | |
| P39 | 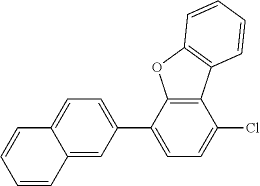 [SMILES: Clc1ccc(-c2ccc3ccccc3c2)c2oc3ccccc3c12] | 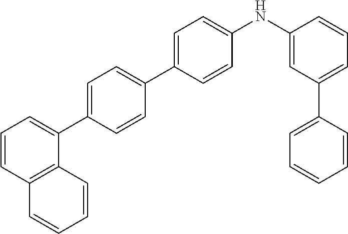 [SMILES: c1ccc(-c2cccc(Nc3ccc(-c4ccc(-c5cccc6ccccc56)cc4)cc3)c2)cc1] | |
| P40 | 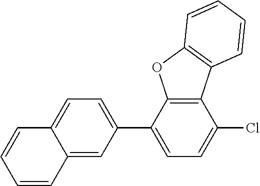 [SMILES: Clc1ccc(-c2ccc3ccccc3c2)c2oc3ccccc3c12] | 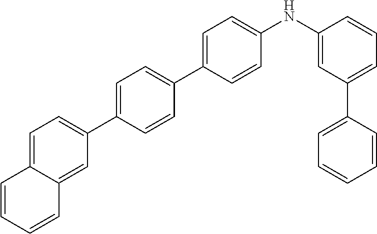 [SMILES: c1ccc(-c2cccc(Nc3ccc(-c4ccc(-c5ccc6ccccc6c5)cc4)cc3)c2)cc1] | |
| P41 | 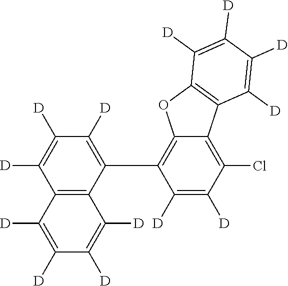 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 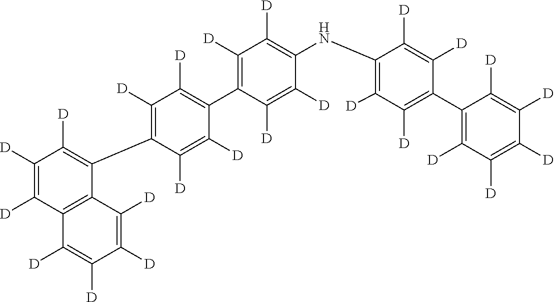 [SMILES: [2H]c1c([2H])c([2H])c(-c2c([2H])c([2H])c(Nc3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c([2H])c2[2H])c([2H])c1[2H]] | |
| P42 | 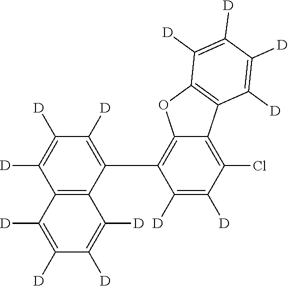 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 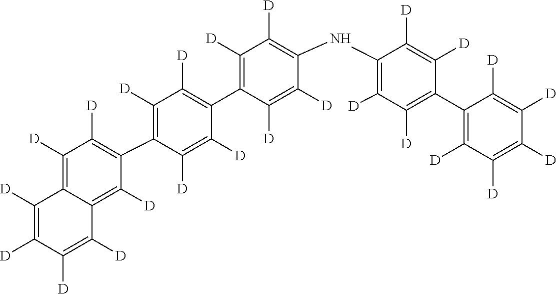 [SMILES: [2H]c1c([2H])c([2H])c(-c2c([2H])c([2H])c(Nc3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c([2H])c2[2H])c([2H])c1[2H]] | |
| P43 | 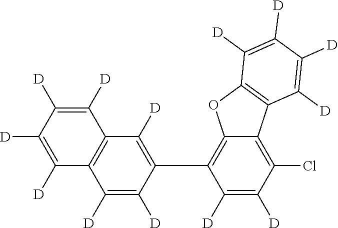 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 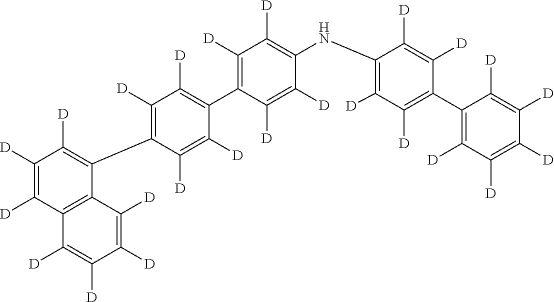 [SMILES: [2H]c1c([2H])c([2H])c(-c2c([2H])c([2H])c(Nc3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c([2H])c2[2H])c([2H])c1[2H]] | |
| P44 | 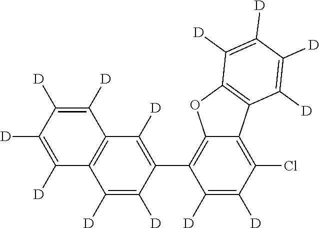 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 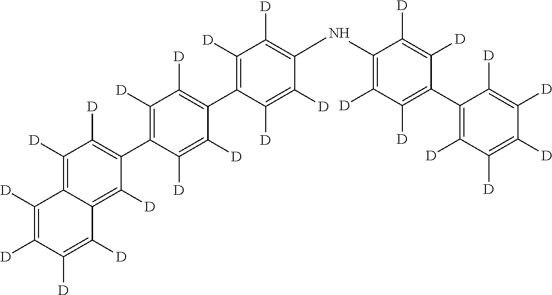 [SMILES: [2H]c1c([2H])c([2H])c(-c2c([2H])c([2H])c(Nc3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c([2H])c2[2H])c([2H])c1[2H]] | |
| P45 | 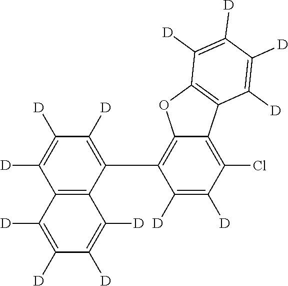 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 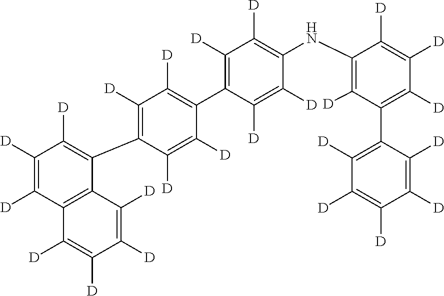 [SMILES: [2H]c1c([2H])c([2H])c(-c2c([2H])c([2H])c([2H])c(Nc3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c2[2H])c([2H])c1[2H]] | |
| P46 | 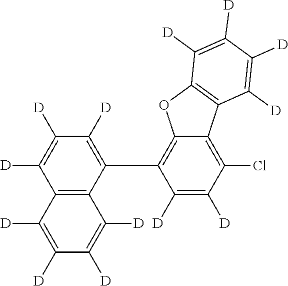 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 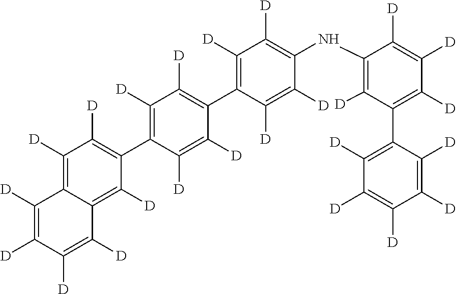 [SMILES: [2H]c1c([2H])c([2H])c(-c2c([2H])c([2H])c([2H])c(Nc3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c2[2H])c([2H])c1[2H]] | |
| P47 | 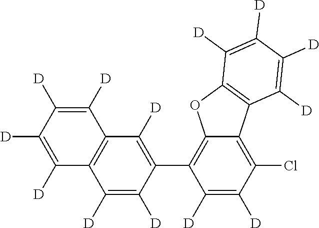 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 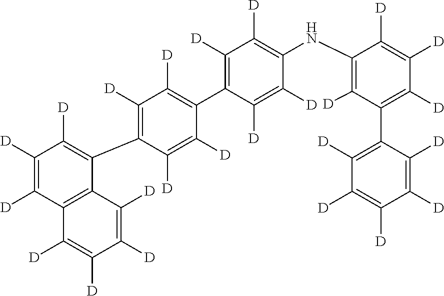 [SMILES: [2H]c1c([2H])c([2H])c(-c2c([2H])c([2H])c([2H])c(Nc3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c2[2H])c([2H])c1[2H]] | |
| P48 | 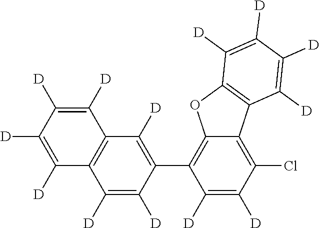 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 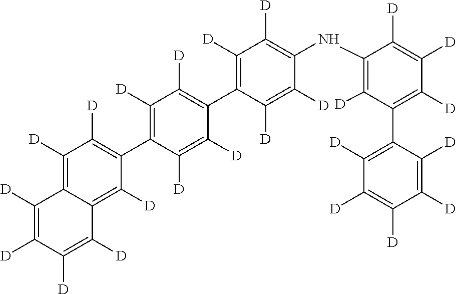 [SMILES: [2H]c1c([2H])c([2H])c(-c2c([2H])c([2H])c([2H])c(Nc3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c2[2H])c([2H])c1[2H]] | |
| P49 | 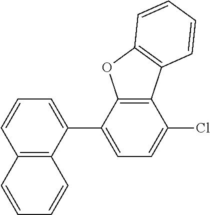 [SMILES: Clc1ccc(-c2cccc3ccccc23)c2oc3ccccc3c12] | 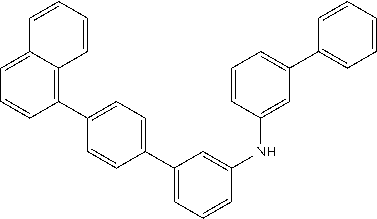 [SMILES: c1ccc(-c2cccc(Nc3cccc(-c4ccc(-c5cccc6ccccc56)cc4)c3)c2)cc1] | |
| P50 | 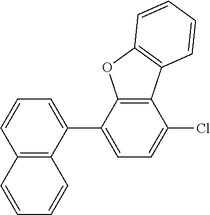 [SMILES: Clc1ccc(-c2cccc3ccccc23)c2oc3ccccc3c12] | 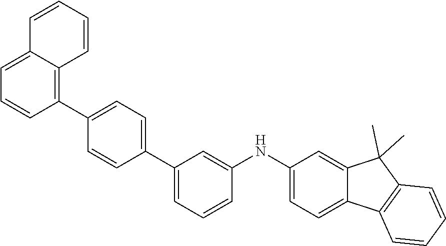 [SMILES: CC1(C)c2ccccc2-c2ccc(Nc3cccc(-c4ccc(-c5cccc6ccccc56)cc4)c3)cc21] | |
| P51 | 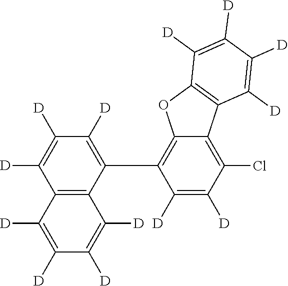 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 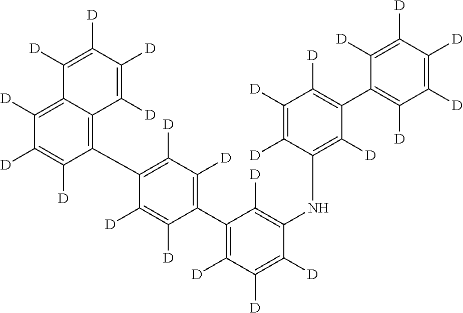 [SMILES: [2H]c1c([2H])c([2H])c(-c2c([2H])c([2H])c([2H])c(Nc3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])c2[2H])c([2H])c1[2H]] | |
| P52 | 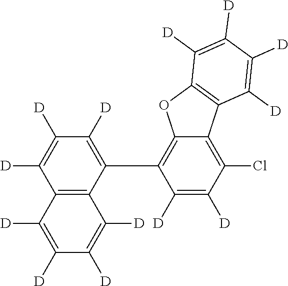 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 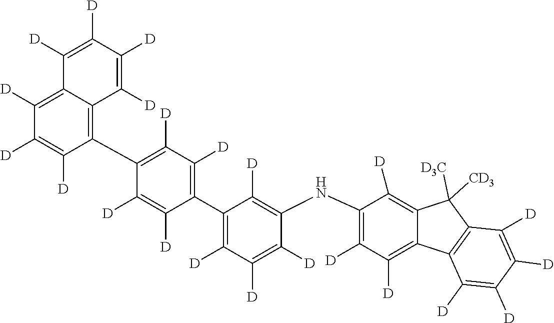 [SMILES: [2H]c1c([2H])c(Nc2c([2H])c([2H])c3c(c2[2H])C(C([2H])([2H])[2H])(C([2H])([2H])[2H])c2c([2H])c([2H])c([2H])c([2H])c2-3)c([2H])c(-c2c([2H])c([2H])c(-c3c([2H])c([2H])c([2H])c4c([2H])c([2H])c([2H])c([2H])c34)c([2H])c2[2H])c1[2H]] | |
| P53 | 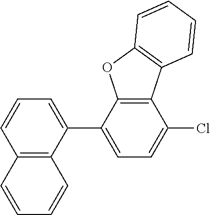 [SMILES: Clc1ccc(-c2cccc3ccccc23)c2oc3ccccc3c12] | 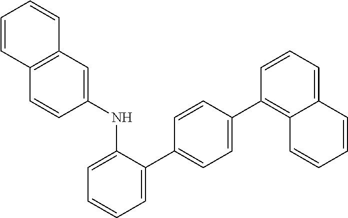 [SMILES: c1ccc(-c2ccc(-c3cccc4ccccc34)cc2)c(Nc2ccc3ccccc3c2)c1] | |
| P54 | 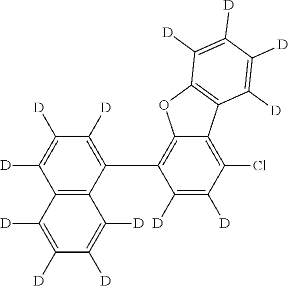 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 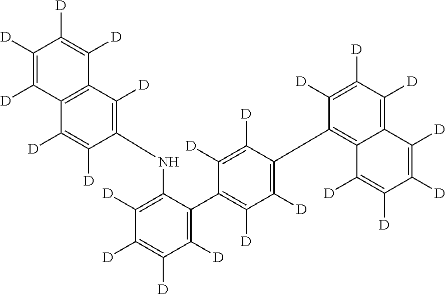 [SMILES: [2H]c1c([2H])c([2H])c(-c2c([2H])c([2H])c(-c3c([2H])c([2H])c([2H])c4c([2H])c([2H])c([2H])c([2H])c34)c([2H])c2[2H])c(Nc2c([2H])c([2H])c3c([2H])c([2H])c([2H])c([2H])c3c2[2H])c1[2H]] | |
| P55 | 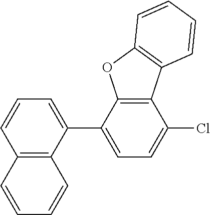 [SMILES: Clc1ccc(-c2cccc3ccccc23)c2oc3ccccc3c12] | 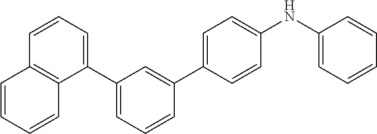 [SMILES: c1ccc(Nc2ccc(-c3cccc(-c4cccc5ccccc45)c3)cc2)cc1] | |
| P56 | 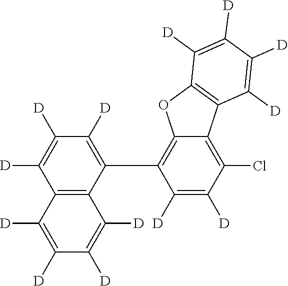 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 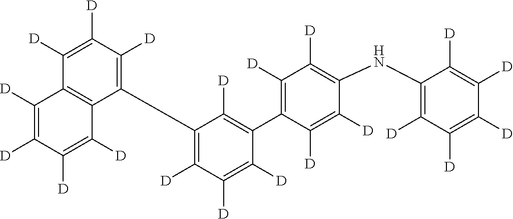 [SMILES: [2H]c1c([2H])c([2H])c(Nc2c([2H])c([2H])c(-c3c([2H])c([2H])c([2H])c(-c4c([2H])c([2H])c([2H])c5c([2H])c([2H])c([2H])c([2H])c45)c3[2H])c([2H])c2[2H])c([2H])c1[2H]] | |
| P57 | 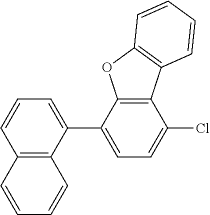 [SMILES: Clc1ccc(-c2cccc3ccccc23)c2oc3ccccc3c12] | 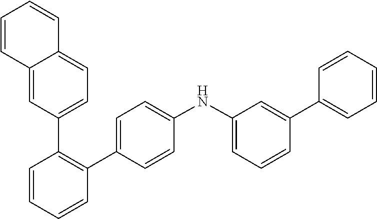 [SMILES: c1ccc(-c2cccc(Nc3ccc(-c4ccccc4-c4ccc5ccccc5c4)cc3)c2)cc1] | |
| P58 | 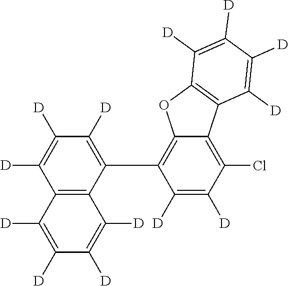 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 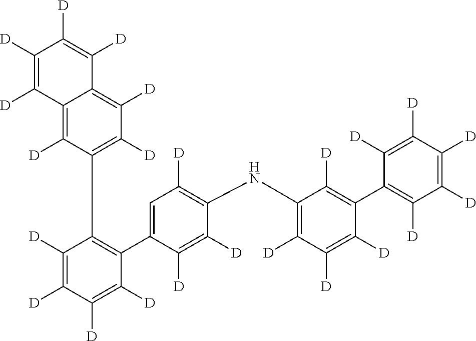 [SMILES: [2H]c1cc(-c2c([2H])c([2H])c([2H])c([2H])c2-c2c([2H])c([2H])c3c([2H])c([2H])c([2H])c([2H])c3c2[2H])c([2H])c([2H])c1Nc1c([2H])c([2H])c([2H])c(-c2c([2H])c([2H])c([2H])c([2H])c2[2H])c1[2H]] | |
| P59 | 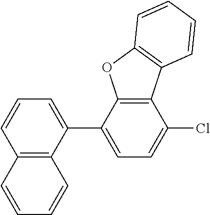 [SMILES: Clc1ccc(-c2cccc3ccccc23)c2oc3ccccc3c12] | 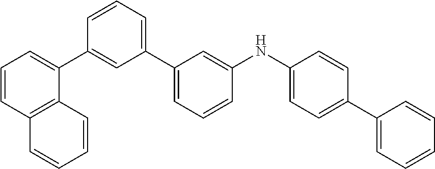 [SMILES: c1ccc(-c2ccc(Nc3cccc(-c4cccc(-c5cccc6ccccc56)c4)c3)cc2)cc1] | |
| P60 | 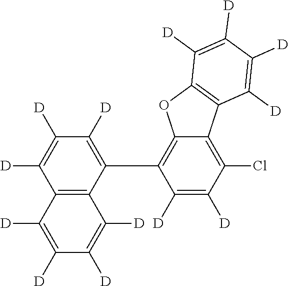 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 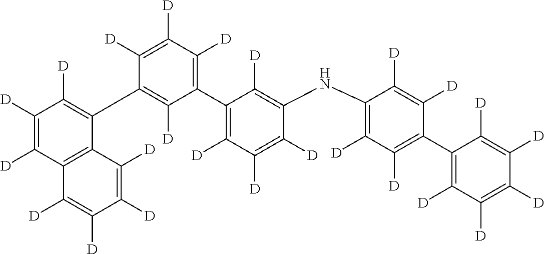 [SMILES: [2H]c1c([2H])c([2H])c(-c2c([2H])c([2H])c(Nc3c([2H])c([2H])c([2H])c(-c4c([2H])c([2H])c([2H])c(-c5c([2H])c([2H])c([2H])c6c([2H])c([2H])c([2H])c([2H])c56)c4[2H])c3[2H])c([2H])c2[2H])c([2H])c1[2H]] | |
| P61 | 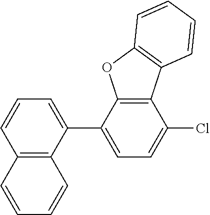 [SMILES: Clc1ccc(-c2cccc3ccccc23)c2oc3ccccc3c12] | 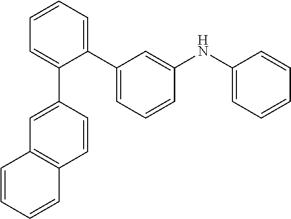 [SMILES: c1ccc(Nc2cccc(-c3ccccc3-c3ccc4ccccc4c3)c2)cc1] | |
| P62 | 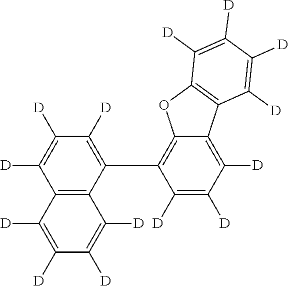 [SMILES: [2H]c1c([2H])c([2H])c2c(oc3c(-c4c([2H])c([2H])c([2H])c5c([2H])c([2H])c([2H])c([2H])c45)c([2H])c([2H])c([2H])c32)c1[2H]] | 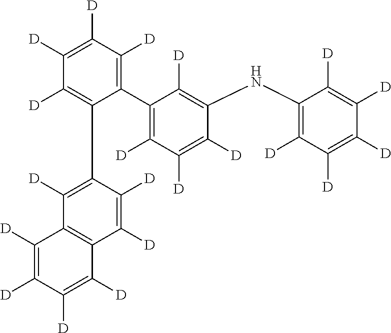 [SMILES: [2H]c1c([2H])c([2H])c(Nc2c([2H])c([2H])c([2H])c(-c3c([2H])c([2H])c([2H])c([2H])c3-c3c([2H])c([2H])c4c([2H])c([2H])c([2H])c([2H])c4c3[2H])c2[2H])c([2H])c1[2H]] | |
| P63 | 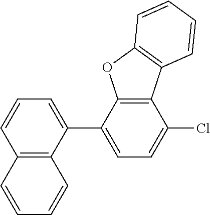 [SMILES: Clc1ccc(-c2cccc3ccccc23)c2oc3ccccc3c12] | 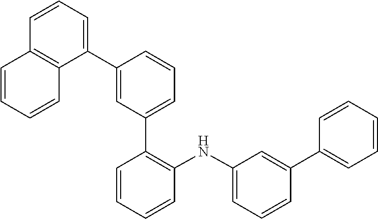 [SMILES: c1ccc(-c2cccc(Nc3ccccc3-c3cccc(-c4cccc5ccccc45)c3)c2)cc1] | |
| P64 | 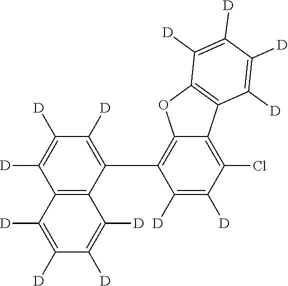 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 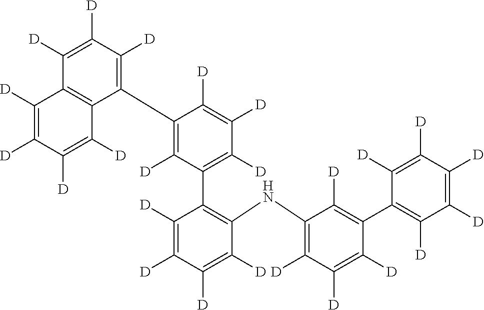 [SMILES: [2H]c1c([2H])c([2H])c(-c2c([2H])c([2H])c([2H])c(Nc3c([2H])c([2H])c([2H])c([2H])c3-c3c([2H])c([2H])c([2H])c(-c4c([2H])c([2H])c([2H])c5c([2H])c([2H])c([2H])c([2H])c45)c3[2H])c2[2H])c([2H])c1[2H]] | |
| P65 | 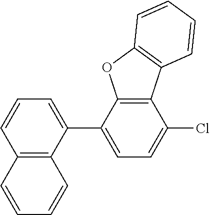 [SMILES: Clc1ccc(-c2cccc3ccccc23)c2oc3ccccc3c12] | 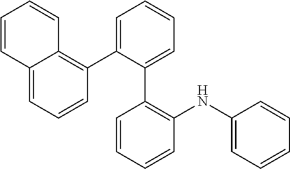 [SMILES: c1ccc(Nc2ccccc2-c2ccccc2-c2cccc3ccccc23)cc1] | |
| P66 | 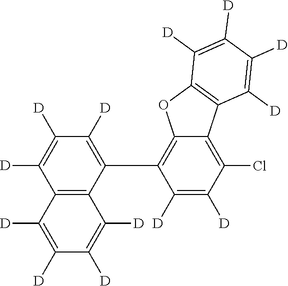 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 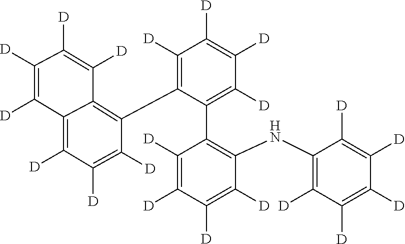 [SMILES: [2H]c1c([2H])c([2H])c(Nc2c([2H])c([2H])c([2H])c([2H])c2-c2c([2H])c([2H])c([2H])c([2H])c2-c2c([2H])c([2H])c([2H])c3c([2H])c([2H])c([2H])c([2H])c23)c([2H])c1[2H]] | |
| P67 | 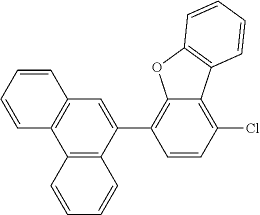 [SMILES: Clc1ccc(-c2cc3ccccc3c3ccccc23)c2oc3ccccc3c12] | 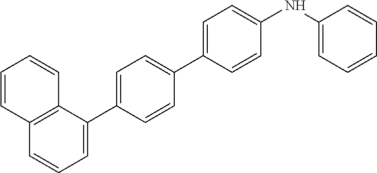 [SMILES: c1ccc(Nc2ccc(-c3ccc(-c4cccc5ccccc45)cc3)cc2)cc1] | |
| P68 | 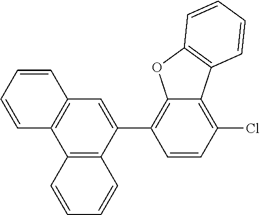 [SMILES: Clc1ccc(-c2cc3ccccc3c3ccccc23)c2oc3ccccc3c12] | 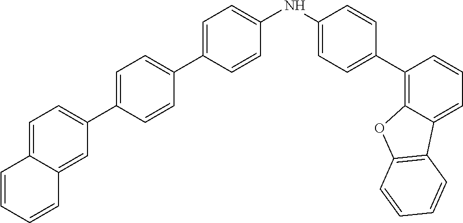 [SMILES: c1ccc2cc(-c3ccc(-c4ccc(Nc5ccc(-c6cccc7c6oc6ccccc67)cc5)cc4)cc3)ccc2c1] | |
| P69 | 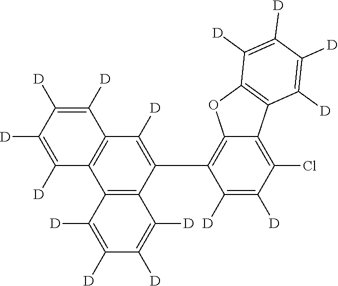 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 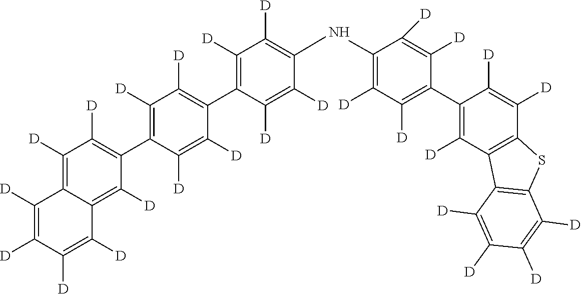 [SMILES: [2H]c1c([2H])c(-c2c([2H])c([2H])c(-c3c([2H])c([2H])c4c([2H])c([2H])c([2H])c([2H])c4c3[2H])c([2H])c2[2H])c([2H])c([2H])c1Nc1c([2H])c([2H])c(-c2c([2H])c([2H])c3sc4c([2H])c([2H])c([2H])c([2H])c4c3c2[2H])c([2H])c1[2H]] | |
| P70 | 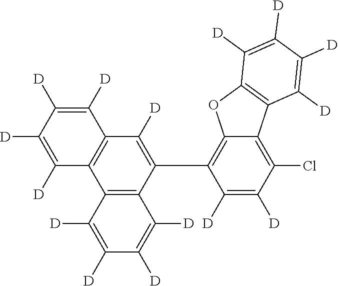 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 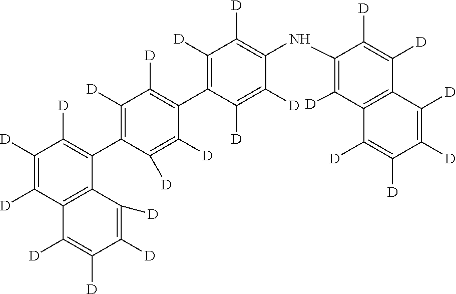 [SMILES: [2H]c1c([2H])c(-c2c([2H])c([2H])c(-c3c([2H])c([2H])c([2H])c4c([2H])c([2H])c([2H])c([2H])c34)c([2H])c2[2H])c([2H])c([2H])c1Nc1c([2H])c([2H])c2c([2H])c([2H])c([2H])c([2H])c2c1[2H]] | |
| P71 | 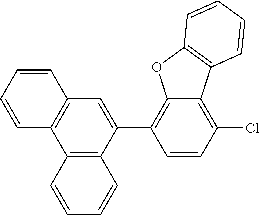 [SMILES: Clc1ccc(-c2cc3ccccc3c3ccccc23)c2oc3ccccc3c12] | 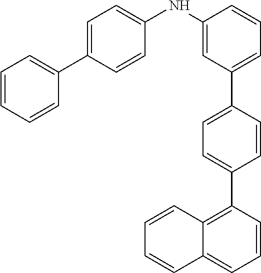 [SMILES: c1ccc(-c2ccc(Nc3cccc(-c4ccc(-c5cccc6ccccc56)cc4)c3)cc2)cc1] | |
| P72 | 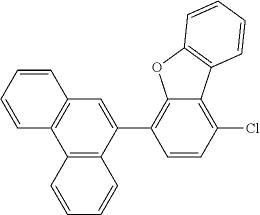 [SMILES: Clc1ccc(-c2cc3ccccc3c3ccccc23)c2oc3ccccc3c12] | 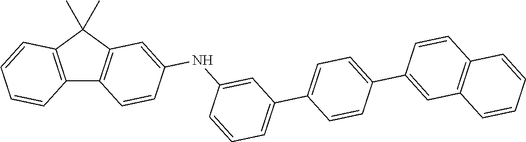 [SMILES: CC1(C)c2ccccc2-c2ccc(Nc3cccc(-c4ccc(-c5ccc6ccccc6c5)cc4)c3)cc21] | |
| P73 | 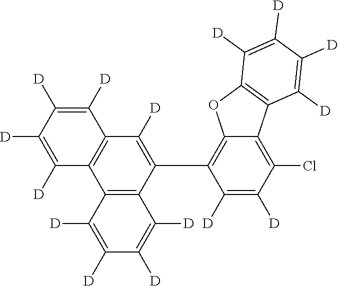 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 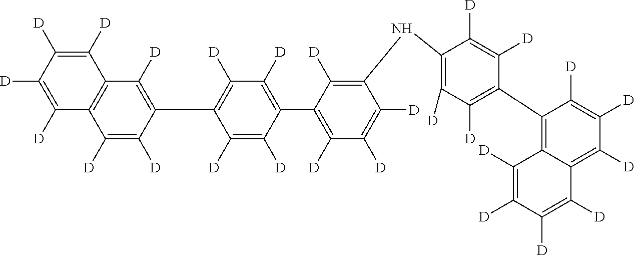 [SMILES: [2H]c1c([2H])c(Nc2c([2H])c([2H])c(-c3c([2H])c([2H])c([2H])c4c([2H])c([2H])c([2H])c([2H])c34)c([2H])c2[2H])c([2H])c(-c2c([2H])c([2H])c(-c3c([2H])c([2H])c4c([2H])c([2H])c([2H])c([2H])c4c3[2H])c([2H])c2[2H])c1[2H]] | |
| P74 | 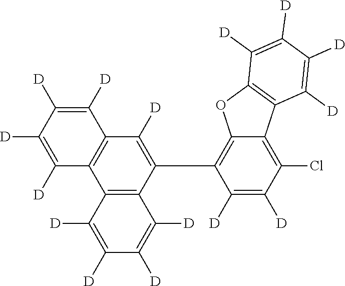 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 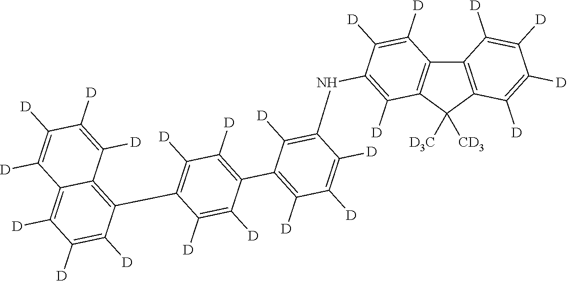 [SMILES: [2H]c1c([2H])c(Nc2c([2H])c([2H])c3c(c2[2H])C(C([2H])([2H])[2H])(C([2H])([2H])[2H])c2c([2H])c([2H])c([2H])c([2H])c2-3)c([2H])c(-c2c([2H])c([2H])c(-c3c([2H])c([2H])c([2H])c4c([2H])c([2H])c([2H])c([2H])c34)c([2H])c2[2H])c1[2H]] | |
| P75 | 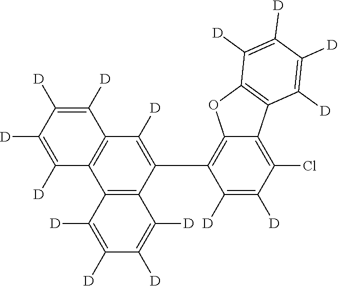 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 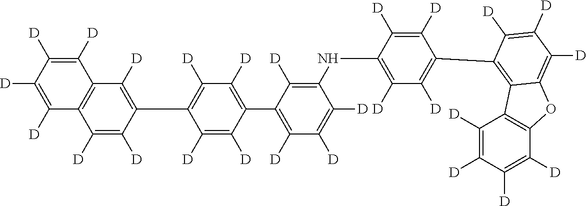 [SMILES: [2H]c1c([2H])c(Nc2c([2H])c([2H])c(-c3c([2H])c([2H])c([2H])c4oc5c([2H])c([2H])c([2H])c([2H])c5c34)c([2H])c2[2H])c([2H])c(-c2c([2H])c([2H])c(-c3c([2H])c([2H])c4c([2H])c([2H])c([2H])c([2H])c4c3[2H])c([2H])c2[2H])c1[2H]] | |
| P76 | 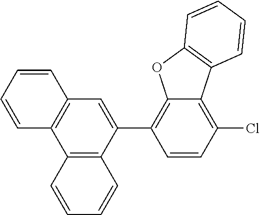 [SMILES: Clc1ccc(-c2cc3ccccc3c3ccccc23)c2oc3ccccc3c12] | 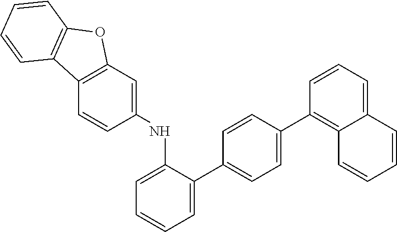 [SMILES: c1ccc(-c2ccc(-c3cccc4ccccc34)cc2)c(Nc2ccc3c(c2)oc2ccccc23)c1] | |
| P77 | 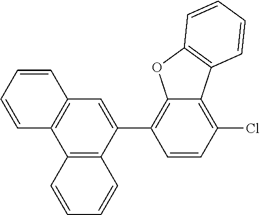 [SMILES: Clc1ccc(-c2cc3ccccc3c3ccccc23)c2oc3ccccc3c12] | 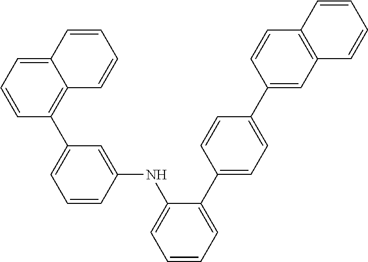 [SMILES: c1cc(Nc2ccccc2-c2ccc(-c3ccc4ccccc4c3)cc2)cc(-c2cccc3ccccc23)c1] | |
| P78 | 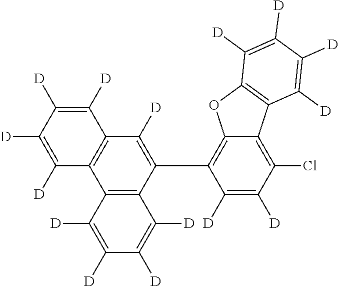 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 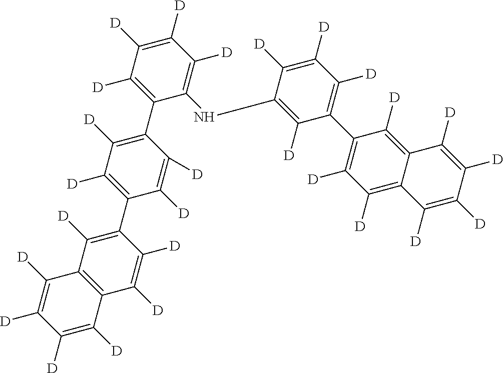 [SMILES: [2H]c1c([2H])c([2H])c(-c2c([2H])c([2H])c(-c3c([2H])c([2H])c4c([2H])c([2H])c([2H])c([2H])c4c3[2H])c([2H])c2[2H])c(Nc2c([2H])c([2H])c([2H])c(-c3c([2H])c([2H])c4c([2H])c([2H])c([2H])c([2H])c4c3[2H])c2[2H])c1[2H]] | |
| P79 | 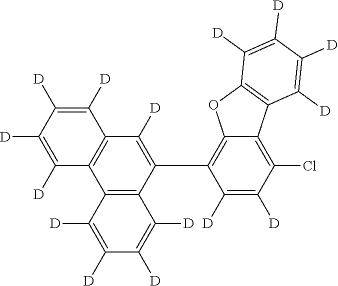 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 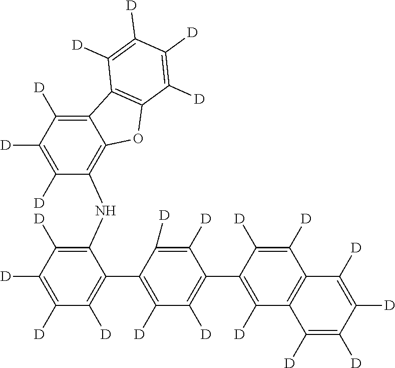 [SMILES: [2H]c1c([2H])c([2H])c(-c2c([2H])c([2H])c(-c3c([2H])c([2H])c4c([2H])c([2H])c([2H])c([2H])c4c3[2H])c([2H])c2[2H])c(Nc2c([2H])c([2H])c([2H])c3c2oc2c([2H])c([2H])c([2H])c([2H])c23)c1[2H]] | |
| P80 | 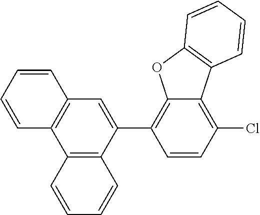 [SMILES: Clc1ccc(-c2cc3ccccc3c3ccccc23)c2oc3ccccc3c12] | 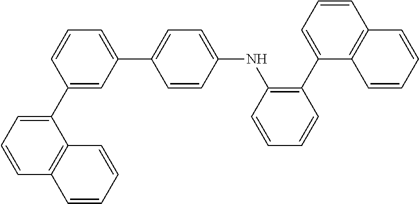 [SMILES: c1cc(-c2ccc(Nc3ccccc3-c3cccc4ccccc34)cc2)cc(-c2cccc3ccccc23)c1] | |
| P81 | 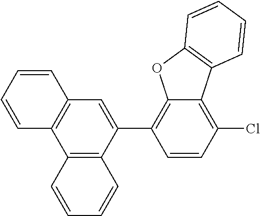 [SMILES: Clc1ccc(-c2cc3ccccc3c3ccccc23)c2oc3ccccc3c12] | 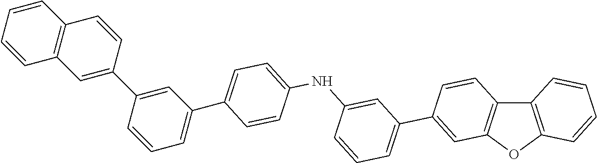 [SMILES: c1cc(Nc2ccc(-c3cccc(-c4ccc5ccccc5c4)c3)cc2)cc(-c2ccc3c(c2)oc2ccccc23)c1] | |
| P82 | 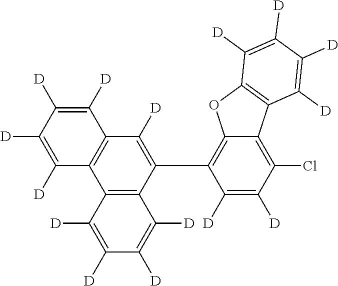 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 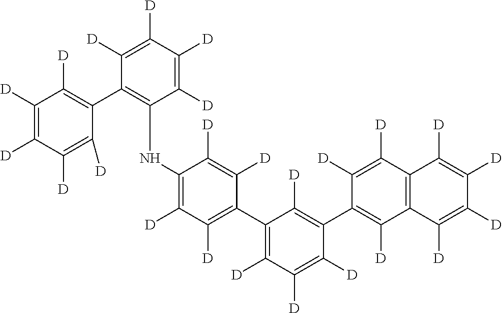 [SMILES: [2H]c1c([2H])c([2H])c(-c2c([2H])c([2H])c([2H])c([2H])c2Nc2c([2H])c([2H])c(-c3c([2H])c([2H])c([2H])c(-c4c([2H])c([2H])c5c([2H])c([2H])c([2H])c([2H])c5c4[2H])c3[2H])c([2H])c2[2H])c([2H])c1[2H]] | |
| P83 | 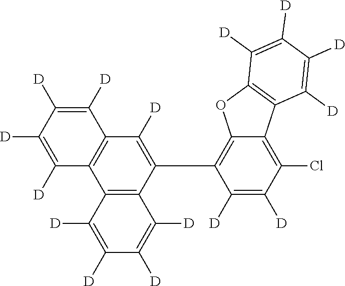 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 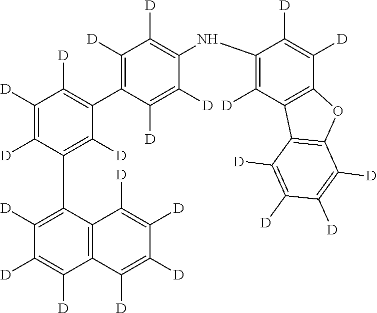 [SMILES: [2H]c1c([2H])c(-c2c([2H])c([2H])c(Nc3c([2H])c([2H])c4oc5c([2H])c([2H])c([2H])c([2H])c5c4c3[2H])c([2H])c2[2H])c([2H])c(-c2c([2H])c([2H])c([2H])c3c([2H])c([2H])c([2H])c([2H])c23)c1[2H]] | |
| P84 | 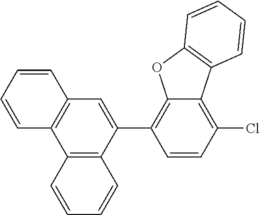 [SMILES: Clc1ccc(-c2cc3ccccc3c3ccccc23)c2oc3ccccc3c12] | 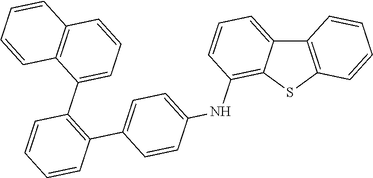 [SMILES: c1ccc(-c2cccc3ccccc23)c(-c2ccc(Nc3cccc4c3sc3ccccc34)cc2)c1] | |
| P85 | 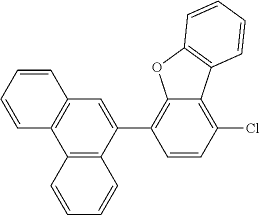 [SMILES: Clc1ccc(-c2cc3ccccc3c3ccccc23)c2oc3ccccc3c12] | 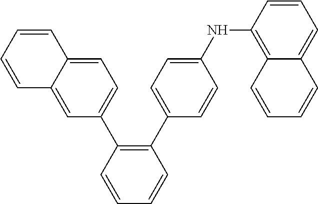 [SMILES: c1ccc(-c2ccc3ccccc3c2)c(-c2ccc(Nc3cccc4ccccc34)cc2)c1] | |
| P86 | 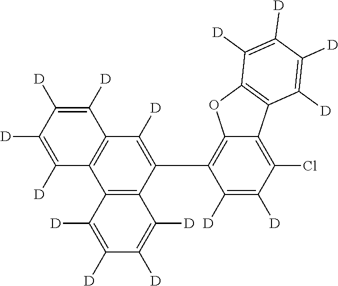 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 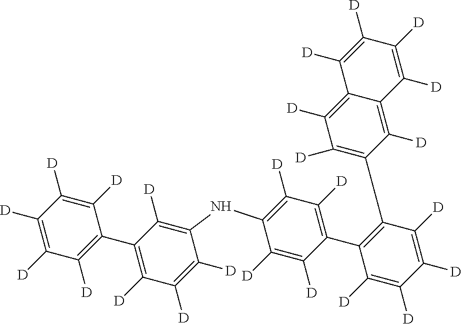 [SMILES: [2H]c1c([2H])c([2H])c(-c2c([2H])c([2H])c([2H])c(Nc3c([2H])c([2H])c(-c4c([2H])c([2H])c([2H])c([2H])c4-c4c([2H])c([2H])c5c([2H])c([2H])c([2H])c([2H])c5c4[2H])c([2H])c3[2H])c2[2H])c([2H])c1[2H]] | |
| P87 | 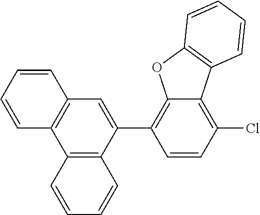 [SMILES: Clc1ccc(-c2cc3ccccc3c3ccccc23)c2oc3ccccc3c12] | 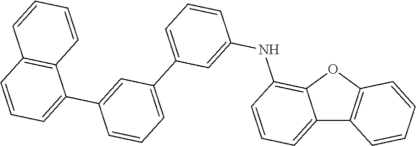 [SMILES: c1cc(Nc2cccc3c2oc2ccccc23)cc(-c2cccc(-c3cccc4ccccc34)c2)c1] | |
| P88 | 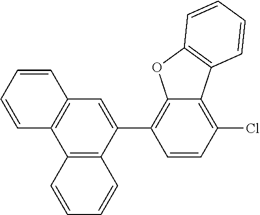 [SMILES: Clc1ccc(-c2cc3ccccc3c3ccccc23)c2oc3ccccc3c12] | 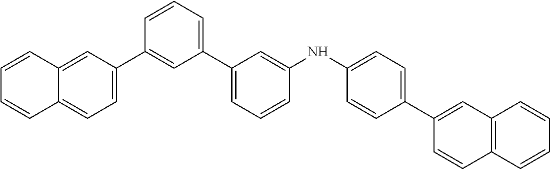 [SMILES: c1cc(Nc2ccc(-c3ccc4ccccc4c3)cc2)cc(-c2cccc(-c3ccc4ccccc4c3)c2)c1] | |
| P89 | 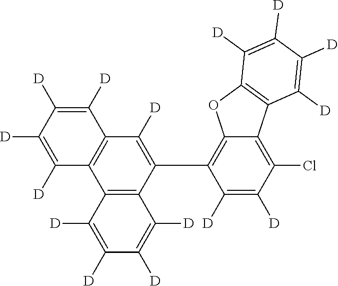 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 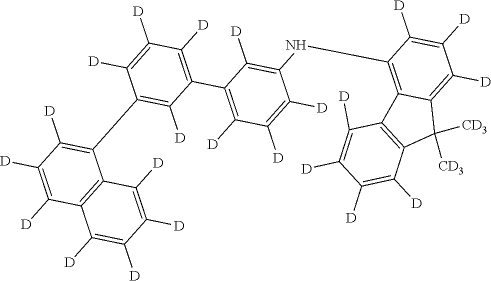 [SMILES: [2H]c1c([2H])c(Nc2c([2H])c([2H])c([2H])c3c2-c2c([2H])c([2H])c([2H])c([2H])c2C3(C([2H])([2H])[2H])C([2H])([2H])[2H])c([2H])c(-c2c([2H])c([2H])c([2H])c(-c3c([2H])c([2H])c([2H])c4c([2H])c([2H])c([2H])c([2H])c34)c2[2H])c1[2H]] | |
| P90 | 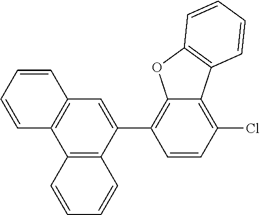 [SMILES: Clc1ccc(-c2cc3ccccc3c3ccccc23)c2oc3ccccc3c12] | 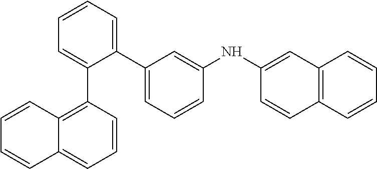 [SMILES: c1cc(Nc2ccc3ccccc3c2)cc(-c2ccccc2-c2cccc3ccccc23)c1] | |
| P91 | 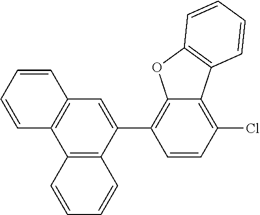 [SMILES: Clc1ccc(-c2cc3ccccc3c3ccccc23)c2oc3ccccc3c12] | 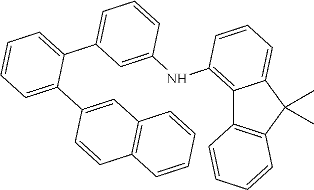 [SMILES: CC1(C)c2ccccc2-c2c(Nc3cccc(-c4ccccc4-c4ccc5ccccc5c4)c3)cccc21] | |
| P92 | 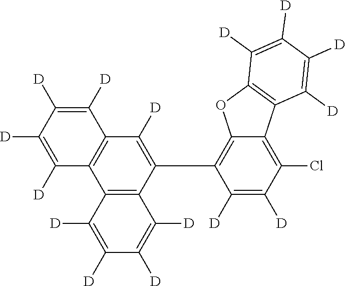 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 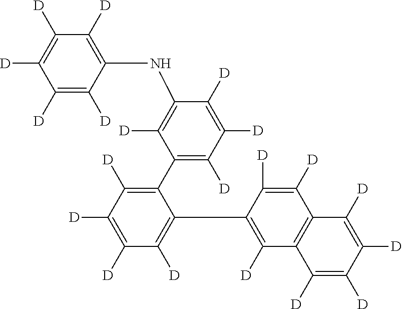 [SMILES: [2H]c1c([2H])c([2H])c(Nc2c([2H])c([2H])c([2H])c(-c3c([2H])c([2H])c([2H])c([2H])c3-c3c([2H])c([2H])c4c([2H])c([2H])c([2H])c([2H])c4c3[2H])c2[2H])c([2H])c1[2H]] | |
| P93 | 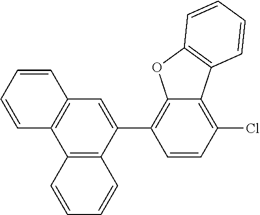 [SMILES: Clc1ccc(-c2cc3ccccc3c3ccccc23)c2oc3ccccc3c12] | 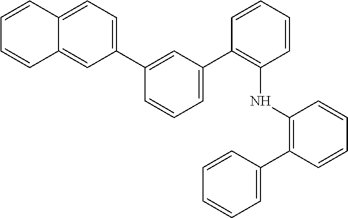 [SMILES: c1ccc(-c2ccccc2Nc2ccccc2-c2cccc(-c3ccc4ccccc4c3)c2)cc1] | |
| P94 | 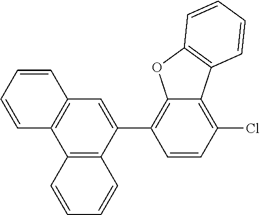 [SMILES: Clc1ccc(-c2cc3ccccc3c3ccccc23)c2oc3ccccc3c12] | 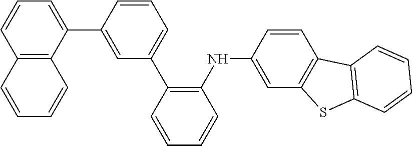 [SMILES: c1cc(-c2ccccc2Nc2ccc3c(c2)sc2ccccc23)cc(-c2cccc3ccccc23)c1] | |
| P95 | 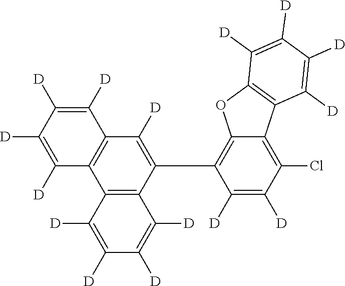 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 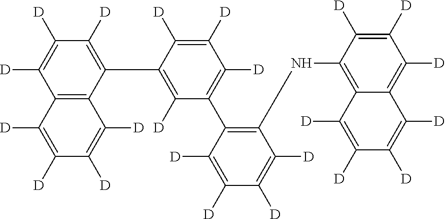 [SMILES: [2H]c1c([2H])c([2H])c(-c2c([2H])c([2H])c([2H])c(-c3c([2H])c([2H])c([2H])c4c([2H])c([2H])c([2H])c([2H])c34)c2[2H])c(Nc2c([2H])c([2H])c([2H])c3c([2H])c([2H])c([2H])c([2H])c23)c1[2H]] | |
| P96 | 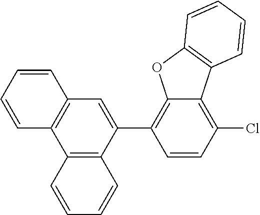 [SMILES: Clc1ccc(-c2cc3ccccc3c3ccccc23)c2oc3ccccc3c12] | 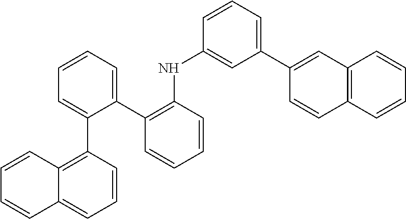 [SMILES: c1cc(Nc2ccccc2-c2ccccc2-c2cccc3ccccc23)cc(-c2ccc3ccccc3c2)c1] | |
| P97 | 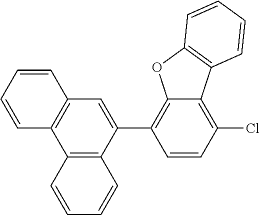 [SMILES: Clc1ccc(-c2cc3ccccc3c3ccccc23)c2oc3ccccc3c12] | 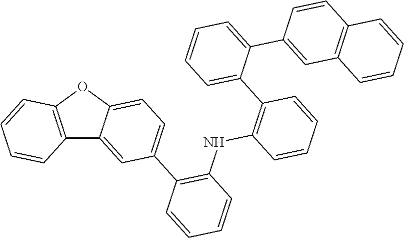 [SMILES: c1ccc(-c2ccc3oc4ccccc4c3c2)c(Nc2ccccc2-c2ccccc2-c2ccc3ccccc3c2)c1] | |
| P98 | 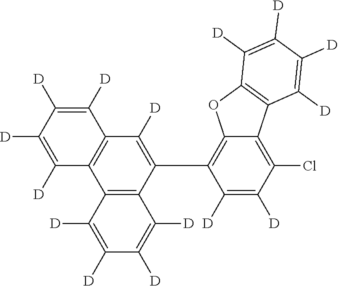 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 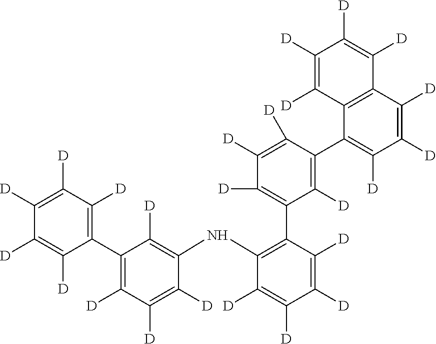 [SMILES: [2H]c1c([2H])c([2H])c(-c2c([2H])c([2H])c([2H])c(Nc3c([2H])c([2H])c([2H])c([2H])c3-c3c([2H])c([2H])c([2H])c(-c4c([2H])c([2H])c([2H])c5c([2H])c([2H])c([2H])c([2H])c45)c3[2H])c2[2H])c([2H])c1[2H]] | |
| P99 | 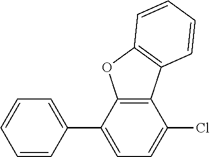 [SMILES: Clc1ccc(-c2ccccc2)c2oc3ccccc3c12] | 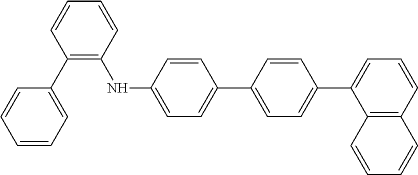 [SMILES: c1ccc(-c2ccccc2Nc2ccc(-c3ccc(-c4cccc5ccccc45)cc3)cc2)cc1] | |
| P100 | 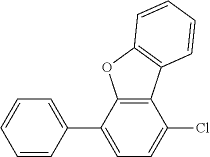 [SMILES: Clc1ccc(-c2ccccc2)c2oc3ccccc3c12] | 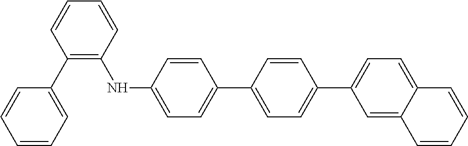 [SMILES: c1ccc(-c2ccccc2Nc2ccc(-c3ccc(-c4ccc5ccccc5c4)cc3)cc2)cc1] | |
| P101 | 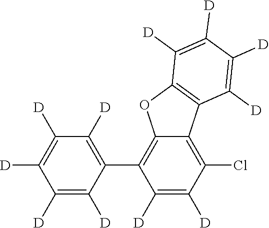 [SMILES: [2H]c1c([2H])c([2H])c(-c2c([2H])c([2H])c(Cl)c3c2oc2c([2H])c([2H])c([2H])c([2H])c23)c([2H])c1[2H]] | 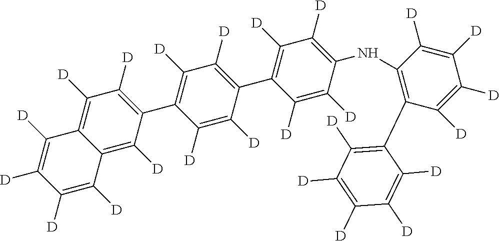 [SMILES: [2H]c1c([2H])c([2H])c(-c2c([2H])c([2H])c([2H])c([2H])c2Nc2c([2H])c([2H])c(-c3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | |
| | | Yield | |
| Item | Products | (%) | [M + H]⁺ |
| P1 | 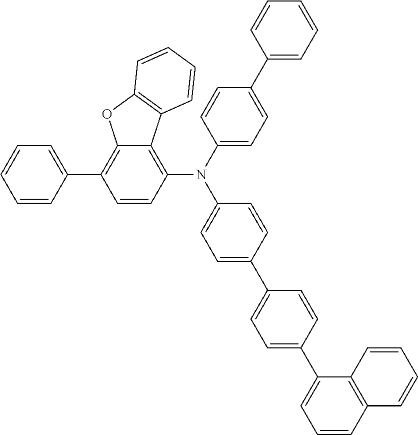 [SMILES: c1ccc(-c2ccc(N(c3ccc(-c4ccc(-c5cccc6ccccc56)cc4)cc3)c3ccc(-c4ccccc4)c4oc5ccccc5c34)cc2)cc1] | 19.0 g (69%) | 689.27 |
| | Compound 1-9 | | |
| P2 | 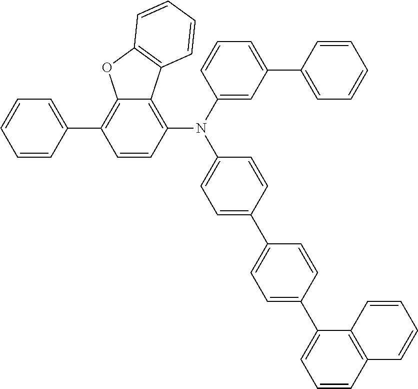 [SMILES: c1ccc(-c2cccc(N(c3ccc(-c4ccc(-c5cccc6ccccc56)cc4)cc3)c3ccc(-c4ccccc4)c4oc5ccccc5c34)c2)cc1] | 19.9 g (72%) | 689.27 |
| | Compound 1-13 | | |
| P3 | 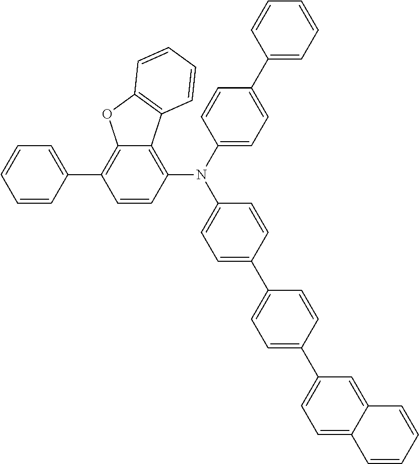 [SMILES: c1ccc(-c2ccc(N(c3ccc(-c4ccc(-c5ccc6ccccc6c5)cc4)cc3)c3ccc(-c4ccccc4)c4oc5ccccc5c34)cc2)cc1] | 20.4 g (74%) | 689.27 |
| | Compound 1-41 | | |
| P4 | 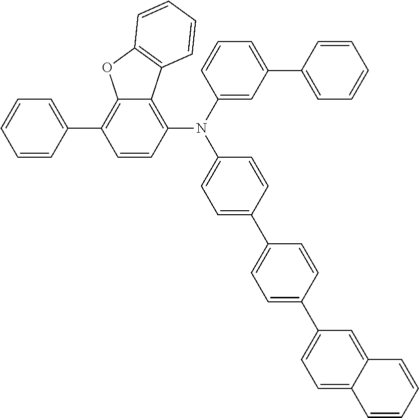 [SMILES: c1ccc(-c2cccc(N(c3ccc(-c4ccc(-c5ccc6ccccc6c5)cc4)cc3)c3ccc(-c4ccccc4)c4oc5ccccc5c34)c2)cc1] | 19.6 g (71%) | 689.27 |
| | Compound 1-45 | | |
| P5 | 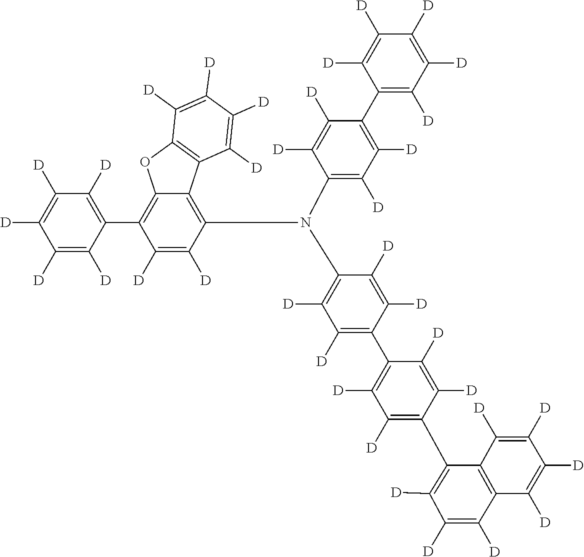 [SMILES: [2H]c1c([2H])c([2H])c(-c2c([2H])c([2H])c(N(c3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c(-c4c([2H])c([2H])c([2H])c([2H])c4[2H])c4oc5c([2H])c([2H])c([2H])c([2H])c5c34)c([2H])c2[2H])c([2H])c1[2H]] | 17.7 g (61%) | 724.49 |
| | Compound 1-67 | | |
| P6 | 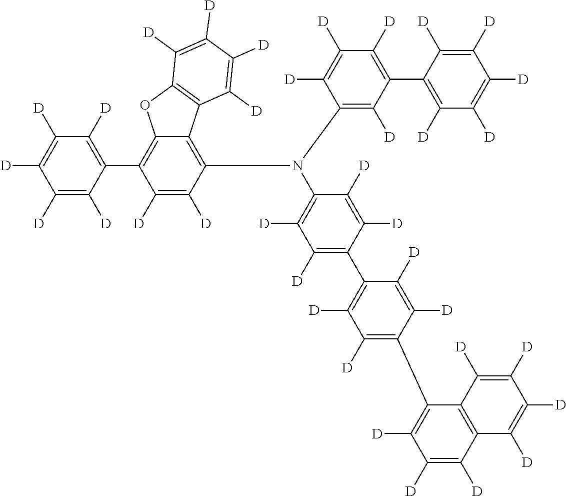 [SMILES: [2H]c1c([2H])c([2H])c(-c2c([2H])c([2H])c([2H])c(N(c3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c(-c4c([2H])c([2H])c([2H])c([2H])c4[2H])c4oc5c([2H])c([2H])c([2H])c([2H])c5c34)c2[2H])c([2H])c1[2H]] | 18.9 g (65%) | 724.49 |
| | Compound 1-70 | | |
| P7 | 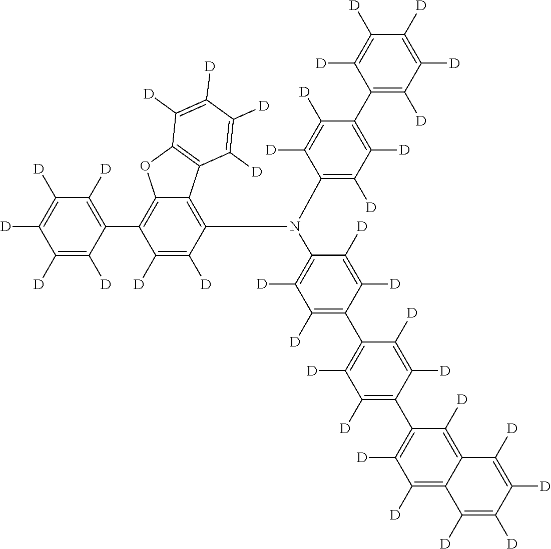 [SMILES: [2H]c1c([2H])c([2H])c(-c2c([2H])c([2H])c(N(c3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c3c([2H])c([2H])c(-c4c([2H])c([2H])c([2H])c([2H])c4[2H])c4oc5c([2H])c([2H])c([2H])c([2H])c5c34)c([2H])c2[2H])c([2H])c1[2H]] | 20.0 g (69%) | 724.49 |
| | Compound 1-73 | | |
| P8 | 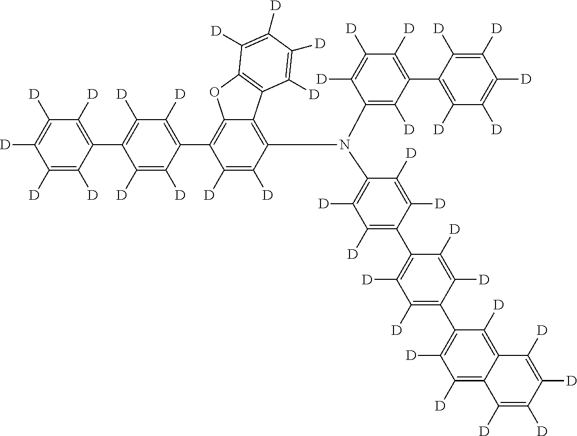 [SMILES: [2H]c1c([2H])c([2H])c(-c2c([2H])c([2H])c(-c3c([2H])c([2H])c(N(c4c([2H])c([2H])c(-c5c([2H])c([2H])c(-c6c([2H])c([2H])c7c([2H])c([2H])c([2H])c([2H])c7c6[2H])c([2H])c5[2H])c([2H])c4[2H])c4c([2H])c([2H])c([2H])c(-c5c([2H])c([2H])c([2H])c([2H])c5[2H])c4[2H])c4c3oc3c([2H])c([2H])c([2H])c([2H])c34)c([2H])c2[2H])c([2H])c1[2H]] | 18.6 g (64%) | 724.49 |
| | Compound 1-76 | | |
| P9 | 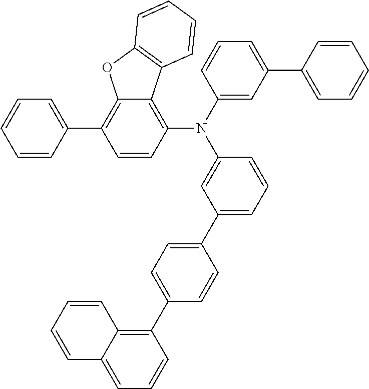 [SMILES: c1ccc(-c2cccc(N(c3cccc(-c4ccc(-c5cccc6ccccc56)cc4)c3)c3ccc(-c4ccccc4)c4oc5ccccc5c34)c2)cc1] | 20.1 g (73%) | 689.27 |
| | Compound 1-92 | | |
| P10 | 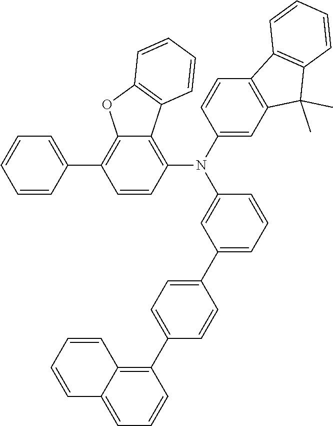 [SMILES: CC1(C)c2ccccc2-c2ccc(N(c3cccc(-c4ccc(-c5cccc6ccccc56)cc4)c3)c3ccc(-c4ccccc4)c4oc5ccccc5c34)cc21] | 20.7 g (71%) | 729.30 |
| | Compound 1-85 | | |
| P11 | 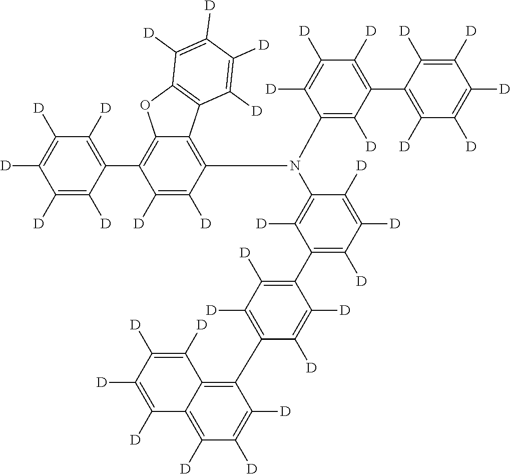 [SMILES: [2H]c1c([2H])c([2H])c(-c2c([2H])c([2H])c([2H])c(N(c3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])c3c([2H])c([2H])c(-c4c([2H])c([2H])c([2H])c([2H])c4[2H])c4oc5c([2H])c([2H])c([2H])c([2H])c5c34)c2[2H])c([2H])c1[2H]] | 18.3 g (63%) | 724.49 |
| | Compound 1-149 | | |
| P12 | 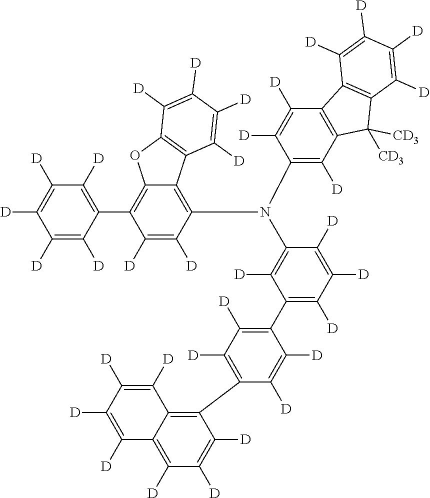 [SMILES: [2H]c1c([2H])c([2H])c(-c2c([2H])c([2H])c(N(c3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])c3c([2H])c([2H])c4c(c3[2H])C(C([2H])([2H])[2H])(C([2H])([2H])[2H])c3c([2H])c([2H])c([2H])c([2H])c3-4)c3c2oc2c([2H])c([2H])c([2H])c([2H])c23)c([2H])c1[2H]] | 19.1 g (62%) | 768.55 |
| | Compound 1-152 | | |
| P13 | 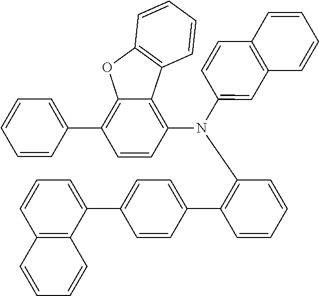 [SMILES: c1ccc(-c2ccc(N(c3ccc4ccccc4c3)c3ccccc3-c3ccc(-c4cccc5ccccc45)cc3)c3c2oc2ccccc23)cc1] | 18.6 g (70%) | 663.26 |
| | Compound 1-155 | | |
| P14 | 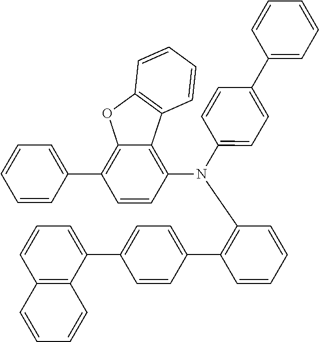 [SMILES: c1ccc(-c2ccc(N(c3ccccc3-c3ccc(-c4cccc5ccccc45)cc3)c3ccc(-c4ccccc4)c4oc5ccccc5c34)cc2)cc1] | 19.9 g (72%) | 689.27 |
| | Compound 1-161 | | |
| P15 | 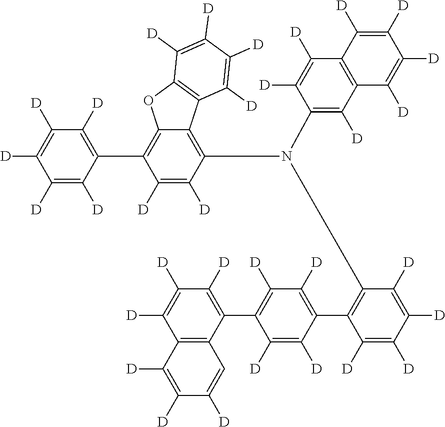 [SMILES: [2H]c1cc2c(-c3c([2H])c([2H])c(-c4c([2H])c([2H])c([2H])c([2H])c4N(c4c([2H])c([2H])c5c([2H])c([2H])c([2H])c([2H])c5c4[2H])c4c([2H])c([2H])c(-c5c([2H])c([2H])c([2H])c([2H])c5[2H])c5oc6c([2H])c([2H])c([2H])c([2H])c6c45)c([2H])c3[2H])c([2H])c([2H])c([2H])c2c([2H])c1[2H]] | 16.7 g (60%) | 695.46 |
| | Compound 1-219 | | |
| P16 | 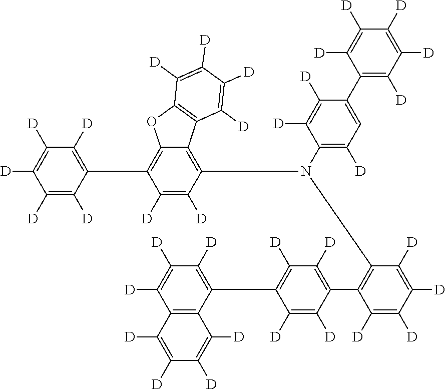 [SMILES: [2H]c1cc(-c2c([2H])c([2H])c([2H])c([2H])c2[2H])c([2H])c([2H])c1N(c1c([2H])c([2H])c([2H])c([2H])c1-c1c([2H])c([2H])c(-c2c([2H])c([2H])c([2H])c3c([2H])c([2H])c([2H])c([2H])c23)c([2H])c1[2H])c1c([2H])c([2H])c(-c2c([2H])c([2H])c([2H])c([2H])c2[2H])c2oc3c([2H])c([2H])c([2H])c([2H])c3c12] | 18.6 g (64%) | 724.49 |
| | Compound 1-222 | | |
| P17 | 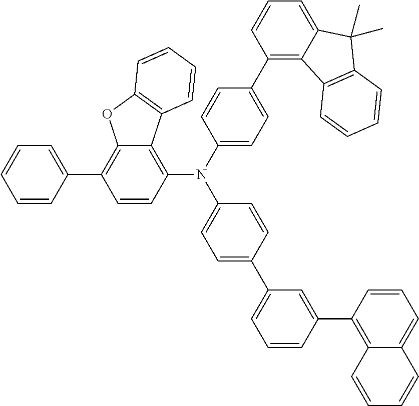 [SMILES: CC1(C)c2ccccc2-c2c(-c3ccc(N(c4ccc(-c5cccc(-c6cccc7ccccc67)c5)cc4)c4ccc(-c5ccccc5)c5oc6ccccc6c45)cc3)cccc21] | 23.2 g (72%) | 805.33 |
| | Compound 1-249 | | |
| P18 | 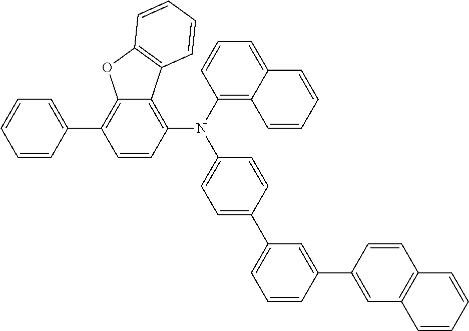 [SMILES: c1ccc(-c2ccc(N(c3ccc(-c4cccc(-c5ccc6ccccc6c5)c4)cc3)c3cccc4ccccc34)c3c2oc2ccccc23)cc1] | 18.9 g (71%) | 663.26 |
| | Compound 1-257 | | |
| P19 | 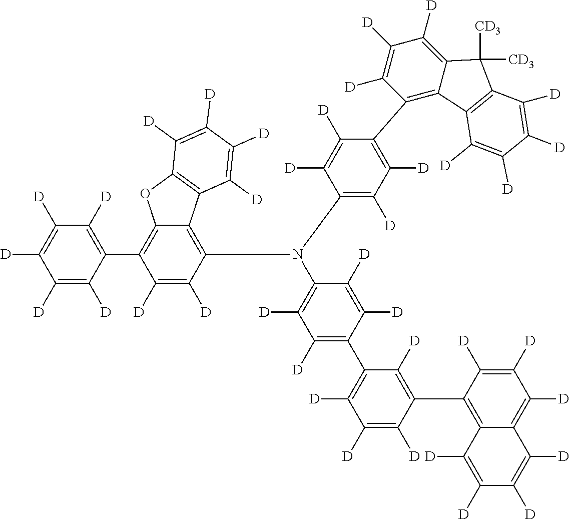 [SMILES: [2H]c1c([2H])c([2H])c(-c2c([2H])c([2H])c(N(c3c([2H])c([2H])c(-c4c([2H])c([2H])c([2H])c(-c5c([2H])c([2H])c([2H])c6c([2H])c([2H])c([2H])c([2H])c56)c4[2H])c([2H])c3[2H])c3c([2H])c([2H])c(-c4c([2H])c([2H])c([2H])c5c4-c4c([2H])c([2H])c([2H])c([2H])c4C5(C([2H])([2H])[2H])C([2H])([2H])[2H])c([2H])c3[2H])c3c2oc2c([2H])c([2H])c([2H])c([2H])c23)c([2H])c1[2H]] | 22.8 g (67%) | 846.60 |
| | Compound 1-288 | | |
| P20 | 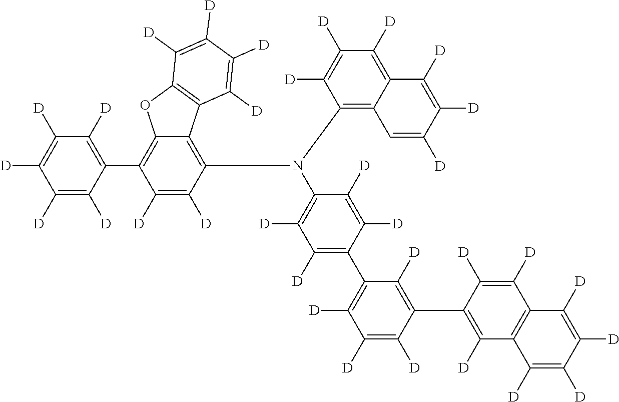 [SMILES: [2H]c1cc2c(N(c3c([2H])c([2H])c(-c4c([2H])c([2H])c([2H])c(-c5c([2H])c([2H])c6c([2H])c([2H])c([2H])c([2H])c6c5[2H])c4[2H])c([2H])c3[2H])c3c([2H])c([2H])c(-c4c([2H])c([2H])c([2H])c([2H])c4[2H])c4oc5c([2H])c([2H])c([2H])c([2H])c5c34)c([2H])c([2H])c([2H])c2c([2H])c1[2H]] | 18.1 g (65%) | 696.46 |
| | Compound 1-291 | | |
| P21 | 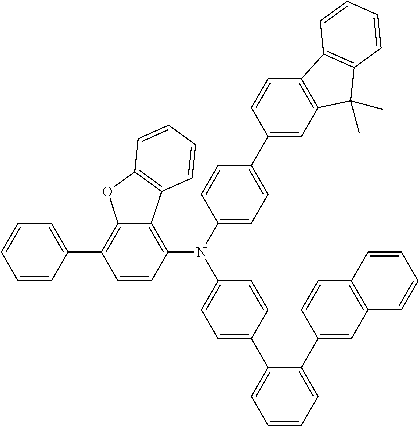 [SMILES: CC1(C)c2ccccc2-c2ccc(-c3ccc(N(c4ccc(-c5ccccc5-c5ccc6ccccc6c5)cc4)c4ccc(-c5ccccc5)c5oc6ccccc6c45)cc3)cc21] | 22.2 g (69%) | 805.33 |
| | Compound 1-338 | | |
| P22 | 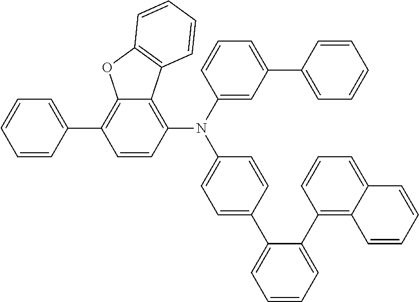 [SMILES: c1ccc(-c2cccc(N(c3ccc(-c4ccccc4-c4cccc5ccccc45)cc3)c3ccc(-c4ccccc4)c4oc5ccccc5c34)c2)cc1] | 18.8 g (68%) | 689.27 |
| | Compound 1-303 | | |
| P23 | 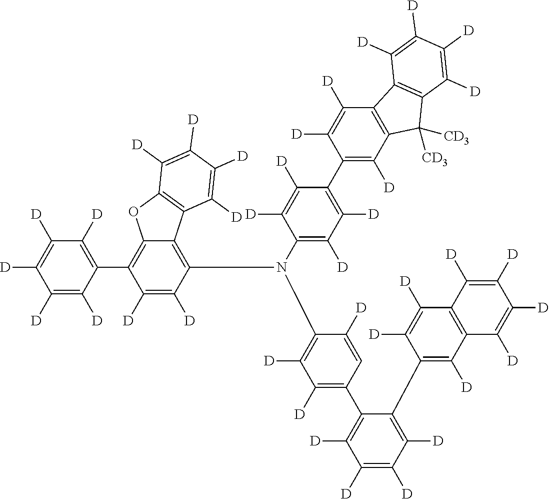 [SMILES: [2H]c1cc(-c2c([2H])c([2H])c([2H])c([2H])c2-c2c([2H])c([2H])c3c([2H])c([2H])c([2H])c([2H])c3c2[2H])c([2H])c([2H])c1N(c1c([2H])c([2H])c(-c2c([2H])c([2H])c3c(c2[2H])C(C([2H])([2H])[2H])(C([2H])([2H])[2H])c2c([2H])c([2H])c([2H])c([2H])c2-3)c([2H])c1[2H])c1c([2H])c([2H])c(-c2c([2H])c([2H])c([2H])c([2H])c2[2H])c2oc3c([2H])c([2H])c([2H])c([2H])c3c12] | 21.7 g (64%) | 847.60 |
| | Compound 1-502 | | |
| P24 | 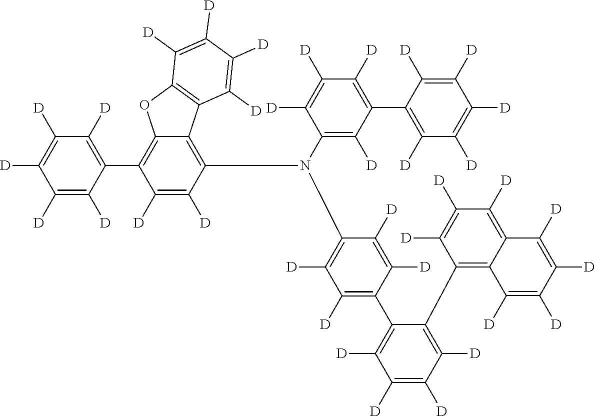 [SMILES: [2H]c1c([2H])c([2H])c(-c2c([2H])c([2H])c([2H])c(N(c3c([2H])c([2H])c(-c4c([2H])c([2H])c([2H])c([2H])c4-c4c([2H])c([2H])c([2H])c5c([2H])c([2H])c([2H])c([2H])c45)c([2H])c3[2H])c3c([2H])c([2H])c(-c4c([2H])c([2H])c([2H])c([2H])c4[2H])c4oc5c([2H])c([2H])c([2H])c([2H])c5c34)c2[2H])c([2H])c1[2H]] | 18.3 g (63%) | 724.49 |
| | Compound 1-349 | | |
| P25 | 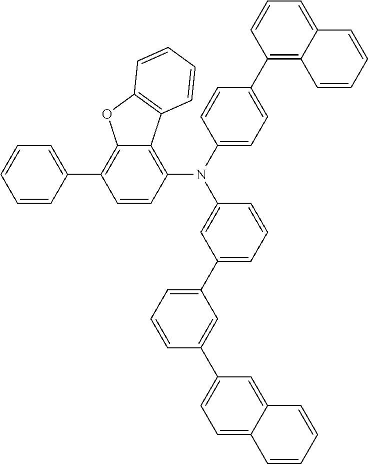 [SMILES: c1ccc(-c2ccc(N(c3ccc(-c4cccc5ccccc45)cc3)c3cccc(-c4cccc(-c5ccc6ccccc6c5)c4)c3)c3c2oc2ccccc23)cc1] | 20.7 g (70%) | 739.29 |
| | Compound 1-376 | | |
| P26 | 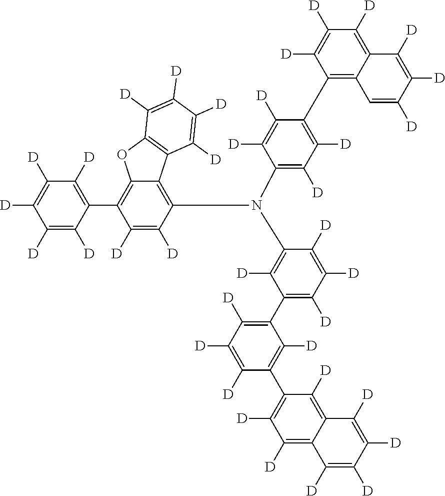 [SMILES: [2H]c1cc2c(-c3c([2H])c([2H])c(N(c4c([2H])c([2H])c([2H])c(-c5c([2H])c([2H])c([2H])c(-c6c([2H])c([2H])c7c([2H])c([2H])c([2H])c([2H])c7c6[2H])c5[2H])c4[2H])c4c([2H])c([2H])c(-c5c([2H])c([2H])c([2H])c([2H])c5[2H])c5oc6c([2H])c([2H])c([2H])c([2H])c6c45)c([2H])c3[2H])c([2H])c([2H])c([2H])c2c([2H])c1[2H]] | 22.4 g (72%) | 776.52 |
| | Compound 1-392 | | |
| P27 | 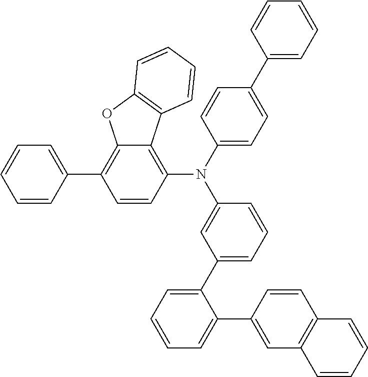 | 17.7 g (64%) | 689.27 |
| | Compound 1-418 | | |
| P28 | 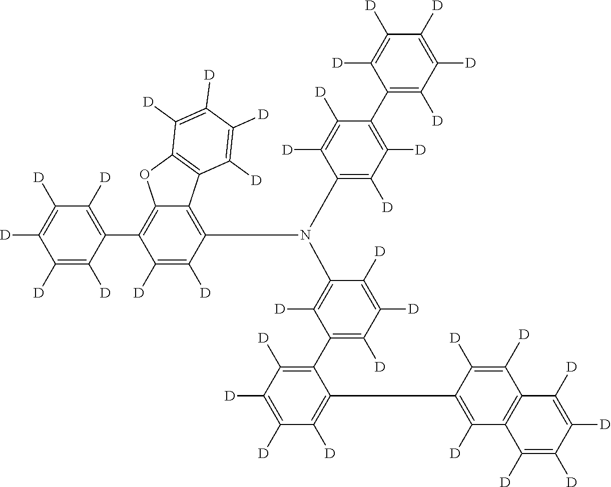 [SMILES: [2H]c1c([2H])c([2H])c(-c2c([2H])c([2H])c(N(c3c([2H])c([2H])c([2H])c(-c4c([2H])c([2H])c([2H])c([2H])c4-c4c([2H])c([2H])c5c([2H])c([2H])c([2H])c([2H])c5c4[2H])c3[2H])c3c([2H])c([2H])c(-c4c([2H])c([2H])c([2H])c([2H])c4[2H])c4oc5c([2H])c([2H])c([2H])c([2H])c5c34)c([2H])c2[2H])c([2H])c1[2H]] | 19.7 g (68%) | 724.49 |
| | Compound 1-435 | | |
| P29 | 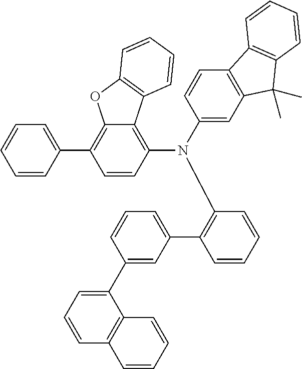 [SMILES: CC1(C)c2ccccc2-c2ccc(N(c3ccccc3-c3cccc(-c4cccc5ccccc45)c3)c3ccc(-c4ccccc4)c4oc5ccccc5c34)cc21] | 19.0 g (65%) | 729.30 |
| | Compound 1-440 | | |
| P30 | 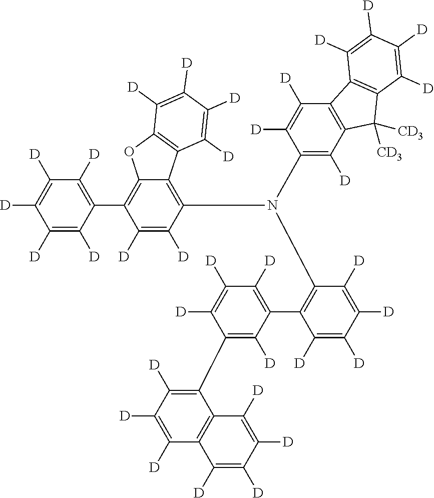 [SMILES: [2H]c1c([2H])c([2H])c(-c2c([2H])c([2H])c(N(c3c([2H])c([2H])c4c(c3[2H])C(C([2H])([2H])[2H])(C([2H])([2H])[2H])c3c([2H])c([2H])c([2H])c([2H])c3-4)c3c([2H])c([2H])c([2H])c([2H])c3-c3c([2H])c([2H])c([2H])c(-c4c([2H])c([2H])c([2H])c5c([2H])c([2H])c([2H])c([2H])c45)c3[2H])c3c2oc2c([2H])c([2H])c([2H])c([2H])c23)c([2H])c1[2H]] | 20.3 g (66%) | 768.55 |
| | Compound 1-469 | | |
| P31 | 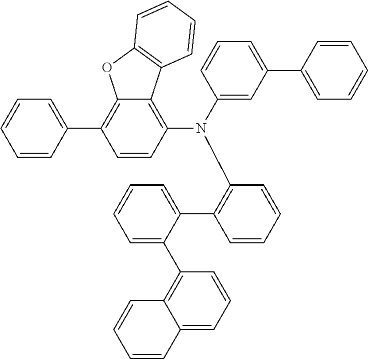 [SMILES: c1ccc(-c2cccc(N(c3ccccc3-c3ccccc3-c3cccc4ccccc34)c3ccc(-c4ccccc4)c4oc5ccccc5c34)c2)cc1] | 17.4 g (63%) | 689.27 |
| | Compound 1-480 | | |
| P32 | 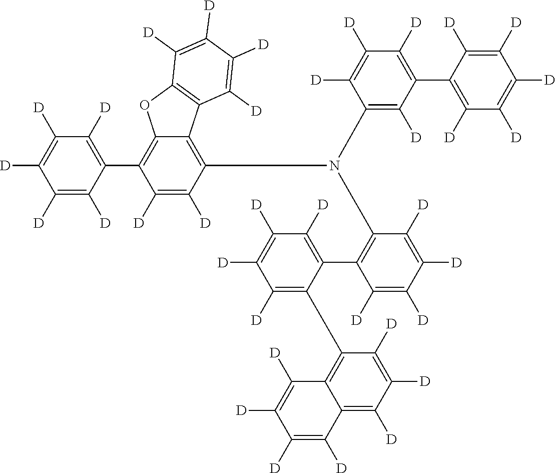 [SMILES: [2H]c1c([2H])c([2H])c(-c2c([2H])c([2H])c([2H])c(N(c3c([2H])c([2H])c([2H])c([2H])c3-c3c([2H])c([2H])c([2H])c([2H])c3-c3c([2H])c([2H])c([2H])c4c([2H])c([2H])c([2H])c([2H])c34)c3c([2H])c([2H])c(-c4c([2H])c([2H])c([2H])c([2H])c4[2H])c4oc5c([2H])c([2H])c([2H])c([2H])c5c34)c2[2H])c([2H])c1[2H]] | 18.0 g (62%) | 724.49 |
| | Compound 1-501 | | |
| P33 | 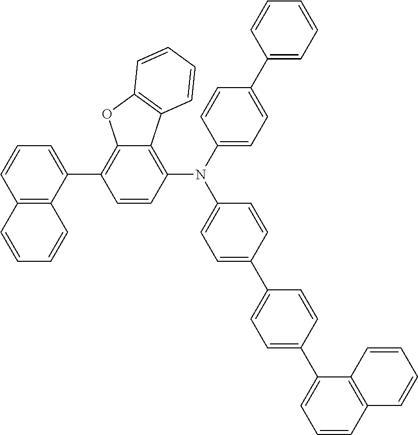 [SMILES: c1ccc(-c2ccc(N(c3ccc(-c4ccc(-c5cccc6ccccc56)cc4)cc3)c3ccc(-c4cccc5ccccc45)c4oc5ccccc5c34)cc2)cc1] | 21.6 g (73%) | 739.29 |
| | Compound 2-9 | | |
| P34 | 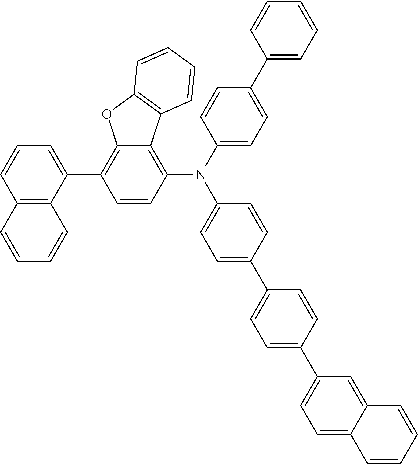 [SMILES: c1ccc(-c2ccc(N(c3ccc(-c4ccc(-c5ccc6ccccc6c5)cc4)cc3)c3ccc(-c4cccc5ccccc45)c4oc5ccccc5c34)cc2)cc1] | 20.7 g (70%) | 739.29 |
| | Compound 2-41 | | |
| P35 | 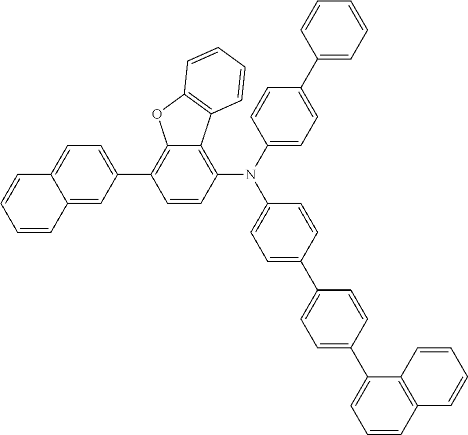 [SMILES: c1ccc(-c2ccc(N(c3ccc(-c4ccc(-c5cccc6ccccc56)cc4)cc3)c3ccc(-c4ccc5ccccc5c4)c4oc5ccccc5c34)cc2)cc1] | 19.8 g (67%) | 739.29 |
| | Compound 2-73 | | |
| P36 | 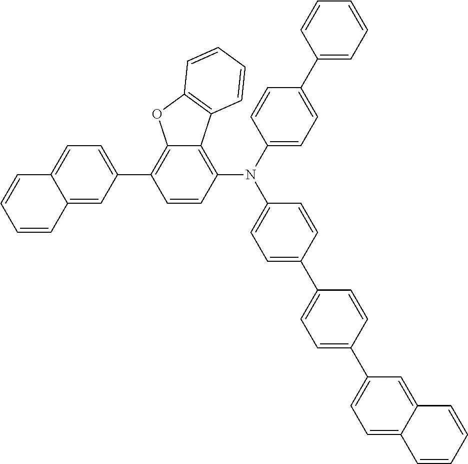 [SMILES: c1ccc(-c2ccc(N(c3ccc(-c4ccc(-c5ccc6ccccc6c5)cc4)cc3)c3ccc(-c4ccc5ccccc5c4)c4oc5ccccc5c34)cc2)cc1] | 21.0 g (71%) | 739.29 |
| | Compound 2-93 | | |
| P37 | 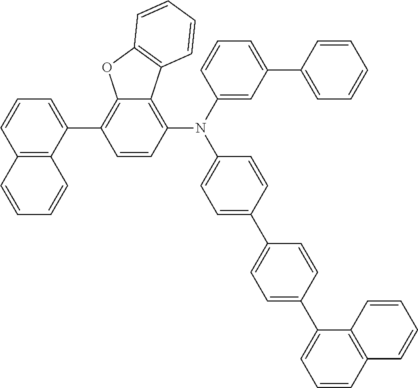 [SMILES: c1ccc(-c2cccc(N(c3ccc(-c4ccc(-c5cccc6ccccc56)cc4)cc3)c3ccc(-c4cccc5ccccc45)c4oc5ccccc5c34)c2)cc1] | 21.3 g (72%) | 739.29 |
| | Compound 2-13 | | |
| P38 | 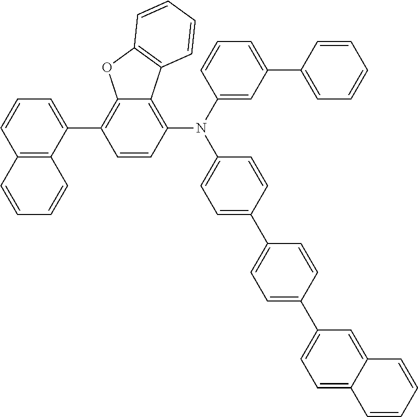 [SMILES: c1ccc(-c2cccc(N(c3ccc(-c4ccc(-c5ccc6ccccc6c5)cc4)cc3)c3ccc(-c4cccc5ccccc45)c4oc5ccccc5c34)c2)cc1] | 21.9 g (74%) | 739.29 |
| | Compound 2-45 | | |
| P39 | 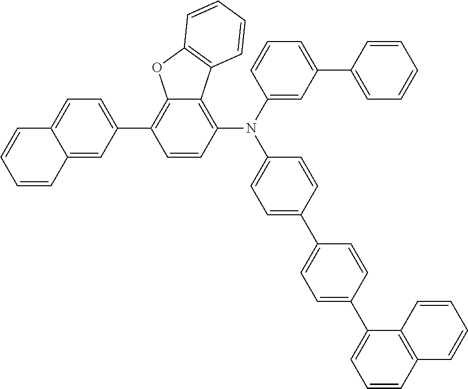 [SMILES: c1ccc(-c2cccc(N(c3ccc(-c4ccc(-c5cccc6ccccc56)cc4)cc3)c3ccc(-c4ccc5ccccc5c4)c4oc5ccccc5c34)c2)cc1] | 20.4 g (69%) | 739.29 |
| | Compound 2-77 | | |
| P40 | 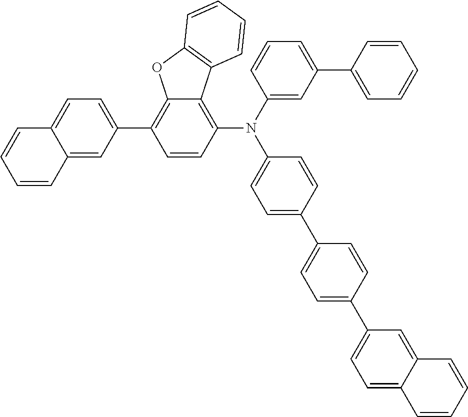 [SMILES: c1ccc(-c2cccc(N(c3ccc(-c4ccc(-c5ccc6ccccc6c5)cc4)cc3)c3ccc(-c4ccc5ccccc5c4)c4oc5ccccc5c34)c2)cc1] | 19.8 g (67%) | 739.29 |
| | Compound 2-97 | | |
| P41 | 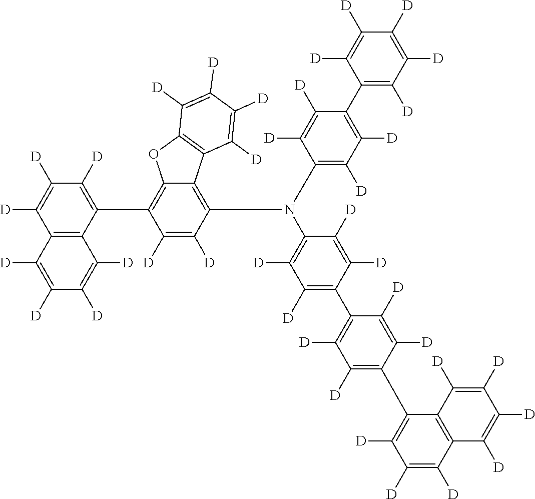 [SMILES: [2H]c1c([2H])c([2H])c(-c2c([2H])c([2H])c(N(c3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H]] | 21.4 g (69%) | 776.52 |
| | Compound 2-107 | | |
| P42 | 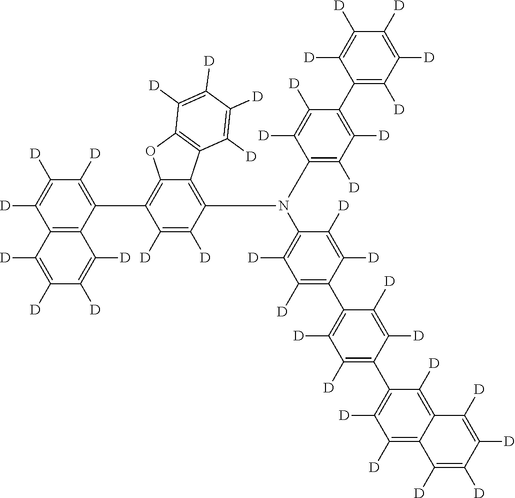 [SMILES: [2H]c1c([2H])c([2H])c(-c2c([2H])c([2H])c(N(c3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H]] | 20.5 g (66%) | 776.52 |
| | Compound 2-110 | | |
| P43 | 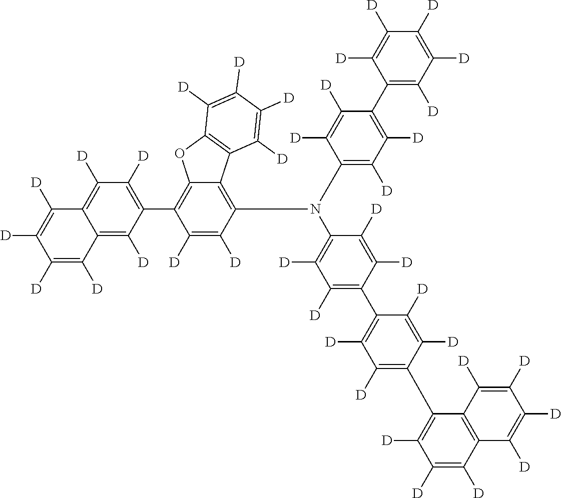 [SMILES: [2H]c1c([2H])c([2H])c(-c2c([2H])c([2H])c(N(c3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c(-c4c([2H])c([2H])c5c([2H])c([2H])c([2H])c([2H])c5c4[2H])c4oc5c([2H])c([2H])c([2H])c([2H])c5c34)c([2H])c2[2H])c([2H])c1[2H]] | 19.9 g (64%) | 776.52 |
| | Compound 2-113 | | |
| P44 | 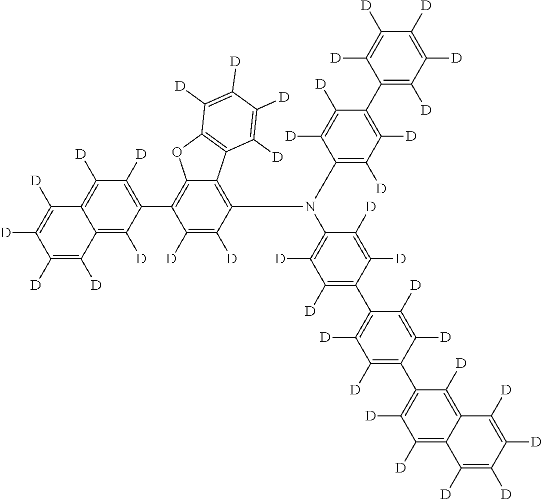 [SMILES: [2H]c1c([2H])c([2H])c(-c2c([2H])c([2H])c(N(c3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c3c([2H])c([2H])c(-c4c([2H])c([2H])c5c([2H])c([2H])c([2H])c([2H])c5c4[2H])c4oc5c([2H])c([2H])c([2H])c([2H])c5c34)c([2H])c2[2H])c([2H])c1[2H]] | 21.8 g (70%) | 776.52 |
| | Compound 2-116 | | |
| P45 | 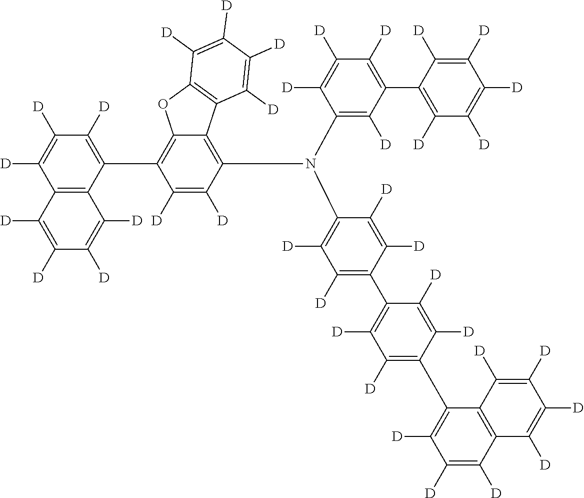 [SMILES: [2H]c1c([2H])c([2H])c(-c2c([2H])c([2H])c([2H])c(N(c3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c2[2H])c([2H])c1[2H]] | 21.4 g (69%) | 776.52 |
| | Compound 2-119 | | |
| P46 | 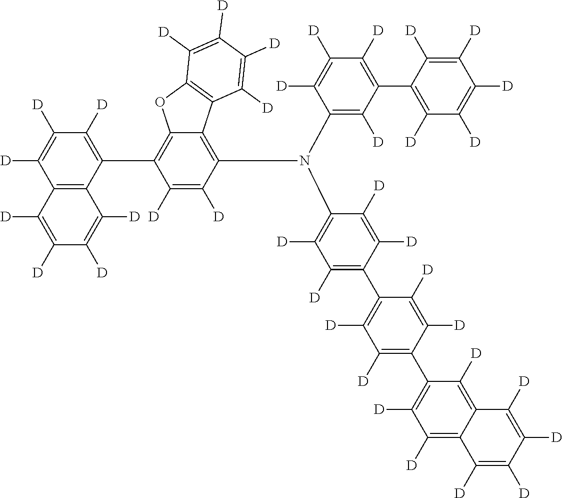 [SMILES: [2H]c1c([2H])c([2H])c(-c2c([2H])c([2H])c([2H])c(N(c3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c2[2H])c([2H])c1[2H]] | 20.5 g (66%) | 776.52 |
| | Compound 2-122 | | |
| P47 | 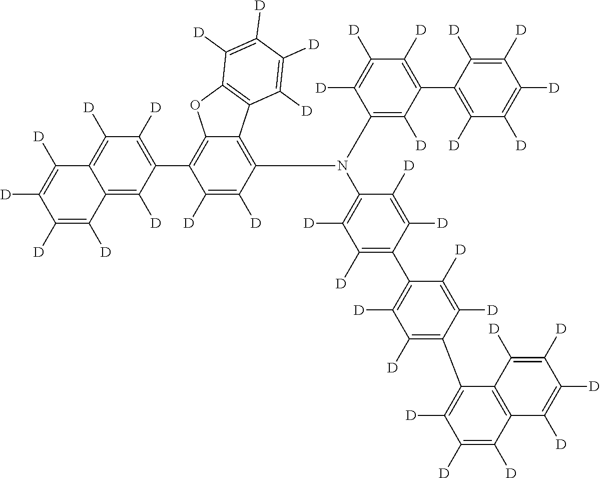 [SMILES: [2H]c1c([2H])c([2H])c(-c2c([2H])c([2H])c([2H])c(N(c3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c(-c4c([2H])c([2H])c5c([2H])c([2H])c([2H])c([2H])c5c4[2H])c4oc5c([2H])c([2H])c([2H])c([2H])c5c34)c2[2H])c([2H])c1[2H]] | 20.2 g (65%) | 776.52 |
| | Compound 2-125 | | |
| P48 | 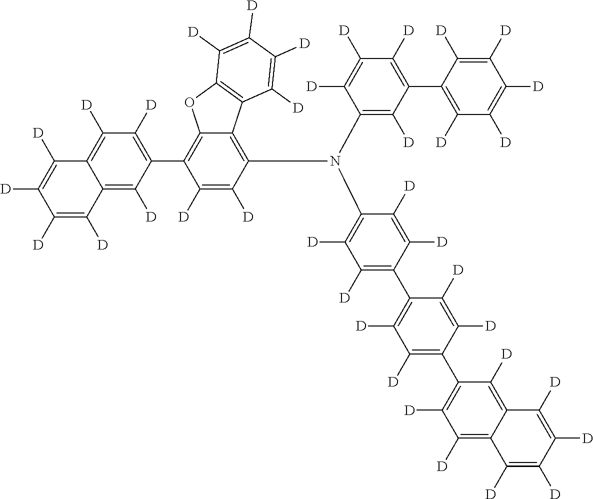 [SMILES: [2H]c1c([2H])c([2H])c(-c2c([2H])c([2H])c([2H])c(N(c3c([2H])c([2H])c(-c4c([2H])c([2H])c(-c5c([2H])c([2H])c6c([2H])c([2H])c([2H])c([2H])c6c5[2H])c([2H])c4[2H])c([2H])c3[2H])c3c([2H])c([2H])c(-c4c([2H])c([2H])c5c([2H])c([2H])c([2H])c([2H])c5c4[2H])c4oc5c([2H])c([2H])c([2H])c([2H])c5c34)c2[2H])c([2H])c1[2H]] | 21.4 g (69%) | 776.52 |
| | Compound 2-128 | | |
| P49 | 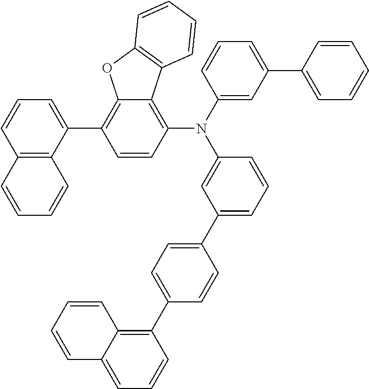 [SMILES: c1ccc(-c2cccc(N(c3cccc(-c4ccc(-c5cccc6ccccc56)cc4)c3)c3ccc(-c4cccc5ccccc45)c4oc5ccccc5c34)c2)cc1] | 21.6 g (73%) | 739.29 |
| | Compound 2-141 | | |
| P50 | 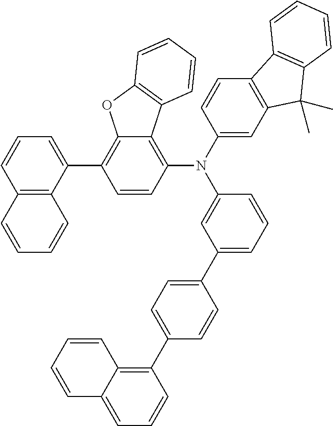 [SMILES: CC1(C)c2ccccc2-c2ccc(N(c3cccc(-c4ccc(-c5cccc6ccccc56)cc4)c3)c3ccc(-c4cccc5ccccc45)c4oc5ccccc5c34)cc21] | 22.2 g (71%) | 779.32 |
| | Compound 2-134 | | |
| P51 | 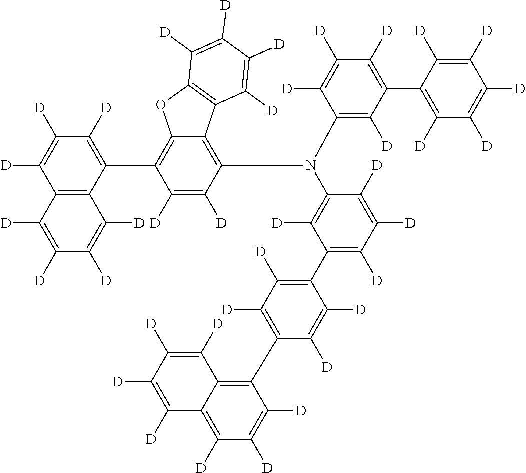 [SMILES: [2H]c1c([2H])c([2H])c(-c2c([2H])c([2H])c([2H])c(N(c3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c2[2H])c([2H])c1[2H]] | 20.5 g (66%) | 776.52 |
| | Compound 2-238 | | |
| P52 | 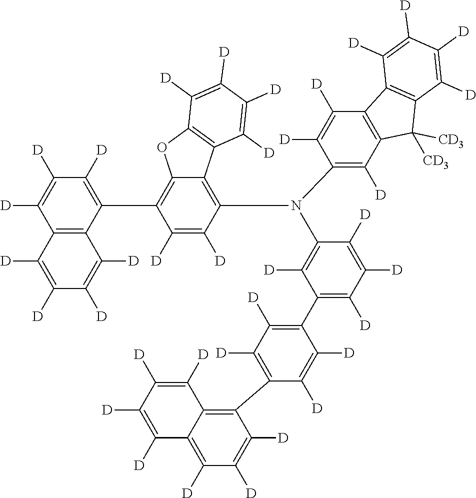 [SMILES: [2H]c1c([2H])c([2H])c2c(c1[2H])-c1c([2H])c([2H])c(N(c3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c1C2(C([2H])([2H])[2H])C([2H])([2H])[2H]] | 204 g (62%) | 820.58 |
| | Compound 2-241 | | |
| P53 | 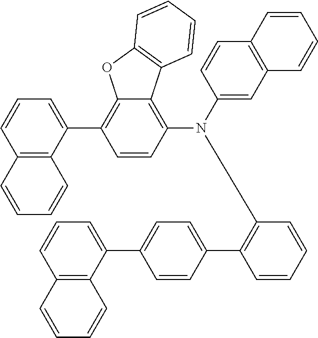 [SMILES: c1ccc(N(c2ccc3ccccc3c2)c2ccc(-c3cccc4ccccc34)c3oc4ccccc4c23)c(-c2ccc(-c3cccc4ccccc34)cc2)c1] | 21.1 g (74%) | 713.27 |
| | Compound 2-244 | | |
| P54 | 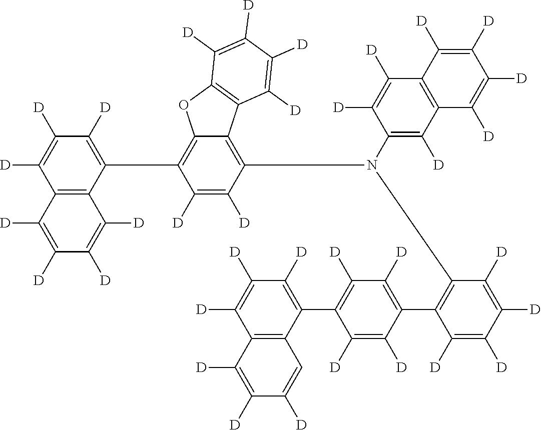 [SMILES: [2H]c1cc2c(-c3c([2H])c([2H])c(-c4c([2H])c([2H])c([2H])c([2H])c4N(c4c([2H])c([2H])c5c([2H])c([2H])c([2H])c([2H])c5c4[2H])c4c([2H])c([2H])c(-c5c([2H])c([2H])c([2H])c6c([2H])c([2H])c([2H])c([2H])c56)c5oc6c([2H])c([2H])c([2H])c([2H])c6c45)c([2H])c3[2H])c([2H])c([2H])c([2H])c2c([2H])c1[2H]] | 20.9 g (70%) | 747.49 |
| | Compound 2-304 | | |
| P55 | 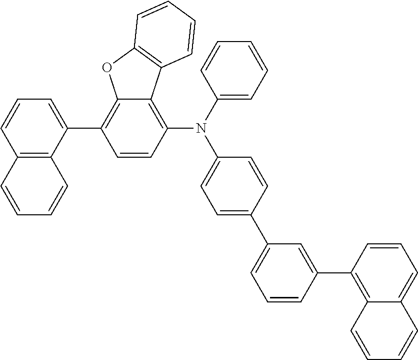 [SMILES: c1ccc(N(c2ccc(-c3cccc(-c4cccc5ccccc45)c3)cc2)c2ccc(-c3cccc4ccccc34)c3oc4ccccc4c23)cc1] | 19.1 g (72%) | 663.26 |
| | Compound 2-308 | | |
| P56 | 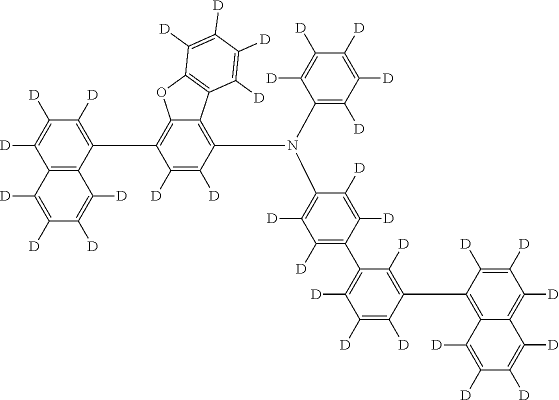 [SMILES: [2H]c1c([2H])c([2H])c(N(c2c([2H])c([2H])c(-c3c([2H])c([2H])c([2H])c(-c4c([2H])c([2H])c([2H])c5c([2H])c([2H])c([2H])c([2H])c45)c3[2H])c([2H])c2[2H])c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H]] | 19.2 g (69%) | 696.46 |
| | Compound 2-350 | | |
| P57 | 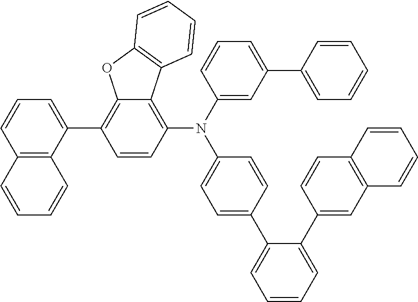 [SMILES: c1ccc(-c2cccc(N(c3ccc(-c4ccccc4-c4ccc5ccccc5c4)cc3)c3ccc(-c4cccc5ccccc45)c4oc5ccccc5c34)c2)cc1] | 19.5 g (66%) | 739.29 |
| | Compound 2-379 | | |
| P58 | 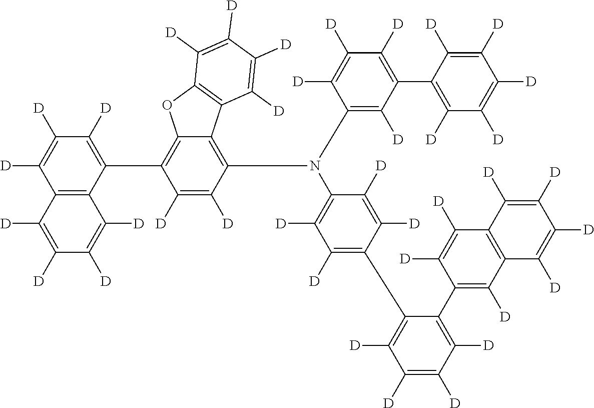 [SMILES: [2H]c1c([2H])c([2H])c(-c2c([2H])c([2H])c([2H])c(N(c3c([2H])c([2H])c(-c4c([2H])c([2H])c([2H])c([2H])c4-c4c([2H])c([2H])c5c([2H])c([2H])c([2H])c([2H])c5c4[2H])c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c2[2H])c([2H])c1[2H]] | 19.9 g (64%) | 776.52 |
| | Compound 2-399 | | |
| P59 | 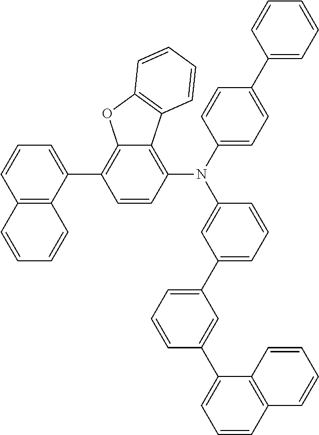 [SMILES: c1ccc(-c2ccc(N(c3cccc(-c4cccc(-c5cccc6ccccc56)c4)c3)c3ccc(-c4cccc5ccccc45)c4oc5ccccc5c34)cc2)cc1] | 19.8 g (67%) | 739.29 |
| | Compound 2-405 | | |
| P60 | 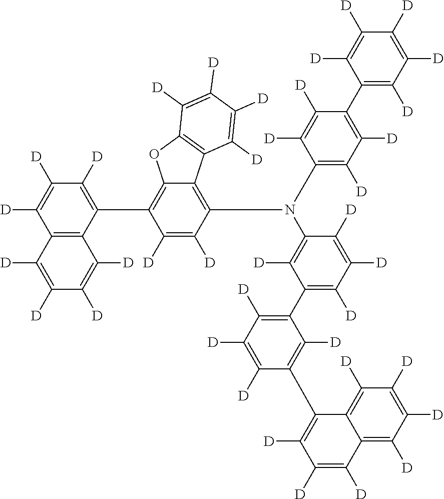 [SMILES: [2H]c1c([2H])c([2H])c(-c2c([2H])c([2H])c(N(c3c([2H])c([2H])c([2H])c(-c4c([2H])c([2H])c([2H])c(-c5c([2H])c([2H])c([2H])c6c([2H])c([2H])c([2H])c([2H])c56)c4[2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H]] | 20.2 g (65%) | 776.52 |
| | Compound 2-434 | | |
| P61 | 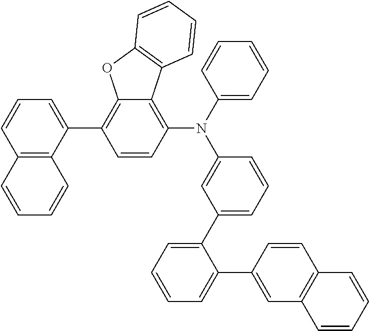 [SMILES: c1ccc(N(c2cccc(-c3ccccc3-c3ccc4ccccc4c3)c2)c2ccc(-c3cccc4ccccc34)c3oc4ccccc4c23)cc1] | 18.3 g (69%) | 663.26 |
| | Compound 2-445 | | |
| P62 | 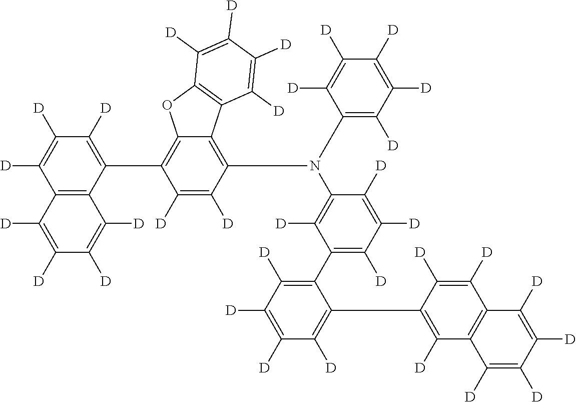 [SMILES: [2H]c1c([2H])c([2H])c(N(c2c([2H])c([2H])c([2H])c(-c3c([2H])c([2H])c([2H])c([2H])c3-c3c([2H])c([2H])c4c([2H])c([2H])c([2H])c([2H])c4c3[2H])c2[2H])c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H]] | 19.0 g (68%) | 696.46 |
| | Compound 2-461 | | |
| P63 | 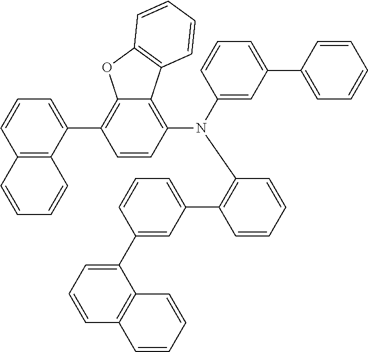 [SMILES: c1ccc(-c2cccc(N(c3ccccc3-c3cccc(-c4cccc5ccccc45)c3)c3ccc(-c4cccc5ccccc45)c4oc5ccccc5c34)c2)cc1] | 18.9 g (64%) | 739.29 |
| | Compound 2-469 | | |
| P64 | 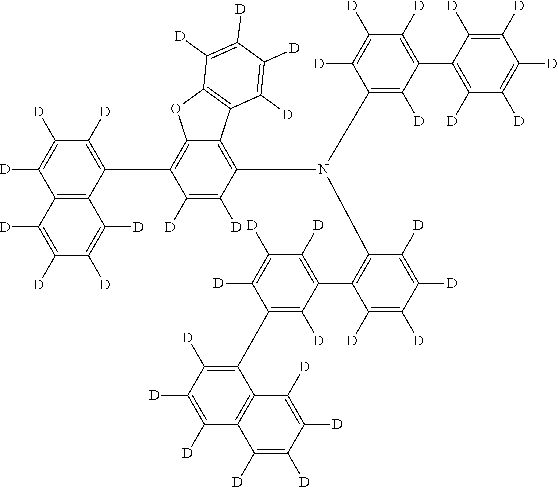 [SMILES: [2H]c1c([2H])c([2H])c(-c2c([2H])c([2H])c([2H])c(N(c3c([2H])c([2H])c([2H])c([2H])c3-c3c([2H])c([2H])c([2H])c(-c4c([2H])c([2H])c([2H])c5c([2H])c([2H])c([2H])c([2H])c45)c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c2[2H])c([2H])c1[2H]] | 19.6 g (63%) | 776.52 |
| | Compound 2-492 | | |
| P65 | 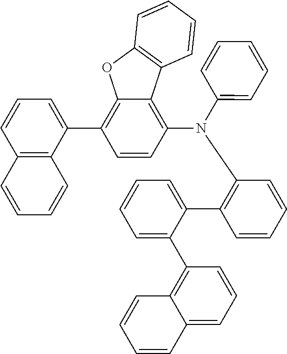 [SMILES: c1ccc(N(c2ccccc2-c2ccccc2-c2cccc3ccccc23)c2ccc(-c3cccc4ccccc34)c3oc4ccccc4c23)cc1] | 16.5 g (62%) | 663.26 |
| | Compound 2-493 | | |
| P66 | 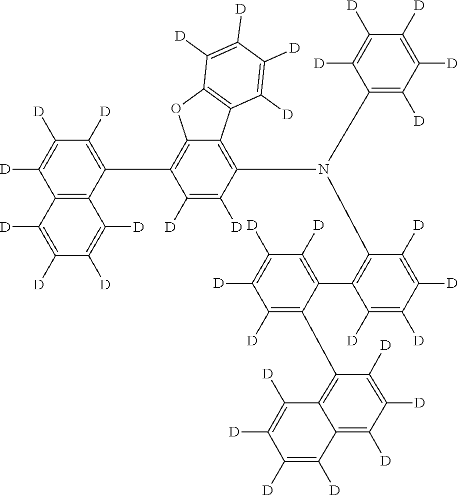 [SMILES: [2H]c1c([2H])c([2H])c(N(c2c([2H])c([2H])c([2H])c([2H])c2-c2c([2H])c([2H])c([2H])c([2H])c2-c2c([2H])c([2H])c([2H])c3c([2H])c([2H])c([2H])c([2H])c23)c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H]] | 17.6 g (63%) | 696.46 |
| | Compound 2-519 | | |
| P67 | 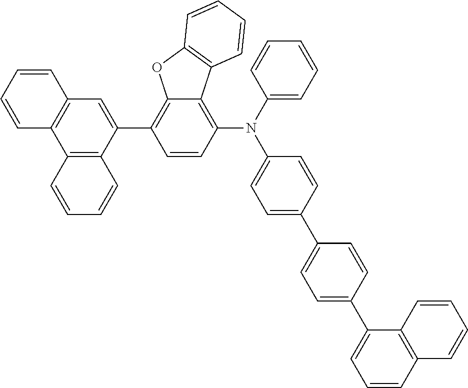 [SMILES: c1ccc(N(c2ccc(-c3ccc(-c4cccc5ccccc45)cc3)cc2)c2ccc(-c3cc4ccccc4c4ccccc34)c3oc4ccccc4c23)cc1] | 21.6 g (75%) | 713.88 |
| | Compound 3-1 | | |
| P68 | 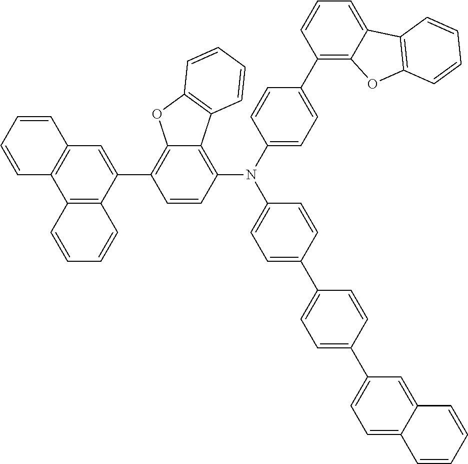 [SMILES: c1ccc2cc(-c3ccc(-c4ccc(N(c5ccc(-c6cccc7c6oc6ccccc67)cc5)c5ccc(-c6cc7ccccc7c7ccccc67)c6oc7ccccc7c56)cc4)cc3)ccc2c1] | 16.9 g (69%) | 880.06 |
| | Compound 3-93 | | |
| P69 | 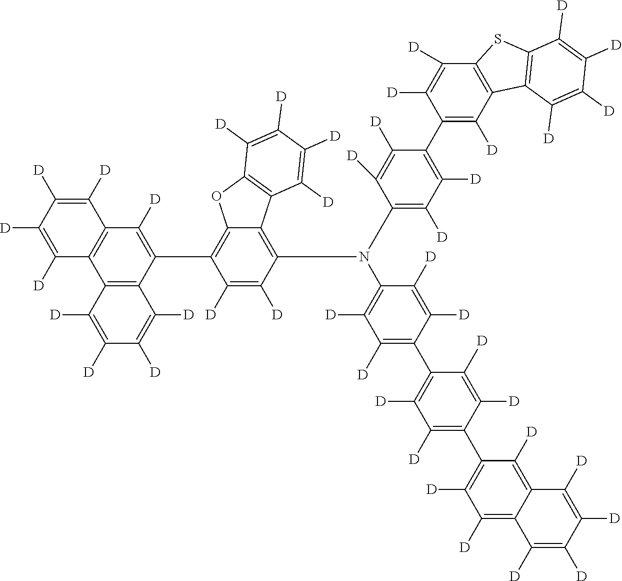 [SMILES: [2H]c1c([2H])c(-c2c([2H])c([2H])c3c([2H])c([2H])c([2H])c([2H])c3c2[2H])c([2H])c([2H])c1-c1c([2H])c([2H])c(N(c2c([2H])c([2H])c(-c3c([2H])c([2H])c4sc5c([2H])c([2H])c([2H])c([2H])c5c4c3[2H])c([2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 17.2 g (71%) | 937.37 |
| | Compound 3-134 | | |
| P70 | 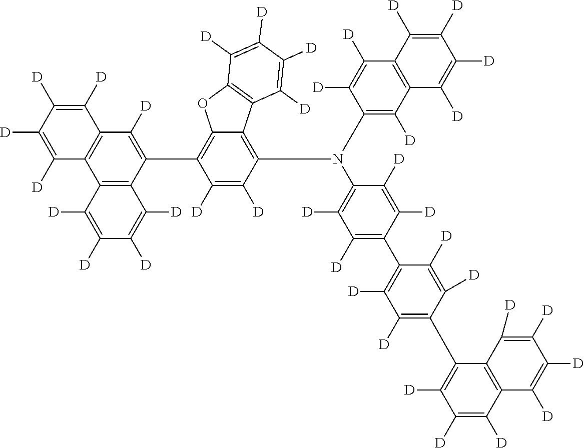 [SMILES: [2H]c1c([2H])c(-c2c([2H])c([2H])c([2H])c3c([2H])c([2H])c([2H])c([2H])c23)c([2H])c([2H])c1-c1c([2H])c([2H])c(N(c2c([2H])c([2H])c3c([2H])c([2H])c([2H])c([2H])c3c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 19.8 g (73%) | 801.17 |
| | Compound 3-131 | | |
| P71 | 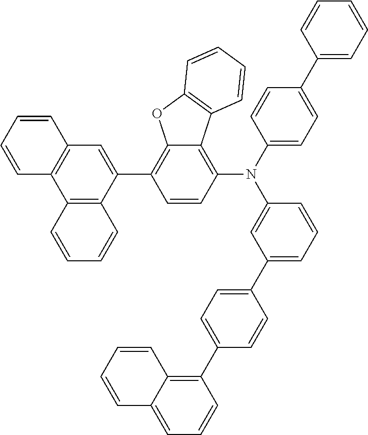 [SMILES: c1ccc(-c2ccc(N(c3cccc(-c4ccc(-c5cccc6ccccc56)cc4)c3)c3ccc(-c4cc5ccccc5c5ccccc45)c4oc5ccccc5c34)cc2)cc1] | 19.6 g (74%) | 789.98 |
| | Compound 3-151 | | |
| P72 | 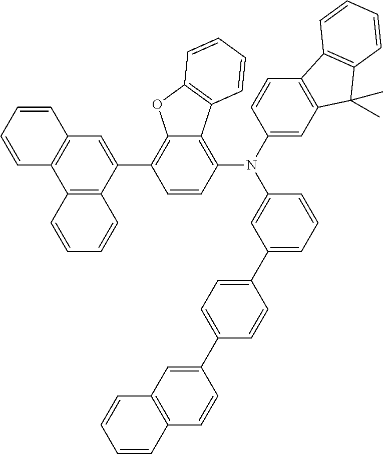 [SMILES: CC1(C)c2ccccc2-c2ccc(N(c3cccc(-c4ccc(-c5ccc6ccccc6c5)cc4)c3)c3ccc(-c4cc5ccccc5c5ccccc45)c4oc5ccccc5c34)cc21] | 18.4 g (72%) | 830.04 |
| | Compound 3-213 | | |
| P73 | 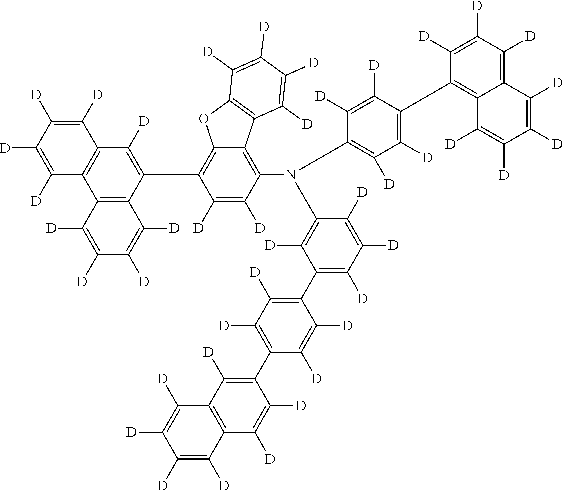 [SMILES: [2H]c1c([2H])c(-c2c([2H])c([2H])c(-c3c([2H])c([2H])c4c([2H])c([2H])c([2H])c([2H])c4c3[2H])c([2H])c2[2H])c([2H])c(N(c2c([2H])c([2H])c(-c3c([2H])c([2H])c([2H])c4c([2H])c([2H])c([2H])c([2H])c34)c([2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c1[2H]] | 17.9 g (71%) | 881.29 |
| | Compound 3-265 | | |
| P74 | 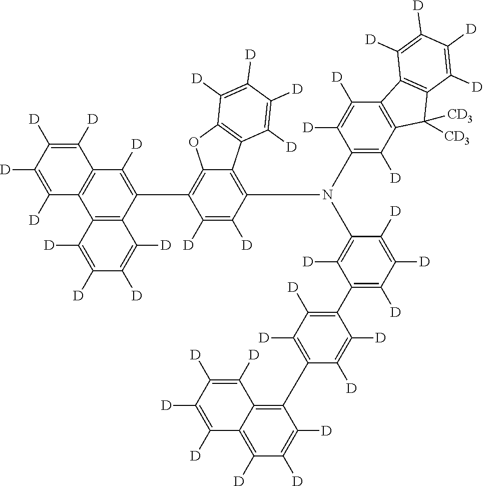 [SMILES: [2H]c1c([2H])c([2H])c2c(c1[2H])-c1c([2H])c([2H])c(N(c3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c([2H])c1C2(C([2H])([2H])[2H])C([2H])([2H])[2H]] | 18.5 g (73%) | 873.31 |
| | Compound 3-268 | | |
| P75 | 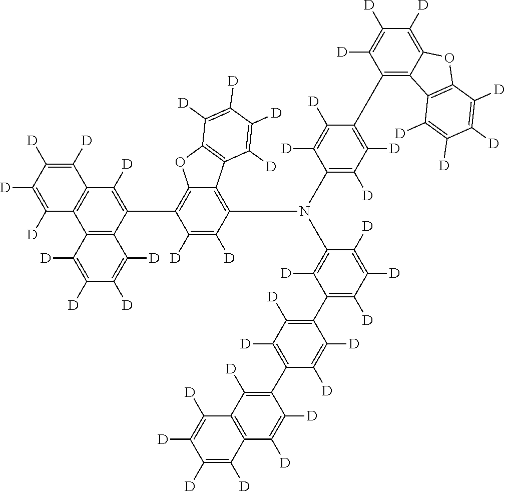 [SMILES: [2H]c1c([2H])c(-c2c([2H])c([2H])c(-c3c([2H])c([2H])c4c([2H])c([2H])c([2H])c([2H])c4c3[2H])c([2H])c2[2H])c([2H])c(N(c2c([2H])c([2H])c(-c3c([2H])c([2H])c([2H])c4oc5c([2H])c([2H])c([2H])c([2H])c5c34)c([2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c1[2H]] | 17.2 g (70%) | 921.31 |
| | Compound 3-271 | | |
| P76 | 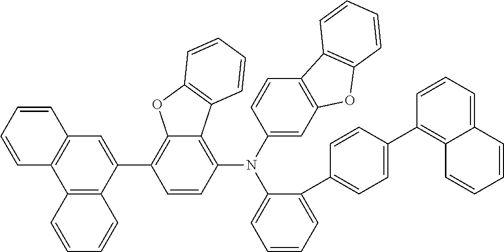 [SMILES: c1ccc(N(c2ccc3c(c2)oc2ccccc23)c2ccc(-c3cc4ccccc4c4ccccc34)c3oc4ccccc4c23)c(-c2ccc(-c3cccc4ccccc34)cc2)c1] | 18.6 g (71%) | 803.96 |
| | Compound 3-281 | | |
| P77 | 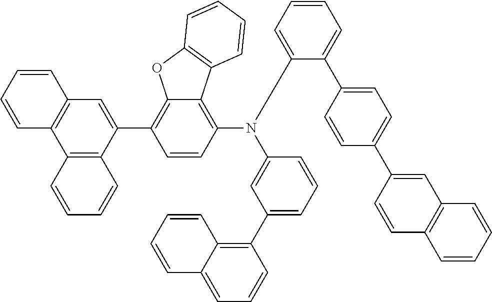 [SMILES: c1cc(-c2cccc3ccccc23)cc(N(c2ccccc2-c2ccc(-c3ccc4ccccc4c3)cc2)c2ccc(-c3cc4ccccc4c4ccccc34)c3oc4ccccc4c23)c1] | 17.7 g (70%) | 840.04 |
| | Compound 3-360 | | |
| P78 | 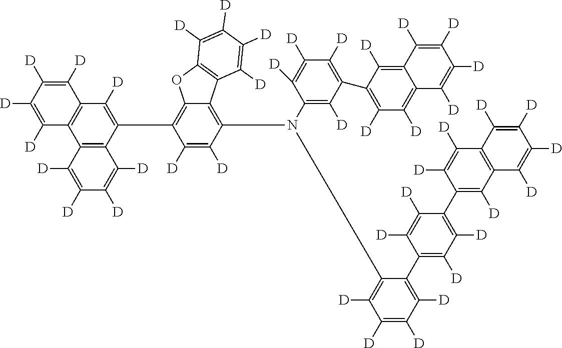 [SMILES: [2H]c1c([2H])c([2H])c(N(c2c([2H])c([2H])c([2H])c(-c3c([2H])c([2H])c4c([2H])c([2H])c([2H])c([2H])c4c3[2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c(-c2c([2H])c([2H])c(-c3c([2H])c([2H])c4c([2H])c([2H])c([2H])c([2H])c4c3[2H])c([2H])c2[2H])c1[2H]] | 18.2 g (72%) | 881.29 |
| | Compound 3-405 | | |
| P79 | 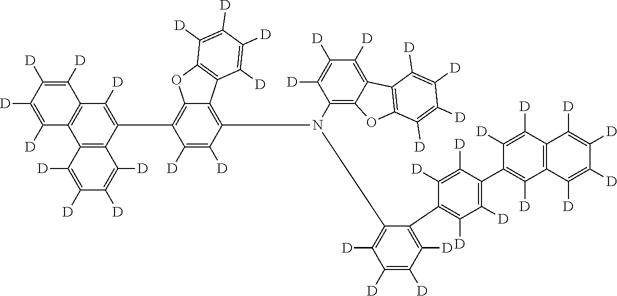 [SMILES: [2H]c1c([2H])c([2H])c(N(c2c([2H])c([2H])c([2H])c3c2oc2c([2H])c([2H])c([2H])c([2H])c23)c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c(-c2c([2H])c([2H])c(-c3c([2H])c([2H])c4c([2H])c([2H])c([2H])c([2H])c4c3[2H])c([2H])c2[2H])c1[2H]] | 18.5 g (71%) | 841.19 |
| | Compound 3-411 | | |
| P80 | 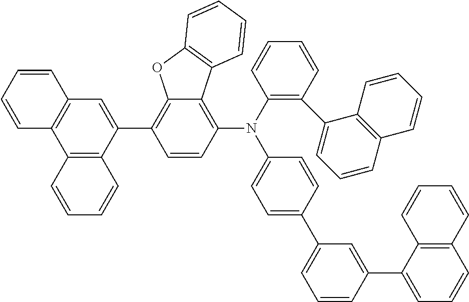 [SMILES: c1cc(-c2ccc(N(c3ccccc3-c3cccc4ccccc34)c3ccc(-c4cc5ccccc5c5ccccc45)c4oc5ccccc5c34)cc2)cc(-c2cccc3ccccc23)c1] | 17.2 g (68%) | 840.04 |
| | Compound 3-427 | | |
| P81 | 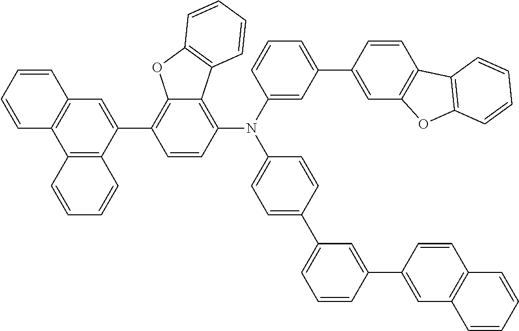 [SMILES: c1cc(-c2ccc(N(c3cccc(-c4ccc5c(c4)oc4ccccc45)c3)c3ccc(-c4cc5ccccc5c5ccccc45)c4oc5ccccc5c34)cc2)cc(-c2ccc3ccccc3c2)c1] | 17.9 g (73%) | 880.06 |
| | Compound 3-462 | | |
| P82 | 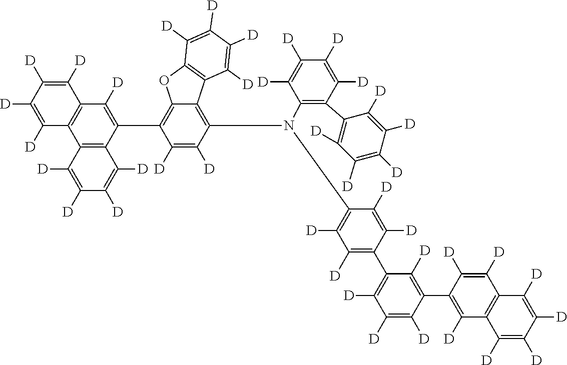 [SMILES: [2H]c1c([2H])c([2H])c(-c2c([2H])c([2H])c([2H])c([2H])c2N(c2c([2H])c([2H])c(-c3c([2H])c([2H])c([2H])c(-c4c([2H])c([2H])c5c([2H])c([2H])c([2H])c([2H])c5c4[2H])c3[2H])c([2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 18.3 g (71%) | 829.22 |
| | Compound 3-470 | | |
| P83 | 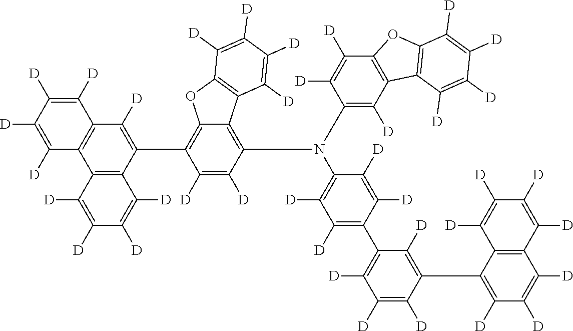 [SMILES: [2H]c1c([2H])c(-c2c([2H])c([2H])c(N(c3c([2H])c([2H])c4oc5c([2H])c([2H])c([2H])c([2H])c5c4c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c([2H])c2[2H])c([2H])c(-c2c([2H])c([2H])c([2H])c3c([2H])c([2H])c([2H])c([2H])c23)c1[2H]] | 21.5 g (72%) | 841.19 |
| | Compound 3-473 | | |
| P84 | 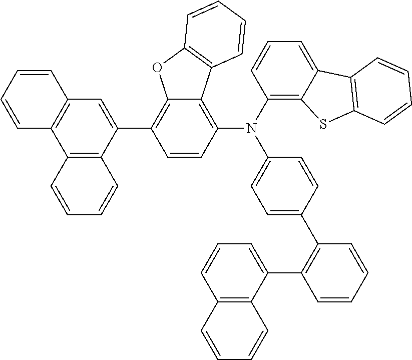 [SMILES: c1ccc(-c2cccc3ccccc23)c(-c2ccc(N(c3cccc4c3sc3ccccc34)c3ccc(-c4cc5ccccc5c5ccccc45)c4oc5ccccc5c34)cc2)c1] | 18.5 g (72%) | 820.02 |
| | Compound 3-480 | | |
| P85 | 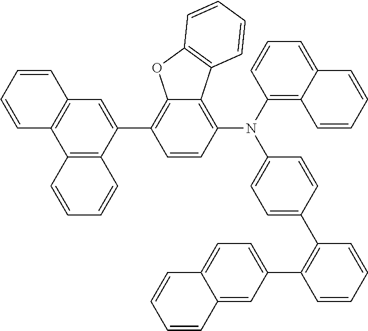 [SMILES: c1ccc(-c2ccc3ccccc3c2)c(-c2ccc(N(c3cccc4ccccc34)c3ccc(-c4cc5ccccc5c5ccccc45)c4oc5ccccc5c34)cc2)c1] | 20.1 g (74%) | 763.94 |
| | Compound 3-496 | | |
| P86 | 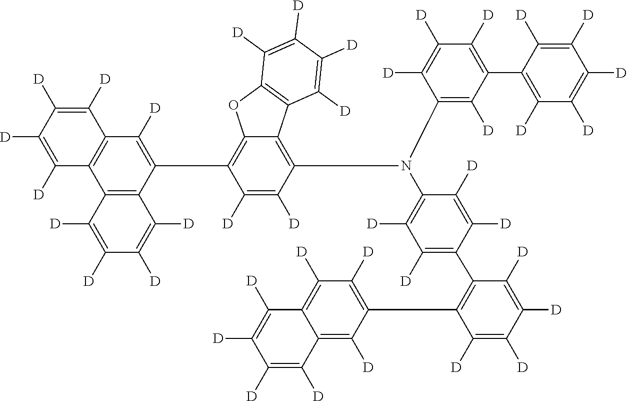 [SMILES: [2H]c1c([2H])c([2H])c(-c2c([2H])c([2H])c([2H])c(N(c3c([2H])c([2H])c(-c4c([2H])c([2H])c([2H])c([2H])c4-c4c([2H])c([2H])c5c([2H])c([2H])c([2H])c([2H])c5c4[2H])c([2H])c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c2[2H])c([2H])c1[2H]] | 18.5 g (70%) | 829.22 |
| | Compound 3-517 | | |
| P87 | 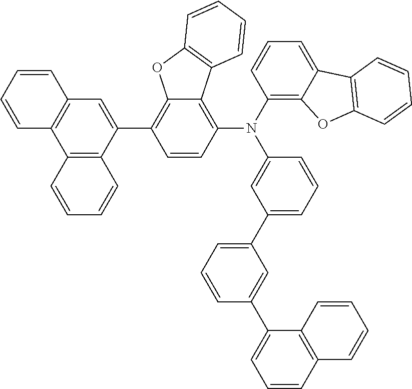 [SMILES: c1cc(-c2cccc(N(c3cccc4c3oc3ccccc34)c3ccc(-c4cc5ccccc5c5ccccc45)c4oc5ccccc5c34)c2)cc(-c2cccc3ccccc23)c1] | 18.3 g (70%) | 803.96 |
| | Compound 3-525 | | |
| P88 | 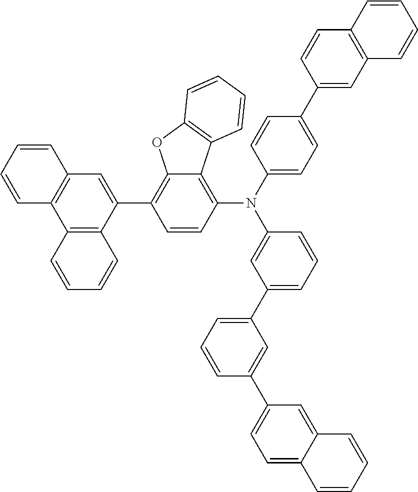 [SMILES: c1cc(-c2cccc(N(c3ccc(-c4ccc5ccccc5c4)cc3)c3ccc(-c4cc5ccccc5c5ccccc45)c4oc5ccccc5c34)c2)cc(-c2ccc3ccccc3c2)c1] | 18.5 g (73%) | 840.04 |
| | Compound 3-547 | | |
| P89 | 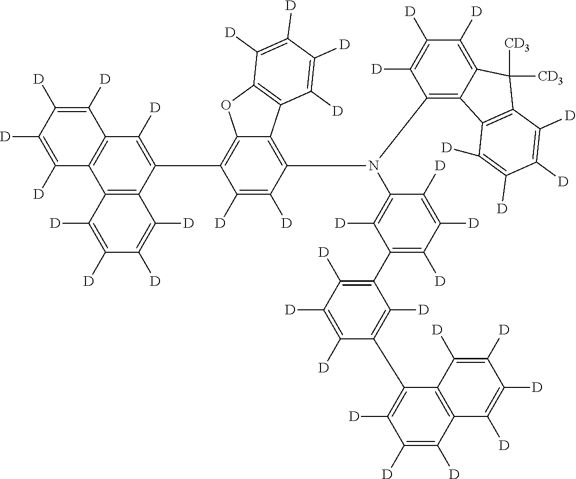 [SMILES: [2H]c1c([2H])c(-c2c([2H])c([2H])c([2H])c(N(c3c([2H])c([2H])c([2H])c4c3-c3c([2H])c([2H])c([2H])c([2H])c3C4(C([2H])([2H])[2H])C([2H])([2H])[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c2[2H])c([2H])c(-c2c([2H])c([2H])c([2H])c3c([2H])c([2H])c([2H])c([2H])c23)c1[2H]] | 18.0 g (71%) | 873.31 |
| | Compound 3-559 | | |
| P90 | 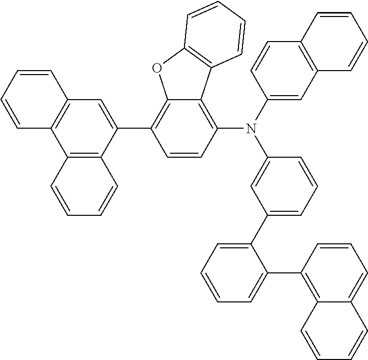 [SMILES: c1cc(-c2ccccc2-c2cccc3ccccc23)cc(N(c2ccc3ccccc3c2)c2ccc(-c3cc4ccccc4c4ccccc34)c3oc4ccccc4c23)c1] | 20.1 g (74%) | 763.94 |
| | Compound 3-565 | | |
| P91 | 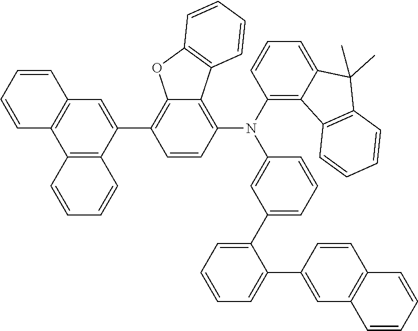 [SMILES: CC1(C)c2ccccc2-c2c(N(c3cccc(-c4ccccc4-c4ccc5ccccc5c4)c3)c3ccc(-c4cc5ccccc5c5ccccc45)c4oc5ccccc5c34)cccc21] | 17.6 g (69%) | 830.04 |
| | Compound 3-586 | | |
| P92 | 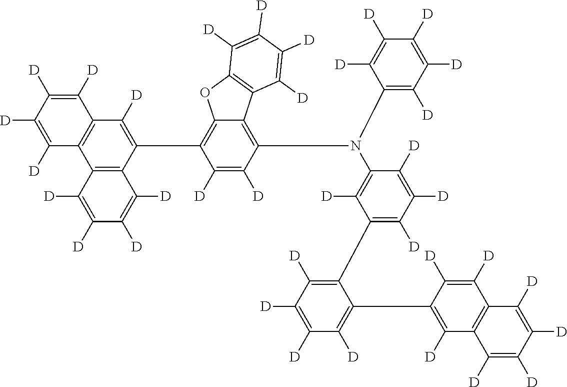 [SMILES: [2H]c1c([2H])c([2H])c(N(c2c([2H])c([2H])c([2H])c(-c3c([2H])c([2H])c([2H])c([2H])c3-c3c([2H])c([2H])c4c([2H])c([2H])c([2H])c([2H])c4c3[2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 20.6 g (73%) | 749.09 |
| | Compound 3-601 | | |
| P93 | 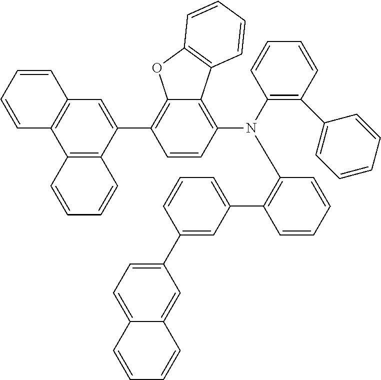 [SMILES: c1ccc(-c2ccccc2N(c2ccccc2-c2cccc(-c3ccc4ccccc4c3)c2)c2ccc(-c3cc4ccccc4c4ccccc34)c3oc4ccccc4c23)cc1] | 18.3 g (69%) | 789.98 |
| | Compound 3-627 | | |
| P94 | 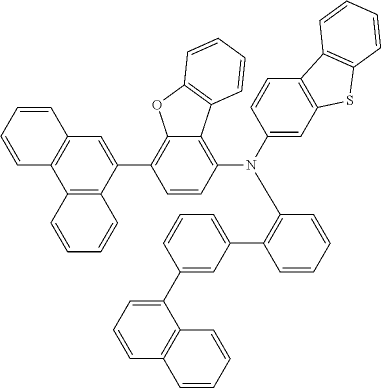 [SMILES: c1cc(-c2ccccc2N(c2ccc3c(c2)sc2ccccc23)c2ccc(-c3cc4ccccc4c4ccccc34)c3oc4ccccc4c23)cc(-c2cccc3ccccc23)c1] | 18.8 g (73%) | 820.02 |
| | Compound 3-608 | | |
| P95 | 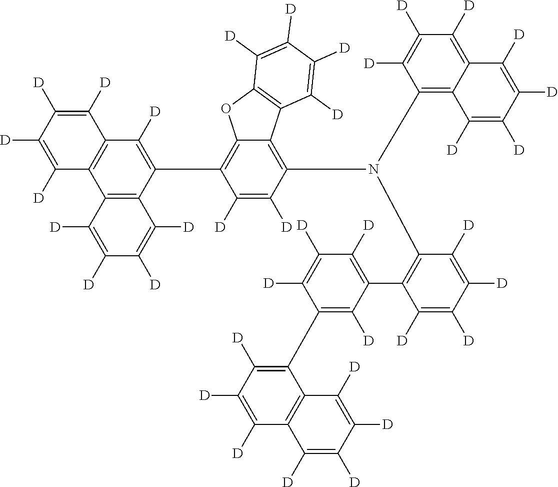 [SMILES: [2H]c1c([2H])c(-c2c([2H])c([2H])c([2H])c([2H])c2N(c2c([2H])c([2H])c([2H])c3c([2H])c([2H])c([2H])c([2H])c23)c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c(-c2c([2H])c([2H])c([2H])c3c([2H])c([2H])c([2H])c([2H])c23)c1[2H]] | 19.5 g (72%) | 801.17 |
| | Compound 3-693 | | |
| P96 | 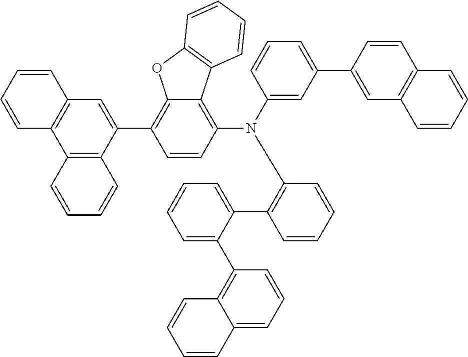 [SMILES: c1cc(-c2ccc3ccccc3c2)cc(N(c2ccccc2-c2ccccc2-c2cccc3ccccc23)c2ccc(-c3cc4ccccc4c4ccccc34)c3oc4ccccc4c23)c1] | 18.0 g (71%) | 840.04 |
| | Compound 3-648 | | |
| P97 | 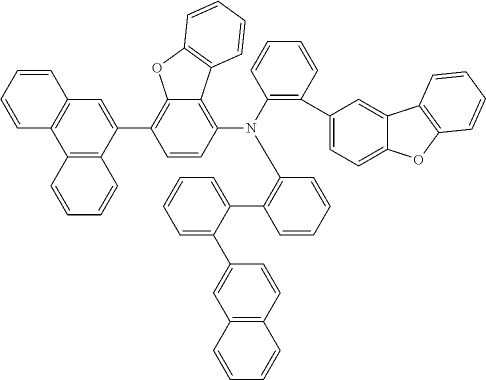 [SMILES: c1ccc(-c2ccccc2N(c2ccccc2-c2ccc3oc4ccccc4c3c2)c2ccc(-c3cc4ccccc4c4ccccc34)c3oc4ccccc4c23)c(-c2ccc3ccccc3c2)c1] | 16.7 g (68%) | 880.06 |
| | Compound 3-672 | | |
| P98 | 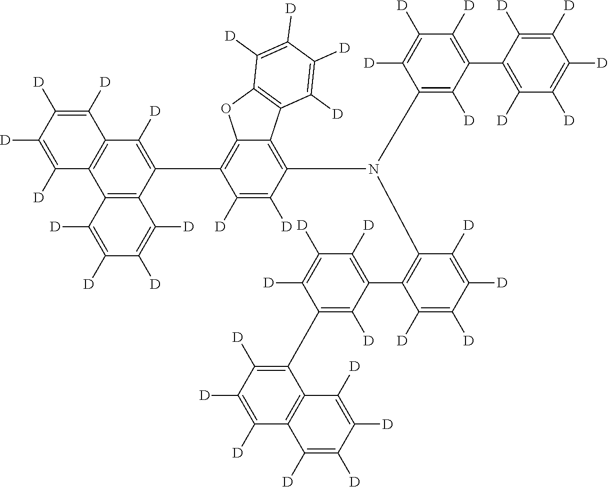 [SMILES: [2H]c1c([2H])c([2H])c(-c2c([2H])c([2H])c([2H])c(N(c3c([2H])c([2H])c([2H])c([2H])c3-c3c([2H])c([2H])c([2H])c(-c4c([2H])c([2H])c([2H])c5c([2H])c([2H])c([2H])c([2H])c45)c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c2[2H])c([2H])c1[2H]] | 19.6 g (70%) | 829.22 |
| | Compound 3-679 | | |
| P99 | 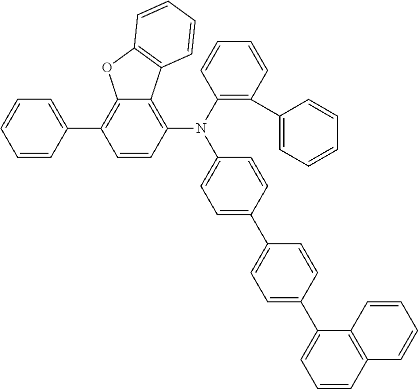 [SMILES: c1ccc(-c2ccccc2N(c2ccc(-c3ccc(-c4cccc5ccccc45)cc3)cc2)c2ccc(-c3ccccc3)c3oc4ccccc4c23)cc1] | 19.0 g (69%) | 689.27 |
| | Compound 1-17 | | |
| P100 | 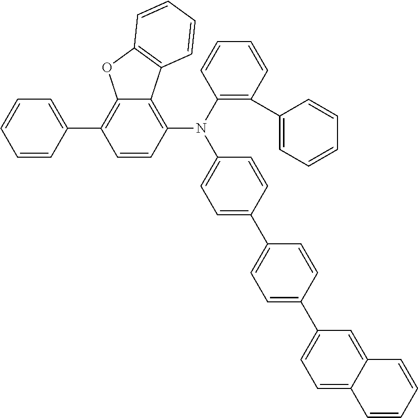 [SMILES: c1ccc(-c2ccccc2N(c2ccc(-c3ccc(-c4ccc5ccccc5c4)cc3)cc2)c2ccc(-c3ccccc3)c3oc4ccccc4c23)cc1] | 18.7 g (68%) | 689.27 |
| | Compound 1-49 | | |
| P101 | 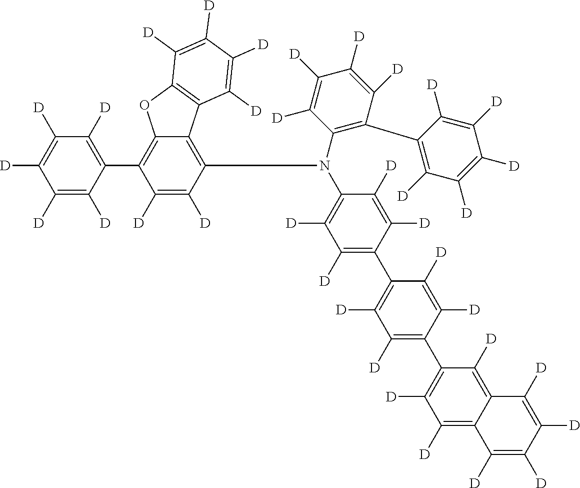 [SMILES: [2H]c1c([2H])c([2H])c(-c2c([2H])c([2H])c([2H])c([2H])c2N(c2c([2H])c([2H])c(-c3c([2H])c([2H])c(-c4c([2H])c([2H])c5c([2H])c([2H])c([2H])c([2H])c5c4[2H])c([2H])c3[2H])c([2H])c2[2H])c2c([2H])c([2H])c(-c3c([2H])c([2H])c([2H])c([2H])c3[2H])c3oc4c([2H])c([2H])c([2H])c([2H])c4c23)c([2H])c1[2H]] | 18.3 g (63%) | 724.49 |
| | Compound 1-79 | | |

### Experimental Example 1] Measurement of HOMO and LUMO

The effect of the compounds of the present disclosure was confirmed through the following experiments, which are provided as representative examples only and are not intended to limit the scope of the experimental examples.

### Experimental Example 1: Simulation Results for Hole Transport Auxiliary Layer

The hole transport auxiliary layer serves to reduce the accumulation of holes at the interface of the emitting layer due to the difference in the HOMO energy levels between the hole transport layer and the emitting layer. For this purpose, it is preferable that the HOMO energy level difference between the hole transport auxiliary layer and the emitting layer be smaller than that between the hole transport auxiliary layer and the hole transport layer. In addition, the hole transport auxiliary layer should have a higher LUMO energy level than that of the emitting layer in order to minimize electron coming from the emitting layer to the hole transport layer.

To confirm whether the organic compound represented by Chemical Formula 1 according to the present disclosure is suitable as a material for the hole transport auxiliary layer, the HOMO energy level (eV) and LUMO energy levels (eV) were calculated using Spartan software (B3LYP DFT 6-31G* by Spartan'16). The results are shown in Table 2 below.

TABLE 2

| | | HOMO | LUMO |
|---|---|---|---|
| | Compound | (calculation) | (calculation) |
| Compounds | Number | (eV) | (eV) |
| 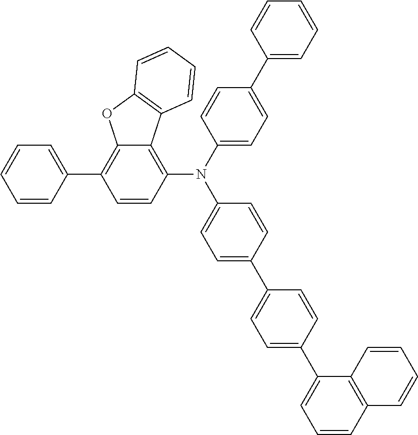 [SMILES: c1ccc(-c2ccc(N(c3ccc(-c4ccc(-c5cccc6ccccc56)cc4)cc3)c3ccc(-c4ccccc4)c4oc5ccccc5c34)cc2)cc1] | 1-9 | −5.01 | −1.18 |
| 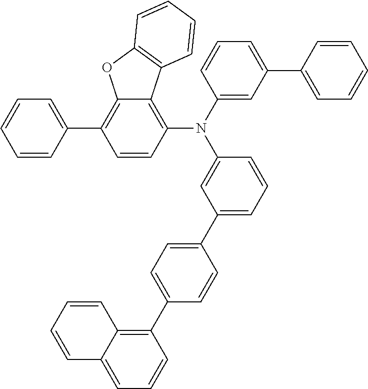 [SMILES: c1ccc(-c2cccc(N(c3cccc(-c4ccc(-c5cccc6ccccc56)cc4)c3)c3ccc(-c4ccccc4)c4oc5ccccc5c34)c2)cc1] | 1-92 | −5.07 | −1.15 |
| 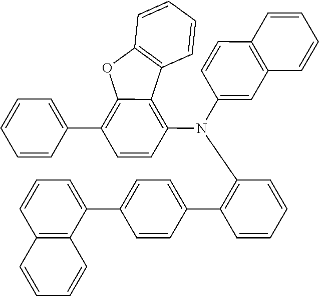 [SMILES: c1ccc(-c2ccc(N(c3ccc4ccccc4c3)c3ccccc3-c3ccc(-c4cccc5ccccc45)cc3)c3c2oc2ccccc23)cc1] | 1-155 | −4.97 | −1.19 |
| 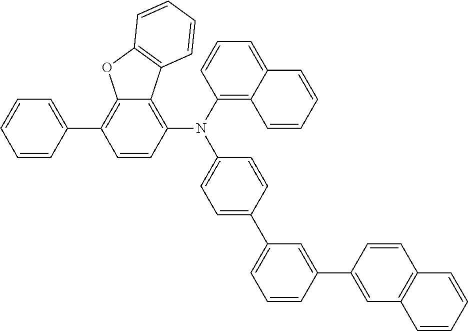 [SMILES: c1ccc(-c2ccc(N(c3ccc(-c4cccc(-c5ccc6ccccc6c5)c4)cc3)c3cccc4ccccc34)c3c2oc2ccccc23)cc1] | 1-257 | −4.97 | −1.32 |
| 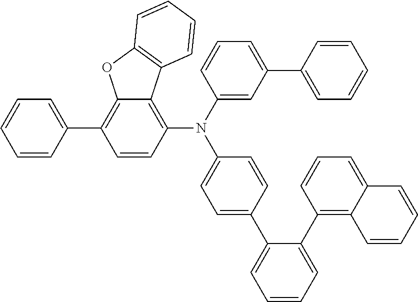 [SMILES: c1ccc(-c2cccc(N(c3ccc(-c4ccccc4-c4cccc5ccccc45)cc3)c3ccc(-c4ccccc4)c4oc5ccccc5c34)c2)cc1] | 1-303 | −5.00 | −1.18 |
| 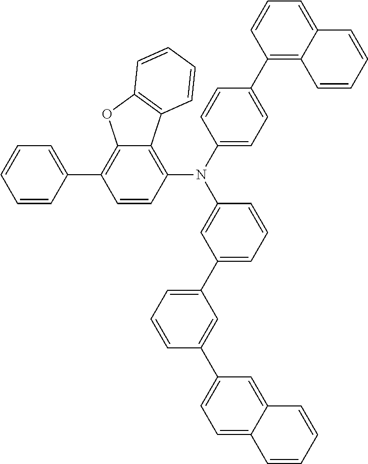 [SMILES: c1ccc(-c2ccc(N(c3ccc(-c4cccc5ccccc45)cc3)c3cccc(-c4cccc(-c5ccc6ccccc6c5)c4)c3)c3c2oc2ccccc23)cc1] | 1-376 | −5.06 | −1.22 |
| 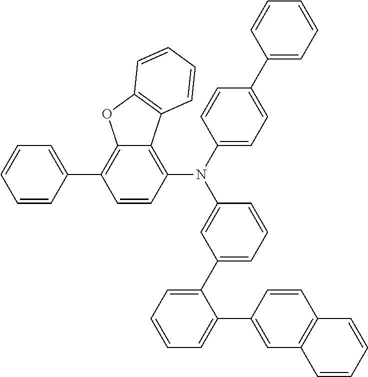 [SMILES: c1ccc(-c2ccc(N(c3cccc(-c4ccccc4-c4ccc5ccccc5c4)c3)c3ccc(-c4ccccc4)c4oc5ccccc5c34)cc2)cc1] | 1-418 | −4.98 | −1.21 |
| 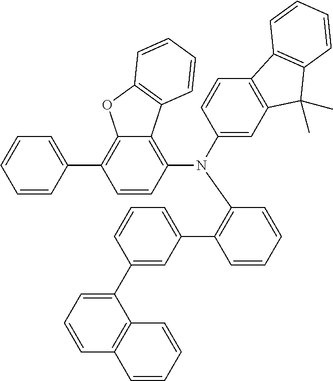 [SMILES: CC1(C)c2ccccc2-c2ccc(N(c3ccccc3-c3cccc(-c4cccc5ccccc45)c3)c3ccc(-c4ccccc4)c4oc5ccccc5c34)cc21] | 1-440 | −4.86 | −1.11 |
| 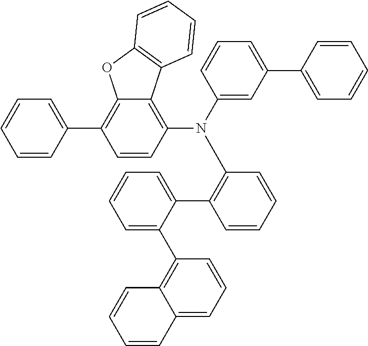 [SMILES: c1ccc(-c2cccc(N(c3ccccc3-c3ccccc3-c3cccc4ccccc34)c3ccc(-c4ccccc4)c4oc5ccccc5c34)c2)cc1] | 1-480 | −5.08 | −1.10 |
| 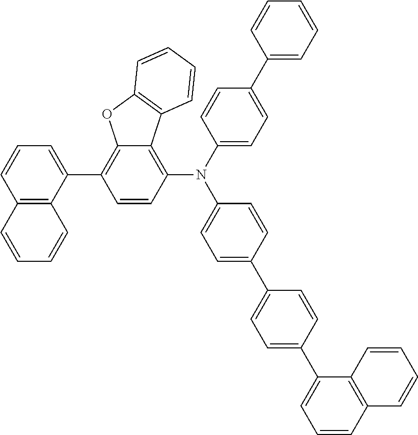 [SMILES: c1ccc(-c2ccc(N(c3ccc(-c4ccc(-c5cccc6ccccc56)cc4)cc3)c3ccc(-c4cccc5ccccc45)c4oc5ccccc5c34)cc2)cc1] | 2-9 | −5.03 | −1.20 |
| 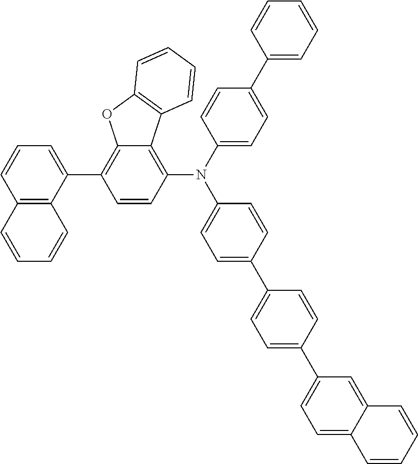 [SMILES: c1ccc(-c2ccc(N(c3ccc(-c4ccc(-c5ccc6ccccc6c5)cc4)cc3)c3ccc(-c4cccc5ccccc45)c4oc5ccccc5c34)cc2)cc1] | 2-41 | −5.02 | −1.24 |
| 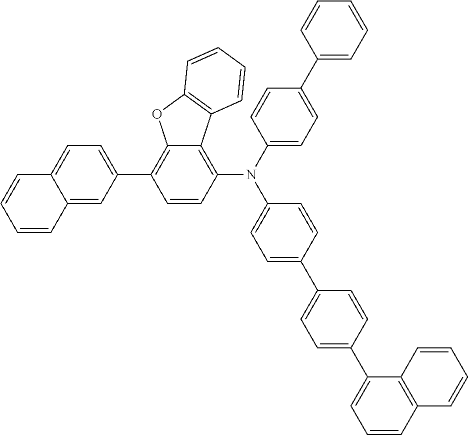 [SMILES: c1ccc(-c2ccc(N(c3ccc(-c4ccc(-c5cccc6ccccc56)cc4)cc3)c3ccc(-c4ccc5ccccc5c4)c4oc5ccccc5c34)cc2)cc1] | 2-73 | −5.01 | −1.33 |
| 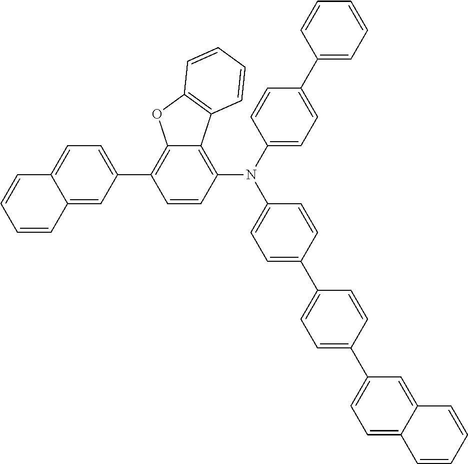 [SMILES: c1ccc(-c2ccc(N(c3ccc(-c4ccc(-c5ccc6ccccc6c5)cc4)cc3)c3ccc(-c4ccc5ccccc5c4)c4oc5ccccc5c34)cc2)cc1] | 2-93 | −5.00 | −1.34 |
| 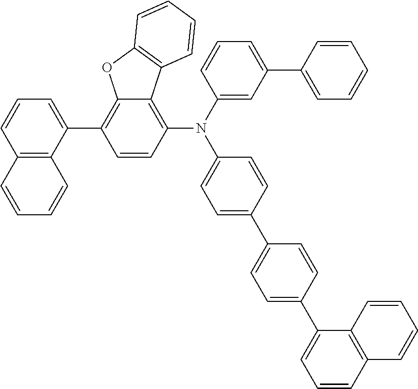 [SMILES: c1ccc(-c2cccc(N(c3ccc(-c4ccc(-c5cccc6ccccc56)cc4)cc3)c3ccc(-c4cccc5ccccc45)c4oc5ccccc5c34)c2)cc1] | 2-13 | −5.03 | −1.29 |
| 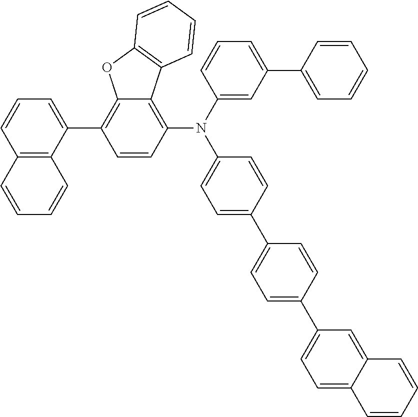 [SMILES: c1ccc(-c2cccc(N(c3ccc(-c4ccc(-c5ccc6ccccc6c5)cc4)cc3)c3ccc(-c4cccc5ccccc45)c4oc5ccccc5c34)c2)cc1] | 2-45 | −5.02 | −1.32 |
| 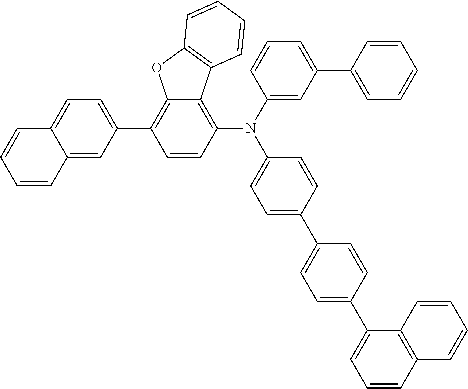 [SMILES: c1ccc(-c2cccc(N(c3ccc(-c4ccc(-c5cccc6ccccc56)cc4)cc3)c3ccc(-c4ccc5ccccc5c4)c4oc5ccccc5c34)c2)cc1] | 2-77 | −5.01 | −1.36 |
| 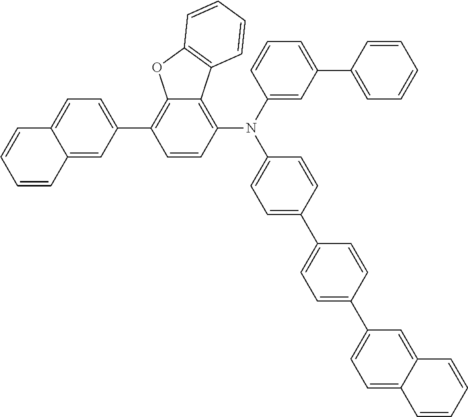 [SMILES: c1ccc(-c2cccc(N(c3ccc(-c4ccc(-c5ccc6ccccc6c5)cc4)cc3)c3ccc(-c4ccc5ccccc5c4)c4oc5ccccc5c34)c2)cc1] | 2-97 | −5.01 | −1.38 |
| 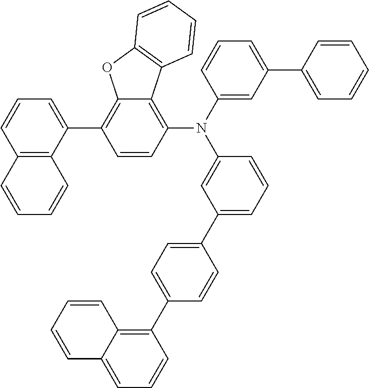 [SMILES: c1ccc(-c2cccc(N(c3cccc(-c4ccc(-c5cccc6ccccc56)cc4)c3)c3ccc(-c4cccc5ccccc45)c4oc5ccccc5c34)c2)cc1] | 2-141 | −5.10 | −1.15 |
| 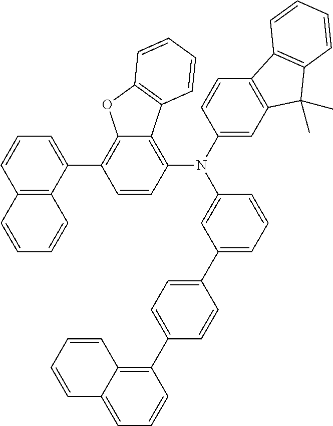 [SMILES: CC1(C)c2ccccc2-c2ccc(N(c3cccc(-c4ccc(-c5cccc6ccccc56)cc4)c3)c3ccc(-c4cccc5ccccc45)c4oc5ccccc5c34)cc21] | 2-134 | −5.01 | −1.15 |
| 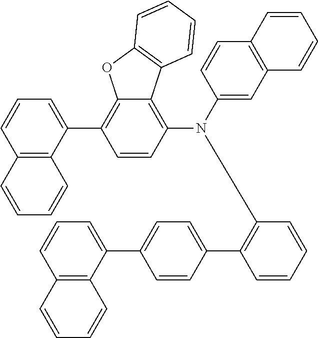 [SMILES: c1ccc(N(c2ccc3ccccc3c2)c2ccc(-c3cccc4ccccc34)c3oc4ccccc4c23)c(-c2ccc(-c3cccc4ccccc34)cc2)c1] | 2-244 | −4.99 | −1.20 |
| 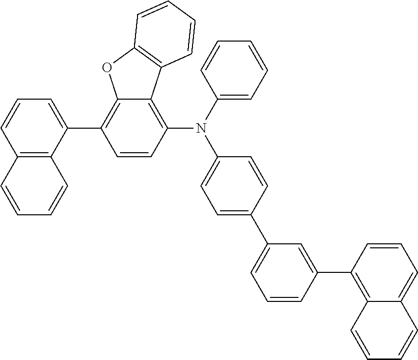 [SMILES: c1ccc(N(c2ccc(-c3cccc(-c4cccc5ccccc45)c3)cc2)c2ccc(-c3cccc4ccccc34)c3oc4ccccc4c23)cc1] | 2-308 | −5.05 | −1.17 |
| 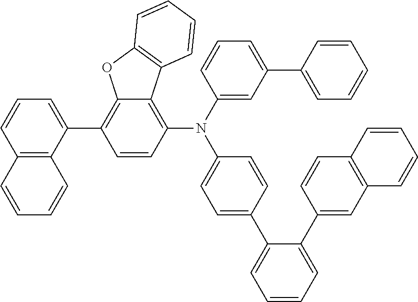 [SMILES: c1ccc(-c2cccc(N(c3ccc(-c4ccccc4-c4ccc5ccccc5c4)cc3)c3ccc(-c4cccc5ccccc45)c4oc5ccccc5c34)c2)cc1] | 2-379 | −5.03 | −1.16 |
| 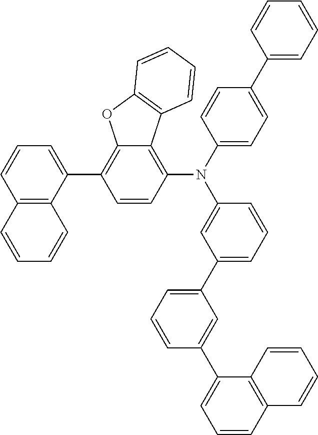 [SMILES: c1ccc(-c2ccc(N(c3cccc(-c4cccc(-c5cccc6ccccc56)c4)c3)c3ccc(-c4cccc5ccccc45)c4oc5ccccc5c34)cc2)cc1] | 2-405 | −5.05 | −1.16 |
| 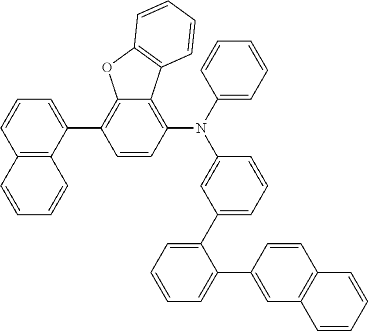 [SMILES: c1ccc(N(c2cccc(-c3ccccc3-c3ccc4ccccc4c3)c2)c2ccc(-c3cccc4ccccc34)c3oc4ccccc4c23)cc1] | 2-445 | −5.00 | −1.22 |
| 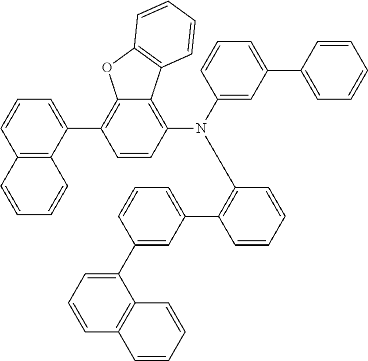 [SMILES: c1ccc(-c2cccc(N(c3ccccc3-c3cccc(-c4cccc5ccccc45)c3)c3ccc(-c4cccc5ccccc45)c4oc5ccccc5c34)c2)cc1] | 2-469 | −5.06 | −1.18 |
| 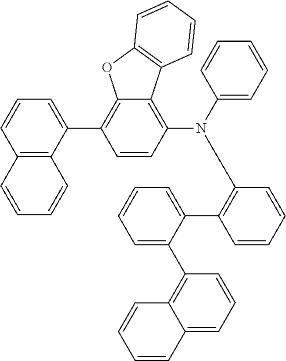 [SMILES: c1ccc(N(c2ccccc2-c2ccccc2-c2cccc3ccccc23)c2ccc(-c3cccc4ccccc34)c3oc4ccccc4c23)cc1] | 2-493 | −4.96 | −1.15 |
| 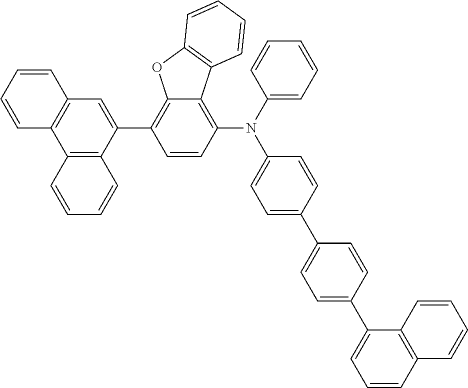 [SMILES: c1ccc(N(c2ccc(-c3ccc(-c4cccc5ccccc45)cc3)cc2)c2ccc(-c3cc4ccccc4c4ccccc34)c3oc4ccccc4c23)cc1] | 3-1 | −5.06 | −1.19 |
| 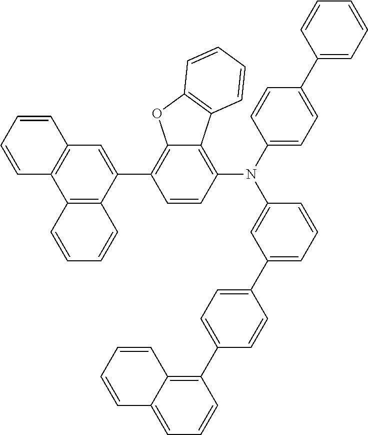 [SMILES: c1ccc(-c2ccc(N(c3cccc(-c4ccc(-c5cccc6ccccc56)cc4)c3)c3ccc(-c4cc5ccccc5c5ccccc45)c4oc5ccccc5c34)cc2)cc1] | 3-151 | −5.07 | −1.19 |
| 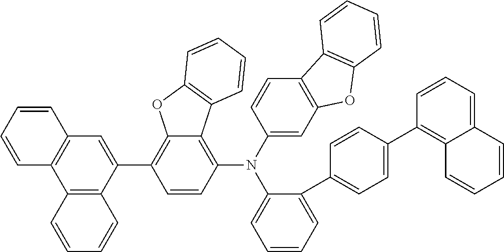 [SMILES: c1ccc(N(c2ccc3c(c2)oc2ccccc23)c2ccc(-c3cc4ccccc4c4ccccc34)c3oc4ccccc4c23)c(-c2ccc(-c3cccc4ccccc34)cc2)c1] | 3-281 | −5.05 | −1.19 |
| 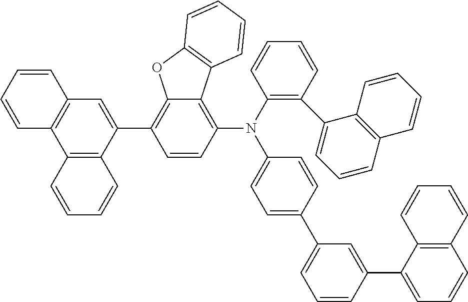 [SMILES: c1cc(-c2ccc(N(c3ccccc3-c3cccc4ccccc34)c3ccc(-c4cc5ccccc5c5ccccc45)c4oc5ccccc5c34)cc2)cc(-c2cccc3ccccc23)c1] | 3-427 | −5.02 | −1.21 |
| 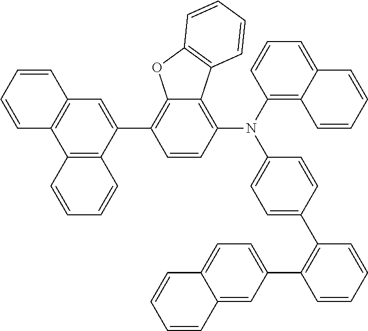 [SMILES: c1ccc(-c2ccc3ccccc3c2)c(-c2ccc(N(c3cccc4ccccc34)c3ccc(-c4cc5ccccc5c5ccccc45)c4oc5ccccc5c34)cc2)c1] | 3-496 | −4.92 | −1.27 |
| 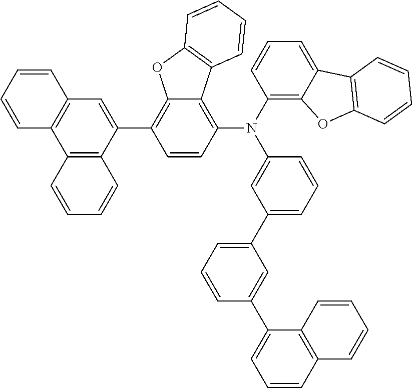 [SMILES: c1cc(-c2cccc(N(c3cccc4c3oc3ccccc34)c3ccc(-c4cc5ccccc5c5ccccc45)c4oc5ccccc5c34)c2)cc(-c2cccc3ccccc23)c1] | 3-525 | −5.06 | −1.17 |
| 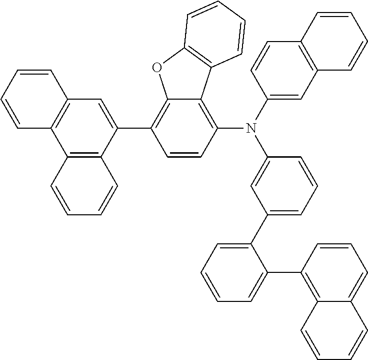 [SMILES: c1cc(-c2ccccc2-c2cccc3ccccc23)cc(N(c2ccc3ccccc3c2)c2ccc(-c3cc4ccccc4c4ccccc34)c3oc4ccccc4c23)c1] | 3-565 | −4.93 | −1.23 |
| 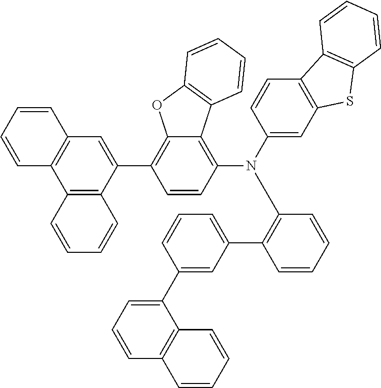 [SMILES: c1cc(-c2ccccc2N(c2ccc3c(c2)sc2ccccc23)c2ccc(-c3cc4ccccc4c4ccccc34)c3oc4ccccc4c23)cc(-c2cccc3ccccc23)c1] | 3-608 | −5.07 | −1.23 |
| 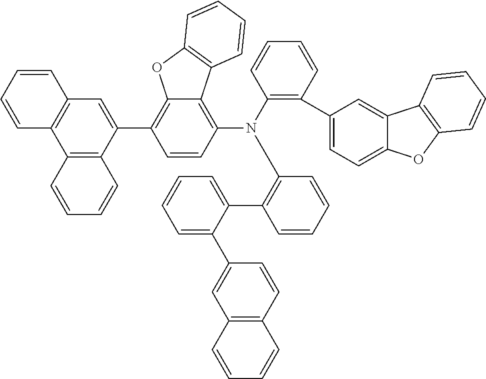 [SMILES: c1ccc(-c2ccccc2N(c2ccccc2-c2ccc3oc4ccccc4c3c2)c2ccc(-c3cc4ccccc4c4ccccc34)c3oc4ccccc4c23)c(-c2ccc3ccccc3c2)c1] | 3-672 | −5.13 | −1.18 |

### [Example 1] Manufacturing of Organic Light Emitting Diode (Blue Emitting Layer)

A substrate on which an ITO (100 nm) serving as the first electrode (anode) of the organic light emitting diode was stacked was patterned by a photolithography process to define the regions of a second electrode (cathode) and a first electrode (anode), and an insulating layer. Subsequently, the surface of the first electrode (ITO) was treated with UV-ozone and O₂:N₂ plasma to enhance its work function and to clean the surface.

Next, on the anode, a mixture of NDP-9 (2-(7-Dicyanomethylene-1,3,4,5,6,8,9,10-octafluoro-7H-pyren-2-ylidene)-malononitrile) and N4,N4,N4′,N4′-tetra([1,1′-biphenyl]-4-yl)-[1,1′-biphenyl]-4,4′-diamine at a weight ratio of 3:97 was vacuum-deposited to form a hole injection layer (HIL) with a thickness of 10 nm. Subsequently, on the hole injection layer (HIL), N4,N4,N4′,N4′-tetra([1,1′-biphenyl]-4-yl)-[1,1′-biphenyl]-4,4′-diamine (N4,N4,N4′,N4′-Tetra([1,1′-biphenyl]-4-yl)-[1,1′-biphenyl]-4,4′-diamine) was vacuum-deposited to form a hole transport layer (HTL) with a thickness of 100 nm, and on the hole transport layer (HTL), a hole transport auxiliary layer with a thickness of 15 nm was formed using Compound 1-45.

On the hole transport auxiliary layer, 9,10-bis(2-naphthyl)anthracene (ADN) as a host and 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB) as a dopant were vacuum-deposited to form a blue emitting layer (EML) with a thickness of 25 nm, wherein a mixing weight ratio of the host:dopant was 97:3. On the blue emitting layer (EML), a mixture of 2-(4-(9,10-di(naphthalene-2-yl)anthracen-2-yl)phenyl)-1-phenyl-1H-benzo[d]imidazole and Liq at a weight ratio of 1:1 was vacuum-deposited to form an electron transport layer (ETL) with a thickness of 25 nm. On the electron transport layer (ETL), an electron injection layer (EIL) with a thickness of 1 nm was vacuum-deposited using Liq, and on the electron injection layer (EIL), a mixture of magnesium and silver at a weight ratio of 1:4 was vacuum-deposited to form a cathode with a thickness of 16 nm. On the cathode, N4,N4′-bis[4-[bis(3-methylphenyl)amino]phenyl]-N4,N4′-diphenyl-[1,1′-biphenyl]-4,4′-diamine (DNTPD) was vacuum-deposited to form a capping layer with a thickness of 60 nm. An organic light emitting diode was manufactured by bonding a seal cap containing a desiccant using a UV-curable adhesive onto the capping layer, thereby forming an encapsulation layer (or protecting layer) to protect the organic light emitting diode from moisture and oxygen in the atmosphere.

### Examples 2 to 132

Organic light emitting diodes of Examples 2 to 132 were manufactured in the same manner as in Example 1, except that Compound 1-45 used as the material for the hole transport auxiliary layer in Example 1 was replaced with the compounds described in Table 3 below.

### Comparative Examples 1 to 8

Organic light emitting diodes of Comparative Examples 1 to 8 were each manufactured in the same manner as in Example 1, except that Compound 1-45 used as the material for the hole transport auxiliary layer in Example 1 was replaced with Compound A, Compound B, Compound C, Compound D, Compound E, Compound F, Compound G, and Compound H below. The structures of Compounds A to H, which were used as materials for the hole transport auxiliary layer in Comparative Examples 1 to 8, are as follow.

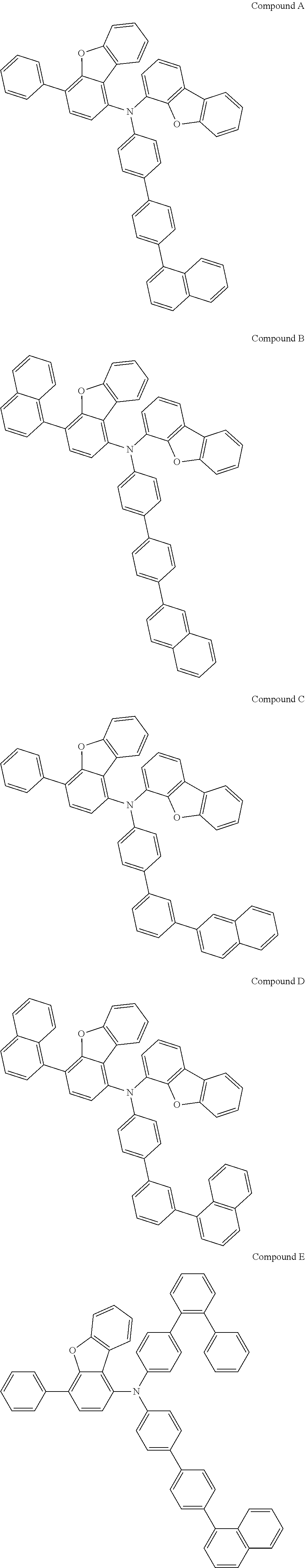 [SMILES: c1ccc(-c2ccc(N(c3ccc(-c4ccc(-c5cccc6ccccc56)cc4)cc3)c3cccc4c3oc3ccccc34)c3c2oc2ccccc23)cc1 ; c1ccc2cc(-c3ccc(-c4ccc(N(c5cccc6c5oc5ccccc56)c5ccc(-c6cccc7ccccc67)c6oc7ccccc7c56)cc4)cc3)ccc2c1 ; c1ccc(-c2ccc(N(c3ccc(-c4cccc(-c5ccc6ccccc6c5)c4)cc3)c3cccc4c3oc3ccccc34)c3c2oc2ccccc23)cc1 ; c1cc(-c2ccc(N(c3cccc4c3oc3ccccc34)c3ccc(-c4cccc5ccccc45)c4oc5ccccc5c34)cc2)cc(-c2cccc3ccccc23)c1 ; c1ccc(-c2ccccc2-c2ccc(N(c3ccc(-c4ccc(-c5cccc6ccccc56)cc4)cc3)c3ccc(-c4ccccc4)c4oc5ccccc5c34)cc2)cc1]

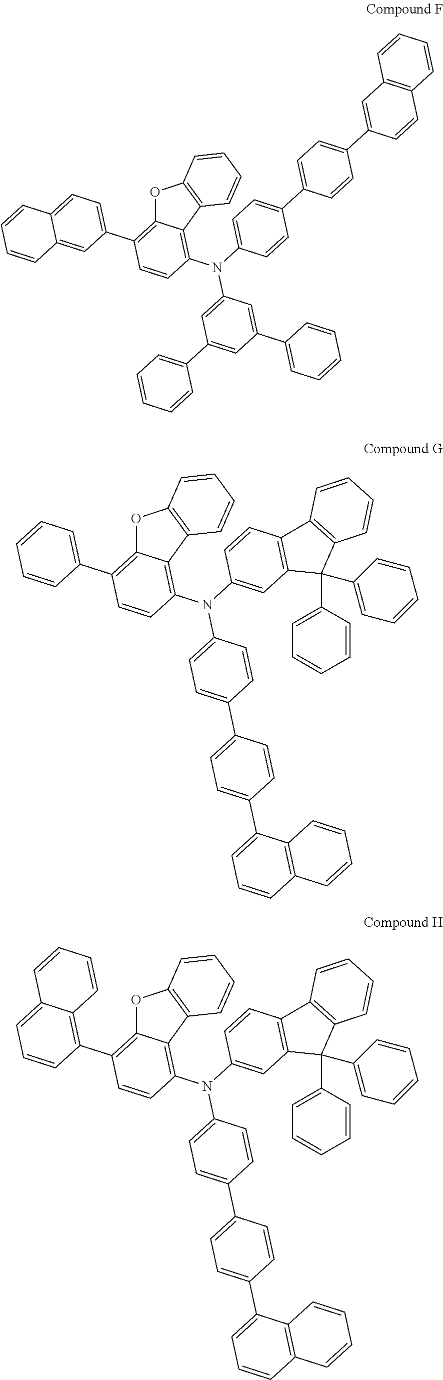 [SMILES: c1ccc(-c2cc(-c3ccccc3)cc(N(c3ccc(-c4ccc(-c5ccc6ccccc6c5)cc4)cc3)c3ccc(-c4ccc5ccccc5c4)c4oc5ccccc5c34)c2)cc1 ; c1ccc(-c2ccc(N(c3ccc(-c4ccc(-c5cccc6ccccc56)cc4)cc3)c3ccc4c(c3)C(c3ccccc3)(c3ccccc3)c3ccccc3-4)c3c2oc2ccccc23)cc1 ; c1ccc(C2(c3ccccc3)c3ccccc3-c3ccc(N(c4ccc(-c5ccc(-c6cccc7ccccc67)cc5)cc4)c4ccc(-c5cccc6ccccc56)c5oc6ccccc6c45)cc32)cc1]

### [Experimental Example 2] Performance Evaluation of Organic Light Emitting Diode (Blue Devices)

For each of the organic light emitting diodes manufactured in Examples 1 to 132 and Comparative Examples 1 to 8, the driving voltage (V) and external quantum efficiency (EQE) (%) were measured by applying a current of 10 mA/cm² using a CS-2000 manufactured by KONICA MINOLTA. In addition, the lifetime (LT95) (hrs) was measured using an M6000 system manufactured by McScience by confirming the time for the luminance to decrease to 95% of the initial luminance under a constant current drive of 10 mA/cm². The measurement results are shown in Table 3 below.

TABLE 3

| | Hole Transport | | External | |
|---|---|---|---|---|
| | Auxiliary | Drive | Quantum | Lifetime |
| | Layer | Voltage | Efficiency | (LT95) |
| Examples | Compounds | (V) | (EQE) (%) | (hrs) |
| Example 1 | 1-13 | 3.48 | 26.8 | 459 |
| Example 2 | 1-14 | 3.47 | 26.7 | 457 |
| Example 3 | 1-26 | 3.46 | 26.5 | 456 |
| Example 4 | 1-28 | 3.46 | 26.4 | 455 |
| Example 5 | 1-45 | 3.48 | 26.9 | 460 |
| Example 6 | 1-46 | 3.47 | 26.8 | 458 |
| Example 7 | 1-58 | 3.46 | 26.8 | 457 |
| Example 8 | 1-60 | 3.46 | 26.6 | 456 |
| Example 9 | 1-76 | 3.48 | 26.9 | 550 |
| Example 10 | 1-149 | 3.49 | 26.6 | 548 |
| Example 11 | 1-92 | 3.49 | 26.6 | 452 |
| Example 12 | 1-94 | 3.48 | 26.5 | 451 |
| Example 13 | 1-105 | 3.47 | 26.4 | 450 |
| Example 14 | 1-124 | 3.49 | 26.6 | 454 |
| Example 15 | 1-126 | 3.48 | 26.5 | 452 |
| Example 16 | 1-137 | 3.47 | 26.4 | 450 |
| Example 17 | 1-165 | 3.50 | 26.4 | 449 |
| Example 18 | 1-180 | 3.48 | 26.3 | 447 |
| Example 19 | 1-197 | 3.50 | 26.5 | 449 |
| Example 20 | 1-212 | 3.48 | 26.4 | 447 |
| Example 21 | 1-239 | 3.50 | 26.1 | 446 |
| Example 22 | 1-269 | 3.49 | 26.2 | 446 |
| Example 23 | 1-9 | 3.63 | 23.6 | 412 |
| Example 24 | 1-11 | 3.62 | 23.5 | 411 |
| Example 25 | 1-22 | 3.61 | 23.4 | 410 |
| Example 26 | 1-41 | 3.63 | 23.7 | 414 |
| Example 27 | 1-43 | 3.62 | 23.6 | 412 |
| Example 28 | 1-54 | 3.61 | 23.5 | 411 |
| Example 29 | 1-73 | 3.63 | 23.7 | 497 |
| Example 30 | 1-89 | 3.63 | 23.4 | 407 |
| Example 31 | 1-103 | 3.62 | 23.3 | 405 |
| Example 32 | 1-121 | 3.63 | 23.5 | 409 |
| Example 33 | 1-135 | 3.62 | 23.4 | 406 |
| Example 34 | 1-163 | 3.64 | 23.2 | 402 |
| Example 35 | 1-174 | 3.63 | 23.1 | 401 |
| Example 36 | 1-193 | 3.65 | 23.3 | 404 |
| Example 37 | 1-235 | 3.64 | 23.1 | 398 |
| Example 38 | 1-264 | 3.66 | 23.1 | 400 |
| Example 39 | 1-279 | 3.64 | 22.9 | 399 |
| Example 40 | 1-17 | 3.76 | 20.3 | 368 |
| Example 41 | 1-18 | 3.75 | 20.2 | 366 |
| Example 42 | 1-49 | 3.76 | 20.4 | 370 |
| Example 43 | 1-50 | 3.75 | 20.3 | 367 |
| Example 44 | 1-64 | 3.74 | 20.2 | 365 |
| Example 45 | 1-98 | 3.75 | 20.1 | 363 |
| Example 46 | 1-109 | 3.74 | 20.0 | 360 |
| Example 47 | 1-128 | 3.76 | 20.3 | 364 |
| Example 48 | 1-169 | 3.77 | 20.1 | 359 |
| Example 49 | 1-184 | 3.75 | 19.9 | 356 |
| Example 50 | 1-202 | 3.76 | 20.1 | 357 |
| Example 51 | 1-254 | 3.75 | 19.7 | 354 |
| Example 52 | 1-272 | 3.77 | 19.8 | 355 |
| Example 53 | 1-1 | 3.88 | 17.0 | 331 |
| Example 54 | 1-6 | 3.86 | 16.9 | 329 |
| Example 55 | 1-34 | 3.87 | 17.0 | 331 |
| Example 56 | 1-82 | 3.87 | 16.9 | 328 |
| Example 57 | 1-119 | 3.86 | 16.8 | 327 |
| Example 58 | 1-155 | 3.88 | 16.4 | 325 |
| Example 59 | 1-185 | 3.89 | 16.5 | 326 |
| Example 60 | 1-226 | 3.9 | 16.2 | 324 |
| Example 61 | 2-13 | 3.43 | 25.2 | 436 |
| Example 62 | 2-15 | 3.44 | 25.1 | 433 |
| Example 63 | 2-45 | 3.44 | 25.4 | 437 |
| Example 64 | 2-77 | 3.43 | 25.3 | 435 |
| Example 65 | 2-79 | 3.45 | 25.1 | 432 |
| Example 66 | 2-97 | 3.44 | 25.2 | 434 |
| Example 67 | 2-98 | 3.45 | 25.0 | 431 |
| Example 68 | 2-119 | 3.41 | 25.8 | 523 |
| Example 69 | 2-122 | 3.40 | 25.9 | 524 |
| Example 70 | 2-125 | 3.42 | 25.8 | 522 |
| Example 71 | 2-128 | 3.43 | 25.7 | 521 |
| Example 72 | 2-141 | 3.44 | 25.7 | 429 |
| Example 73 | 2-143 | 3.45 | 25.1 | 425 |
| Example 74 | 2-173 | 3.43 | 25.7 | 430 |
| Example 75 | 2-175 | 3.44 | 25.4 | 426 |
| Example 76 | 2-205 | 3.43 | 25.5 | 428 |
| Example 77 | 2-225 | 3.44 | 25.3 | 427 |
| Example 78 | 2-238 | 3.41 | 25.8 | 515 |
| Example 79 | 2-273 | 3.42 | 25.6 | 424 |
| Example 80 | 2-275 | 3.44 | 25.1 | 422 |
| Example 81 | 2-287 | 3.43 | 25.4 | 423 |
| Example 82 | 2-329 | 3.43 | 25 | 421 |
| Example 83 | 2-331 | 3.44 | 24.9 | 420 |
| Example 84 | 2-9 | 3.58 | 22.2 | 392 |
| Example 85 | 2-41 | 3.57 | 22.3 | 393 |
| Example 86 | 2-73 | 3.57 | 22.1 | 390 |
| Example 87 | 2-93 | 3.56 | 21.9 | 389 |
| Example 88 | 2-107 | 3.56 | 22.5 | 470 |
| Example 89 | 2-110 | 3.55 | 22.6 | 472 |
| Example 90 | 2-113 | 3.56 | 22.4 | 468 |
| Example 91 | 2-116 | 3.57 | 22.3 | 467 |
| Example 92 | 2-137 | 3.58 | 21.9 | 382 |
| Example 93 | 2-169 | 3.57 | 22.1 | 383 |
| Example 94 | 2-171 | 3.59 | 21.8 | 379 |
| Example 95 | 2-201 | 3.58 | 22.0 | 381 |
| Example 96 | 2-269 | 3.58 | 21.9 | 378 |
| Example 97 | 2-271 | 3.59 | 21.7 | 376 |
| Example 98 | 2-286 | 3.58 | 21.7 | 374 |
| Example 99 | 2-327 | 3.60 | 21.6 | 373 |
| Example 100 | 2-17 | 3.71 | 19.1 | 353 |
| Example 101 | 2-19 | 3.71 | 19.0 | 349 |
| Example 102 | 2-49 | 3.70 | 19.3 | 353 |
| Example 103 | 2-50 | 3.73 | 18.7 | 350 |
| Example 104 | 2-51 | 3.72 | 18.9 | 351 |
| Example 105 | 2-81 | 3.7 | 19.1 | 352 |
| Example 106 | 2-82 | 3.73 | 18.8 | 348 |
| Example 107 | 2-177 | 3.71 | 18.7 | 347 |
| Example 108 | 2-179 | 3.72 | 18.5 | 345 |
| Example 109 | 2-209 | 3.72 | 18.6 | 346 |
| Example 110 | 2-210 | 3.71 | 18.4 | 343 |
| Example 111 | 2-288 | 3.72 | 18.5 | 342 |
| Example 112 | 2-299 | 3.73 | 18.3 | 340 |
| Example 113 | 2-33 | 3.81 | 16.0 | 323 |
| Example 114 | 2-38 | 3.83 | 15.7 | 321 |
| Example 115 | 2-68 | 3.84 | 15.8 | 322 |
| Example 116 | 2-161 | 3.81 | 15.5 | 319 |
| Example 117 | 2-162 | 3.83 | 15.6 | 320 |
| Example 118 | 2-193 | 3.82 | 15.4 | 318 |
| Example 119 | 2-261 | 3.82 | 15.3 | 317 |
| Example 120 | 2-308 | 3.85 | 15.2 | 316 |
| Example 121 | 3-21 | 3.53 | 24.7 | 417 |
| Example 122 | 3-85 | 3.53 | 24.8 | 418 |
| Example 123 | 3-23 | 3.52 | 24.5 | 415 |
| Example 124 | 3-86 | 3.52 | 24.6 | 416 |
| Example 125 | 3-17 | 3.68 | 21.3 | 370 |
| Example 126 | 3-39 | 3.67 | 21.1 | 368 |
| Example 127 | 3-217 | 3.69 | 20.3 | 369 |
| Example 128 | 3-225 | 3.79 | 18.0 | 338 |
| Example 129 | 3-335 | 3.78 | 17.6 | 336 |
| Example 130 | 3-365 | 3.79 | 17.4 | 337 |
| Example 131 | 3-412 | 3.91 | 15.1 | 315 |
| Example 132 | 3-678 | 3.53 | 24.7 | 501 |
| Comp. | Compound A | 4.03 | 12.8 | 210 |
| Example 1 | | | | |
| Comp. | Compound B | 4.04 | 11.9 | 207 |
| Example 2 | | | | |
| Comp. | Compound C | 4.05 | 11.6 | 199 |
| Example 3 | | | | |
| Comp. | Compound D | 4.07 | 11.1 | 190 |
| Example 4 | | | | |
| Comp. | Compound E | 4.06 | 10.9 | 185 |
| Example 5 | | | | |
| Comp. | Compound F | 4.06 | 10.8 | 174 |
| Example 6 | | | | |
| Comp. | Compound G | 4.10 | 10.5 | 170 |
| Example 7 | | | | |
| Comp. | Compound H | 4.12 | 10.4 | 165 |
| Example 8 | | | | |

The organic compounds represented by Chemical Formula 1 according to the present disclosure are characterized by comprising phenyl, naphthyl, and phenanthryl structures in addition to a dibenzofuran bonded to an arylamine and a phenylene-phenylene-naphthyl structure. Compounds A to D (Comparative Examples 1 to 4) in which Ar₁ is a heteroaryl group, compounds E and F (Comparative Examples 5 and 6) in which Ar₁ is a terphenyl group, and compounds G and H (Comparative Examples 7 and 8) in which Ar₁ is a diphenylfluorene group, were found to exhibit degraded organic light emitting diode performance compared to the compounds represented by Chemical Formula 1 according to the present disclosure when such substituents are included.

Due to these characteristic structural features, the organic compounds represented by Chemical Formula 1 according to the present disclosure are capable of regulating hole transporting properties, thereby reducing the accumulation of holes at the interface between the hole transporting auxiliary layer and the emitting layer, compared to the comparative compounds that do not satisfy the structure of Chemical Formula 1. This effect may reduce the quenching phenomenon where excitons are quenched by polarons at the interface of the hole transport auxiliary layer and the emitting layer. As shown in Table 3, it was confirmed that the compounds according to the present disclosure can reduce device degradation and improve device stability compared to the compounds of the Comparative Examples, thereby lowering the driving voltage and improving the efficiency and lifetime when applied to a device.

While the embodiments of the present specification have been described in detail above, the present specification is not necessarily limited to these embodiments, and various modifications may be made without departing from the technical spirit of this specification.

Accordingly, the embodiments disclosed in the present specification are for the purpose of illustration and not for limitation of the technical spirit of the present specification, and the scope of the technical spirit of the present specification is not limited by these embodiments. Therefore, the embodiments described above should be understood as illustrative in all aspects and not as limiting.

What is claimed is:

1. An organic compound represented by the following Chemical Formula 1: 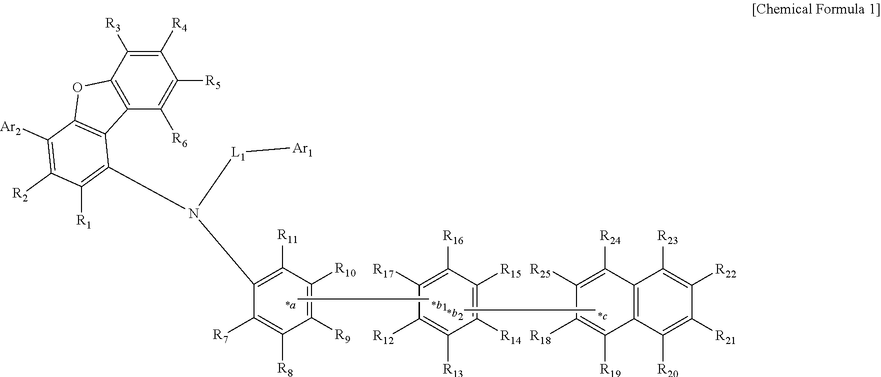
wherein in chemical formula 1,
L₁ is selected from the group consisting of a single bond, a substituted or unsubstituted arylene group having 6 to 30 carbon atoms, and a substituted or unsubstituted heteroarylene group having 1 to 30 carbon atoms;
Ar₁ and Ar₂ are identical to or different from each other, and are each independently selected from the group consisting of a substituted or unsubstituted aryl group having 6 to 30 carbon atoms, and a substituted or unsubstituted heteroaryl group having 1 to 30 carbon atoms;
R₁ to R₂₅ are identical to or different from each other, and are each independently selected from hydrogen, deuterium, a substituted or unsubstituted aryl group having 6 to 30 carbon atoms, and a substituted or unsubstituted heteroaryl group having 1 to 30 carbon atoms;
when L₁, Ar₁, Ar₂, and R₁ to R₂₅ are substituted, the substituents are identical to or different from each other, and may each independently be one or more selected from the group consisting of deuterium, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, a heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms, wherein when a plurality of the substituents are present, the substituents are identical to or different from each other;
*a, *b₁, and *b₂ are different from each other, and represent bonding positions on a phenylene moiety, and *c represents a bonding position on a naphthyl moiety;
one of R₇ to R₁₁ is a single bond that is bonded to *a;
one of R₁₂ to R₁₇ is a single bond that is bonded to *b₁;
one of R₁₂ to R₁₇ is a single bond that is bonded to *b₂; and
one of R₁₈ to R₂₅ is a single bond that is bonded to *c.

2. The organic compound of claim 1, wherein L₁ is selected from the group consisting of a single bond, and a substituted or unsubstituted arylene group having 6 to 15 carbon atoms.

3. The organic compound of claim 1, wherein Ar₁ is selected from the group consisting of a substituted or unsubstituted aryl group having 6 to 30 carbon atoms, and a substituted or unsubstituted heteroaryl group having 1 to 30 carbon atoms and containing at least one heteroatom selected from the group consisting of oxygen (O) and sulfur (S).

4. The organic compound of claim 1, wherein Ar₁ is selected from one of the following Chemical Formulas 18 to 21: 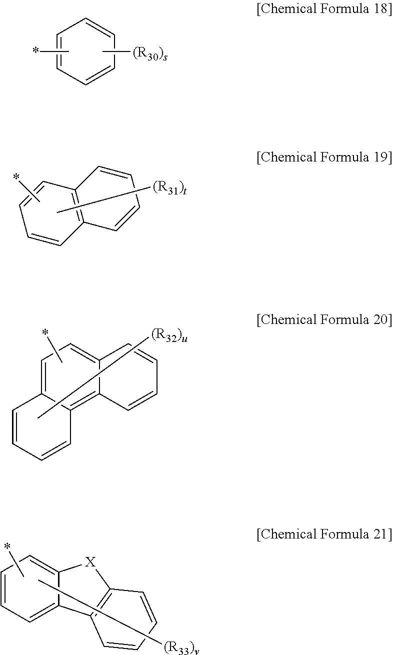
wherein in chemical formulas 18 to 21,
R₃₀ to R₃₃ are identical to or different from each other, and are each independently selected from the group consisting of hydrogen, deuterium, a substituted or unsubstituted aryl group having 6 to 30 carbon atoms, and a substituted or unsubstituted heteroaryl group having 1 to 30 carbon atoms;
s is an integer from 1 to 5, each of t and v is integer from 1 to 7, and u is an integer from 1 to 9;
X is selected from CR₃₄R₃₅, O, and S;
R₃₄ and R₃₅ are identical to or different from each other, and are each independently selected from the group consisting of hydrogen, deuterium, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms; and
* denotes a bonding position.

5. The organic compound of claim 1, wherein Ar₂ is selected from a substituted or unsubstituted aryl group having 6 to 15 carbon atoms.

6. The organic compound of claim 1, wherein Ar₂ is selected from one of the following Chemical Formulas 2 to 5: 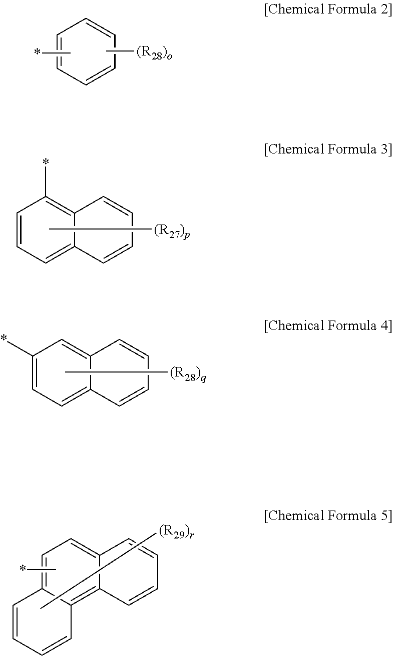
wherein in chemical formulas 2 to 5,
R₂₆ to R₂₉ are identical to or different from each other, and are each independently selected from the group consisting of hydrogen, deuterium, a substituted or unsubstituted aryl group having 6 to 30 carbon atoms, and a substituted or unsubstituted heteroaryl group having 1 to 30 carbon atoms;
o is an integer from 1 to 5, each of p and q is integer from 1 to 7, and r is an integer from 1 to 9; and
* denotes a bonding position.

7. The organic compound of claim 6, wherein when Ar₂ is selected from one of Chemical Formulas 2 to 4, Ar₁ is selected from a substituted or unsubstituted aryl group having 6 to 30 carbon atoms, and a substituted or unsubstituted aryl group having 6 to 15 carbon atoms.

8. The organic compound of claim 6, wherein when Ar₂ is selected from Chemical Formula 5, Ar₁ is selected from a substituted or unsubstituted aryl group having 6 to 30 carbon atoms, and a substituted or unsubstituted heteroaryl group having 1 to 30 carbon atoms.

9. An organic light emitting diode comprising:
an anode;
a cathode facing the anode; and
one or more organic layers disposed between the anode and the cathode,
wherein at least one of the organic layers includes the organic compound represented by chemical formula 1 of claim 1, and
wherein the organic layer including the organic compound represented by Chemical Formula 1 is a hole transport layer or a hole transport auxiliary layer.
